(19) United States
(12) Patent Application Publication
(10) Pub. No.: US 2023/0242908 A1
(43) Pub. Date: Aug. 3, 2023

(54) LNP COMPOSITIONS COMPRISING MRNA THERAPEUTICS WITH EXTENDED HALF-LIFE

(71) Applicant: ModernaTX, Inc., Cambridge, MA (US)

(72) Inventors: David Reid, Medford, MA (US); Ruchi Jain, Chestnut Hill, MA (US); Alicia Bicknell, Somerville, MA (US); Caroline Kohrer, Cambridge, MA (US)

(21) Appl. No.: 18/012,094

(22) PCT Filed: Jun. 23, 2021
(86) PCT No.: PCT/US2021/038780

§ 371 Date: Dec. 21, 2022

Related U.S. Application Data
(60) Provisional application No. 63/042,822, filed on Jun. 23, 2020.
(60) Provisional application No. 63/165,094, filed on Mar. 23, 2021.
(60) Provisional application No. 63/165,469, filed on Mar. 24, 2021.

(51) Int. Cl. C12N 15/11; A61K 9/16; C12N 15/88; C12N 15/67

(52) U.S. Cl. CPC C12N 15/11; A61K 9/1617; C12N 15/88; C12N 15/67

(57) ABSTRACT

The disclosure features a polynucleotide encoding a polypeptide, which polynucleotide comprises: a 5′UTR described herein; a coding region comprising a payload and a stop element described herein; and a 3′UTR described herein, and LNP compositions comprising the same. The polynucleotides and/or LNP compositions of the present disclosure can: increase the level and/or activity of the payload by increasing the half-life and/or duration of expression of the polynucleotide encoding the payload or of the payload polypeptide. Also disclosed herein are methods of treating a disease or disorder in a subject using the LNP compositions of the present disclosure.

### CROSS-REFERENCE TO RELATED APPLICATIONS

This application claims the benefit of U.S. Provisional Application No. 63/042,822, filed Jun. 23, 2020, U.S. Provisional Application No. 63/165,094, filed Mar. 23, 2021, and U.S. Provisional Application No. 63/165,469, filed Mar. 24, 2021. The contents of the aforesaid applications are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Jun. 23, 2021, is named M2180-7007WO_SL.txt and is 45,216 bytes in size.

### BACKGROUND OF THE DISCLOSURE

Efforts to increase mRNA potency have focused on generating mRNAs with optimal sequence design for the open reading frame (ORFs). However, there is no indication that maximum potency has been achieved with these efforts, with respect to potency and durability of mRNA expression. This is particularly true in the case of mRNA therapeutics. Therefore, there is a need to further improve potency and durability of mRNA expression by exploiting RNA biology.

### SUMMARY OF THE DISCLOSURE

The present disclosure provides, inter alia, polynucleotides encoding a polypeptide, wherein the polynucleotide comprises: (a) a 5′-UTR (e.g., as described herein); (b) a coding region comprising a stop element (e.g., as described herein); and (c) a 3′-UTR (e.g., as described herein), and LNP compositions comprising the same. In an embodiment, the coding region comprises a polynucleotide sequence, e.g., mRNA, e.g., an open reading frame (ORF) which encodes for a peptide or polypeptide payload, e.g., a therapeutic payload or a prophylactic payload. In an embodiment, the polynucleotide, e.g., mRNA, or polypeptide encoded by the polynucleotide has an increased level and/or activity, e.g., expression or half-life than versions lacking the 5′-UTRs, 3′-UTRs, or stop elements described herein. In an embodiment, the level and/or activity of the polynucleotide, e.g., mRNA, is increased. In an embodiment, the level, activity and/or duration of expression of the polypeptide encoded by the polynucleotide is increased. Also disclosed herein are methods of using an LNP composition comprising a polynucleotide disclosed herein, for treating a disease or disorder, or for promoting a desired biological effect in a subject. It will be understood that any ORF can be combined with the disclosed elements, e.g., ORFs encoding polypeptides or peptides whether, e.g., intracellular, transmembrane, or secreted. Additional aspects of the disclosure are described in further detail below.

5′ UTR and Combinations Comprising 3′ UTR and/or Stop Element

In an aspect, provided herein is a polynucleotide encoding a polypeptide (e.g., mRNA), wherein the polynucleotide comprises: (a) a 5′-UTR comprising the sequence of SEQ ID NO: 1 or a variant or fragment thereof, (b) a coding region comprising a stop element (e.g., as described herein); and (c) a 3′-UTR (e.g., as described herein).

In an embodiment, the 5′ UTR comprises a nucleic acid sequence of Formula A:

(SEQ ID NO: 46)
G G A A A U C G C A A A A (N₂)ₓ(N₃)ₓ
C U (N₄)ₓ(N₅)ₓ C G C G U U A G A U U
U C U U A G U U U U C U N₆ N₇ C A A C
U A G C A A G C U U U U U G U U C U
C G C C (N₈ C C)ₓ,

wherein:

(N₂)ₓ is a uracil and x is an integer from 0 to 5, e.g., wherein x=3 or 4; (N₃)ₓ is a guanine and x is an integer from 0 to 1; (N₄)ₓ is a cytosine and x is an integer from 0 to 1; (N₅)ₓ is a uracil and x is an integer from 0 to 5, e.g., wherein x=2 or 3; N₆ is a uracil or cytosine; N₇ is a uracil or guanine; and/or N₈ is a adenine or guanine and x is an integer from 0 to 1.

In an embodiment, the variant of SEQ ID NO: 1 comprises a sequence with at least 50%, 60%, 70%, 80%, 85%, 90%, 95%, 96%, 97%, 98%, 99% or 100% identity to SEQ ID NO: 1 or nucleotides 2-75, 3-75, 4-75, 5-75, 6-75, or 7-75 of SEQ ID NO: 1. In an embodiment, the variant of SEQ ID NO: 1 comprises a sequence with at least 50% identity to SEQ ID NO: 1 or nucleotides 2-75, 3-75, 4-75, 5-75, 6-75, or 7-75 of SEQ ID NO: 1. In an embodiment, the variant of SEQ ID NO: 1 comprises a sequence with at least 60% identity to SEQ ID NO: 1 or nucleotides 2-75, 3-75, 4-75, 5-75, 6-75, or 7-75 of SEQ ID NO: 1. In an embodiment, the variant of SEQ ID NO: 1 comprises a sequence with at least 70% identity to SEQ ID NO: 1 or nucleotides 2-75, 3-75, 4-75, 5-75, 6-75, or 7-75 of SEQ ID NO: 1. In an embodiment, the variant of SEQ ID NO: 1 comprises a sequence with at least 80% identity to SEQ ID NO: 1 or nucleotides 2-75, 3-75, 4-75, 5-75, 6-75, or 7-75 of SEQ ID NO: 1. In an embodiment, the variant of SEQ ID NO: 1 comprises a sequence with at least 90% identity to SEQ ID NO: 1 or nucleotides 2-75, 3-75, 4-75, 5-75, 6-75, or 7-75 of SEQ ID NO: 1. In an embodiment, the variant of SEQ ID NO: 1 comprises a sequence with at least 95% identity to SEQ ID NO: 1 or nucleotides 2-75, 3-75, 4-75, 5-75, 6-75, or 7-75 of SEQ ID NO: 1. In an embodiment, the variant of SEQ ID NO: 1 comprises a sequence with at least 96% identity to SEQ ID NO: 1 or nucleotides 2-75, 3-75, 4-75, 5-75, 6-75, or 7-75 of SEQ ID NO: 1. In an embodiment, the variant of SEQ ID NO: 1 comprises a sequence with at least 97% identity to SEQ ID NO: 1 or nucleotides 2-75, 3-75, 4-75, 5-75, 6-75, or 7-75 of SEQ ID NO: 1. In an embodiment, the variant of SEQ ID NO: 1 comprises a sequence with at least 98% identity to SEQ ID NO: 1 or nucleotides 2-75, 3-75, 4-75, 5-75, 6-75, or 7-75 of SEQ ID NO: 1. In an embodiment, the variant of SEQ ID NO: 1 comprises a sequence with at least 99% identity to SEQ ID NO: 1 or nucleotides 2-75, 3-75, 4-75, 5-75, 6-75, or 7-75 of SEQ ID NO: 1. In an embodiment, the variant of SEQ ID NO: 1 comprises a sequence with at least 100% identity to SEQ ID NO: 1 or nucleotides 2-75, 3-75, 4-75, 5-75, 6-75, or 7-75 of SEQ ID NO: 1.

In an embodiment, the variant of SEQ ID NO: 1 comprises a uridine content of at least 30%, 40%, 50%, 60%, 70%, or 80%. In an embodiment, the variant of SEQ ID NO: 1 comprises a uridine content of at least 30%. In an embodiment, the variant of SEQ ID NO: 1 comprises a uridine content of at least 40%. In an embodiment, the variant of SEQ ID NO: 1 comprises a uridine content of at least 50%. In an embodiment, the variant of SEQ ID NO: 1 comprises a uridine content of at least 60%. In an embodiment, the variant of SEQ ID NO: 1 comprises a uridine content of at least 70%. In an embodiment, the variant of SEQ ID NO: 1 comprises a uridine content of at least 80%.

In an embodiment, the variant of SEQ ID NO: 1 comprises at least 2, 3, 4, 5, 6, 7, 8, 9, 10, 11 or 12 consecutive uridines (e.g., a polyuridine tract). In an embodiment, the variant of SEQ ID NO: 1 comprises at least 2 consecutive uridines. In an embodiment, the variant of SEQ ID NO: 1 comprises at least 3 consecutive uridines. In an embodiment, the variant of SEQ ID NO: 1 comprises at least 4 consecutive uridines. In an embodiment, the variant of SEQ ID NO: 1 comprises at least 5 consecutive uridines. In an embodiment, the variant of SEQ ID NO: 1 comprises at least 6 consecutive uridines. In an embodiment, the variant of SEQ ID NO: 1 comprises at least 7 consecutive uridines. In an embodiment, the variant of SEQ ID NO: 1 comprises at least 8 consecutive uridines. In an embodiment, the variant of SEQ ID NO: 1 comprises at least 9 consecutive uridines. In an embodiment, the variant of SEQ ID NO: 1 comprises at least 10 consecutive uridines. In an embodiment, the variant of SEQ ID NO: 1 comprises at least 11 consecutive uridines. In an embodiment, the variant of SEQ ID NO: 1 comprises at least 12 consecutive uridines.

In an embodiment, the polyuridine tract in the variant of SEQ ID NO: 1 comprises at least at least 2-12, 2-11, 2-10, 2-9, 2-8, 2-7, 2-6, 2-5, 2-4, 2-3, 3-12, 4-12, 5-12, 6-12, 7-12, 8-12, 9-12, 10-12, 11-12, 2-6, or 3-5 consecutive uridines. In an embodiment, the polyuridine tract in the variant of SEQ ID NO: 1 comprises at least at least 2-6 consecutive uridines. In an embodiment, the polyuridine tract in the variant of SEQ ID NO: 1 comprises at least at least 2-5 consecutive uridines. In an embodiment, the polyuridine tract in the variant of SEQ ID NO: 1 comprises at least at least 2-4 consecutive uridines. In an embodiment, the polyuridine tract in the variant of SEQ ID NO: 1 comprises at least at least 3-4 consecutive uridines. In an embodiment, the polyuridine tract in the variant of SEQ ID NO: 1 comprises at least at least 3-5 consecutive uridines. In an embodiment, the polyuridine tract in the variant of SEQ ID NO: 1 comprises at least at least 4-5 consecutive uridines.

In an embodiment, the variant of SEQ ID NO: 1 comprises 2 consecutive uridines. In an embodiment, the variant of SEQ ID NO: 1 comprises 3 consecutive uridines. In an embodiment, the variant of SEQ ID NO: 1 comprises 4 consecutive uridines. In an embodiment, the variant of SEQ ID NO: 1 comprises 5 consecutive uridines. In an embodiment, the variant of SEQ ID NO: 1 comprises 6 consecutive uridines. In an embodiment, the variant of SEQ ID NO: 1 comprises 7 consecutive uridines. In an embodiment, the variant of SEQ ID NO: 1 comprises 8 consecutive uridines. In an embodiment, the variant of SEQ ID NO: 1 comprises 9 consecutive uridines. In an embodiment, the variant of SEQ ID NO: 1 comprises 10 consecutive uridines. In an embodiment, the variant of SEQ ID NO: 1 comprises 11 consecutive uridines. In an embodiment, the variant of SEQ ID NO: 1 comprises 12 consecutive uridines.

In an embodiment, the variant of SEQ ID NO: 1 comprises 1, 2, 3, 4, 5, 6, 7, 8, 9, 10, 11, 12, 13, 14, or 15 polyuridine tracts. In an embodiment, the variant of SEQ ID NO: 1 comprises 1 polyuridine tract. In an embodiment, the variant of SEQ ID NO: 1 comprises 2 polyuridine tracts. In an embodiment, the variant of SEQ ID NO: 1 comprises 3 polyuridine tracts. In an embodiment, the variant of SEQ ID NO: 1 comprises 4 polyuridine tracts. In an embodiment, the variant of SEQ ID NO: 1 comprises 5 polyuridine tracts. In an embodiment, the variant of SEQ ID NO: 1 comprises 6 polyuridine tracts. In an embodiment, the variant of SEQ ID NO: 1 comprises 7 polyuridine tracts. In an embodiment, the variant of SEQ ID NO: 1 comprises 8 polyuridine tracts.

In an embodiment, the one or more of the polyuridine tracts are adjacent to a different polyuridine tract. In an embodiment, each of, e.g., all, the polyuridine tracts are adjacent to each other, e.g., all of the polyuridine tracts are contiguous. In an embodiment, one or more of the polyuridine tracts are separated by 1, 2, 3, 4, 5, 6, 7, 8, 9, 10, 11, 2, 13, 14, 15, 16, 17, 18. 19, 20, 30, 40, 50 or 60 nucleotides. In an embodiment, each of, e.g., all of, the polyuridine tracts are separated by 1, 2, 3, 4, 5, 6, 7, 8, 9, 10, 11, 2, 13, 14, 15, 16, 17, 18. 19, 20, 30, 40, 50 or 60 nucleotides. In an embodiment, a first polyuridine tract and a second polyuridine tract are adjacent to each other.

In an embodiment, the 5′ UTR comprises a Kozak sequence, e.g., a GCCRCC nucleotide sequence (SEQ ID NO: 43) wherein R is an adenine or guanine.

In an embodiment, the 5′ UTR comprises the sequence of SEQ ID NO: 1 or a sequence with at least 80%, 85%, 90%, 95%, 96%, 97%, 98%, 99% or 100% identity to SEQ ID NO: 1 or nucleotides 2-75, 3-75, 4-75, 5-75, 6-75, or 7-75 of SEQ ID NO: 1. In an embodiment, the 5′ UTR comprises the sequence of SEQ ID NO: 1 or nucleotides 2-75, 3-75, 4-75, 5-75, 6-75, or 7-75 of SEQ ID NO: 1.

In an embodiment, the 5′ UTR comprises the sequence of SEQ ID NO: 41 or a sequence with at least 80%, 85%, 90%, 95%, 96%, 97%, 98%, 99% or 100% identity to SEQ ID NO: 41 or nucleotides 2-81, 3-81, 4-81, 5-81, 6-81, or 7-81 of SEQ ID NO: 41. In an embodiment, the 5′ UTR comprises the sequence of SEQ ID NO: 41 or nucleotides 2-81, 3-81, 4-81, 5-81, 6-81, or 7-81 of SEQ ID NO: 41.

In an embodiment, the 5′ UTR comprises the sequence of SEQ ID NO: 42 or a sequence with at least 80%, 85%, 90%, 95%, 96%, 97%, 98%, 99% or 100% identity to SEQ ID NO: 42 or nucleotides 2-81, 3-81, 4-81, 5-81, 6-81, or 7-81 of SEQ ID NO: 42. In an embodiment, the 5′ UTR comprises the sequence of SEQ ID NO: 42 or nucleotides 2-81, 3-81, 4-81, 5-81, 6-81, or 7-81 of SEQ ID NO: 42.

In an embodiment, the 5′ UTR results in an increased half-life of the polynucleotide, e.g., about 1.5-20-fold increase in half-life of the polynucleotide. In an embodiment, the increase in half-life of the polynucleotide is compared to an otherwise similar polynucleotide which does not have a 5′ UTR, has a different 5′ UTR, or does not have a 5′ UTR disclosed herein. In an embodiment, the increase in half-life of the polynucleotide is measured according to an assay that measures the half-life of a polynucleotide, e.g., an assay described in any one of the Examples herein.

In an embodiment, the 5′ UTR results in an increased level and/or activity, e.g., output, of the polypeptide encoded by the polynucleotide. In an embodiment, the 5′ UTR results in about 1.5-20-fold increase in level and/or activity, e.g., output, of the polypeptide encoded by the polynucleotide. In an embodiment, the increase in activity is compared to an otherwise similar polynucleotide which does not have a 5′ UTR, has a different 5′ UTR, or does not have the 5′ UTR disclosed herein.

In an embodiment, the coding region of (b) comprises a stop element chosen from a stop element provided in Table 3, e.g., SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO:36, SEQ ID NO: 37, SEQ ID NO: 56, SEQ ID NO: 57, SEQ ID NO: 62, SEQ ID NO: 93 or SEQ ID NO: 96.

In an embodiment, the 3′ UTR of (c) comprises a 3′ UTR sequence provided in Table 2 or a sequence with at least 80%, 85%, 90%, 95%, 96%, 97%, 98%, 99% or 100% identity to a 3′ UTR sequence provided in Table 2, or a fragment thereof (e.g., a fragment that lacks the first (i.e., 5′ most) one, two, three, four, five, six, or more nucleotides of the 3′ UTR sequence provided in Table 2). In an embodiment, the 3′ UTR comprises a sequence with at least 80%, 85%, 90%, 95%, 96%, 97%, 98%, 99% or 100% identity to SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO:45, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 94 or SEQ ID NO: 95, or a fragment thereof (e.g., a fragment that lacks the first one, two, three, four, five, six, or more nucleotides of any of the aforesaid sequences). In an embodiment, the 3′ UTR comprises a micro RNA (miRNA) binding site, e.g., as described herein, e.g., a sequence of any one of SEQ ID NOs: 38-40.

In an embodiment, the 3′ UTR comprises one or more (e.g., 2 or 3) of a TENT recruiting sequence described herein. In an embodiment, the 3′ UTR comprises a sequence with at least 80%, 85%, 90%, 95%, 96%, 97%, 98%, 99% or 100% identity to SEQ ID NO: 91 or 92.

In an embodiment, (i) the coding region of (b) comprises a stop element chosen from a stop element provided in Table 3, e.g., SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO:36, SEQ ID NO: 37, SEQ ID NO: 56, SEQ ID NO: 57, SEQ ID NO: 62, SEQ ID NO: 93 or SEQ ID NO: 96; and (ii) the 3′ UTR of (c) comprises a 3′ UTR sequence provided in Table 2 or a sequence with at least 80%, 85%, 90%, 95%, 96%, 97%, 98%, 99% or 100% identity to a 3′ UTR sequence provided in Table 2, or a fragment thereof (e.g., a fragment that lacks the first one, two, three, four, five, six, or more nucleotides of the 3′ UTR sequence provided in Table 2).

In an embodiment, the 3′ UTR comprises a sequence with at least 80%, 85%, 90%, 95%, 96%, 97%, 98%, 99% or 100% identity to SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO:45, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 94 or SEQ ID NO: 95, or a fragment thereof (e.g., a fragment that lacks the first one, two, three, four, five, six, or more nucleotides of any of the aforesaid sequences). In an embodiment, the 3′ UTR comprises a micro RNA binding site, e.g., as described herein, e.g., a sequence of any one of SEQ ID NOs: 38-40.

In an embodiment, the 3′ UTR comprises one or more (e.g., 2 or 3) of a TENT recruiting sequence described herein. In an embodiment, the 3′ UTR comprises a sequence with at least 80%, 85%, 90%, 95%, 96%, 97%, 98%, 99% or 100% identity to SEQ ID NO: 91 or 92.

3′ UTR and Combinations Comprising 5′ UTR and/or Stop Element

In an aspect, disclosed herein is a polynucleotide encoding a polypeptide, wherein the polynucleotide comprises: (a) a 5′-UTR (e.g., as described herein); (b) a coding region comprising a stop element (e.g., as described herein); and (c) a 3′ UTR comprising a core sequence with at least 80%, 85%, 90%, 95%, 96%, 97%, 98%, 99% or 100% identity to SEQ ID NO: 11 or a fragment thereof.

In an embodiment, the 3′ UTR core sequence is disposed immediately downstream of the stop element of (b). In an embodiment, the 3′ UTR core sequence is disposed at the C terminus end of the polynucleotide.

In an embodiment, the 3′ UTR comprising a core sequence comprises a first flanking sequence. In an embodiment, the 3′ UTR comprising a core sequence comprises a second flanking sequence. In an embodiment, the 3′ UTR comprising a core sequence comprises a first flanking sequence and a second flanking sequence.

In an embodiment, the first flanking sequence comprises a sequence of about 5-25, about 5-20, about 5-15, about 5-10, about 10-25, about 15-25, about 20-25 nucleotides. In an embodiment, the first flanking sequence comprises a sequence of about 5, 6, 7, 8, 9, 10, 11, 12, 13, 14, 15, 16, 17, 18, 19, 20, 21, 22, 23, 24, or 25 nucleotides, e.g., 11 nucleotides.

In an embodiment, the second flanking sequence comprises a sequence of about 20-80, about 20-75, about 20-70, about 20-65, about 20-60, about 20-55, about 20-50, about 20-45, bout 20-40, about 20-35, about 20-30, about 20-25, about 25-80, about 30-80, about 35-80, about 40-80, about 45-80, about 50-80, about 55-80, about 60-80, about 65-80, about 70-80 or about 75-80 nucleotides. In an embodiment, the second flanking sequence comprises a sequence of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, or 80 nucleotides, e.g., 39 nucleotides.

In an embodiment, the first flanking sequence is upstream or downstream of the core sequence. In an embodiment, the second flanking sequence is upstream or downstream of the core sequence.

In an embodiment, the 3′ UTR comprises a fragment of SEQ ID NO: 11, e.g., a 5 nucleotide (nt), 10 nt, 15 nt, 20 nt, 25 nt, 30 nt, 35 nt, 40 nt, 45 nt, 50 nt, 55 nt, 60 nt, 65 nt, or 70 nt fragment of SEQ ID NO: 11. In an embodiment, the 3′ UTR comprises 15-25 nt fragment comprising a 60 nt fragment of SEQ ID NO: 11.

In an embodiment, the 3′ UTR comprises the sequence of SEQ ID NO: 45 or a fragment thereof. In an embodiment, the 3′ UTR comprises the sequence of SEQ ID NO: 11 or a fragment thereof.

In an embodiment, the 3′ UTR results in an increased half-life of the polynucleotide, e.g., about 1.5-10-fold increase in half-life of the polynucleotide, e.g., as measured by an assay that measures the half-life of a polynucleotide, e.g., an assay of any one of Examples disclosed herein.

In an embodiment, the 3′ UTR results in a polynucleotide with a mean half-life score of greater than 10. In an embodiment, the 3′ UTR results in an increased level and/or activity, e.g., output, of the polypeptide encoded by the polynucleotide. In an embodiment, the increase is compared to an otherwise similar polynucleotide which does not have a 3′ UTR, has a different 3′ UTR, or does not have the 3′ UTR disclosed herein.

In an embodiment, the 3′ UTR comprises a micro RNA (miRNA) binding site, e.g., as described herein. In an embodiment, the 3′ UTR comprises a miRNA binding site of SEQ ID NO: 38, SEQ ID NO: 39, SEQ ID NO: 40 or a combination thereof. In an embodiment, the 3′ UTR comprises a plurality of miRNA binding sites, e.g., 2, 3, 4, 5, 6, 7 or 8 miRNA binding sites. In an embodiment, the plurality of miRNA binding sites comprises the same or different miRNA binding sites.

In an embodiment, the 5′ UTR of (a) comprises a 5′ UTR sequence provided in Table 1 or a sequence with at least 80%, 85%, 90%, 95%, 96%, 97%, 98%, 99% or 100% identity to a 5′ UTR sequence provided in Table 1, or a fragment thereof (e.g., a fragment that lacks the first (i.e., 5′ most) one, two, three, four, five, or six nucleotides of the 5′ UTR sequence provided in Table 1). In an embodiment, the 5′ UTR comprises a sequence with at least 80%, 85%, 90%, 95%, 96%, 97%, 98%, 99% or 100% identity to SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 41, SEQ ID NO: 42, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 88, SEQ ID NO: 89 or SEQ ID NO: 90 or a fragment thereof (e.g., a fragment that lacks the first one, two, three, four, five, or six nucleotides of any of the aforesaid sequences).

In an embodiment, the coding region of (b) comprises a stop element sequence provided in Table 3. In an embodiment, the coding region of (b) comprises a stop element chosen from a stop element provided in Table 3, e.g., SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO:36, SEQ ID NO: 37, SEQ ID NO: 56, SEQ ID NO: 57, SEQ ID NO: 62, SEQ ID NO: 93 or SEQ ID NO: 96.

In an embodiment, (i) the 5′ UTR of (a) comprises a 5′ UTR sequence provided in Table 1 or a sequence with at least 80%, 85%, 90%, 95%, 96%, 97%, 98%, 99% or 100% identity to a 5′ UTR sequence provided in Table 1, or a fragment thereof (e.g., a fragment that lacks the first one, two, three, four, five, or six nucleotides of the 5′ UTR sequence provided in Table 1); and (ii) the stop element of (b) comprises a stop element sequence provided in Table 3.

In an embodiment, the 5′ UTR comprises a sequence with at least 80%, 85%, 90%, 95%, 96%, 97%, 98%, 99% or 100% identity to SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 41, SEQ ID NO: 42, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 88, SEQ ID NO: 89 or SEQ ID NO: 90 or a fragment thereof (e.g., a fragment that lacks the first one, two, three, four, five, or six nucleotides of any of the aforesaid sequences).

In an embodiment, the coding region of (b) comprises a stop element chosen from a stop element provided in Table 3, e.g., SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO:36, SEQ ID NO: 56, SEQ ID NO: 57, SEQ ID NO: 62, SEQ ID NO: 93 or SEQ ID NO:96.

Stop Element and Combinations Comprising 5′ UTR and/or Stop Elements

In another aspect, provided herein is a polynucleotide encoding a polypeptide, wherein the polynucleotide comprises: (a) a 5′-UTR (e.g., as described herein); (b) a coding region comprising a stop element chosen from a stop element provided in Table 3; and (c) a 3′-UTR (e.g., as described herein).

In an embodiment, the stop element comprises the sequence of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO:36, SEQ ID NO: 93 or SEQ ID NO: 96.

In an embodiment, the coding region of (b) comprises a stop element comprising a consensus sequence of Formula B:

(SEQ ID NO: 37)
X₋₃-X₋₂-X₋₁-U-A-A-X₁-X₂-X₃-X₄-X₅-X₆-X₇-X₈-X₉-X₁₀-X₁₁-X₁₂

wherein:

X₁ is a G or A;

X₂, X₄, X₅ X₆ or X₇ is each independently C or U;

X₃ is C or A;

X₈, X₁₀, X₁₁, X₁₂ X₋₁ or X₋₃ is each independently C or G;

X₉ is G or U; and/or

X₋₂ is A or U.

In an embodiment, the coding region of (b) comprises a stop element comprising a consensus sequence of Formula C:

(SEQ ID NO: 56)
X₋₃-X₋₂-X₋₁-U-G-A-X₁-X₂-X₃-X₄-X₅-X₆-X₇-X₈-X₉-X₁₀-X₁₁-X₁₂

wherein:

X₋₃, X₋₁, X₂, X₅, X₆, X₇, X₈, X₉, or X₁₂ is each independently G or C;

X₋₂, X₃, or X₄ is each independent A or C;

X₁ is A or G; and/or

X₁₀ or X₁₁ is each independently C or U.

In an embodiment, the coding region of (b) comprises a stop element comprising a consensus sequence of Formula D

(SEQ ID NO: 57)
X₋₃-X₋₂-X₋₁-U-A-G-X₁-X₂-X₃-X₄-X₅-X₆-X₇-X₈-X₉-X₁₀-X₁₁-X₁₂

wherein:

X₋₃, X₋₁, X₂, X₃, X₁₀ is each independently G or C;

X₋₂ or X₉ is each independently A or U;

X₁ or X₄ is each independently A or G;

X₅ or X₈ is each independently A or C; and/or

X₆, X₇, X₁₁ or X₁₂ is each independently C or U.

In an embodiment, the consensus sequence has a high GC content, e.g., GC content of about 50%, 60%, 70%, 80%, 90% or 99%.

In an embodiment, the stop element results in an increased half-life of the polynucleotide, e.g., about 1.5-20-fold increase in half-life of the polynucleotide. In an embodiment, the increase in half-life of the polynucleotide is compared to an otherwise similar polynucleotide which does not have a stop element, has a different stop element, or does not have a stop element disclosed herein. In an embodiment, the increase in half-life of the polynucleotide is measured according to an assay which measures the half-life of a polynucleotide, e.g., an assay described in any one of Examples disclosed herein.

In an embodiment, the stop element results in an increased level and/or activity, e.g., output or duration of expression, of the polypeptide encoded by the polynucleotide. In an embodiment, the increase in level and/or activity, e.g., output or duration of expression, of the polypeptide is measured according to an assay which measures the level and/or activity, e.g., output or duration of expression of a polypeptide, e.g., an assay described in any one of Examples disclosed herein. In an embodiment, the stop element results in about 1.5-20-fold increase in level and/or activity, e.g., output, of the polypeptide encoded by the polynucleotide. In an embodiment, the stop element results in about 1.5-20-fold increase in level and/or activity, e.g., detectable level or activity, of the polypeptide encoded by the polynucleotide for about 1, 2, 3, 4, 5, 6, 7, 8, 9, 10, or 14 days. In an embodiment, the stop element results in detectable level or activity of the polypeptide encoded by the polynucleotide for about 1, 2, 3, 4, 5, 6, 7, 8, 9, 10, or 14 days.

In an embodiment, the increase is compared to an otherwise similar polynucleotide which does not have a stop element, has a different stop element, or does not have a stop element disclosed herein.

In an embodiment, the 5′ UTR of (a) comprises a 5′ UTR sequence provided in Table 1 or a sequence with at least 80%, 85%, 90%, 95%, 96%, 97%, 98%, 99% or 100% identity to a 5′ UTR sequence provided in Table 1, or a variant or a fragment thereof (e.g., a fragment that lacks the first one, two, three, four, five, or six nucleotides of the 5′ UTR sequence provided in Table 1). In an embodiment, the 5′ UTR comprises a sequence with at least 80%, 85%, 90%, 95%, 96%, 97%, 98%, 99% or 100% identity to SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 41, SEQ ID NO: 42, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 88, SEQ ID NO: 89 or SEQ ID NO: 90, or a fragment thereof (e.g., a fragment that lacks the first one, two, three, four, five, or six nucleotides of any of the aforesaid sequences).

In an embodiment, the 3′ UTR of (c) comprises a 3′ UTR sequence provided in Table 2 or a sequence with at least 80%, 85%, 90%, 95%, 96%, 97%, 98%, 99% or 100% identity to a 3′ UTR sequence provided in Table 2, or a fragment thereof (e.g., a fragment that lacks the first one, two, three, four, five, six, or more nucleotides of the 3′ UTR sequence provided in Table 2). In an embodiment, the 3′ UTR comprises a sequence with at least 80%, 85%, 90%, 95%, 96%, 97%, 98%, 99% or 100% identity to SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 45, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 94 or SEQ ID NO: 95, or a fragment thereof (e.g., a fragment that lacks the first one, two, three, four, five, six, or more nucleotides of any of the aforesaid sequences). In an embodiment, the 3′ UTR comprises a micro RNA binding site, e.g., as described herein, e.g., a sequence of any one of SEQ ID NOs: 38-40.

In an embodiment, the 3′ UTR comprises one or more (e.g., 2 or 3) of a TENT recruiting sequence described herein. In an embodiment, the 3′ UTR comprises a sequence with at least 80%, 85%, 90%, 95%, 96%, 97%, 98%, 99% or 100% identity to SEQ ID NO: 91 or 92.

In an embodiment, (i) the 5′ UTR of (a) comprises a 5′ UTR sequence provided in Table 1 or a sequence with at least 80%, 85%, 90%, 95%, 96%, 97%, 98%, 99% or 100% identity to a 3′ UTR sequence provided in Table 1, or a fragment thereof (e.g., a fragment that lacks the first one, two, three, four, five, or six nucleotides of the 5′ UTR sequence provided in Table 1); and (ii) the 3′ UTR of (c) comprises a 3′ UTR sequence provided in Table 2 or a sequence with at least 80%, 85%, 90%, 95%, 96%, 97%, 98%, 99% or 100% identity to a 3′ UTR sequence provided in Table 2, or a fragment thereof (e.g., a fragment that lacks the first one, two, three, four, five, six, or more nucleotides of the 3′ UTR sequence provided in Table 2).

In an embodiment, the 5′ UTR comprises a sequence with at least 80%, 85%, 90%, 95%, 96%, 97%, 98%, 99% or 100% identity to SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 41, SEQ ID NO: 42, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 88, SEQ ID NO: 89 or SEQ ID NO: 90, or a fragment thereof (e.g., a fragment that lacks the first one, two, three, four, five, or six nucleotides of any of the aforesaid sequences).

In an embodiment, the 3′ UTR comprises a sequence with at least 80%, 85%, 90%, 95%, 96%, 97%, 98%, 99% or 100% identity to SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO:45, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 94 or SEQ ID NO: 95, or a fragment thereof (e.g., a fragment that lacks the first one, two, three, four, five, six, or more nucleotides of any of the aforesaid sequences). In an embodiment, the 3′ UTR comprises a micro RNA binding site, e.g., as described herein, e.g., a sequence of any one of SEQ ID NOs: 38-40.

In an embodiment, the 3′ UTR comprises one or more (e.g., 2 or 3) of a TENT recruiting sequence described herein. In an embodiment, the 3′ UTR comprises a sequence with at least 80%, 85%, 90%, 95%, 96%, 97%, 98%, 99% or 100% identity to SEQ ID NO: 91 or 92.

Optional Features of mRNA Constructs Disclosed Herein

In an embodiment, the coding region of the polynucleotide comprises a sequence encoding a therapeutic payload or a prophylactic payload.

In an embodiment, the therapeutic payload or prophylactic payload comprises a secreted protein, a membrane-bound protein; or an intercellular protein.

In an embodiment, the therapeutic payload or prophylactic payload is chosen from a cytokine, an antibody, a vaccine (e.g., an antigen, an immunogenic epitope), a receptor, an enzyme, a hormone, a transcription factor, a ligand, a membrane transporter, a structural protein, a nuclease, or a component, variant or fragment (e.g., a biologically active fragment) thereof. In an embodiment, the therapeutic payload or prophylactic payload comprises a protein or peptide.

In an embodiment, the polynucleotide further comprises at least one 5′ cap structure, e.g., as described herein, and/or a poly A tail, e.g., as described herein.

In an embodiment, the 5′ cap structure comprises a sequence of GG, GA, or GGA, wherein the underlined, italicized G is an in inverted G nucleotide followed by a 5′-5′-triphosphate group.

In an embodiment, the polynucleotide further comprises a 3′ stabilizing region, e.g., a stabilized tail e.g., as described herein. In an embodiment, the 3′ stabilizing region comprises a poly A tail, e.g., a poly A tail comprising 80-150, e.g., 120, adenines (SEQ ID NO: 123). In an embodiment, the poly A tail comprises one or more non-adenosine residues, e.g., one or more guanosines, e.g., as described herein. In an embodiment, the poly A tail comprises a UCUAG sequence (SEQ ID NO: 44). In an embodiment, the poly A tail comprises about 80-120, e.g., 100, adenines upstream of SEQ ID NO: 44. In an embodiment, the poly A tail comprises about 1-40, e.g., 20, adenines downstream of SEQ ID NO: 44.

In an embodiment, the 3′ stabilizing region comprises at least one alternative nucleoside, optionally wherein the alternative nucleoside is an inverted thymidine (idT).

In an embodiment, the 3′ stabilizing region comprises a structure of Formula VII:

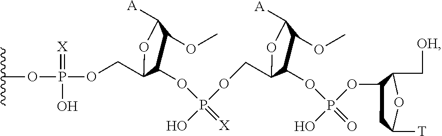

or a salt thereof, wherein each X is independently O or S, and A represents adenine and T represents thymine.

In an embodiment, the polynucleotide comprises an mRNA.

### LNP Compositions and Methods of Use

In another aspect, disclosed herein is a lipid nanoparticle (LNP) composition comprising a polynucleotide disclosed herein.

In one aspect, disclosed herein is a pharmaceutical composition comprising an LNP composition comprising a polynucleotide disclosed herein.

In an aspect, the disclosure provides a cell comprising an LNP composition comprising a polynucleotide disclosed herein.

In yet another aspect, provided herein is a method of increasing expression of a payload, e.g., a therapeutic payload or a prophylactic payload in a cell, comprising administering to the cell an LNP composition comprising a polynucleotide disclosed herein.

In a related aspect, provided herein is a composition comprising an LNP composition comprising a polynucleotide disclosed herein for use in a method of increasing expression of a payload, e.g., a therapeutic payload or a prophylactic payload in a cell.

In an aspect, provided herein is a method of delivering an LNP composition comprising a polynucleotide disclosed herein. In an embodiment, the method comprises contacting the cell in vitro, in vivo or ex vivo with the LNP composition.

In an aspect, provided herein is a method of delivering an LNP composition comprising a polynucleotide disclosed herein to a subject having a disease or disorder, e.g., as described herein.

In another aspect, provided herein is a method of modulating an immune response in a subject, comprising administering to the subject in need thereof an effective amount of an LNP composition comprising a polynucleotide disclosed herein.

In a related aspect, the disclosure provides a composition comprising an LNP composition comprising a polynucleotide disclosed herein for use in a method of modulating an immune response in a subject.

In an aspect, provided herein is a method of treating, preventing, or preventing a symptom of, a disease or disorder comprising administering to a subject in need thereof an effective amount of an LNP composition comprising a polynucleotide disclosed herein.

In a related aspect, the disclosure provides a composition comprising an LNP composition comprising a polynucleotide disclosed herein for use in a method of treating, preventing, or preventing a symptom of, a disease or disorder.

In an embodiment of any of the methods or compositions for use disclosed herein, the LNP is formulated for intravenous, subcutaneous, intramuscular, intranasal, intraocular, rectal, pulmonary or oral delivery.

In an embodiment of any of the methods or compositions for use disclosed herein, the subject is a mammal, e.g., a human.

In an embodiment of any of the methods or compositions for use disclosed herein, the subject has a disease or disorder disclosed herein.

### Additional Features of Compositions and Methods Disclosed Herein

Additional features of any of the LNP compositions, pharmaceutical composition comprising said LNPs, methods or compositions for use disclosed herein include the following embodiments.

In some embodiments of any of the methods or compositions disclosed herein, the LNP composition comprises: (i) an ionizable lipid, e.g., an amino lipid; (ii) a sterol or other structural lipid; (iii) a non-cationic helper lipid or phospholipid; and (iv) a PEG-lipid. In some embodiments, the ionizable lipid comprises a compound of Formula (IIa). In some embodiments, the ionizable lipid comprises a compound of Formula (IIe).

In some embodiments of any of the LNP compositions, methods or uses disclosed herein, the coding region of the polynucleotide comprises a sequence encoding: a secreted protein, a membrane-bound protein; or an intercellular protein.

In some embodiments, the therapeutic payload or prophylactic payload is chosen from a cytokine, an antibody, a vaccine (e.g., an antigen, an immunogenic epitope), a receptor, an enzyme, a hormone, a transcription factor, a ligand, a membrane transporter, a structural protein, a nuclease, or a component, variant or fragment (e.g., a biologically active fragment) thereof.

In some embodiments, the therapeutic payload or prophylactic payload comprises a cytokine, or a variant or fragment (e.g., a biologically active fragment) thereof.

In some embodiments, the therapeutic payload or prophylactic payload comprises an antibody or a variant or fragment (e.g., a biologically active fragment) thereof.

In some embodiments, the therapeutic payload or prophylactic payload comprises a vaccine (e.g., an antigen, an immunogenic epitope), or a component, variant or fragment (e.g., a biologically active fragment) thereof.

In some embodiments, the therapeutic payload or prophylactic payload comprises a protein or peptide.

In some embodiments of any of the LNP compositions, methods or uses disclosed herein, the polynucleotide comprises an mRNA. In some embodiments, the mRNA comprises at least one chemical modification, e.g., as described herein. In an embodiment, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine. In an embodiment, the chemical modification is selected from the group consisting of pseudouridine, N1-methylpseudouridine, 5-methylcytosine, 5-methoxyuridine, and a combination thereof. In an embodiment, the chemical modification is N1-methylpseudouridine. In an embodiment, each mRNA in the lipid nanoparticle comprises fully modified N1-methylpseudouridine.

In some embodiments of any of the LNP compositions, methods or uses disclosed herein, the LNP is formulated for intravenous, subcutaneous, intramuscular, intranasal, intraocular, rectal, pulmonary or oral delivery. In some embodiments, the LNP is formulated for intravenous delivery. In some embodiments, the LNP is formulated for subcutaneous delivery. In some embodiments, the LNP is formulated for intramuscular delivery. In some embodiments, the LNP is formulated for intranasal delivery. In some embodiments, the LNP is formulated for intraocular delivery. In some embodiments, the LNP is formulated for rectal delivery. In some embodiments, the LNP is formulated for pulmonary delivery. In some embodiments, the LNP is formulated for oral delivery.

In some embodiments of any of the LNP compositions, methods or uses disclosed herein, the LNP further comprising a pharmaceutically acceptable carrier or excipient.

In an embodiment of any of the LNP compositions, methods or compositions for use disclosed herein, the LNP composition comprises: (i) an ionizable lipid, e.g., an amino lipid; (ii) a sterol or other structural lipid; (iii) a non-cationic helper lipid or phospholipid; and, optionally, (iv) a PEG-lipid.

In an embodiment of any of the LNP compositions, methods or compositions for use disclosed herein, the LNP composition comprises an ionizable lipid comprising an amino lipid.

In an embodiment, the ionizable lipid comprises a compound of any of Formulae (I), (IA), (IB), (II), (IIa), (IIb), (IIc), (IId), (IIe), (IIf), (IIg), (III), (IIIa1), (IIIa2), (IIIa3), (IIIa4), (IIIa5), (IIIa6), (IIIa7), or (IIIa8). In an embodiment, the ionizable lipid comprises a compound of Formula (I). In an embodiment, the ionizable lipid comprises a compound of Formula (IIa). In an embodiment, the ionizable lipid comprises a compound of Formula (IIe).

In an embodiment of any of the LNP compositions, methods or compositions for use disclosed herein, the LNP composition comprises a non-cationic helper lipid or phospholipid comprising a compound selected from the group consisting of 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In an embodiment, the phospholipid is DSPC, e.g., a variant of DSPC, e.g., a compound of Formula (IV).

In an embodiment of any of the LNP compositions, methods or compositions for use disclosed herein, the LNP composition comprises a structural lipid. In one embodiment, the structural lipid is a phytosterol or a combination of a phytosterol and cholesterol. In one embodiment, the phytosterol is selected from the group consisting of 0-sitosterol, stigmasterol, 0-sitostanol, campesterol, brassicasterol, and combinations thereof.

In one embodiment, the structural lipid can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In one embodiment, the structural lipid is selected from selected from β-sitosterol and cholesterol. In an embodiment, the structural lipid is p-sitosterol. In an embodiment, the structural lipid is cholesterol.

In an embodiment of any of the LNP compositions, methods or compositions for use disclosed herein, the LNP composition comprises a PEG lipid. In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In an embodiment, the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In an embodiment, the PEG lipid is selected from the group consisting of PEG-c-DOMG, PEG-DMG, PEG-DLPE, PEG-DMPE, PEG-DPPC and PEG-DSPE lipid. In an embodiment, the PEG-lipid is PEG-DMG.

In an embodiment, the PEG lipid is chosen from a compound of: Formula (V), Formula (VI-A), Formula (VI-B), Formula (VI-C) or Formula (VI-D). In an embodiment, the PEG-lipid is a compound of Formula (VI-A). In an embodiment, the PEG-lipid is a compound of Formula (VI-B). In an embodiment, the PEG-lipid is a compound of Formula (VI-C). In an embodiment, the PEG-lipid is a compound of Formula (VI-D).

In an embodiment of any of the LNP compositions, methods or compositions for use disclosed herein, the LNP comprises about 20 mol % to about 60 mol % ionizable lipid, about 5 mol % to about 25 mol % non-cationic helper lipid or phospholipid, about 25 mol % to about 55 mol % sterol or other structural lipid, and about 0.5 mol % to about 15 mol % PEG lipid. In one embodiment of the LNPs or methods of the disclosure, the LNP comprises about 35 mol % to about 55 mol % ionizable lipid, about 5 mol % to about 25 mol % non-cationic helper lipid or phospholipid, about 30 mol % to about 40 mol % sterol or other structural lipid, and about 0 mol % to about 10 mol % PEG lipid. In one embodiment of the LNPs or methods of the disclosure, the LNP comprises about 50 mol % ionizable lipid, about 10 mol % non-cationic helper lipid or phospholipid, about 38.5 mol % sterol or other structural lipid, and about 1.5 mol % PEG lipid. In one embodiment of the LNPs or methods of the disclosure, the LNP comprises about 49.83 mol % ionizable lipid, about 9.83 mol % non-cationic helper lipid or phospholipid, about 30.33 mol % sterol or other structural lipid, and about 2.0 mol % PEG lipid.

In an embodiment of any of the LNP compositions, methods or compositions for use disclosed herein, the LNP comprises about 45 mol % to about 50 mol % ionizable lipid. In one embodiment of the LNPs or methods of the disclosure, the LNP comprises about 45.5 mol % to about 49.5 mol % ionizable lipid. In one embodiment of the LNPs or methods of the disclosure, the LNP comprises about 46 mol % to about 49 mol % ionizable lipid. In one embodiment of the LNPs or methods of the disclosure, the LNP comprises about 46.5 mol % to about 48.5 mol % ionizable lipid. In one embodiment of the LNPs or methods of the disclosure, the LNP comprises about 47 mol % to about 48 mol % ionizable lipid.

In an embodiment of any of the LNP compositions, methods or compositions for use disclosed herein, the LNP comprises about 45 mol % to about 49.5 mol % ionizable lipid. In one embodiment of the LNPs or methods of the disclosure, the LNP comprises about 45 mol % to about 49 mol % ionizable lipid. In one embodiment of the LNPs or methods of the disclosure, the LNP comprises about 45 mol % to about 48.5 mol % ionizable lipid. In one embodiment of the LNPs or methods of the disclosure, the LNP comprises about 45 mol % to about 48 mol % ionizable lipid. In one embodiment of the LNPs or methods of the disclosure, the LNP comprises about 45 mol % to about 47.5 mol % ionizable lipid. In one embodiment of the LNPs or methods of the disclosure, the LNP comprises about 45 mol % to about 47 mol % ionizable lipid. In one embodiment of the LNPs or methods of the disclosure, the LNP comprises about 45 mol % to about 46.5 mol % ionizable lipid. In one embodiment of the LNPs or methods of the disclosure, the LNP comprises about 45 mol % to about 46 mol % ionizable lipid. In one embodiment of the LNPs or methods of the disclosure, the LNP comprises about 45 mol % to about 45.5 mol % ionizable lipid.

In an embodiment of any of the LNP compositions, methods or compositions for use disclosed herein, the LNP comprises about 45.5 mol % to about 50 mol % ionizable lipid. In one embodiment of the LNPs or methods of the disclosure, the LNP comprises about 46 mol % to about 50 mol % ionizable lipid. In one embodiment of the LNPs or methods of the disclosure, the LNP comprises about 46.5 mol % to about 50 mol % ionizable lipid. In one embodiment of the LNPs or methods of the disclosure, the LNP comprises about 47 mol % to about 50 mol % ionizable lipid. In one embodiment of the LNPs or methods of the disclosure, the LNP comprises about 47.5 mol % to about 50 mol % ionizable lipid. In one embodiment of the LNPs or methods of the disclosure, the LNP comprises about 48 mol % to about 50 mol % ionizable lipid. In one embodiment of the LNPs or methods of the disclosure, the LNP comprises about 48.5 mol % to about 50 mol % ionizable lipid. In one embodiment of the LNPs or methods of the disclosure, the LNP comprises about 49 mol % to about 50 mol % ionizable lipid. In one embodiment of the LNPs or methods of the disclosure, the LNP comprises about 49.5 mol % to about 50 mol % ionizable lipid.

In an embodiment of any of the LNP compositions, methods or compositions for use disclosed herein, the LNP comprises about 45 mol % to about 46 mol % ionizable lipid. In one embodiment of the LNPs or methods of the disclosure, the LNP comprises about 45.5 mol % to about 46.5 mol % ionizable lipid. In one embodiment of the LNPs or methods of the disclosure, the LNP comprises about 46 mol % to about 47 mol % ionizable lipid. In one embodiment of the LNPs or methods of the disclosure, the LNP comprises about 46.5 mol % to about 47.5 mol % ionizable lipid. In one embodiment of the LNPs or methods of the disclosure, the LNP comprises about 47 mol % to about 48 mol % ionizable lipid. In one embodiment of the LNPs or methods of the disclosure, the LNP comprises about 47.5 mol % to about 48.5 mol % ionizable lipid. In one embodiment of the LNPs or methods of the disclosure, the LNP comprises about 48 mol % to about 49 mol % ionizable lipid. In one embodiment of the LNPs or methods of the disclosure, the LNP comprises about 48.5 mol % to about 49.5 mol % ionizable lipid. In one embodiment of the LNPs or methods of the disclosure, the LNP comprises about 49 mol % to about 50 mol % ionizable lipid.

In an embodiment of any of the LNP compositions, methods or compositions for use disclosed herein, the LNP comprises about 45 mol % ionizable lipid. In one embodiment of the LNPs or methods of the disclosure, the LNP comprises about 45.5 mol % ionizable lipid. In one embodiment of the LNPs or methods of the disclosure, the LNP comprises about 46 mol % ionizable lipid. In one embodiment of the LNPs or methods of the disclosure, the LNP comprises about 46.5 mol % ionizable lipid. In one embodiment of the LNPs or methods of the disclosure, the LNP comprises about 47 mol % ionizable lipid. In one embodiment of the LNPs or methods of the disclosure, the LNP comprises about 47.5 mol % ionizable lipid. In one embodiment of the LNPs or methods of the disclosure, the LNP comprises about 48 mol % ionizable lipid. In one embodiment of the LNPs or methods of the disclosure, the LNP comprises about 48.5 mol % ionizable lipid. In one embodiment of the LNPs or methods of the disclosure, the LNP comprises about 49 mol % ionizable lipid. In one embodiment of the LNPs or methods of the disclosure, the LNP comprises about 49.5 mol % ionizable lipid. In one embodiment of the LNPs or methods of the disclosure, the LNP comprises about 50 mol % ionizable lipid.

In an embodiment of any of the LNP compositions, methods or compositions for use disclosed herein, the LNP comprises about 1 mol % to about 5 mol % PEG lipid. In one embodiment of the LNPs or methods of the disclosure, the LNP comprises about 1.5 mol % to about 4.5 mol % PEG lipid. In one embodiment of the LNPs or methods of the disclosure, the LNP comprises about 2 mol % to about 4 mol % PEG lipid. In one embodiment of the LNPs or methods of the disclosure, the LNP comprises about 2.5 mol % to about 3.5 mol % PEG lipid.

In an embodiment of any of the LNP compositions, methods or compositions for use disclosed herein, the LNP comprises about 1 mol % to about 4.5 mol % PEG lipid. In one embodiment of the LNPs or methods of the disclosure, the LNP comprises about 1 mol % to about 4 mol % PEG lipid. In one embodiment of the LNPs or methods of the disclosure, the LNP comprises about 1 mol % to about 3.5 mol % PEG lipid. In one embodiment of the LNPs or methods of the disclosure, the LNP comprises about 1 mol % to about 3 mol % PEG lipid. In one embodiment of the LNPs or methods of the disclosure, the LNP comprises about 1 mol % to about 2.5 mol % PEG lipid. In one embodiment of the LNPs or methods of the disclosure, the LNP comprises about 1 mol % to about 2 mol % PEG lipid. In one embodiment of the LNPs or methods of the disclosure, the LNP comprises about 1 mol % to about 1.5 mol % PEG lipid.

In an embodiment of any of the LNP compositions, methods or compositions for use disclosed herein, the LNP comprises about 1.5 mol % to about 5 mol % PEG lipid. In one embodiment of the LNPs or methods of the disclosure, the LNP comprises about 2 mol % to about 5 mol % PEG lipid. In one embodiment of the LNPs or methods of the disclosure, the LNP comprises about 2.5 mol % to about 5 mol % PEG lipid. In one embodiment of the LNPs or methods of the disclosure, the LNP comprises about 3 mol % to about 5 mol % PEG lipid. In one embodiment of the LNPs or methods of the disclosure, the LNP comprises about 3.5 mol % to about 5 mol % PEG lipid. In one embodiment of the LNPs or methods of the disclosure, the LNP comprises about 4 mol % to about 5 mol % PEG lipid. In one embodiment of the LNPs or methods of the disclosure, the LNP comprises about 4.5 mol % to about 5 mol % PEG lipid.

In one embodiment of the LNPs or methods of the disclosure, the LNP comprises about 1 mol % to about 2 mol % PEG lipid. In one embodiment of the LNPs or methods of the disclosure, the LNP comprises about 1.5 mol % to about 2.5 mol % PEG lipid. In one embodiment of the LNPs or methods of the disclosure, the LNP comprises about 2 mol % to about 3 mol % PEG lipid. In one embodiment of the LNPs or methods of the disclosure, the LNP comprises about 3.5 mol % to about 4.5 mol % PEG lipid. In one embodiment of the LNPs or methods of the disclosure, the LNP comprises about 4 mol % to about 5 mol % PEG lipid.

In an embodiment of any of the LNP compositions, methods or compositions for use disclosed herein, the LNP comprises about 1 mol % PEG lipid. In one embodiment of the LNPs or methods of the disclosure, the LNP comprises about 1.5 mol % PEG lipid. In one embodiment of the LNPs or methods of the disclosure, the LNP comprises about 2 mol % PEG lipid. In one embodiment of the LNPs or methods of the disclosure, the LNP comprises about 2.5 mol % PEG lipid. In one embodiment of the LNPs or methods of the disclosure, the LNP comprises about 3 mol % PEG lipid. In one embodiment of the LNPs or methods of the disclosure, the LNP comprises about 3.5 mol % PEG lipid. In one embodiment of the LNPs or methods of the disclosure, the LNP comprises about 4 mol % PEG lipid. In one embodiment of the LNPs or methods of the disclosure, the LNP comprises about 4.5 mol % PEG lipid. In one embodiment of the LNPs or methods of the disclosure, the LNP comprises about 5 mol % PEG lipid.

In one embodiment, the mol % sterol or other structural lipid is 18.5% phytosterol and the total mol % structural lipid is 38.5%. In one embodiment, the mol % sterol or other structural lipid is 28.5% phytosterol and the total mol % structural lipid is 38.5%.

In one embodiment of the LNPs, or methods of the disclosure, the LNP comprises about 50 mol % a compound of Formula (IIa) and about 10 mol % non-cationic helper lipid or phospholipid. In one embodiment of the LNPs or methods of the disclosure, the LNP comprises 50 mol % a compound of Formula (IIa) and about 10 mol % non-cationic helper lipid or phospholipid. In one embodiment of the LNPs, or methods of the disclosure, the LNP comprises about 50 mol % a compound of Formula (IIa) and 10 mol % non-cationic helper lipid or phospholipid. In one embodiment of the LNPs or methods of the disclosure, the LNP comprises 50 mol % a compound of Formula (IIa) and 10 mol % non-cationic helper lipid or phospholipid. In one embodiment of the LNPs or methods of the disclosure, the LNP comprises about 49.83 mol % a compound of Formula (IIa), about 9.83 mol % non-cationic helper lipid or phospholipid, about 30.33 mol % sterol or other structural lipid, and about 2.0 mol % PEG lipid.

In one embodiment of the LNPs, or methods of the disclosure, the LNP comprises about 50 mol % a compound of Formula (IIe) and about 10 mol % non-cationic helper lipid or phospholipid. In one embodiment of the LNPs or methods of the disclosure, the LNP comprises 50 mol % a compound of Formula (IIe) and about 10 mol % non-cationic helper lipid or phospholipid. In one embodiment of the LNPs or methods of the disclosure, the LNP comprises about 50 mol % a compound of Formula (IIe) and 10 mol % non-cationic helper lipid or phospholipid. In one embodiment of the LNPs or methods of the disclosure, the LNP comprises 50 mol % a compound of Formula (IIe) and 10 mol % non-cationic helper lipid or phospholipid. In one embodiment of the LNPs or methods of the disclosure, the LNP comprises about 49.83 mol % a compound of Formula (IIe), about 9.83 mol % non-cationic helper lipid or phospholipid, about 30.33 mol % sterol or other structural lipid, and about 2.0 mol % PEG lipid.

In an embodiment of any of the LNP compositions, methods or compositions for use disclosed herein, the LNP is formulated for intravenous, subcutaneous, intramuscular, intraocular, intranasal, rectal, pulmonary or oral delivery. In an embodiment, the LNP is formulated for intravenous delivery. In an embodiment, the LNP is formulated for subcutaneous delivery. In an embodiment, the LNP is formulated for intramuscular delivery. In an embodiment, the LNP is formulated for intraocular delivery. In an embodiment, the LNP is formulated for intranasal delivery. In an embodiment, the LNP is formulated for rectal delivery. In an embodiment, the LNP is formulated for pulmonary delivery. In an embodiment, the LNP is formulated for oral delivery.

In an embodiment of any of the methods or compositions for use disclosed herein, the subject is a mammal, e.g., a human.

Additional features of any of the aforesaid LNP compositions or methods of using said LNP compositions, include one or more of the following enumerated embodiments. Those skilled in the art will recognize or be able to ascertain using no more than routine experimentation, many equivalents to the specific embodiments of the invention described herein. Such equivalents are intended to be encompassed by the following enumerated embodiments.

### OTHER EMBODIMENTS OF THE DISCLOSURE

1. A polynucleotide encoding a polypeptide, wherein the polynucleotide comprises:

(a) a 5′-UTR comprising the sequence of SEQ ID NO: 1 or a variant or fragment thereof,

(b) a coding region comprising a stop element (e.g., as described herein); and

(c) a 3′-UTR (e.g., as described herein).

2. The polynucleotide of embodiment 1, wherein the 5′ UTR comprises a nucleic acid sequence of Formula A:

(SEQ ID NO: 46)
G G A A A U C G C A A A A (N₂)ₓ(N₃)ₓ
C U (N₄)ₓ(N₅)ₓ C G C G U U A G A U U
U C U U A G U U U U C U N₆ N₇ C A A C
U A G C A A G C U U U U U G U U C U
C G C C (N₈ C C)ₓ,

wherein:

(N₂)ₓ is a uracil and x is an integer from 0 to 5, e.g., wherein x=3 or 4;

(N₃)ₓ is a guanine and x is an integer from 0 to 1;

(N₄)ₓ is a cytosine and x is an integer from 0 to 1;

(N₅)ₓ is a uracil and x is an integer from 0 to 5, e.g., wherein x=2 or 3;

N₆ is a uracil or cytosine;

N₇ is a uracil or guanine; and/or

N₈ is a adenine or guanine and x is an integer from 0 to 1.

3. The polynucleotide of embodiment 1, wherein the variant of SEQ ID NO: 1 comprises a sequence with at least 50%, 60%, 70%, 80%, 85%, 90%, 95%, 96%, 97%, 98%, 99% or 100% identity to SEQ ID NO: 1, or a fragment thereof (e.g., a fragment that lacks the first one, two, three, four, five, or six nucleotides of SEQ ID NO: 1).
4. The polynucleotide of embodiment 1 or 3, wherein the variant of SEQ ID NO: 1 comprises a uridine content of at least 30%, 40%, 50%, 60%, 70%, or 80%.
5. The polynucleotide of any one of embodiments 1, or 3-4, wherein the variant of SEQ ID NO: 1 comprises at least 2, 3, 4, 5, 6, 7, 8, 9, 10, 11 or 12 consecutive uridines (e.g., a polyuridine tract).
6. The polynucleotide of embodiments 5, wherein the polyuridine tract in the variant of SEQ ID NO: 1 comprises at least 2-12, 2-11, 2-10, 2-9, 2-8, 2-7, 2-6, 2-5, 2-4, 2-3, 3-12, 4-12, 5-12, 6-12, 7-12, 8-12, 9-12, 10-12, 11-12, 2-6, or 3-5 consecutive uridines.
7. The polynucleotide of any one of embodiments 1 or 3-6, wherein the polyuridine tract in the variant of SEQ ID NO: 1 comprises 4 consecutive uridines.
8. The polynucleotide of any one of embodiments 1 or 3-7, wherein the polyuridine tract in the variant of SEQ ID NO: 1 comprises 5 consecutive uridines.
9. The polynucleotide of any one of embodiments 1 or 3-8, wherein the variant of SEQ ID NO: 1 comprises 1, 2, 3, 4, 5, 6, 7, 8, 9, 10, 11, 12, 13, 14, or 15 polyuridine tracts.
10. The polynucleotide of embodiment 9, wherein the variant of SEQ ID NO: 1 comprises 3 polyuridine tracts.
11. The polynucleotide of embodiment 9, wherein the variant of SEQ ID NO: 1 comprises 4 polyuridine tracts.
12. The polynucleotide of embodiment 9, wherein the variant of SEQ ID NO: 1 comprises 5 polyuridine tracts.
13. The polynucleotide of any one of embodiments 1 or 3-12, wherein one or more of the polyuridine tracts are adjacent to a different polyuridine tract.
14. The polynucleotide of any one of embodiments 1 or 3-13, wherein each of, e.g., all, the polyuridine tracts are adjacent to each other, e.g., all of the polyuridine tracts are contiguous.
15. The polynucleotide of any one of embodiments 1 or 3-14, wherein one or more of the polyuridine tracts are separated by 1, 2, 3, 4, 5, 6, 7, 8, 9, 10, 11, 2, 13, 14, 15, 16, 17, 18. 19, 20, 30, 40, 50 or 60 nucleotides.
16. The polynucleotide of any one of embodiments 1 or 3-15, wherein each of, e.g., all of, the polyuridine tracts are separated by 1, 2, 3, 4, 5, 6, 7, 8, 9, 10, 11, 2, 13, 14, 15, 16, 17, 18. 19, 20, 30, 40, 50 or 60 nucleotides.
17. The polynucleotide of any one of embodiments 1 or 3-16, wherein a first polyuridine tract and a second polyuridine tract are adjacent to each other.
18. The polynucleotide of embodiment 17, wherein a subsequent, e.g., third, fourth, fifth, sixth or seventh, eighth, ninth, or tenth, polyuridine tract is separated by 1, 2, 3, 4, 5, 6, 7, 8, 9, 10, 11, 2, 13, 14, 15, 16, 17, 18. 19, 20, 30, 40, 50 or 60 nucleotides from the first polyuridine tract, the second polyuridine tract, or any one of the subsequent polyuridine tracts.
19. The polynucleotide of any one of embodiments 1 or 3-18, wherein a first polyuridine tract is separated by 1, 2, 3, 4, 5, 6, 7, 8, 9, 10, 11, 2, 13, 14, 15, 16, 17, 18. 19, 20, 30, 40, 50 or 60 nucleotides from a subsequent polyuridine tract, e.g., a second, third, fourth, fifth, sixth or seventh, eighth, ninth, or tenth polyuridine tract.
20. The polynucleotide of embodiment 19, wherein one or more of the subsequent polyuridine tracts are adjacent to a different polyuridine tract.
21. The polynucleotide of any one of the preceding embodiments wherein the 5′ UTR comprises a Kozak sequence, e.g., a GCCRCC nucleotide sequence (SEQ ID NO: 43) wherein R is an adenine or guanine.
22. The polynucleotide of embodiment 21, wherein the Kozak sequence is disposed at the 3′ end of the 5′ UTR sequence.
23. The polynucleotide of any one of the preceding embodiments, wherein the 5′ UTR comprises the sequence of SEQ ID NO: 1, or a sequence with at least 80%, 85%, 90%, 95%, 96%, 97%, 98%, 99% or 100% identity to SEQ ID NO: 1, or a fragment thereof.
24. The polynucleotide of embodiment 23, wherein the 5′ UTR comprises the sequence of SEQ ID NO: 1.
25. The polynucleotide of any one of the preceding embodiments, wherein the 5′ UTR comprises the sequence of SEQ ID NO: 41, or a sequence with at least 80%, 85%, 90%, 95%, 96%, 97%, 98%, 99% or 100% identity to SEQ ID NO: 41, or a fragment thereof (e.g., a fragment that lacks the first one, two, three, four, five, or six nucleotides of SEQ ID NO: 41).
26. The polynucleotide of embodiment 25, wherein the 5′ UTR comprises the sequence of SEQ ID NO: 41 or a fragment thereof that lacks the first one, two, three, four, five, or six nucleotides of SEQ ID NO: 41.
27. The polynucleotide of any one of the preceding embodiments, wherein the 5′ UTR comprises the sequence of SEQ ID NO: 42, or a sequence with at least 80%, 85%, 90%, 95%, 96%, 97%, 98%, 99% or 100% identity to SEQ ID NO: 42, or a fragment thereof (e.g., a fragment that lacks the first one, two, three, four, five, or six nucleotides of SEQ ID NO: 42).
28. The polynucleotide of embodiment 27, wherein the 5′ UTR comprises the sequence of SEQ ID NO: 42 or a fragment thereof that lacks the first one, two, three, four, five, or six nucleotides of SEQ ID NO: 42.
29. The polynucleotide of any one of the preceding embodiments, wherein the 5′ UTR results in an increased half-life of the polynucleotide, e.g., about 1.5-20-fold increase in half-life of the polynucleotide.
30. The polynucleotide of embodiment 29, wherein the increase in half-life of the polynucleotide is compared to an otherwise similar polynucleotide which does not have a 5′ UTR, has a different 5′ UTR, or does not have a 5′ UTR of any one of embodiments 1-28.
31. The polynucleotide of any one of embodiments 29-30, wherein the increase in half-life of the polynucleotide is measured according to an assay that measures the half-life of a polynucleotide, e.g., an assay described in any one of Examples disclosed herein.
32. The polynucleotide of any one of the preceding embodiments, wherein the 5′ UTR results in an increased level and/or activity, e.g., output, of the polypeptide encoded by the polynucleotide.
33. The polynucleotide of embodiment 32, wherein the 5′ UTR results in about 1.5-20-fold increase in level and/or activity, e.g., output, of the polypeptide encoded by the polynucleotide.
34. The polynucleotide of embodiment 32 or 33, wherein the increase in level and/or activity, e.g., output, of the polypeptide encoded by the polynucleotide is compared to an otherwise similar polynucleotide which does not have a 5′ UTR, has a different 5′ UTR, or does not have the 5′ UTR of any one of embodiments 1-28.
35. The polynucleotide of any one of embodiments 32-34, wherein the increase in level and/or activity, e.g., output, of the polypeptide encoded by the polynucleotide is measured according to an assay that measures the level and/or activity of a polypeptide, e.g., an assay described in any one of Examples disclosed herein.
36. The polynucleotide of embodiment 35, wherein the 5′ UTR results in an increase in activity of the polypeptide encoded by the polynucleotide, e.g., an increase of about 1.2-10-fold.
37. The polynucleotide of embodiment 36, wherein the increase in activity is compared to an otherwise similar polynucleotide which does not have a 5′ UTR, has a different 5′ UTR, or does not have the 5′ UTR of any one of embodiments 1-28.
38. The polynucleotide of embodiment 36 or 37, wherein the increase in level of the polypeptide encoded by the polynucleotide is measured according to an assay that measures the level of a polypeptide, e.g., an assay described in any one of Examples disclosed herein.
39. A polynucleotide encoding a polypeptide, wherein the polynucleotide comprises:

(a) a 5′-UTR comprising the sequence of SEQ ID NO: 2, SEQ ID NO: 3, SEQ ID NO: 4, SEQ ID NO: 5, SEQ ID NO: 6, SEQ ID NO: 8, SEQ ID NO: 41, SEQ ID NO: 42, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 88, SEQ ID NO: 89 or SEQ ID NO: 90, or a variant or a fragment thereof (e.g., a fragment that lacks the first one, two, three, four, five, or six nucleotides of any of the aforesaid sequences);

(b) a coding region comprising a stop element; and

(c) a 3′-UTR,

wherein the 5′ UTR comprises at least 2, 3, 4, 5, 6, 7, 8, 9, 10, 11, or 12 consecutive uridines (e.g., a polyuridine tract).

40. The polynucleotide of embodiment 39, wherein the 5′ UTR comprises a variant of SEQ ID NO: 2, SEQ ID NO: 3, SEQ ID NO: 4, SEQ ID NO: 5, SEQ ID NO: 6, or SEQ ID NO: 8, SEQ ID NO: 41, SEQ ID NO: 42, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 88, SEQ ID NO: 89 or SEQ ID NO: 90.
41. The polynucleotide of embodiment 40, wherein the 5′ UTR variant comprises a sequence with at least 50%, 60%, 70%, 80%, 85%, 90%, 95%, 96%, 97%, 98%, 99% or 100% identity to SEQ ID NO: 2, SEQ ID NO: 3, SEQ ID NO: 4, SEQ ID NO: 5, SEQ ID NO: 6, or SEQ ID NO: 8, SEQ ID NO: 41, SEQ ID NO: 42, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 88, SEQ ID NO: 89 or SEQ ID NO: 90, or a fragment thereof (e.g., a fragment that lacks the first one, two, three, four, five, or six nucleotides of any of the aforesaid sequences).
42. The polynucleotide of embodiment 40 or 41, wherein the 5′ UTR variant comprises a uridine content of at least 30%, 40%, 50%, 60%, 70%, or 80%.
43. The polynucleotide of any one of embodiments 40-42, wherein the 5′ UTR variant comprises at least 2, 3, 4, 5, 6, 7, 8, 9, 10, 11 or 12 consecutive uridines (e.g., a polyuridine tract).
44. The polynucleotide of embodiment 43, wherein the polyuridine tract in the 5′ UTR variant comprises at least 2-12, 2-11, 2-10, 2-9, 2-8, 2-7, 2-6, 2-5, 2-4, 2-3, 3-12, 4-12, 5-12, 6-12, 7-12, 8-12, 9-12, 10-12, 11-12, 2-6, or 3-5 consecutive uridines.
45. The polynucleotide of any one of embodiments 40-44, wherein the polyuridine tract in the 5′ UTR variant comprises 4 consecutive uridines.
46. The polynucleotide of any one of embodiments 40-44, wherein the polyuridine tract in the 5′ UTR variant comprises 5 consecutive uridines.
47. The polynucleotide of any one of embodiments 40-46, wherein the 5′ UTR variant comprises 1, 2, 3, 4, 5, 6, 7, 8, 9, 10, 11, 12, 13, 14, or 15 polyuridine tracts.
48. The polynucleotide of embodiment 47, wherein the 5′ UTR variant comprises 3 polyuridine tracts.
49. The polynucleotide of embodiment 47, wherein the 5′ UTR variant comprises 4 polyuridine tracts.
50. The polynucleotide of embodiment 47, wherein the 5′ UTR variant comprises 5 polyuridine tracts.
51. The polynucleotide of any one of embodiments 40-50, wherein one or more of the polyuridine tracts are adjacent to a different polyuridine tract.
52. The polynucleotide of any one of embodiments 40-51, wherein each of, e.g., all, the polyuridine tracts are adjacent to each other, e.g., all of the polyuridine tracts are contiguous.
53. The polynucleotide of any one of embodiments 40-52, wherein one or more of the polyuridine tracts are separated by 1, 2, 3, 4, 5, 6, 7, 8, 9, 10, 11, 2, 13, 14, 15, 16, 17, 18. 19, 20, 30, 40, 50 or 60 nucleotides.
54. The polynucleotide of any one of embodiments 40-53, wherein each of, e.g., all of, the polyuridine tracts are separated by 1, 2, 3, 4, 5, 6, 7, 8, 9, 10, 11, 2, 13, 14, 15, 16, 17, 18. 19, 20, 30, 40, 50 or 60 nucleotides.
55. The polynucleotide of any one of embodiments 40-54, wherein a first polyuridine tract and a second polyuridine tract are adjacent to each other.
56. The polynucleotide of embodiments 55, wherein a subsequent, e.g., third, fourth, fifth, sixth or seventh, eighth, ninth, or tenth, polyuridine tract is separated by 1, 2, 3, 4, 5, 6, 7, 8, 9, 10, 11, 2, 13, 14, 15, 16, 17, 18. 19, 20, 30, 40, 50 or 60 nucleotides from the first polyuridine tract, the second polyuridine tract, or any one of the subsequent polyuridine tracts.
57. The polynucleotide of any one of embodiments 40-56, wherein a first polyuridine tract is separated by 1, 2, 3, 4, 5, 6, 7, 8, 9, 10, 11, 2, 13, 14, 15, 16, 17, 18. 19, 20, 30, 40, 50 or 60 nucleotides from a subsequent polyuridine tract, e.g., a second, third, fourth, fifth, sixth or seventh, eighth, ninth, or tenth polyuridine tract.
58. The polynucleotide of embodiment 57, wherein one or more of the subsequent polyuridine tracts are adjacent to a different polyuridine tract.
59. The polynucleotide of any one of embodiments, 39-58, wherein the 5′ UTR comprises a Kozak sequence, e.g., a GCCRCC nucleotide sequence (SEQ ID NO: 43) wherein R is an adenine or guanine.
60. The polynucleotide of embodiment 59, wherein the Kozak sequence is disposed at the 3′ end of the 5′ UTR sequence.
61. The polynucleotide of any one of embodiments 39-60, wherein the 5′ UTR comprises the sequence of SEQ ID NO: 2 or a fragment thereof (e.g., a fragment that lacks the first one, two, three, four, five, or six nucleotides of SEQ ID NO: 2).
62. The polynucleotide of any one of embodiments 39-60, wherein the 5′ UTR comprises the sequence of SEQ ID NO: 3 or a fragment thereof (e.g., a fragment that lacks the first one, two, three, four, five, or six nucleotides of SEQ ID NO: 3).
63. The polynucleotide of any one of embodiments 39-60, wherein the 5′ UTR comprises the sequence of SEQ ID NO: 4 or a fragment thereof (e.g., a fragment that lacks the first one, two, three, four, five, or six nucleotides of SEQ ID NO: 4).
64. The polynucleotide of any one of embodiments 39-60, wherein the 5′ UTR comprises the sequence of SEQ ID NO: 5 or a fragment thereof (e.g., a fragment that lacks the first one, two, three, four, five, or six nucleotides of SEQ ID NO: 5).
65. The polynucleotide of any one of embodiments 39-60, wherein the 5′ UTR comprises the sequence of SEQ ID NO: 6 or a fragment thereof (e.g., a fragment that lacks the first one, two, three, four, five, or six nucleotides of SEQ ID NO: 6).
66. The polynucleotide of any one of embodiments 39-60, wherein the 5′ UTR comprises the sequence of SEQ ID NO: 8 or a fragment thereof (e.g., a fragment that lacks the first one, two, three, four, five, or six nucleotides of SEQ ID NO: 8).
67. The polynucleotide of any one of embodiments 39-60, wherein the 5′ UTR comprises the sequence of SEQ ID NO: 41 or a fragment thereof (e.g., a fragment that lacks the first one, two, three, four, five, or six nucleotides of SEQ ID NO: 41).
68. The polynucleotide of any one of embodiments 39-60, wherein the 5′ UTR comprises the sequence of SEQ ID NO: 42 or a fragment thereof (e.g., a fragment that lacks the first one, two, three, four, five, or six nucleotides of SEQ ID NO: 42).
69. The polynucleotide of any one of embodiments 39-60, wherein the 5′ UTR comprises the sequence of SEQ ID NO: 63 or a fragment thereof (e.g., a fragment that lacks the first one, two, three, four, five, or six nucleotides of SEQ ID NO: 63).
70. The polynucleotide of any one of embodiments 39-60, wherein the 5′ UTR comprises the sequence of SEQ ID NO: 64 or a fragment thereof (e.g., a fragment that lacks the first one, two, three, four, five, or six nucleotides of SEQ ID NO: 64).
71. The polynucleotide of any one of embodiments 39-60, wherein the 5′ UTR comprises the sequence of SEQ ID NO: 65 or a fragment thereof (e.g., a fragment that lacks the first one, two, three, four, five, or six nucleotides of SEQ ID NO: 65).
72. The polynucleotide of any one of embodiments 39-60, wherein the 5′ UTR comprises the sequence of SEQ ID NO: 66 or a fragment thereof (e.g., a fragment that lacks the first one, two, three, four, five, or six nucleotides of SEQ ID NO: 66).
73. The polynucleotide of any one of embodiments 39-60, wherein the 5′ UTR comprises the sequence of SEQ ID NO: 67 or a fragment thereof (e.g., a fragment that lacks the first one, two, three, four, five, or six nucleotides of SEQ ID NO: 67).
74. The polynucleotide of any one of embodiments 39-60, wherein the 5′ UTR comprises the sequence of SEQ ID NO: 68 or a fragment thereof (e.g., a fragment that lacks the first one, two, three, four, five, or six nucleotides of SEQ ID NO: 68).
75. The polynucleotide of any one of embodiments 39-60, wherein the 5′ UTR comprises the sequence of SEQ ID NO: 69 or a fragment thereof (e.g., a fragment that lacks the first one, two, three, four, five, or six nucleotides of SEQ ID NO: 69).
76. The polynucleotide of any one of embodiments 39-60, wherein the 5′ UTR comprises the sequence of SEQ ID NO: 70 or a fragment thereof (e.g., a fragment that lacks the first one, two, three, four, five, or six nucleotides of SEQ ID NO: 70).
77. The polynucleotide of any one of embodiments 39-60, wherein the 5′ UTR comprises the sequence of SEQ ID NO: 70 or a fragment thereof (e.g., a fragment that lacks the first one, two, three, four, five, or six nucleotides of SEQ ID NO: 70).
78. The polynucleotide of any one of embodiments 39-60, wherein the 5′ UTR comprises the sequence of SEQ ID NO: 71 or a fragment thereof (e.g., a fragment that lacks the first one, two, three, four, five, or six nucleotides of SEQ ID NO: 71).
79. The polynucleotide of any one of embodiments 39-60, wherein the 5′ UTR comprises the sequence of SEQ ID NO: 72 or a fragment thereof (e.g., a fragment that lacks the first one, two, three, four, five, or six nucleotides of SEQ ID NO: 72).
80. The polynucleotide of any one of embodiments 39-60, wherein the 5′ UTR comprises the sequence of SEQ ID NO: 73 or a fragment thereof (e.g., a fragment that lacks the first one, two, three, four, five, or six nucleotides of SEQ ID NO: 73).
81. The polynucleotide of any one of embodiments 39-60, wherein the 5′ UTR comprises the sequence of SEQ ID NO: 74 or a fragment thereof (e.g., a fragment that lacks the first one, two, three, four, five, or six nucleotides of SEQ ID NO: 74).
82. The polynucleotide of any one of embodiments 39-60, wherein the 5′ UTR comprises the sequence of SEQ ID NO: 75 or a fragment thereof (e.g., a fragment that lacks the first one, two, three, four, five, or six nucleotides of SEQ ID NO: 75).
83. The polynucleotide of any one of embodiments 39-60, wherein the 5′ UTR comprises the sequence of SEQ ID NO: 76 or a fragment thereof (e.g., a fragment that lacks the first one, two, three, four, five, or six nucleotides of SEQ ID NO: 76).
84. The polynucleotide of any one of embodiments 39-60, wherein the 5′ UTR comprises the sequence of SEQ ID NO: 77 or a fragment thereof (e.g., a fragment that lacks the first one, two, three, four, five, or six nucleotides of SEQ ID NO: 77).
85. The polynucleotide of any one of embodiments 39-60, wherein the 5′ UTR comprises the sequence of SEQ ID NO: 78 or a fragment thereof (e.g., a fragment that lacks the first one, two, three, four, five, or six nucleotides of SEQ ID NO: 78).
86. The polynucleotide of any one of embodiments 39-60, wherein the 5′ UTR comprises the sequence of SEQ ID NO: 88 or a fragment thereof (e.g., a fragment that lacks the first one, two, three, four, five, or six nucleotides of SEQ ID NO: 88).
87. The polynucleotide of any one of embodiments 39-60, wherein the 5′ UTR comprises the sequence of SEQ ID NO: 89 or a fragment thereof (e.g., a fragment that lacks the first one, two, three, four, five, or six nucleotides of SEQ ID NO: 89).
88. The polynucleotide of any one of embodiments 39-60, wherein the 5′ UTR comprises the sequence of SEQ ID NO: 90 or a fragment thereof (e.g., a fragment that lacks the first one, two, three, four, five, or six nucleotides of SEQ ID NO: 90).
89. The polynucleotide of any one of embodiments 39-88, wherein the 5′ UTR results in about 1.5-20-fold increase in half-life of the polynucleotide.
90. The polynucleotide of embodiment 89, wherein the 5′ UTR results in an increased level and/or activity, e.g., output, of the polypeptide encoded by the polynucleotide.
91. The polynucleotide of any one of the preceding embodiments, wherein the coding region of (b) comprises a stop element chosen from a stop element provided in Table 3, e.g., SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO:36, SEQ ID NO; 62, SEQ ID NO: 93 or SEQ ID NO: 96.
92. The polynucleotide of embodiment 91, wherein the stop element comprises the sequence of SEQ ID NO: 26.
93. The polynucleotide of embodiment 91, wherein the stop element comprises the sequence of SEQ ID NO: 27.
94. The polynucleotide of embodiment 91, wherein the stop element comprises the sequence of SEQ ID NO: 28.
95. The polynucleotide of embodiment 91, wherein the stop element comprises the sequence of SEQ ID NO: 29.
96. The polynucleotide of embodiment 91, wherein the stop element comprises the sequence of SEQ ID NO: 30.
97. The polynucleotide of embodiment 91, wherein the stop element comprises the sequence of SEQ ID NO: 31.
98. The polynucleotide of embodiment 91, wherein the stop element comprises the sequence of SEQ ID NO: 32.
99. The polynucleotide of embodiment 91, wherein the stop element comprises the sequence of SEQ ID NO: 33.
100. The polynucleotide of embodiment 91, wherein the stop element comprises the sequence of SEQ ID NO: 34.
101. The polynucleotide of embodiment 91, wherein the stop element comprises the sequence of SEQ ID NO: 35.
102. The polynucleotide of embodiment 91, wherein the stop element comprises the sequence of SEQ ID NO: 36.
103. The polynucleotide of embodiment 91, wherein the stop element comprises the sequence of SEQ ID NO: 62.
104. The polynucleotide of embodiment 91, wherein the stop element comprises the sequence of SEQ ID NO: 93.
105. The polynucleotide of embodiment 91, wherein the stop element comprises the sequence of SEQ ID NO: 96.
106. The polynucleotide of embodiment 91, wherein the coding region of (b) comprises a stop element comprising the consensus sequence of SEQ ID NO: 37, SEQ ID NO: 56 or SEQ ID NO: 57.
107. The polynucleotide of any one of embodiments 1-90, wherein the 3′ UTR of (c) comprises a 3′ UTR sequence provided in Table 2 or a sequence with at least 80%, 85%, 90%, 95%, 96%, 97%, 98%, 99% or 100% identity to a 3′ UTR sequence provided in Table 2, or a fragment thereof (e.g., a fragment that lacks the first one, two, three, four, five, six, or more nucleotides of the 3′ UTR sequence provided in Table 2),

optionally wherein the polynucleotide comprises a sequence with at least 80%, 85%, 90%, 95%, 96%, 97%, 98%, 99% or 100% identity to a sequence provided in Table 4.

108. The polynucleotide of embodiment 107, wherein the 3′ UTR comprises a sequence with at least 80%, 85%, 90%, 95%, 96%, 97%, 98%, 99% or 100% identity to SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 45, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 94 or SEQ ID NO: 95, or a fragment thereof (e.g., a fragment that lacks the first one, two, three, four, five, six, or more nucleotides of any of the aforesaid sequences),

optionally wherein the polynucleotide comprises a sequence with at least 80%, 85%, 90%, 95%, 96%, 97%, 98%, 99% or 100% identity to any of SEQ ID NOs: 47, 48, 49, 50, 122, 52, 53, 54, 55, 59, 60, 61, 126, 127, 97, 98, 99, 100, 101, 102, 103, 104, 105, 106, 107, 108, 109, 110, 111, 112, 113, 114, 115, 116, 117, 118, 119, or 120, or a variant or fragment thereof.

109. The polynucleotide of embodiment 107 or 108, wherein the 3′ UTR comprises the sequence of SEQ ID NO: 11, or a sequence with at least 80%, 85%, 90%, 95%, 96%, 97%, 98%, 99% or 100% identity to SEQ ID NO: 11, or a fragment thereof (e.g., a fragment that lacks the first one, two, three, four, five, six, or more nucleotides of SEQ ID NO: 11).
110. The polynucleotide of embodiment 107 or 108, wherein the 3′ UTR comprises the sequence of SEQ ID NO: 12, or a sequence with at least 80%, 85%, 90%, 95%, 96%, 97%, 98%, 99% or 100% identity to SEQ ID NO: 12, or a fragment thereof (e.g., a fragment that lacks the first one, two, three, four, five, six, or more nucleotides of SEQ ID NO: 12).
111. The polynucleotide of embodiment 107 or 108, wherein the 3′ UTR comprises the sequence of SEQ ID NO: 13, or a sequence with at least 80%, 85%, 90%, 95%, 96%, 97%, 98%, 99% or 100% identity to SEQ ID NO: 13, or a fragment thereof (e.g., a fragment that lacks the first one, two, three, four, five, six, or more nucleotides of SEQ ID NO: 13).
112. The polynucleotide of embodiment 107 or 108, wherein the 3′ UTR comprises the sequence of SEQ ID NO: 14, or a sequence with at least 80%, 85%, 90%, 95%, 96%, 97%, 98%, 99% or 100% identity to SEQ ID NO: 14, or a fragment thereof (e.g., a fragment that lacks the first one, two, three, four, five, six, or more nucleotides of SEQ ID NO: 14).
113. The polynucleotide of embodiment 107 or 108, wherein the 3′ UTR comprises the sequence of SEQ ID NO: 15, or a sequence with at least 80%, 85%, 90%, 95%, 96%, 97%, 98%, 99% or 100% identity to SEQ ID NO: 15, or a fragment thereof (e.g., a fragment that lacks the first one, two, three, four, five, six, or more nucleotides of SEQ ID NO: 15).
114. The polynucleotide of embodiment 107 or 108, wherein the 3′ UTR comprises the sequence of SEQ ID NO: 16, or a sequence with at least 80%, 85%, 90%, 95%, 96%, 97%, 98%, 99% or 100% identity to SEQ ID NO: 16, or a fragment thereof (e.g., a fragment that lacks the first one, two, three, four, five, six, or more nucleotides of SEQ ID NO: 16).
115. The polynucleotide of embodiment 107 or 108, wherein the 3′ UTR comprises the sequence of SEQ ID NO: 17, or a sequence with at least 80%, 85%, 90%, 95%, 96%, 97%, 98%, 99% or 100% identity to SEQ ID NO: 17, or a fragment thereof (e.g., a fragment that lacks the first one, two, three, four, five, six, or more nucleotides of SEQ ID NO: 17).
116. The polynucleotide of embodiment 107 or 108, wherein the 3′ UTR comprises the sequence of SEQ ID NO: 18, or a sequence with at least 80%, 85%, 90%, 95%, 96%, 97%, 98%, 99% or 100% identity to SEQ ID NO: 18, or a fragment thereof (e.g., a fragment that lacks the first one, two, three, four, five, six, or more nucleotides of SEQ ID NO: 18).
117. The polynucleotide of embodiment 107 or 108, wherein the 3′ UTR comprises the sequence of SEQ ID NO: 19, or a sequence with at least 80%, 85%, 90%, 95%, 96%, 97%, 98%, 99% or 100% identity to SEQ ID NO: 19, or a fragment thereof (e.g., a fragment that lacks the first one, two, three, four, five, six, or more nucleotides of SEQ ID NO: 19).
118. The polynucleotide of embodiment 107 or 108, wherein the 3′ UTR comprises the sequence of SEQ ID NO: 21, or a sequence with at least 80%, 85%, 90%, 95%, 96%, 97%, 98%, 99% or 100% identity to SEQ ID NO: 20, or a fragment thereof (e.g., a fragment that lacks the first one, two, three, four, five, six, or more nucleotides of SEQ ID NO: 20).
119. The polynucleotide of embodiment 107 or 108, wherein the 3′ UTR comprises the sequence of SEQ ID NO: 21, or a sequence with at least 80%, 85%, 90%, 95%, 96%, 97%, 98%, 99% or 100% identity to SEQ ID NO: 21, or a fragment thereof (e.g., a fragment that lacks the first one, two, three, four, five, six, or more nucleotides of SEQ ID NO: 21).
120. The polynucleotide of embodiment 107 or 108, wherein the 3′ UTR comprises the sequence of SEQ ID NO: 22, or a sequence with at least 80%, 85%, 90%, 95%, 96%, 97%, 98%, 99% or 100% identity to SEQ ID NO: 22, or a fragment thereof (e.g., a fragment that lacks the first one, two, three, four, five, six, or more nucleotides of SEQ ID NO: 22).
121. The polynucleotide of embodiment 107 or 108, wherein the 3′ UTR comprises the sequence of SEQ ID NO: 23, or a sequence with at least 80%, 85%, 90%, 95%, 96%, 97%, 98%, 99% or 100% identity to SEQ ID NO: 23, or a fragment thereof (e.g., a fragment that lacks the first one, two, three, four, five, six, or more nucleotides of SEQ ID NO: 23).
122. The polynucleotide of embodiment 107 or 108, wherein the 3′ UTR comprises the sequence of SEQ ID NO: 24, or a sequence with at least 80%, 85%, 90%, 95%, 96%, 97%, 98%, 99% or 100% identity to SEQ ID NO: 24, or a fragment thereof (e.g., a fragment that lacks the first one, two, three, four, five, six, or more nucleotides of SEQ ID NO: 24).
123. The polynucleotide of embodiment 107 or 108, wherein the 3′ UTR comprises the sequence of SEQ ID NO: 25, or a sequence with at least 80%, 85%, 90%, 95%, 96%, 97%, 98%, 99% or 100% identity to SEQ ID NO: 25, or a fragment thereof (e.g., a fragment that lacks the first one, two, three, four, five, six, or more nucleotides of SEQ ID NO: 25).
124. The polynucleotide of embodiment 107 or 108, wherein the 3′ UTR comprises the sequence of SEQ ID NO: 45, or a sequence with at least 80%, 85%, 90%, 95%, 96%, 97%, 98%, 99% or 100% identity to SEQ ID NO: 45, or a fragment thereof (e.g., a fragment that lacks the first one, two, three, four, five, six, or more nucleotides of SEQ ID NO: 45).
125. The polynucleotide of embodiment 107 or 108, wherein the 3′ UTR comprises the sequence of SEQ ID NO: 79, or a sequence with at least 80%, 85%, 90%, 95%, 96%, 97%, 98%, 99% or 100% identity to SEQ ID NO: 79, or a fragment thereof (e.g., a fragment that lacks the first one, two, three, four, five, six, or more nucleotides of SEQ ID NO: 79).
126. The polynucleotide of embodiment 107 or 108, wherein the 3′ UTR comprises the sequence of SEQ ID NO: 80, or a sequence with at least 80%, 85%, 90%, 95%, 96%, 97%, 98%, 99% or 100% identity to SEQ ID NO: 80, or a fragment thereof (e.g., a fragment that lacks the first one, two, three, four, five, six, or more nucleotides of SEQ ID NO: 80).
127. The polynucleotide of embodiment 107 or 108, wherein the 3′ UTR comprises the sequence of SEQ ID NO: 81, or a sequence with at least 80%, 85%, 90%, 95%, 96%, 97%, 98%, 99% or 100% identity to SEQ ID NO: 81, or a fragment thereof (e.g., a fragment that lacks the first one, two, three, four, five, six, or more nucleotides of SEQ ID NO: 81).
128. The polynucleotide of embodiment 107 or 108, wherein the 3′ UTR comprises the sequence of SEQ ID NO: 82, or a sequence with at least 80%, 85%, 90%, 95%, 96%, 97%, 98%, 99% or 100% identity to SEQ ID NO: 82, or a fragment thereof (e.g., a fragment that lacks the first one, two, three, four, five, six, or more nucleotides of SEQ ID NO: 82).
129. The polynucleotide of embodiment 107 or 108, wherein the 3′ UTR comprises the sequence of SEQ ID NO: 83, or a sequence with at least 80%, 85%, 90%, 95%, 96%, 97%, 98%, 99% or 100% identity to SEQ ID NO: 83, or a fragment thereof (e.g., a fragment that lacks the first one, two, three, four, five, six, or more nucleotides of SEQ ID NO: 83).
130. The polynucleotide of embodiment 107 or 108, wherein the 3′ UTR comprises the sequence of SEQ ID NO: 84, or a sequence with at least 80%, 85%, 90%, 95%, 96%, 97%, 98%, 99% or 100% identity to SEQ ID NO: 84, or a fragment thereof (e.g., a fragment that lacks the first one, two, three, four, five, six, or more nucleotides of SEQ ID NO: 84).
131. The polynucleotide of embodiment 107 or 108, wherein the 3′ UTR comprises the sequence of SEQ ID NO: 85, or a sequence with at least 80%, 85%, 90%, 95%, 96%, 97%, 98%, 99% or 100% identity to SEQ ID NO: 85, or a fragment thereof (e.g., a fragment that lacks the first one, two, three, four, five, six, or more nucleotides of SEQ ID NO: 85).
132. The polynucleotide of embodiment 107 or 108, wherein the 3′ UTR comprises the sequence of SEQ ID NO: 86, or a sequence with at least 80%, 85%, 90%, 95%, 96%, 97%, 98%, 99% or 100% identity to SEQ ID NO: 86, or a fragment thereof (e.g., a fragment that lacks the first one, two, three, four, five, six, or more nucleotides of SEQ ID NO: 86).
133. The polynucleotide of embodiment 107 or 108, wherein the 3′ UTR comprises the sequence of SEQ ID NO: 87, or a sequence with at least 80%, 85%, 90%, 95%, 96%, 97%, 98%, 99% or 100% identity to SEQ ID NO: 87, or a fragment thereof (e.g., a fragment that lacks the first one, two, three, four, five, six, or more nucleotides of SEQ ID NO: 87).
134. The polynucleotide of embodiment 107 or 108, wherein the 3′ UTR comprises the sequence of SEQ ID NO: 94, or a sequence with at least 80%, 85%, 90%, 95%, 96%, 97%, 98%, 99% or 100% identity to SEQ ID NO: 94, or a fragment thereof (e.g., a fragment that lacks the first one, two, three, four, five, six, or more nucleotides of SEQ ID NO: 94).
135. The polynucleotide of embodiment 107 or 108, wherein the 3′ UTR comprises the sequence of SEQ ID NO: 95, or a sequence with at least 80%, 85%, 90%, 95%, 96%, 97%, 98%, 99% or 100% identity to SEQ ID NO: 95, or a fragment thereof (e.g., a fragment that lacks the first one, two, three, four, five, six, or more nucleotides of SEQ ID NO: 95).
136. The polynucleotide of embodiment 107 or 108, wherein the 3′ UTR comprises a micro RNA (miRNA) binding site, e.g., as described herein; and/or a TENT recruiting sequence, e.g., as described herein.
137. The polynucleotide of embodiment 136, wherein the miRNA binding site comprises a sequence of any one of SEQ ID NOs: 38-40; and/or wherein the TENT recruiting sequence comprises a sequence of SEQ ID NO: 91 or 92.
138. The polynucleotide of any one of embodiments 1-90, wherein:

(i) the coding region of (b) comprises a stop element chosen from a stop element provided in Table 3, e.g., SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO:36, SEQ ID NO: 37, SEQ ID NO: 56, SEQ ID NO: 57, SEQ ID NO: 62, SEQ ID NO: 93 or SEQ ID NO: 96; and

(ii) the 3′ UTR of (c) comprises a 3′ UTR sequence provided in Table 2 or a sequence with at least 80%, 85%, 90%, 95%, 96%, 97%, 98%, 99% or 100% identity to a 3′ UTR sequence provided in Table 2, or a fragment thereof (e.g., a fragment that lacks the first one, two, three, four, five, six, or more nucleotides of the 3′ UTR sequence provided in Table 2),

optionally wherein the polynucleotide comprises a sequence with at least 80%, 85%, 90%, 95%, 96%, 97%, 98%, 99% or 100% identity to a sequence provided in Table 4, e.g., any of SEQ ID NOs: 47, 48, 49, 50, 122, 52, 53, 54, 55, 59, 60, 61, 126, 127, 97, 98, 99, 100, 101, 102, 103, 104, 105, 106, 107, 108, 109, 110, 111, 112, 113, 114, 115, 116, 117, 118, 119, or 120, or a variant or fragment thereof.

139. The polynucleotide of embodiment 138, wherein the stop element comprises the sequence of SEQ ID NO: 26.
140. The polynucleotide of embodiment 138, wherein the stop element comprises the sequence of SEQ ID NO: 27.
141. The polynucleotide of embodiment 138, wherein the stop element comprises the sequence of SEQ ID NO: 28.
142. The polynucleotide of embodiment 138, wherein the stop element comprises the sequence of SEQ ID NO: 29.
143. The polynucleotide of embodiment 138, wherein the stop element comprises the sequence of SEQ ID NO: 30.
144. The polynucleotide of embodiment 138, wherein the stop element comprises the sequence of SEQ ID NO: 31.
145. The polynucleotide of embodiment 138, wherein the stop element comprises the sequence of SEQ ID NO: 32.
146. The polynucleotide of embodiment 138, wherein the stop element comprises the sequence of SEQ ID NO: 33.
147. The polynucleotide of embodiment 138, wherein the stop element comprises the sequence of SEQ ID NO: 34.
148. The polynucleotide of embodiment 138, wherein the stop element comprises the sequence of SEQ ID NO: 35.
149. The polynucleotide of embodiment 138, wherein the stop element comprises the sequence of SEQ ID NO: 36.
150. The polynucleotide of embodiment 138, wherein the stop element comprises the sequence of SEQ ID NO: 37.
151. The polynucleotide of embodiment 138, wherein the stop element comprises the sequence of SEQ ID NO: 56.
152. The polynucleotide of embodiment 138, wherein the stop element comprises the sequence of SEQ ID NO: 57.
153. The polynucleotide of embodiment 138, wherein the stop element comprises the sequence of SEQ ID NO: 62.
154. The polynucleotide of embodiment 138, wherein the stop element comprises the sequence of SEQ ID NO: 93.
155. The polynucleotide of embodiment 138, wherein the stop element comprises the sequence of SEQ ID NO: 96.
156. The polynucleotide of embodiment 139, wherein the coding region of (b) comprises a stop element comprising the consensus sequence of SEQ ID NO: 37.
157. The polynucleotide of any one of embodiments 139-156, wherein the 3′ UTR comprises the sequence of SEQ ID NO: 11, or a sequence with at least 80%, 85%, 90%, 95%, 96%, 97%, 98%, 99% or 100% identity to SEQ ID NO: 11, or a fragment thereof (e.g., a fragment that lacks the first one, two, three, four, five, six, or more nucleotides of SEQ ID NO: 11).
158. The polynucleotide of any one of embodiments 139-156, wherein the 3′ UTR comprises the sequence of SEQ ID NO: 12, or a sequence with at least 80%, 85%, 90%, 95%, 96%, 97%, 98%, 99% or 100% identity to SEQ ID NO: 12, or a fragment thereof (e.g., a fragment that lacks the first one, two, three, four, five, six, or more nucleotides of SEQ ID NO: 12).
159. The polynucleotide of any one of embodiments 139-156, wherein the 3′ UTR comprises the sequence of SEQ ID NO: 13, or a sequence with at least 80%, 85%, 90%, 95%, 96%, 97%, 98%, 99% or 100% identity to SEQ ID NO: 13, or a fragment thereof (e.g., a fragment that lacks the first one, two, three, four, five, six, or more nucleotides of SEQ ID NO: 13).
160. The polynucleotide of any one of embodiments 139-156, wherein the 3′ UTR comprises the sequence of SEQ ID NO: 14, or a sequence with at least 80%, 85%, 90%, 95%, 96%, 97%, 98%, 99% or 100% identity to SEQ ID NO: 14, or a fragment thereof (e.g., a fragment that lacks the first one, two, three, four, five, six, or more nucleotides of SEQ ID NO: 14).
161. The polynucleotide of any one of embodiments 139-156, wherein the 3′ UTR comprises the sequence of SEQ ID NO: 15, or a sequence with at least 80%, 85%, 90%, 95%, 96%, 97%, 98%, 99% or 100% identity to SEQ ID NO: 15, or a fragment thereof (e.g., a fragment that lacks the first one, two, three, four, five, six, or more nucleotides of SEQ ID NO: 15).
162. The polynucleotide of any one of embodiments 139-156, wherein the 3′ UTR comprises the sequence of SEQ ID NO: 16, or a sequence with at least 80%, 85%, 90%, 95%, 96%, 97%, 98%, 99% or 100% identity to SEQ ID NO: 16, or a fragment thereof (e.g., a fragment that lacks the first one, two, three, four, five, six, or more nucleotides of SEQ ID NO: 16).
163. The polynucleotide of any one of embodiments 139-156, wherein the 3′ UTR comprises the sequence of SEQ ID NO: 17, or a sequence with at least 80%, 85%, 90%, 95%, 96%, 97%, 98%, 99% or 100% identity to SEQ ID NO: 17, or a fragment thereof (e.g., a fragment that lacks the first one, two, three, four, five, six, or more nucleotides of SEQ ID NO: 17).
164. The polynucleotide of any one of embodiments 139-156, wherein the 3′ UTR comprises the sequence of SEQ ID NO: 18, or a sequence with at least 80%, 85%, 90%, 95%, 96%, 97%, 98%, 99% or 100% identity to SEQ ID NO: 18, or a fragment thereof (e.g., a fragment that lacks the first one, two, three, four, five, six, or more nucleotides of SEQ ID NO: 18).
165. The polynucleotide of any one of embodiments 139-156, wherein the 3′ UTR comprises the sequence of SEQ ID NO: 19, or a sequence with at least 80%, 85%, 90%, 95%, 96%, 97%, 98%, 99% or 100% identity to SEQ ID NO: 19, or a fragment thereof (e.g., a fragment that lacks the first one, two, three, four, five, six, or more nucleotides of SEQ ID NO: 19).
166. The polynucleotide of any one of embodiments 139-156, wherein the 3′ UTR comprises the sequence of SEQ ID NO: 21, or a sequence with at least 80%, 85%, 90%, 95%, 96%, 97%, 98%, 99% or 100% identity to SEQ ID NO: 20, or a fragment thereof (e.g., a fragment that lacks the first one, two, three, four, five, six, or more nucleotides of SEQ ID NO: 20).
167. The polynucleotide of any one of embodiments 139-156, wherein the 3′ UTR comprises the sequence of SEQ ID NO: 21, or a sequence with at least 80%, 85%, 90%, 95%, 96%, 97%, 98%, 99% or 100% identity to SEQ ID NO: 21, or a fragment thereof (e.g., a fragment that lacks the first one, two, three, four, five, six, or more nucleotides of SEQ ID NO: 21).
168. The polynucleotide of any one of embodiments 139-156, wherein the 3′ UTR comprises the sequence of SEQ ID NO: 22, or a sequence with at least 80%, 85%, 90%, 95%, 96%, 97%, 98%, 99% or 100% identity to SEQ ID NO: 22, or a fragment thereof (e.g., a fragment that lacks the first one, two, three, four, five, six, or more nucleotides of SEQ ID NO: 22).
169. The polynucleotide of any one of embodiments 139-156, wherein the 3′ UTR comprises the sequence of SEQ ID NO: 23, or a sequence with at least 80%, 85%, 90%, 95%, 96%, 97%, 98%, 99% or 100% identity to SEQ ID NO: 23, or a fragment thereof (e.g., a fragment that lacks the first one, two, three, four, five, six, or more nucleotides of SEQ ID NO: 23).
170. The polynucleotide of any one of embodiments 139-156, wherein the 3′ UTR comprises the sequence of SEQ ID NO: 24, or a sequence with at least 80%, 85%, 90%, 95%, 96%, 97%, 98%, 99% or 100% identity to SEQ ID NO: 24, or a fragment thereof (e.g., a fragment that lacks the first one, two, three, four, five, six, or more nucleotides of SEQ ID NO: 24).
171. The polynucleotide of any one of embodiments 139-156, wherein the 3′ UTR comprises the sequence of SEQ ID NO: 25, or a sequence with at least 80%, 85%, 90%, 95%, 96%, 97%, 98%, 99% or 100% identity to SEQ ID NO: 25, or a fragment thereof (e.g., a fragment that lacks the first one, two, three, four, five, six, or more nucleotides of SEQ ID NO: 25).
172. The polynucleotide of any one of embodiments 139-156, wherein the 3′ UTR comprises the sequence of SEQ ID NO: 45, or a sequence with at least 80%, 85%, 90%, 95%, 96%, 97%, 98%, 99% or 100% identity to SEQ ID NO: 45, or a fragment thereof (e.g., a fragment that lacks the first one, two, three, four, five, six, or more nucleotides of SEQ ID NO: 45).
173. The polynucleotide of any one of embodiments 139-156, wherein the 3′ UTR comprises the sequence of SEQ ID NO: 79, or a sequence with at least 80%, 85%, 90%, 95%, 96%, 97%, 98%, 99% or 100% identity to SEQ ID NO: 79, or a fragment thereof (e.g., a fragment that lacks the first one, two, three, four, five, six, or more nucleotides of SEQ ID NO: 79).
174. The polynucleotide of any one of embodiments 139-156, wherein the 3′ UTR comprises the sequence of SEQ ID NO: 80, or a sequence with at least 80%, 85%, 90%, 95%, 96%, 97%, 98%, 99% or 100% identity to SEQ ID NO: 80, or a fragment thereof (e.g., a fragment that lacks the first one, two, three, four, five, six, or more nucleotides of SEQ ID NO: 80).
175. The polynucleotide of any one of embodiments 139-156, wherein the 3′ UTR comprises the sequence of SEQ ID NO: 81, or a sequence with at least 80%, 85%, 90%, 95%, 96%, 97%, 98%, 99% or 100% identity to SEQ ID NO: 81, or a fragment thereof (e.g., a fragment that lacks the first one, two, three, four, five, six, or more nucleotides of SEQ ID NO: 81).
176. The polynucleotide of any one of embodiments 139-156, wherein the 3′ UTR comprises the sequence of SEQ ID NO: 82, or a sequence with at least 80%, 85%, 90%, 95%, 96%, 97%, 98%, 99% or 100% identity to SEQ ID NO: 82, or a fragment thereof (e.g., a fragment that lacks the first one, two, three, four, five, six, or more nucleotides of SEQ ID NO: 82).
177. The polynucleotide of any one of embodiments 139-156, wherein the 3′ UTR comprises the sequence of SEQ ID NO: 83, or a sequence with at least 80%, 85%, 90%, 95%, 96%, 97%, 98%, 99% or 100% identity to SEQ ID NO: 83, or a fragment thereof (e.g., a fragment that lacks the first one, two, three, four, five, six, or more nucleotides of SEQ ID NO: 83).
178. The polynucleotide of any one of embodiments 139-156, wherein the 3′ UTR comprises the sequence of SEQ ID NO: 84, or a sequence with at least 80%, 85%, 90%, 95%, 96%, 97%, 98%, 99% or 100% identity to SEQ ID NO: 84, or a fragment thereof (e.g., a fragment that lacks the first one, two, three, four, five, six, or more nucleotides of SEQ ID NO: 84).
179. The polynucleotide of any one of embodiments 139-156, wherein the 3′ UTR comprises the sequence of SEQ ID NO: 85, or a sequence with at least 80%, 85%, 90%, 95%, 96%, 97%, 98%, 99% or 100% identity to SEQ ID NO: 85, or a fragment thereof (e.g., a fragment that lacks the first one, two, three, four, five, six, or more nucleotides of SEQ ID NO: 85).
180. The polynucleotide of any one of embodiments 139-156, wherein the 3′ UTR comprises the sequence of SEQ ID NO: 86, or a sequence with at least 80%, 85%, 90%, 95%, 96%, 97%, 98%, 99% or 100% identity to SEQ ID NO: 86, or a fragment thereof (e.g., a fragment that lacks the first one, two, three, four, five, six, or more nucleotides of SEQ ID NO: 86).
181. The polynucleotide of any one of embodiments 139-156, wherein the 3′ UTR comprises the sequence of SEQ ID NO: 87, or a sequence with at least 80%, 85%, 90%, 95%, 96%, 97%, 98%, 99% or 100% identity to SEQ ID NO: 87, or a fragment thereof (e.g., a fragment that lacks the first one, two, three, four, five, six, or more nucleotides of SEQ ID NO: 87).
182. The polynucleotide of any one of embodiments 139-156, wherein the 3′ UTR comprises the sequence of SEQ ID NO: 94, or a sequence with at least 80%, 85%, 90%, 95%, 96%, 97%, 98%, 99% or 100% identity to SEQ ID NO: 94, or a fragment thereof (e.g., a fragment that lacks the first one, two, three, four, five, six, or more nucleotides of SEQ ID NO: 94).
183. The polynucleotide of any one of embodiments 139-156, wherein the 3′ UTR comprises the sequence of SEQ ID NO: 95, or a sequence with at least 80%, 85%, 90%, 95%, 96%, 97%, 98%, 99% or 100% identity to SEQ ID NO: 95, or a fragment thereof (e.g., a fragment that lacks the first one, two, three, four, five, six, or more nucleotides of SEQ ID NO: 95).
184. The polynucleotide of any one of embodiments 139-156, wherein the 3′ UTR comprises a micro RNA binding site, e.g., as described herein, and/or a TENT recruiting sequence, e.g., as described herein.
185. The polynucleotide of embodiment 184, wherein the miRNA binding site comprises a sequence of any one of SEQ ID NOs: 38-40, and/or wherein the TENT recruiting sequence comprises a sequence of SEQ ID NO: 91 or 92.
186. A polynucleotide encoding a polypeptide, wherein the polynucleotide comprises:

(a) a 5′-UTR (e.g., as described herein);

(b) a coding region comprising a stop element chosen from a stop element provided in Table 3; and

(c) a 3′-UTR (e.g., as described herein).

187. The polynucleotide of embodiment 186, wherein the stop element comprises the sequence of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO:36, SEQ ID NO; 62, SEQ ID NO: 93 or SEQ ID NO: 96.
188. The polynucleotide of embodiment 186, wherein the stop element comprises the sequence of SEQ ID NO: 26.
189. The polynucleotide of embodiment 186, wherein the stop element comprises the sequence of SEQ ID NO: 27.
190. The polynucleotide of embodiment 186, wherein the stop element comprises the sequence of SEQ ID NO: 28.
191. The polynucleotide of embodiment 186, wherein the stop element comprises the sequence of SEQ ID NO: 29.
192. The polynucleotide of embodiment 186, wherein the stop element comprises the sequence of SEQ ID NO: 30.
193. The polynucleotide of embodiment 186, wherein the stop element comprises the sequence of SEQ ID NO: 31.
194. The polynucleotide of embodiment 186, wherein the stop element comprises the sequence of SEQ ID NO: 32.
195. The polynucleotide of embodiment 186, wherein the stop element comprises the sequence of SEQ ID NO: 33.
196. The polynucleotide of embodiment 186, wherein the stop element comprises the sequence of SEQ ID NO: 34.
197. The polynucleotide of embodiment 186, wherein the stop element comprises the sequence of SEQ ID NO: 35.
198. The polynucleotide of embodiment 186, wherein the stop element comprises the sequence of SEQ ID NO: 36.
199. The polynucleotide of embodiment 186, wherein the stop element comprises the sequence of SEQ ID NO: 62.
200. The polynucleotide of embodiment 186, wherein the stop element comprises the sequence of SEQ ID NO: 93.
201. The polynucleotide of embodiment 186, wherein the stop element comprises the sequence of SEQ ID NO: 96.
202. The polynucleotide of embodiment 186, wherein the coding region of (b) comprises a stop element comprising a consensus sequence of Formula B:

(SEQ ID NO: 37)
X₋₃-X₋₂-X₋₁-U-A-A-X₁-X₂-X₃-X₄-X₅-X₆-X₇-X₈-X₉-X₁₀-X₁₁-X₁₂

wherein:

X₁ is a G or A;

X₂, X₄, X₅ X₆ or X₇ is each independently C or U;

X₃ is C or A;

X₈, X₁₀, X₁₁, X₁₂ X₋₁ or X₋₃ is each independently C or G;

X₉ is G or U; and/or

X₋₂ is A or U.

203. The polynucleotide of embodiment 186, wherein the coding region of (b) comprises a stop element comprising a consensus sequence of Formula C

(SEQ ID NO: 56)
X₋₃-X₋₂-X₋₁-U-G-A-X₁-X₂-X₃-X₄-X₅-X₆-X₇-X₈-X₉-X₁₀-X₁₁-X₁₂

wherein:

X₋₃, X₋₁, X₂, X₅, X₆, X₇, X₈, X₉, or X₁₂ is each independently G or C;

X₋₂, X₃, or X₄ is each independent A or C;

X₁ is A or G; and/or

X₁₀ or X₁₁ is each independently C or U.

204. The polynucleotide of embodiment 186, wherein the coding region of (b) comprises a stop element comprising a consensus sequence of Formula D

(SEQ ID NO: 57)
X₋₃-X₋₂-X₋₁-U-A-G-X₁-X₂-X₃-X₄-X₅-X₆-X₇-X₈-X₉-X₁₀-X₁₁-X₁₂

wherein:

X₋₃, X₋₁, X₂, X₃, X₁₀ is each independently G or C;

X₋₂ or X₉ is each independently A or U;

X₁ or X₄ is each independently A or G;

X₅ or X₈ is each independently A or C; and/or

X₆, X₇, X₁₁ or X₁₂ is each independently C or U.

205. The polynucleotide of any one of embodiments 202-204, wherein the consensus sequence has a high GC content, e.g., GC content of about 50%, 60%, 70%, 80%, 90% or 99%.
206. The polynucleotide of any one of embodiments 202-205, which results in an increased half-life of the polynucleotide or an increased level and/or activity, e.g., output, of the polypeptide encoded by the polynucleotide.
207. The polynucleotide of any one of embodiments 202-206, wherein the polynucleotide has about a 1.2-10-fold increase in half-life.
208. The polynucleotide of embodiment 207, wherein the increase in half life is measured using an assay that measures the half-life of a polynucleotide, e.g., an assay described in any one of Examples disclosed herein.
209. The polynucleotide of embodiment 207, wherein the increase in half life is compared to an otherwise similar polynucleotide comprising a coding region which does not have a stop element of embodiment 199.
210. The polynucleotide of embodiment 202, wherein position X₋₁ is the third position of a codon, e.g., a codon specifying an amino acid.
211. The polynucleotide of embodiment 210, wherein the nucleotide at position X₋₁ does not alter the amino acid incorporated into a polypeptide.
212. The polynucleotide of embodiment 202, wherein a C at position X₋₁ results in an increased half-life of the polynucleotide or an increased level and/or activity, e.g., output, of the polypeptide encoded by the polynucleotide.
213. The polynucleotide of embodiment 212, wherein the increased half-life of the polynucleotide is measured by an assay which measures the half-life of a polynucleotide, e.g., an assay described in any one of Examples disclosed herein.
214. The polynucleotide of any one of embodiments 186-213, wherein the stop element results in an increased half-life of the polynucleotide, e.g., about 1.5-20-fold increase in half-life of the polynucleotide.
215. The polynucleotide of embodiment 214, wherein the increase in half-life of the polynucleotide is compared to an otherwise similar polynucleotide which does not have a stop element, has a different stop element, or does not have the stop element of embodiment 186.
216. The polynucleotide of embodiment 214 or 215, wherein the increase in half-life of the polynucleotide is measured according to an assay which measures the half-life of a polynucleotide, e.g., an assay described in any one of Examples disclosed herein.
217. The polynucleotide of any one of embodiments 186-216, wherein the stop element results in an increased level and/or activity, e.g., output or duration of expression, of the polypeptide encoded by the polynucleotide.
218. The polynucleotide of embodiment 217, wherein the increase in level and/or activity, e.g., output or duration of expression, of the polypeptide is measured according to an assay which measures the level and/or activity, e.g., output or duration of expression of a polypeptide, e.g., an assay described in any one of Examples disclosed herein.
219. The polynucleotide of embodiment 218, wherein the stop element results in about 1.5-20-fold increase in level and/or activity, e.g., output, of the polypeptide encoded by the polynucleotide.
220. The polynucleotide of embodiment 218, wherein the stop element results in about 1.5-20 fold increase in level and/or activity, e.g., detectable level or activity, of the polypeptide encoded by the polynucleotide for about 1, 2, 3, 4, 5, 6, 7, 8, 9, 10, or 14 days.
221. The polynucleotide of embodiment 220, wherein the stop element results in detectable level or activity of the polypeptide encoded by the polynucleotide for about 1, 2, 3, 4, 5, 6, 7, 8, 9, 10, or 14 days.
222. The polynucleotide of any one of embodiments 217-221, wherein the increase in level and/or activity, e.g., output or duration of expression, of the polypeptide encoded by the polynucleotide is compared to an otherwise similar polynucleotide which does not have a stop element, has a different stop element, or does not have the stop element of embodiment 186.
223. The polynucleotide of any one of embodiments 186-222, wherein the 5′ UTR of (a) comprises a 5′ UTR sequence provided in Table 1 or a sequence with at least 80%, 85%, 90%, 95%, 96%, 97%, 98%, 99% or 100% identity to a 5′ UTR sequence provided in Table 1, or a variant or a fragment thereof (e.g., a fragment that lacks the first one, two, three, four, five, or six nucleotides of the 5′ UTR sequence provided in Table 1).
224. The polynucleotide of embodiment 223, wherein the 5′ UTR comprises a sequence with at least 80%, 85%, 90%, 95%, 96%, 97%, 98%, 99% or 100% identity to SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 41, SEQ ID NO: 42, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 88, SEQ ID NO: 89 or SEQ ID NO: 90, or a fragment thereof (e.g., a fragment that lacks the first one, two, three, four, five, or six nucleotides of any of the aforesaid sequences).

225. The polynucleotide of embodiment 223, wherein the 5′ UTR comprises a sequence with at least 80%, 85%, 90%, 95%, 96%, 97%, 98%, 99% or 100% identity to SEQ ID NO: 1, or a fragment thereof (e.g., a fragment that lacks the first one, two, three, four, five, six, or more nucleotides of SEQ ID NO: 1).

226. The polynucleotide of embodiment 223, wherein the 5′ UTR comprises a sequence with at least 80%, 85%, 90%, 95%, 96%, 97%, 98%, 99% or 100% identity to SEQ ID NO: 2, or a fragment thereof (e.g., a fragment that lacks the first one, two, three, four, five, six, or more nucleotides of SEQ ID NO: 2).

227. The polynucleotide of embodiment 223, wherein the 5′ UTR comprises a sequence with at least 80%, 85%, 90%, 95%, 96%, 97%, 98%, 99% or 100% identity to SEQ ID NO: 3, or a fragment thereof (e.g., a fragment that lacks the first one, two, three, four, five, six, or more nucleotides of SEQ ID NO: 3).
228. The polynucleotide of embodiment 223, wherein the 5′ UTR comprises a sequence with at least 80%, 85%, 90%, 95%, 96%, 97%, 98%, 99% or 100% identity to SEQ ID NO: 4, or a fragment thereof (e.g., a fragment that lacks the first one, two, three, four, five, six, or more nucleotides of SEQ ID NO: 4).
229. The polynucleotide of embodiment 223, wherein the 5′ UTR comprises a sequence with at least 80%, 85%, 90%, 95%, 96%, 97%, 98%, 99% or 100% identity to SEQ ID NO: 5, or a fragment thereof (e.g., a fragment that lacks the first one, two, three, four, five, six, or more nucleotides of SEQ ID NO: 5).
230. The polynucleotide of embodiment 223, wherein the 5′ UTR comprises a sequence with at least 80%, 85%, 90%, 95%, 96%, 97%, 98%, 99% or 100% identity to SEQ ID NO: 6, or a fragment thereof (e.g., a fragment that lacks the first one, two, three, four, five, six, or more nucleotides of SEQ ID NO: 6).
231. The polynucleotide of embodiment 223, wherein the 5′ UTR comprises a sequence with at least 80%, 85%, 90%, 95%, 96%, 97%, 98%, 99% or 100% identity to SEQ ID NO: 8, or a fragment thereof (e.g., a fragment that lacks the first one, two, three, four, five, six, or more nucleotides of SEQ ID NO: 8).

232. The polynucleotide of embodiment 223, wherein the 5′ UTR comprises a sequence with at least 80%, 85%, 90%, 95%, 96%, 97%, 98%, 99% or 100% identity to SEQ ID NO: 41, or a fragment thereof (e.g., a fragment that lacks the first one, two, three, four, five, six, or more nucleotides of SEQ ID NO: 41).

233. The polynucleotide of embodiment 223, wherein the 5′ UTR comprises a sequence with at least 80%, 85%, 90%, 95%, 96%, 97%, 98%, 99% or 100% identity to SEQ ID NO: 42, or a fragment thereof (e.g., a fragment that lacks the first one, two, three, four, five, six, or more nucleotides of SEQ ID NO: 42).
234. The polynucleotide of embodiment 223, wherein the 5′ UTR comprises the sequence of SEQ ID NO: 63 or a fragment thereof (e.g., a fragment that lacks the first one, two, three, four, five, or six nucleotides of SEQ ID NO: 63).
235. The polynucleotide of embodiment 223, wherein the 5′ UTR comprises the sequence of SEQ ID NO: 64 or a fragment thereof (e.g., a fragment that lacks the first one, two, three, four, five, or six nucleotides of SEQ ID NO: 64).
236. The polynucleotide of embodiment 223, wherein the 5′ UTR comprises the sequence of SEQ ID NO: 65 or a fragment thereof (e.g., a fragment that lacks the first one, two, three, four, five, or six nucleotides of SEQ ID NO: 65).
237. The polynucleotide of embodiment 223, wherein the 5′ UTR comprises the sequence of SEQ ID NO: 66 or a fragment thereof (e.g., a fragment that lacks the first one, two, three, four, five, or six nucleotides of SEQ ID NO: 66).
238. The polynucleotide of embodiment 223, wherein the 5′ UTR comprises the sequence of SEQ ID NO: 67 or a fragment thereof (e.g., a fragment that lacks the first one, two, three, four, five, or six nucleotides of SEQ ID NO: 67).
239. The polynucleotide of embodiment 223, wherein the 5′ UTR comprises the sequence of SEQ ID NO: 68 or a fragment thereof (e.g., a fragment that lacks the first one, two, three, four, five, or six nucleotides of SEQ ID NO: 68).
240. The polynucleotide of embodiment 223, wherein the 5′ UTR comprises the sequence of SEQ ID NO: 69 or a fragment thereof (e.g., a fragment that lacks the first one, two, three, four, five, or six nucleotides of SEQ ID NO: 69).
241. The polynucleotide of embodiment 223, wherein the 5′ UTR comprises the sequence of SEQ ID NO: 70 or a fragment thereof (e.g., a fragment that lacks the first one, two, three, four, five, or six nucleotides of SEQ ID NO: 70).
242. The polynucleotide of embodiment 223, wherein the 5′ UTR comprises the sequence of SEQ ID NO: 70 or a fragment thereof (e.g., a fragment that lacks the first one, two, three, four, five, or six nucleotides of SEQ ID NO: 70).
243. The polynucleotide of embodiment 223, wherein the 5′ UTR comprises the sequence of SEQ ID NO: 71 or a fragment thereof (e.g., a fragment that lacks the first one, two, three, four, five, or six nucleotides of SEQ ID NO: 71).
244. The polynucleotide of embodiment 223, wherein the 5′ UTR comprises the sequence of SEQ ID NO: 72 or a fragment thereof (e.g., a fragment that lacks the first one, two, three, four, five, or six nucleotides of SEQ ID NO: 72).
245. The polynucleotide of embodiment 223, wherein the 5′ UTR comprises the sequence of SEQ ID NO: 73 or a fragment thereof (e.g., a fragment that lacks the first one, two, three, four, five, or six nucleotides of SEQ ID NO: 73).
246. The polynucleotide of embodiment 223, wherein the 5′ UTR comprises the sequence of SEQ ID NO: 74 or a fragment thereof (e.g., a fragment that lacks the first one, two, three, four, five, or six nucleotides of SEQ ID NO: 74).
247. The polynucleotide of embodiment 223, wherein the 5′ UTR comprises the sequence of SEQ ID NO: 75 or a fragment thereof (e.g., a fragment that lacks the first one, two, three, four, five, or six nucleotides of SEQ ID NO: 75).
248. The polynucleotide of embodiment 223, wherein the 5′ UTR comprises the sequence of SEQ ID NO: 76 or a fragment thereof (e.g., a fragment that lacks the first one, two, three, four, five, or six nucleotides of SEQ ID NO: 76).
249. The polynucleotide of embodiment 223, wherein the 5′ UTR comprises the sequence of SEQ ID NO: 77 or a fragment thereof (e.g., a fragment that lacks the first one, two, three, four, five, or six nucleotides of SEQ ID NO: 77).
250. The polynucleotide of embodiment 223, wherein the 5′ UTR comprises the sequence of SEQ ID NO: 78 or a fragment thereof (e.g., a fragment that lacks the first one, two, three, four, five, or six nucleotides of SEQ ID NO: 78).
251. The polynucleotide of embodiment 223, wherein the 5′ UTR comprises the sequence of SEQ ID NO: 88 or a fragment thereof (e.g., a fragment that lacks the first one, two, three, four, five, or six nucleotides of SEQ ID NO: 88).
252. The polynucleotide of embodiment 223, wherein the 5′ UTR comprises the sequence of SEQ ID NO: 89 or a fragment thereof (e.g., a fragment that lacks the first one, two, three, four, five, or six nucleotides of SEQ ID NO: 89).
253. The polynucleotide of embodiment 223, wherein the 5′ UTR comprises the sequence of SEQ ID NO: 90 or a fragment thereof (e.g., a fragment that lacks the first one, two, three, four, five, or six nucleotides of SEQ ID NO: 90).
254. The polynucleotide of any one of embodiments 186-222, wherein the 3′ UTR of (c) comprises a 3′ UTR sequence provided in Table 2 or a sequence with at least 80%, 85%, 90%, 95%, 96%, 97%, 98%, 99% or 100% identity to a 3′ UTR sequence provided in Table 2, or a fragment thereof (e.g., a fragment that lacks the first one, two, three, four, five, six, or more nucleotides of the 3′ UTR sequence provided in Table 2)

optionally wherein the polynucleotide comprises a sequence with at least 80%, 85%, 90%, 95%, 96%, 97%, 98%, 99% or 100% identity to a sequence provided in Table 4.

255. The polynucleotide of embodiment 254, wherein the 3′ UTR comprises a sequence with at least 80%, 85%, 90%, 95%, 96%, 97%, 98%, 99% or 100% identity to SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 45, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 94 or SEQ ID NO: 95, or a fragment thereof (e.g., a fragment that lacks the first one, two, three, four, five, six, or more nucleotides of any of the aforesaid sequences)

optionally wherein the polynucleotide comprises a sequence with at least 80%, 85%, 90%, 95%, 96%, 97%, 98%, 99% or 100% identity to any of SEQ ID NOs: 47, 48, 49, 50, 122, 52, 53, 54, 55, 59, 60, 61, 126, 127, 97, 98, 99, 100, 101, 102, 103, 104, 105, 106, 107, 108, 109, 110, 111, 112, 113, 114, 115, 116, 117, 118, 119, or 120, or a variant or fragment thereof.

256. The polynucleotide of embodiment 254, wherein the 3′ UTR comprises the sequence of SEQ ID NO: 11, or a sequence with at least 80%, 85%, 90%, 95%, 96%, 97%, 98%, 99% or 100% identity to SEQ ID NO: 11, or a fragment thereof (e.g., a fragment that lacks the first one, two, three, four, five, six, or more nucleotides of SEQ ID NO: 11).
257. The polynucleotide of embodiment 254, wherein the 3′ UTR comprises the sequence of SEQ ID NO: 12, or a sequence with at least 80%, 85%, 90%, 95%, 96%, 97%, 98%, 99% or 100% identity to SEQ ID NO: 12, or a fragment thereof (e.g., a fragment that lacks the first one, two, three, four, five, six, or more nucleotides of SEQ ID NO: 12).
258. The polynucleotide of embodiment 254, wherein the 3′ UTR comprises the sequence of SEQ ID NO: 13, or a sequence with at least 80%, 85%, 90%, 95%, 96%, 97%, 98%, 99% or 100% identity to SEQ ID NO: 13, or a fragment thereof (e.g., a fragment that lacks the first one, two, three, four, five, six, or more nucleotides of SEQ ID NO: 13).
259. The polynucleotide of embodiment 254, wherein the 3′ UTR comprises the sequence of SEQ ID NO: 14, or a sequence with at least 80%, 85%, 90%, 95%, 96%, 97%, 98%, 99% or 100% identity to SEQ ID NO: 14, or a fragment thereof (e.g., a fragment that lacks the first one, two, three, four, five, six, or more nucleotides of SEQ ID NO: 14).
260. The polynucleotide of embodiment 254, wherein the 3′ UTR comprises the sequence of SEQ ID NO: 15, or a sequence with at least 80%, 85%, 90%, 95%, 96%, 97%, 98%, 99% or 100% identity to SEQ ID NO: 15, or a fragment thereof (e.g., a fragment that lacks the first one, two, three, four, five, six, or more nucleotides of SEQ ID NO: 15).
261. The polynucleotide of embodiment 254, wherein the 3′ UTR comprises the sequence of SEQ ID NO: 16, or a sequence with at least 80%, 85%, 90%, 95%, 96%, 97%, 98%, 99% or 100% identity to SEQ ID NO: 16, or a fragment thereof (e.g., a fragment that lacks the first one, two, three, four, five, six, or more nucleotides of SEQ ID NO: 16).
262. The polynucleotide of embodiment 254, wherein the 3′ UTR comprises the sequence of SEQ ID NO: 17, or a sequence with at least 80%, 85%, 90%, 95%, 96%, 97%, 98%, 99% or 100% identity to SEQ ID NO: 17, or a fragment thereof (e.g., a fragment that lacks the first one, two, three, four, five, six, or more nucleotides of SEQ ID NO: 17).
263. The polynucleotide of embodiment 254, wherein the 3′ UTR comprises the sequence of SEQ ID NO: 18, or a sequence with at least 80%, 85%, 90%, 95%, 96%, 97%, 98%, 99% or 100% identity to SEQ ID NO: 18, or a fragment thereof (e.g., a fragment that lacks the first one, two, three, four, five, six, or more nucleotides of SEQ ID NO: 18).
264. The polynucleotide of embodiment 254, wherein the 3′ UTR comprises the sequence of SEQ ID NO: 19, or a sequence with at least 80%, 85%, 90%, 95%, 96%, 97%, 98%, 99% or 100% identity to SEQ ID NO: 19, or a fragment thereof (e.g., a fragment that lacks the first one, two, three, four, five, six, or more nucleotides of SEQ ID NO: 19).
265. The polynucleotide of embodiment 254, wherein the 3′ UTR comprises the sequence of SEQ ID NO: 21, or a sequence with at least 80%, 85%, 90%, 95%, 96%, 97%, 98%, 99% or 100% identity to SEQ ID NO: 20 or a fragment thereof (e.g., a fragment that lacks the first one, two, three, four, five, six, or more nucleotides of any of the aforesaid sequences).
266. The polynucleotide of embodiment 254, wherein the 3′ UTR comprises the sequence of SEQ ID NO: 21, or a sequence with at least 80%, 85%, 90%, 95%, 96%, 97%, 98%, 99% or 100% identity to SEQ ID NO: 21, or a fragment thereof (e.g., a fragment that lacks the first one, two, three, four, five, six, or more nucleotides of SEQ ID NO: 21).
267. The polynucleotide of embodiment 254, wherein the 3′ UTR comprises the sequence of SEQ ID NO: 22, or a sequence with at least 80%, 85%, 90%, 95%, 96%, 97%, 98%, 99% or 100% identity to SEQ ID NO: 22, or a fragment thereof (e.g., a fragment that lacks the first one, two, three, four, five, six, or more nucleotides of SEQ ID NO: 22).
268. The polynucleotide of embodiment 254, wherein the 3′ UTR comprises the sequence of SEQ ID NO: 23, or a sequence with at least 80%, 85%, 90%, 95%, 96%, 97%, 98%, 99% or 100% identity to SEQ ID NO: 23, or a fragment thereof (e.g., a fragment that lacks the first one, two, three, four, five, six, or more nucleotides of SEQ ID NO: 23).
269. The polynucleotide of embodiment 254, wherein the 3′ UTR comprises the sequence of SEQ ID NO: 24, or a sequence with at least 80%, 85%, 90%, 95%, 96%, 97%, 98%, 99% or 100% identity to SEQ ID NO: 24, or a fragment thereof (e.g., a fragment that lacks the first one, two, three, four, five, six, or more nucleotides of SEQ ID NO: 24).
270. The polynucleotide of embodiment 254, wherein the 3′ UTR comprises the sequence of SEQ ID NO: 25, or a sequence with at least 80%, 85%, 90%, 95%, 96%, 97%, 98%, 99% or 100% identity to SEQ ID NO: 25, or a fragment thereof (e.g., a fragment that lacks the first one, two, three, four, five, six, or more nucleotides of SEQ ID NO: 25).
271. The polynucleotide of claim 254, wherein the 3′ UTR comprises the sequence of SEQ ID NO: 45, or a sequence with at least 80%, 85%, 90%, 95%, 96%, 97%, 98%, 99% or 100% identity to SEQ ID NO: 45, or a fragment thereof (e.g., a fragment that lacks the first one, two, three, four, five, six, or more nucleotides of SEQ ID NO: 45).
272. The polynucleotide of claim 254, wherein the 3′ UTR comprises the sequence of SEQ ID NO: 79, or a sequence with at least 80%, 85%, 90%, 95%, 96%, 97%, 98%, 99% or 100% identity to SEQ ID NO: 79, or a fragment thereof (e.g., a fragment that lacks the first one, two, three, four, five, six, or more nucleotides of SEQ ID NO: 79).
273. The polynucleotide of claim 254, wherein the 3′ UTR comprises the sequence of SEQ ID NO: 80, or a sequence with at least 80%, 85%, 90%, 95%, 96%, 97%, 98%, 99% or 100% identity to SEQ ID NO: 80, or a fragment thereof (e.g., a fragment that lacks the first one, two, three, four, five, six, or more nucleotides of SEQ ID NO: 80).
274. The polynucleotide of claim 254, wherein the 3′ UTR comprises the sequence of SEQ ID NO: 81, or a sequence with at least 80%, 85%, 90%, 95%, 96%, 97%, 98%, 99% or 100% identity to SEQ ID NO: 81, or a fragment thereof (e.g., a fragment that lacks the first one, two, three, four, five, six, or more nucleotides of SEQ ID NO: 81).
275. The polynucleotide of claim 254, wherein the 3′ UTR comprises the sequence of SEQ ID NO: 82, or a sequence with at least 80%, 85%, 90%, 95%, 96%, 97%, 98%, 99% or 100% identity to SEQ ID NO: 82, or a fragment thereof (e.g., a fragment that lacks the first one, two, three, four, five, six, or more nucleotides of SEQ ID NO: 82).
276. The polynucleotide of claim 254, wherein the 3′ UTR comprises the sequence of SEQ ID NO: 83, or a sequence with at least 80%, 85%, 90%, 95%, 96%, 97%, 98%, 99% or 100% identity to SEQ ID NO: 83, or a fragment thereof (e.g., a fragment that lacks the first one, two, three, four, five, six, or more nucleotides of SEQ ID NO: 83).
277. The polynucleotide of claim 254, wherein the 3′ UTR comprises the sequence of SEQ ID NO: 84, or a sequence with at least 80%, 85%, 90%, 95%, 96%, 97%, 98%, 99% or 100% identity to SEQ ID NO: 84, or a fragment thereof (e.g., a fragment that lacks the first one, two, three, four, five, six, or more nucleotides of SEQ ID NO: 84).
278. The polynucleotide of claim 254, wherein the 3′ UTR comprises the sequence of SEQ ID NO: 85, or a sequence with at least 80%, 85%, 90%, 95%, 96%, 97%, 98%, 99% or 100% identity to SEQ ID NO: 85, or a fragment thereof (e.g., a fragment that lacks the first one, two, three, four, five, six, or more nucleotides of SEQ ID NO: 85).
279. The polynucleotide of claim 254, wherein the 3′ UTR comprises the sequence of SEQ ID NO: 86, or a sequence with at least 80%, 85%, 90%, 95%, 96%, 97%, 98%, 99% or 100% identity to SEQ ID NO: 86, or a fragment thereof (e.g., a fragment that lacks the first one, two, three, four, five, six, or more nucleotides of SEQ ID NO: 86).
280. The polynucleotide of claim 254, wherein the 3′ UTR comprises the sequence of SEQ ID NO: 87, or a sequence with at least 80%, 85%, 90%, 95%, 96%, 97%, 98%, 99% or 100% identity to SEQ ID NO: 87, or a fragment thereof (e.g., a fragment that lacks the first one, two, three, four, five, six, or more nucleotides of SEQ ID NO: 87).
281. The polynucleotide of claim 254, wherein the 3′ UTR comprises the sequence of SEQ ID NO: 94, or a sequence with at least 80%, 85%, 90%, 95%, 96%, 97%, 98%, 99% or 100% identity to SEQ ID NO: 94, or a fragment thereof (e.g., a fragment that lacks the first one, two, three, four, five, six, or more nucleotides of SEQ ID NO: 94).
282. The polynucleotide of claim 254, wherein the 3′ UTR comprises the sequence of SEQ ID NO: 95, or a sequence with at least 80%, 85%, 90%, 95%, 96%, 97%, 98%, 99% or 100% identity to SEQ ID NO: 95, or a fragment thereof (e.g., a fragment that lacks the first one, two, three, four, five, six, or more nucleotides of SEQ ID NO: 95).
283. The polynucleotide of embodiment 254, wherein the 3′ UTR comprises a micro RNA binding site, e.g., as described herein, and/or a TENT recruiting sequence, e.g., as described herein.
284. The polynucleotide of embodiment 254, wherein the miRNA binding site comprises a sequence of any one of SEQ ID NOs: 38-40, or wherein the TENT recruiting sequence comprises a sequence of SEQ ID NO: 91 or 92.
285. The polynucleotide of any one of embodiments 186-222, wherein:

(i) the 5′ UTR of (a) comprises a 5′ UTR sequence provided in Table 1 or a sequence with at least 80%, 85%, 90%, 95%, 96%, 97%, 98%, 99% or 100% identity to a 3′ UTR sequence provided in Table 1, or a fragment thereof (e.g., a fragment that lacks the first one, two, three, four, five, or six nucleotides of the 5′ UTR sequence provided in Table 1); and

(ii) the 3′ UTR of (c) comprises a 3′ UTR sequence provided in Table 2 or a sequence with at least 80%, 85%, 90%, 95%, 96%, 97%, 98%, 99% or 100% identity to a 3′ UTR sequence provided in Table 2, or a fragment thereof (e.g., a fragment that lacks the first one, two, three, four, five, six, or more nucleotides of the 3′ UTR sequence provided in Table 2)

optionally wherein the polynucleotide comprises a sequence with at least 80%, 85%, 90%, 95%, 96%, 97%, 98%, 99% or 100% identity to a sequence provided in Table 4, e.g., any of SEQ ID NOs: 47, 48, 49, 50, 122, 52, 53, 54, 55, 59, 60, 61, 126, 127, 97, 98, 99, 100, 101, 102, 103, 104, 105, 106, 107, 108, 109, 110, 111, 112, 113, 114, 115, 116, 117, 118, 119, or 120, or a variant or fragment thereof.

286. The polynucleotide of embodiment 285, wherein the 5′ UTR comprises a sequence with at least 80%, 85%, 90%, 95%, 96%, 97%, 98%, 99% or 100% identity to SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 41, SEQ ID NO: 42, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 88, SEQ ID NO: 89 or SEQ ID NO: 90, or a fragment thereof (e.g., a fragment that lacks the first one, two, three, four, five, or six nucleotides of any of the aforesaid sequences).
287. The polynucleotide of embodiment 285, wherein the 5′ UTR comprises a sequence with at least 80%, 85%, 90%, 95%, 96%, 97%, 98%, 99% or 100% identity to SEQ ID NO: 1, or a fragment thereof (e.g., a fragment that lacks the first one, two, three, four, five, six, or more nucleotides of SEQ ID NO: 1).
288. The polynucleotide of embodiment 285, wherein the 5′ UTR comprises a sequence with at least 80%, 85%, 90%, 95%, 96%, 97%, 98%, 99% or 100% identity to SEQ ID NO: 2, or a fragment thereof (e.g., a fragment that lacks the first one, two, three, four, five, six, or more nucleotides of SEQ ID NO: 2).
289. The polynucleotide of embodiment 285, wherein the 5′ UTR comprises a sequence with at least 80%, 85%, 90%, 95%, 96%, 97%, 98%, 99% or 100% identity to SEQ ID NO: 3, or a fragment thereof (e.g., a fragment that lacks the first one, two, three, four, five, six, or more nucleotides of SEQ ID NO: 3).
290. The polynucleotide of embodiment 285, wherein the 5′ UTR comprises a sequence with at least 80%, 85%, 90%, 95%, 96%, 97%, 98%, 99% or 100% identity to SEQ ID NO: 4, or a fragment thereof (e.g., a fragment that lacks the first one, two, three, four, five, six, or more nucleotides of SEQ ID NO: 4).
291. The polynucleotide of embodiment 285, wherein the 5′ UTR comprises a sequence with at least 80%, 85%, 90%, 95%, 96%, 97%, 98%, 99% or 100% identity to SEQ ID NO: 5, or a fragment thereof (e.g., a fragment that lacks the first one, two, three, four, five, six, or more nucleotides of SEQ ID NO: 5).
292. The polynucleotide of embodiment 285, wherein the 5′ UTR comprises a sequence with at least 80%, 85%, 90%, 95%, 96%, 97%, 98%, 99% or 100% identity to SEQ ID NO: 6, or a fragment thereof (e.g., a fragment that lacks the first one, two, three, four, five, six, or more nucleotides of SEQ ID NO: 6).
293. The polynucleotide of embodiment 285, wherein the 5′ UTR comprises a sequence with at least 80%, 85%, 90%, 95%, 96%, 97%, 98%, 99% or 100% identity to SEQ ID NO: 8, or a fragment thereof (e.g., a fragment that lacks the first one, two, three, four, five, six, or more nucleotides of SEQ ID NO: 8).
294. The polynucleotide of embodiment 285, wherein the 5′ UTR comprises a sequence with at least 80%, 85%, 90%, 95%, 96%, 97%, 98%, 99% or 100% identity to SEQ ID NO: 41, or a fragment thereof (e.g., a fragment that lacks the first one, two, three, four, five, six, or more nucleotides of SEQ ID NO: 41).
295. The polynucleotide of embodiment 285, wherein the 5′ UTR comprises a sequence with at least 80%, 85%, 90%, 95%, 96%, 97%, 98%, 99% or 100% identity to SEQ ID NO: 42, or a fragment thereof (e.g., a fragment that lacks the first one, two, three, four, five, six, or more nucleotides of SEQ ID NO: 42).
296. The polynucleotide of embodiment 285, wherein the 5′ UTR comprises the sequence of SEQ ID NO: 63 or a fragment thereof (e.g., a fragment that lacks the first one, two, three, four, five, or six nucleotides of SEQ ID NO: 63).
297. The polynucleotide of embodiment 285, wherein the 5′ UTR comprises the sequence of SEQ ID NO: 64 or a fragment thereof (e.g., a fragment that lacks the first one, two, three, four, five, or six nucleotides of SEQ ID NO: 64).
298. The polynucleotide of embodiment 285, wherein the 5′ UTR comprises the sequence of SEQ ID NO: 65 or a fragment thereof (e.g., a fragment that lacks the first one, two, three, four, five, or six nucleotides of SEQ ID NO: 65).
299. The polynucleotide of embodiment 285, wherein the 5′ UTR comprises the sequence of SEQ ID NO: 66 or a fragment thereof (e.g., a fragment that lacks the first one, two, three, four, five, or six nucleotides of SEQ ID NO: 66).
300. The polynucleotide of embodiment 285, wherein the 5′ UTR comprises the sequence of SEQ ID NO: 67 or a fragment thereof (e.g., a fragment that lacks the first one, two, three, four, five, or six nucleotides of SEQ ID NO: 67).
301. The polynucleotide of embodiment 285, wherein the 5′ UTR comprises the sequence of SEQ ID NO: 68 or a fragment thereof (e.g., a fragment that lacks the first one, two, three, four, five, or six nucleotides of SEQ ID NO: 68).
302. The polynucleotide of embodiment 285, wherein the 5′ UTR comprises the sequence of SEQ ID NO: 69 or a fragment thereof (e.g., a fragment that lacks the first one, two, three, four, five, or six nucleotides of SEQ ID NO: 69).
303. The polynucleotide of embodiment 285, wherein the 5′ UTR comprises the sequence of SEQ ID NO: 70 or a fragment thereof (e.g., a fragment that lacks the first one, two, three, four, five, or six nucleotides of SEQ ID NO: 70).
304. The polynucleotide of embodiment 285, wherein the 5′ UTR comprises the sequence of SEQ ID NO: 70 or a fragment thereof (e.g., a fragment that lacks the first one, two, three, four, five, or six nucleotides of SEQ ID NO: 70).
305. The polynucleotide of embodiment 285, wherein the 5′ UTR comprises the sequence of SEQ ID NO: 71 or a fragment thereof (e.g., a fragment that lacks the first one, two, three, four, five, or six nucleotides of SEQ ID NO: 71).
306. The polynucleotide of embodiment 285, wherein the 5′ UTR comprises the sequence of SEQ ID NO: 72 or a fragment thereof (e.g., a fragment that lacks the first one, two, three, four, five, or six nucleotides of SEQ ID NO: 72).
307. The polynucleotide of embodiment 285, wherein the 5′ UTR comprises the sequence of SEQ ID NO: 73 or a fragment thereof (e.g., a fragment that lacks the first one, two, three, four, five, or six nucleotides of SEQ ID NO: 73).
308. The polynucleotide of embodiment 285, wherein the 5′ UTR comprises the sequence of SEQ ID NO: 74 or a fragment thereof (e.g., a fragment that lacks the first one, two, three, four, five, or six nucleotides of SEQ ID NO: 74).
309. The polynucleotide of embodiment 285, wherein the 5′ UTR comprises the sequence of SEQ ID NO: 75 or a fragment thereof (e.g., a fragment that lacks the first one, two, three, four, five, or six nucleotides of SEQ ID NO: 75).
310. The polynucleotide of embodiment 285, wherein the 5′ UTR comprises the sequence of SEQ ID NO: 76 or a fragment thereof (e.g., a fragment that lacks the first one, two, three, four, five, or six nucleotides of SEQ ID NO: 76).
311. The polynucleotide of embodiment 285, wherein the 5′ UTR comprises the sequence of SEQ ID NO: 77 or a fragment thereof (e.g., a fragment that lacks the first one, two, three, four, five, or six nucleotides of SEQ ID NO: 77).
312. The polynucleotide of embodiment 285, wherein the 5′ UTR comprises the sequence of SEQ ID NO: 78 or a fragment thereof (e.g., a fragment that lacks the first one, two, three, four, five, or six nucleotides of SEQ ID NO: 87).
313. The polynucleotide of embodiment 285, wherein the 5′ UTR comprises the sequence of SEQ ID NO: 88 or a fragment thereof (e.g., a fragment that lacks the first one, two, three, four, five, or six nucleotides of SEQ ID NO: 88).
314. The polynucleotide of embodiment 285, wherein the 5′ UTR comprises the sequence of SEQ ID NO: 89 or a fragment thereof (e.g., a fragment that lacks the first one, two, three, four, five, or six nucleotides of SEQ ID NO: 89).
315. The polynucleotide of embodiment 285, wherein the 5′ UTR comprises the sequence of SEQ ID NO: 90 or a fragment thereof (e.g., a fragment that lacks the first one, two, three, four, five, or six nucleotides of SEQ ID NO: 90).
316. The polynucleotide of any one of embodiments 285-315, wherein the 3′ UTR comprises a sequence with at least 80%, 85%, 90%, 95%, 96%, 97%, 98%, 99% or 100% identity to SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 45, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 94 or SEQ ID NO: 95, or a fragment thereof (e.g., a fragment that lacks the first one, two, three, four, five, six, or more nucleotides of any of the aforesaid sequences).
317. The polynucleotide of embodiment any one of embodiments 285-315, wherein the 3′ UTR comprises the sequence of SEQ ID NO: 11, or a sequence with at least 80%, 85%, 90%, 95%, 96%, 97%, 98%, 99% or 100% identity to SEQ ID NO: 11, or a fragment thereof (e.g., a fragment that lacks the first one, two, three, four, five, six, or more nucleotides of SEQ ID NO: 11).
318. The polynucleotide of embodiment any one of embodiments 285-315, wherein the 3′ UTR comprises the sequence of SEQ ID NO: 12, or a sequence with at least 80%, 85%, 90%, 95%, 96%, 97%, 98%, 99% or 100% identity to SEQ ID NO: 12, or a fragment thereof (e.g., a fragment that lacks the first one, two, three, four, five, six, or more nucleotides of SEQ ID NO: 12).
319. The polynucleotide of embodiment any one of embodiments 285-315, wherein the 3′ UTR comprises the sequence of SEQ ID NO: 13, or a sequence with at least 80%, 85%, 90%, 95%, 96%, 97%, 98%, 99% or 100% identity to SEQ ID NO: 13, or a fragment thereof (e.g., a fragment that lacks the first one, two, three, four, five, six, or more nucleotides of SEQ ID NO: 13).
320. The polynucleotide of embodiment any one of embodiments 285-315, wherein the 3′ UTR comprises the sequence of SEQ ID NO: 14, or a sequence with at least 80%, 85%, 90%, 95%, 96%, 97%, 98%, 99% or 100% identity to SEQ ID NO: 14, or a fragment thereof (e.g., a fragment that lacks the first one, two, three, four, five, six, or more nucleotides of SEQ ID NO: 14).
321. The polynucleotide of embodiment any one of embodiments 285-315, wherein the 3′ UTR comprises the sequence of SEQ ID NO: 15, or a sequence with at least 80%, 85%, 90%, 95%, 96%, 97%, 98%, 99% or 100% identity to SEQ ID NO: 15, or a fragment thereof (e.g., a fragment that lacks the first one, two, three, four, five, six, or more nucleotides of SEQ ID NO: 15).
322. The polynucleotide of embodiment any one of embodiments 285-315, wherein the 3′ UTR comprises the sequence of SEQ ID NO: 16, or a sequence with at least 80%, 85%, 90%, 95%, 96%, 97%, 98%, 99% or 100% identity to SEQ ID NO: 16, or a fragment thereof (e.g., a fragment that lacks the first one, two, three, four, five, six, or more nucleotides of SEQ ID NO: 16).
323. The polynucleotide of embodiment any one of embodiments 285-315, wherein the 3′ UTR comprises the sequence of SEQ ID NO: 17, or a sequence with at least 80%, 85%, 90%, 95%, 96%, 97%, 98%, 99% or 100% identity to SEQ ID NO: 17, or a fragment thereof (e.g., a fragment that lacks the first one, two, three, four, five, six, or more nucleotides of SEQ ID NO: 17).
324. The polynucleotide of embodiment any one of embodiments 285-315, wherein the 3′ UTR comprises the sequence of SEQ ID NO: 18, or a sequence with at least 80%, 85%, 90%, 95%, 96%, 97%, 98%, 99% or 100% identity to SEQ ID NO: 18, or a fragment thereof (e.g., a fragment that lacks the first one, two, three, four, five, six, or more nucleotides of SEQ ID NO: 18).
325. The polynucleotide of embodiment any one of embodiments 285-315, wherein the 3′ UTR comprises the sequence of SEQ ID NO: 19, or a sequence with at least 80%, 85%, 90%, 95%, 96%, 97%, 98%, 99% or 100% identity to SEQ ID NO: 19, or a fragment thereof (e.g., a fragment that lacks the first one, two, three, four, five, six, or more nucleotides of SEQ ID NO: 19).
326. The polynucleotide of embodiment any one of embodiments 285-315, wherein the 3′ UTR comprises the sequence of SEQ ID NO: 20, or a sequence with at least 80%, 85%, 90%, 95%, 96%, 97%, 98%, 99% or 100% identity to SEQ ID NO: 20, or a fragment thereof (e.g., a fragment that lacks the first one, two, three, four, five, six, or more nucleotides of SEQ ID NO: 20).
327. The polynucleotide of embodiment any one of embodiments 285-315, wherein the 3′ UTR comprises the sequence of SEQ ID NO: 21, or a sequence with at least 80%, 85%, 90%, 95%, 96%, 97%, 98%, 99% or 100% identity to SEQ ID NO: 21, or a fragment thereof (e.g., a fragment that lacks the first one, two, three, four, five, six, or more nucleotides of SEQ ID NO: 21).
328. The polynucleotide of embodiment any one of embodiments 285-315, wherein the 3′ UTR comprises the sequence of SEQ ID NO: 22, or a sequence with at least 80%, 85%, 90%, 95%, 96%, 97%, 98%, 99% or 100% identity to SEQ ID NO: 22, or a fragment thereof (e.g., a fragment that lacks the first one, two, three, four, five, six, or more nucleotides of SEQ ID NO: 22).
329. The polynucleotide of embodiment any one of embodiments 285-315, wherein the 3′ UTR comprises the sequence of SEQ ID NO: 23, or a sequence with at least 80%, 85%, 90%, 95%, 96%, 97%, 98%, 99% or 100% identity to SEQ ID NO: 23, or a fragment thereof (e.g., a fragment that lacks the first one, two, three, four, five, six, or more nucleotides of SEQ ID NO: 23).
330. The polynucleotide of embodiment any one of embodiments 285-315, wherein the 3′ UTR comprises the sequence of SEQ ID NO: 24, or a sequence with at least 80%, 85%, 90%, 95%, 96%, 97%, 98%, 99% or 100% identity to SEQ ID NO: 24, or a fragment thereof (e.g., a fragment that lacks the first one, two, three, four, five, six, or more nucleotides of SEQ ID NO: 24).
331. The polynucleotide of embodiment any one of embodiments 285-315, wherein the 3′ UTR comprises the sequence of SEQ ID NO: 25, or a sequence with at least 80%, 85%, 90%, 95%, 96%, 97%, 98%, 99% or 100% identity to SEQ ID NO: 25, or a fragment thereof (e.g., a fragment that lacks the first one, two, three, four, five, six, or more nucleotides of SEQ ID NO: 25).
332. The polynucleotide of any one of embodiments 285-315, wherein the 3′ UTR comprises the sequence of SEQ ID NO: 45, or a sequence with at least 80%, 85%, 90%, 95%, 96%, 97%, 98%, 99% or 100% identity to SEQ ID NO: 45, or a fragment thereof (e.g., a fragment that lacks the first one, two, three, four, five, six, or more nucleotides of SEQ ID NO: 45).
333. The polynucleotide of any one of embodiments 285-315, wherein the 3′ UTR comprises the sequence of SEQ ID NO: 79, or a sequence with at least 80%, 85%, 90%, 95%, 96%, 97%, 98%, 99% or 100% identity to SEQ ID NO: 79, or a fragment thereof (e.g., a fragment that lacks the first one, two, three, four, five, six, or more nucleotides of SEQ ID NO: 79).
334. The polynucleotide of any one of embodiments 285-315, wherein the 3′ UTR comprises the sequence of SEQ ID NO: 80, or a sequence with at least 80%, 85%, 90%, 95%, 96%, 97%, 98%, 99% or 100% identity to SEQ ID NO: 80, or a fragment thereof (e.g., a fragment that lacks the first one, two, three, four, five, six, or more nucleotides of SEQ ID NO: 80).
335. The polynucleotide of any one of embodiments 285-315, wherein the 3′ UTR comprises the sequence of SEQ ID NO: 81, or a sequence with at least 80%, 85%, 90%, 95%, 96%, 97%, 98%, 99% or 100% identity to SEQ ID NO: 81, or a fragment thereof (e.g., a fragment that lacks the first one, two, three, four, five, six, or more nucleotides of SEQ ID NO: 81).
336. The polynucleotide of any one of embodiments 285-315, wherein the 3′ UTR comprises the sequence of SEQ ID NO: 82, or a sequence with at least 80%, 85%, 90%, 95%, 96%, 97%, 98%, 99% or 100% identity to SEQ ID NO: 82, or a fragment thereof (e.g., a fragment that lacks the first one, two, three, four, five, six, or more nucleotides of SEQ ID NO: 82).
337. The polynucleotide of any one of embodiments 285-315, wherein the 3′ UTR comprises the sequence of SEQ ID NO: 83, or a sequence with at least 80%, 85%, 90%, 95%, 96%, 97%, 98%, 99% or 100% identity to SEQ ID NO: 83, or a fragment thereof (e.g., a fragment that lacks the first one, two, three, four, five, six, or more nucleotides of SEQ ID NO: 83).
338. The polynucleotide of any one of embodiments 285-315, wherein the 3′ UTR comprises the sequence of SEQ ID NO: 84, or a sequence with at least 80%, 85%, 90%, 95%, 96%, 97%, 98%, 99% or 100% identity to SEQ ID NO: 84, or a fragment thereof (e.g., a fragment that lacks the first one, two, three, four, five, six, or more nucleotides of SEQ ID NO: 84).
339. The polynucleotide of any one of embodiments 285-315, wherein the 3′ UTR comprises the sequence of SEQ ID NO: 85, or a sequence with at least 80%, 85%, 90%, 95%, 96%, 97%, 98%, 99% or 100% identity to SEQ ID NO: 85, or a fragment thereof (e.g., a fragment that lacks the first one, two, three, four, five, six, or more nucleotides of SEQ ID NO: 85).
340. The polynucleotide of any one of embodiments 285-315, wherein the 3′ UTR comprises the sequence of SEQ ID NO: 86, or a sequence with at least 80%, 85%, 90%, 95%, 96%, 97%, 98%, 99% or 100% identity to SEQ ID NO: 86, or a fragment thereof (e.g., a fragment that lacks the first one, two, three, four, five, six, or more nucleotides of SEQ ID NO: 86).
341. The polynucleotide of any one of embodiments 285-315, wherein the 3′ UTR comprises the sequence of SEQ ID NO: 87, or a sequence with at least 80%, 85%, 90%, 95%, 96%, 97%, 98%, 99% or 100% identity to SEQ ID NO: 87, or a fragment thereof (e.g., a fragment that lacks the first one, two, three, four, five, six, or more nucleotides of SEQ ID NO: 87).
342. The polynucleotide of any one of embodiments 285-315, wherein the 3′ UTR comprises the sequence of SEQ ID NO: 94, or a sequence with at least 80%, 85%, 90%, 95%, 96%, 97%, 98%, 99% or 100% identity to SEQ ID NO: 94, or a fragment thereof (e.g., a fragment that lacks the first one, two, three, four, five, six, or more nucleotides of SEQ ID NO: 94).
343. The polynucleotide of any one of embodiments 285-315, wherein the 3′ UTR comprises the sequence of SEQ ID NO: 95, or a sequence with at least 80%, 85%, 90%, 95%, 96%, 97%, 98%, 99% or 100% identity to SEQ ID NO: 95, or a fragment thereof (e.g., a fragment that lacks the first one, two, three, four, five, six, or more nucleotides of SEQ ID NO: 95).
344. The polynucleotide of embodiment any one of embodiments 285-315, wherein the 3′ UTR comprises a micro RNA binding site, e.g., as described herein, and/or a TENT recruiting sequence, e.g., as described herein.
345. The polynucleotide of embodiment 344, wherein the miRNA binding site comprises a sequence of any one of SEQ ID NOs: 38-40, or wherein the TENT recruiting sequence comprises a sequence of SEQ ID NO: 91 or 92.
346. A polynucleotide encoding a polypeptide, wherein the polynucleotide comprises:

(a) a 5′-UTR (e.g., as described herein);

(b) a coding region comprising a stop element (e.g., as described herein); and

(c) a 3′ UTR comprising a core sequence with at least 80%, 85%, 90%, 95%, 96%, 97%, 98%, 99% or 100% identity to SEQ ID NO: 11 or a variant or a fragment thereof.

347. The polynucleotide of embodiment 346, wherein the 3′ UTR core sequence is disposed immediately downstream of the stop element of (b).
348. The polynucleotide of embodiment 347, wherein the 3′ UTR core sequence is disposed at the C terminus end of the polynucleotide.
349. The polynucleotide of any one of embodiments 346-348, wherein the 3′ UTR comprising a core sequence comprises a first flanking sequence.
350. The polynucleotide of any one of embodiments 346-349, wherein the 3′ UTR comprising a core sequence comprises a second flanking sequence.
351. The polynucleotide of embodiment 349 or 350, wherein the 3′ UTR comprising a core sequence comprises a first flanking sequence and a second flanking sequence.
352. The polynucleotide of any one of embodiments 349-351, wherein the first flanking sequence comprises a sequence of about 5-25, about 5-20, about 5-15, about 5-10, about 10-25, about 15-25, about 20-25 nucleotides.
353. The polynucleotide of any one of embodiments 349-351, wherein the first flanking sequence comprises a sequence of about 5, 6, 7, 8, 9, 10, 11, 12, 13, 14, 15, 16, 17, 18, 19, 20, 21, 22, 23, 24, or 25 nucleotides, e.g., 11 nucleotides.
354. The polynucleotide of any one of embodiments 349-351, wherein the second flanking sequence comprises a sequence of about 20-80, about 20-75, about 20-70, about 20-65, about 20-60, about 20-55, about 20-50, about 20-45, bout 20-40, about 20-35, about 20-30, about 20-25, about 25-80, about 30-80, about 35-80, about 40-80, about 45-80, about 50-80, about 55-80, about 60-80, about 65-80, about 70-80 or about 75-80 nucleotides.
355. The polynucleotide of any one of embodiments 349-351, wherein the second flanking sequence comprises a sequence of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, or 80 nucleotides, e.g., 39 nucleotides.
356. The polynucleotide of any one of embodiments 349-351, wherein the first flanking sequence is upstream of the core sequence.
357. The polynucleotide of any one of embodiments 349-351, wherein the first flanking sequence is downstream of the core sequence.
358. The polynucleotide of any one of embodiments 349-351, wherein the second flanking sequence is upstream of the core sequence.
359. The polynucleotide of any one of embodiments 349-351, wherein the second flanking sequence is downstream of the core sequence.
360. The polynucleotide of any one of embodiments 346-359, wherein the 3′ UTR comprises a fragment of SEQ ID NO: 11, e.g., a 5 nucleotide (nt), 10 nt, 15 nt, 20 nt, 25 nt, 30 nt, 35 nt, 40 nt, 45 nt, 50 nt, 55 nt, 60 nt, 65 nt, or 70 nt fragment of SEQ ID NO: 11.
361. The polynucleotide of any one of embodiments 346-360, wherein the 3′ UTR comprises 15-25 nt fragment comprising a 60 nt fragment of SEQ ID NO: 11.
362. The polynucleotide of any one of embodiments 346-361, wherein the 3′ UTR comprises the sequence of SEQ ID NO: 45.
363. The polynucleotide of any one of embodiments 346-361, wherein the 3′ UTR comprises the sequence of SEQ ID NO: 11.
364. The polynucleotide of any one of embodiments 346-363, wherein the 3′ UTR results in an increased half-life of the polynucleotide, e.g., about 1.5-10 fold increase in half-life of the polynucleotide, e.g., as measured by an assay that measures the half-life of a polynucleotide, e.g., an assay of any one of Examples disclosed herein.
365. The polynucleotide of embodiment 364, wherein the 3′ UTR results in a polynucleotide with a mean half-life score of greater than 10.
366. The polynucleotide of any one of embodiments 346-365, wherein the increase in half-life of the polynucleotide is compared to an otherwise similar polynucleotide which does not have a 3′ UTR, has a different 3′ UTR, or does not have the 3′ UTR of any one of embodiments 346-244.
367. The polynucleotide of any one of embodiments 346-363, wherein the 3′ UTR results in an increased level and/or activity, e.g., output, of the polypeptide encoded by the polynucleotide.
368. The polynucleotide of embodiment 248, wherein the increase in level and/or activity, e.g., output, of the polypeptide encoded by the polynucleotide is compared to an otherwise similar polynucleotide which does not have a 3′ UTR, has a different 3′ UTR, or does not have the 3′ UTR of any one of embodiments 346-363.
369. The polynucleotide of any one of embodiments 346-368, wherein the 3′ UTR comprises a micro RNA (miRNA) binding site, e.g., as described herein; and/or a TENT recruiting sequence, e.g., as described herein.
370. The polynucleotide of embodiment 369, wherein the 3′ UTR comprises a miRNA binding site of SEQ ID NO: 38, SEQ ID NO: 39, SEQ ID NO: 40 or a combination thereof, and/or wherein the TENT recruiting sequence comprises a sequence of SEQ ID NO: 91 or 92.
371. The polynucleotide of embodiment 369 or 370, wherein the 3′ UTR comprises a plurality of miRNA binding sites, e.g., 2, 3, 4, 5, 6, 7 or 8 miRNA binding sites.
372. The polynucleotide of embodiment 371, wherein the plurality of miRNA binding sites comprises the same or different miRNA binding sites.
373. The polynucleotide of any one of embodiments 346-372, wherein the 5′ UTR of (a) comprises a 5′ UTR sequence provided in Table 1 or a sequence with at least 80%, 85%, 90%, 95%, 96%, 97%, 98%, 99% or 100% identity to a 5′ UTR sequence provided in Table 1, or a fragment thereof (e.g., a fragment that lacks the first one, two, three, four, five, or six nucleotides of the 5′ UTR sequence provided in Table 1).
374. The polynucleotide of embodiment 373, wherein the 5′ UTR comprises a sequence with at least 80%, 85%, 90%, 95%, 96%, 97%, 98%, 99% or 100% identity to SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 41, SEQ ID NO: 42, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 88, SEQ ID NO: 89 or SEQ ID NO: 90, or a fragment thereof (e.g., a fragment that lacks the first one, two, three, four, five, or six nucleotides of any of the aforesaid sequences).
375. The polynucleotide of embodiment 373, wherein the 5′ UTR comprises a sequence with at least 80%, 85%, 90%, 95%, 96%, 97%, 98%, 99% or 100% identity to SEQ ID NO: 1, or a fragment thereof (e.g., a fragment that lacks the first one, two, three, four, five, six, or more nucleotides of SEQ ID NO: 2).
376. The polynucleotide of embodiment 373, wherein the 5′ UTR comprises a sequence with at least 80%, 85%, 90%, 95%, 96%, 97%, 98%, 99% or 100% identity to SEQ ID NO: 2, or a fragment thereof (e.g., a fragment that lacks the first one, two, three, four, five, six, or more nucleotides of SEQ ID NO: 2).
377. The polynucleotide of embodiment 373, wherein the 5′ UTR comprises a sequence with at least 80%, 85%, 90%, 95%, 96%, 97%, 98%, 99% or 100% identity to SEQ ID NO: 3, or a fragment thereof (e.g., a fragment that lacks the first one, two, three, four, five, six, or more nucleotides of SEQ ID NO: 3).
378. The polynucleotide of embodiment 373, wherein the 5′ UTR comprises a sequence with at least 80%, 85%, 90%, 95%, 96%, 97%, 98%, 99% or 100% identity to SEQ ID NO: 4, or a fragment thereof (e.g., a fragment that lacks the first one, two, three, four, five, six, or more nucleotides of SEQ ID NO: 4).
379. The polynucleotide of embodiment 373, wherein the 5′ UTR comprises a sequence with at least 80%, 85%, 90%, 95%, 96%, 97%, 98%, 99% or 100% identity to SEQ ID NO: 5, or a fragment thereof (e.g., a fragment that lacks the first one, two, three, four, five, six, or more nucleotides of SEQ ID NO: 5).
380. The polynucleotide of embodiment 373, wherein the 5′ UTR comprises a sequence with at least 80%, 85%, 90%, 95%, 96%, 97%, 98%, 99% or 100% identity to SEQ ID NO: 6, or a fragment thereof (e.g., a fragment that lacks the first one, two, three, four, five, six, or more nucleotides of SEQ ID NO: 6).
381. The polynucleotide of embodiment 373, wherein the 5′ UTR comprises a sequence with at least 80%, 85%, 90%, 95%, 96%, 97%, 98%, 99% or 100% identity to SEQ ID NO: 8, or a fragment thereof (e.g., a fragment that lacks the first one, two, three, four, five, six, or more nucleotides of SEQ ID NO: 8).
382. The polynucleotide of embodiment 373, wherein the 5′ UTR comprises a sequence with at least 80%, 85%, 90%, 95%, 96%, 97%, 98%, 99% or 100% identity to SEQ ID NO: 41, or a fragment thereof (e.g., a fragment that lacks the first one, two, three, four, five, six, or more nucleotides of SEQ ID NO: 41).
383. The polynucleotide of embodiment 373, wherein the 5′ UTR comprises a sequence with at least 80%, 85%, 90%, 95%, 96%, 97%, 98%, 99% or 100% identity to SEQ ID NO: 42, or a fragment thereof (e.g., a fragment that lacks the first one, two, three, four, five, six, or more nucleotides of SEQ ID NO: 42).
384. The polynucleotide of embodiment 373, wherein the 5′ UTR comprises the sequence of SEQ ID NO: 63 or a fragment thereof (e.g., a fragment that lacks the first one, two, three, four, five, or six nucleotides of SEQ ID NO: 63).
385. The polynucleotide of embodiment 373, wherein the 5′ UTR comprises the sequence of SEQ ID NO: 64 or a fragment thereof (e.g., a fragment that lacks the first one, two, three, four, five, or six nucleotides of SEQ ID NO: 64).
386. The polynucleotide of embodiment 373, wherein the 5′ UTR comprises the sequence of SEQ ID NO: 65 or a fragment thereof (e.g., a fragment that lacks the first one, two, three, four, five, or six nucleotides of SEQ ID NO: 65).
387. The polynucleotide of embodiment 373, wherein the 5′ UTR comprises the sequence of SEQ ID NO: 66 or a fragment thereof (e.g., a fragment that lacks the first one, two, three, four, five, or six nucleotides of SEQ ID NO: 66).
388. The polynucleotide of embodiment 373, wherein the 5′ UTR comprises the sequence of SEQ ID NO: 67 or a fragment thereof (e.g., a fragment that lacks the first one, two, three, four, five, or six nucleotides of SEQ ID NO: 67).
389. The polynucleotide of embodiment 373, wherein the 5′ UTR comprises the sequence of SEQ ID NO: 68 or a fragment thereof (e.g., a fragment that lacks the first one, two, three, four, five, or six nucleotides of SEQ ID NO: 68).
390. The polynucleotide of embodiment 373, wherein the 5′ UTR comprises the sequence of SEQ ID NO: 69 or a fragment thereof (e.g., a fragment that lacks the first one, two, three, four, five, or six nucleotides of SEQ ID NO: 69).
391. The polynucleotide of embodiment 373, wherein the 5′ UTR comprises the sequence of SEQ ID NO: 70 or a fragment thereof (e.g., a fragment that lacks the first one, two, three, four, five, or six nucleotides of SEQ ID NO: 70).
392. The polynucleotide of embodiment 373, wherein the 5′ UTR comprises the sequence of SEQ ID NO: 70 or a fragment thereof (e.g., a fragment that lacks the first one, two, three, four, five, or six nucleotides of SEQ ID NO: 70).
393. The polynucleotide of embodiment 373, wherein the 5′ UTR comprises the sequence of SEQ ID NO: 71 or a fragment thereof (e.g., a fragment that lacks the first one, two, three, four, five, or six nucleotides of SEQ ID NO: 71).
394. The polynucleotide of embodiment 373, wherein the 5′ UTR comprises the sequence of SEQ ID NO: 72 or a fragment thereof (e.g., a fragment that lacks the first one, two, three, four, five, or six nucleotides of SEQ ID NO: 72).
395. The polynucleotide of embodiment 373, wherein the 5′ UTR comprises the sequence of SEQ ID NO: 73 or a fragment thereof (e.g., a fragment that lacks the first one, two, three, four, five, or six nucleotides of SEQ ID NO: 73).
396. The polynucleotide of embodiment 373, wherein the 5′ UTR comprises the sequence of SEQ ID NO: 74 or a fragment thereof (e.g., a fragment that lacks the first one, two, three, four, five, or six nucleotides of SEQ ID NO: 74).
397. The polynucleotide of embodiment 373, wherein the 5′ UTR comprises the sequence of SEQ ID NO: 75 or a fragment thereof (e.g., a fragment that lacks the first one, two, three, four, five, or six nucleotides of SEQ ID NO: 75).
398. The polynucleotide of embodiment 373, wherein the 5′ UTR comprises the sequence of SEQ ID NO: 76 or a fragment thereof (e.g., a fragment that lacks the first one, two, three, four, five, or six nucleotides of SEQ ID NO: 76).
399. The polynucleotide of embodiment 373, wherein the 5′ UTR comprises the sequence of SEQ ID NO: 77 or a fragment thereof (e.g., a fragment that lacks the first one, two, three, four, five, or six nucleotides of SEQ ID NO: 77).
400. The polynucleotide of embodiment 373, wherein the 5′ UTR comprises the sequence of SEQ ID NO: 78 or a fragment thereof (e.g., a fragment that lacks the first one, two, three, four, five, or six nucleotides of SEQ ID NO: 78).
401. The polynucleotide of embodiment 373, wherein the 5′ UTR comprises the sequence of SEQ ID NO: 88 or a fragment thereof (e.g., a fragment that lacks the first one, two, three, four, five, or six nucleotides of SEQ ID NO: 88).
402. The polynucleotide of embodiment 373, wherein the 5′ UTR comprises the sequence of SEQ ID NO: 89 or a fragment thereof (e.g., a fragment that lacks the first one, two, three, four, five, or six nucleotides of SEQ ID NO: 89).
403. The polynucleotide of embodiment 373, wherein the 5′ UTR comprises the sequence of SEQ ID NO: 90 or a fragment thereof (e.g., a fragment that lacks the first one, two, three, four, five, or six nucleotides of SEQ ID NO: 90).
404. The polynucleotide of any one of embodiments 346-372, wherein the stop element of (b) comprises a stop element sequence provided in Table 3.
405. The polynucleotide of embodiment 404, wherein the coding region of (b) comprises a stop element chosen from a stop element provided in Table 3, e.g., SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO:36, SEQ ID NO; 62, SEQ ID NO: 93 or SEQ ID NO: 96.
406. The polynucleotide of embodiment 404, wherein the stop element comprises the sequence of SEQ ID NO: 26.
407. The polynucleotide of embodiment 404, wherein the stop element comprises the sequence of SEQ ID NO: 27.
408. The polynucleotide of embodiment 404, wherein the stop element comprises the sequence of SEQ ID NO: 28.
409. The polynucleotide of embodiment 404, wherein the stop element comprises the sequence of SEQ ID NO: 29.
410. The polynucleotide of embodiment 404, wherein the stop element comprises the sequence of SEQ ID NO: 30.
411. The polynucleotide of embodiment 404, wherein the stop element comprises the sequence of SEQ ID NO: 31.
412. The polynucleotide of embodiment 404, wherein the stop element comprises the sequence of SEQ ID NO: 32.
413. The polynucleotide of embodiment 404, wherein the stop element comprises the sequence of SEQ ID NO: 33.
414. The polynucleotide of embodiment 404, wherein the stop element comprises the sequence of SEQ ID NO: 34.
415. The polynucleotide of embodiment 404, wherein the stop element comprises the sequence of SEQ ID NO: 35.
416. The polynucleotide of embodiment 404, wherein the stop element comprises the sequence of SEQ ID NO: 36.
417. The polynucleotide of embodiment 404, wherein the stop element comprises the sequence of SEQ ID NO: 62.
418. The polynucleotide of embodiment 404, wherein the stop element comprises the sequence of SEQ ID NO: 93.
419. The polynucleotide of embodiment 404, wherein the stop element comprises the sequence of SEQ ID NO: 96.
420. The polynucleotide of embodiment 404, wherein the coding region of (b) comprises a stop element comprising the consensus sequence of SEQ ID NO: 37.
421. The polynucleotide of any one of embodiments 346-372, wherein:

(i) the 5′ UTR of (a) comprises a 5′ UTR sequence provided in Table 1 or a sequence with at least 80%, 85%, 90%, 95%, 96%, 97%, 98%, 99% or 100% identity to a 5′ UTR sequence provided in Table 1, or a fragment thereof (e.g., a fragment that lacks the first one, two, three, four, five, or six nucleotides of the 5′ UTR sequence provided in Table 1); and

(ii) the stop element of (b) comprises a stop element provided in Table 3.

422. The polynucleotide of embodiment 421, wherein the 5′ UTR comprises a sequence with at least 80%, 85%, 90%, 95%, 96%, 97%, 98%, 99% or 100% identity to SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 41, SEQ ID NO: 42, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 88, SEQ ID NO: 89 or SEQ ID NO: 90, or a fragment thereof (e.g., a fragment that lacks the first one, two, three, four, five, or six nucleotides of any of the aforesaid sequences).
423. The polynucleotide of embodiment 421, wherein the 5′ UTR comprises a sequence with at least 80%, 85%, 90%, 95%, 96%, 97%, 98%, 99% or 100% identity to SEQ ID NO: 1, or a fragment thereof (e.g., a fragment that lacks the first one, two, three, four, five, six, or more nucleotides of SEQ ID NO: 2).
424. The polynucleotide of embodiment 421, wherein the 5′ UTR comprises a sequence with at least 80%, 85%, 90%, 95%, 96%, 97%, 98%, 99% or 100% identity to SEQ ID NO: 2, or a fragment thereof (e.g., a fragment that lacks the first one, two, three, four, five, six, or more nucleotides of SEQ ID NO: 2).
425. The polynucleotide of embodiment 421, wherein the 5′ UTR comprises a sequence with at least 80%, 85%, 90%, 95%, 96%, 97%, 98%, 99% or 100% identity to SEQ ID NO: 3, or a fragment thereof (e.g., a fragment that lacks the first one, two, three, four, five, six, or more nucleotides of SEQ ID NO: 3).
426. The polynucleotide of embodiment 421, wherein the 5′ UTR comprises a sequence with at least 80%, 85%, 90%, 95%, 96%, 97%, 98%, 99% or 100% identity to SEQ ID NO: 4, or a fragment thereof (e.g., a fragment that lacks the first one, two, three, four, five, six, or more nucleotides of SEQ ID NO: 4).
427. The polynucleotide of embodiment 421, wherein the 5′ UTR comprises a sequence with at least 80%, 85%, 90%, 95%, 96%, 97%, 98%, 99% or 100% identity to SEQ ID NO: 5, or a fragment thereof (e.g., a fragment that lacks the first one, two, three, four, five, six, or more nucleotides of SEQ ID NO: 5).
428. The polynucleotide of embodiment 421, wherein the 5′ UTR comprises a sequence with at least 80%, 85%, 90%, 95%, 96%, 97%, 98%, 99% or 100% identity to SEQ ID NO: 6, or a fragment thereof (e.g., a fragment that lacks the first one, two, three, four, five, six, or more nucleotides of SEQ ID NO: 6).
429. The polynucleotide of embodiment 421, wherein the 5′ UTR comprises a sequence with at least 80%, 85%, 90%, 95%, 96%, 97%, 98%, 99% or 100% identity to SEQ ID NO: 8, or a fragment thereof (e.g., a fragment that lacks the first one, two, three, four, five, six, or more nucleotides of SEQ ID NO: 8).
430. The polynucleotide of embodiment 421, wherein the 5′ UTR comprises a sequence with at least 80%, 85%, 90%, 95%, 96%, 97%, 98%, 99% or 100% identity to SEQ ID NO: 41, or a fragment thereof (e.g., a fragment that lacks the first one, two, three, four, five, six, or more nucleotides of SEQ ID NO: 41).
431. The polynucleotide of embodiment 421, wherein the 5′ UTR comprises a sequence with at least 80%, 85%, 90%, 95%, 96%, 97%, 98%, 99% or 100% identity to SEQ ID NO: 42, or a fragment thereof (e.g., a fragment that lacks the first one, two, three, four, five, six, or more nucleotides of SEQ ID NO: 42).
432. The polynucleotide of embodiment 421, wherein the 5′ UTR comprises the sequence of SEQ ID NO: 63 or a fragment thereof (e.g., a fragment that lacks the first one, two, three, four, five, or six nucleotides of SEQ ID NO: 63).
433. The polynucleotide of embodiment 421, wherein the 5′ UTR comprises the sequence of SEQ ID NO: 64 or a fragment thereof (e.g., a fragment that lacks the first one, two, three, four, five, or six nucleotides of SEQ ID NO: 64).
434. The polynucleotide of embodiment 421, wherein the 5′ UTR comprises the sequence of SEQ ID NO: 65 or a fragment thereof (e.g., a fragment that lacks the first one, two, three, four, five, or six nucleotides of SEQ ID NO: 65).
435. The polynucleotide of embodiment 421, wherein the 5′ UTR comprises the sequence of SEQ ID NO: 66 or a fragment thereof (e.g., a fragment that lacks the first one, two, three, four, five, or six nucleotides of SEQ ID NO: 66).
436. The polynucleotide of embodiment 421, wherein the 5′ UTR comprises the sequence of SEQ ID NO: 67 or a fragment thereof (e.g., a fragment that lacks the first one, two, three, four, five, or six nucleotides of SEQ ID NO: 67).
437. The polynucleotide of embodiment 421, wherein the 5′ UTR comprises the sequence of SEQ ID NO: 68 or a fragment thereof (e.g., a fragment that lacks the first one, two, three, four, five, or six nucleotides of SEQ ID NO: 68).
438. The polynucleotide of embodiment 421, wherein the 5′ UTR comprises the sequence of SEQ ID NO: 69 or a fragment thereof (e.g., a fragment that lacks the first one, two, three, four, five, or six nucleotides of SEQ ID NO: 69).
439. The polynucleotide of embodiment 421, wherein the 5′ UTR comprises the sequence of SEQ ID NO: 70 or a fragment thereof (e.g., a fragment that lacks the first one, two, three, four, five, or six nucleotides of SEQ ID NO: 70).
440. The polynucleotide of embodiment 421, wherein the 5′ UTR comprises the sequence of SEQ ID NO: 70 or a fragment thereof (e.g., a fragment that lacks the first one, two, three, four, five, or six nucleotides of SEQ ID NO: 70).
441. The polynucleotide of embodiment 421, wherein the 5′ UTR comprises the sequence of SEQ ID NO: 71 or a fragment thereof (e.g., a fragment that lacks the first one, two, three, four, five, or six nucleotides of SEQ ID NO: 71).
442. The polynucleotide of embodiment 421, wherein the 5′ UTR comprises the sequence of SEQ ID NO: 72 or a fragment thereof (e.g., a fragment that lacks the first one, two, three, four, five, or six nucleotides of SEQ ID NO: 72).
443. The polynucleotide of embodiment 421, wherein the 5′ UTR comprises the sequence of SEQ ID NO: 73 or a fragment thereof (e.g., a fragment that lacks the first one, two, three, four, five, or six nucleotides of SEQ ID NO: 73).
444. The polynucleotide of embodiment 421, wherein the 5′ UTR comprises the sequence of SEQ ID NO: 74 or a fragment thereof (e.g., a fragment that lacks the first one, two, three, four, five, or six nucleotides of SEQ ID NO: 74).
445. The polynucleotide of embodiment 421, wherein the 5′ UTR comprises the sequence of SEQ ID NO: 75 or a fragment thereof (e.g., a fragment that lacks the first one, two, three, four, five, or six nucleotides of SEQ ID NO: 75).
446. The polynucleotide of embodiment 421, wherein the 5′ UTR comprises the sequence of SEQ ID NO: 76 or a fragment thereof (e.g., a fragment that lacks the first one, two, three, four, five, or six nucleotides of SEQ ID NO: 76).
447. The polynucleotide of embodiment 421, wherein the 5′ UTR comprises the sequence of SEQ ID NO: 77 or a fragment thereof (e.g., a fragment that lacks the first one, two, three, four, five, or six nucleotides of SEQ ID NO: 77).
448. The polynucleotide of embodiment 421, wherein the 5′ UTR comprises the sequence of SEQ ID NO: 78 or a fragment thereof (e.g., a fragment that lacks the first one, two, three, four, five, or six nucleotides of SEQ ID NO: 87).
449. The polynucleotide of embodiment 421, wherein the 5′ UTR comprises the sequence of SEQ ID NO: 88 or a fragment thereof (e.g., a fragment that lacks the first one, two, three, four, five, or six nucleotides of SEQ ID NO: 88).
450. The polynucleotide of embodiment 421, wherein the 5′ UTR comprises the sequence of SEQ ID NO: 89 or a fragment thereof (e.g., a fragment that lacks the first one, two, three, four, five, or six nucleotides of SEQ ID NO: 89).
451. The polynucleotide of embodiment 421, wherein the 5′ UTR comprises the sequence of SEQ ID NO: 90 or a fragment thereof (e.g., a fragment that lacks the first one, two, three, four, five, or six nucleotides of SEQ ID NO: 90).
452. The polynucleotide of any one of embodiments 421-451, wherein the coding region of (b) comprises a stop element chosen from a stop element provided in Table 3, e.g., SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO:36, SEQ ID NO: 37, SEQ ID NO: 56, SEQ ID NO: 57, SEQ ID NO: 62, SEQ ID NO: 93 or SEQ ID NO: 96.
453. The polynucleotide of embodiment 452, wherein the stop element comprises the sequence of SEQ ID NO: 26.
454. The polynucleotide of embodiment 452, wherein the stop element comprises the sequence of SEQ ID NO: 27.
455. The polynucleotide of embodiment 452, wherein the stop element comprises the sequence of SEQ ID NO: 28.
456. The polynucleotide of embodiment 452, wherein the stop element comprises the sequence of SEQ ID NO: 29.
457. The polynucleotide of embodiment 452, wherein the stop element comprises the sequence of SEQ ID NO: 30.
458. The polynucleotide of embodiment 452, wherein the stop element comprises the sequence of SEQ ID NO: 31.
459. The polynucleotide of embodiment 452, wherein the stop element comprises the sequence of SEQ ID NO: 32.
460. The polynucleotide of embodiment 452, wherein the stop element comprises the sequence of SEQ ID NO: 33.
461. The polynucleotide of embodiment 452, wherein the stop element comprises the sequence of SEQ ID NO: 34.
462. The polynucleotide of embodiment 452, wherein the stop element comprises the sequence of SEQ ID NO: 35.
463. The polynucleotide of embodiment 452, wherein the stop element comprises the sequence of SEQ ID NO: 36.
464. The polynucleotide of embodiment 452, wherein the stop element comprises the sequence of SEQ ID NO: 62.
465. The polynucleotide of embodiment 452, wherein the stop element comprises the sequence of SEQ ID NO: 93.
466. The polynucleotide of embodiment 452, wherein the stop element comprises the sequence of SEQ ID NO: 96.
467. The polynucleotide of embodiment 452, wherein the coding region of (b) comprises a stop element comprising the consensus sequence of SEQ ID NO: 37, SEQ ID NO: 56 or SEQ ID NO: 57.
468. The polynucleotide of any one of the preceding embodiments, wherein the coding region of the polynucleotide comprises a sequence encoding a therapeutic payload or a prophylactic payload.
469. The polynucleotide of embodiment 468, wherein the therapeutic payload or prophylactic payload comprises a secreted protein, a membrane-bound protein; or an intercellular protein.
470. The polynucleotide of embodiment 469, wherein the therapeutic payload or prophylactic payload is chosen from a cytokine, an antibody, a vaccine (e.g., an antigen, an immunogenic epitope), a receptor, an enzyme, a hormone, a transcription factor, a ligand, a membrane transporter, a structural protein, a nuclease, or a component, variant or fragment (e.g., a biologically active fragment) thereof.
471. The polynucleotide of embodiment 469, wherein the therapeutic payload or prophylactic payload comprises a cytokine, or a variant or fragment (e.g., a biologically active fragment) thereof.
472. The polynucleotide of embodiment 469, wherein the therapeutic payload or prophylactic payload comprises an antibody or a variant or fragment (e.g., a biologically active fragment) thereof.
473. The polynucleotide of embodiment 469, wherein the therapeutic payload or prophylactic payload comprises a vaccine (e.g., an antigen, an immunogenic epitope), or a component, variant or fragment (e.g., a biologically active fragment) thereof.
474. The polynucleotide of any one of embodiments 468-473, wherein the therapeutic payload or prophylactic payload comprises a protein or peptide.
475. The polynucleotide of any one of the preceding embodiments, wherein a polynucleotide comprising a 5′ UTR of SEQ ID NO: 1, a coding region comprising a stop element of SEQ ID NO: 28 and a 3′ UTR of SEQ ID NO: 11, results in an increase in the level and/or activity of the polypeptide encoded by the polynucleotide.
476. The polynucleotide of embodiment 475, wherein the increase in level and/or activity of the polypeptide is about 1.2-10-fold.
477. The polynucleotide of embodiment 475 or 476, wherein the increase in level and/or activity of the polypeptide is measured by an assay which measures the activity of a polypeptide, e.g., an assay of Example 18.
478. The polynucleotide of any one of embodiments 1-474, wherein a polynucleotide comprising a 5′ UTR of SEQ ID NO: 1, SEQ ID NO: 41 or SEQ ID NO: 42 and a 3′ stabilizing region comprising an inverted thymidine (idT), results in an increase in the level and/or activity, e.g., expression, of a polypeptide encoded by the polynucleotide.
479. The polynucleotide of embodiment 478, wherein the increase in expression of the polypeptide is about 1.2-10 fold, e.g., as measured by an assay which measures the expression of a polypeptide, e.g., an immunoblot, an ELISA or flow cytometry, e.g., an assay described in any one of Examples disclosed herein.
480. The polynucleotide of embodiment 478, wherein the increase in activity of the polypeptide is about 1.2-10-fold, e.g., as measured by an assay which measures the activity of a polypeptide, e.g., an assay that tracks the kinetics of the formation of a metabolite.
481. The polynucleotide of embodiment 479 or 480, wherein the increase in level and/or activity of the polypeptide is compared to an otherwise similar polypeptide encoded by a polynucleotide that does not have a 5′ UTR, a 3′ UTR, a stop element and/or a 3′ stabilizing region described herein.
482. The polynucleotide of any one of the preceding embodiments, further comprising at least one 5′ cap structure, e.g., as described herein.
483. The polynucleotide of embodiment 482, wherein the 5′ cap structure comprises the sequence GG, wherein the underlined, italicized G is an inverted G nucleotide followed by a 5′-5′-triphosphate group.
484. The polynucleotide of embodiment 483, wherein the 5′ cap structure comprises the sequence GA, wherein the underlined, italicized G is an inverted G nucleotide followed by a 5′-5′-triphosphate group.
485. The polynucleotide of embodiment 483, wherein the 5′ cap structure comprises the sequence GGA, wherein the underlined, italicized G is an inverted G nucleotide followed by a 5′-5′-triphosphate group.
486. The polynucleotide of any one of the preceding embodiments, further comprising a 3′ stabilizing region, e.g., a stabilized tail e.g., as described herein.
487. The polynucleotide of embodiment 486, wherein the 3′ stabilizing region comprises a poly A tail, e.g., a poly A tail comprising 80-150, e.g., 120, adenines (SEQ ID NO: 123), optionally wherein the poly A tail comprises one or more non-adenosine residues, e.g., one or more guanosines.
488. The polynucleotide of embodiment 486 or 487, wherein the poly A tail comprises a UCUAG sequence (SEQ ID NO: 44).
489. The polynucleotide of embodiment 488, wherein the poly A tail comprises about 80-120, e.g., 100, adenines upstream of SEQ ID NO: 44.
490. The polynucleotide of embodiment 488 or 489, wherein the poly A tail comprises about 1-40, e.g., 20, adenines downstream of SEQ ID NO: 44.
491. The polynucleotide of any one of embodiments 486-490, wherein the 3′ stabilizing region comprises at least one alternative nucleoside.
492. The polynucleotide of embodiment 491, wherein the alternative nucleoside is an inverted thymidine (idT).
493. The polynucleotide of embodiment 491 or 492, wherein the alternative nucleoside is disposed at the 3′ end of the 3′ stabilizing region.
494. The polynucleotide of any one of embodiments 486-493, wherein the 3′ stabilizing region comprises a structure of Formula VII:

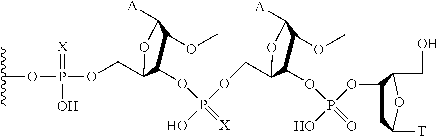

or a salt thereof, wherein each X is independently O or S, and A represents adenine and T represents thymine.
495. The polynucleotide of any one of the preceding embodiments, wherein the polynucleotide comprises an mRNA.
496. The polynucleotide of embodiment 495, wherein the mRNA comprises at least one chemical modification.
497. The polynucleotide of embodiment 495 or 496, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.
498. The polynucleotide of embodiment 497, wherein the chemical modification is selected from the group consisting of pseudouridine, N1-methylpseudouridine, 5-methylcytosine, 5-methoxyuridine, and a combination thereof.
499. The polynucleotide of embodiment 497, wherein the chemical modification is N1-methylpseudouridine.
500. The polynucleotide of embodiment 497, wherein the mRNA comprises fully modified N1-methylpseudouridine.
501. A lipid nanoparticle (LNP) composition comprising a polynucleotide of any one of the preceding embodiments.
502. A pharmaceutical composition comprising the LNP composition of embodiment 501.
503. A cell comprising the LNP composition of embodiment 501 or 502.
504. The cell of embodiment 503, which has been contacted with the LNP composition.
505. The cell of embodiment 503 or 504, which is maintained under conditions sufficient to allow for expression of the polynucleotide or polypeptide encoded by the polynucleotide.
506. A method of increasing expression of a payload, e.g., a therapeutic payload or a prophylactic payload in a cell, comprising administering to the cell the LNP composition of embodiment 501 or 502.
507. A method of delivering the LNP composition of embodiment 501 or 502, to a cell.
508. The method of embodiment 507, comprising contacting the cell in vitro, in vivo or ex vivo with the LNP composition.
509. A method of delivering an LNP composition of embodiment 501 or 502, to a subject having a disease or disorder, e.g., as described herein.
510. A method of modulating an immune response in a subject, comprising administering to the subject in need thereof an effective amount of an LNP composition of embodiment 501 or 502.
511. A method of treating, preventing, or preventing a symptom of, a disease or disorder comprising administering to a subject in need thereof an effective amount of an LNP composition of embodiment 501 or 502.
512. The method, or the LNP composition of any one of embodiments 501-511, wherein the LNP composition comprises: (i) an ionizable lipid, e.g., an amino lipid; (ii) a sterol or other structural lipid; (iii) a non-cationic helper lipid or phospholipid; and (iv) a PEG-lipid, e.g., as described herein.
513. The method, or the LNP composition of embodiment 512, wherein the ionizable lipid comprises an amino lipid.
514. The method, or the LNP composition of embodiment 512 or 513, wherein the ionizable lipid comprises a compound of any of Formulae described herein, e.g., Formulae (I), (IA), (IB), (IC), (II), (IIa), (IIb), (IIc), (IId), (IIe), (IIf), (IIg), (III), (IIIa1), (IIIa2), (IIIa3), (IIIa4), (IIIa5), (IIIa6), (IIIa7), or (IIIa8).
515. The method, or the LNP composition of any one of embodiments 512-514, wherein the ionizable lipid comprises a compound of Formula (I).
516. The method, or the LNP composition of any one of embodiments 512-514, wherein the ionizable lipid comprises a compound of Formula (IC).
517. The method, or the LNP composition of any one of embodiments 512-514, wherein the ionizable lipid comprises a compound of Formula (IIa).
518. The method, or the LNP composition of any one of embodiments 512-514, wherein the ionizable lipid comprises a compound of Formula (IIe).
519. The method, or the LNP composition of any one of embodiments 512-518, wherein the non-cationic helper lipid or phospholipid comprises a compound selected from the group consisting of DSPC, DPPC, or DOPC.
520. The method, or the LNP composition of any one of embodiments 512-519, wherein the phospholipid is DSPC, e.g., a variant of DSPC, e.g., a compound of Formula (IV).
521. The method, or the LNP composition of any one of embodiments 512-519, wherein the structural lipid is chosen from alpha-tocopherol, β-sitosterol or cholesterol.
522. The method, or the LNP composition of any one of embodiments 512-520, wherein the structural lipid is alpha-tocopherol.
523. The method, or the LNP composition of any one of embodiments 512-520, wherein the structural lipid is p-sitosterol.
524. The method, or the LNP composition of any one of embodiments 512-521, wherein the structural lipid is cholesterol.
525. The method, or the LNP composition of any one of embodiments 512-524, wherein the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.
526. The method, or the LNP composition of any one of embodiments 512-525, wherein the PEG lipid is selected from the group consisting of PEG-c-DOMG, PEG-DMG, PEG-DLPE, PEG-DMPE, PEG-DPPC and PEG-DSPE lipid.
527. The method, or the LNP composition of any one of embodiments 512-525, wherein the PEG-lipid is PEG-DMG.
528. The method, or the LNP composition of any one of embodiments 512-525, wherein the PEG lipid is a compound chosen from: Formula (V), Formula (VI-A), Formula (VI-B), Formula (VI-C) or Formula (VI-D).
529. The method, or the LNP composition of any one of embodiments 512-525 or 528, wherein the PEG-lipid is a compound of Formula (VI-A).
530. The method, or the LNP composition of any one of embodiments 512-525 or 528, wherein the PEG-lipid is a compound of Formula (VI-B).
531. The method, or the LNP composition of any one of embodiments 512-530, wherein the LNP comprises a molar ratio of about 20-60% ionizable lipid:5-25% phospholipid:25-55% cholesterol; and 0.5-15% PEG lipid.
532. The method, or the LNP composition of any one of embodiments 512-531, wherein the LNP comprises a molar ratio of about 50% ionizable lipid:about 10% phospholipid:about 38.5% cholesterol; and about 1.5% PEG lipid.
533. The method, or the LNP composition of any one of embodiments 512-532, wherein the LNP comprises a molar ratio of about 49.83% ionizable lipid:about 9.83% phospholipid:about 30.33% cholesterol; and about 2.0% PEG lipid.
534. The method, or the LNP composition of any one of embodiments 501-533, wherein the LNP or system is formulated for intravenous, subcutaneous, intramuscular, intranasal, intraocular, rectal, pulmonary or oral delivery.
535. The method, or the LNP composition of any one of embodiments 501-534, wherein the subject is a mammal, e.g., a human.
536. The method, or the LNP composition of any one of embodiments 501-535, wherein the subject has a disease or disorder disclosed herein.

### BRIEF DESCRIPTIONS OF THE DRAWINGS

FIG. 1 is a graph showing GFP fluorescence from GFP protein encoded by mRNA constructs having either the A11 reference 5′ UTR or the A1 5′ UTR.

FIGS. 2A-2C depict ffLuc activity in vivo in mice administered LNP formulated ffLuc mRNA. The mRNA constructs either had the A11 reference 5′ UTR or the A1 5′ UTR. FIG. 2A shows total flux at 6 hours post administration. FIG. 2B shows total flux at 48 hours post-administration. FIG. 2C shows the combined ffLuc activity observed at both timepoints.

FIGS. 3A-3B are graphs depicting target protein expression in rats administered LNP formulated target mRNA having the indicated 5′ UTRs. FIG. 3A shows target protein expression at the indicated time points. FIG. 3B shows total target protein expression as measured by the area under the curve (AUC).

FIGS. 4A-4C are graphs depicting median target protein expression in HepatoPacs seeded with hepatocytes from rats (FIG. 4A), rhesus macaque (FIG. 4B) or human primary hepatocytes (FIG. 4C).

FIGS. 5A-5D are graphs showing expression of a target protein in B cells or T cells from human PBMCs. Human PBMCs were contacted with LNP formulated mRNA encoding a target protein. The mRNA constructs either had the A11 reference 5′ UTR or the A1 5′ UTR. FIGS. 5A-5B show target protein expression in T cells. FIGS. 5C-5D show target protein expression in B cells.

FIG. 6 is a graph showing expression of a target protein associated with a rare disease. Hep3B cells were transfected with mRNA constructs encoding the target protein. mRNA constructs comprising two versions (v1, v2) of the ORF sequence were used. The mRNA constructs had the A1 5′ UTR or the A11 reference 5′ UTR.

FIG. 7 is a graph showing in vivo protein expression from a construct having a modified A1 5′ UTR sequence (A3) or a construct having the reference A11 5′ UTR.

FIG. 8 shows the output of an in vitro high-throughput 3′ UTR screen for mRNA half-life extension. The left panel shows changes in relative abundance of 3′ UTR sequences over the assessed time course. Data points represent the mean and standard deviation of all ORFs and cell types. The B1 sequence had the highest half-life score of all assessed sequences. Right panel shows a histogram of half-life scores. The prominent left-hand tail indicates that there are more 3′ UTR sequences that shorten than extend half-life. The blue and orange ticks represent the B10 reference 3′ UTR and B1 3′ UTR.

FIG. 9 shows the outline of the 3′ UTR bakeoff and the ORFs and cell types used. FIG. 9A discloses “KDEL” as SEQ ID NO: 130.

FIGS. 10A-10C show the results of the 3′ UTR bakeoff. FIG. 10A is a graph showing the relationship between inferred mRNA half-life and overall expression for cytosolic mRNAs encoding a green fluoresce protein bearing different 3′ UTRs; points in red used 3′ UTRs derived from the high-throughput 3′ UTR screen for half-life extension. All values are normalized to an in-plate v1.1 3′ UTR control. FIG. 10B shows similar data as in FIG. 10A, but comparing inferred translation efficiency to AUC expression. FIG. 10C is a graph showing data from an IncuCyte expression experiment for an mRNA encoding a green fluoresce protein bear either the B10 reference 3′ UTR or the B1 3′ UTR.

FIGS. 11A-11F show mRNA half-life for mRNAs having different stop elements. FIG. 11A shows the distribution of median natural mRNA half-lives for mRNAs bearing different stop codons plus 2 downstream nucleotides of context. Each point represents a different 5nt sequence (e.g. UAAGC, where the stop codon itself is underlined). FIG. 11B shows examples of median mRNA half-lives for mRNAs with different stop codon cassettes. FIG. 11C shows data from an IncuCyte expression experiment for an mRNA encoding a red fluoresce protein comparing an mRNA bearing the B10 reference 3′ UTR having the C1 stop element (see black line) to an mRNA with the C4 stop element (SEQ ID NO: 29, UAAAGCUAA; see red line). Note codons in figure legends and tables are based on DNA nomenclature and use “T” instead of “U”. FIG. 11D shows the median natural mRNA half-life in HeLa cells for mRNAs containing each potential nucleotide at several positions relative to the UAA stop codon. This analytical approach was used to generate stop element C7 and C6 in Table 3. FIG. 11D discloses SEQ ID NO: 129. FIG. 11E shows similar data as in FIG. 11D but for the UAG stop codon. FIG. 11F shows similar data as in FIG. 11D but for the UGA stop codon.

FIG. 12 shows expression of a target protein associated with a rare disease in Hep G2 cells. The target protein is encoded by mRNA constructs having different stop elements: C5, C4, C11, C3, or a reference stop element (C1). Target protein expression was evaluated with immunoblotting and is plotted over time.

FIG. 13 is a graph showing the expression of a target protein encoded by mRNA constructs having various stop elements in the 3′ UTR. The mRNA constructs used comprised the following 3′ UTR and stop element sequence: SEQ ID NO: 25 (B16 control), SEQ ID NO: 59 (3′ UTR with C10 stop element), SEQ ID NO: 60 (3′ UTR with C7 stop element) and SEQ ID NO: 61 (3′ UTR with C8 stop element).

FIGS. 14A-14B show the in vivo expression of an immune checkpoint protein encoded by mRNA constructs having the specified mRNA elements in the figure. FIG. 14A shows the level of the immune checkpoint protein in the spleen of mice intravenously injected with 0.5 mg/kg of LNP formulated mRNAs encoding the immune checkpoint protein. FIG. 14B shows the level of the immune checkpoint protein in the liver of mice intravenously injected with 0.5 mg/kg of LNP formulated mRNAs encoding the immune checkpoint protein.

FIG. 15 shows % immune checkpoint protein+cells among CD11c+MHCII+ cells from mice administered 0.5 mg/kg of LNP formulated mRNAs encoding the immune checkpoint protein and having the mRNA elements specified.

FIGS. 16A-16C are graphs depicting luciferase or target protein expression encoded by mRNA constructs having the A1 5′ UTR (together with a cap comprising the sequence GA), the B1 3′ UTR or both. FIG. 16A shows expression in the spleen and FIG. 16B shows expression in the liver. FIG. 16C shows target protein expression in the serum.

FIG. 17 depicts expression of a target protein in human bronchial epithelial cells. The target protein was encoded by an mRNA having various elements shown in the figure. Two different open reading frames (ORFs) encoding the target protein were used in this experiment. The cells were transfected with the mRNAs and activity of the target protein was measured.

FIG. 18A is a schematic depiction of the design of an exemplary mRNA construct described herein.

FIG. 18B is a graph showing the expression of a red fluorescence protein in Hela cells. The target protein is encoded by mRNA constructs having different stop elements: C1, C5, C7, and C9.

FIGS. 18C-18D is a graph showing the expression of a green fluorescence protein in Hela cells. The target protein is encoded by mRNA constructs having different stop elements: C1, C5, C7, and C9.

FIG. 18E is a graph showing the expression of a red fluorescence protein in HEK293 cells. The target protein is encoded by mRNA constructs having different stop elements: C1, C5, C7, and C9.

FIGS. 18F-18G is a graph showing the expression of a green fluorescence protein in HEK293 cells. The target protein is encoded by mRNA constructs having different stop elements: C1, C5, C7, and C9.

FIGS. 18H and 18I are plots depicting the readthrough percentage rate of the green fluorescent protein in HeLa and HEK293 cells, respectively. The target protein is encoded by mRNA constructs having different stop elements: C1, C3, C5, C7, and C9.

FIGS. 19A-19C are plots depicting expression of a target protein in HeLa cells at 24 and 48 hours. The target protein is encoded by mRNA constructs having different stop elements: C1, C5, C10, C7, C8, and C9.

FIGS. 19D-19F are plots depicting expression of a target protein in HEK293 cells at 24 and 48 hours. The target protein is encoded by mRNA constructs having different stop elements: C1, C5, C10, C7, C8, and C9.

FIGS. 20A-20B are plots depicting expression of a target protein in HeLa and Hep3b cells at 24 and 48 hours. The target protein is encoded by mRNA constructs having different stop elements: C1, C5, C10, C7, C8, and C9.

FIG. 21A is a plot depicting expression of a target protein in vivo encoded by mRNA constructs having different stop codon elements: C1, C5, C10, C7, C8, and C9.

FIGS. 21B-21D are plots depicting expression of a target protein in vivo encoded by mRNA constructs having different stop codon elements: C1, C5, C10, C7, C8, and C9.

FIG. 21E is a plot depicting a time course of expression of a target protein in vivo encoded by mRNA constructs having different stop codon elements: C1, C5, C10, C7, C8, and C9.

FIG. 22A is a plot depicting expression of a target protein in vivo encoded by mRNA constructs having different stop codon elements: C1, C10, C7, C8, and C9.

FIG. 22B is a plot depicting a time course of expression of a target protein in vivo encoded by mRNA constructs having different stop codon elements: C1, C10, C7, and C8.

FIG. 22C is a plot depicting expression of a target protein in liver cells encoded by mRNA constructs having different stop codon elements: C1, C10, C7, C8, and C9.

FIG. 22D is a plot depicting expression of a target protein in spleen cells encoded by mRNA constructs having different stop codon elements: C1, C10, C7, C8, and C9.

FIG. 22E is a plot depicting expression of a target protein in vivo encoded by mRNA constructs having different stop codon elements: C1, C10, C7, C8, and C9.

FIGS. 23A-23D are plots depicting expression of a target protein in hepatocyte islands encoded by mRNA constructs having different stop codon elements: C1, C5, C10, C7, C8, and C9.

FIGS. 24A-24D are plots depicting expression of a target protein in hepatocyte islands encoded by mRNA constructs having different stop codon elements: C1, C5, C10, C7, C8, and C9.

FIGS. 25A-25C are plots depicting a time course of expression of a target protein in vivo in rat, cyno, and human hepatocyte islands. The target protein encoded by mRNA constructs having different stop codon elements: C1, C5, C10, C7, C8, and C9.

FIGS. 25D-25F are plots depicting a time course of expression of a target protein in vivo in rat, cyno, and human hepatocyte islands. The target protein encoded by mRNA constructs having different stop codon elements: C1, C5, C10, C7, C8, and C9.

FIGS. 26A-26B are plots depicting expression of an immune checkpoint protein in CD11c+MHCII+ cells at 24 and 72 hours after dosing with mRNA constructs with and without a 3′ stabilizing region.

FIGS. 27A-27C are plots depicting expression of an immune checkpoint protein in liver, spleen, and plasma of mice dosed with mRNA constructs with and without a 3′ stabilizing region.

FIGS. 28A-28B are plots depicting expression of an immune checkpoint protein in CD11c+MHCII+ cells at 72 and 120 hours after dosing with mRNA constructs with and without a 3′ stabilizing region.

FIGS. 29A-29D are plots depicting expression of an immune checkpoint protein in liver and spleen of mice 72 and 120 h after dosing with mRNA constructs with and without a 3′ stabilizing region.

FIGS. 30A-30C are plots depicting expression of an immune checkpoint protein in rat, cynomolgus and human hepatocytes. The target protein construct encoded by an mRNA with different stop codons: C1, C5, and C7, each with and without a 3′ stabilizing region.

FIGS. 31A-31C are plots depicting expression of an immune checkpoint protein in dendritic cells from individual donors. The target protein construct encoded by an mRNA with a 3′ stabilizing region, and different stop codons: C1, C5, and C7.

FIG. 32A is a plot depicting expression of a target protein in mice. The target protein construct encoded by an mRNA with different 5′ UTR and stop codon pairs: A11/C1, A1/C1, A11/C8, and A1/C8.

FIG. 32B is a plot depicting a time-course of expression of a target protein in mice. The target protein construct encoded by an mRNA with different 5′ UTR and stop codon pairs: A11/C1, A1/C1, A3/C1, A11/C8, A1/C8, and A3/C8.

FIGS. 32C-32D are plots depicting expression of a target protein in liver and spleen cells of mice. The target protein construct encoded by an mRNA with different 5′ UTR and stop codon pairs: A11/C1, A1/C1, A11/C8, and A1/C8.

FIG. 32E is a plot depicting expression of a target protein in mice. The target protein construct encoded by an mRNA with different 5′ UTR and stop codon pairs: A11/C1, A1/C1, A11/C8, and A1/C8.

FIG. 32F is a plot depicting a time-course of expression of a target protein in mice. The target protein construct encoded by an mRNA with different 5′ UTR and stop codon pairs: A11/C1, A1/C1, A3/C1, A11/C8, A1/C8, and A3/C8.

FIGS. 33A-33B are plots depicting the protein expression of a green fluorescent protein in HeLa cells. The target protein construct encoded by an mRNA with different 3′ UTRs: B10 and B18. Target protein levels were assessed over a time course of 60 hours.

FIGS. 33C-33D are plots depicting the protein expression of a green fluoresce protein in HeLa cells. The target protein construct encoded by an mRNA with different 3′ UTRs: B10 and B18. Target protein levels were assessed over a time course of 60 hours.

FIGS. 34A-34B are plots depicting the protein expression of target proteins in mice. The target protein construct encoded by an mRNA with different 3′ UTRs: B10 and B18. Target protein levels were assessed over a time course of 72 hours.

FIGS. 35A-35B are plots depicting expression of a target protein in mice over a time course of 120 hours. The target protein construct encoded by an mRNA with different 5′ UTRs: A12, A14, A15, A18, A20, A26, A27, and A11 (reference).

FIGS. 36A-36B are plots depicting expression of a target protein in mice 2 and 4 days after dosing with an mRNA construct, respectively. The target protein construct encoded by an mRNA with different 5′ UTRs: A12, A14, A20, A26, A27, A15, and A11.

FIGS. 36C-36D are plots depicting expression of a target protein in liver and spleen cells, respectively, harvested from mice 5 days after dosing with an mRNA construct. The target protein construct encoded by an mRNA with different 5′ UTRs: A12, A14, A20, A26, A27, A15, and A11.

### DETAILED DESCRIPTION

The potency and durability of mRNA can be optimized by: (1) ensuring that mRNAs delivered to the cytoplasm associate appropriately and productively with ribosomes; and (2) maximizing the time the mRNAs spend actively producing the desired protein product. The sequence of the mRNAs is an important determinant in performance across these aspects.

Disclosed herein, inter alia, is the discovery that the sequence for the 5′ untranslated region (UTR), 3′ UTR and/or stop element of an mRNA can be optimized to increase the potency and/or durability of said mRNA. In some embodiments, the disclosure provides polynucleotides and LNP compositions comprising optimized 5′ UTRs, 3′ UTRs and/or stop elements that can increase the efficacy, e.g., level and/or activity, of an mRNA or of a polypeptide encoded by the mRNA.

Exemplary effects on mRNA and/or encoded protein expression with mRNA constructs disclosed herein are provided in Examples 1-13 and 14-18. Examples 1-8 show increased level and/or activity of a target protein (e.g., increased protein expression, increased activity, and/or duration of protein expression) encoded by an mRNA having the A1 5′ UTR or variants thereof (A2 5′ UTR or A3 5′ UTR). Example 9 discloses the discovery and use of the B1 3′ UTR which extends the half-life of the mRNA construct. Examples 10-13 show increased level and/or activity of a target protein (e.g., increased protein expression, increased activity, and/or duration of protein expression) encoded by an mRNA having a stop element chosen from stop elements C2-C11. The in vivo effects of mRNA constructs having combinations of the 5′ UTR, 3′ UTR and/or stop elements disclosed herein is provided in Examples 15-18. The increased level and/or activity of a target protein is observed across cells types, species and target proteins.

Accordingly, disclosed herein are polynucleotides encoding a polypeptide, wherein the polynucleotide comprises: (a) a 5′-UTR (e.g., as described herein); (b) a coding region comprising a stop element (e.g., as described herein); and (c) a 3′-UTR (e.g., as described herein), and LNP compositions comprising the same. In an embodiment, the coding region comprises a polynucleotide sequence, e.g., mRNA, which encodes for a payload, e.g., a therapeutic payload or a prophylactic payload. In an embodiment, the polynucleotide, e.g., mRNA, or polypeptide encoded by the polynucleotide has an increased level and/or activity, e.g., expression or half-life. In an embodiment, the level and/or activity of the polynucleotide, e.g., mRNA, is increased. In an embodiment, the level and/or activity, or duration of expression of the polypeptide encoded by the polynucleotide is increased. Also disclosed herein are methods of using an LNP composition comprising a polynucleotide disclosed herein, for treating a disease or disorder, or for promoting a desired biological effect in a subject.

### Definitions

Polyuridine tract. A “polyuridine tract” or a “polyuracil tract” are used interchangeably herein and refer to a contiguous stretch of 2 or more uridines or uracils in a nucleic acid sequence. A polyuridine tract can be present at any position or section of a nucleic acid sequence. In an embodiment, a polyuridine tract is present in a 5′ UTR of a nucleic acid sequence. In an embodiment, a polyuridine tract comprises at least 2, 3, 4, 5, 6, 7, 8, 9, 10, 11, or 12 consecutive uridines. In an embodiment, a nucleic acid sequence can comprise more than 1 polyuridine tracts. In an embodiment, the more than one polyuridine tracts can be disposed adjacent to each other or separated by one or more nucleotides.

Uridine Content: The terms “uridine content” or “uracil content” are interchangeable and refer to the amount of uracil or uridine present in a certain nucleic acid sequence. Uridine content or uracil content can be expressed as an absolute value (total number of uridine or uracil in the sequence) or relative (uridine or uracil percentage respect to the total number of nucleobases in the nucleic acid sequence).

Stop element. A “stop element” as that term is used herein, refers to a nucleic acid sequence comprising a stop codon. The stop codon can be selected from TGA, TAA and TAG in the case of DNA, or from UGA, UAA and UAG in the case of RNA. In an embodiment, a stop element comprises two consecutive stop codons. In an embodiment, a stop element comprises three consecutive stop codons. In an embodiment, a stop element comprises four consecutive stop codons. In an embodiment, a stop element comprises five consecutive stop codons. In an embodiment, a stop element further comprises at least 1, 2, 3, 4, 5, 6, 7, 8, 9, 10, 11 or 12 nucleotides upstream and/or downstream of the one or more stop codons.

3′ stabilizing region. A “3′ stabilizing region” as that term is used herein, refers to a region that is made or becomes stable. A 3′ stabilizing region can be present at the 3′ terminus of a nucleic acids sequence. In an embodiment, a 3′ stabilizing region comprises a poly A tail, e.g., as described herein. In an embodiment, a 3′ stabilizing region comprises an alternative nucleoside, e.g., an inverted thymidine.

Sequence Identity: Calculations of sequence identity between sequences can be performed as follows. To determine the percent identity of two nucleotide sequences, the sequences are aligned for optimal comparison purposes (e.g., gaps can be introduced in one or both of a first and a second nucleotide sequence for optimal alignment). In some embodiments, the length of a reference sequence aligned for comparison purposes is at least 50%, e.g., at least 60%, 70%, 80%, 90%, or 100% of the length of the reference sequence. The nucleotides at corresponding nucleotide positions are compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. The percent identity typically refers to the ratio of the number of matching residues to the total length of the alignment. The comparison of sequences and determination of percent identity between two sequences can be accomplished using a mathematical algorithm. In some embodiments, the percent identity between two nucleotide sequences is determined using a pairwise sequence alignment program or a multiple sequence alignment program. Exemplary sequence alignment programs include, but are not limited to, the lalign program (embnet.vital-it.ch; Huang and Miller, (1991) Adv. Appl. Math. 12:337-357); the Clustal Omega program (www.ebi.ac.uk; Sievers et al. (2011) Mol. Syst. Biol. 7:539). In some embodiments, the default parameters of the program are used. The nucleotide sequences described herein can be used as a “query sequence” to perform a search against public databases to, for example, identify other family members or related sequences. Such searches can be performed using the BLAST® programs (blast.ncbi.nlm.nhi.gov; Altschul, et al. (1990) J. Mol. Biol. 215:403-10). For example, BLAST nucleotide searches can be performed with the blastn program to obtain nucleotide sequences identical or similar to a nucleotide sequence described herein. In some embodiments, the default parameters of the program are used.

Alternative nucleoside. An “alternative nucleoside” as that term is use herein, in reference to a nucleotide, nucleoside, or polynucleotide (such as the polynucleotides of the invention, e.g., mRNA molecule), refers to alteration with respect to A, G, U or C ribonucleotides. Generally, herein, these terms are not intended to refer to the ribonucleotide alterations in naturally occurring 5′-terminal mRNA cap moieties. The alterations may be various distinct alterations.

In some embodiments, where the polynucleotide is an mRNA, the coding region, the flanking regions and/or the terminal regions (e.g., a 3′-stabilizing region) may contain one, two, or more (optionally different) nucleoside or nucleotide alterations. In some embodiments, an alternative polynucleotide introduced to a cell may exhibit reduced degradation in the cell, as compared to an unaltered polynucleotide.

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. Preferred means of administration are intravenous or subcutaneous.

Antibody molecule: In one embodiment, antibody molecules can be used for targeting to desired cell types. As used herein, “antibody molecule” refers to a naturally occurring antibody, an engineered antibody, or a fragment thereof, e.g., an antigen binding portion of a naturally occurring antibody or an engineered antibody. An antibody molecule can include, e.g., an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). Exemplary antibody molecules include, but are not limited to, humanized antibody molecule, intact IgA, IgG, IgE or IgM antibody; bi- or multi-specific antibody (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of an LNP, “about” may mean +/−5% of the recited value. For instance, an LNP including a lipid component having about 40% of a given compound may include 30-50% of the compound.

Conjugated: As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, two or more moieties may be conjugated by direct covalent chemical bonding. In other embodiments, two or more moieties may be conjugated by ionic bonding or hydrogen bonding.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., a nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle) and a cell may be contacted, for example, by adding the composition to the culture medium of the cell and may involve or result in transfection. Moreover, more than one cell may be contacted by a nanoparticle composition.

Delivering: As used herein, the term “delivering” means providing an entity to a destination. For example, delivering a therapeutic and/or prophylactic to a subject may involve administering an LNP including the therapeutic and/or prophylactic to the subject (e.g., by an intravenous, intramuscular, intradermal, pulmonary or subcutaneous route). Administration of an LNP to a mammal or mammalian cell may involve contacting one or more cells with the lipid nanoparticle.

Encapsulate: As used herein, the term “encapsulate” means to enclose, surround, or encase. In some embodiments, a compound, polynucleotide (e.g., an mRNA), or other composition may be fully encapsulated, partially encapsulated, or substantially encapsulated. For example, in some embodiments, an mRNA of the disclosure may be encapsulated in a lipid nanoparticle, e.g., a liposome.

Encapsulation efficiency: As used herein, “encapsulation efficiency” refers to the amount of a therapeutic and/or prophylactic that becomes part of an LNP, relative to the initial total amount of therapeutic and/or prophylactic used in the preparation of an LNP. For example, if 97 mg of therapeutic and/or prophylactic are encapsulated in an LNP out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

Effective amount: As used herein, the term “effective amount” of an agent is that amount sufficient to effect beneficial or desired results, for example, clinical results, and, as such, an “effective amount” depends upon the context in which it is being applied. For example, in the context of the amount of a target cell delivery potentiating lipid in a lipid composition (e.g., LNP) of the disclosure, an effective amount of a target cell delivery potentiating lipid is an amount sufficient to effect a beneficial or desired result as compared to a lipid composition (e.g., LNP) lacking the target cell delivery potentiating lipid. Non-limiting examples of beneficial or desired results effected by the lipid composition (e.g., LNP) include increasing the percentage of cells transfected and/or increasing the level of expression of a protein encoded by a nucleic acid associated with/encapsulated by the lipid composition (e.g., LNP). In the context of administering a target cell delivery potentiating lipid-containing lipid nanoparticle such that an effective amount of lipid nanoparticles are taken up by target cells in a subject, an effective amount of target cell delivery potentiating lipid-containing LNP is an amount sufficient to effect a beneficial or desired result as compared to an LNP lacking the target cell delivery potentiating lipid. Non-limiting examples of beneficial or desired results in the subject include increasing the percentage of cells transfected, increasing the level of expression of a protein encoded by a nucleic acid associated with/encapsulated by the target cell delivery potentiating lipid-containing LNP and/or increasing a prophylactic or therapeutic effect in vivo of a nucleic acid, or its encoded protein, associated with/encapsulated by the target cell delivery potentiating lipid-containing LNP, as compared to an LNP lacking the target cell delivery potentiating lipid. In some embodiments, a therapeutically effective amount of target cell delivery potentiating lipid-containing LNP is sufficient, when administered to a subject suffering from or susceptible to an infection, disease, disorder, and/or condition, to treat, improve symptoms of, diagnose, prevent, and/or delay the onset of the infection, disease, disorder, and/or condition. In another embodiment, an effective amount of a lipid nanoparticle is sufficient to result in expression of a desired protein in at least about 5%, 10%, 15%, 20%, 25% or more of target cells. For example, an effective amount of target cell delivery potentiating lipid-containing LNP can be an amount that results in transfection of at least 5%, 10%, 15%, 20%, 25%, 30%, or 35% of target cells after a single intravenous injection.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Ex vivo: As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

Fragment: A “fragment,” as used herein, refers to a portion. For example, fragments of proteins may include polypeptides obtained by digesting full-length protein isolated from cultured cells or obtained through recombinant DNA techniques. A fragment of a protein can be, for example, a portion of a protein that includes one or more functional domains such that the fragment of the protein retains the functional activity of the protein.

GC-rich: As used herein, the term “GC-rich” refers to the nucleobase composition of a polynucleotide (e.g., mRNA), or any portion thereof (e.g., an RNA element), comprising guanine (G) and/or cytosine (C) nucleobases, or derivatives or analogs thereof, wherein the GC-content is greater than about 50%. The term “GC-rich” refers to all, or to a portion, of a polynucleotide, including, but not limited to, a gene, a non-coding region, a 5′ UTR, a 3′ UTR, an open reading frame, an RNA element, a sequence motif, or any discrete sequence, fragment, or segment thereof which comprises about 50% GC-content. In some embodiments of the disclosure, GC-rich polynucleotides, or any portions thereof, are exclusively comprised of guanine (G) and/or cytosine (C) nucleobases.

GC-content: As used herein, the term “GC-content” refers to the percentage of nucleobases in a polynucleotide (e.g., mRNA), or a portion thereof (e.g., an RNA element), that are either guanine (G) and cytosine (C) nucleobases, or derivatives or analogs thereof, (from a total number of possible nucleobases, including adenine (A) and thymine (T) or uracil (U), and derivatives or analogs thereof, in DNA and in RNA). The term “GC-content” refers to all, or to a portion, of a polynucleotide, including, but not limited to, a gene, a non-coding region, a 5′ or 3′ UTR, an open reading frame, an RNA element, a sequence motif, or any discrete sequence, fragment, or segment thereof.

Heterologous: As used herein, “heterologous” indicates that a sequence (e.g., an amino acid sequence or the polynucleotide that encodes an amino acid sequence) is not normally present in a given polypeptide or polynucleotide. For example, an amino acid sequence that corresponds to a domain or motif of one protein may be heterologous to a second protein. Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Kozak Sequence: The term “Kozak sequence” (also referred to as “Kozak consensus sequence”) refers to a translation initiation enhancer element to enhance expression of a gene or open reading frame, and which in eukaryotes, is located in the 5′ UTR. The Kozak consensus sequence was originally defined as the sequence GCCRCC (SEQ ID NO: 43), where R=a purine, following an analysis of the effects of single mutations surrounding the initiation codon (AUG) on translation of the preproinsulin gene (Kozak (1986) Cell 44:283-292). Polynucleotides disclosed herein comprise a Kozak consensus sequence, or a derivative or modification thereof. (Examples of translational enhancer compositions and methods of use thereof, see U.S. Pat. No. 5,807,707 to Andrews et al., incorporated herein by reference in its entirety; U.S. Pat. No. 5,723,332 to Chernajovsky, incorporated herein by reference in its entirety; U.S. Pat. No. 5,891,665 to Wilson, incorporated herein by reference in its entirety.)

Leaky scanning: A phenomenon known as “leaky scanning” can occur whereby the PIC bypasses the initiation codon and instead continues scanning downstream until an alternate or alternative initiation codon is recognized. Depending on the frequency of occurrence, the bypass of the initiation codon by the PIC can result in a decrease in translation efficiency. Furthermore, translation from this downstream AUG codon can occur, which will result in the production of an undesired, aberrant translation product that may not be capable of eliciting the desired therapeutic response. In some cases, the aberrant translation product may in fact cause a deleterious response (Kracht et al., (2017) Nat Med 23(4):501-507).

Liposome: As used herein, by “liposome” is meant a structure including a lipid-containing membrane enclosing an aqueous interior. Liposomes may have one or more lipid membranes. Liposomes include single-layered liposomes (also known in the art as unilamellar liposomes) and multi-layered liposomes (also known in the art as multilamellar liposomes).

Modified: As used herein “modified” refers to a changed state or structure of a molecule of the disclosure, e.g., a change in a composition or structure of a polynucleotide (e.g., mRNA). Molecules, e.g., polynucleotides, may be modified in various ways including chemically, structurally, and/or functionally. For example, molecules, e.g., polynucleotides, may be structurally modified by the incorporation of one or more RNA elements, wherein the RNA element comprises a sequence and/or an RNA secondary structure(s) that provides one or more functions (e.g., translational regulatory activity). Accordingly, molecules, e.g., polynucleotides, of the disclosure may be comprised of one or more modifications (e.g., may include one or more chemical, structural, or functional modifications, including any combination thereof). In one embodiment, polynucleotides, e.g., mRNA molecules, of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′ UTR, a 5′ cap and a polyA sequence. In an embodiment, the mRNA is a circular mRNA.

Nanoparticle: As used herein, “nanoparticle” refers to a particle having any one structural feature on a scale of less than about 1000 nm that exhibits novel properties as compared to a bulk sample of the same material. Routinely, nanoparticles have any one structural feature on a scale of less than about 500 nm, less than about 200 nm, or about 100 nm. Also routinely, nanoparticles have any one structural feature on a scale of from about 50 nm to about 500 nm, from about 50 nm to about 200 nm or from about 70 to about 120 nm. In exemplary embodiments, a nanoparticle is a particle having one or more dimensions of the order of about 1-1000 nm. In other exemplary embodiments, a nanoparticle is a particle having one or more dimensions of the order of about 10-500 nm. In other exemplary embodiments, a nanoparticle is a particle having one or more dimensions of the order of about 50-200 nm. A spherical nanoparticle would have a diameter, for example, of between about 50-100 or 70-120 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

Nucleic acid: As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Nucleic Acid Structure: As used herein, the term “nucleic acid structure” (used interchangeably with “polynucleotide structure”) refers to the arrangement or organization of atoms, chemical constituents, elements, motifs, and/or sequence of linked nucleotides, or derivatives or analogs thereof, that comprise a nucleic acid (e.g., an mRNA). The term also refers to the two-dimensional or three-dimensional state of a nucleic acid. Accordingly, the term “RNA structure” refers to the arrangement or organization of atoms, chemical constituents, elements, motifs, and/or sequence of linked nucleotides, or derivatives or analogs thereof, comprising an RNA molecule (e.g., an mRNA) and/or refers to a two-dimensional and/or three dimensional state of an RNA molecule. Nucleic acid structure can be further demarcated into four organizational categories referred to herein as “molecular structure”, “primary structure”, “secondary structure”, and “tertiary structure” based on increasing organizational complexity.

Nucleobase: As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines that confer improved properties (e.g., binding affinity, nuclease resistance, chemical stability) to a nucleic acid or a portion or segment thereof. Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids.

Nucleoside Nucleotide: As used herein, the term “nucleoside” refers to a compound containing a sugar molecule (e.g., a ribose in RNA or a deoxyribose in DNA), or derivative or analog thereof, covalently linked to a nucleobase (e.g., a purine or pyrimidine), or a derivative or analog thereof (also referred to herein as “nucleobase”), but lacking an internucleoside linking group (e.g., a phosphate group). As used herein, the term “nucleotide” refers to a nucleoside covalently bonded to an internucleoside linking group (e.g., a phosphate group), or any derivative, analog, or modification thereof that confers improved chemical and/or functional properties (e.g., binding affinity, nuclease resistance, chemical stability) to a nucleic acid or a portion or segment thereof.

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from an autoimmune disease, e.g., as described herein.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

RNA: As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (lncRNA) and mixtures thereof.

RNA element: As used herein, the term “RNA element” refers to a portion, fragment, or segment of an RNA molecule that provides a biological function and/or has biological activity (e.g., translational regulatory activity). Modification of a polynucleotide by the incorporation of one or more RNA elements, such as those described herein, provides one or more desirable functional properties to the modified polynucleotide. RNA elements, as described herein, can be naturally-occurring, non-naturally occurring, synthetic, engineered, or any combination thereof. For example, naturally-occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7(2):194-206), translation enhancer elements (e.g., the APP mRNA translation enhancer element, see Rogers et al., (1999) J Biol Chem 274(10):6421-6431), mRNA stability elements (e.g., AU-rich elements (AREs), see Garneau et al., (2007) Nat Rev Mol Cell Biol 8(2):113-126), translational repression element (see e.g., Blumer et al., (2002) Mech Dev 110(1-2):97-112), protein-binding RNA elements (e.g., iron-responsive element, see Selezneva et al., (2013) J Mol Biol 425(18):3301-3310), cytoplasmic polyadenylation elements (Villalba et al., (2011) Curr Opin Genet Dev 21(4):452-457), and catalytic RNA elements (e.g., ribozymes, see Scott et al., (2009) Biochim Biophys Acta 1789(9-10):634-641).

Specific delivery: As used herein, the term “specific delivery,” “specifically deliver,” or “specifically delivering” means delivery of more (e.g., at least 10% more, at least 20% more, at least 30% more, at least 40% more, at least 50% more, at least 1.5-fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target cell of interest (e.g., mammalian target cell) compared to an off-target cell (e.g., non-target cells). The level of delivery of a nanoparticle to a particular cell may be measured by comparing the amount of protein produced in target cells versus non-target cells (e.g., by mean fluorescence intensity using flow cytometry, comparing the % of target cells versus non-target cells expressing the protein (e.g., by quantitative flow cytometry), comparing the amount of protein produced in a target cell versus non-target cell to the amount of total protein in said target cells versus non-target cell, or comparing the amount of therapeutic and/or prophylactic in a target cell versus non-target cell to the amount of total therapeutic and/or prophylactic in said target cell versus non-target cell. It will be understood that the ability of a nanoparticle to specifically deliver to a target cell need not be determined in a subject being treated, it may be determined in a surrogate such as an animal model (e.g., a mouse or NHP model).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Targeting moiety: As used herein, a “targeting moiety” is a compound or agent that may target a nanoparticle to a particular cell, tissue, and/or organ type.

Therapeutic Agent: The term “therapeutic agent” refers to any agent that, when administered to a subject, has a therapeutic, diagnostic, and/or prophylactic effect and/or elicits a desired biological and/or pharmacological effect. In some embodiments, the therapeutic agent comprises or is a therapeutic payload. In some embodiments, the therapeutic agent comprises or is a small molecule or a biologic (e.g., an antibody molecule).

Transfection: As used herein, the term “transfection” refers to methods to introduce a species (e.g., a polynucleotide, such as a mRNA) into a cell.

Translational Regulatory Activity: As used herein, the term “translational regulatory activity” (used interchangeably with “translational regulatory function”) refers to a biological function, mechanism, or process that modulates (e.g., regulates, influences, controls, varies) the activity of the translational apparatus, including the activity of the PIC and/or ribosome. In some aspects, the desired translation regulatory activity promotes and/or enhances the translational fidelity of mRNA translation. In some aspects, the desired translational regulatory activity reduces and/or inhibits leaky scanning.

Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Uridine Content: The terms “uridine content” or “uracil content” are interchangeable and refer to the amount of uracil or uridine present in a certain nucleic acid sequence. Uridine content or uracil content can be expressed as an absolute value (total number of uridine or uracil in the sequence) or relative (uridine or uracil percentage respect to the total number of nucleobases in the nucleic acid sequence).

Uridine-Modified Sequence: The terms “uridine-modified sequence” refers to a sequence optimized nucleic acid (e.g., a synthetic mRNA sequence) with a different overall or local uridine content (higher or lower uridine content) or with different uridine patterns (e.g., gradient distribution or clustering) with respect to the uridine content and/or uridine patterns of a candidate nucleic acid sequence. In the content of the present disclosure, the terms “uridine-modified sequence” and “uracil-modified sequence” are considered equivalent and interchangeable.

A “high uridine codon” is defined as a codon comprising two or three uridines, a “low uridine codon” is defined as a codon comprising one uridine, and a “no uridine codon” is a codon without any uridines. In some embodiments, a uridine-modified sequence comprises substitutions of high uridine codons with low uridine codons, substitutions of high uridine codons with no uridine codons, substitutions of low uridine codons with high uridine codons, substitutions of low uridine codons with no uridine codons, substitution of no uridine codons with low uridine codons, substitutions of no uridine codons with high uridine codons, and combinations thereof. In some embodiments, a high uridine codon can be replaced with another high uridine codon. In some embodiments, a low uridine codon can be replaced with another low uridine codon. In some embodiments, a no uridine codon can be replaced with another no uridine codon. A uridine-modified sequence can be uridine enriched or uridine rarefied.

Uridine Enriched: As used herein, the terms “uridine enriched” and grammatical variants refer to the increase in uridine content (expressed in absolute value or as a percentage value) in a sequence optimized nucleic acid (e.g., a synthetic mRNA sequence) with respect to the uridine content of the corresponding candidate nucleic acid sequence. Uridine enrichment can be implemented by substituting codons in the candidate nucleic acid sequence with synonymous codons containing less uridine nucleobases. Uridine enrichment can be global (i.e., relative to the entire length of a candidate nucleic acid sequence) or local (i.e., relative to a subsequence or region of a candidate nucleic acid sequence).

Uridine Rarefied: As used herein, the terms “uridine rarefied” and grammatical variants refer to a decrease in uridine content (expressed in absolute value or as a percentage value) in an sequence optimized nucleic acid (e.g., a synthetic mRNA sequence) with respect to the uridine content of the corresponding candidate nucleic acid sequence. Uridine rarefication can be implemented by substituting codons in the candidate nucleic acid sequence with synonymous codons containing less uridine nucleobases. Uridine rarefication can be global (i.e., relative to the entire length of a candidate nucleic acid sequence) or local (i.e., relative to a subsequence or region of a candidate nucleic acid sequence).

Variant: As used herein, the term “variant” refers to a molecule having at least 50%, 60%, 70%, 80%, 85%, 90%, 95%, 96%, 97%, 98%, 99% or 100% activity of, or structural similarity to, the wild type molecule, e.g., as measured by an art-recognized assay.

### 5′ UTR Sequences

5′ UTR sequences are important for ribosome recruitment to the mRNA and have been reported to play a role in translation (Hinnebusch A, et al., (2016) Science, 352:6292: 1413-6).

Disclosed herein, inter alia, is a polynucleotide encoding a polypeptide, which polynucleotide has a 5′ UTR that confers an increased half-life, increased expression and/or increased activity of the polypeptide encoded by said polynucleotide, or of the polynucleotide itself. In an embodiment, a polynucleotide disclosed herein comprises: (a) a 5′-UTR (e.g., as provided in Table 1 or a variant or fragment thereof); (b) a coding region comprising a stop element (e.g., as described herein); and (c) a 3′-UTR (e.g., as described herein), and LNP compositions comprising the same. In an embodiment, the polynucleotide comprises a 5′-UTR comprising a sequence provided in Table 1 or a variant or fragment thereof (e.g., a functional variant or fragment thereof). It will be understood that such 5′ UTRs are incorporated into constructs not found in nature, e.g., such 5′ UTRs are synthetic, are altered in sequence from naturally occurring 5′ UTRs, are truncated or lengthened versions of those found in nature, comprise chemically modified bases, are 5′ of ORF sequences different from those which they may be found in nature, or the like.

In an embodiment, the fragment of a sequence provided in Table 1 lacks at least the first (i.e., 5′ most) one, two, three, four, five, or six nucleotides of the sequence provide in Table 1. In an embodiment, the fragment of a sequence provided in Table 1 lacks the first nucleotide of the sequence provide in Table 1. In an embodiment, the fragment of a sequence provided in Table 1 lacks the first two nucleotides of the sequence provide in Table 1. In an embodiment, the fragment of a sequence provided in Table 1 lacks the first three nucleotides of the sequence provide in Table 1. In an embodiment, the fragment of a sequence provided in Table 1 lacks the first four nucleotides of the sequence provide in Table 1. In an embodiment, the fragment of a sequence provided in Table 1 lacks the first five nucleotides of the sequence provide in Table 1. In an embodiment, the fragment of a sequence provided in Table 1 lacks the first six nucleotides of the sequence provide in Table 1.

In an embodiment, the polynucleotide comprises a 5′ UTR comprising a sequence that lacks at least the first one, two, three, four, five, or six nucleotides of a sequence provided in Table 1 but is otherwise at least 50% (e.g., at least 60%, 70%, 80%, 90%, 95%, 96%, 98%, 99%, or 100%) identical to the sequence provided in Table 1. In an embodiment, the polynucleotide comprises a 5′ UTR comprising a sequence that lacks the first nucleotide of a sequence provided in Table 1 but is otherwise at least 50% (e.g., at least 60%, 70%, 80%, 90%, 95%, 96%, 98%, 99%, or 100%) identical to the sequence provided in Table 1. In an embodiment, the polynucleotide comprises a 5′ UTR comprising a sequence that lacks the first two nucleotides of a sequence provided in Table 1 but is otherwise at least 50% (e.g., at least 60%, 70%, 80%, 90%, 95%, 96%, 98%, 99%, or 100%) identical to the sequence provided in Table 1. In an embodiment, the polynucleotide comprises a 5′ UTR comprising a sequence that lacks the first three nucleotides of a sequence provided in Table 1 but is otherwise at least 50% (e.g., at least 60%, 70%, 80%, 90%, 95%, 96%, 98%, 99%, or 100%) identical to the sequence provided in Table 1. In an embodiment, the polynucleotide comprises a 5′ UTR comprising a sequence that lacks the first four nucleotides of a sequence provided in Table 1 but is otherwise at least 50% (e.g., at least 60%, 70%, 80%, 90%, 95%, 96%, 98%, 99%, or 100%) identical to the sequence provided in Table 1. In an embodiment, the polynucleotide comprises a 5′ UTR comprising a sequence that lacks the first five nucleotides of a sequence provided in Table 1 but is otherwise at least 50% (e.g., at least 60%, 70%, 80%, 90%, 95%, 96%, 98%, 99%, or 100%) identical to the sequence provided in Table 1. In an embodiment, the polynucleotide comprises a 5′ UTR comprising a sequence that lacks the first six nucleotides of a sequence provided in Table 1 but is otherwise at least 50% (e.g., at least 60%, 70%, 80%, 90%, 95%, 96%, 98%, 99%, or 100%) identical to the sequence provided in Table 1.

In an embodiment, the polynucleotide having a 5′ UTR sequence provided in Table 1 or a variant or fragment thereof, has an increase in the half-life of the polynucleotide, e.g., about 1.5-20-fold increase in half-life of the polynucleotide. In an embodiment, the increase in half-life is about 1.5, 2, 3, 4, 5, 6, 7, 8, 9, 10, 11, 12, 13, 14, 15, 16, 17, 18, 19 or 20-fold, or more. In an embodiment, the increase in half life is about 1.5-fold or more. In an embodiment, the increase in half life is about 2-fold or more. In an embodiment, the increase in half life is about 3-fold or more. In an embodiment, the increase in half life is about 4-fold or more. In an embodiment, the increase in half life is about 5-fold or more.

In an embodiment, the polynucleotide having a 5′ UTR sequence provided in Table 1 or a variant or fragment thereof, results in an increased level and/or activity, e.g., output, of the polypeptide encoded by the polynucleotide. In an embodiment, the 5′ UTR results in about 1.5-20-fold increase in level and/or activity, e.g., output, of the polypeptide encoded by the polynucleotide. In an embodiment, the increase in level and/or activity is about 1.5, 2, 3, 4, 5, 6, 7, 8, 9, 10, 11, 12, 13, 14, 15, 16, 17, 18, 19 or 20-fold, or more. In an embodiment, the increase in level and/or activity is about 1.5-fold or more. In an embodiment, the increase in level and/or activity is about 2-fold or more. In an embodiment, the increase in level and/or activity is about 3-fold or more. In an embodiment, the increase in level and/or activity is about 4-fold or more. In an embodiment, the increase in level and/or activity is about 5-fold or more.

In an embodiment, the increase is compared to an otherwise similar polynucleotide which does not have a 5′ UTR, has a different 5′ UTR, or does not have a 5′ UTR described in Table 1 or a variant or fragment thereof.

In an embodiment, the increase in half-life of the polynucleotide is measured according to an assay that measures the half-life of a polynucleotide, e.g., an assay described in any one of Examples disclosed herein.

In an embodiment, the increase in level and/or activity, e.g., output, of the polypeptide encoded by the polynucleotide is measured according to an assay that measures the level and/or activity of a polypeptide, e.g., an assay described in any one of Examples disclosed herein.

In an embodiment, the 5′ UTR comprises a sequence provided in Table 1 or a sequence with at least 80%, 85%, 90%, 95%, 96%, 97%, 98%, 99% or 100% identity to a 5′ UTR sequence provided in Table 1, or a variant or a fragment thereof (e.g., a fragment that lacks the first one, two, three, four, five, or six nucleotides of the 5′ UTR sequence provided in Table 1). In an embodiment, the 5′ UTR comprises a sequence with at least 80%, 85%, 90%, 95%, 96%, 97%, 98%, 99% or 100% identity to SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 41, SEQ ID NO: 42, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 88, SEQ ID NO: 89 or SEQ ID NO: 90.

In an embodiment, the 5′ UTR comprises a sequence with at least 80%, 85%, 90%, 95%, 96%, 97%, 98%, 99% or 100% identity to a fragment of a 5′ UTR sequence provided in Table 1, e.g., SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 41, SEQ ID NO: 42, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 88, SEQ ID NO: 89 or SEQ ID NO: 90, wherein the fragment lacks at least the first one, two, three, four, five, or six nucleotides of the 5′ UTR sequence provided in Table 1, e.g., lacks nucleotide 1, nucleotides 1-2, nucleotides 1-3, nucleotides 1-4, or nucleotides 1-5 of any of SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 41, SEQ ID NO: 42, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 88, SEQ ID NO: 89 or SEQ ID NO: 90.

In an embodiment, the 5′ UTR comprises a sequence with at least 80%, 85%, 90%, 95%, 96%, 97%, 98%, 99% or 100% identity to SEQ ID NO: 1 or a fragment thereof that lacks the first one, two, three, four, five, six, or more nucleotides of SEQ ID NO: 1. In an embodiment, the 5′ UTR comprises a sequence with at least 80%, 85%, 90%, 95%, 96%, 97%, 98%, 99% or 100% identity to SEQ ID NO: 2 or a fragment thereof that lacks the first one, two, three, four, five, six, or more nucleotides of SEQ ID NO: 2. In an embodiment, the 5′ UTR comprises a sequence with at least 80%, 85%, 90%, 95%, 96%, 97%, 98%, 99% or 100% identity to SEQ ID NO: 3 or a fragment thereof that lacks the first one, two, three, four, five, six, or more nucleotides of SEQ ID NO: 3. In an embodiment, the 5′ UTR comprises a sequence with at least 80%, 85%, 90%, 95%, 96%, 97%, 98%, 99% or 100% identity to SEQ ID NO: 4 or a fragment thereof that lacks the first one, two, three, four, five, six, or more nucleotides of SEQ ID NO: 4. In an embodiment, the 5′ UTR comprises a sequence with at least 80%, 85%, 90%, 95%, 96%, 97%, 98%, 99% or 100% identity to SEQ ID NO: 5 or a fragment thereof that lacks the first one, two, three, four, five, six, or more nucleotides of SEQ ID NO: 5. In an embodiment, the 5′ UTR comprises a sequence with at least 80%, 85%, 90%, 95%, 96%, 97%, 98%, 99% or 100% identity to SEQ ID NO: 6 or a fragment thereof that lacks the first one, two, three, four, five, six, or more nucleotides of SEQ ID NO: 6. In an embodiment, the 5′ UTR comprises a sequence with at least 80%, 85%, 90%, 95%, 96%, 97%, 98%, 99% or 100% identity to SEQ ID NO: 8 or a fragment thereof that lacks the first one, two, three, four, five, six, or more nucleotides of SEQ ID NO: 8. In an embodiment, the 5′ UTR comprises a sequence with at least 80%, 85%, 90%, 95%, 96%, 97%, 98%, 99% or 100% identity to SEQ ID NO: 41 or a fragment thereof that lacks the first one, two, three, four, five, six, or more nucleotides of SEQ ID NO: 41. In an embodiment, the 5′ UTR comprises a sequence with at least 80%, 85%, 90%, 95%, 96%, 97%, 98%, 99% or 100% identity to SEQ ID NO: 42 or a fragment thereof that lacks the first one, two, three, four, five, six, or more nucleotides of SEQ ID NO: 42. In an embodiment, the 5′ UTR comprises a sequence with at least 80%, 85%, 90%, 95%, 96%, 97%, 98%, 99% or 100% identity to SEQ ID NO: 63 or a fragment thereof that lacks the first one, two, three, four, five, six, or more nucleotides of SEQ ID NO: 63. In an embodiment, the 5′ UTR comprises a sequence with at least 80%, 85%, 90%, 95%, 96%, 97%, 98%, 99% or 100% identity to SEQ ID NO: 64 or a fragment thereof that lacks the first one, two, three, four, five, six, or more nucleotides of SEQ ID NO: 64. In an embodiment, the 5′ UTR comprises a sequence with at least 80%, 85%, 90%, 95%, 96%, 97%, 98%, 99% or 100% identity to SEQ ID NO: 65 or a fragment thereof that lacks the first one, two, three, four, five, six, or more nucleotides of SEQ ID NO: 65. In an embodiment, the 5′ UTR comprises a sequence with at least 80%, 85%, 90%, 95%, 96%, 97%, 98%, 99% or 100% identity to SEQ ID NO: 66 or a fragment thereof that lacks the first one, two, three, four, five, six, or more nucleotides of SEQ ID NO: 66. In an embodiment, the 5′ UTR comprises a sequence with at least 80%, 85%, 90%, 95%, 96%, 97%, 98%, 99% or 100% identity to SEQ ID NO: 67 or a fragment thereof that lacks the first one, two, three, four, five, six, or more nucleotides of SEQ ID NO: 67. In an embodiment, the 5′ UTR comprises a sequence with at least 80%, 85%, 90%, 95%, 96%, 97%, 98%, 99% or 100% identity to SEQ ID NO: 68 or a fragment thereof that lacks the first one, two, three, four, five, six, or more nucleotides of SEQ ID NO: 68. In an embodiment, the 5′ UTR comprises a sequence with at least 80%, 85%, 90%, 95%, 96%, 97%, 98%, 99% or 100% identity to SEQ ID NO: 69 or a fragment thereof that lacks the first one, two, three, four, five, six, or more nucleotides of SEQ ID NO: 69. In an embodiment, the 5′ UTR comprises a sequence with at least 80%, 85%, 90%, 95%, 96%, 97%, 98%, 99% or 100% identity to SEQ ID NO: 70 or a fragment thereof that lacks the first one, two, three, four, five, six, or more nucleotides of SEQ ID NO: 70. In an embodiment, the 5′ UTR comprises a sequence with at least 80%, 85%, 90%, 95%, 96%, 97%, 98%, 99% or 100% identity to SEQ ID NO: 71 or a fragment thereof that lacks the first one, two, three, four, five, six, or more nucleotides of SEQ ID NO: 71. In an embodiment, the 5′ UTR comprises a sequence with at least 80%, 85%, 90%, 95%, 96%, 97%, 98%, 99% or 100% identity to SEQ ID NO: 72 or a fragment thereof that lacks the first one, two, three, four, five, six, or more nucleotides of SEQ ID NO: 72. In an embodiment, the 5′ UTR comprises a sequence with at least 80%, 85%, 90%, 95%, 96%, 97%, 98%, 99% or 100% identity to SEQ ID NO: 73 or a fragment thereof that lacks the first one, two, three, four, five, six, or more nucleotides of SEQ ID NO: 73. In an embodiment, the 5′ UTR comprises a sequence with at least 80%, 85%, 90%, 95%, 96%, 97%, 98%, 99% or 100% identity to SEQ ID NO: 74 or a fragment thereof that lacks the first one, two, three, four, five, six, or more nucleotides of SEQ ID NO: 74. In an embodiment, the 5′ UTR comprises a sequence with at least 80%, 85%, 90%, 95%, 96%, 97%, 98%, 99% or 100% identity to SEQ ID NO: 75 or a fragment thereof that lacks the first one, two, three, four, five, six, or more nucleotides of SEQ ID NO: 75. In an embodiment, the 5′ UTR comprises a sequence with at least 80%, 85%, 90%, 95%, 96%, 97%, 98%, 99% or 100% identity to SEQ ID NO: 76 or a fragment thereof that lacks the first one, two, three, four, five, six, or more nucleotides of SEQ ID NO: 76. In an embodiment, the 5′ UTR comprises a sequence with at least 80%, 85%, 90%, 95%, 96%, 97%, 98%, 99% or 100% identity to SEQ ID NO: 77 or a fragment thereof that lacks the first one, two, three, four, five, six, or more nucleotides of SEQ ID NO: 77. In an embodiment, the 5′ UTR comprises a sequence with at least 80%, 85%, 90%, 95%, 96%, 97%, 98%, 99% or 100% identity to SEQ ID NO: 78 or a fragment thereof that lacks the first one, two, three, four, five, six, or more nucleotides of SEQ ID NO: 78. In an embodiment, the 5′ UTR comprises a sequence with at least 80%, 85%, 90%, 95%, 96%, 97%, 98%, 99% or 100% identity to SEQ ID NO: 88 or a fragment thereof that lacks the first one, two, three, four, five, six, or more nucleotides of SEQ ID NO: 88. In an embodiment, the 5′ UTR comprises a sequence with at least 80%, 85%, 90%, 95%, 96%, 97%, 98%, 99% or 100% identity to SEQ ID NO: 89 or a fragment thereof that lacks the first one, two, three, four, five, six, or more nucleotides of SEQ ID NO: 89. In an embodiment, the 5′ UTR comprises a sequence with at least 80%, 85%, 90%, 95%, 96%, 97%, 98%, 99% or 100% identity to SEQ ID NO: 90 or a fragment thereof that lacks the first, two, three, four, five, six, or more nucleotides of SEQ ID NO: 90.

TABLE 1
5’ UTR sequences

| SEQ | | |
|---|---|---|
| ID | Sequence | |
| NO: | name | Sequence |
| 1 | A1 | GGAAAUCGCAAAAUUUGCUCUUCGCGUUAGAUUUCUUUUAGUUUUCUCG |
| | | CAACUAGCAAGCUUUUUGUUCUCGCC |
| 41 | A2 | GGAAAUCGCAAAAUUUGCUCUUCGCGUUAGAUUUCUUUUAGUUUUCUCG |
| | | CAACUAGCAAGCUUUUUGUUCUCGCCGCCGCC |
| 42 | A3 | GGAAAUCGCAAAAUUUUCUUUUCGCGUUAGAUUUCUUUUAGUUUUCUUU |
| | | CAACUAGCAAGCUUUUUGUUCUCGCCGCCGCC |
| 46 | A4 | G G A A A U C G C A A A A (N₂)ₓ (N₃)ₓ C U (N₄)ₓ (N₅)ₓ |
| | | C G C G U U A G A U U U C U U U U A G U U U U C U |
| | | N₆ N₇ C A A C U A G C A A G C U U U U U G U U C U C |
| | | G C C (N₈ C C)ₓ |
| | | (N₂)ₓ is a uracil and x is an integer from 0 to 5, e.g., wherein x = 3 or 4; |
| | | (N₃)ₓ is a guanine and x is an integer from 0 to 1; |
| | | (N₄)ₓ is a cytosine and x is an integer from 0 to 1; |
| | | (N₅)ₓ is a uracil and x is an integer from 0 to 5, e.g., wherein x = 2 or 3; |
| | | N₆ is a uracil or cytosine; |
| | | N₇ is a uracil or guanine; |
| | | N₈ is adenine or guanine and x is an integer from 0 to 1. |
| 2 | A5 | GGAAAUCCCCACAACCGCCUCAUAUCCAGGCUCAAGAAUAGAGCUCAGU |
| | | GUUUUGUUGUUUAAUCAUUCGGAGGUGUUUUGCGAUAUUCGCGCAAAGC |
| | | AGCCAGUCGCGCGCUUGCUUUUAAGUAGAGUUGUUUUUCCACCCGUUUG |
| | | CCAGGCAUCUUUAAUUUAACAUAUUUUUAUUUUUCAGGCUAACCUACGC |
| | | CGCCACC |
| 3 | A6 | GGAAAUAAGAGAGAAAAGAAGAGUAAGAAGAAAUAUAAGAUCUCCCUGA |
| | | GCUUCAGGGAGCCCCGGCGCCGCCACC |
| 4 | A7 | GGAAACCCCCCACCCCCGUAAGAGAGAAAAGAAGAGUAAGAAGAAAUAU |
| | | AAGAUCUCCCUGAGCUUCAGGGAGCCCCGGCGCCGCCACC |
| 5 | A8 | GGAGAACUUCCGCUUCCGUUGGCGCAAGCGCUUUCAUUUUUUCUGCUAC |
| | | CGUGACUAAG |
| 6 | A9 | GGAAAUAAGAGAGAAAAGAAGAGUAAGAAGAAAUAUAAGAGCCACC |
| 8 | A11 | GGAAAUAAGAGAGAAAAGAAGAGUAAGAAGAAAUAUAAGACCCCGGCGC |
| | (Reference) | CGCCACC |
| 63 | A12 | GGAAACUUUAUUUAGUGUUACUUUAUUUUCUGUUUAUUUGUGUUUCUUC |
| | | AGUGGGUUUGUUCUAAUUUCCUUGGCCGCC |
| 64 | A13 | GGAAAAUCUGUAUUAGGUUGGCGUGUUCUUUGGUCGGUUGUUAGUAUUG |
| | | UUGUUGAUUCGUUUGUGGUCGGUUGCCGCC |
| 65 | A14 | GGAAAAUUAUUAACAUCUUGGUAUUCUCGAUAACCAUUCGUUGGAUUUU |
| | | AUUGUAUUCGUAGUUUGGGUUCCUGCCGCC |
| 66 | A15 | GGAAAUUAUUAUUAUUUCUAGCUACAAUUUAUCAUUGUAUUAUUUUAGC |
| | | UAUUCAUCAUUAUUUACUUGGUGAUCAACA |
| 67 | A16 | GGAAAUAGGUUGUUAACCAAGUUCAAGCCUAAUAAGCUUGGAUUCUGGU |
| | | GACUUGCUUCACCGUUGGCGGGCACCGAUC |
| 68 | A17 | GGAAAUCGUAGAGAGUCGUACUUAGUACAUAUCGACUAUCGGUGGACAC |
| | | CAUCAAGAUUAUAAACCAGGCCAGA |
| 69 | A18 | GGAAACCCGCCCAAGCGACCCCAACAUAUCAGCAGUUGCCCAAUCCCAA |
| | | CUCCCAACACAAUCCCCAAGCAACGCCGCC |
| 70 | A19 | GGAAAGCGAUUGAAGGCGUCUUUUCAACUACUCGAUUAAGGUUGGGUAU |
| | | CGUCGUGGGACUUGGAAAUUUGUUGUUUCC |
| 71 | A20 | GGAAACUAAUCGAAAUAAAAGAGCCCCGUACUCUUUUAUUUCUAUUAGG |
| | | UUAGGAGCCUUAGCAUUUGUAUCUUAGGUA |
| 72 | A21 | GGAAAUGUGAUUUCCAGCAACUUCUUUUGAAUAUAUUGAAUUCCUAAUU |
| | | CAAAGCGAACAAAUCUACAAGCCAUAUACC |
| 73 | A22 | GGAAAUCGUAGAGAGUCGUACUUACGUGGUCGCCAUUGCAUAGCGCGCG |
| | | AAAGCAACAGGAACAAGAACGCGCC |
| 74 | A23 | GGAAAUCGUAGAGAGUCGUACUUAGAAUAAACAGAGUCGGGUCGACUUG |
| | | UCUCUGAUACUACGACGUCACAAUC |
| 75 | A24 | GGAAAAUUUGCCUUCGGAGUUGCGUAUCCUGAACUGCCCAGCCUCCUGA |
| | | UAUACAACUGUUCCGCUUAUUCGGGCCGCC |
| 76 | A25 | GGAAAUCUGAGCAGGAAUCCUUUGUGCAUUGAAGACUUUAGAUUCCUCU |
| | | CUGCGGUAGACGUGCACUUAUAAGUAUUUG |
| 77 | A26 | GGAAAGCGAUUGAAGGCGUCUUUUCAACUACUCGAUUAAGGUUGGGUAU |
| | | CGUCGUGGGACUUGGAAAUUUGUUGCCACC |
| 78 | A27 | GGAAAAUUUUAGCCUGGAACGUUAGAUAACUGUCCUGUUGUCUUUAUAU |
| | | ACUUGGUCCCCAAGUAGUUUGUCUUCCAAA |
| 88 | A28 | GGAAAUUUUUUUUUGAUAUUAUAAGAGUUUUUUUUUGAUAUUAAGAAAA |
| | | UUUUUUUUUGAUAUUAGAAGAGUAAGAAGAAAUAUAAGACCCCGGCGCC |
| | | GCCACC |
| 89 | A29 | GGAAAUAAGAGAGAAAAGAAGAGUAAGAAGAAAUAUAAGAGCCAAAAAA |
| | | AAAAAACC |
| 90 | A30 | GGAAAUCUCCCUGAGCUUCAGGGAGUAAGAGAGAAAAGAAGAGUAAGAA |
| | | GAAAUAUAAGACCCCGGCGCCGCCACC |
| 43 | A31 | GCCRCC, wherein R = A or G |

In an embodiment, the 5′ UTR comprises a variant of SEQ ID NO: 1. In an embodiment, the variant of SEQ ID NO: 1 comprises a nucleic acid sequence of Formula A: G G A A A U C G C A A A A (N₂)_{X} (N₃)_{X} C U (N₄)_{X} (N₅)_{X} C G C G U U A G A U U U C U U U U A G U U U U C U N₆ N₇ C A A C U A G C A A G C U U U U U G U U C U C G C C (N₈C C)_{X} (SEQ ID NO: 46),

wherein:

(N₂)ₓ is a uracil and x is an integer from 0 to 5, e.g., wherein x=3 or 4;

(N₃)ₓ is a guanine and x is an integer from 0 to 1;

(N₄)ₓ is a cytosine and x is an integer from 0 to 1;

(N₅)ₓ is a uracil and x is an integer from 0 to 5, e.g., wherein x=2 or 3;

N₆ is a uracil or cytosine;

N₇ is a uracil or guanine;

N₈ is adenine or guanine and x is an integer from 0 to 1.

In an embodiment (N₂)ₓ is a uracil and x is 0. In an embodiment (N₂)ₓ is a uracil and x is 1. In an embodiment (N₂)ₓ is a uracil and x is 2. In an embodiment (N₂)ₓ is a uracil and x is 3. In an embodiment, (N₂)ₓ is a uracil and x is 4. In an embodiment (N₂)ₓ is a uracil and x is 5.

In an embodiment, (N₃)ₓ is a guanine and x is 0. In an embodiment, (N₃)ₓ is a guanine and x is 1.

In an embodiment, (N₄)ₓ is a cytosine and x is 0. In an embodiment, (N₄)ₓ is a cytosine and x is 1.

In an embodiment (N₅)ₓ is a uracil and x is 0. In an embodiment (N₅)ₓ is a uracil and x is 1. In an embodiment (N₅)ₓ is a uracil and x is 2. In an embodiment (N₅)ₓ is a uracil and x is 3. In an embodiment, (N₅)ₓ is a uracil and x is 4. In an embodiment (N₅)ₓ is a uracil and x is 5.

In an embodiment, N6 is a uracil. In an embodiment, N6 is a cytosine.

In an embodiment, N7 is a uracil. In an embodiment, N7 is a guanine.

In an embodiment, N8 is an adenine and x is 0. In an embodiment, N8 is an adenine and x is 1.

In an embodiment, N8 is a guanine and x is 0. In an embodiment, N8 is a guanine and x is 1.

In an embodiment, the 5′ UTR comprises a variant of SEQ ID NO: 1. In an embodiment, the variant of SEQ ID NO: 1 comprises a sequence with at least 50%, 60%, 70%, 80%, 85%, 90%, 95%, 96%, 97%, 98%, or 99% identity to SEQ ID NO: 1 or a fragment thereof. In an embodiment, the variant of SEQ ID NO: 1 comprises a sequence with at least 50% identity to SEQ ID NO: 1 or a fragment thereof. In an embodiment, the variant of SEQ ID NO: 1 comprises a sequence with at least 60% identity to SEQ ID NO: 1 or a fragment thereof. In an embodiment, the variant of SEQ ID NO: 1 comprises a sequence with at least 70% identity to SEQ ID NO: 1 or a fragment thereof. In an embodiment, the variant of SEQ ID NO: 1 comprises a sequence with at least 80% identity to SEQ ID NO: 1 or a fragment thereof. In an embodiment, the variant of SEQ ID NO: 1 comprises a sequence with at least 90% identity to SEQ ID NO: 1 or a fragment thereof. In an embodiment, the variant of SEQ ID NO: 1 comprises a sequence with at least 95% identity to SEQ ID NO: 1 or a fragment thereof. In an embodiment, the variant of SEQ ID NO: 1 comprises a sequence with at least 96% identity to SEQ ID NO: 1 or a fragment thereof. In an embodiment, the variant of SEQ ID NO: 1 comprises a sequence with at least 97% identity to SEQ ID NO: 1 or a fragment thereof. In an embodiment, the variant of SEQ ID NO: 1 comprises a sequence with at least 98% identity to SEQ ID NO: 1 or a fragment thereof. In an embodiment, the variant of SEQ ID NO: 1 comprises a sequence with at least 99% identity to SEQ ID NO: 1 or a fragment thereof. In an embodiment, the fragment of SEQ ID NO: 1 comprises nucleotides 2-75 of SEQ ID NO: 1. In an embodiment, the fragment of SEQ ID NO: 1 comprises nucleotides 3-75 of SEQ ID NO: 1. In an embodiment, the fragment of SEQ ID NO: 1 comprises nucleotides 4-75 of SEQ ID NO: 1. In an embodiment, the fragment of SEQ ID NO: 1 comprises nucleotides 5-75 of SEQ ID NO: 1.

In an embodiment, the variant of SEQ ID NO: 1 comprises a uridine content of at least 5%, 10%, 20%, 30%, 40%, 50%, 60%, 70%, or 80%. In an embodiment, the variant of SEQ ID NO: 1 comprises a uridine content of at least 5%. In an embodiment, the variant of SEQ ID NO: 1 comprises a uridine content of at least 10%. In an embodiment, the variant of SEQ ID NO: 1 comprises a uridine content of at least 20%. In an embodiment, the variant of SEQ ID NO: 1 comprises a uridine content of at least 30%. In an embodiment, the variant of SEQ ID NO: 1 comprises a uridine content of at least 40%. In an embodiment, the variant of SEQ ID NO: 1 comprises a uridine content of at least 50%. In an embodiment, the variant of SEQ ID NO: 1 comprises a uridine content of at least 60%. In an embodiment, the variant of SEQ ID NO: 1 comprises a uridine content of at least 70%. In an embodiment, the variant of SEQ ID NO: 1 comprises a uridine content of at least 80%.

In an embodiment, the variant of SEQ ID NO: 1 comprises at least 2, 3, 4, 5, 6 or 7 consecutive uridines (e.g., a polyuridine tract). In an embodiment, the polyuridine tract in the variant of SEQ ID NO: 1 comprises at least 1-7, 2-7, 3-7, 4-7, 5-7, 6-7, 1-6, 1-5, 1-4, 1-3, 1-2, 2-6, or 3-5 consecutive uridines. In an embodiment, the polyuridine tract in the variant of SEQ ID NO: 1 comprises 4 consecutive uridines. In an embodiment, the polyuridine tract in the variant of SEQ ID NO: 1 comprises 5 consecutive uridines.

In an embodiment, the variant of SEQ ID NO: 1 comprises 1, 2, 3, 4, 5, 6, 7, 8, 9, 10, 11, 12, 13, 14, or 15 polyuridine tracts. In an embodiment, the variant of SEQ ID NO: 1 comprises 3 polyuridine tracts. In an embodiment, the variant of SEQ ID NO: 1 comprises 4 polyuridine tracts. In an embodiment, the variant of SEQ ID NO: 1 comprises 5 polyuridine tracts.

In an embodiment, one or more of the polyuridine tracts are adjacent to a different polyuridine tract. In an embodiment, each of, e.g., all, the polyuridine tracts are adjacent to each other, e.g., all of the polyuridine tracts are contiguous.

In an embodiment, one or more of the polyuridine tracts are separated by 1, 2, 3, 4, 5, 6, 7, 8, 9, 10, 11, 2, 13, 14, 15, 16, 17, 18. 19, 20, 30, 40, 50 or 60 nucleotides. In an embodiment, each of, e.g., all of, the polyuridine tracts are separated by 1, 2, 3, 4, 5, 6, 7, 8, 9, 10, 11, 2, 13, 14, 15, 16, 17, 18. 19, 20, 30, 40, 50 or 60 nucleotides.

In an embodiment, a first polyuridine tract and a second polyuridine tract are adjacent to each other.

In an embodiment, a subsequent, e.g., third, fourth, fifth, sixth or seventh, eighth, ninth, or tenth, polyuridine tract is separated by 1, 2, 3, 4, 5, 6, 7, 8, 9, 10, 11, 2, 13, 14, 15, 16, 17, 18. 19, 20, 30, 40, 50 or 60 nucleotides from the first polyuridine tract, the second polyuridine tract, or any one of the subsequent polyuridine tracts.

In an embodiment, a first polyuridine tract is separated by 1, 2, 3, 4, 5, 6, 7, 8, 9, 10, 11, 2, 13, 14, 15, 16, 17, 18. 19, 20, 30, 40, 50 or 60 nucleotides from a subsequent polyuridine tract, e.g., a second, third, fourth, fifth, sixth or seventh, eighth, ninth, or tenth polyuridine tract. In an embodiment, one or more of the subsequent polyuridine tracts are adjacent to a different polyuridine tract.

In an embodiment, the 5′ UTR comprises a Kozak sequence, e.g., a GCCRCC nucleotide sequence (SEQ ID NO: 43) wherein R is an adenine or guanine. In an embodiment, the Kozak sequence is disposed at the 3′ end of the 5′ UTR sequence.

In an embodiment, the polynucleotide comprising a 5′ UTR sequence disclosed herein comprises a coding region which encodes for a payload, e.g., a therapeutic or prophylactic payload.

In an aspect, the polynucleotide (e.g., mRNA) comprising a 5′ UTR sequence disclosed herein is formulated as an LNP. In an embodiment, the LNP composition comprises: (i) an ionizable lipid, e.g., an amino lipid; (ii) a sterol or other structural lipid; (iii) a non-cationic helper lipid or phospholipid; and (iv) a PEG-lipid.

In another aspect, the LNP compositions of the disclosure are used in a method of treating a disease or disorder, or in a method of inhibiting an immune response in a subject.

In an aspect, an LNP composition comprising a polynucleotide disclosed herein encoding a therapeutic payload or prophylactic payload, e.g., as described herein, can be administered with an additional agent, e.g., as described herein.

### 3′ UTR Sequences

3′ UTR sequences have been shown to influence translation, half-life, and subcellular localization of mRNAs (Mayr C., Cold Spring Harb Persp Biol 2019 Oct. 1; 11(10):a034728).

Disclosed herein, inter alia, is a polynucleotide encoding a polypeptide, which polynucleotide has a 3′ UTR that confers an increased half-life, increased expression and/or increased activity of the polypeptide encoded by said polynucleotide, or of the polynucleotide itself. In an embodiment, a polynucleotide disclosed herein comprises: (a) a 5′-UTR (e.g., as described herein); (b) a coding region comprising a stop element (e.g., as described herein); and (c) a 3′-UTR (e.g., as provided in Table 2 or a variant or fragment thereof), and LNP compositions comprising the same. In an embodiment, the polynucleotide comprises a 3′-UTR comprising a sequence provided in Table 2 or a variant or fragment thereof.

In an embodiment, the polynucleotide having a 3′ UTR sequence provided in Table 2 or a variant or fragment thereof, results in an increased half-life of the polynucleotide, e.g., about 1.5-10-fold increase in half-life of the polynucleotide. In an embodiment, the increase in half-life is about 1.5, 2, 3, 4, 5, 6, 7, 8, 9, or 10-fold, or more. In an embodiment, the increase in half-life is about 1.5-fold or more. In an embodiment, the increase in half-life is about 2-fold or more. In an embodiment, the increase in half-life is about 3-fold or more. In an embodiment, the increase in half-life is about 4-fold or more. In an embodiment, the increase in half-life is about 5-fold or more. In an embodiment, the increase in half-life is about 6-fold or more. In an embodiment, the increase in half-life is about 7-fold or more. In an embodiment, the increase in half-life is about 8-fold. In an embodiment, the increase in half-life is about 9-fold or more. In an embodiment, the increase in half-life is about 10-fold or more.

In an embodiment, the polynucleotide having a 3′ UTR sequence provided in Table 2 or a variant or fragment thereof, results in a polynucleotide with a mean half-life score of greater than 10.

In an embodiment, the polynucleotide having a 3′ UTR sequence provided in Table 2 or a variant or fragment thereof, results in an increased level and/or activity, e.g., output, of the polypeptide encoded by the polynucleotide.

In an embodiment, the increase is compared to an otherwise similar polynucleotide which does not have a 3′ UTR, has a different 3′ UTR, or does not have a 3′ UTR of Table 2 or a variant or fragment thereof.

In an embodiment, the polynucleotide comprises a 3′ UTR sequence provided in Table 2 or a sequence with at least 80%, 85%, 90%, 95%, 96%, 97%, 98%, 99% or 100% identity to a 3′ UTR sequence provided in Table 2, or a fragment thereof (e.g., a fragment that lacks the first (i.e., 5′ most) one, two, three, four, five, six, or more nucleotides of the 3′ UTR sequence provided in Table 2). In an embodiment, the 3′ UTR comprises a sequence with at least 80%, 85%, 90%, 95%, 96%, 97%, 98%, 99% or 100% identity to SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO:45, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 94 or SEQ ID NO: 95, or a fragment thereof (e.g., a fragment that lacks the first one, two, three, four, five, six, or more nucleotides of any of the aforesaid sequences). In an embodiment, the fragment lacks the first nucleotides of any of the aforesaid sequences. In an embodiment, the fragment lacks the first two nucleotides of any of the aforesaid sequences. In an embodiment, the fragment lacks the first three nucleotides of any of the aforesaid sequences. In an embodiment, the fragment lacks the first four nucleotides of any of the aforesaid sequences. In an embodiment, the fragment lacks the first five nucleotides of any of the aforesaid sequences. In an embodiment, the fragment lacks the first six nucleotides of any of the aforesaid sequences. In an embodiment, the fragment lacks the first seven or more (e.g., eight, nine, ten, or more) nucleotides of any of the aforesaid sequences.

In an embodiment, the 3′ UTR comprises a fragment of a 3′ UTR sequence provided in Table 2 such that the length of the combined stop element (e.g., a stop element described herein) and 3′ UTR has a constant length. For example, assuming a stop element that has X nucleotides is used in combination with a 3′ UTR sequence that has Y nucleotides, the combined length is X+Y nucleotides. In an embodiment, when a different stop element that has X+N nucleotides is used, the length of the 3′ UTR sequence will be shortened to Y−N nucleotides (e.g., by deleting the first N nucleotides of the 3′ UTR sequence), to keep the combined length constant (i.e., X+Y). In an embodiment, X=3, 6, 9, 12, or 15. In an embodiment, N==−15, −12, −9, −6, −3, 3, 6, 9, 12, or 15. In an embodiment, X=3, 6, 9, 12, or 15, and N=−15, −12, −9, −6, −3, 3, 6, 9, 12, or 15. In an embodiment, X=9 and N=6. In an embodiment, X=15 and N=−6. In an embodiment, the 3′ UTR comprises the sequence of SEQ ID NO: 11, or a sequence with at least 80%, 85%, 90%, 95%, 96%, 97%, 98%, 99% or 100% identity to SEQ ID NO: 11, or a fragment thereof (e.g., a fragment that lacks the first one, two, three, four, five, six, or more nucleotides of SEQ ID NO: 11). In an embodiment, the 3′ UTR comprises the sequence of SEQ ID NO: 12, or a sequence with at least 80%, 85%, 90%, 95%, 96%, 97%, 98%, 99% or 100% identity to SEQ ID NO: 12, or a fragment thereof (e.g., a fragment that lacks the first one, two, three, four, five, six, or more nucleotides of SEQ ID NO: 12). In an embodiment, the 3′ UTR comprises the sequence of SEQ ID NO: 13, or a sequence with at least 80%, 85%, 90%, 95%, 96%, 97%, 98%, 99% or 100% identity to SEQ ID NO: 13, or a fragment thereof (e.g., a fragment that lacks the first one, two, three, four, five, six, or more nucleotides of SEQ ID NO: 13). In an embodiment, the 3′ UTR comprises the sequence of SEQ ID NO: 14, or a sequence with at least 80%, 85%, 90%, 95%, 96%, 97%, 98%, 99% or 100% identity to SEQ ID NO: 14, or a fragment thereof (e.g., a fragment that lacks the first one, two, three, four, five, six, or more nucleotides of SEQ ID NO: 14). In an embodiment, the 3′ UTR comprises the sequence of SEQ ID NO: 15, or a sequence with at least 80%, 85%, 90%, 95%, 96%, 97%, 98%, 99% or 100% identity to SEQ ID NO: 15, or a fragment thereof (e.g., a fragment that lacks the first one, two, three, four, five, six, or more nucleotides of SEQ ID NO: 15). In an embodiment, the 3′ UTR comprises the sequence of SEQ ID NO: 16, or a sequence with at least 80%, 85%, 90%, 95%, 96%, 97%, 98%, 99% or 100% identity to SEQ ID NO: 16, or a fragment thereof (e.g., a fragment that lacks the first one, two, three, four, five, six, or more nucleotides of SEQ ID NO: 16). In an embodiment, the 3′ UTR comprises the sequence of SEQ ID NO: 17, or a sequence with at least 80%, 85%, 90%, 95%, 96%, 97%, 98%, 99% or 100% identity to SEQ ID NO: 17, or a fragment thereof (e.g., a fragment that lacks the first one, two, three, four, five, six, or more nucleotides of SEQ ID NO: 17). In an embodiment, the 3′ UTR comprises the sequence of SEQ ID NO: 18, or a sequence with at least 80%, 85%, 90%, 95%, 96%, 97%, 98%, 99% or 100% identity to SEQ ID NO: 18, or a fragment thereof (e.g., a fragment that lacks the first one, two, three, four, five, six, or more nucleotides of SEQ ID NO: 18). In an embodiment, the 3′ UTR comprises the sequence of SEQ ID NO: 19, or a sequence with at least 80%, 85%, 90%, 95%, 96%, 97%, 98%, 99% or 100% identity to SEQ ID NO: 19, or a fragment thereof (e.g., a fragment that lacks the first one, two, three, four, five, six, or more nucleotides of SEQ ID NO: 19). In an embodiment, the 3′ UTR comprises the sequence of SEQ ID NO: 20, or a sequence with at least 80%, 85%, 90%, 95%, 96%, 97%, 98%, 99% or 100% identity to SEQ ID NO: 20, or a fragment thereof (e.g., a fragment that lacks the first one, two, three, four, five, six, or more nucleotides of SEQ ID NO: 20). In an embodiment, the 3′ UTR comprises the sequence of SEQ ID NO: 21, or a sequence with at least 80%, 85%, 90%, 95%, 96%, 97%, 98%, 99% or 100% identity to SEQ ID NO: 21, or a fragment thereof (e.g., a fragment that lacks the first one, two, three, four, five, six, or more nucleotides of SEQ ID NO: 21). In an embodiment, the 3′ UTR comprises the sequence of SEQ ID NO: 22, or a sequence with at least 80%, 85%, 90%, 95%, 96%, 97%, 98%, 99% or 100% identity to SEQ ID NO: 22, or a fragment thereof (e.g., a fragment that lacks the first one, two, three, four, five, six, or more nucleotides of SEQ ID NO: 22). In an embodiment, the 3′ UTR comprises the sequence of SEQ ID NO: 23, or a sequence with at least 80%, 85%, 90%, 95%, 96%, 97%, 98%, 99% or 100% identity to SEQ ID NO: 23, or a fragment thereof (e.g., a fragment that lacks the first one, two, three, four, five, six, or more nucleotides of SEQ ID NO: 23). In an embodiment, the 3′ UTR comprises the sequence of SEQ ID NO: 24, or a sequence with at least 80%, 85%, 90%, 95%, 96%, 97%, 98%, 99% or 100% identity to SEQ ID NO: 24, or a fragment thereof (e.g., a fragment that lacks the first one, two, three, four, five, six, or more nucleotides of SEQ ID NO: 24). In an embodiment, the 3′ UTR comprises the sequence of SEQ ID NO: 25, or a sequence with at least 80%, 85%, 90%, 95%, 96%, 97%, 98%, 99% or 100% identity to SEQ ID NO: 25, or a fragment thereof (e.g., a fragment that lacks the first one, two, three, four, five, six, or more nucleotides of SEQ ID NO: 25). In an embodiment, the 3′ UTR comprises the sequence of SEQ ID NO: 45, or a sequence with at least 80%, 85%, 90%, 95%, 96%, 97%, 98%, 99% or 100% identity to SEQ ID NO: 45, or a fragment thereof (e.g., a fragment that lacks the first one, two, three, four, five, six, or more nucleotides of SEQ ID NO: 45). In an embodiment, the 3′ UTR comprises the sequence of SEQ ID NO: 79, or a sequence with at least 80%, 85%, 90%, 95%, 96%, 97%, 98%, 99% or 100% identity to SEQ ID NO: 79, or a fragment thereof (e.g., a fragment that lacks the first one, two, three, four, five, six, or more nucleotides of SEQ ID NO: 79). In an embodiment, the 3′ UTR comprises the sequence of SEQ ID NO: 80, or a sequence with at least 80%, 85%, 90%, 95%, 96%, 97%, 98%, 99% or 100% identity to SEQ ID NO: 80, or a fragment thereof (e.g., a fragment that lacks the first one, two, three, four, five, six, or more nucleotides of SEQ ID NO: 80). In an embodiment, the 3′ UTR comprises the sequence of SEQ ID NO: 81, or a sequence with at least 80%, 85%, 90%, 95%, 96%, 97%, 98%, 99% or 100% identity to SEQ ID NO: 81, or a fragment thereof (e.g., a fragment that lacks the first one, two, three, four, five, six, or more nucleotides of SEQ ID NO: 81). In an embodiment, the 3′ UTR comprises the sequence of SEQ ID NO: 82, or a sequence with at least 80%, 85%, 90%, 95%, 96%, 97%, 98%, 99% or 100% identity to SEQ ID NO: 82, or a fragment thereof (e.g., a fragment that lacks the first one, two, three, four, five, six, or more nucleotides of SEQ ID NO: 82). In an embodiment, the 3′ UTR comprises the sequence of SEQ ID NO: 83, or a sequence with at least 80%, 85%, 90%, 95%, 96%, 97%, 98%, 99% or 100% identity to SEQ ID NO: 83, or a fragment thereof (e.g., a fragment that lacks the first one, two, three, four, five, six, or more nucleotides of SEQ ID NO: 83). In an embodiment, the 3′ UTR comprises the sequence of SEQ ID NO: 84, or a sequence with at least 80%, 85%, 90%, 95%, 96%, 97%, 98%, 99% or 100% identity to SEQ ID NO: 84, or a fragment thereof (e.g., a fragment that lacks the first one, two, three, four, five, six, or more nucleotides of SEQ ID NO: 84). In an embodiment, the 3′ UTR comprises the sequence of SEQ ID NO: 85, or a sequence with at least 80%, 85%, 90%, 95%, 96%, 97%, 98%, 99% or 100% identity to SEQ ID NO: 85, or a fragment thereof (e.g., a fragment that lacks the first one, two, three, four, five, six, or more nucleotides of SEQ ID NO: 85). In an embodiment, the 3′ UTR comprises the sequence of SEQ ID NO: 86, or a sequence with at least 80%, 85%, 90%, 95%, 96%, 97%, 98%, 99% or 100% identity to SEQ ID NO: 86, or a fragment thereof (e.g., a fragment that lacks the first one, two, three, four, five, six, or more nucleotides of SEQ ID NO: 86). In an embodiment, the 3′ UTR comprises the sequence of SEQ ID NO: 87, or a sequence with at least 80%, 85%, 90%, 95%, 96%, 97%, 98%, 99% or 100% identity to SEQ ID NO: 87, or a fragment thereof (e.g., a fragment that lacks the first one, two, three, four, five, six, or more nucleotides of SEQ ID NO: 87).

In an embodiment, the 3′ UTR comprises the sequence of SEQ ID NO: 87, or a sequence with at least 80%, 85%, 90%, 95%, 96%, 97%, 98%, 99% or 100% identity to nucleotides 16-188 of SEQ ID NO: 60, or a fragment thereof (e.g., a fragment that lacks the first one, two, three, four, five, six, or more nucleotides of nucleotides 16-188 of SEQ ID NO: 60).

In an embodiment, the 3′ UTR comprises the sequence of SEQ ID NO: 87, or a sequence with at least 80%, 85%, 90%, 95%, 96%, 97%, 98%, 99% or 100% identity to nucleotides 16-188 of SEQ ID NO: 61, or a fragment thereof (e.g., a fragment that lacks the first one, two, three, four, five, six, or more nucleotides of nucleotides 16-188 of SEQ ID NO: 61). It will be understood that such 3′ UTRs are incorporated into constructs not found in nature, e.g., such 3′ UTRs are synthetic, are altered in sequence from naturally occurring 3′ UTRs, are truncated or lengthened versions of those found in nature, comprise chemically modified bases, are 3′ of ORF sequences different from those which they may be found in nature, or the like.

TABLE 2
3’ UTR sequences

| SEQ | | |
|---|---|---|
| ID | Sequence | |
| NO | information | Sequence |
| 1 | B1 | GCUGGAGCCUCCUGAGAGACCUGUGUGAACUAUUGAGAAGAUCGGAACA [symbol] |
| | | UCCUUACUCUGAGGAAGUUGGUACCCCCGUGGUCUUUGAAUAAAGUCUGA |
| | | UGGGCGGC |
| 45 | B2 | CUGAGAGACCUGUGUGAACUAUUGAGAAGAUCGGAACAGCUCCUUACUCU |
| | | AGGAAGUUG |
| 12 | B3 | GCUGGAGCCUCACUCUCCUCUCCAUCCCGUAUCCAGGCUGUGAAUUUUU [symbol] |
| | | AGGAAUAUAAAGAUCGGGAUGUACCCCCGUGGUCUUUGAAUAAAGUCUGA |
| | | UGGGCGGC |
| 13 | B4 | GCUGGAGCCUCUAGUGACGGCAACAGGGCUUGGUUUUUCCUUGUUGUGAA |
| | | UCGACAUCUCUGAAGACAGGGUACCCCCGUGGUCUUUGAAUAAAGUCUGA |
| | | UGGGCGGC |
| 14 | B5 | GCUGGAGCCUCCUUCCAUCUAGUCACAAAGACUCCUUCGUCCCCAGUUG [symbol] |
| | | GUCUAGGAUUGGGCCUCCCAGUACCCCCGUGGUCUUUGAAUAAAGUCUGA |
| | | UGGGCGGC |
| 15 | B6 | GCUGGAGCCUCCCAUAACAUGACAUAUCUGGAUUUUGUGCUUAGAACCUU |
| | | AAUUGGAAGCAUUCUUAAUUGUACCCCCGUGGUCUUUGAAUAAAGUCUGA |
| | | UGGGCGGC |
| 16 | B7 | GCUGGAGCCUCCGGAAAACUAAAAUAGAGAUAUUUCAAGAUUUUAUAAUU |
| | | UCAAAGACCUUUGAAAUAUUGUACCCCCGUGGUCUUUGAAUAAAGUCUGA |
| | | UGGGCGGC |
| 17 | B8 | GCUGGAGCCUCUACACAUUGCUUCUAGUUGGCAGAAAUAAUUGAUUAAAA |
| | | ACCAGAAACUGUGAUAACUGGUACCCCCGUGGUCUUUAAAUAAAGUCUAA |
| | | UGGGCGGC |
| 18 | B9 | GCUGGAGCCUCGGUGGCCUAGCUUCUUGCCCCUUGGGCCUCCCCCCAGC [symbol] |
| | | CUCCUCCCCUUCCUGCACCCGUACCCCCGUGGUCUUUGAAUAAAGUCAGA |
| | | UGGGCGGC |
| 19 | B10 | GCUGGAGUUUUGGUGGCCUAGCUUCUUGCCCUUUGGGCCUCCCCCCAGC [symbol] |
| | | CUCCUCCCCUUCCUGCACCCGUACCCCCGUGGUCUUUGAAUAAAGUCUGA |
| | | UGGGCGGC |
| 20 | B11 | GCUGGAGCCUCGGUGGCCAUGCUUCUUGCCCCUUGGGCCUCCCCCCAGC [symbol] |
| | | CUCCUCCCCUUCCUGCACCCGUACCCCCCAAACACCAUUGUCACACUCCA |
| | | UGGUCUUUGAAUAAAGUCUGAGUGGGCGGC |
| 21 | B12 | GCUGGAGCCUCGGUGGCCUAGCUUCUUGCCCCUUGGGCCUCCCCCCAGC [symbol] |
| | | CUCCUCCCCUUCCUGCACCCGUACCCCCCAAACACCAUUGUCACACUCCA |
| | | UGGUCUUUGAAUAAAGUCUGAGUGGGCGGC |
| 22 | B13 | GCUGGAGCCUCGGUGGCCUAGCUUCUUGCCCCUUGGGCCUCCCCCCAGC [symbol] |
| | | CUCCUCCCCUUCCUGCACCCGUACCCCCUCCAUAAAGUAGGAAACACUA [symbol] |
| | | GUGGUCUUUGAAUAAAGUCUGAGUGGGCGGC |
| 23 | B14 | GCUGGAGCCUCGGUGGCCUAGCUUCUUGCCCCUUGGGCCUCCCCCCAGC [symbol] |
| | | CUCCUCCCCUUCCUGCACCCGUACCCCCCGCAUUAUUACUCACGGUACGA |
| | | UGGUCUUUGAAUAAAGUCUGAGUGGGCGGC |
| 24 | B15 | UCCAUAAAGUAGGAAACACUACAGCUGGAGCCUCGGUGGCCAUGCUUCUU |
| | | CCCCUUGGGCCUCCCCCCAGCCCCUCCUCCCCUUCCUGCACCCGUACCC [symbol] |
| | | CGCAUUAUUACUCACGGUACGAGUGGUCUUUGAAUAAAGUCUGAGUGGG [symbol] |
| | | GC |
| 25 | B16 | UCCAUAAAGUAGGAAACACUACAGCUGGAGCCUCGGUGGCCUAGCUUCUU |
| | | CCCCUUGGGCCUCCAUAAAGUAGGAAACACUACAUCCCCCCAGCCCCUC [symbol] |
| | | CCCCUUCCUGCACCCGUACCCCCUCCAUAAAGUAGGAAACACUACAGUG [symbol] |
| | | CUUUGAAUAAAGUCUGAGUGGGCGGC |
| 79 | B17 | GCUGGAGCCUCUCACACACCUCUGCCCCUUGGGCCUCCCACUCCCAUGG [symbol] |
| | | CUGGGCGGUCCAGAAGGAGCGUACCCCCGUGGUCUUUGAAUAAAGUCUGA |
| | | UGGGCGGC |
| 80 | B18 | GCUGGAGCCUCCACCGCGUUAUCCGUUCCUCGUAGGCUGGUCCUGGGGAA |
| | | GGGUCGGCGGGUACCCCCGUGGUCUUUGAAUAAAGUCUGAGUGGGCGGC |
| 81 | B19 | GCUGGAGCCUCUGCCCGGCAACGGCCAGGUCUGUGCCAAGUGUUUGCUGA |
| | | GCAACCCCCACUGGCUGGGGCUUGGUCAUGGGCCAUCAGCGCGUGCGUG [symbol] |
| | | ACCUUUUCGGCUCCUCUGCCGAUCCAUACUGCGGAACUCCUAGCCGCUU [symbol] |
| | | UUUGCUCGCAGCAGGUCUGGAGCAAACAUUAUCGGGACUGAUAACUCUGU |
| | | GUCCUGUACCCCCGUGGUCUUUGAAUAAAGUCUGAGUGGGCGGC |
| 82 | B20 | GCUGGAGCCUCGGUGGCCUAGCUUCUUGCCCCUUGGGCCUCCCCCCAGC [symbol] |
| | | CUCCUCCCCUUCCUGCACCCGUACCCUUUUUUUUUUUUUUUUUUUCUUCU |
| | | UUCUUUUUUUUCUUUUUUUUUUUUCUUUCUUUUUUUCUUUUUUUUUCUUU |
| | | CUUUUUUCUUUUUUUUUUUUUUUUCCGUGGUCUUUGAAUAAAGUCUGAGU |
| | | GGCGGC |
| 83 | B21 | GCUGGAGCCUCGGUGGCCUAGCUUCUUGCCCCUUGGGCCUCCCCCCAGC [symbol] |
| | | CUCCUCCCCUUCCUGCACCCGUACCCUUUUUUUUUUUUUUUUUUUUUUUU |
| | | UUUUUUUUUUUUUUUUUUUUUUUUUUUUUUUUUUUUUUUUUUUUUUUUUU |
| | | CUUUUUUCUUUUUUUUUUUUUUUUCCGUGGUCUUUGAAUAAAGUCUGAGU |
| | | GGCGGC |
| 84 | B22 | AUAAAGUAGGAAACACUACAGCUGGAGCCUCGGUGGCCUAGCUUCUUGC [symbol] |
| | | CUUGGGCCUCCAUAAAGUAGGAAACACUACAUCCCCCCAGCCCCUCCUC [symbol] |
| | | CUUCCUGCACCCGUACCCCCUCCAUAAAGUAGGAAACACUACAGUGGUCU |
| | | UGAAUAAAGUCUGAGUGGGCGGC |
| 85 | B23 | GGAGCCUCGGUGGCCUAGCUUCUUGCCCCUUGGGCCUCCCCCCAGCCCCU |
| | | CUCCCCUUCCUGCACCCGUACCCCCUCCAUAAAGUAGGAAACACUACAGU |
| | | GUCUUUGAAUAAAGUCUGAGUGGGCGGC |
| 86 | B24 | UCCAUAAAGUAGGAAACACUACAGCUGGAGCCUCCUGAGAGACCUGUGU [symbol] |
| | | ACUAUUGAGAAGAUCGGAACAGCUCCUUACUCUGAGGAAGUUGUCCAUAA |
| | | GUAGGAAACACUACAGUACCCCCUCCAUAAAGUAGGAAACACUACAGUG [symbol] |
| | | CUUUGAAUAAAGUCUGAGUGGGCGGC |
| 87 | B25 | ACCUCACUCACGGCCACAUUGAGUGCCAGGCUCCGGGCUGGUUUAUAGUA |
| | | UGUAGAGCAUUGCAGCACUUAGACUGGGGUGCUGUAGUCUUUAUUGUAGU |
| | | UUUCCACAUACCUGAUAAUUCUUAGAUAAUUUCUUAUUUUAAUUCCAUAA |
| | | GUAGGAAACACUACAUAAAUCUCCAUAAAGUAGGAAACACUACAUAUUCU |
| | | CCAUAAAGUAGGAAACACUACAUAGGCU |
| 94 | B26 | GCCUCCACCGCGUUAUCCGUUCCUCGUAGGCUGGUCCUGGGGAACGGGU [symbol] |
| | | GCGGGUACCCCCGUGGUCUUUGAAUAAAGUCUGAGUGGGCGGC |
| 95 | B27 | CACCGCGUUAUCCGUUCCUCGUAGGCUGGUCCUGGGGAACGGGUCGGCG [symbol] |
| | | CCUCGGUGGCCUAGCUUCUUGCCCCUUGGGCCCACCGCGUUAUCCGUUC [symbol] |
| | | CGUAGGCUGGUCCUGGGGAACGGGUCGGCGGUCCCCCCAGCCCCUCCUC [symbol] |
| | | CUUCCUGCACCCGUACCCCCCACCGCGUUAUCCGUUCCUCGUAGGCUGG [symbol] |
| | | CUGGGGAACGGGUCGGCGGGUGGUCUUUGAAUAAAGUCUGAGUGGGCGG [symbol] |

[symbol]

indicates data missing or illegible when filed

In an embodiment, the 3′ UTR comprises a micro RNA (miRNA) binding site, e.g., as described herein, which binds to a miR present in a human cell. In an embodiment, the 3′ UTR comprises a miRNA binding site of SEQ ID NO: 38, SEQ ID NO: 39, SEQ ID NO: 40 or a combination thereof. In an embodiment, the 3′ UTR comprises a plurality of miRNA binding sites, e.g., 2, 3, 4, 5, 6, 7 or 8 miRNA binding sites. In an embodiment, the plurality of miRNA binding sites comprises the same or different miRNA binding sites.

miR122 bs =
(SEQ ID NO: 38)
CAAACACCAUUGUCACACUCCA
miR-142-3p bs =
(SEQ ID NO: 39)
UCCAUAAAGUAGGAAACACUACA
miR-126 bs =
(SEQ ID NO: 40)
CGCAUUAUUACUCACGGUACGA

In an embodiment, the 3′ UTR comprises a TENT recruiting sequence, e.g., as described herein, which recruits one or more terminal nucleotidyl transferases (TENTs) to the polynucleotide comprising the 3′ UTR. In an embodiment, the TENT is TENT4, e.g., TENT4A and/or TENT4B. Without wishing to be bound by theory, it is believed that in some embodiments one or more TENTs (e.g., TENT4A and/or TENT4B) generates a mixed poly-A tail with intermittent non-adenosine residues (e.g., guanosine), which shields mRNA from rapid deadenylation.

Exemplary TENT recruiting sequences include, but are not limited to,

(SEQ ID NO: 91)
CACCGCGUUAUCCGUUCCUCGUAGGCUGGUCCUGGGGAACGGGUCGGCGG
and
(SEQ ID NO: 92)
CCACCCCCAGCGCCACCACCGCUGCCGUCGCCACCGCGUUAUCCGUUCCU
CGUAGGCUGGUCCUGGGGAACGGGUCGGCGGCCGGUCGGCUUCUGUUUUA

In an embodiment, the TENT recruiting sequence comprises the nucleotide sequence of SEQ ID NO: 91, or a nucleotide sequence having at least 80%, 85%, 90%, 95%, 96%, 97%, 98%, or 99% identity thereto, or differing by no more than 1, 2, 3, 4, 5, 6, 7, 8, 9, or 10 nucleotides therefrom. In an embodiment, the TENT recruiting sequence comprises the nucleotide sequence of SEQ ID NO: 91.

In an embodiment, the TENT recruiting sequence comprises the nucleotide sequence of SEQ ID NO: 92, or a nucleotide sequence having at least 80%, 85%, 90%, 95%, 96%, 97%, 98%, or 99% identity thereto, or differing by no more than 1, 2, 3, 4, 5, 6, 7, 8, 9, 10, 11, 12, 13, 14, 15, 16, 17, 18, 19, or 20 nucleotides therefrom. In an embodiment, the TENT recruiting sequence comprises the nucleotide sequence of SEQ ID NO: 92.

In an embodiment, the 3′ UTR comprises one or more (e.g., 2, 3, 4, 5, or more) TENT recruiting sequences, e.g., one or more TENT recruiting sequences described herein. In an embodiment the 3′ UTR comprises one TENT recruiting sequence. In an embodiment the 3′ UTR comprises two TENT recruiting sequences. In an embodiment the 3′ UTR comprises three TENT recruiting sequences. In an embodiment the 3′ UTR comprises four TENT recruiting sequences. In an embodiment the 3′ UTR comprises five TENT recruiting sequences. For example, the multiple TENT recruiting sequences in the 3′ UTR can be identical or different.

In an embodiment, the 3′ UTR comprises a TENT recruiting sequence comprising the nucleotide sequence of SEQ ID NO: 91, or a nucleotide sequence having at least 80%, 85%, 90%, 95%, 96%, 97%, 98%, or 99% identity thereto, or differing by no more than 1, 2, 3, 4, 5, 6, 7, 8, 9, or 10 nucleotides therefrom. In an embodiment, the 3′ UTR comprises a TENT recruiting sequence comprising the nucleotide sequence of SEQ ID NO: 91.

In an embodiment, the 3′ UTR comprises one or more (e.g., 2, 3, 4, 5, or more) of a TENT recruiting sequence comprising the nucleotide sequence of SEQ ID NO: 91, or a nucleotide sequence having at least 80%, 85%, 90%, 95%, 96%, 97%, 98%, or 99% identity thereto, or differing by no more than 1, 2, 3, 4, 5, 6, 7, 8, 9, or 10 nucleotides therefrom. In an embodiment, the 3′ UTR comprises one TENT recruiting sequence comprising the nucleotide sequence of SEQ ID NO: 91, or a nucleotide sequence having at least 80%, 85%, 90%, 95%, 96%, 97%, 98%, or 99% identity thereto, or differing by no more than 1, 2, 3, 4, 5, 6, 7, 8, 9, or 10 nucleotides therefrom. In an embodiment, the 3′ UTR comprises two TENT recruiting sequences, each comprising the nucleotide sequence of SEQ ID NO: 91, or a nucleotide sequence having at least 80%, 85%, 90%, 95%, 96%, 97%, 98%, or 99% identity thereto, or differing by no more than 1, 2, 3, 4, 5, 6, 7, 8, 9, or 10 nucleotides therefrom. In an embodiment, the 3′ UTR comprises three TENT recruiting sequences, each comprising the nucleotide sequence of SEQ ID NO: 91, or a nucleotide sequence having at least 80%, 85%, 90%, 95%, 96%, 97%, 98%, or 99% identity thereto, or differing by no more than 1, 2, 3, 4, 5, 6, 7, 8, 9, or 10 nucleotides therefrom. In an embodiment, the 3′ UTR comprises four TENT recruiting sequences, each comprising the nucleotide sequence of SEQ ID NO: 91, or a nucleotide sequence having at least 80%, 85%, 90%, 95%, 96%, 97%, 98%, or 99% identity thereto, or differing by no more than 1, 2, 3, 4, 5, 6, 7, 8, 9, or 10 nucleotides therefrom. In an embodiment, the 3′ UTR comprises five TENT recruiting sequences, each comprising the nucleotide sequence of SEQ ID NO: 91, or a nucleotide sequence having at least 80%, 85%, 90%, 95%, 96%, 97%, 98%, or 99% identity thereto, or differing by no more than 1, 2, 3, 4, 5, 6, 7, 8, 9, or 10 nucleotides therefrom.

In an embodiment, the 3′ UTR comprises one or more (e.g., 2, 3, 4, 5, or more) of a TENT recruiting sequence comprising the nucleotide sequence of SEQ ID NO: 91. In an embodiment, the 3′ UTR comprises two TENT recruiting sequences, each comprising the nucleotide sequence of SEQ ID NO: 91. In an embodiment, the 3′ UTR comprises three TENT recruiting sequences, each comprising the nucleotide sequence of SEQ ID NO: 91. In an embodiment, the 3′ UTR comprises four TENT recruiting sequences, each comprising the nucleotide sequence of SEQ ID NO: 91. In an embodiment, the 3′ UTR comprises five TENT recruiting sequences, each comprising the nucleotide sequence of SEQ ID NO: 91.

In an embodiment, the 3′ UTR comprises the nucleotide sequence of SEQ ID NO: 80, or a nucleotide sequence having at least 80%, 85%, 90%, 95%, 96%, 97%, 98%, or 99% identity thereto, or differing by no more than 1, 2, 3, 4, 5, 6, 7, 8, 9, 10, 11, 12, 13, 14, 15, 16, 17, 18, 19, or 20 nucleotides therefrom. In an embodiment, the 3′ UTR comprises the nucleotide sequence of SEQ ID NO: 80.

In an embodiment, the 3′ UTR comprises the nucleotide sequence of SEQ ID NO: 94, or a nucleotide sequence having at least 80%, 85%, 90%, 95%, 96%, 97%, 98%, or 99% identity thereto, or differing by no more than 1, 2, 3, 4, 5, 6, 7, 8, 9, 10, 11, 12, 13, 14, 15, 16, 17, 18, 19, or 20 nucleotides therefrom. In an embodiment, the 3′ UTR comprises the nucleotide sequence of SEQ ID NO: 94.

In an embodiment, the 3′ UTR comprises the nucleotide sequence of SEQ ID NO: 95, or a nucleotide sequence having at least 80%, 85%, 90%, 95%, 96%, 97%, 98%, or 99% identity thereto, or differing by no more than 1, 2, 3, 4, 5, 6, 7, 8, 9, 10, 11, 12, 13, 14, 15, 16, 17, 18, 19, or 20 nucleotides therefrom. In an embodiment, the 3′ UTR comprises the nucleotide sequence of SEQ ID NO: 95.

In an aspect, disclosed herein is a polynucleotide encoding a polypeptide, wherein the polynucleotide comprises: (a) a 5′-UTR, e.g., as described herein; (b) a coding region comprising a stop element (e.g., as described herein); and (c) a 3′-UTR (e.g., as described herein).

In an aspect, an LNP composition comprising a polynucleotide comprising a 3′ UTR disclosed herein comprises: (i) an ionizable lipid, e.g., an amino lipid; (ii) a sterol or other structural lipid; (iii) a non-cationic helper lipid or phospholipid; and (iv) a PEG-lipid.

In another aspect, the LNP compositions of the disclosure are used in a method of treating a disease or disorder, or in a method of inhibiting an immune response in a subject.

In an aspect, an LNP composition comprising a polynucleotide disclosed herein encoding a therapeutic payload or prophylactic payload, e.g., as described herein, can be administered with an additional agent, e.g., as described herein.

### Stop Element

Translational stop codons, UAA, UAG, and UGA, are an important component of the genetic code and signal the termination of translation of an mRNA. During protein synthesis, stop codons interact with protein release factors and this interaction can modulate ribosomal activity thus having an impact translation (Tate W P, et al., (2018) Biochem Soc Trans, 46(6):1615-162).

Disclosed herein, inter alia, is a polynucleotide encoding a polypeptide, which polynucleotide has a coding region comprising a stop element which confers an increased half-life, increased expression and/or increased activity of the polypeptide encoded by said polynucleotide, or of the polynucleotide itself. In an embodiment, the polynucleotide comprises: (a) a 5′-UTR (e.g., as described herein); (b) a coding region comprising a stop element (e.g., as described herein); and (c) a 3′-UTR (e.g., as described herein), and LNP compositions comprising the same. In an embodiment, the polynucleotide comprises a coding region comprising a stop element provided in Table 3.

A stop element as used herein, refers to a nucleic acid sequence comprising a stop codon. The stop codon can be selected from TGA, TAA and TAG in the case of DNA, or from UGA, UAA and UAG in the case of RNA. In an embodiment, a stop element comprises two consecutive stop codons. In an embodiment, a stop element comprises three consecutive stop codons. In an embodiment, a stop element comprises four consecutive stop codons. In an embodiment, a stop element comprises five consecutive stop codons.

In an embodiment, the stop element comprises a plurality of the same stop codon. In an embodiment, the stop element comprises a plurality of different stop codons.

In an embodiment, a stop element further comprises at least 1, 2, 3, 4, 5, or 10 nucleotides upstream and/or downstream of the one or more stop codons. In an embodiment, a stop element further comprises at least 1, 2, 3, 4, 5, or 10 nucleotides upstream of the one or more stop codons. In an embodiment, a stop element further comprises at least 1, 2, 3, 4, 5, or 10 nucleotides downstream of the one or more stop codons.

The invention also includes a polynucleotide that comprises both a stop codon element and the polynucleotide described herein. In some embodiments, a stop codon element comprises a stop codon region. In some embodiments, the coding region of the polynucleotide comprises the stop element. In some embodiments, the stop element is upstream, e.g., before, the 3′ UTR sequence in the polynucleotide.

In some embodiments, the polynucleotides of the present invention can include at least two stop codons before the 3′ untranslated region (UTR). The stop codon can be selected from TGA, TAA and TAG in the case of DNA, or from UGA, UAA and UAG in the case of RNA. In some embodiments, the polynucleotides of the present invention include the stop codon TGA in the case or DNA, or the stop codon UGA in the case of RNA, and one additional stop codon. In a further embodiment the addition stop codon can be TAA or UAA. In another embodiment, the polynucleotides of the present invention include three consecutive stop codons, four stop codons, or more.

It has been observed that stop elements comprising a sequence provided in Table 3 can result in increased half-life of the polynucleotide and/or increased level or activity of the polypeptide encoded by the polynucleotide.

In an embodiment, the polynucleotide having a stop element provided in Table 3 results in an increased half-life of the polynucleotide or an increased level and/or activity, e.g., output, of the polypeptide encoded by the polynucleotide. In an embodiment, the increase in half-life is about 1.5-20-fold. In an embodiment, the increase in half-life is about 1.5, 2, 3, 4, 5, 6, 7, 8, 9, 10, 11, 12, 13, 14, 15, 16, 17, 18, 19 or 20-fold, or more. In an embodiment, the increase in half life is about 1.5-fold or more. In an embodiment, the increase in half life is about 2-fold or more. In an embodiment, the increase in half life is about 3-fold or more. In an embodiment, the increase in half life is about 4-fold. In an embodiment, the increase in half life is about 5-fold or more.

In an embodiment, the polynucleotide having a stop element provided in Table 3 results in an increased level and/or activity, e.g., output or duration of expression, of the polypeptide encoded by the polynucleotide. In an embodiment, the stop element results in about 1.5-20-fold increase in level and/or activity, e.g., detectable level or activity, of the polypeptide encoded by the polynucleotide for about 1, 2, 3, 4, 5, 6, 7, 8, 9, 10, or 14 days. In an embodiment, the stop element results in detectable level or activity of the polypeptide encoded by the polynucleotide for about 1, 2, 3, 4, 5, 6, 7, 8, 9, 10, or 14 days.

In an embodiment, the increase in activity is about 1.5, 2, 3, 4, 5, 6, 7, 8, 9, 10, 11, 12, 13, 14, 15, 16, 17, 18, 19 or 20-fold, or more. In an embodiment, the increase in activity is about 1.5-fold or more. In an embodiment, the increase in activity is about 2-fold or more. In an embodiment, the increase in activity is about 3-fold or more. In an embodiment, the increase in activity is about 4-fold or more. In an embodiment, the increase in activity is about 5-fold or more.

In an embodiment, the increase is compared to an otherwise similar polynucleotide which does not have a stop element, has a different stop element, or does not have a stop element provided in Table 3.

In an embodiment, the stop element comprises a sequence provided in Table 3. In an embodiment, the stop element comprises the sequence of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 or SEQ ID NO:36, SEQ ID NO; 62, SEQ ID NO: 93 or SEQ ID NO: 96. In an embodiment, the stop element comprises the sequence of SEQ ID NO: 26. In an embodiment, the stop element comprises the sequence of SEQ ID NO: 27. In an embodiment, the stop element comprises the sequence of SEQ ID NO: 28. In an embodiment, the stop element comprises the sequence of SEQ ID NO: 29. In an embodiment, the stop element comprises the sequence of SEQ ID NO: 30 In an embodiment, the stop element comprises the sequence of SEQ ID NO: 31. In an embodiment, the stop element comprises the sequence of SEQ ID NO: 32. In an embodiment, the stop element comprises the sequence of SEQ ID NO: 33. In an embodiment, the stop element comprises the sequence of SEQ ID NO: 34. In an embodiment, the stop element comprises the sequence of SEQ ID NO: 35. In an embodiment, the stop element comprises the sequence of SEQ ID NO: 36. In an embodiment, the stop element comprises the sequence of SEQ ID NO: 62. In an embodiment, the stop element comprises the sequence of SEQ ID NO: 93. In an embodiment, the stop element comprises the sequence of SEQ ID NO; 96.

In an embodiment, the coding region of (b) comprises a stop element comprising a consensus sequence of Formula B:

(SEQ ID NO: 37)
X₋₃-X₋₂-X₋₁-U-A-A-X₁-X₂-X₃-X₄-X₅-X₆-X₇-X₈-X₉-X₁₀-X₁₁-X₁₂

wherein:

X₁ is a G or A;

X₂, X₄, X₅ X₆ or X₇ is each independently C or U;

X₃ is C or A;

X₈, X₁₀, X₁₁, X₁₂ X₋₁ or X₋₃ is each independently C or G;

X₉ is G or U; and/or X₋₂ is A or U.

In an embodiment, X₁ is a G. In an embodiment, X₁ is an A.

In an embodiment, X₂ is a C. In an embodiment, X₂ is a U.

In an embodiment, X₄ is a C. In an embodiment, X₄ is a U.

In an embodiment, X₅ is a C. In an embodiment, X₅ is a U.

In an embodiment, X₆ is a C. In an embodiment, X₆ is a U.

In an embodiment, X₇ is a C. In an embodiment, X₇ is a U.

In an embodiment, X₃ is a C. In an embodiment, X₃ is an A.

In an embodiment, X₈ is a C. In an embodiment, X₈ is a G.

In an embodiment, X₁₀ is a C. In an embodiment, X₁₀ is a G.

In an embodiment, X₁₁ is a C. In an embodiment, X₁₁ is a G.

In an embodiment, X₁₂ is a C. In an embodiment, X₁₂ is a G.

In an embodiment, X₋₁ is a C. In an embodiment, X₋₁ is a G.

In an embodiment, X₋₃ is a C. In an embodiment, X₋₃ is a G.

In an embodiment, X₉ is a G. In an embodiment, X₉ is a U.

In an embodiment, X₋₂ is an A. In an embodiment, X₋₂ is a U.

In an embodiment, the consensus sequence of Formula B (SEQ ID NO: 37) has a high GC content, e.g., GC content of about 50%, 60%, 70%, 80%, 90% or 99%. In an embodiment, the GC content is about 50%. In an embodiment, the GC content is about 60%. In an embodiment, the GC content is about 70%. In an embodiment, the GC content is about 80%. In an embodiment, the GC content is about 90%. In an embodiment, the GC content is about 99%.

In an embodiment, the coding region of (b) comprises a stop element comprising a consensus sequence of Formula C:

(SEQ ID NO: 56)
X₋₃-X₋₂-X₋₁-U-G-A-X₁-X₂-X₃-X₄-X₅-X₆-X₇-X₈-X₉-X₁₀-X₁₁-X₁₂

wherein:

X₋₃, X₋₁, X₂, X₅, X₆, X₇, X₈, X₉, or X₁₂ is each independently G or C;

X₋₂, X₃, or X₄ is each independent A or C;

X₁ is A or G; and/or

X₁₀ or X₁₁ is each independently C or U.

In an embodiment, X₋₃ is a G. In an embodiment, X₋₃ is a C.

In an embodiment, X₋₁ is a G. In an embodiment, X₋₁ is a C.

In an embodiment, X₂ is a G. In an embodiment, X₂ is a C.

In an embodiment, X₅ is a G. In an embodiment, X₅ is a C.

In an embodiment, X₆ is a G. In an embodiment, X₆ is a C.

In an embodiment, X₇ is a G. In an embodiment, X₇ is a C.

In an embodiment, X₈ is a G. In an embodiment, X₈ is a C.

In an embodiment, X₉ is a G. In an embodiment, X₉ is a C.

In an embodiment, X₁₂ is a G. In an embodiment, X₁₂ is a C.

In an embodiment, X₋₂ is an A. In an embodiment, X₋₂ is a C.

In an embodiment, X₃ is an A. In an embodiment, X₃ is a C.

In an embodiment, X₄ is an A. In an embodiment, X₄ is a C.

In an embodiment, X₁ is an A. In an embodiment, X₁ is a G.

In an embodiment, X₁₀ is a C. In an embodiment, X₁₀ is a U.

In an embodiment, X₁₁ is a C. In an embodiment, X₁₁ is a U.

In an embodiment, the consensus sequence of Formula C (SEQ ID NO: 56) has a high GC content, e.g., GC content of about 50%, 60%, 70%, 80%, 90% or 99%. In an embodiment, the GC content is about 50%. In an embodiment, the GC content is about 60%. In an embodiment, the GC content is about 70%. In an embodiment, the GC content is about 80%. In an embodiment, the GC content is about 90%. In an embodiment, the GC content is about 99%.

In an embodiment, the coding region of (b) comprises a stop element comprising a consensus sequence of Formula D:

(SEQ ID NO: 57)
X₋₃-X₋₂-X₋₁-U-A-G-X₁-X₂-X₃-X₄-X₅-X₆-X₇-X₈-X₉-X₁₀-X₁₁-X₁₂

wherein:

X₋₃, X₋₁, X₂, X₃, X₁₀ is each independently G or C;

X₋₂ or X₉ is each independently A or U;

X₁ or X₄ is each independently A or G;

X₅ or X₈ is each independently A or C; and/or

X₆, X₇, X₁₁ or X₁₂ is each independently C or U.

In an embodiment, X₋₃ is a G. In an embodiment, X₋₃ is a C.

In an embodiment, X₋₁ is a G. In an embodiment, X₋₁ is a C.

In an embodiment, X₂ is a G. In an embodiment, X₂ is a C.

In an embodiment, X₃ is a G. In an embodiment, X₃ is a C.

In an embodiment, X₁₀ is a G. In an embodiment, X₁₀ is a C.

In an embodiment, X₋₂ is an A. In an embodiment, X₋₂ is a U.

In an embodiment, X₉ is an A. In an embodiment, X₉ is a U.

In an embodiment, X₁ is an A. In an embodiment, X₁ is a G.

In an embodiment, X₄ is an A. In an embodiment, X₄ is a G.

In an embodiment, X₅ is an A. In an embodiment, X₅ is a C.

In an embodiment, X₈ is an A. In an embodiment, X₈ is a C.

In an embodiment, X₆ is a C. In an embodiment, X₆ is a U.

In an embodiment, X₇ is a C. In an embodiment, X₇ is a U.

In an embodiment, X₁₁ is a C. In an embodiment, X₁₁ is a U.

In an embodiment, X₁₂ is a C. In an embodiment, X₁₂ is a U.

In an embodiment, the consensus sequence of Formula D (SEQ TD NO: 57) has a high GC content, e.g., GC content of about 50%, 60%, 70%, 80%, 90% or 99%. In an embodiment, the GC content is about 5000. In an embodiment, the GC content is about 60%. In an embodiment, the GC content is about 70%. In an embodiment, the GC content is about 80%. In an embodiment, the GC content is about 90%. In an embodiment, the GC content is about 9900.

TABLE 3
Stop elements

| SEQ ID | Sequence | |
|---|---|---|
| NO | information | Sequence |
| 26 | C1 | UGAUAAUAG |
| | (reference) | |
| 27 | C2 | UAAUAGUAA |
| 28 | C3 | UAAGUCUAA |
| 29 | C4 | UAAAGCUAA |
| 30 | C5 | UAAGUCUCC |
| 31 | C6 | UAAGGCUAA |
| 32 | C7 | UAAGCCCCUCCGGGG |
| 33 | C8 | UAAAGCUCCCCGGGG |
| 34 | C9 | UAAGCCCCU |
| 35 | C10 | UAAAGCUCC |
| 36 | C11 | UAGGGUUAA |
| 62 | C15 | UAAGCACCC |
| 37 | C12(UAA | X₋₃-X₋₂-X₋₁-U-A-A-X₁-X₂-X₃-X₄-X₅-X₆-X₇−X₈-X₉- |
| | consensus) | X₁₀-X₁₁-X₁₂ |
| | | wherein: |
| | | X₁ is a G or A; |
| | | X₂, X₄, X₅ X₆ or X₇ is each independently C or U; |
| | | X₃ is C or A; |
| | | X₈, X₁₀, X₁₁, X₁₂ X₋₁ or X₋₃ is each independently C |
| | | or G; |
| | | X₉ is G or U; and/or |
| | | X₋₂ is A or U. |
| 56 | C13 (UGA | X₋₃-X₋₂-X₋₁-U-G-A-X₁-X₂-X₃-X₄-X₅-X₆-X₇−X₈-X₉- |
| | consensus) | X₁₀-X₁₁-X₁₂ |
| | | wherein: |
| | | X₋₃, X₋₁, x₂, X₃, X₄, X₇, X₈, X₉, or X₁₂ is each |
| | | independently G or C; |
| | | X₋₂, X₃, or X₄ is each independent A or C; |
| | | X₁ is A or G; and/or |
| | | X₁₀ or X₁₁ is each independently C or U. |
| 57 | C14(UAG | X₋₃-X₋₂-X₋₁-U-A-G-X₁-X₂-X₃-X₄-X₅-X₆-X₇-X₈-X₉- |
| | consensus) | X₁₀-X₁₁-X₁₂ |
| | | wherein: |
| | | X₋₃, X₋₁, X₂, X₃, X₁₀ is each independently G or C; |
| | | X₋₂ or X₉ is each independently A or U; |
| | | X₁ or X₄ is each independently A or G; |
| | | X₅ or X₈ is each independently A or C; and/or |
| | | X₆, X₇, X₁₁ or X₁₂ is each independently C or U. |
| 93 | C16 | UGAUAGUAA |
| 96 | C17 | UAAAGCGCU |

In an aspect, disclosed herein is a polynucleotide encoding a polypeptide, wherein the polynucleotide comprises: (a) a 5′-UTR, e.g., as described herein; (b) a coding region comprising a stop element (e.g., as provided in Table 3); and (c) a 3′-UTR (e.g., as described herein).

In an aspect, an LNP composition comprising a polynucleotide comprising a stop element disclosed herein comprises: (i) an ionizable lipid, e.g., an amino lipid; (ii) a sterol or other structural lipid; (iii) a non-cationic helper lipid or phospholipid; and (iv) a PEG-lipid.

In another aspect, the LNP compositions of the disclosure are used in a method of treating a disease or disorder, or in a method of inhibiting an immune response in a subject.

In an aspect, an LNP composition comprising a polynucleotide disclosed herein encoding a therapeutic payload or prophylactic payload, e.g., as described herein, can be administered with an additional agent, e.g., as described herein.

### 3′ Stabilizing Region

Disclosed herein, inter alia, is a polynucleotide encoding a polypeptide, wherein the polynucleotide comprises: (a) a 5′-UTR (e.g., as described herein); (b) a coding region comprising a stop element (e.g., as described herein); (c) a 3′-UTR (e.g., as described herein), and (d) a 3′ stabilizing region. Also disclosed herein are LNP compositions comprising the same.

In an embodiment, the polynucleotide comprises a 3′ stabilizing region, e.g., a stabilized tail e.g., as described herein. A polynucleotide containing a 3′-stabilizing region (e.g., a 3′-stabilizing region including an alternative nucleobase, sugar, and/or backbone) may be particularly effective for use in therapeutic compositions, because they may benefit from increased stability, high expression levels. An exemplary method of making a polynucleotide having a 3′ stabilized region is described in Example 14.

In an embodiment, the 3′ stabilizing region comprises a poly A tail, e.g., a poly A tail comprising 80-150, e.g., 120, adenines (SEQ ID NO: 123). In an embodiment, the poly A tail comprises one or more non-adenosine residues, e.g., one or more guanosines, e.g., as described herein. In an embodiment, the poly A tail comprises a UCUAG sequence (SEQ ID NO: 44). In an embodiment, the poly A tail comprises about 80-120, e.g., 100, adenines upstream of SEQ ID NO: 44. In an embodiment, the poly A tail comprises about 1-40, e.g., 20, adenines downstream of SEQ ID NO: 44.

In an embodiment, the 3′ stabilizing region comprises at least one alternative nucleoside. In an embodiment, the alternative nucleoside is an inverted thymidine (idT). In an embodiment, the alternative nucleoside is disposed at the 3′ end of the 3′ stabilizing region.

In an embodiment, the 3′ stabilizing region comprises a structure of Formula VII:

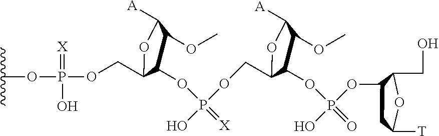

or a salt thereof, wherein each X is independently O or S, and A represents adenine and T represents thymine.

In an aspect, disclosed herein is a polynucleotide encoding a polypeptide, wherein the polynucleotide comprises: (a) a 5′-UTR, e.g., as described herein; (b) a coding region comprising a stop element (e.g., as described herein); (c) a 3′-UTR (e.g., as described herein) and; (d) a 3′ stabilizing region, e.g., as described herein.

In an aspect, an LNP composition comprising a polynucleotide comprising a stabilizing region disclosed herein comprises: (i) an ionizable lipid, e.g., an amino lipid; (ii) a sterol or other structural lipid; (iii) a non-cationic helper lipid or phospholipid; and (iv) a PEG-lipid.

In another aspect, the LNP compositions of the disclosure are used in a method of treating a disease or disorder, or in a method of inhibiting an immune response in a subject.

In an aspect, an LNP composition comprising a polynucleotide disclosed herein encoding a therapeutic payload or prophylactic payload, e.g., as described herein, can be administered with an additional agent, e.g., as described herein.

Constructs Comprising mRNA Elements

It will be understood that the regulatory elements disclosed herein (e.g., 5′ UTRs, stop elements, 3′ UTRs, stabilizing regions (e.g., idT or modified poly A tails) can be used with ORFs encoding any peptide or protein of interest, e.g., therapeutic or prophylactic proteins, whether, e.g., intracellular, transmembrane, or secreted. It will further be understood that the regulatory elements disclosed herein can be used in a modular fashion, i.e., can be used in an mRNA construct in combination with other regulatory elements from the art (e.g., a 5′ UTR of the instant invention in combination with an ORF and other regulatory regions from the art), or can be used in combination with the other regulatory elements disclosed herein (e.g., a 5′ UTR of the instant invention and a 3′ UTR of the instant invention, et cetera). It will further be understood that a stop element of the present invention can be used in combination with a desired ORF that lacks a stop codon. It will also be understood that when a desired ORF comprises a stop codon, an additional stop codon or stop element will not be included in the final construct. In some embodiments, the stop codon in the desired ORF can be replaced with a stop element described herein.

Combination of mRNA Elements

Any of the polynucleotides disclosed herein can comprise one, two, three, or all of the following elements: (a) a 5′-UTR, e.g., as described herein; (b) a coding region comprising a stop element (e.g., as described herein); (c) a 3′-UTR (e.g., as described herein) and; optionally (d) a 3′ stabilizing region, e.g., as described herein. Also disclosed herein are LNP compositions comprising the same.

In an embodiment, a polynucleotide of the disclosure comprises (a) a 5′ UTR described in Table 1 or a variant or fragment thereof and (b) a coding region comprising a stop element provided in Table 3. In an embodiment, the polynucleotide further comprises a cap structure, e.g., as described herein, or a poly A tail, e.g., as described herein. In an embodiment, the polynucleotide further comprises a 3′ stabilizing region, e.g., as described herein.

In an embodiment, the polynucleotide comprises (a) a 5′ UTR comprising the sequence of SEQ ID NO: 8 or a variant or fragment thereof, and (b) a coding region comprising a stop element comprising the sequence of SEQ ID NO: 26.

In an embodiment, the polynucleotide comprises (a) a 5′ UTR comprising the sequence of SEQ ID NO: 1 or a variant or fragment thereof, and (b) a coding region comprising a stop element comprising the sequence of SEQ ID NO: 26.

In an embodiment, the polynucleotide comprises (a) a 5′ UTR comprising the sequence of SEQ ID NO: 42 or a variant or fragment thereof, and (b) a coding region comprising a stop element comprising the sequence of SEQ ID NO: 26.

In an embodiment, the polynucleotide comprises (a) a 5′ UTR comprising the sequence of SEQ ID NO: 8 or a variant or fragment thereof, and (b) a coding region comprising a stop element comprising the sequence of SEQ ID NO: 33.

In an embodiment, the polynucleotide comprises (a) a 5′ UTR comprising the sequence of SEQ ID NO: 1 or a variant or fragment thereof, and (b) a coding region comprising a stop element comprising the sequence of SEQ ID NO: 33.

In an embodiment, the polynucleotide comprises (a) a 5′ UTR comprising the sequence of SEQ ID NO: 42 or a variant or fragment thereof, and (b) a coding region comprising a stop element comprising the sequence of SEQ ID NO: 33.

In an embodiment, the polynucleotide comprises (a) a 5′ UTR comprising the sequence of SEQ ID NO: 8 or a variant or fragment thereof; (b) a coding region comprising a stop element comprising the sequence of SEQ ID NO: 26; (c) a 3′ UTR comprising the sequence of SEQ ID NO: 19; and (d) a poly-A tail, e.g., a poly-A tail comprising the sequence of SEQ ID NO: 50.

In an embodiment, the polynucleotide comprises (a) a 5′ UTR comprising the sequence of SEQ ID NO: 1 or a variant or fragment thereof; (b) a coding region comprising a stop element comprising the sequence of SEQ ID NO: 26; (c) a 3′ UTR comprising the sequence of SEQ ID NO: 19; and (d) a poly-A tail, e.g., a poly-A tail comprising the sequence of SEQ ID NO: 50.

In an embodiment, the polynucleotide comprises (a) a 5′ UTR comprising the sequence of SEQ ID NO: 1 or a variant or fragment thereof, (b) a coding region comprising a stop element comprising the sequence of SEQ ID NO: 26; (c) a 3′ UTR comprising the sequence of SEQ ID NO: 19; and (d) a poly-A tail, e.g., a poly-A tail comprising the sequence of SEQ ID NO: 50.

In an embodiment, the polynucleotide comprises (a) a 5′ UTR comprising the sequence of SEQ ID NO: 8 or a variant or fragment thereof; (b) a coding region comprising a stop element comprising the sequence of SEQ ID NO: 33; (c) a 3′ UTR comprising the sequence of SEQ ID NO: 19; and (d) a poly-A tail, e.g., a poly-A tail comprising the sequence of SEQ ID NO: 50.

In an embodiment, the polynucleotide comprises (a) a 5′ UTR comprising the sequence of SEQ ID NO: 1 or a variant or fragment thereof; (b) a coding region comprising a stop element comprising the sequence of SEQ ID NO: 33; (c) a 3′ UTR comprising the sequence of SEQ ID NO: 19; and (d) a poly-A tail, e.g., a poly-A tail comprising the sequence of SEQ ID NO: 50.

In an embodiment, the polynucleotide comprises (a) a 5′ UTR comprising the sequence of SEQ ID NO: 1 or a variant or fragment thereof; (b) a coding region comprising a stop element comprising the sequence of SEQ ID NO: 33; (c) a 3′ UTR comprising the sequence of SEQ ID NO: 19; and (d) a poly-A tail, e.g., a poly-A tail comprising the sequence of SEQ ID NO: 50.

In an embodiment, the polynucleotide comprises (a) a 5′ UTR comprising the sequence of SEQ ID NO: 1 or a variant or fragment thereof; (b) a coding region comprising a stop element comprising the sequence of SEQ ID NO: 26; (c) a 3′ UTR comprising a TENT recruiting element (e.g., the sequence of SEQ ID NO: 91 or 92), e.g., a 3′ UTR comprising the sequence of SEQ ID NO: 80; and (d) a poly-A tail, e.g., a poly-A tail comprising one or more guanosine residues, optionally wherein the poly-A tail is 100 nucleotides in length.

In an embodiment, the polynucleotide comprises (a) a 5′ UTR comprising the sequence of SEQ ID NO: 1 or a variant or fragment thereof; (b) a coding region comprising a stop element comprising the sequence of SEQ ID NO: 26; (c) a 3′ UTR comprising three copies of a TENT recruiting element (e.g., the sequence of SEQ ID NO: 91 or 92); and (d) a poly-A tail, e.g., a poly-A tail comprising one or more guanosine residues, optionally wherein the poly-A tail is 100 nucleotides in length.

In an embodiment, the polynucleotide comprises (a) a 5′ UTR comprising the sequence of SEQ ID NO: 1 or a variant or fragment thereof; (b) a coding region comprising a stop element comprising the sequence of SEQ ID NO: 33; (c) a 3′ UTR comprising a TENT recruiting element (e.g., the sequence of SEQ ID NO: 91 or 92), e.g., a 3′ UTR comprising the sequence of SEQ ID NO: 80; and (d) a poly-A tail, e.g., a poly-A tail comprising one or more guanosine residues, optionally wherein the poly-A tail is 100 nucleotides in length.

In an embodiment, the polynucleotide comprises (a) a 5′ UTR comprising the sequence of SEQ ID NO: 66 or a variant or fragment thereof, (b) a coding region comprising a stop element comprising the sequence of SEQ ID NO: 33; (c) a 3′ UTR comprising a TENT recruiting element (e.g., the sequence of SEQ ID NO: 91 or 92), e.g., a 3′ UTR comprising the sequence of SEQ ID NO: 94; and (d) a poly-A tail, e.g., a poly-A tail comprising one or more guanosine residues, optionally wherein the poly-A tail is 100 nucleotides in length.

In an embodiment, the polynucleotide comprises (a) a 5′ UTR comprising the sequence of SEQ ID NO: 66 or a variant or fragment thereof, (b) a coding region comprising a stop element comprising the sequence of SEQ ID NO: 33; (c) a 3′ UTR comprising a TENT recruiting element (e.g., the sequence of SEQ ID NO: 91 or 92), e.g., a 3′ UTR comprising the sequence of SEQ ID NO: 94; and (d) a poly-A tail, e.g., a poly-A tail comprising one or more guanosine residues, optionally wherein the poly-A tail is 100 nucleotides in length.

In an embodiment, the polynucleotide comprises (a) a 5′ UTR comprising the sequence of SEQ ID NO: 1 or a variant or fragment thereof; (b) a coding region comprising a stop element comprising the sequence of SEQ ID NO: 26; (c) a 3′ UTR comprising the sequence of SEQ ID NO: 19; and (d) a poly-A tail, e.g., a poly-A tail comprising a 3′ stabilizing region comprising an inverted thymidine (idT).

In an embodiment, the polynucleotide comprises (a) a 5′ UTR comprising the sequence of SEQ ID NO: 1 or a variant or fragment thereof; (b) a coding region comprising a stop element comprising the sequence of SEQ ID NO: 26; (c) a 3′ UTR comprising a TENT recruiting element (e.g., the sequence of SEQ ID NO: 91 or 92), e.g., a 3′ UTR comprising the sequence of SEQ ID NO: 80; and (d) a poly-A tail, e.g., a poly-A tail comprising one or more guanosine residues and a 3′ stabilizing region comprising an inverted thymidine (idT), optionally wherein the poly-A tail is 100 nucleotides in length.

In an embodiment, the polynucleotide comprises (a) a 5′ UTR comprising the sequence of SEQ ID NO: 66 or a variant or fragment thereof, (b) a coding region comprising a stop element comprising the sequence of SEQ ID NO: 33; (c) a 3′ UTR comprising a TENT recruiting element (e.g., the sequence of SEQ ID NO: 91 or 92), e.g., a 3′ UTR comprising the sequence of SEQ ID NO: 94; and (d) a poly-A tail, e.g., a poly-A tail comprising one or more guanosine residues and a 3′ stabilizing region comprising an inverted thymidine (idT), optionally wherein the poly-A tail is 100 nucleotides in length.

In an embodiment, the polynucleotide comprises (a) a 5′ UTR comprising the sequence of SEQ ID NO: 1 or a variant or fragment thereof, and (b) a 3′ UTR comprising the sequence of SEQ ID NO: 11 or a variant or fragment thereof.

In an embodiment, the polynucleotide comprises (a) a 5′ UTR comprising the sequence of SEQ ID NO: 1 or a variant or fragment thereof, and (b) a coding region comprising a stop element comprising the sequence of SEQ ID NO: 26.

In an embodiment, the polynucleotide comprises (a) a 5′ UTR comprising the sequence of SEQ ID NO: 1 or a variant or fragment thereof, and (b) a coding region comprising a stop element comprising the sequence of SEQ ID NO: 29.

In an embodiment, the polynucleotide comprises (a) a 5′ UTR comprising the sequence of SEQ ID NO: 1 or a variant or fragment thereof, and (b) a coding region comprising a stop element comprising the sequence of SEQ ID NO: 30.

In an embodiment, the polynucleotide comprises (a) a 5′ UTR comprising the sequence of SEQ ID NO: 1 or a variant or fragment thereof, and (b) a coding region comprising a stop element comprising the sequence of SEQ ID NO: 32.

In an embodiment, the polynucleotide comprises (a) a 5′ UTR comprising the sequence of SEQ ID NO: 1 or a variant or fragment thereof, and (b) a coding region comprising a stop element comprising the sequence of SEQ ID NO: 33.

In an embodiment, the polynucleotide comprises (a) a 5′ UTR comprising the sequence of SEQ ID NO: 8 or a variant or fragment thereof, and (b) a coding region comprising a stop element comprising the sequence of SEQ ID NO: 26.

In an embodiment, the polynucleotide comprises (a) a 5′ UTR comprising the sequence of SEQ ID NO: 8 or a variant or fragment thereof, and (b) a coding region comprising a stop element comprising the sequence of SEQ ID NO: 29.

In an embodiment, the polynucleotide comprises (a) a 5′ UTR comprising the sequence of SEQ ID NO: 8 or a variant or fragment thereof, and (b) a coding region comprising a stop element comprising the sequence of SEQ ID NO: 30.

In an embodiment, the polynucleotide comprises (a) a 5′ UTR comprising the sequence of SEQ ID NO: 8 or a variant or fragment thereof, and (b) a coding region comprising a stop element comprising the sequence of SEQ ID NO: 32.

In an embodiment, the polynucleotide comprises (a) a 5′ UTR comprising the sequence of SEQ ID NO: 8 or a variant or fragment thereof, and (b) a coding region comprising a stop element comprising the sequence of SEQ ID NO: 33.

In an embodiment, the polynucleotide comprises (a) a 5′ UTR comprising the sequence of SEQ ID NO: 42 or a variant or fragment thereof, and (b) a coding region comprising a stop element comprising the sequence of SEQ ID NO: 26.

In an embodiment, the polynucleotide comprises (a) a 5′ UTR comprising the sequence of SEQ ID NO: 42 or a variant or fragment thereof, and (b) a coding region comprising a stop element comprising the sequence of SEQ ID NO: 29.

In an embodiment, the polynucleotide comprises (a) a 5′ UTR comprising the sequence of SEQ ID NO: 42 or a variant or fragment thereof, and (b) a coding region comprising a stop element comprising the sequence of SEQ ID NO: 30.

In an embodiment, the polynucleotide comprises (a) a 5′ UTR comprising the sequence of SEQ ID NO: 42 or a variant or fragment thereof, and (b) a coding region comprising a stop element comprising the sequence of SEQ ID NO: 32.

In an embodiment, the polynucleotide comprises (a) a 5′ UTR comprising the sequence of SEQ ID NO: 42 or a variant or fragment thereof, and (b) a coding region comprising a stop element comprising the sequence of SEQ ID NO: 33.

In an embodiment, a polynucleotide of the disclosure comprises (a) a 5′ UTR described in Table 1 or a variant or fragment thereof and (c) a 3′ UTR described in Table 2 or a variant or fragment thereof. In an embodiment, the polynucleotide further comprises a cap structure, e.g., as described herein, or a poly A tail, e.g., as described herein. In an embodiment, the polynucleotide further comprises a 3′ stabilizing region, e.g., as described herein.

In an embodiment, a polynucleotide of the disclosure comprises (c) a 3′ UTR described in Table 2 or a variant or fragment thereof and (b) a coding region comprising a stop element provided in Table 3. In an embodiment, the polynucleotide comprises a sequence provided in Table 4. In an embodiment, the polynucleotide comprises a 3′ UTR with a stop element as described in Table 4. In an embodiment, the polynucleotide comprise a sequence with at least 80%, 85%, 90%, 95%, 96%, 97%, 98%, 99% or 100% identity to any of SEQ ID NOs: 47, 48, 49, 50, 122, 52, 53, 54, 55, 59, 60, 61, 126, 127, 97, 98, 99, 100, 101, 102, 103, 104, 105, 106, 107, 108, 109, 110, 111, 112, 113, 114, 115, 116, 117, 118, 119, or 120, or a variant or fragment thereof. In an embodiment, the polynucleotide further comprises a cap structure, e.g., as described herein, or a poly A tail, e.g., as described herein. In an embodiment, the polynucleotide further comprises a 3′ stabilizing region, e.g., as described herein.

In an embodiment, a polynucleotide of the disclosure comprises (a) a 5′ UTR described in Table 1 or a variant or fragment thereof, (b) a coding region comprising a stop element provided in Table 3; and (c) a 3′ UTR described in Table 2 or a variant or fragment thereof. In an embodiment, the polynucleotide further comprises a cap structure, e.g., as described herein, or a poly A tail, e.g., as described herein. In an embodiment, the polynucleotide further comprises a 3′ stabilizing region, e.g., as described herein.

TABLE 4
Exemplary 3′ UTR and stop element sequences

| SEQ ID | Sequence | |
|---|---|---|
| NO | information | Sequence |
| 47 | 3′ UTR with stop | UAGGGUUAAGCUGGAGCCUCGGUGGCCUAGCUUCUUGCCC |
| | C11 (underlined) | CUUGGGCCUCCCCCCAGCCCCUCCUCCCCUUCCUGCACCC |
| | | GUACCCCCGUGGUCUUUGAAUAAAGUCUGAGUGGGCGGC |
| 48 | 3′ UTR with stop | UAAAGCUCCGCUGGAGCCUCGGUGGCCUAGCUUCUUGCCC |
| | C10 (underlined) | CUUGGGCCUCCCCCCAGCCCCUCCUCCCCUUCCUGCACCC |
| | | GUACCCCCGUGGUCUUUGAAUAAAGUCUGAGUGGGCGGC |
| 49 | 3′ UTR with stop | UAAGCCCCUGCUGGAGCCUCGGUGGCCUAGCUUCUUGCCC |
| | C9 (underlined) | CUUGGGCCUCCCCCCAGCCCCUCCUCCCCUUCCUGCACCC |
| | | GUACCCCCGUGGUCUUUGAAUAAAGUCUGAGUGGGCGGC |
| 50 | 3′ UTR with stop | UAAGCACCCGCUGGAGCCUCGGUGGCCUAGCUUCUUGCCC |
| | C8 (underlined) | CUUGGGCCUCCCCCCAGCCCCUCCUCCCCUUCCUGCACCC |
| | | GUACCCCCGUGGUCUUUGAAUAAAGUCUGAGUGGGCGGC |
| 122 | 3′ UTR with stop | UAAGCCCCUCCGGGGGCCUCGGUGGCCUAGCUUCUUGCCC |
| | C7 (underlined) | CUUGGGCCUCCCCCCAGCCCCUCCUCCCCUUCCUGCACCC |
| | | GUACCCCCGUGGUCUUUGAAUAAAGUCUGAGUGGGCGGC |
| 52 | 3′ UTR with stop | UAAGGCUAAGCUGGAGCCUCGGUGGCCUAGCUUCUUGCCC |
| | C6 (underlined) | CUUGGGCCUCCCCCCAGCCCCUCCUCCCCUUCCUGCACCC |
| | | GUACCCCCGUGGUCUUUGAAUAAAGUCUGAGUGGGCGGC |
| 53 | 3′ UTR with stop | UAAGUCUCCGCUGGAGCCUCGGUGGCCUAGCUUCUUGCCC |
| | C5 (underlined) | CUUGGGCCUCCCCCCAGCCCCUCCUCCCCUUCCUGCACCC |
| | | GUACCCCCGUGGUCUUUGAAUAAAGUCUGAGUGGGCGGC |
| 54 | 3′ UTR with stop | UAAAGCUAAGCUGGAGCCUCGGUGGCCUAGCUUCUUGCCC |
| | C4 (underlined) | CUUGGGCCUCCCCCCAGCCCCUCCUCCCCUUCCUGCACCC |
| | | GUACCCCCGUGGUCUUUGAAUAAAGUCUGAGUGGGCGGC |
| 55 | 3′ UTR with stop | UAAGUCUAAGCUGGAGCCUCGGUGGCCUAGCUUCUUGCCC |
| | C3 (underlined) | CUUGGGCCUCCCCCCAGCCCCUCCUCCCCUUCCUGCACCC |
| | | GUACCCCCGUGGUCUUUGAAUAAAGUCUGAGUGGGCGGC |
| 59 | 3′ UTR with C10 | UAAAGCUCCAUAAAGUAGGAAACACUACAGCUGGAGCCUC |
| | stop (underlined) | GGUGGCCUAGCUUCUUGCCCCUUGGGCCUCCAUAAAGUAG |
| | | GAAACACUACAUCCCCCCAGCCCCUCCUCCCCUUCCUGCA |
| | | CCCGUACCCCCUCCAUAAAGUAGGAAACACUACAGUGGUC |
| | | UUUGAAUAAAGUCUGAGUGGGCGGC |
| 60 | 3′ UTR with C7 | UAAGCCCCUCCGGGGUCCAUAAAGUAGGAAACACUACAGC |
| | stop (underlined) | CUCGGUGGCCUAGCUUCUUGCCCCUUGGGCCUCCAUAAAG |
| | | UAGGAAACACUACAUCCCCCCAGCCCCUCCUCCCCUUCCU |
| | | GCACCCGUACCCCCUCCAUAAAGUAGGAAACACUACAGUG |
| | | GUCUUUGAAUAAAGUCUGAGUGGGCGGC |
| 61 | 3′ UTR with C8 | UAAAGCUCCCCGGGGUCCAUAAAGUAGGAAACACUACAGC |
| | stop (underlined) | CUCGGUGGCCUAGCUUCUUGCCCCUUGGGCCUCCAUAAAG |
| | | UAGGAAACACUACAUCCCCCCAGCCCCUCCUCCCCUUCCU |
| | | GCACCCGUACCCCCUCCAUAAAGUAGGAAACACUACAGUG |
| | | GUCUUUGAAUAAAGUCUGAGUGGGCGGC |
| 126 | 3′ UTR with C8 | UAAAGCUCCCCGGGGGCCUCCACCGCGUUAUCCGUUCCUC |
| | stop (underlined) | GUAGGCUGGUCCUGGGGAACGGGUCGGCGGGUACCCCCGU |
| | | GGUCUUUGAAUAAAGUCUGAGUGGGCGGC |
| 127 | 3′ UTR with C8 | UAAAGCUCCCCGGGGCACCGCGUUAUCCGUUCCUCGUAGG |
| | stop (underlined) | CUGGUCCUGGGGAACGGGUCGGCGGGCCUCGGUGGCCUAG |
| | | CUUCUUGCCCCUUGGGCCCACCGCGUUAUCCGUUCCUCGU |
| | | AGGCUGGUCCUGGGGAACGGGUCGGCGGUCCCCCCAGCCC |
| | | CUCCUCCCCUUCCUGCACCCGUACCCCCCACCGCGUUAUC |
| | | CGUUCCUCGUAGGCUGGUCCUGGGGAACGGGUCGGCGGGU |
| | | GGUCUUUGAAUAAAGUCUGAGUGGGCGGC |
| 97 | 3′ UTR with C2 | UAAUAGUAAGCUGGAGCCUCCUGAGAGACCUGUGUGAACU |
| | stop (underlined) | AUUGAGAAGAUCGGAACAGCUCCUUACUCUGAGGAAGUUG |
| | | GUACCCCCGUGGUCUUUGAAUAAAGUCUGAGUGGGCGGC |
| 98 | 3′ UTR with C2 | UAAUAGUAAGCUGGAGCCUCACUCUCCUCUCCAUCCCGUA |
| | stop (underlined) | UCCAGGCUGUGAAUUUUUCAAGGAAUAUAAAGAUCGGGAU |
| | | GUACCCCCGUGGUCUUUGAAUAAAGUCUGAGUGGGCGGC |
| 99 | 3′ UTR with C16 | UGAUAGUAAGCUGGAGCCUCUAGUGACGGCAACAGGGCUU |
| | stop (underlined) | GGUUUUUCCUUGUUGUGAAAUCGACAUCUCUGAAGACAGG |
| | | GUACCCCCGUGGUCUUUGAAUAAAGUCUGAGUGGGCGGC |
| 100 | 3′ UTR with C16 | UGAUAGUAAGCUGGAGCCUCCUUCCAUCUAGUCACAAAGA |
| | stop (underlined) | CUCCUUCGUCCCCAGUUGCCGUCUAGGAUUGGGCCUCCCA |
| | | GUACCCCCGUGGUCUUUGAAUAAAGUCUGAGUGGGCGGC |
| 101 | 3′ UTR with C16 | UGAUAGUAAGCUGGAGCCUCCCAUAACAUGACAUAUCUGG |
| | stop (underlined) | AUUUUGUGCUUAGAACCUUAAAUUGGAAGCAUUCUUAAUU |
| | | GUACCCCCGUGGUCUUUGAAUAAAGUCUGAGUGGGCGGC |
| 102 | 3′ UTR with C2 | UAAUAGUAAGCUGGAGCCUCCGGAAAACUAAAAUAGAGAU |
| | stop (underlined) | AUUUCAAGAUUUUAUAAUUUUCAAAGACCUUUGAAAUAUU |
| | | GUACCCCCGUGGUCUUUGAAUAAAGUCUGAGUGGGCGGC |
| 103 | 3′ UTR with C2 | UAAUAGUAAGCUGGAGCCUCUACACAUUGCUUCUAGUUGG |
| | stop (underlined) | CAGAAAUAAUUGAUUAAAAGACCAGAAACUGUGAUAACUG |
| | | GUACCCCCGUGGUCUUUAAAUAAAGUCUAAGUGGGCGGC |
| 104 | 3′ UTR with C1 | UGAUAAUAGGCUGGAGCCUCGGUGGCCAUGCUUCUUGCCC |
| | stop (underlined) | CUUGGGCCUCCCCCCAGCCCCUCCUCCCCUUCCUGCACCC |
| | | GUACCCCCGUGGUCUUUGAAUAAAGUCUGAGUGGGCGGC |
| 105 | 3′ UTR with C1 | UGAUAAUAGGCUGGAGCCUCGGUGGCCUAGCUUCUUGCCC |
| | stop (underlined) | CUUGGGCCUCCCCCCAGCCCCUCCUCCCCUUCCUGCACCC |
| | | GUACCCCCGUGGUCUUUGAAUAAAGUCUGAGUGGGCGGC |
| 106 | 3′ UTR with C1 | UGAUAAUAGGCUGGAGCCUCGGUGGCCAUGCUUCUUGCCC |
| | stop (underlined) | CUUGGGCCUCCCCCCAGCCCCUCCUCCCCUUCCUGCACCC |
| | | GUACCCCCCAAACACCAUUGUCACACUCCAGUGGUCUUUG |
| | | AAUAAAGUCUGAGUGGGCGGC |
| 107 | 3′ UTR with C1 | UGAUAAUAGGCUGGAGCCUCGGUGGCCUAGCUUCUUGCCC |
| | stop (underlined) | CUUGGGCCUCCCCCCAGCCCCUCCUCCCCUUCCUGCACCC |
| | | GUACCCCCCAAACACCAUUGUCACACUCCAGUGGUCUUUG |
| | | AAUAAAGUCUGAGUGGGCGGC |
| 108 | 3′ UTR with C1 | UGAUAAUAGGCUGGAGCCUCGGUGGCCUAGCUUCUUGCCC |
| | stop (underlined) | CUUGGGCCUCCCCCCAGCCCCUCCUCCCCUUCCUGCACCC |
| | | GUACCCCCUCCAUAAAGUAGGAAACACUACAGUGGUCUUU |
| | | GAAUAAAGUCUGAGUGGGCGGC |
| 109 | 3′ UTR with C1 | UGAUAAUAGGCUGGAGCCUCGGUGGCCUAGCUUCUUGCCC |
| | stop (underlined) | CUUGGGCCUCCCCCCAGCCCCUCCUCCCCUUCCUGCACCC |
| | | GUACCCCCCGCAUUAUUACUCACGGUACGAGUGGUCUUUG |
| | | AAUAAAGUCUGAGUGGGCGGC |
| 110 | 3′ UTR with C1 | UGAUAAUAGUCCAUAAAGUAGGAAACACUACAGCUGGAGC |
| | stop (underlined) | CUCGGUGGCCAUGCUUCUUGCCCCUUGGGCCUCCCCCCAG |
| | | CCCCUCCUCCCCUUCCUGCACCCGUACCCCCCGCAUUAUU |
| | | ACUCACGGUACGAGUGGUCUUUGAAUAAAGUCUGAGUGGG |
| | | CGGC |
| 111 | 3′ UTR with C1 | UGAUAAUAGUCCAUAAAGUAGGAAACACUACAGCUGGAGC |
| | stop (underlined) | CUCGGUGGCCUAGCUUCUUGCCCCUUGGGCCUCCAUAAAG |
| | | UAGGAAACACUACAUCCCCCCAGCCCCUCCUCCCCUUCCU |
| | | GCACCCGUACCCCCUCCAUAAAGUAGGAAACACUACAGUG |
| | | GUCUUUGAAUAAAGUCUGAGUGGGCGGC |
| 112 | 3′ UTR with C1 | UGAUAAUAGGCUGGAGCCUCUCACACACCUCUGCCCCUUG |
| | stop (underlined) | GGCCUCCCACUCCCAUGGCUCUGGGCGGUCCAGAAGGAGC |
| | | GUACCCCCGUGGUCUUUGAAUAAAGUCUGAGUGGGCGGC |
| 113 | 3′ UTR with C1 | UGAUAAUAGGCUGGAGCCUCCACCGCGUUAUCCGUUCCUC |
| | stop (underlined) | GUAGGCUGGUCCUGGGGAACGGGUCGGCGGGUACCCCCGU |
| | | GGUCUUUGAAUAAAGUCUGAGUGGGCGGC |
| 114 | 3′ UTR with C1 | UGAUAAUAGGCUGGAGCCUCUGCCCGGCAACGGCCAGGUC |
| | stop (underlined) | UGUGCCAAGUGUUUGCUGACGCAACCCCCACUGGCUGGGG |
| | | CUUGGUCAUGGGCCAUCAGCGCGUGCGUGGAACCUUUUCG |
| | | GCUCCUCUGCCGAUCCAUACUGCGGAACUCCUAGCCGCUU |
| | | GUUUUGCUCGCAGCAGGUCUGGAGCAAACAUUAUCGGGAC |
| | | UGAUAACUCUGUUGUCCUGUACCCCCGUGGUCUUUGAAUA |
| | | AAGUCUGAGUGGGCGGC |
| 115 | 3′ UTR with C1 | UGAUAAUAGGCUGGAGCCUCGGUGGCCUAGCUUCUUGCCC |
| | stop (underlined) | CUUGGGCCUCCCCCCAGCCCCUCCUCCCCUUCCUGCACCC |
| | | GUACCCUUUUUUUUUUUUUUUUUUUCUUCUUUUCUUUUUU |
| | | UUCUUUUUUUUUUUUCUUUCUUUUUUUCUUUUUUUUUCUU |
| | | UUCUUUUUUCUUUUUUUUUUUUUUUUCCGUGGUCUUUGAA |
| | | UAAAGUCUGAGUGGGCGGC |
| 116 | 3′ UTR with C1 | UGAUAAUAGGCUGGAGCCUCGGUGGCCUAGCUUCUUGCCC |
| | stop (underlined) | CUUGGGCCUCCCCCCAGCCCCUCCUCCCCUUCCUGCACCC |
| | | GUACCcuuuuuuuuuuuuuuuuuuuuuuuuuuuuuuuuuu |
| | | UUUUUUUUUUUUUUUUUUUUUUUUUUUUUUUUUUUUUUUU |
| | | UUUUUUUUUUUUUUUUUUUUUUUUUUCCGUGGUCUUUGAA |
| | | UAAAGUCUGAGUGGGCGGC |
| 117 | 3′ UTR with stop | UAAGUCUCCAUAAAGUAGGAAACACUACAGCUGGAGCCUC |
| | C5 (underlined) | GGUGGCCUAGCUUCUUGCCCCUUGGGCCUCCAUAAAGUAG |
| | | GAAACACUACAUCCCCCCAGCCCCUCCUCCCCUUCCUGCA |
| | | CCCGUACCCCCUCCAUAAAGUAGGAAACACUACAGUGGUC |
| | | UUUGAAUAAAGUCUGAGUGGGCGGC |
| 118 | 3′ UTR with C17 | UAAAGCGCUGGAGCCUCGGUGGCCUAGCUUCUUGCCCCUU |
| | stop (underlined) | GGGCCUCCCCCCAGCCCCUCCUCCCCUUCCUGCACCCGUA |
| | | CCCCCUCCAUAAAGUAGGAAACACUACAGUGGUCUUUGAA |
| | | UAAAGUCUGAGUGGGCGGC |
| 119 | 3′ UTR with C8 | UAAAGCUCCCCGGGGUCCAUAAAGUAGGAAACACUACAGC |
| | stop (underlined) | UGGAGCCUCCUGAGAGACCUGUGUGAACUAUUGAGAAGAU |
| | | CGGAACAGCUCCUUACUCUGAGGAAGUUGUCCAUAAAGUA |
| | | GGAAACACUACAGUACCCCCUCCAUAAAGUAGGAAACACU |
| | | ACAGUGGUCUUUGAAUAAAGUCUGAGUGGGCGGC |
| 120 | 3′ UTR with C2 | UAAUAGUAAACCUCACUCACGGCCACAUUGAGUGCCAGGC |
| | stop (underlined) | UCCGGGCUGGUUUAUAGUAGUGUAGAGCAUUGCAGCACUU |
| | | AGACUGGGGUGCUGUAGUCUUUAUUGUAGUCUUUCCACAU |
| | | ACCUGAUAAUUCUUAGAUAAUUUCUUAUUUUAAUUCCAUA |
| | | AAGUAGGAAACACUACAUAAAUCUCCAUAAAGUAGGAAAC |
| | | ACUACAUAUUCUUCCAUAAAGUAGGAAACACUACAUAGGC |
| | | U |

In an embodiment, the polynucleotide comprises (a) a coding region comprising a stop element comprising the sequence of SEQ ID NO: 36; and (b) a 3′ UTR comprising the sequence of SEQ ID NO: 19 or a variant or fragment thereof. In an embodiment, the polynucleotide comprises the sequence of SEQ ID NO: 47 or a variant or fragment thereof.

In an embodiment, the polynucleotide comprises (a) a coding region comprising a stop element comprising the sequence of SEQ ID NO: 35; and (b) a 3′ UTR comprising the sequence of SEQ ID NO: 19 or a variant or fragment thereof. In an embodiment, the polynucleotide comprises the sequence of SEQ ID NO: 48 or a variant or fragment thereof.

In an embodiment, the polynucleotide comprises (a) a coding region comprising a stop element comprising the sequence of SEQ ID NO: 34; and (b) a 3′ UTR comprising the sequence of SEQ ID NO: 19 or a variant or fragment thereof. In an embodiment, the polynucleotide comprises the sequence of SEQ ID NO: 49 or a variant or fragment thereof.

In an embodiment, the polynucleotide comprises (a) a coding region comprising a stop element comprising the sequence of SEQ ID NO: 33; and (b) a 3′ UTR comprising the sequence of SEQ ID NO: 19 or a variant or fragment thereof. In an embodiment, the polynucleotide comprises the sequence of SEQ ID NO: 50 or a variant or fragment thereof.

In an embodiment, the polynucleotide comprises (a) a coding region comprising a stop element comprising the sequence of SEQ ID NO: 32; and (b) a 3′ UTR comprising the sequence of SEQ ID NO: 19 or a variant or fragment thereof. In an embodiment, the polynucleotide comprises the sequence of SEQ ID NO: 122 or a variant or fragment thereof.

In an embodiment, the polynucleotide comprises (a) a coding region comprising a stop element comprising the sequence of SEQ ID NO: 31; and (b) a 3′ UTR comprising the sequence of SEQ ID NO: 19 or a variant or fragment thereof. In an embodiment, the polynucleotide comprises the sequence of SEQ ID NO: 52 or a variant or fragment thereof.

In an embodiment, the polynucleotide comprises (a) a coding region comprising a stop element comprising the sequence of SEQ ID NO: 30; and (b) a 3′ UTR comprising the sequence of SEQ ID NO: 19 or a variant or fragment thereof. In an embodiment, the polynucleotide comprises the sequence of SEQ ID NO: 53 or a variant or fragment thereof.

In an embodiment, the polynucleotide comprises (a) a coding region comprising a stop element comprising the sequence of SEQ ID NO: 29; and (b) a 3′ UTR comprising the sequence of SEQ ID NO: 19 or a variant or fragment thereof. In an embodiment, the polynucleotide comprises the sequence of SEQ ID NO: 54 or a variant or fragment thereof.

In an embodiment, the polynucleotide comprises (a) a coding region comprising a stop element comprising the sequence of SEQ ID NO: 28; and (b) a 3′ UTR comprising the sequence of SEQ ID NO: 19 or a variant or fragment thereof. In an embodiment, the polynucleotide comprises the sequence of SEQ ID NO: 55 or a variant or fragment thereof.

In an embodiment, the polynucleotide comprises (a) a coding region comprising a stop element comprising the sequence of SEQ ID NO: 35; and (b) a 3′ UTR comprising the sequence of SEQ ID NO: 19 or a variant or fragment thereof. In an embodiment, the polynucleotide comprises the sequence of SEQ ID NO: 59 or a variant or fragment thereof.

In an embodiment, the polynucleotide comprises (a) a coding region comprising a stop element comprising the sequence of SEQ ID NO: 32; and (b) a 3′ UTR comprising nucleotides 16-188 of SEQ ID NO: 60 or a variant or fragment thereof. In an embodiment, the polynucleotide comprises the sequence of SEQ ID NO: 60 or a variant or fragment thereof.

In an embodiment, the polynucleotide comprises (a) a coding region comprising a stop element comprising the sequence of SEQ ID NO: 33; and (b) a 3′ UTR comprising nucleotides 16-188 of SEQ ID NO: 61 or a variant or fragment thereof. In an embodiment, the polynucleotide comprises the sequence of SEQ ID NO: 61.

In an embodiment, the polynucleotide comprises (a) a coding region comprising a stop element comprising the sequence of SEQ ID NO: 33; and (b) a 3′ UTR comprising the sequence of SEQ ID NO: 94 or a variant or fragment thereof. In an embodiment, the polynucleotide comprises the sequence of SEQ ID NO: 126.

In an embodiment, the polynucleotide comprises (a) a coding region comprising a stop element comprising the sequence of SEQ ID NO: 33; and (b) a 3′ UTR comprising the sequence of SEQ ID NO: 95 or a variant or fragment thereof. In an embodiment, the polynucleotide comprises the sequence of SEQ ID NO: 127.

In an embodiment, the polynucleotide comprises (a) a coding region comprising a stop element comprising the sequence of SEQ ID NO: 27; and (b) a 3′ UTR comprising the sequence of SEQ ID NO: 11 or a variant or fragment thereof. In an embodiment, the polynucleotide comprises the sequence of SEQ ID NO: 97.

In an embodiment, the polynucleotide comprises (a) a coding region comprising a stop element comprising the sequence of SEQ ID NO: 27; and (b) a 3′ UTR comprising the sequence of SEQ ID NO: 12 or a variant or fragment thereof. In an embodiment, the polynucleotide comprises the sequence of SEQ ID NO: 98.

In an embodiment, the polynucleotide comprises (a) a coding region comprising a stop element comprising the sequence of SEQ ID NO: 25; and (b) a 3′ UTR comprising the sequence of SEQ ID NO: 13 or a variant or fragment thereof. In an embodiment, the polynucleotide comprises the sequence of SEQ ID NO: 99.

In an embodiment, the polynucleotide comprises (a) a coding region comprising a stop element comprising the sequence of SEQ ID NO: 25; and (b) a 3′ UTR comprising the sequence of SEQ ID NO: 14 or a variant or fragment thereof. In an embodiment, the polynucleotide comprises the sequence of SEQ ID NO: 100.

In an embodiment, the polynucleotide comprises (a) a coding region comprising a stop element comprising the sequence of SEQ ID NO: 25; and (b) a 3′ UTR comprising the sequence of SEQ ID NO: 15 or a variant or fragment thereof. In an embodiment, the polynucleotide comprises the sequence of SEQ ID NO: 101.

In an embodiment, the polynucleotide comprises (a) a coding region comprising a stop element comprising the sequence of SEQ ID NO: 27; and (b) a 3′ UTR comprising the sequence of SEQ ID NO: 16 or a variant or fragment thereof. In an embodiment, the polynucleotide comprises the sequence of SEQ ID NO: 102.

In an embodiment, the polynucleotide comprises (a) a coding region comprising a stop element comprising the sequence of SEQ ID NO: 27; and (b) a 3′ UTR comprising the sequence of SEQ ID NO: 17 or a variant or fragment thereof. In an embodiment, the polynucleotide comprises the sequence of SEQ ID NO: 103.

In an embodiment, the polynucleotide comprises (a) a coding region comprising a stop element comprising the sequence of SEQ ID NO: 26; and (b) a 3′ UTR comprising the sequence of SEQ ID NO: 18 or a variant or fragment thereof. In an embodiment, the polynucleotide comprises the sequence of SEQ ID NO: 104.

In an embodiment, the polynucleotide comprises (a) a coding region comprising a stop element comprising the sequence of SEQ ID NO: 26; and (b) a 3′ UTR comprising the sequence of SEQ ID NO: 19 or a variant or fragment thereof. In an embodiment, the polynucleotide comprises the sequence of SEQ ID NO: 105.

In an embodiment, the polynucleotide comprises (a) a coding region comprising a stop element comprising the sequence of SEQ ID NO: 26; and (b) a 3′ UTR comprising the sequence of SEQ ID NO: 20 or a variant or fragment thereof. In an embodiment, the polynucleotide comprises the sequence of SEQ ID NO: 106.

In an embodiment, the polynucleotide comprises (a) a coding region comprising a stop element comprising the sequence of SEQ ID NO: 26; and (b) a 3′ UTR comprising the sequence of SEQ ID NO: 21 or a variant or fragment thereof. In an embodiment, the polynucleotide comprises the sequence of SEQ ID NO: 107.

In an embodiment, the polynucleotide comprises (a) a coding region comprising a stop element comprising the sequence of SEQ ID NO: 26; and (b) a 3′ UTR comprising the sequence of SEQ ID NO: 22 or a variant or fragment thereof. In an embodiment, the polynucleotide comprises the sequence of SEQ ID NO: 108.

In an embodiment, the polynucleotide comprises (a) a coding region comprising a stop element comprising the sequence of SEQ ID NO: 26; and (b) a 3′ UTR comprising the sequence of SEQ ID NO: 23 or a variant or fragment thereof. In an embodiment, the polynucleotide comprises the sequence of SEQ ID NO: 109.

In an embodiment, the polynucleotide comprises (a) a coding region comprising a stop element comprising the sequence of SEQ ID NO: 26; and (b) a 3′ UTR comprising the sequence of SEQ ID NO: 24 or a variant or fragment thereof. In an embodiment, the polynucleotide comprises the sequence of SEQ ID NO: 110.

In an embodiment, the polynucleotide comprises (a) a coding region comprising a stop element comprising the sequence of SEQ ID NO: 26; and (b) a 3′ UTR comprising the sequence of SEQ ID NO: 25 or a variant or fragment thereof. In an embodiment, the polynucleotide comprises the sequence of SEQ ID NO: 111.

In an embodiment, the polynucleotide comprises (a) a coding region comprising a stop element comprising the sequence of SEQ ID NO: 26; and (b) a 3′ UTR comprising the sequence of SEQ ID NO: 79 or a variant or fragment thereof. In an embodiment, the polynucleotide comprises the sequence of SEQ ID NO: 112.

In an embodiment, the polynucleotide comprises (a) a coding region comprising a stop element comprising the sequence of SEQ ID NO: 26; and (b) a 3′ UTR comprising the sequence of SEQ ID NO: 80 or a variant or fragment thereof. In an embodiment, the polynucleotide comprises the sequence of SEQ ID NO: 113.

In an embodiment, the polynucleotide comprises (a) a coding region comprising a stop element comprising the sequence of SEQ ID NO: 26; and (b) a 3′ UTR comprising the sequence of SEQ ID NO: 81 or a variant or fragment thereof. In an embodiment, the polynucleotide comprises the sequence of SEQ ID NO: 114.

In an embodiment, the polynucleotide comprises (a) a coding region comprising a stop element comprising the sequence of SEQ ID NO: 26; and (b) a 3′ UTR comprising the sequence of SEQ ID NO: 82 or a variant or fragment thereof. In an embodiment, the polynucleotide comprises the sequence of SEQ ID NO: 115.

In an embodiment, the polynucleotide comprises (a) a coding region comprising a stop element comprising the sequence of SEQ ID NO: 26; and (b) a 3′ UTR comprising the sequence of SEQ ID NO: 83 or a variant or fragment thereof. In an embodiment, the polynucleotide comprises the sequence of SEQ ID NO: 116.

In an embodiment, the polynucleotide comprises (a) a coding region comprising a stop element comprising the sequence of SEQ ID NO: 30; and (b) a 3′ UTR comprising the sequence of SEQ ID NO: 84 or a variant or fragment thereof. In an embodiment, the polynucleotide comprises the sequence of SEQ ID NO: 117.

In an embodiment, the polynucleotide comprises (a) a coding region comprising a stop element comprising the sequence of SEQ ID NO: 96; and (b) a 3′ UTR comprising the sequence of SEQ ID NO: 22 or a variant or fragment thereof. In an embodiment, the polynucleotide comprises the sequence of SEQ ID NO: 118.

In an embodiment, the polynucleotide comprises (a) a coding region comprising a stop element comprising the sequence of SEQ ID NO: 33; and (b) a 3′ UTR comprising the sequence of SEQ ID NO: 86 or a variant or fragment thereof. In an embodiment, the polynucleotide comprises the sequence of SEQ ID NO: 119.

In an embodiment, the polynucleotide comprises (a) a coding region comprising a stop element comprising the sequence of SEQ ID NO: 27; and (b) a 3′ UTR comprising the sequence of SEQ ID NO: 87 or a variant or fragment thereof. In an embodiment, the polynucleotide comprises the sequence of SEQ ID NO: 120.

### Therapeutic Payload or Prophylactic Payload

Disclosed herein, inter alia, is a polynucleotide having a 5′ UTR described herein, a 3′ UTR described herein, and/or a coding region comprising a stop element, which coding region further comprises a sequence that encodes for a payload, e.g., a therapeutic payload or a prophylactic payload. In an embodiment, the coding region encodes for one payload. In an embodiment, the coding region encodes for more than one payload, e.g., 2, 3, 4, 5, 6, or more payloads, e.g., same or different payloads. In an embodiment, the sequence encoding each payload is contiguous in the polynucleotide. In an embodiment, the sequence encoding each payload is separated by at least 1-1000 nucleotides. In some embodiments, the therapeutic payload or prophylactic payload comprises an mRNA encoding: a secreted protein; a membrane-bound protein; or an intercellular protein, or peptides, polypeptides or biologically active fragments thereof.

Also disclosed herein is an LNP comprising a polynucleotide comprising a coding region which encodes for a payload, e.g., a therapeutic payload or a prophylactic payload. In some embodiments, the therapeutic payload or prophylactic payload comprises an mRNA encoding: a secreted protein; a membrane-bound protein; or an intercellular protein, or peptides, polypeptides or biologically active fragments thereof.

In some embodiments, the therapeutic payload or prophylactic payload comprises an mRNA encoding a secreted protein, or a peptide, a polypeptide or a biologically active fragment thereof. In some embodiments, the secreted protein comprises a cytokine, or a variant or fragment (e.g., a biologically active fragment) thereof. In some embodiments, the secreted protein comprises an antibody or a variant or fragment (e.g., a biologically active fragment) thereof. In some embodiments, the secreted protein comprises an enzyme or a variant or fragment (e.g., a biologically active fragment) thereof. In some embodiments, the secreted protein comprises a hormone or a variant or fragment (e.g., a biologically active fragment) thereof. In some embodiments, the secreted protein comprises a ligand, or a variant or fragment (e.g., a biologically active fragment) thereof. In some embodiments, the secreted protein comprises a vaccine (e.g., an antigen, an immunogenic epitope), or a component, variant or fragment (e.g., a biologically active fragment) thereof. In some embodiments, the vaccine is a prophylactic vaccine. In some embodiments, the vaccine is a therapeutic vaccine, e.g., a cancer vaccine. In some embodiments, the secreted protein comprises a growth factor or a component, variant or fragment (e.g., a biologically active fragment) thereof. In some embodiments, the secreted protein comprises an immune modulator, e.g., an immune checkpoint agonist or antagonist.

In some embodiments, the therapeutic payload or prophylactic payload comprises an mRNA encoding a membrane-bound protein, or a peptide, a polypeptide or a biologically active fragment thereof. In some embodiments, the membrane-bound protein comprises a vaccine (e.g., an antigen, an immunogenic epitope), or a component, variant or fragment (e.g., a biologically active fragment) thereof. In some embodiments, the vaccine is a prophylactic vaccine. In some embodiments, the vaccine is a therapeutic vaccine, e.g., a cancer vaccine. In some embodiments, the membrane-bound protein comprises a ligand, a variant or fragment (e.g., a biologically active fragment) thereof. In some embodiments, the membrane-bound protein comprises a membrane transporter, a variant or fragment (e.g., a biologically active fragment) thereof. In some embodiments, the membrane-bound protein comprises a structural protein, a variant or fragment (e.g., a biologically active fragment) thereof. In some embodiments, the membrane-bound protein comprises an immune modulator, e.g., an immune checkpoint agonist or antagonist.

In some embodiments, the therapeutic payload or prophylactic payload comprises an mRNA encoding an intracellular protein, or a peptide, a polypeptide or a biologically active fragment thereof. In some embodiments, the intracellular protein comprises an enzyme, or a variant or fragment (e.g., a biologically active fragment) thereof. In some embodiments, the intracellular protein comprises a transcription factor, or a variant or fragment (e.g., a biologically active fragment) thereof. In some embodiments, the intracellular protein comprises a nuclease, or a variant or fragment (e.g., a biologically active fragment) thereof. In some embodiments, the intracellular protein comprises a structural protein, or a variant or fragment (e.g., a biologically active fragment) thereof.

In some embodiments, the therapeutic payload or prophylactic payload is chosen from a cytokine, an antibody, a vaccine (e.g., an antigen, an immunogenic epitope), a receptor, an enzyme, a hormone, a transcription factor, a ligand, a membrane transporter, a structural protein, a nuclease, a growth factor, an immune modulator, or a component, variant or fragment (e.g., a biologically active fragment) thereof.

In some embodiments, the therapeutic payload or prophylactic payload comprises a protein or peptide.

It will be understood that the regulatory elements disclosed herein (e.g., 5′ UTRs, stop elements, 3′ UTRs, stabilizing regions (e.g., idT or modified poly A tails) can be used with ORFs encoding a payload described herein. It will further be understood that the regulatory elements disclosed herein can be used in a modular fashion, i.e., can be used in an mRNA construct in combination with other regulatory elements from the art (e.g., a 5′ UTR of the instant invention in combination with an ORF and other regulatory regions from the art), or can be used in combination with the other regulatory elements disclosed herein (e.g., a 5′ UTR of the instant invention and a 3′ UTR of the instant invention, et cetera). It will further be understood that a stop element of the present invention can be used in combination with a desired ORF that lacks a stop codon. It will also be understood that when a desired ORF comprises a stop codon, an additional stop codon or stop element will not be included in the final construct. In some embodiments, the stop codon in the desired ORF can be replaced with a stop element described herein.

Micro RNA (miRNA) Binding Sites

Nucleic acid molecules (e.g., RNA, e.g., mRNA) of the disclosure can include regulatory elements, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof.

In some embodiments, a nucleic acid molecule (e.g., RNA, e.g., mRNA) of the disclosure comprises an open reading frame (ORF) encoding a polypeptide of interest and further comprises one or more miRNA binding site(s). Inclusion or incorporation of miRNA binding site(s) provides for regulation of nucleic acid molecules (e.g., RNA, e.g., mRNA) of the disclosure, and in turn, of the polypeptides encoded therefrom, based on tissue-specific and/or cell-type specific expression of naturally-occurring miRNAs.

A miRNA, e.g., a natural-occurring miRNA, is a 19-25 nucleotide long noncoding RNA that binds to a nucleic acid molecule (e.g., RNA, e.g., mRNA) and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA binding site is flanked by an adenosine (A) opposed to miRNA position 1. In some embodiments, a miRNA seed can comprise 6 nucleotides (e.g., nucleotides 2-7 of the mature miRNA), wherein the seed-complementary site in the corresponding miRNA binding site is flanked by an adenosine (A) opposed to miRNA position 1. See, for example, Grimson A, Farh K K, Johnston W K, Garrett-Engele P, Lim L P, Bartel D P; Mol Cell. 2007 Jul. 6; 27(1):91-105. miRNA profiling of the target cells or tissues can be conducted to determine the presence or absence of miRNA in the cells or tissues. In some embodiments, a nucleic acid molecule (e.g., RNA, e.g., mRNA) of the disclosure comprises one or more microRNA binding sites, microRNA target sequences, microRNA complementary sequences, or microRNA seed complementary sequences. Such sequences can correspond to, e.g., have complementarity to, any known microRNA such as those taught in US Publication US2005/0261218 and US Publication US2005/0059005, the contents of each of which are incorporated herein by reference in their entirety.

As used herein, the term “microRNA (miRNA or miR) binding site” refers to a sequence within a nucleic acid molecule, e.g., within a DNA or within an RNA transcript, including in the 5′ UTR and/or 3′ UTR, that has sufficient complementarity to all or a region of a miRNA to interact with, associate with or bind to the miRNA. In some embodiments, a nucleic acid molecule (e.g., RNA, e.g., mRNA) of the disclosure comprising an ORF encoding a polypeptide of interest and further comprises one or more miRNA binding site(s). In exemplary embodiments, a 5′ UTR and/or 3′ UTR of the nucleic acid molecule (e.g., RNA, e.g., mRNA) comprises the one or more miRNA binding site(s).

A miRNA binding site having sufficient complementarity to a miRNA refers to a degree of complementarity sufficient to facilitate miRNA-mediated regulation of a nucleic acid molecule (e.g., RNA, e.g., mRNA), e.g., miRNA-mediated translational repression or degradation of the nucleic acid molecule (e.g., RNA, e.g., mRNA). In exemplary aspects of the disclosure, a miRNA binding site having sufficient complementarity to the miRNA refers to a degree of complementarity sufficient to facilitate miRNA-mediated degradation of the nucleic acid molecule (e.g., RNA, e.g., mRNA), e.g., miRNA-guided RNA-induced silencing complex (RISC)-mediated cleavage of mRNA. The miRNA binding site can have complementarity to, for example, a 19-25 nucleotide miRNA sequence, to a 19-23 nucleotide miRNA sequence, or to a 22 nucleotide miRNA sequence. A miRNA binding site can be complementary to only a portion of a miRNA, e.g., to a portion less than 1, 2, 3, or 4 nucleotides of the full length of a naturally-occurring miRNA sequence. Full or complete complementarity (e.g., full complementarity or complete complementarity over all or a significant portion of the length of a naturally-occurring miRNA) is preferred when the desired regulation is mRNA degradation.

In some embodiments, a miRNA binding site includes a sequence that has complementarity (e.g., partial or complete complementarity) with a miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with an miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs are still capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the nucleic acid molecule (e.g., RNA, e.g., mRNA) comprising the miRNA binding site. In other embodiments, the miRNA binding site has imperfect complementarity so that a RISC complex comprising the miRNA induces instability in the nucleic acid molecule (e.g., RNA, e.g., mRNA) comprising the miRNA binding site. In another embodiment, the miRNA binding site has imperfect complementarity so that a RISC complex comprising the miRNA represses transcription of the nucleic acid molecule (e.g., RNA, e.g., mRNA) comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a nucleic acid molecule (e.g., RNA, e.g., mRNA) of the disclosure, the nucleic acid molecule (e.g., RNA, e.g., mRNA) can be targeted for degradation or reduced translation, provided the miRNA in question is available. This can reduce off-target effects upon delivery of the nucleic acid molecule (e.g., RNA, e.g., mRNA). For example, if a nucleic acid molecule (e.g., RNA, e.g., mRNA) of the disclosure is not intended to be delivered to a tissue or cell but ends up is said tissue or cell, then a miRNA abundant in the tissue or cell can inhibit the expression of the gene of interest if one or multiple binding sites of the miRNA are engineered into the 5′ UTR and/or 3′ UTR of the nucleic acid molecule (e.g., RNA, e.g., mRNA).

For example, one of skill in the art would understand that one or more miR binding sites can be included in a nucleic acid molecule (e.g., RNA, e.g., mRNA) to minimize expression in cell types other than lymphoid cells. In one embodiment, a miR122 binding site can be used. In another embodiment, a miR126 binding site can be used. In still another embodiment, multiple copies of these miR binding sites or combinations may be used.

Conversely, miRNA binding sites can be removed from nucleic acid molecule (e.g., RNA, e.g., mRNA) sequences in which they naturally occur in order to increase protein expression in specific tissues. For example, a binding site for a specific miRNA can be removed from a nucleic acid molecule (e.g., RNA, e.g., mRNA) to improve protein expression in tissues or cells containing the miRNA.

Regulation of expression in multiple tissues can be accomplished through introduction or removal of one or more miRNA binding sites, e.g., one or more distinct miRNA binding sites. The decision whether to remove or insert a miRNA binding site can be made based on miRNA expression patterns and/or their profilings in tissues and/or cells in development and/or disease. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

miRNAs and miRNA binding sites can correspond to any known sequence, including non-limiting examples described in U.S. Publication Nos. 2014/0200261, 2005/0261218, and 2005/0059005, each of which are incorporated herein by reference in their entirety.

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126).

Specifically, miRNAs are known to be differentially expressed in immune cells (also called hematopoietic cells), such as antigen presenting cells (APCs) (e.g., dendritic cells and monocytes), monocytes, monocytes, B lymphocytes, T lymphocytes, granulocytes, natural killer cells, etc. Immune cell specific miRNAs are involved in immunogenicity, autoimmunity, the immune response to infection, inflammation, as well as unwanted immune response after gene therapy and tissue/organ transplantation. Immune cell specific miRNAs also regulate many aspects of development, proliferation, differentiation and apoptosis of hematopoietic cells (immune cells). For example, miR-142 and miR-146 are exclusively expressed in immune cells, particularly abundant in myeloid dendritic cells. It has been demonstrated that the immune response to a nucleic acid molecule (e.g., RNA, e.g., mRNA) can be shut-off by adding miR-142 binding sites to the 3′-UTR of the polynucleotide, enabling more stable gene transfer in tissues and cells. miR-142 efficiently degrades exogenous nucleic acid molecules (e.g., RNA, e.g., mRNA) in antigen presenting cells and suppresses cytotoxic elimination of transduced cells (e.g., Annoni A et al., blood, 2009, 114, 5152-5161; Brown B D, et al., Nat med. 2006, 12(5), 585-591; Brown B D, et al., blood, 2007, 110(13): 4144-4152, each of which is incorporated herein by reference in its entirety).

An antigen-mediated immune response can refer to an immune response triggered by foreign antigens, which, when entering an organism, are processed by the antigen presenting cells and displayed on the surface of the antigen presenting cells. T cells can recognize the presented antigen and induce a cytotoxic elimination of cells that express the antigen.

Introducing a miR-142 binding site into the 5′ UTR and/or 3′ UTR of a nucleic acid molecule of the disclosure can selectively repress gene expression in antigen presenting cells through miR-142 mediated degradation, limiting antigen presentation in antigen presenting cells (e.g., dendritic cells) and thereby preventing antigen-mediated immune response after the delivery of the nucleic acid molecule (e.g., RNA, e.g., mRNA). The nucleic acid molecule (e.g., RNA, e.g., mRNA) is then stably expressed in target tissues or cells without triggering cytotoxic elimination.

In one embodiment, binding sites for miRNAs that are known to be expressed in immune cells, in particular, antigen presenting cells, can be engineered into a nucleic acid molecule (e.g., RNA, e.g., mRNA) of the disclosure to suppress the expression of the nucleic acid molecule (e.g., RNA, e.g., mRNA) in antigen presenting cells through miRNA mediated RNA degradation, subduing the antigen-mediated immune response. Expression of the nucleic acid molecule (e.g., RNA, e.g., mRNA) is maintained in non-immune cells where the immune cell specific miRNAs are not expressed. For example, in some embodiments, to prevent an immunogenic reaction against a liver specific protein, any miR-122 binding site can be removed and a miR-142 (and/or mirR-146) binding site can be engineered into the 5′ UTR and/or 3′ UTR of a nucleic acid molecule of the disclosure.

To further drive the selective degradation and suppression in APCs and macrophage, a nucleic acid molecule (e.g., RNA, e.g., mRNA) of the disclosure can include a further negative regulatory element in the 5′ UTR and/or 3′ UTR, either alone or in combination with miR-142 and/or miR-146 binding sites. As a non-limiting example, the further negative regulatory element is a Constitutive Decay Element (CDE).

Immune cell specific miRNAs include, but are not limited to, hsa-let-7a-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1-3p, hsa-let-7f-2-5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p, miR-24-2-5p, miR-24-3p, miR-26a-1-3p, miR-26a-2-3p, miR-26a-5p, miR-26b-3p, miR-26b-5p, miR-27a-3p, miR-27a-5p, miR-27b-3p, miR-27b-5p, miR-28-3p, miR-28-5p, miR-2909, miR-29a-3p, miR-29a-5p, miR-29b-1-5p, miR-29b-2-5p, miR-29c-3p, miR-29c-5p, miR-30e-3p, miR-30e-5p, miR-331-5p, miR-339-3p, miR-339-5p, miR-345-3p, miR-345-5p, miR-346, miR-34a-3p, miR-34a-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p, and miR-99b-5p. Furthermore, novel miRNAs can be identified in immune cell through micro-array hybridization and microtome analysis (e.g., Jima D D et al, Blood, 2010, 116:e118-e127; Vaz C et al., BMC Genomics, 2010, 11, 288, the content of each of which is incorporated herein by reference in its entirety.) In some embodiments, a miRNA binding site is inserted in the nucleic acid molecule (e.g., RNA, e.g., mRNA) of the disclosure in any position of the nucleic acid molecule (e.g., RNA, e.g., mRNA) (e.g., the 5′ UTR and/or 3′ UTR). In some embodiments, the 5′ UTR comprises a miRNA binding site. In some embodiments, the 3′ UTR comprises a miRNA binding site. In some embodiments, the 5′ UTR and the 3′ UTR comprise a miRNA binding site. The insertion site in the nucleic acid molecule (e.g., RNA, e.g., mRNA) can be anywhere in the nucleic acid molecule (e.g., RNA, e.g., mRNA) as long as the insertion of the miRNA binding site in the nucleic acid molecule (e.g., RNA, e.g., mRNA) does not interfere with the translation of a functional polypeptide in the absence of the corresponding miRNA; and in the presence of the miRNA, the insertion of the miRNA binding site in the nucleic acid molecule (e.g., RNA, e.g., mRNA) and the binding of the miRNA binding site to the corresponding miRNA are capable of degrading the polynucleotide or preventing the translation of the nucleic acid molecule (e.g., RNA, e.g., mRNA).

In some embodiments, a miRNA binding site is inserted in at least about 30 nucleotides downstream from the stop codon of an ORF in a nucleic acid molecule (e.g., RNA, e.g., mRNA) of the disclosure comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in a polynucleotide of the disclosure. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in a nucleic acid molecule (e.g., RNA, e.g., mRNA) of the disclosure. miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′ UTR and/or 3′ UTR. As a non-limiting example, a non-human 3′ UTR can increase the regulatory effect of the miRNA sequence on the expression of a polypeptide of interest compared to a human 3′ UTR of the same sequence type.

In one embodiment, other regulatory elements and/or structural elements of the 5′ UTR can influence miRNA mediated gene regulation. One example of a regulatory element and/or structural element is a structured IRES (Internal Ribosome Entry Site) in the 5′ UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The nucleic acid molecules (e.g., RNA, e.g., mRNA) of the disclosure can further include this structured 5′ UTR in order to enhance microRNA mediated gene regulation.

At least one miRNA binding site can be engineered into the 3′ UTR of a polynucleotide of the disclosure. In this context, at least two, at least three, at least four, at least five, at least six, at least seven, at least eight, at least nine, at least ten, or more miRNA binding sites can be engineered into a 3′ UTR of a nucleic acid molecule (e.g., RNA, e.g., mRNA) of the disclosure. For example, 1 to 10, 1 to 9, 1 to 8, 1 to 7, 1 to 6, 1 to 5, 1 to 4, 1 to 3, 2, or 1 miRNA binding sites can be engineered into the 3′ UTR of a nucleic acid molecule (e.g., RNA, e.g., mRNA) of the disclosure. In one embodiment, miRNA binding sites incorporated into a nucleic acid molecule (e.g., RNA, e.g., mRNA) of the disclosure can be the same or can be different miRNA sites. A combination of different miRNA binding sites incorporated into a nucleic acid molecule (e.g., RNA, e.g., mRNA) of the disclosure can include combinations in which more than one copy of any of the different miRNA sites are incorporated. In another embodiment, miRNA binding sites incorporated into a nucleic acid molecule (e.g., RNA, e.g., mRNA) of the disclosure can target the same or different tissues in the body. As a non-limiting example, through the introduction of tissue-, cell-type-, or disease-specific miRNA binding sites in the 3′-UTR of a nucleic acid molecule (e.g., RNA, e.g., mRNA) of the disclosure, the degree of expression in specific cell types (e.g., hepatocytes, myeloid cells, endothelial cells, cancer cells, etc.) can be reduced.

In one embodiment, a miRNA binding site can be engineered near the 5′ terminus of the 3′ UTR, about halfway between the 5′ terminus and 3′ terminus of the 3′ UTR and/or near the 3′ terminus of the 3′ UTR in a nucleic acid molecule (e.g., RNA, e.g., mRNA) of the disclosure. As a non-limiting example, a miRNA binding site can be engineered near the 5′ terminus of the 3′ UTR and about halfway between the 5′ terminus and 3′ terminus of the 3′ UTR. As another non-limiting example, a miRNA binding site can be engineered near the 3′ terminus of the 3′ UTR and about halfway between the 5′ terminus and 3′ terminus of the 3′ UTR. As yet another non-limiting example, a miRNA binding site can be engineered near the 5′ terminus of the 3′ UTR and near the 3′ terminus of the 3′ UTR.

In another embodiment, a 3′ UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

A nucleic acid molecule (e.g., RNA, e.g., mRNA) of the disclosure can be engineered for more targeted expression in specific tissues, cell types, or biological conditions based on the expression patterns of miRNAs in the different tissues, cell types, or biological conditions. Through introduction of tissue-specific miRNA binding sites, a nucleic acid molecule (e.g., RNA, e.g., mRNA) of the disclosure can be designed for optimal protein expression in a tissue or cell, or in the context of a biological condition.

In some embodiments, a nucleic acid molecule (e.g., RNA, e.g., mRNA) of the disclosure can comprise at least one miRNA binding site in the 3′ UTR in order to selectively degrade mRNA therapeutics in the immune cells to subdue unwanted immunogenic reactions caused by therapeutic delivery. As a non-limiting example, the miRNA binding site can make a nucleic acid molecule (e.g., RNA, e.g., mRNA) of the disclosure more unstable in antigen presenting cells. Non-limiting examples of these miRNAs include mir-142-5p, mir-142-3p, mir-146a-5p, and mir-146-3p.

Additional Features of 5′ UTRs and 3′ UTRs A UTR can be homologous or heterologous to the coding region in a polynucleotide. In some embodiments, the UTR is homologous to the ORF encoding the therapeutic payload or prophylactic payload. In some embodiments, the UTR is heterologous to the ORF encoding the therapeutic payload or prophylactic payload.

In some embodiments, the polynucleotide comprises two or more 5′ UTRs or functional fragments thereof, each of which has the same or different nucleotide sequences. In some embodiments, the polynucleotide comprises two or more 3′ UTRs or functional fragments thereof, each of which has the same or different nucleotide sequences.

In some embodiments, the 5′ UTR or functional fragment thereof, 3′ UTR or functional fragment thereof, or any combination thereof is sequence optimized.

In some embodiments, the 5′ UTR or functional fragment thereof, 3′ UTR or functional fragment thereof, or any combination thereof comprises at least one chemically modified nucleobase, e.g., N1-methylpseudouracil or 5-methoxyuracil.

UTRs can have features that provide a regulatory role, e.g., increased or decreased stability, localization and/or translation efficiency. A polynucleotide comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively. Natural 5′ UTRs bear features that play roles in translation initiation. They harbor signatures like Kozak sequences that are commonly known to be involved in the process by which the ribosome initiates translation of many genes. Kozak sequences have the consensus CCR(A/G)CCAUGG (SEQ ID NO: 125), where R is a purine (adenine or guanine) three bases upstream of the start codon (AUG), which is followed by another ‘G’. 5′ UTRs also have been known to form secondary structures that are involved in elongation factor binding.

By engineering the features typically found in abundantly expressed genes of specific target organs, one can enhance the stability and protein production of a polynucleotide. For example, introduction of 5′ UTR of liver-expressed mRNA, such as albumin, serum amyloid A, Apolipoprotein A/B/E, transferrin, alpha fetoprotein, erythropoietin, or Factor VIII, can enhance expression of polynucleotides in hepatic cell lines or liver. Likewise, use of 5′ UTR from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin) and for lung epithelial cells (e.g., SP-A/B/C/D).

In some embodiments, UTRs are selected from a family of transcripts whose proteins share a common function, structure, feature or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new polynucleotide. In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

Co-owned International Patent Application No. PCT/US2014/021522 (Publ. No. WO/2014/164253, incorporated herein by reference in its entirety) provides a listing of exemplary UTRs that can be utilized in the polynucleotide of the present invention as flanking regions to an ORF.

Additional exemplary UTRs of the application include, but are not limited to, one or more 5′ UTR and/or 3′ UTR derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human a or R actin); a GAPDH; a tubulin; a histone; a citric acid cycle enzyme; a topoisomerase (e.g., a 5′ UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the R subunit of mitochondrial H⁺-ATP synthase); a growth hormone e (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

In some embodiments, the 5′ UTR is selected from the group consisting of a β-globin 5′ UTR; a 5′ UTR containing a strong Kozak translational initiation signal; a cytochrome b-245 α polypeptide (CYBA) 5′ UTR; a hydroxysteroid (17-β) dehydrogenase (HSD17B4) 5′ UTR; a Tobacco etch virus (TEV) 5′ UTR; a Venezuelen equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′ UTR; a DEN 3′ UTR; a PAV barley yellow dwarf virus (BYDV-PAV) 3′ UTR; an elongation factor 1 α1 (EEF1A1) 3′ UTR; a manganese superoxide dismutase (MnSOD) 3′ UTR; a β subunit of mitochondrial H(+)-ATP synthase (β-mRNA) 3′ UTR; a GLUT1 3′ UTR; a MEF2A 3′ UTR; a β-F1-ATPase 3′ UTR; functional fragments thereof and combinations thereof.

Wild-type UTRs derived from any gene or mRNA can be incorporated into the polynucleotides of the invention. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, the contents of which are incorporated herein by reference in their entirety.

UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs. In some embodiments, the polynucleotide comprises multiple UTRs, e.g., a double, a triple or a quadruple 5′ UTR or 3′ UTR. For example, a double UTR comprises two copies of the same UTR either in series or substantially in series. For example, a double beta-globin 3′ UTR can be used (see US2010/0129877, the contents of which are incorporated herein by reference in its entirety).

The polynucleotides of the invention can comprise combinations of features. For example, the ORF can be flanked by a 5′ UTR that comprises a strong Kozak translational initiation signal and/or a 3′ UTR comprising an oligo(dT) sequence for templated addition of a poly-A tail. A 5′ UTR can comprise a first polynucleotide fragment and a second polynucleotide fragment from the same and/or different UTRs (see, e.g., US2010/0293625, herein incorporated by reference in its entirety).

Other non-UTR sequences can be used as regions or subregions within the polynucleotides of the invention. For example, introns or portions of intron sequences can be incorporated into the polynucleotides of the invention. Incorporation of intronic sequences can increase protein production as well as polynucleotide expression levels. In some embodiments, the polynucleotide of the invention comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the polynucleotide comprises an IRES instead of a 5′ UTR sequence. In some embodiments, the polynucleotide comprises an ORF and a viral capsid sequence. In some embodiments, the polynucleotide comprises a synthetic 5′ UTR in combination with a non-synthetic 3′ UTR.

In some embodiments, the UTR can also include at least one translation enhancer polynucleotide, translation enhancer element, or translational enhancer elements (collectively, “TEE,” which refers to nucleic acid sequences that increase the amount of polypeptide or protein produced from a polynucleotide. As a non-limiting example, the TEE can be located between the transcription promoter and the start codon. In some embodiments, the 5′ UTR comprises a TEE. In one aspect, a TEE is a conserved element in a UTR that can promote translational activity of a nucleic acid such as, but not limited to, cap-dependent or cap-independent translation.

### Nucleotide Caps

The disclosure also includes a polynucleotide that comprises both a 5′ Cap and a polynucleotide of the present invention (e.g., a polynucleotide comprising a nucleotide sequence encoding a polypeptide to be expressed).

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or anteterminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

In some embodiments, the polynucleotides of the present invention (e.g., a polynucleotide comprising a nucleotide sequence encoding a polypeptide) incorporate a cap moiety.

In some embodiments, polynucleotides of the present invention comprise a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, Mass.) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide that functions as an mRNA molecule. Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs can be chemically (i.e., non-enzymatically) or enzymatically synthesized and/or linked to the polynucleotides of the invention.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m⁷G-3′mppp-G; which can equivalently be designated 3′ O-Me-m⁷G(5′)ppp(5′)G). The 3′-O atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

Another exemplary cap is m⁷G-ppp-Gm-A (i.e., N7,guanosine-5′-triphosphate-2′-O-dimethyl-guanosine-adenosine).

In some embodiments, the cap is a dinucleotide cap analog. As a non-limiting example, the dinucleotide cap analog can be modified at different phosphate positions with a boranophosphate group or a phosphoroselenoate group such as the dinucleotide cap analogs described in U.S. Pat. No. 8,519,110, the contents of which are herein incorporated by reference in its entirety.

In another embodiment, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m^{3′-O}G(5′)ppp(5′)G cap analog (See, e.g., the various cap analogs and the methods of synthesizing cap analogs described in Kore et al. Bioorganic & Medicinal Chemistry 2013 21:4570-4574; the contents of which are herein incorporated by reference in its entirety). In another embodiment, a cap analog of the present invention is a 4-chloro/bromophenoxyethyl analog.

Polynucleotides of the invention can also be capped post-manufacture (whether IVT or chemical synthesis), using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′ cap structures of the present invention are those that, among other things, have enhanced binding of cap binding proteins, increased half-life, reduced susceptibility to 5′ endonucleases and/or reduced 5′ decapping, as compared to synthetic 5′ cap structures known in the art (or to a wild-type, natural or physiological 5′ cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′ cap analog structures known in the art. Cap structures include, but are not limited to, 7mG(5′)ppp(5′)N1pN2p (cap 0), 7mG(5′)ppp(5′)N1mpNp (cap 1), and 7mG(5′)-ppp(5′)N1mpN2mp (cap 2).

As a non-limiting example, capping chimeric polynucleotides post-manufacture can be more efficient as nearly 100% of the chimeric polynucleotides can be capped. This is in contrast to ˜80% when a cap analog is linked to a chimeric polynucleotide in the course of an in vitro transcription reaction.

According to the present invention, 5′ terminal caps can include endogenous caps or cap analogs. According to the present invention, a 5′ terminal cap can comprise a guanine analog. Useful guanine analogs include, but are not limited to, inosine, N1-methyl-guanosine, 2′ fluoro-guanosine, 7-deaza-guanosine, 8-oxo-guanosine, 2-amino-guanosine, LNA-guanosine, and 2-azido-guanosine.

Also provided herein are exemplary caps including those that can be used in co-transcriptional capping methods for ribonucleic acid (RNA) synthesis, using RNA polymerase, e.g., wild type RNA polymerase or variants thereof, e.g., such as those variants described herein. In one embodiment, caps can be added when RNA is produced in a “one-pot” reaction, without the need for a separate capping reaction. Thus, the methods, in some embodiments, comprise reacting a polynucleotide template with an RNA polymerase variant, nucleoside triphosphates, and a cap analog under in vitro transcription reaction conditions to produce RNA transcript.

As used here the term “cap” includes the inverted G nucleotide and can comprise one or more additional nucleotides 3′ of the inverted G nucleotide, e.g., 1, 2, 3, or more nucleotides 3′ of the inverted G nucleotide and 5′ to the 5′ UTR, e.g., a 5′ UTR described herein.

Exemplary caps comprise a sequence of GG, GA, or GGA, wherein the underlined, italicized G is an in inverted G nucleotide followed by a 5′-5′-triphosphate group.

In one embodiment, a cap comprises a compound of formula (I)

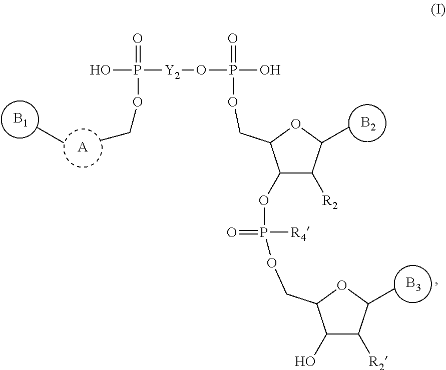

or a stereoisomer, tautomer or salt thereof, wherein

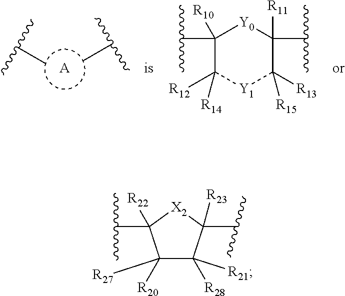

ring B₁ is a modified or unmodified Guanine;

ring B₂ and ring B₃ each independently is a nucleobase or a modified nucleobase;

X₂ is O, S(O)ₚ, NR₂₄ or CR₂₅R₂₆ in which p is 0, 1, or 2;

Y₀ is O or CR₆R₇;

Y₁ is O, S(O)ₙ, CR₆R₇, or NR₈, in which n is 0, 1, or 2;

each - - - is a single bond or absent, wherein when each - - - is a single bond, Yi is O, S(O)ₙ, CR₆R₇, or NR₈; and when each - - - is absent, Y₁ is void;

Y₂ is (OP(O)R₄)ₘ in which m is 0, 1, or 2, or —O—(CR₄₀R₄₁)ᵤ-Q₀-(CR₄₂R₄₃)v-, in which Q₀ is a bond, O, S(O)ᵣ, NR₄₄, or CR₄₅R₄₆, r is 0, 1, or 2, and each of u and v independently is 1, 2, 3 or 4;

each R₂ and R₂′ independently is halo, LNA, or OR₃;

each R₃ independently is H, C₁-C₆ alkyl, C₂-C₆ alkenyl, or C₂-C₆ alkynyl and R₃, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl;

each R₄ and R₄′ independently is H, halo, C₁-C₆ alkyl, OH, SH, SeH, or BH₃⁻;

each of R₆, R₇, and R₈, independently, is -Q₁-T₁, in which Q₁ is a bond or C₁-C₃ alkyl linker optionally substituted with one or more of halo, cyano, OH and C₁-C₆ alkoxy, and T₁ is H, halo, OH, COOH, cyano, or Rₛ₁, in which Rₛ₁ is C₁-C₃ alkyl, C₂-C₆ alkenyl, C₂-C₆ alkynyl, C₁-C₆ alkoxyl, C(O)O—C₁-C₆ alkyl, C₃-C₈ cycloalkyl, C₆-C₁₀ aryl, NR₃₁R₃₂, (NR₃₁R₃₂R₃₃)⁺, 4 to 12-membered heterocycloalkyl, or 5- or 6-membered heteroaryl, and Rₛ₁ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)⁺, C₃-C₈ cycloalkyl, C₆-C₁₀ aryl, 4 to 12-membered heterocycloalkyl, and 5- or 6-membered heteroaryl;

each of R₁₀, R₁₁, R₁₂, R₁₃ R₁₄, and R₁₅, independently, is -Q₂-T₂, in which Q₂ is a bond or C₁-C₃ alkyl linker optionally substituted with one or more of halo, cyano, OH and C₁-C₆ alkoxy, and T₂ is H, halo, OH, NH₂, cyano, NO₂, N₃, Rₛ₂, or ORₛ₂, in which Rₛ₂ is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, NR₃₁R₃₂, (NR₃₁R₃₂R₃₃)⁺, 4 to 12-membered heterocycloalkyl, or 5- or 6-membered heteroaryl, and Rₛ₂ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)⁺, C₃-C₈ cycloalkyl, C₆-C₁₀ aryl, 4 to 12-membered heterocycloalkyl, and 5- or 6-membered heteroaryl; or alternatively R₁₂ together with R₁₄ is oxo, or R₁₃ together with R₁₅ is oxo,

each of R₂₀, R₂₁, R₂₂, and R₂₃ independently is -Q₃-T₃, in which Q₃ is a bond or C₁-C₃ alkyl linker optionally substituted with one or more of halo, cyano, OH and C₁-C₆ alkoxy, and T₃ is H, halo, OH, NH₂, cyano, NO₂, N₃, Rₛ₃, or ORₛ₃, in which Rₛ₃ is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, mono-C₁-C₆ alkylamino, di-C₁-C₆ alkylamino, 4 to 12-membered heterocycloalkyl, or 5- or 6-membered heteroaryl, and Rₛ₃ is optionally substituted with one or more substituents selected from the group consisting of halo, OH, oxo, C₁-C₆ alkyl, COOH, C(O)O—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl;

each of R₂₄, R₂₅, and R₂₆ independently is H or C₁-C₆ alkyl;

each of R₂₇ and R₂₈ independently is H or OR₂₉; or R₂₇ and R₂₈ together form O—R₃₀—O; each R₂₉ independently is H, C₁-C₆ alkyl, C₂-C₆ alkenyl, or C₂-C₆ alkynyl and R₂₉, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl;

R₃₀ is C₁-C₆ alkylene optionally substituted with one or more of halo, OH and C₁-C₆ alkoxyl;

each of R₃₁, R₃₂, and R₃₃, independently is H, C₁-C₆ alkyl, C₃-C₈ cycloalkyl, C₆-C₁₀ aryl, 4 to 12-membered heterocycloalkyl, or 5- or 6-membered heteroaryl;

each of R₄₀, R₄₁, R₄₂, and R₄₃ independently is H, halo, OH, cyano, N₃, OP(O)R₄₇R₄₈, or C₁-C₆ alkyl optionally substituted with one or more OP(O)R₄₇R₄₈, or one R₄₁ and one R₄₃, together with the carbon atoms to which they are attached and Q₀, form C₄-C₁₀ cycloalkyl, 4- to 14-membered heterocycloalkyl, C₆-C₁₀ aryl, or 5- to 14-membered heteroaryl, and each of the cycloalkyl, heterocycloalkyl, phenyl, or 5- to 6-membered heteroaryl is optionally substituted with one or more of OH, halo, cyano, N₃, oxo, OP(O)R₄₇R₄₈, C₁-C₆ alkyl, C₁-C₆ haloalkyl, COOH, C(O)O—C₁-C₆ alkyl, C₁-C₆ alkoxyl, C₁-C₆ haloalkoxyl, amino, mono-C₁-C₆ alkylamino, and di-C₁-C₆ alkylamino;

R₄₄ is H, C₁-C₆ alkyl, or an amine protecting group;

each of R₄₅ and R₄₆ independently is H, OP(O)R₄₇R₄₈, or C₁-C₆ alkyl optionally substituted with one or more OP(O)R₄₇R₄₈, and

each of R₄₇ and R₄₈, independently is H, halo, C₁-C₆ alkyl, OH, SH, SeH, or BH₃⁻.

It should be understood that a cap analog, as provided herein, may include any of the cap analogs described in international publication WO 2017/066797, published on 20 Apr. 2017, incorporated by reference herein in its entirety.

In some embodiments, the B₂ middle position can be a non-ribose molecule, such as arabinose.

In some embodiments R₂ is ethyl-based.

Thus, in some embodiments, a cap comprises the following structure:

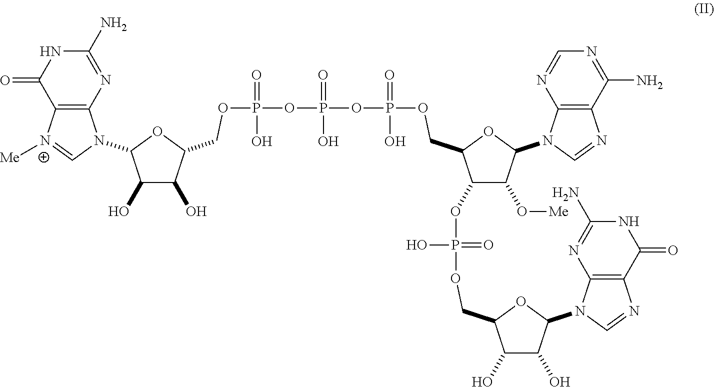

In other embodiments, a cap comprises the following structure:

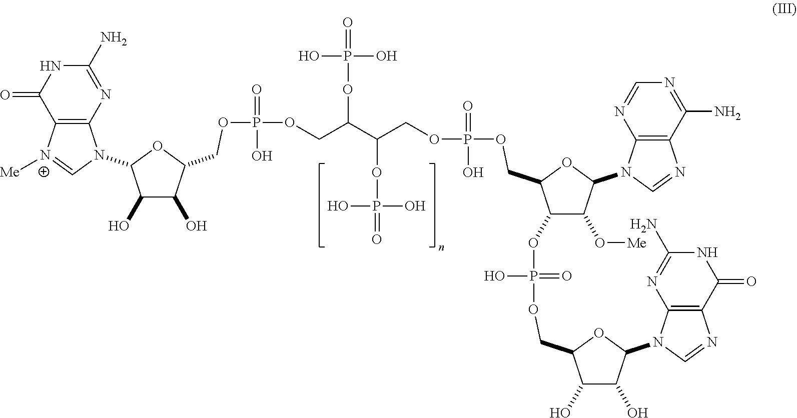

In yet other embodiments, a cap comprises the following structure:

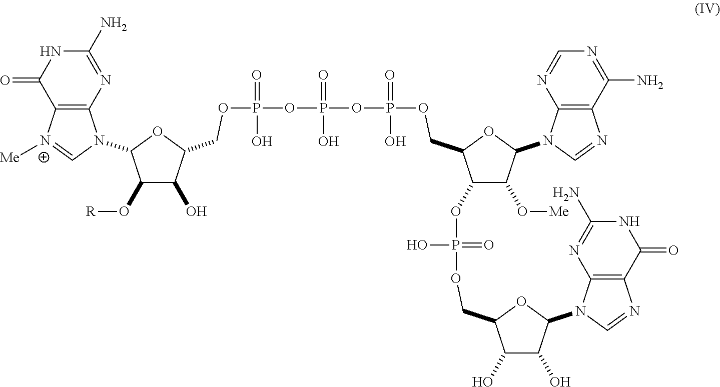

In still other embodiments, a cap comprises the following structure:

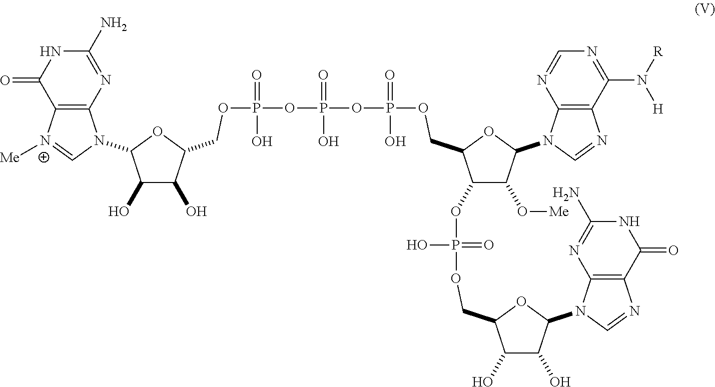

In some embodiments, R is an alkyl (e.g., C₁-C₆ alkyl). In some embodiments, R is a methyl group (e.g., C₁ alkyl). In some embodiments, R is an ethyl group (e.g., C₂ alkyl).

In some embodiments, a cap comprises a sequence selected from the following sequences: GAA, GAC, GAG, GAU, GCA, GCC, GCG, GCU, GGA, GGC, GGG, GGU, GUA, GUC, GUG, and GUU. In some embodiments, a cap comprises GAA. In some embodiments, a cap comprises GAC. In some embodiments, a cap comprises GAG. In some embodiments, a cap comprises GAU. In some embodiments, a cap comprises GCA. In some embodiments, a cap comprises GCC. In some embodiments, a cap comprises GCG. In some embodiments, a cap comprises GCU. In some embodiments, a cap comprises GGA. In some embodiments, a cap comprises GGC. In some embodiments, a cap comprises GGG. In some embodiments, a cap comprises GGU. In some embodiments, a cap comprises GUA. In some embodiments, a cap comprises GUC. In some embodiments, a cap comprises GUG. In some embodiments, a cap comprises GUU.

In some embodiments, a cap comprises a sequence selected from the following sequences: m⁷GpppApA, m⁷GpppApC, m⁷GpppApG, m⁷GpppApU, m⁷GpppCpA, m⁷GpppCpC, m⁷GpppCpG, m⁷GpppCpU, m⁷GpppGpA, m⁷GpppGpC, m⁷GpppGpG, m⁷GpppGpU, m⁷GpppUpA, m⁷GpppUpC, m⁷GpppUpG, and m⁷GpppUpU.

In some embodiments, a cap comprises m⁷GpppApA. In some embodiments, a cap comprises m⁷GpppApC. In some embodiments, a cap comprises m⁷GpppApG. In some embodiments, a cap comprises m⁷GpppApU. In some embodiments, a cap comprises m⁷GpppCpA. In some embodiments, a cap comprises m⁷GpppCpC. In some embodiments, a cap comprises m⁷GpppCpG. In some embodiments, a cap comprises m⁷GpppCpU. In some embodiments, a cap comprises m⁷GpppGpA. In some embodiments, a cap comprises m⁷GpppGpC. In some embodiments, a cap comprises m⁷GpppGpG. In some embodiments, a cap comprises m⁷GpppGpU. In some embodiments, a cap comprises m⁷GpppUpA. In some embodiments, a cap comprises m⁷GpppUpC. In some embodiments, a cap comprises m⁷GpppUpG. In some embodiments, a cap comprises m⁷GpppUpU.

A cap, in some embodiments, comprises a sequence selected from the following sequences: m⁷G_{3′OMe}pppApA, m⁷G_{3′OMe}pppApC, m⁷G_{3′OMe}pppApG, m⁷G_{3′OMe}pppApU, m⁷G_{3′OMe}pppCpA, m⁷G_{3′OMe}pppCpC, m⁷G_{3′OMe}pppCpG, m⁷G_{3′OMe}pppCpU, m⁷G_{3′OMe}pppGpA, m⁷G_{3′OMe}pppGpC, m⁷G_{3′OMe}pppGpG, m⁷G_{3′OMe}pppGpU, m⁷G_{3′OMe}pppUpA, m⁷G_{3′OMe}pppUpC, m⁷G_{3′OMe}pppUpG, and m⁷G_{3′OMe}pppUpU.

In some embodiments, a cap comprises m⁷G_{3′OMe}pppApA. In some embodiments, a cap comprises m⁷G_{3′OMe}pppApC. In some embodiments, a cap comprises m⁷G_{3′OMe}pppApG. In some embodiments, a cap comprises m⁷G_{3′OMe}pppApU. In some embodiments, a cap comprises m⁷G_{3′OMe}pppCpA. In some embodiments, a cap comprises m⁷G_{3′OMe}pppCpC. In some embodiments, a cap comprises m⁷G_{3′OMe}pppCpG. In some embodiments, a cap comprises m⁷G_{3′OMe}pppCpU. In some embodiments, a cap comprises m⁷G_{3′OMe}pppGpA. In some embodiments, a cap comprises m⁷G_{3′OMe}pppGpC. In some embodiments, a cap comprises m⁷G_{3′OMe}pppGpG. In some embodiments, a cap comprises m⁷G_{3′OMe}pppGpU. In some embodiments, a cap comprises m⁷G_{3′OMe}pppUpA. In some embodiments, a cap comprises m⁷G_{3′OMe}pppUpC. In some embodiments, a cap comprises m⁷G_{3′OMe}pppUpG. In some embodiments, a cap comprises m⁷G_{3′OMe}pppUpU.

A cap, in other embodiments, comprises a sequence selected from the following sequences: m⁷G_{3′OMe}pppA_{2′OMe}pA, m⁷G_{3′OMe}pppA_{2′OMe}pC, m⁷G_{3′OMe}pppA_{2′OMe}pG, m⁷G_{3′OMe}pppA_{2′OMe}pU, m⁷G_{3′OMe}pppC_{2′OMe}pA, m⁷G_{3′OMe}pppC_{2′OMe}pC, m⁷G_{3′OMe}pppC_{2′OMe}pG, m⁷G_{3′OMe}pppC_{2′OMe}pU, m⁷G_{3′OMe}pppG_{2′OMe}pA, m⁷G_{3′OMe}pppG_{2′OMe}pC, m⁷G_{3′OMe}pppG_{2′OMe}pG, m⁷G_{3′OMe}pppG_{2′OMe}pU, m⁷G_{3′OMe}pppU_{2′OMe}pA, m⁷G_{3′OMe}pppU_{2′OMe}pC, m⁷G_{3′OMe}pppU_{2′OMe}pG, and m⁷G_{3′OMe}pppU_{2′OMe}pU.

In some embodiments, a cap comprises m⁷G_{3′OMe}pppA_{2′OMe}pA. In some embodiments, a cap comprises m⁷G_{3′OMe}pppA_{2′OMe}pC. In some embodiments, a cap comprises m⁷G_{3′OMe}pppA_{2′OMe}pG. In some embodiments, a cap comprises m⁷G_{3′OMe}pppA_{2′OMe}pU. In some embodiments, a cap comprises m⁷G_{3′OMe}pppC_{2′OMe}pA. In some embodiments, a cap comprises m⁷G_{3′OMe}pppC_{2′OMe}pC. In some embodiments, a cap comprises m⁷G_{3′OMe}pppC_{2′OMe}pG. In some embodiments, a cap comprises m⁷G_{3′OMe}pppC_{2′OMe}pU. In some embodiments, a cap comprises m⁷G_{3′OMe}pppG_{2′OMe}pA. In some embodiments, a cap comprises m⁷G_{3′OMe}pppG_{2′OMe}pC. In some embodiments, a cap comprises m⁷G_{3′OMe}pppG_{2′OMe}pG. In some embodiments, a cap comprises m⁷G_{3′OMe}pppG_{2′OMe}pU. In some embodiments, a cap comprises m⁷G_{3′OMe}pppU_{2′OMe}pA. In some embodiments, a cap comprises m⁷G_{3′OMe}pppU_{2′OMe}pC. In some embodiments, a cap comprises m⁷G_{3′OMe}pppU_{2′OMe}pG. In some embodiments, a cap comprises m⁷G_{3′OMe}pppU_{2′OMe}pU.

A cap, in still other embodiments, comprises a sequence selected from the following sequences: m⁷GpppA_{2′OMe}pA, m⁷GpppA_{2′OMe}pC, m⁷GpppA_{2′OMe}pG, m⁷GpppA_{2′OMe}pU, m⁷GpppC_{2′OMe}pA, m⁷GpppC_{2′OMe}pC, m⁷GpppC_{2′OMe}pG, m⁷GpppC_{2′OMe}pU, m⁷GpppG_{2′OMe}pA, m⁷GpppG_{2′OMe}pC, m⁷GpppG_{2′OMe}pG, m⁷GpppG_{2′OMe}pU, m⁷GpppU_{2′OMe}pA, m⁷GpppU_{2′OMe}pC, m⁷GpppU_{2′OMe}pG, and m⁷GpppU_{2′OMe}pU.

In some embodiments, a cap comprises m⁷GpppA_{2′OMe}pA. In some embodiments, a cap comprises m⁷GpppA_{2′OMe}pC. In some embodiments, a cap comprises m⁷GpppA_{2′OMe}pG. In some embodiments, a cap comprises m⁷GpppA_{2′OMe}pU. In some embodiments, a cap comprises m⁷GpppC_{2′OMe}pA. In some embodiments, a cap comprises m⁷GpppC_{2′OMe}pC. In some embodiments, a cap comprises m⁷GpppC_{2′OMe}pG. In some embodiments, a cap comprises m⁷GpppC_{2′OMe}pU. In some embodiments, a cap comprises m⁷GpppG_{2′OMe}pA. In some embodiments, a cap comprises m⁷GpppG_{2′OMe}pC. In some embodiments, a cap comprises m⁷GpppG_{2′OMe}pG. In some embodiments, a cap comprises m⁷GpppG_{2′OMe}pU. In some embodiments, a cap comprises m⁷GpppU_{2′OMe}pA. In some embodiments, a cap comprises m⁷GpppU_{2′OMe}pC. In some embodiments, a cap comprises m⁷GpppU_{2′OMe}pG. In some embodiments, a cap comprises m⁷GpppU_{2′OMe}pU.

In some embodiments, a cap comprises m⁷Gpppm⁶A_{2′OMe}pG. In some embodiments, a cap comprises m⁷Gpppe⁶A_{2′OMe}pG.

In some embodiments, a cap comprises GAG. In some embodiments, a cap comprises GCG. In some embodiments, a cap comprises GUG. In some embodiments, a cap comprises GGG.

In some embodiments, a cap comprises any one of the following structures:

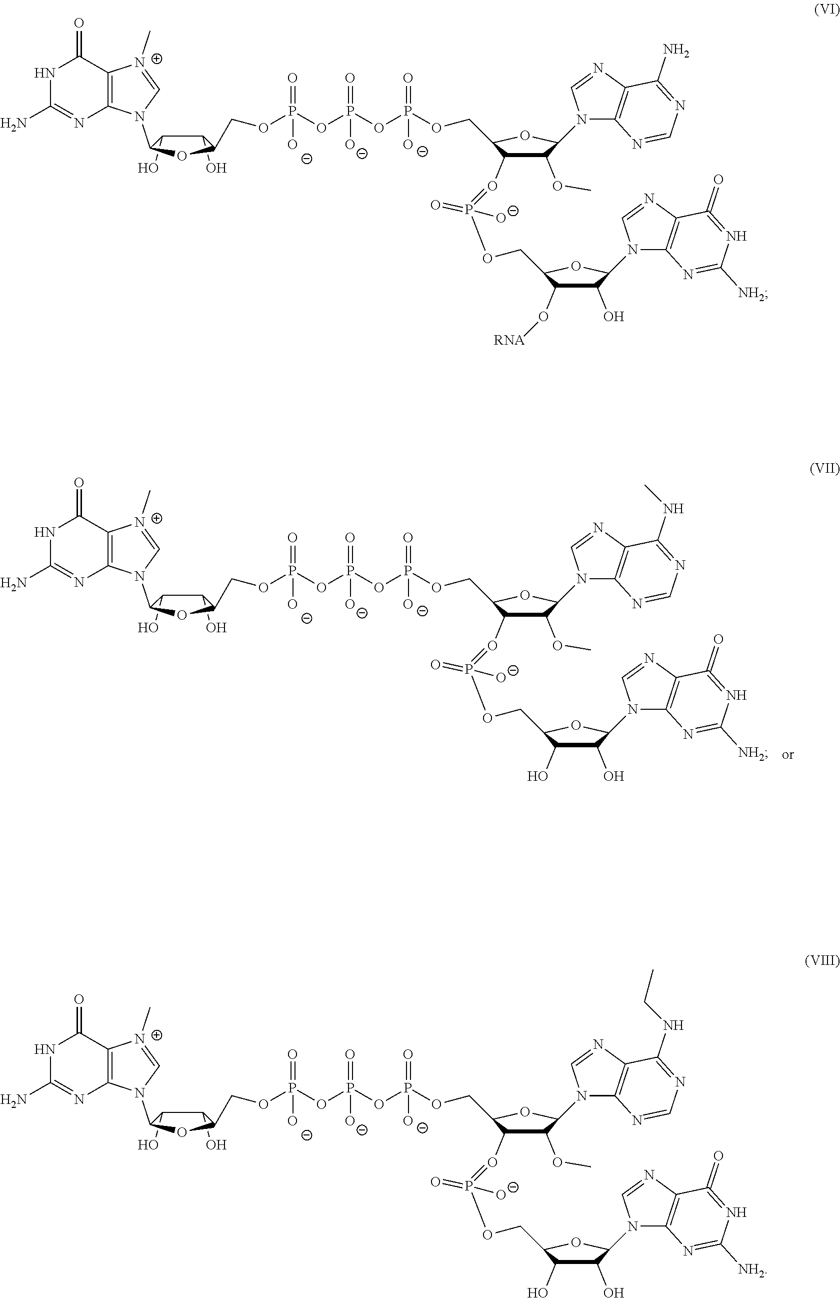

In some embodiments, the cap comprises ^{m7}GpppN₁N₂N₃, where N₁, N₂, and N₃ are optional (i.e., can be absent or one or more can be present) and are independently a natural, a modified, or an unnatural nucleoside base. In some embodiments, ^{m7}G is further methylated, e.g., at the 3′ position. In some embodiments, the ^{m7}G comprises an O-methyl at the 3′ position. In some embodiments N₁, N₂, and N₃ if present, optionally, are independently an adenine, a uracil, a guanidine, a thymine, or a cytosine. In some embodiments, one or more (or all) of N₁, N₂, and N₃, if present, are methylated, e.g., at the 2′ position. In some embodiments, one or more (or all) of N₁, N₂, and N₃, if present have an O-methyl at the 2′ position.

In some embodiments, the cap comprises the following structure:

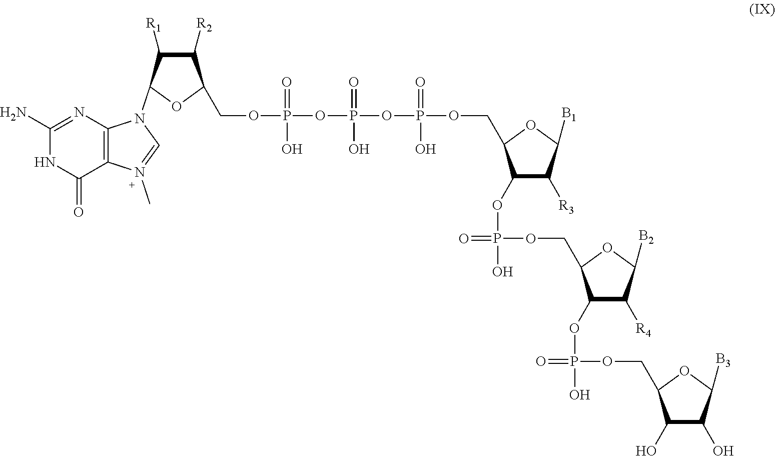

wherein B₁, B₂, and B₃ are independently a natural, a modified, or an unnatural nucleoside based; and R₁, R₂, R₃, and R₄ are independently OH or O-methyl. In some embodiments, R₃ is O-methyl and R₄ is OH. In some embodiments, R₃ and R₄ are O-methyl. In some embodiments, R₄ is O-methyl. In some embodiments, R₁ is OH, R₂ is OH, R₃ is O-methyl, and R₄ is OH. In some embodiments, R₁ is OH, R₂ is OH, R₃ is O-methyl, and R₄ is O-methyl. In some embodiments, at least one of R₁ and R₂ is O-methyl, R₃ is O-methyl, and R₄ is OH. In some embodiments, at least one of R₁ and R₂ is O-methyl, R₃ is O-methyl, and R₄ is O-methyl.

In some embodiments, B₁, B₃, and B₃ are natural nucleoside bases. In some embodiments, at least one of B₁, B₂, and B₃ is a modified or unnatural base. In some embodiments, at least one of B₁, B₂, and B₃ is N6-methyladenine. In some embodiments, B₁ is adenine, cytosine, thymine, or uracil. In some embodiments, B₁ is adenine, B₂ is uracil, and B₃ is adenine. In some embodiments, R₁ and R₂ are OH, R₃ and R₄ are O-methyl, B₁ is adenine, B₂ is uracil, and B₃ is adenine.

In some embodiments the cap comprises a sequence selected from the following sequences: GAAA, GACA, GAGA, GAUA, GCAA, GCCA, GCGA, GCUA, GGAA, GGCA, GGGA, GGUA, GUCA, and GUUA. In some embodiments the cap comprises a sequence selected from the following sequences: GAAG, GACG, GAGG, GAUG, GCAG, GCCG, GCGG, GCUG, GGAG, GGCG, GGGG, GGUG, GUCG, GUGG, and GUUG. In some embodiments the cap comprises a sequence selected from the following sequences: GAAU, GACU, GAGU, GAUU, GCAU, GCCU, GCGU, GCUU, GGAU, GGCU, GGGU, GGUU, GUAU, GUCU, GUGU, and GUUU. In some embodiments the cap comprises a sequence selected from the following sequences: GAAC, GACC, GAGC, GAUC, GCAC, GCCC, GCGC, GCUC, GGAC, GGCC, GGGC, GGUC, GUAC, GUCC, GUGC, and GUUC.

A cap, in some embodiments, comprises a sequence selected from the following sequences: m⁷G_{3′OMe}pppApApN, m⁷G_{3′OMe}pppApCpN, m⁷G_{3′OMe}pppApGpN, m⁷G_{3′OMe}pppApUpN, m⁷G_{3′OMe}pppCpApN, m⁷G_{3′OMe}pppCpCpN, m⁷G_{3′OMe}pppCpGpN, m⁷G_{3′OMe}pppCpUpN, m⁷G_{3′OMe}pppGpApN, m⁷G_{3′OMe}pppGpCpN, m⁷G_{3′OMe}pppGpGpN, m⁷G_{3′OMe}pppGpUpN, m⁷G_{3′OMe}pppUpApN, m⁷G_{3′OMe}pppUpCpN, m⁷G_{3′OMe}pppUpGpN, and m⁷G_{3′OMe}pppUpUpN, where N is a natural, a modified, or an unnatural nucleoside base.

A cap, in other embodiments, comprises a sequence selected from the following sequences: m⁷G_{3′OMe}pppA_{2′OMe}pApN, m⁷G_{3′OMe}pppA_{2′OMe}pCpN, m⁷G_{3′OMe}pppA_{2′OMe}pGpN, m⁷G_{3′OMe}pppA_{2′OMe}pUpN, m⁷G_{3′OMe}pppC_{2′OMe}pApN, m⁷G_{3′OMe}pppC_{2′OMe}pCpN, m⁷G_{3′OMe}pppC_{2′OMe}pGpN, m⁷G_{3′OMe}pppC_{2′OMe}pUpN, m⁷G_{3′OMe}pppG_{2′OMe}pApN, m⁷G_{3′OMe}pppG_{2′OMe}pCpN, m⁷G_{3′OMe}pppG_{2′OMe}pGpN, m⁷G_{3′OMe}pppG_{2′OMe}pUpN, m⁷G_{3′OMe}pppU_{2′OMe}pApN, m⁷G_{3′OMe}pppU_{2′OMe}pCpN, m⁷G_{3′OMe}pppU_{2′OMe}pGpN, and m⁷G_{3′OMe}pppU_{2′OMe}pUpN, where N is a natural, a modified, or an unnatural nucleoside base.

A cap, in still other embodiments, comprises a sequence selected from the following sequences: m⁷GpppA_{2′OMe}pApN, m⁷GpppA_{2′OMe}pCpN, m⁷GpppA_{2′OMe}pGpN, m⁷GpppA_{2′OMe}pUpN, m⁷GpppC_{2′OMe}pApN, m⁷GpppC_{2′OMe}pCpN, m⁷GpppC_{2′OMe}pGpN, m⁷GpppC_{2′OMe}pUpN, m⁷GpppG_{2′OMe}pApN, m⁷GpppG_{2′OMe}pCpN, m⁷GpppG_{2′OMe}pGpN, m⁷GpppG_{2′OMe}pUpN, m⁷GpppU_{2′OMe}pApN, m⁷GpppU_{2′OMe}pCpN, m⁷GpppU_{2′OMe}pGpN, and m⁷GpppU_{2′OMe}pUpN, where N is a natural, a modified, or an unnatural nucleoside base.

A cap, in other embodiments, comprises a sequence selected from the following sequences: m⁷G_{3′OMe}pppA_{2′OMe}pA_{2′OMe}pN, m⁷G_{3′OMe}pppA_{2′OMe}pC_{2′OMe}pN, m⁷G_{3′OMe}pppA_{2′OMe}pG_{2′OMe}pN, m⁷G_{3′OMe}pppA_{2′OMe}pU_{2′OMe}pN, m⁷G_{3′OMe}pppC_{2′OMe}pA_{2′OMe}pN, m⁷G_{3′OMe}pppC_{2′OMe}pC_{2′OMe}pN, m⁷G_{3′OMe}pppC_{2′OMe}pG_{2′OMe}pN, m⁷G_{3′OMe}pppC_{2′OMe}pU_{2′OMe}pN, m⁷G_{3′OMe}pppG_{2′OMe}pA_{2′OMe}pN, m⁷G_{3′OMe}pppG_{2′OMe}pC_{2′OMe}pN, m⁷G_{3′OMe}pppG_{2′OMe}pG_{2′OMe}pN, m⁷G_{3′OMe}pppG_{2′OMe}pU_{2′OMe}pN, m⁷G_{3′OMe}pppU_{2′OMe}pA_{2′OMe}pN, m⁷G_{3′OMe}pppU_{2′OMe}pC_{2′OMe}pN, m⁷G_{3′OMe}pppU_{2′OMe}pG_{2′OMe}pN, and m⁷G_{3′OMe}pppU_{2′OMe}pU_{2′OMe}pN, where N is a natural, a modified, or an unnatural nucleoside base.

A cap, in still other embodiments, comprises a sequence selected from the following sequences: m⁷GpppA_{2′OMe}pA_{2′OMe}pN, m⁷GpppA_{2′OMe}pC_{2′OMe}pN, m⁷GpppA_{2′OMe}pG_{2′OMe}pN, m⁷GpppA_{2′OMe}pU_{2′OMe}pN, m⁷GpppC_{2′OMe}pA_{2′OMe}pN, m⁷GpppC_{2′OMe}pC_{2′OMe}pN, m⁷GpppC_{2′OMe}pG_{2′OMe}pN, m⁷GpppC_{2′OMe}pU_{2′OMe}pN, m⁷GpppG_{2′OMe}pA_{2′OMe}pN, m⁷GpppG_{2′OMe}pC_{2′OMe}pN, m⁷GpppG_{2′OMe}pG_{2′OMe}pN, m⁷GpppG_{2′OMe}pU_{2′OMe}pN, m⁷GpppU_{2′OMe}pA_{2′OMe}pN, m⁷GpppU_{2′OMe}pC_{2′OMe}pN, m⁷GpppU_{2′OMe}pG_{2′OMe}pN, and m⁷GpppU_{2′OMe}pU_{2′OMe}pN, where N is a natural, a modified, or an unnatural nucleoside base.

In some embodiments, a cap comprises GGAG. In some embodiments, a cap comprises the following structure:

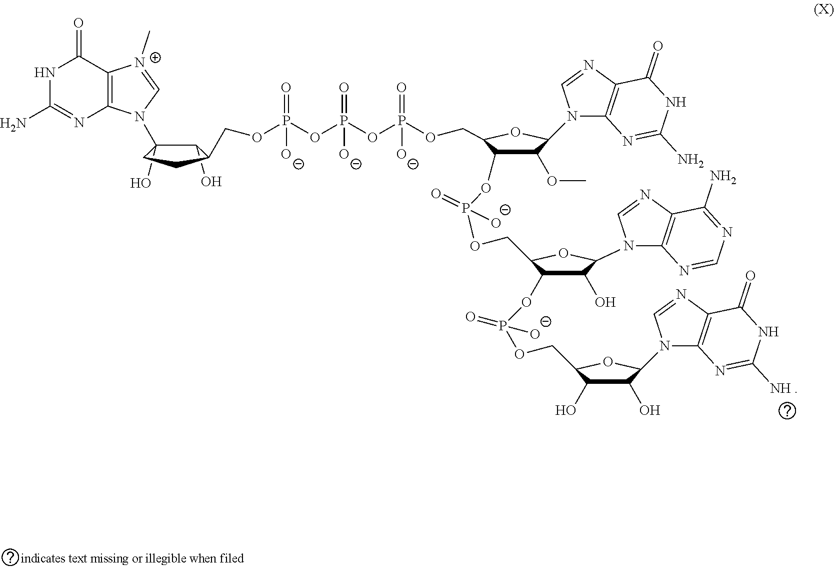

### Poly A Tails

In some embodiments, the polynucleotides of the present disclosure further comprise a poly-A tail. In further embodiments, terminal groups on the poly-A tail can be incorporated for stabilization. In other embodiments, a poly-A tail comprises des-3′ hydroxyl tails.

During RNA processing, a long chain of adenine nucleotides (poly-A tail) can be added to a polynucleotide such as an mRNA molecule to increase stability. Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long. In one embodiment, the poly-A tail is 100 nucleotides in length (SEQ ID NO: 121).

(SEQ ID NO: 121)
aaaaaaaaad aaaaaaaaad aaaaaaaaad aaaaaaaaad
aaaaaaaaad aaaaaaaaad aaaaaaaaad aaaaaaaaad
aaaaaaaaaa aaaaaaaaaa

PolyA tails can also be added after the construct is exported from the nucleus.

According to the present invention, terminal groups on the poly A tail can be incorporated for stabilization. Polynucleotides of the present invention can include des-3′ hydroxyl tails. They can also include structural moieties or 2′-Omethyl modifications as taught by Junjie Li, et al. (Current Biology, Vol. 15, 1501-1507, Aug. 23, 2005, the contents of which are incorporated herein by reference in its entirety).

The polynucleotides of the present invention can be designed to encode transcripts with alternative polyA tail structures including histone mRNA. According to Norbury, “Terminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs” (Norbury, “Cytoplasmic RNA: a case of the tail wagging the dog,” Nature Reviews Molecular Cell Biology; AOP, published online 29 Aug. 2013; doi:10.1038/nrm3645) the contents of which are incorporated herein by reference in its entirety.

Unique poly-A tail lengths provide certain advantages to the polynucleotides of the present invention. Generally, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present invention are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone (SEQ ID NO:51).

(SEQ ID NO: 51)
aaaaaaaaad aaaaaaaaad aaaaaaaaad aaaaaaaaad
aaaaaaaaad aaaaaaaaad aaaaaaaaad aaaaaaaaad
aaaaaaaaaa aaaaaaaaaa aaaaaaaaaa aaaaaaaaaa

In some embodiments, the poly-A tail is a mixed poly-A tail with intermittent non-adenosine residues (e.g., guanosine). In some embodiments, the poly-A tail is guanylated. In some embodiments, the mixed poly-A tail is a result of recruitment of one or more TENTs (e.g., TENT4A and/or TENT4B). Without wishing to be bound by theory, it is believed that in some embodiments the mixed poly-A tail can shield mRNA from rapid deadenylation.

In some embodiments, the poly-A tail comprises one or more non-adenosine residues. In some embodiments, the non-adenosine residue is guanosine. In some embodiments, the poly-A tail comprises 1-20, e.g., 1-15, 1-10, 1-5, 15-20, 10-20, 5-20, 2-15, 5-10, 1-5, 2-10, or 5-15, non-adenosine residues (e.g., guanosine). For example, the poly-A tail can comprise 1, 2, 3, 4, 5, 6. 7, 8, 9, 10, 11, 12, 13, 14, 15, 16, 17, 18, 19, 20, or more. non-adenosine residues (e.g., guanosine).

In some embodiments, at least 1%, e.g., at least 2%, 5%, 10%, 15%, 20%, or 25%, of the residues in the poly-A tail are non-adenosine residues (e.g., guanosine). In some embodiments, the poly-A tail is guanylated, e.g., comprising one or more guanosine residues.

In an embodiment, the poly-A tail comprising one or more non-adenosine residues is chemically synthesized.

### Start Codon Region

The invention also includes a polynucleotide that comprises both a start codon region and the polynucleotide described herein. In some embodiments, the polynucleotides of the present invention can have regions that are analogous to or function like a start codon region.

In some embodiments, the translation of a polynucleotide can initiate on a codon that is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG (see Touriol et al. Biology of the Cell 95 (2003) 169-178 and Matsuda and Mauro PLoS ONE, 2010 5:11; the contents of each of which are herein incorporated by reference in its entirety).

As a non-limiting example, the translation of a polynucleotide begins on the alternative start codon ACG. As another non-limiting example, polynucleotide translation begins on the alternative start codon CTG or CUG. As another non-limiting example, the translation of a polynucleotide begins on the alternative start codon GTG or GUG.

Nucleotides flanking a codon that initiates translation such as, but not limited to, a start codon or an alternative start codon, are known to affect the translation efficiency, the length and/or the structure of the polynucleotide. (See, e.g., Matsuda and Mauro PLoS ONE, 2010 5:11; the contents of which are herein incorporated by reference in its entirety). Masking any of the nucleotides flanking a codon that initiates translation can be used to alter the position of translation initiation, translation efficiency, length and/or structure of a polynucleotide.

In some embodiments, a masking agent can be used near the start codon or alternative start codon to mask or hide the codon to reduce the probability of translation initiation at the masked start codon or alternative start codon. Non-limiting examples of masking agents include antisense locked nucleic acids (LNA) polynucleotides and exon-junction complexes (EJCs) (See, e.g., Matsuda and Mauro describing masking agents LNA polynucleotides and EJCs (PLoS ONE, 2010 5:11); the contents of which are herein incorporated by reference in its entirety).

In another embodiment, a masking agent can be used to mask a start codon of a polynucleotide to increase the likelihood that translation will initiate on an alternative start codon. In some embodiments, a masking agent can be used to mask a first start codon or alternative start codon to increase the chance that translation will initiate on a start codon or alternative start codon downstream to the masked start codon or alternative start codon.

In some embodiments, a start codon or alternative start codon can be located within a perfect complement for a miRNA binding site. The perfect complement of a miRNA binding site can help control the translation, length and/or structure of the polynucleotide similar to a masking agent. As a non-limiting example, the start codon or alternative start codon can be located in the middle of a perfect complement for a miRNA binding site. The start codon or alternative start codon can be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide or twenty-first nucleotide.

In another embodiment, the start codon of a polynucleotide can be removed from the polynucleotide sequence to have the translation of the polynucleotide begin on a codon that is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence to have translation initiate on a downstream start codon or alternative start codon. The polynucleotide sequence where the start codon was removed can further comprise at least one masking agent for the downstream start codon and/or alternative start codons to control or attempt to control the initiation of translation, the length of the polynucleotide and/or the structure of the polynucleotide.

### Methods of Making Polynucleotides

The present disclosure also provides methods for making a polynucleotide disclosed herein or a complement thereof. In some aspects, a polynucleotide (e.g., an mRNA) disclosed herein can be constructed using in vitro transcription.

In other aspects, a polynucleotide (e.g., an mRNA) disclosed herein can be constructed by chemical synthesis using an oligonucleotide synthesizer. In other aspects, a polynucleotide (e.g., an mRNA) disclosed herein is made by using a host cell. In certain aspects, a polynucleotide (e.g., an mRNA) disclosed herein is made by one or more combination of the IVT, chemical synthesis, host cell expression, or any other methods known in the art.

Naturally occurring nucleosides, non-naturally occurring nucleosides, or combinations thereof, can totally or partially naturally replace occurring nucleosides present in the candidate nucleotide sequence and can be incorporated into a sequence-optimized nucleotide sequence (e.g., an mRNA) encoding a therapeutic payload or prophylactic payload. The resultant mRNAs can then be examined for their ability to produce protein and/or produce a therapeutic outcome.

While RNA can be made synthetically using methods well known in the art, in one embodiment an RNA transcript (e.g., mRNA transcript) is synthesized by contacting a DNA template with a RNA polymerase (e.g., a T7 RNA polymerase or a T7 RNA polymerase variant) under conditions that result in the production of RNA transcript.

In some aspects, the present disclosure provides methods of performing an IVT (in vitro transcription) reaction, comprising contacting a DNA template with the RNA polymerase (e.g., a T7 RNA polymerase, such as a T7 RNA polymerase variant) in the presence of nucleoside triphosphates and buffer under conditions that result in the production of RNA transcripts.

Other aspects of the present disclosure provide capping methods, e.g., co-transcriptional capping methods or other methods known in the art. In one embodiment, a capping method comprises reacting a polynucleotide template with a T7 RNA polymerase variant, nucleoside triphosphates, and a cap analog under in vitro transcription reaction conditions to produce RNA transcript.

IVT conditions typically require a purified linear DNA template containing a promoter, nucleoside triphosphates, a buffer system that includes dithiothreitol (DTT) and magnesium ions, and a RNA polymerase. The exact conditions used in the transcription reaction depend on the amount of RNA needed for a specific application. Typical IVT reactions are performed by incubating a DNA template with a RNA polymerase and nucleoside triphosphates, including GTP, ATP, CTP, and UTP (or nucleotide analogs) in a transcription buffer. A RNA transcript having a 5′ terminal guanosine triphosphate is produced from this reaction.

A deoxyribonucleic acid (DNA) is simply a nucleic acid template for RNA polymerase. A DNA template may include a polynucleotide encoding a polypeptide of interest (e.g., an antigenic polypeptide). A DNA template, in some embodiments, includes a RNA polymerase promoter (e.g., a T7 RNA polymerase promoter) located 5′ from and operably linked to polynucleotide encoding a polypeptide of interest. A DNA template may also include a nucleotide sequence encoding a polyadenylation (polyA) tail located at the 3′ end of the gene of interest.

Polypeptides of interest include, but are not limited to, biologics, antibodies, antigens (vaccines), and therapeutic proteins. The term “protein” encompasses peptides.

A RNA transcript, in some embodiments, is the product of an IVT reaction and, as will be understood by one of ordinary skill in the art, the DNA template for making an RNA molecule is known based on base complementarity. A RNA transcript, in some embodiments, is a messenger RNA (mRNA) that includes a nucleotide sequence encoding a polypeptide of interest linked to a polyA tail. In some embodiments, the mRNA is modified mRNA (mmRNA), which includes at least one modified nucleotide.

A nucleotide includes a nitrogenous base, a five-carbon sugar (ribose or deoxyribose), and at least one phosphate group. Nucleotides include nucleoside monophosphates, nucleoside diphosphates, and nucleoside triphosphates. A nucleoside monophosphate (NMP) includes a nucleobase linked to a ribose and a single phosphate; a nucleoside diphosphate (NDP) includes a nucleobase linked to a ribose and two phosphates; and a nucleoside triphosphate (NTP) includes a nucleobase linked to a ribose and three phosphates. Nucleotide analogs are compounds that have the general structure of a nucleotide or are structurally similar to a nucleotide. Nucleotide analogs, for example, include an analog of the nucleobase, an analog of the sugar and/or an analog of the phosphate group(s) of a nucleotide.

A nucleoside includes a nitrogenous base and a 5-carbon sugar. Thus, a nucleoside plus a phosphate group yields a nucleotide. Nucleoside analogs are compounds that have the general structure of a nucleoside or are structurally similar to a nucleoside. Nucleoside analogs, for example, include an analog of the nucleobase and/or an analog of the sugar of a nucleoside.

It should be understood that the term “nucleotide” includes naturally-occurring nucleotides, synthetic nucleotides and modified nucleotides, unless indicated otherwise. Examples of naturally-occurring nucleotides used for the production of RNA, e.g., in an IVT reaction, as provided herein include adenosine triphosphate (ATP), guanosine triphosphate (GTP), cytidine triphosphate (CTP), uridine triphosphate (UTP), and 5-methyluridine triphosphate (m⁵UTP). In some embodiments, adenosine diphosphate (ADP), guanosine diphosphate (GDP), cytidine diphosphate (CDP), and/or uridine diphosphate (UDP) are used.

Examples of nucleotide analogs include, but are not limited to, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (vaccinia or ligase), a nucleotide labeled with a functional group to facilitate ligation/conjugation of cap or 5′ moiety (IRES), a nucleotide labeled with a 5′ PO₄ to facilitate ligation of cap or 5′ moiety, or a nucleotide labeled with a functional group/protecting group that can be chemically or enzymatically cleaved. Examples of antiviral nucleotide/nucleoside analogs include, but are not limited, to Ganciclovir, Entecavir, Telbivudine, Vidarabine and Cidofovir.

Modified nucleotides may include modified nucleobases. For example, a RNA transcript (e.g., mRNA transcript) of the present disclosure may include a modified nucleobase selected from pseudouridine (ψ), 1-methylpseudouridine (m1ψ), 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, a RNA transcript (e.g., mRNA transcript) includes a combination of at least two (e.g., 2, 3, 4 or more) of the foregoing modified nucleobases.

The nucleoside triphosphates (NTPs) as provided herein may comprise unmodified or modified ATP, modified or unmodified UTP, modified or unmodified GTP, and/or modified or unmodified CTP. In some embodiments, NTPs of an IVT reaction comprise unmodified ATP. In some embodiments, NTPs of an IVT reaction comprise modified ATP. In some embodiments, NTPs of an IVT reaction comprise unmodified UTP. In some embodiments, NTPs of an IVT reaction comprise modified UTP. In some embodiments, NTPs of an IVT reaction comprise unmodified GTP. In some embodiments, NTPs of an IVT reaction comprise modified GTP. In some embodiments, NTPs of an IVT reaction comprise unmodified CTP. In some embodiments, NTPs of an IVT reaction comprise modified CTP.

The concentration of nucleoside triphosphates and cap analog present in an IVT reaction may vary. In some embodiments, NTPs and cap analog are present in the reaction at equimolar concentrations. In some embodiments, the molar ratio of cap analog (e.g., trinucleotide cap) to nucleoside triphosphates in the reaction is greater than 1:1. For example, the molar ratio of cap analog to nucleoside triphosphates in the reaction may be 2:1, 3:1, 4:1, 5:1, 6:1, 7:1, 8:1, 9:1, 10:1, 15:1, 20:1, 25:1, 50:1, or 100:1. In some embodiments, the molar ratio of cap analog (e.g., trinucleotide cap) to nucleoside triphosphates in the reaction is less than 1:1. For example, the molar ratio of cap analog (e.g., trinucleotide cap) to nucleoside triphosphates in the reaction may be 1:2, 1:3, 1:4, 1:5, 1:6, 1:7, 1:8, 1:9, 1:10, 1:15, 1:20, 1:25, 1:50, or 1:100.

The composition of NTPs in an IVT reaction may also vary. For example, ATP may be used in excess of GTP, CTP and UTP. As a non-limiting example, an IVT reaction may include 7.5 millimolar GTP, 7.5 millimolar CTP, 7.5 millimolar UTP, and 3.75 millimolar ATP. The same IVT reaction may include 3.75 millimolar cap analog (e.g., trinucleotide cap). In some embodiments, the molar ratio of G:C:U:A:cap is 1:1:1:0.5:0.5. In some embodiments, the molar ratio of G:C:U:A:cap is 1:1:0.5:1:0.5. In some embodiments, the molar ratio of G:C:U:A:cap is 1:0.5:1:1:0.5. In some embodiments, the molar ratio of G:C:U:A:cap is 0.5:1:1:1:0.5.

In some embodiments, a RNA transcript (e.g., mRNA transcript) includes a modified nucleobase selected from pseudouridine (ψ), 1-methylpseudouridine (m¹ψ), 5-methoxyuridine (mo⁵U), 5-methylcytidine (m⁵C), α-thio-guanosine and α-thio-adenosine. In some embodiments, a RNA transcript (e.g., mRNA transcript) includes a combination of at least two (e.g., 2, 3, 4 or more) of the foregoing modified nucleobases.

In some embodiments, a RNA transcript (e.g., mRNA transcript) includes pseudouridine (ψ). In some embodiments, a RNA transcript (e.g., mRNA transcript) includes 1-methylpseudouridine (m¹ψ). In some embodiments, a RNA transcript (e.g., mRNA transcript) includes 5-methoxyuridine (mo⁵U). In some embodiments, a RNA transcript (e.g., mRNA transcript) includes 5-methylcytidine (m⁵C). In some embodiments, a RNA transcript (e.g., mRNA transcript) includes α-thio-guanosine. In some embodiments, a RNA transcript (e.g., mRNA transcript) includes α-thio-adenosine.

In some embodiments, the polynucleotide (e.g., RNA polynucleotide, such as mRNA polynucleotide) is uniformly modified (e.g., fully modified, modified throughout the entire sequence) for a particular modification. For example, a polynucleotide can be uniformly modified with 1-methylpseudouridine (m¹ψ), meaning that all uridine residues in the mRNA sequence are replaced with 1-methylpseudouridine (m¹ψ). Similarly, a polynucleotide can be uniformly modified for any type of nucleoside residue present in the sequence by replacement with a modified residue such as any of those set forth above. Alternatively, the polynucleotide (e.g., RNA polynucleotide, such as mRNA polynucleotide) may not be uniformly modified (e.g., partially modified, part of the sequence is modified). Each possibility represents a separate embodiment of the present invention.

In some embodiments, the buffer system contains tris. The concentration of tris used in an IVT reaction, for example, may be at least 10 mM, at least 20 mM, at least 30 mM, at least 40 mM, at least 50 mM, at least 60 mM, at least 70 mM, at least 80 mM, at least 90 mM, at least 100 mM or at least 110 mM phosphate. In some embodiments, the concentration of phosphate is 20-60 mM or 10-100 mM.

In some embodiments, the buffer system contains dithiothreitol (DTT). The concentration of DTT used in an IVT reaction, for example, may be at least 1 mM, at least 5 mM, or at least 50 mM. In some embodiments, the concentration of DTT used in an IVT reaction is 1-50 mM or 5-50 mM. In some embodiments, the concentration of DTT used in an IVT reaction is 5 mM.

In some embodiments, the buffer system contains magnesium. In some embodiments, the molar ratio of NTP to magnesium ions (Mg²⁺; e.g., MgCl₂) present in an IVT reaction is 1:1 to 1:5. For example, the molar ratio of NTP to magnesium ions may be 1:1, 1:2, 1:3, 1:4 or 1:5.

In some embodiments, the molar ratio of NTP plus cap analog (e.g., trinucleotide cap, such as GAG) to magnesium ions (Mg²⁺; e.g., MgCl₂) present in an IVT reaction is 1:1 to 1:5. For example, the molar ratio of NTP+trinucleotide cap (e.g., GAG) to magnesium ions may be 1:1, 1:2, 1:3, 1:4 or 1:5.

In some embodiments, the buffer system contains Tris-HCl, spermidine (e.g., at a concentration of 1-30 mM), TRITON© X-100 (polyethylene glycol p-(1,1,3,3-tetramethylbutyl)-phenyl ether) and/or polyethylene glycol (PEG).

The addition of nucleoside triphosphates (NTPs) to the 3′ end of a growing RNA strand is catalyzed by a polymerase, such as T7 RNA polymerase, for example, any one or more of the T7 RNA polymerase variants (e.g., G47A) of the present disclosure. In some embodiments, the RNA polymerase (e.g., T7 RNA polymerase variant) is present in a reaction (e.g., an IVT reaction) at a concentration of 0.01 mg/ml to 1 mg/ml. For example, the RNA polymerase may be present in a reaction at a concentration of 0.01 mg/mL, 0.05 mg/ml, 0.1 mg/ml, 0.5 mg/ml or 1.0 mg/ml.

In some embodiments, the polynucleotide of the present disclosure is an IVT polynucleotide. Traditionally, the basic components of an mRNA molecule include at least a coding region, a 5′ UTR, a 3′ UTR, a 5′ cap and a poly-A tail. The IVT polynucleotides of the present disclosure can function as mRNA but are distinguished from wild-type mRNA in their functional and/or structural design features which serve, e.g., to overcome existing problems of effective polypeptide production using nucleic-acid based therapeutics.

The primary construct of an IVT polynucleotide comprises a first region of linked nucleotides that is flanked by a first flanking region and a second flaking region. This first region can include, but is not limited to, the encoded therapeutic payload or prophylactic payload. The first flanking region can include a sequence of linked nucleosides which function as a 5′ untranslated region (UTR) such as the 5′ UTR of any of the nucleic acids encoding the native 5′ UTR of the polypeptide or a non-native 5′ UTR such as, but not limited to, a heterologous 5′ UTR or a synthetic 5′ UTR. The IVT encoding a therapeutic payload or prophylactic payload can comprise at its 5 terminus a signal sequence region encoding one or more signal sequences. The flanking region can comprise a region of linked nucleotides comprising one or more complete or incomplete 5′ UTRs sequences. The flanking region can also comprise a 5′ terminal cap. The second flanking region can comprise a region of linked nucleotides comprising one or more complete or incomplete 3′ UTRs which can encode the native 3′ UTR of a therapeutic payload or prophylactic payload, or a non-native 3′ UTR such as, but not limited to, a heterologous 3′ UTR or a synthetic 3′ UTR. The flanking region can also comprise a 3′ tailing sequence. The 3′ tailing sequence can be, but is not limited to, a polyA tail, a polyA-G quartet and/or a stem loop sequence.

Additional and exemplary features of IVT polynucleotide architecture and methods of making a polynucleotide are disclosed in International PCT application WO 2017/201325, filed on 18 May 2017, the entire contents of which are hereby incorporated by reference.

### Purification

In other aspects, a polynucleotide (e.g., an mRNA) disclosed herein can be purified. Purification of the polynucleotides (e.g., mRNA) described herein can include, but is not limited to, polynucleotide clean-up, quality assurance and quality control. Clean-up can be performed by methods known in the arts such as, but not limited to, AGENCOURT® beads (Beckman Coulter Genomics, Danvers, Mass.), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a polynucleotide such as a “purified polynucleotide” refers to one that is separated from at least one contaminant. As used herein, a “contaminant” is any substance which makes another unfit, impure or inferior. Thus, a purified polynucleotide (e.g., DNA and RNA) is present in a form or setting different from that in which it is found in nature, or a form or setting different from that which existed prior to subjecting it to a treatment or purification method.

In some embodiments, purification of a polynucleotide (e.g., mRNA) of the disclosure removes impurities that can reduce or remove an unwanted immune response, e.g., reducing cytokine activity.

In some embodiments, the polynucleotide (e.g., mRNA) of the disclosure is purified prior to administration using column chromatography (e.g., strong anion exchange HPLC, weak anion exchange HPLC, reverse phase HPLC (RP-HPLC), and hydrophobic interaction HPLC (HIC-HPLC), or (LCMS)). In some embodiments, a column chromatography (e.g., strong anion exchange HPLC, weak anion exchange HPLC, reverse phase HPLC (RP-HPLC), and hydrophobic interaction HPLC (HIC-HPLC), or (LCMS)) purified polynucleotide, which encodes a therapeutic payload or prophylactic payload disclosed herein increases expression of the therapeutic payload or prophylactic payload, compared to polynucleotides encoding the therapeutic payload or prophylactic payload, purified by a different purification method.

In some embodiments, a column chromatography (e.g., strong anion exchange HPLC, weak anion exchange HPLC, reverse phase HPLC (RP-HPLC), and hydrophobic interaction HPLC (HIC-HPLC), or (LCMS)) purified polynucleotide encodes a therapeutic payload or prophylactic payload. In some embodiments, the purified polynucleotide encodes a therapeutic payload or prophylactic payload.

In some embodiments, the purified polynucleotide is at least about 80% pure, at least about 85% pure, at least about 90% pure, at least about 95% pure, at least about 96% pure, at least about 97% pure, at least about 98% pure, at least about 99% pure, or about 100% pure.

A quality assurance and/or quality control check can be conducted using methods such as, but not limited to, gel electrophoresis, UV absorbance, or analytical HPLC.

In another embodiment, the polynucleotides can be sequenced by methods including, but not limited to reverse-transcriptase-PCR.

### Chemical Modifications of Polynucleotides

As described above, modified nucleosides and nucleotides of a nucleic acid (e.g., RNA nucleic acids, such as mRNA nucleic acids) may be included in a polynucleotide of the invention. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise N1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, an RNA nucleic acid of the disclosure comprises N1-methyl-pseudouridine (m1ψ) substitutions at one or more or all uridine positions of the nucleic acid.

In some embodiments, an RNA nucleic acid of the disclosure comprises N1-methyl-pseudouridine (m1ψ) substitutions at one or more or all uridine positions of the nucleic acid and 5-methyl cytidine substitutions at one or more or all cytidine positions of the nucleic acid.

In some embodiments, an RNA nucleic acid of the disclosure comprises pseudouridine (ψ) substitutions at one or more or all uridine positions of the nucleic acid.

In some embodiments, an RNA nucleic acid of the disclosure comprises pseudouridine (ψ) substitutions at one or more or all uridine positions of the nucleic acid and 5-methyl cytidine substitutions at one or more or all cytidine positions of the nucleic acid.

In some embodiments, an RNA nucleic acid of the disclosure comprises uridine at one or more or all uridine positions of the nucleic acid.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with N1-methyl-pseudouridine, meaning that all uridine residues in the mRNA sequence are replaced with N1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the polyA tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

Sequence Optimization and Methods Thereof In some embodiments, a polynucleotide of the disclosure comprises a sequence-optimized nucleotide sequence encoding a polypeptide disclosed herein, e.g., a polynucleotide encoding a therapeutic payload or prophylactic payload. In some embodiments, the polynucleotide of the disclosure comprises an open reading frame (ORF) encoding a therapeutic payload or prophylactic payload, wherein the ORF has been sequence optimized.

The sequence-optimized nucleotide sequences disclosed herein are distinct from the corresponding wild type nucleotide acid sequences and from other known sequence-optimized nucleotide sequences, e.g., these sequence-optimized nucleic acids have unique compositional characteristics.

In some embodiments, the percentage of uracil or thymine nucleobases in a sequence-optimized nucleotide sequence is modified (e.g., reduced) with respect to the percentage of uracil or thymine nucleobases in the reference wild-type nucleotide sequence. Such a sequence is referred to as a uracil-modified or thymine-modified sequence. The percentage of uracil or thymine content in a nucleotide sequence can be determined by dividing the number of uracils or thymines in a sequence by the total number of nucleotides and multiplying by 100. In some embodiments, the sequence-optimized nucleotide sequence has a lower uracil or thymine content than the uracil or thymine content in the reference wild-type sequence. In some embodiments, the uracil or thymine content in a sequence-optimized nucleotide sequence of the disclosure is greater than the uracil or thymine content in the reference wild-type sequence and still maintain beneficial effects, e.g., increased expression and/or signaling response when compared to the reference wild-type sequence.

In some embodiments, the optimized sequences of the present disclosure contain unique ranges of uracils or thymine (if DNA) in the sequence. The uracil or thymine content of the optimized sequences can be expressed in various ways, e.g., uracil or thymine content of optimized sequences relative to the theoretical minimum (% UTM or % TTM), relative to the wild-type (% UWT or % TWT), and relative to the total nucleotide content (% UTL or % TTL). For DNA it is recognized that thymine (T) is present instead of uracil (U), and one would substitute T where U appears. For RNA it is recognized that uracil (U) is present instead of thymine (T). One of skill in the art could readily obtain an RNA sequence when the DNA sequence is provided by substituting thymine in the DNA sequence to uracil. Thus, all the disclosures related to, e.g., % UTM, % UWT, or % UTL, with respect to RNA are equally applicable to % TTM, % TWT, or % TTL with respect to DNA.

Uracil- or thymine-content relative to the uracil or thymine theoretical minimum, refers to a parameter determined by dividing the number of uracils or thymines in a sequence-optimized nucleotide sequence by the total number of uracils or thymines in a hypothetical nucleotide sequence in which all the codons in the hypothetical sequence are replaced with synonymous codons having the lowest possible uracil or thymine content and multiplying by 100. This parameter is abbreviated herein as % UTM or % TTM.

In some embodiments, a uracil-modified sequence of the disclosure has a reduced number of consecutive uracils with respect to the corresponding wild-type nucleic acid sequence.

For example, two consecutive leucines can be encoded by the sequence CUUUUG, which includes a four-uracil cluster. Such a subsequence can be substituted, e.g., with CUGCUC, which removes the uracil cluster. Phenylalanine can be encoded by UUC or UUU. Thus, even if phenylalanines encoded by UUU are replaced by UUC, the synonymous codon still contains a uracil pair (UU). Accordingly, the number of phenylalanines in a sequence establishes a minimum number of uracil pairs (UU) that cannot be eliminated without altering the number of phenylalanines in the encoded polypeptide.

In some embodiments, a uracil-modified sequence of the disclosure has a reduced number of uracil triplets (UUU) with respect to the wild-type nucleic acid sequence. In some embodiments, a uracil-modified sequence has a reduced number of uracil pairs (UU) with respect to the number of uracil pairs (UU) in the wild-type nucleic acid sequence. In some embodiments, a uracil-modified sequence of the disclosure has a number of uracil pairs (UU) corresponding to the minimum possible number of uracil pairs (UU) in the wild-type nucleic acid sequence.

The phrase “uracil pairs (UU) relative to the uracil pairs (UU) in the wild type nucleic acid sequence,” refers to a parameter determined by dividing the number of uracil pairs (UU) in a sequence-optimized nucleotide sequence by the total number of uracil pairs (UU) in the corresponding wild-type nucleotide sequence and multiplying by 100. This parameter is abbreviated herein as % UUwt. In some embodiments, a uracil-modified sequence has a % UUwt between below 100%.

In some embodiments, the polynucleotide of the disclosure comprises a uracil-modified sequence. In some embodiments, the uracil-modified sequence comprises at least one chemically modified nucleobase, e.g., 5-methoxyuracil. In some embodiments, at least 95% of a nucleobase (e.g., uracil) in a uracil-modified sequence of the disclosure are modified nucleobases. In some embodiments, at least 95% of uracil in a uracil-modified sequence is 5-methoxyuracil.

In some embodiments, a polynucleotide of the disclosure is sequence optimized.

A sequence optimized nucleotide sequence (nucleotide sequence is also referred to as “nucleic acid” herein) comprises at least one codon modification with respect to a reference sequence (e.g., a wild-type sequence encoding a therapeutic payload or prophylactic payload). Thus, in a sequence optimized nucleic acid, at least one codon is different from a corresponding codon in a reference sequence (e.g., a wild-type sequence).

In general, sequence optimized nucleic acids are generated by at least a step comprising substituting codons in a reference sequence with synonymous codons (i.e., codons that encode the same amino acid). Such substitutions can be effected, for example, by applying a codon substitution map (i.e., a table providing the codons that will encode each amino acid in the codon optimized sequence), or by applying a set of rules (e.g., if glycine is next to neutral amino acid, glycine would be encoded by a certain codon, but if it is next to a polar amino acid, it would be encoded by another codon). In addition to codon substitutions (i.e., “codon optimization”) the sequence optimization methods disclosed herein comprise additional optimization steps which are not strictly directed to codon optimization such as the removal of deleterious motifs (destabilizing motif substitution). Compositions and formulations comprising these sequence-optimized nucleic acids (e.g., an RNA, e.g., an mRNA) can be administered to a subject in need thereof to facilitate in vivo expression of functionally active encoding a therapeutic payload or prophylactic payload.

Additional and exemplary methods of sequence optimization are disclosed in International PCT application WO 2017/201325, filed on 18 May 2017, the entire contents of which are hereby incorporated by reference.

### Lipid Content of LNPs

As set forth above, with respect to lipids, LNPs for use as delivery vehicles disclosed herein comprise an (i) ionizable lipid; (ii) sterol or other structural lipid; (iii) a non-cationic helper lipid or phospholipid; and, optionally a (iv) PEG lipid. These categories of lipids are set forth in more detail below.

In some embodiments, nucleic acids of the invention are formulated as lipid nanoparticle (LNP) compositions. Lipid nanoparticles typically comprise amino lipid, phospholipid, structural lipid and PEG lipid components along with the nucleic acid cargo of interest. The lipid nanoparticles of the invention can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575; PCT/US2016/069491; PCT/US2016/069493; and PCT/US2014/66242, all of which are incorporated by reference herein in their entirety.

In some embodiments, the lipid nanoparticle comprises a molar ratio of 20-60% amino lipid relative to the other lipid components. For example, the lipid nanoparticle may comprise a molar ratio of 20-50%, 20-40%, 20-30%, 30-60%, 30-50%, 30-40%, 40-60%, 40-50%, or 50-60% amino lipid. In some embodiments, the lipid nanoparticle comprises a molar ratio of 20%, 30%, 40%, 50, or 60% amino lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%. 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-lipid.

In some embodiments, the lipid nanoparticle comprises a molar ratio of 20-60% amino lipid, 5-25% phospholipid, 25-55% structural lipid, and 0.5-15% PEG lipid.

In some embodiments, the lipid nanoparticle comprises a molar ratio of 20-60% amino lipid, 5-30% phospholipid, 10-55% structural lipid, and 0.5-15% PEG lipid.

### Amino Lipids

In some aspects, the amino lipids of the present disclosure may be one or more of compounds of Formula (I):

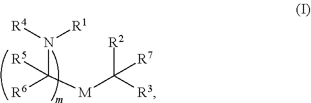

or their N-oxides, or salts or isomers thereof, wherein:

R¹ is selected from the group consisting of C₅₋₃₀ alkyl, C₅₋₂₀ alkenyl, —R*YR″, —YR″, and —R″M′R′;

R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;

R⁴ is selected from the group consisting of hydrogen, a C₃₋₆ carbocycle, —(CH₂)ₙQ, —(CH₂)ₙCHQR,

—CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, and each n is independently selected from 1, 2, 3, 4, and 5;

each R⁵ is independently selected from the group consisting of C₁₋₃ alkyl, C₂₋₃ alkenyl, and H;

each R⁶ is independently selected from the group consisting of C₁₋₃ alkyl, C₂₋₃ alkenyl, and H;

M and M′ are independently selected

from —C(O)O—, —OC(O)—, —OC(O)-M″-C(O)O—, —C(O)N(R′)—, —N(R′)C(O)—, —C(O)—, —C(S)—, —C(S) S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R⁷ is selected from the group consisting of C₁₋₃ alkyl, C₂₋₃ alkenyl, and H;

R⁸ is selected from the group consisting of C₃₋₆ carbocycle and heterocycle;

R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13; and wherein when R⁴ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In certain embodiments, a subset of compounds of Formula (I) includes those of Formula (IA):

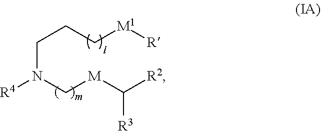

or its N-oxide, or a salt or isomer thereof, wherein 1 is selected from 1, 2, 3, 4, and 5; m is selected from 5, 6, 7, 8, and 9; M¹ is a bond or M′; R⁴ is hydrogen, unsubstituted C₁₋₃ alkyl, or —(CH₂)ₙQ, in which Q is

OH, —NHC(S)N(R)₂, —NHC(O)N(R)₂, —N(R)C(O)R, —N(R)S(O)₂R, —N(R)R⁸, —NHC(═NR⁹)N(R)₂, —NHC(═CHR⁹)N(R)₂, —OC(O)N(R)₂, —N(R)C(O)OR, heteroaryl or heterocycloalkyl; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (IB).

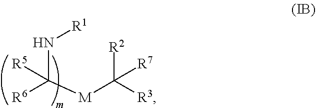

or its N-oxide, or a salt or isomer thereof in which all variables are as defined herein. For example, m is selected from 5, 6, 7, 8, and 9; R⁴ is hydrogen, unsubstituted C₁₋₃ alkyl, or —(CH₂)ₙQ, in which Q is
OH, —NHC(S)N(R)₂, —NHC(O)N(R)₂, —N(R)C(O)R, —N(R)S(O)₂R, —N(R)R⁸, —NHC(═NR⁹)N(R)₂, —NHC(═CHR⁹)N(R)₂, —OC(O)N(R)₂, —N(R)C(O)OR, heteroaryl or heterocycloalkyl; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl. For example, m is 5, 7, or 9. For example, Q is
OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (IC):

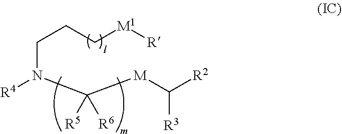

or its N-oxide, or a salt or isomer thereof in which all variables are as defined herein. For example, R′ is selected from the group consisting of branched C₁₋₁₈ alkyl and branched C₂₋₁₈ alkenyl; R² and R³ are each independently selected from the group consisting of C₁₋₁₄ alkyl and C₂₋₁₄ alkenyl; R⁴ is selected from the group consisting of —(CH₂)ₙOH, wherein n is selected from the group consisting of 1, 2, 3, 4, and 5, and

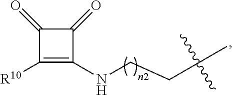

wherein

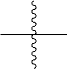

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10; each R⁵ is independently selected from the group consisting of C₁₋₃ alkyl, C₂₋₃ alkenyl, and H; each R⁶ is independently selected from the group consisting of C₁₋₃ alkyl, C₂₋₃ alkenyl, and H; M and M¹ are each independently selected from the group consisting of —C(O)O— and —OC(O)—; 1 is selected from the group consisting of 1, 2, 3, 4, and 5; and m is selected from the group consisting of 5, 6, 7, 8, 9, 10, 11, 12, and 13.

In some embodiments of the compounds of Formula (IC), R′ is

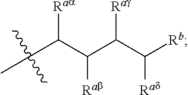

wherein

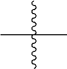

denotes a point of attachment; wherein R^{aα}, R^{aβ}, R^{aγ}, and R^{aδ} are each independently selected from the group consisting of H, C₂₋₁₂ alkyl, and C₂₋₁₂ alkenyl and R^{b} is a C₁₋₁₂ alkyl or C₂₋₁₂ alkenyl. In some embodiments of the compounds of Formula (IC), R′ is

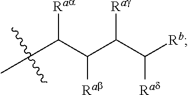

wherein

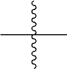

denotes a point of attachment; wherein R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R^{b} is a C₁₋₁₂ alkyl; 1 is 5; and m is 7. In some embodiments of Formula (IC), R′ is

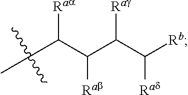

wherein

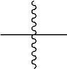

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of the compounds of Formula (IC), R′ is

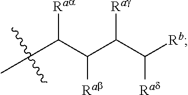

wherein

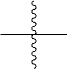

denotes a point of attachment; R^{aα} is C₂₋₁₂ alkyl; R^{aβ}, R^{aγ}, and R^{aδ} are each H; R² and R³ are each C₁₋₁₄ alkyl; R⁴ is

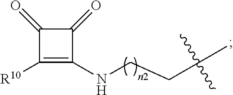

R¹⁰ NH(C₁₋₆ alkyl); n2 is 2; R⁵ is H; each R⁶ is H; M and M′ are each —C(O)O—; R′ is a C₁₋₁₂ alkyl; 1 is 5; and m is 7.

In some embodiments of the compounds of Formula (IC), R′ is

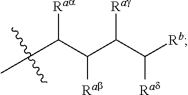

wherein

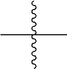

denotes a point of attachment; R^{aα}, R^{aβ}, and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments of the compounds of Formula (IC), R′ is

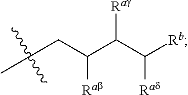

wherein

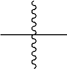

denotes a point of attachment; wherein R^{aβ}, R^{aγ}, and R^{aδ} are each independently selected from the group consisting of H, C₂₋₁₂ alkyl, and C₂₋₁₂ alkenyl and R^{b} is a C₁₋₁₂ alkyl or C₂₋₁₂ alkenyl. In some embodiments of the compounds of Formula (IC), R′ is

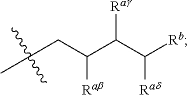

wherein R^{aβ}, R^{aγ}, and R^{aδ} are each independently selected from the group consisting of H, C₂₋₁₂ alkyl, and C₂₋₁₂ alkenyl; R² and R³ are each independently selected from the group consisting of C₁₋₁₄ alkyl and C₂₋₁₄ alkenyl; R⁴ is selected from the group consisting of —(CH₂)ₙOH wherein n is selected from the group consisting of 1, 2, 3, 4, and 5, and

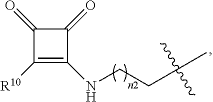

wherein

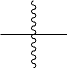

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10; each R⁵ is independently selected from the group consisting of C₁₋₃ alkyl, C₂₋₃ alkenyl, and H; each R⁶ is independently selected from the group consisting of C₁₋₃ alkyl, C₂₋₃ alkenyl, and H; M and M′ are each independently selected from the group consisting of —C(O)O— and —OC(O)—; R^{b} is a C₁₋₁₂ alkyl or C₂₋₁₂ alkenyl; l is selected from the group consisting of 1, 2, 3, 4, and 5; and m is selected from the group consisting of 5, 6, 7, 8, 9, 10, 11, 12, and 13.

In some embodiments of the compounds of Formula (IC), R′ is

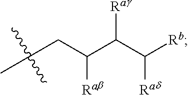

wherein

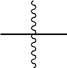

denotes a point of attachment; wherein R^{aα}, R^{aβ}, R^{aγ}, and R^{aδ} are each independently selected from the group consisting of H, C₂₋₁₂ alkyl, and C₂₋₁₂ alkenyl; R² and R³ are each independently selected from the group consisting of C₁₋₁₄ alkyl and C₂₋₁₄ alkenyl; R⁴ is —(CH₂)ₙOH, wherein n is selected from the group consisting of 1, 2, 3, 4, and 5; each R⁵ is independently selected from the group consisting of C₁₋₃ alkyl, C₂₋₃ alkenyl, and H; each R⁶ is independently selected from the group consisting of C₁₋₃ alkyl, C₂₋₃ alkenyl, and H; M and M′ are each independently selected from the group consisting of —C(O)O— and —OC(O)—; R^{b} is a C₁₋₁₂ alkyl or C₂₋₁₂ alkenyl; l is selected from the group consisting of 1, 2, 3, 4, and 5; and m is selected from the group consisting of 5, 6, 7, 8, 9, 10, 11, 12, and 13.

In some embodiments of the compounds of Formula (IC), R′ is

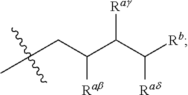

wherein

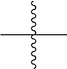

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R^{b} is a C₁₋₁₂ alkyl; l is 5; and m is 7.

In some embodiments of the compounds of Formula (IC), R′ is

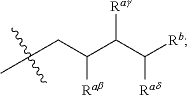

wherein

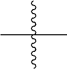

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R^{b} is a C₁₋₁₂ alkyl; l is 3; and m is 7.

In some embodiments of the compounds of Formula (IC), R′ is

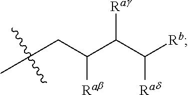

wherein

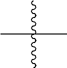

denotes a point of attachment; R^{aβ} and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R^{b} is a C₁₋₁₂ alkyl; l is 5; and m is 7.

In some embodiments of the compounds of Formula (IC), R′ is

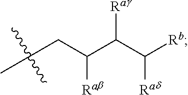

wherein

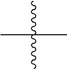

denotes a point of attachment; wherein R^{aα}, R^{aβ}, R^{aγ}, and R^{aδ} are each independently selected from the group consisting of H, C₂₋₁₂ alkyl, and C₂₋₁₂ alkenyl; R² and R³ are each independently selected from the group consisting of C₁₋₁₄ alkyl and C₂₋₁₄ alkenyl; R⁴ is

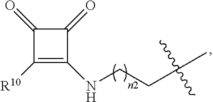

wherein

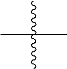

denotes a point of attachment; wherein R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10; each R⁵ is independently selected from the group consisting of C₁₋₃ alkyl, C₂₋₃ alkenyl, and H; each R⁶ is independently selected from the group consisting of C₁₋₃ alkyl, C₂₋₃ alkenyl, and H; M and M′ are each independently selected from the group consisting of —C(O)O— and —OC(O)—; R^{b} is a C₁₋₁₂ alkyl or C₂₋₁₂ alkenyl; l is selected from the group consisting of 1, 2, 3, 4, and 5; and m is selected from the group consisting of 5, 6, 7, 8, 9, 10, 11, 12, and 13.

In some embodiments of the compounds of Formula (IC), R′ is

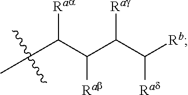

wherein

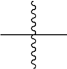

denotes a point of attachment; R^{aβ}, R^{aγ}, and R^{aδ} are each H; R^{aα} is C₂₋₁₂ alkyl; R² and R³ are each C₁₋₁₄ alkyl; R⁴ is

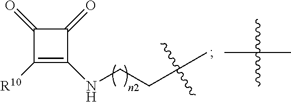

denotes a point of attachment; R¹⁰ is NH(C₁₋₆ alkyl); n2 is 2; each R⁵ is H; each R⁶ is H; M and M′ are each —C(O)O—; R^{b} is a C₁₋₁₂ alkyl; l is 5; and m is 7.

In some embodiments, the compound of Formula (IC) is:

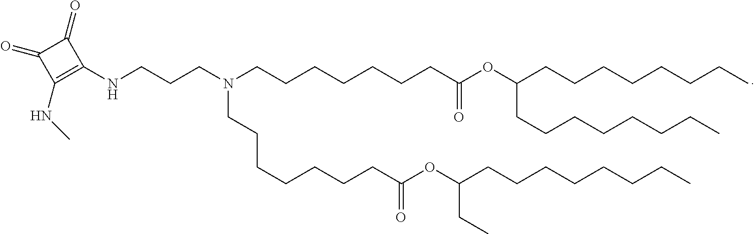 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In certain embodiments, a subset of compounds of Formula (I) includes those of Formula (II):

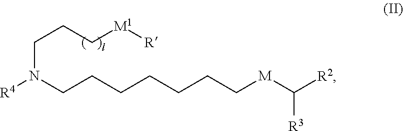

or its N-oxide, or a salt or isomer thereof, wherein 1 is selected from 1, 2, 3, 4, and 5; M¹ is a bond or M′; R⁴ is hydrogen, unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R⁸, —NHC(═NR⁹)N(R)₂, —NHC(═CHR⁹)N(R)₂, —OC(O)N(R)₂, —N(R)C(O)OR, heteroaryl or heterocycloalkyl; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl.

In one embodiment, the compounds of Formula (I) are of Formula (IIa),

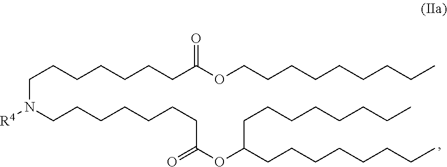

or their N-oxides, or salts or isomers thereof, wherein is as described herein.

In another embodiment, the compounds of Formula (I) are of Formula (IIb),

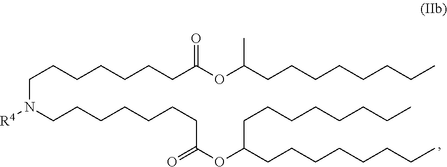

or their N-oxides, or salts or isomers thereof, wherein R⁴ is as described herein.

In another embodiment, the compounds of Formula (I) are of Formula (IIc) or (IIe):

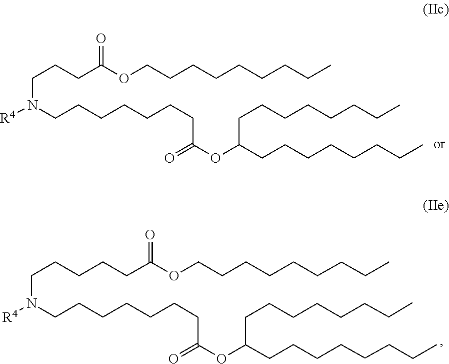

or their N-oxides, or salts or isomers thereof, wherein R⁴ is as described herein.

In another embodiment, the compounds of Formula (I) are of Formula (IIf):

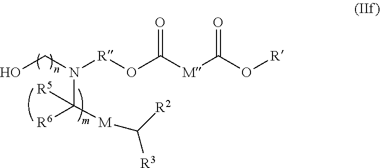

or their N-oxides, or salts or isomers thereof,
wherein M is —C(O)O— or —OC(O)—, M″ is C₁₋₆ alkyl or C₂₋₆ alkenyl, R² and R³ are independently selected from the group consisting of C₅₋₁₄ alkyl and C₅₋₁₄ alkenyl, and n is selected from 2, 3, and 4.

In a further embodiment, the compounds of Formula (I) are of Formula (IId),

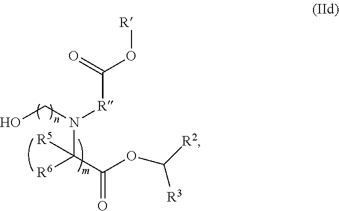

or their N-oxides, or salts or isomers thereof, wherein n is 2, 3, or 4; and m, R′, R″, and R² through R⁶ are as described herein. For example, each of R² and R³ may be independently selected from the group consisting of C₅₋₁₄ alkyl and C₅₋₁₄ alkenyl.

In a further embodiment, the compounds of Formula (I) are of Formula (IIg),

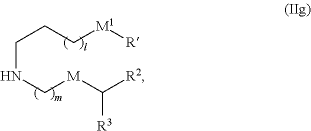

or their N-oxides, or salts or isomers thereof, wherein l is selected from 1, 2, 3, 4, and 5; m is selected from 5, 6, 7, 8, and 9; M¹ is a bond or M′; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R² and R³ are independently selected from the group consisting of C₅₋₁₄ alkyl and C₅₋₁₄ alkenyl.

In a further embodiment, the compounds of Formula (I) are of Formula (IIh):

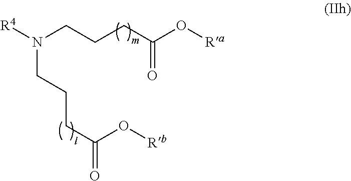

or its N-oxide, or a salt or isomer thereof, wherein R′^{a} is R′^{branched} or R′^{cyclic};

wherein R^{′branched} is:

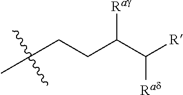

### and R^{′cyclic} is:

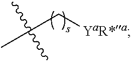

and

R^{′b} is:

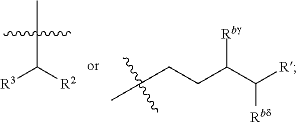

wherein

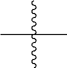

denotes a point of attachment;

R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;

R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;

R² and R³ are each independently selected from the group consisting of C₁₋₁₄ alkyl and C₂₋₁₄ alkenyl;

R⁴ is selected from the group consisting of —(CH₂)ₙOH wherein n is selected from the group consisting of 1, 2, 3, 4, and 5, and

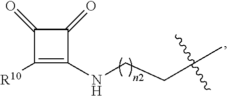

wherein

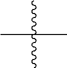

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;

each R′ independently is a C₁₋₁₂ alkyl or C₂₋₁₂ alkenyl;

Y^{a} is a C₃₋₆ carbocycle;

R*^{″a} is selected from the group consisting of C₁₋₁₅ alkyl and C₂₋₁₅ alkenyl; and

s is 2 or 3;

m is selected from 1, 2, 3, 4, 5, 6, 7, 8, and 9;

l is selected from 1, 2, 3, 4, 5, 6, 7, 8, and 9.

In some embodiments, the compounds of Formula (I) are of Formula (IIh):

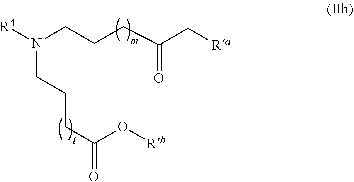

or its N-oxide, or a salt or isomer thereof,

wherein R^{′a} is R^{′branched}; wherein

R^{′branched} is:

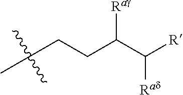

### and R^{′b} is:

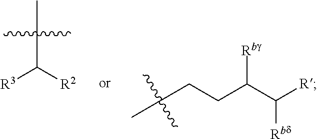

wherein

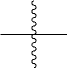

denotes a point of attachment;

R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;

R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;

R² and R³ are each independently selected from the group consisting of C₁₋₁₄ alkyl and C₂₋₁₄ alkenyl;

R⁴ is selected from the group consisting of —(CH₂)ₙOH wherein n is selected from the group consisting of 1, 2, 3, 4, and 5, and

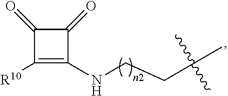

wherein

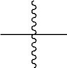

denotes a point of attachment; wherein
R¹⁰ is N(R)₂; each is independently selected from the group consisting of C₁₋₆ alkyl, C₂₋₃ alkenyl, and H; and n2 is selected from the group consisting of 1, 2, 3, 4, 5, 6, 7, 8, 9, and 10;

each R′ independently is a C₁₋₁₂ alkyl or C₂₋₁₂ alkenyl;

m is selected from 1, 2, 3, 4, 5, 6, 7, 8, and 9;

l is selected from 1, 2, 3, 4, 5, 6, 7, 8, and 9.

In some embodiments, the compounds of Formula (I) are of Formula (IIh):

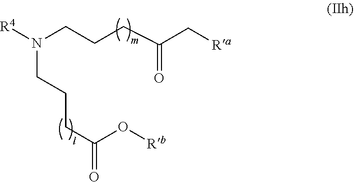

or its N-oxide, or a salt or isomer thereof,

wherein R^{′a} is R^{′branched}; wherein

R^{′branched} is:

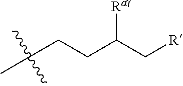

and R^{′b} is:

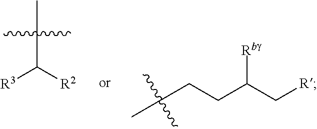

wherein

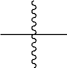

denotes a point of attachment;

R^{aγ} and R^{bγ} are each independently selected from the group consisting of C₁₋₁₂ alkyl and C₂₋₁₂ alkenyl;

R² and R³ are each independently selected from the group consisting of C₁₋₁₄ alkyl and C₂₋₁₄ alkenyl;

R⁴ is selected from the group consisting of —(CH₂)ₙOH wherein n is selected from the group consisting of 1, 2, 3, 4, and 5, and

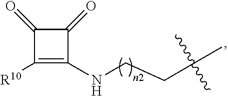

wherein

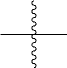

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;

each R′ independently is a C₁₋₁₂ alkyl or C₂₋₁₂ alkenyl;

m is selected from 1, 2, 3, 4, 5, 6, 7, 8, and 9;

l is selected from 1, 2, 3, 4, 5, 6, 7, 8, and 9.

In some embodiments, the compounds of Formula (I) are of Formula (IIh):

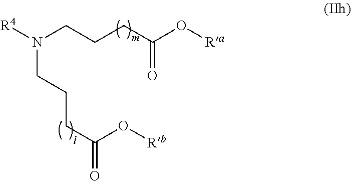

or its N-oxide, or a salt or isomer thereof,

wherein R^{′a} is R^{′branched}; wherein

R^{′branched} is:

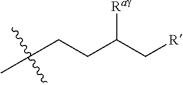

### and R^{′b} is:

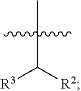

wherein

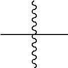

denotes a point of attachment;

wherein R^{aγ} is selected from the group consisting of C₁₋₁₂ alkyl and C₂₋₁₂ alkenyl;

R² and R³ are each independently selected from the group consisting of C₁₋₁₄ alkyl and C₂₋₁₄ alkenyl;

R⁴ is selected from the group consisting of —(CH₂)ₙOH wherein n is selected from the group consisting of 1, 2, 3, 4, and 5, and

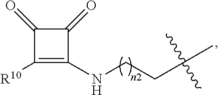

wherein

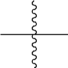

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;

R′ is a C₁₋₁₂ alkyl or C₂₋₁₂ alkenyl;

m is selected from 1, 2, 3, 4, 5, 6, 7, 8, and 9;

l is selected from 1, 2, 3, 4, 5, 6, 7, 8, and 9.

In some embodiments, the compounds of Formula (I) are of Formula (IIh):

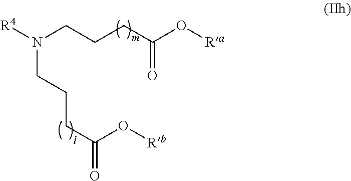

or its N-oxide, or a salt or isomer thereof,

wherein R^{′a} is R^{′branched}; wherein

R^{′branched} is:

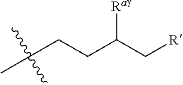

### and R^{′b} is:

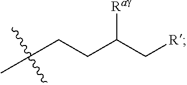

wherein

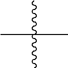

denotes a point of attachment;

wherein R^{aγ} and R^{bγ} are each independently selected from the group consisting of C₁₋₁₂ alkyl and C₂₋₁₂ alkenyl;

R⁴ is selected from the group consisting of —(CH₂)ₙOH wherein n is selected from the group consisting of 1, 2, 3, 4, and 5, and

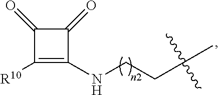

wherein

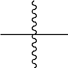

denotes a point of attachment; wherein

R¹⁰ is N(R)₂; each R is independently selected from the group consisting of C₁₋₆ alkyl, C₂-3 alkenyl, and H; and n2 is selected from the group consisting of 1, 2, 3, 4, 5, 6, 7, 8, 9, and 10;

each R′ independently is a C₁₋₁₂ alkyl or C₂₋₁₂ alkenyl;

m is selected from 1, 2, 3, 4, 5, 6, 7, 8, and 9;

l is selected from 1, 2, 3, 4, 5, 6, 7, 8, and 9.

In some embodiments, the compounds of Formula (I) are of Formula (IIh):

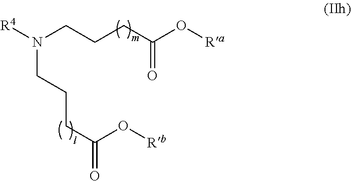

its N-oxide, or a salt or isomer thereof,

wherein R^{′a} is R^{′branched}; wherein

R^{′branched} is:

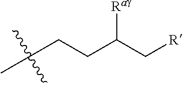

### and R^{′b} is:

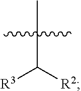

wherein

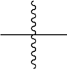

denotes a point of attachment;

wherein R^{aγ} is selected from the group consisting of C₁₋₁₂ alkyl and C₂₋₁₂ alkenyl;

R² and R³ are each independently selected from the group consisting of C₁₋₁₄ alkyl and C₂₋₁₄ alkenyl;

R⁴ is —(CH₂)ₙOH wherein n is selected from the group consisting of 1, 2, 3, 4, and 5;

R′ is a C₁₋₁₂ alkyl or C₂₋₁₂ alkenyl;

m is selected from 1, 2, 3, 4, 5, 6, 7, 8, and 9;

l is selected from 1, 2, 3, 4, 5, 6, 7, 8, and 9.

In some embodiments of the compound of Formula (IIh), m and 1 are each independently selected from 4, 5, and 6. In some embodiments of the compound of Formula (IIh), m and 1 are each 5.

In some embodiments of the compound of Formula (IIh), each R′ independently is a C₁₋₁₂ alkyl. In some embodiments of the compound of Formula (IIh), each R′ independently is a C₂₋₅ alkyl.

In some embodiments of the compound of Formula (IIh), R^{′b} is:

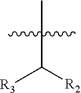

and R² and R³ are each independently a C₁₋₁₄ alkyl. In some embodiments of the compound of Formula (IIh), R^{′b} is:

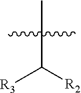

and R² and R³ are each independently a C₆₋₁₀ alkyl. In some embodiments of the compound of Formula (IIh), R^{′b} is:

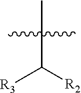

and R² and R³ are each a C₈ alkyl.

In some embodiments of the compound of Formula (IIh), R^{′branched} is:

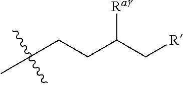

### and R^{′b} is

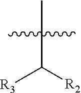

R^{aγ} is a C₁₋₁₂ alkyl, and R² and R³ are each independently a C₆₋₁₀ alkyl. In some embodiments of the compound of Formula (IIh), R^{′branched} is:

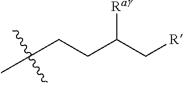

### and R^{′b} is:

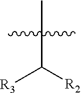

R^{aγ} is a C₂₋₆ alkyl, and R² and R³ are each independently a C₆₋₁₀ alkyl. In some embodiments of the compound of Formula (IIh). R^{′branched} is:

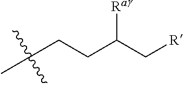

### and R^{′b} is:

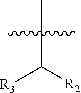

R^{aγ} is a C₂₋₆ alkyl, and R² and R³ are each a C₈ alkyl.

In some embodiments of the compound of Formula (IIh), R^{′branched} is:

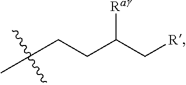

### R^{′b} is:

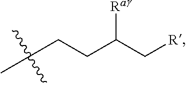

and R^{aγ} and R^{bγ} are each a C₁₋₁₂ alkyl. In some embodiments of the compound of Formula (IIh), R^{′branched} is:

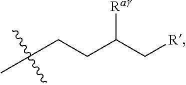

### R^{′b} is:

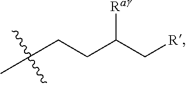

and R^{aγ} and R^{bγ} are each a C₂₋₆ alkyl.

In some embodiments of the compound of Formula (IIh), m and 1 are each independently selected from 4, 5, and 6 and each R′ independently is a C₁₋₁₂ alkyl. In some embodiments of the compound of Formula (IIh), m and 1 are each 5 and each R′ independently is a C₂₋₅ alkyl.

In some embodiments of the compound of Formula (IIh), R^{′branched} is:

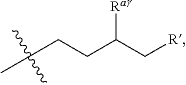

### R^{′b} is:

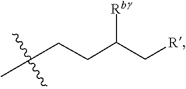

m and l are each independently selected from 4, 5, and 6, each R′ independently is a C₁₋₁₂ alkyl, and R^{aγ} and R^{bγ} are each a C₁₋₁₂ alkyl. In some embodiments of the compound of Formula (IIh), R^{′branched} is:

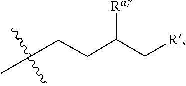

### R′^{b} is:

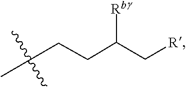

m and l are each 5, each R′independently is a C₂₋₅ alkyl, and R^{aγ} and R^{bγ} are each a C₂₋₆ alkyl.

In some embodiments of the compound of Formula (IIh), R^{′branched} is:

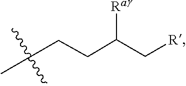

### and R^{′b} is:

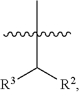

m and l are each independently selected from 4, 5, and 6, R′ is a C₁₋₁₂ alkyl, R^{aγ} is a C₁₋₁₂ alkyl and R² and R³ are each independently a C₆₋₁₀ alkyl. In some embodiments of the compound of Formula (IIh), R^{′branched} is:

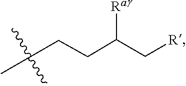

### and R′b is:

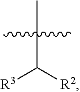

m and l are each 5, R′ is a C₂₋₅ alkyl, R_{aγ} is a C₂₋₆ alkyl, and R² and R³ are each a C₈ alkyl.

In some embodiments of the compound of Formula (IIh), R₄ is

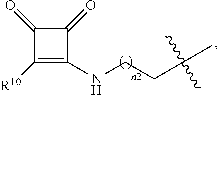

wherein R¹⁰ is NH(C₁₋₆ alkyl) and n2 is 2. In some embodiments of the compound of Formula (IIh), R⁴ is

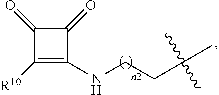

wherein R¹⁰ is NH(CH₃) and n2 is 2.

In some embodiments of the compound of Formula (IIh), R^{′branched} is:

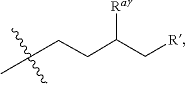

### R^{′b} is:

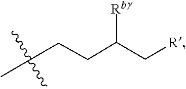

m and l are each independently selected from 4, 5, and 6, each R′ independently is a C₁₋₁₂ alkyl, R^{aγ} and R^{bγ} are each a C₁₋₁₂ alkyl, and R⁴ is

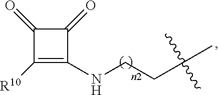

wherein R¹⁰ is NH(C₁₋₆ alkyl), and n2 is 2. In some embodiments of the compound of Formula (IIh), R^{′branched} is:

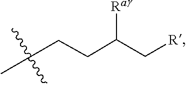

### R^{′b} is:

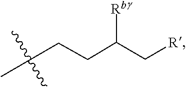

m and l are each 5, each R′ independently is a C₂₋₅ alkyl, R^{aγ} and R^{bγ} are each a C₂₋₆ alkyl, and R⁴ is

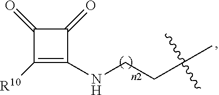

wherein R¹⁰ is NH(CH₃) and n2 is 2.

In some embodiments of the compound of Formula (IIh), R^{′branched} is:

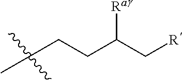

### and R^{′b} is:

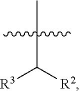

m and l are each independently selected from 4, 5, and 6, R′ is a C₁₋₁₂ alkyl, R² and R³ are each independently a C₆₋₁₀ alkyl, R^{aγ} is a C₁₋₁₂ alkyl, and R⁴ is

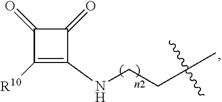

wherein R¹⁰ is NH(C₁₋₆ alkyl) and n2 is 2. In some embodiments of the compound of Formula (IIh), R^{′branched} is:

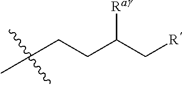

### and R′^{b} is:

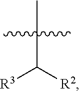

m and l are each 5, R′ is a C₂₋₅ alkyl, R^{aγ} is a C₂₋₆ alkyl, R² and R³ are each a C₈ alkyl, and R⁴ is

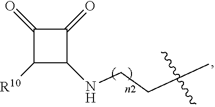

wherein R¹⁰ is NH(CH₃) and n2 is 2.

In some embodiments of the compound of Formula (IIh), R⁴ is —(CH₂)ₙOH and n is 2, 3, or 4. In some embodiments of the compound of Formula (IIh), R⁴ is —(CH₂)ₙOH and n is 2.

In some embodiments of the compound of Formula (IIh), R^{′branched} is:

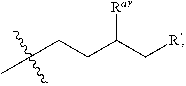

### R^{′b} is:

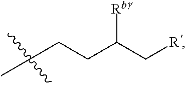

m and l are each independently selected from 4, 5, and 6, each R′ independently is a C₁₋₁₂ alkyl, R^{aγ} and R^{bγ} are each a C₁₋₁₂ alkyl, R⁴ is —(CH₂)ₙOH, and n is 2, 3, or 4. In some embodiments of the compound of Formula (IIh), R^{′branched} is:

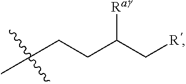

### R^{′b} is:

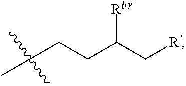

m and l are each 5, each R′ independently is a C₂₋₅ alkyl, R^{aγ} and R^{bγ} are each a C₂₋₆ alkyl, R⁴ is —(CH₂)ₙOH, and n is 2.

In some embodiments, the compounds of Formula (I) are of Formula (IIh):

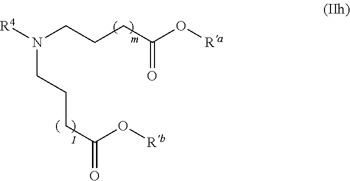

or its N-oxide, or a salt or isomer thereof,

wherein R^{′a} is R^{′branched}; wherein

R^{′branched} is:

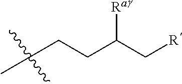

### and R^{′b} is:

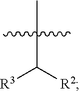

wherein

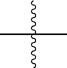

denotes a point of attachment;

R^{aγ} is a C₁₋₁₂ alkyl;

R² and R³ are each independently a C₁₋₁₄ alkyl;

R⁴ is —(CH₂)ₙOH wherein n is selected from the group consisting of 1, 2, 3, 4, and 5;

R′ is a C₁₋₁₂ alkyl;

m is selected from 4, 5, and 6; and

l is selected from 4, 5, and 6.

In some embodiments of the compound of Formula (IIh), m and 1 are each 5, and n is 2, 3, or 4.

In some embodiments of the compound of Formula (IIh) R′ is a C₂₋₅ alkyl, R^{aγ} is a C2-6 alkyl, and R² and R³ are each a C₆₋₁₀ alkyl.

In some embodiments of the compound of Formula (IIh), m and 1 are each 5, n is 2, 3, or 4, R′ is a C₂₋₅ alkyl, R^{aγ} is a C₂₋₆ alkyl, and R² and R³ are each a C₆₋₁₀ alkyl.

In some embodiments, the compounds of Formula (I) are of Formula (IIi):

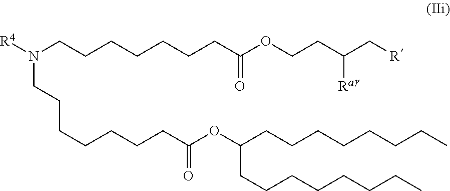

or its N-oxide, or a salt or isomer thereof, wherein

R^{aγ} is a C₂₋₆ alkyl;

R′ is a C₂₋₅ alkyl; and

R⁴ is selected from the group consisting of —(CH₂)ₙOH wherein n is selected from the group consisting of 3, 4, and 5, and

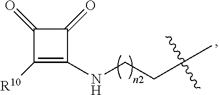

wherein

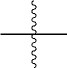

denotes a point of attachment, R¹⁰ is NH(C₁₋₆ alkyl), and n2 is selected from the group consisting of 1, 2, and 3.

In some embodiments, the compounds of Formula (I) are of Formula (IIj):

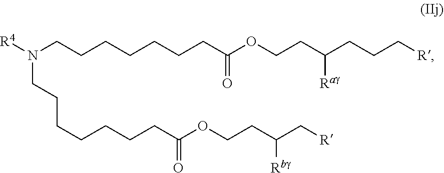

wherein

R^{aγ} and R^{bγ} are each independently a C₂₋₆ alkyl;

each R′ independently is a C₂₋₅ alkyl; and

R⁴ is selected from the group consisting of —(CH₂)ₙOH wherein n is selected from the group consisting of 3, 4, and 5, and

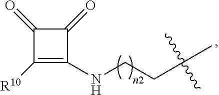

wherein

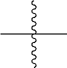

denotes a point of attachment, R¹⁰ is NH(C₁₋₆ alkyl), and n2 is selected from the group consisting of 1, 2, and 3.

In some embodiments of the compound of Formula (IIi) or (IIj), R⁴ is

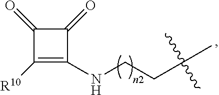

wherein
R¹⁰ is NH(CH₃) and n2 is 2.

In some embodiments of the compound of Formula (IIi) or (IIj), R⁴ is —(CH₂)₂OH.

In some embodiments, the amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

In some embodiments, a compound of Formula (I) is selected from:

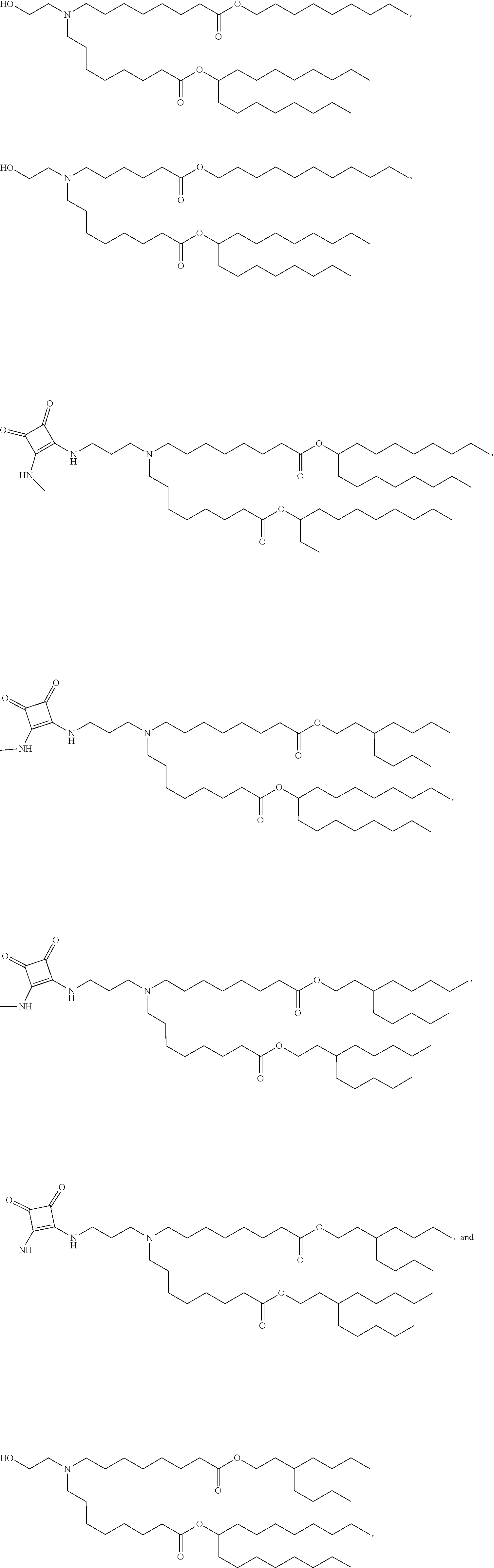

or its N-oxide, or a salt or isomer thereof.

In some embodiments, the compound of Formula (I) is:

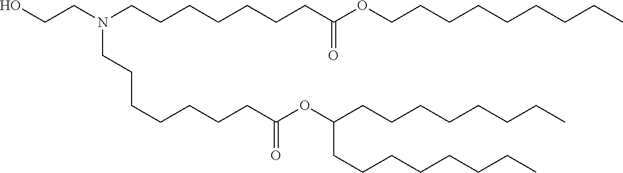 [SMILES: CCCCCCCCCOC(=O)CCCCCCCN(CCO)CCCCCCCC(=O)OC(CCCCCCCC)CCCCCCCC]

or its N-oxide, or a salt or isomer thereof.

In some embodiments, the compound of Formula (I) is:

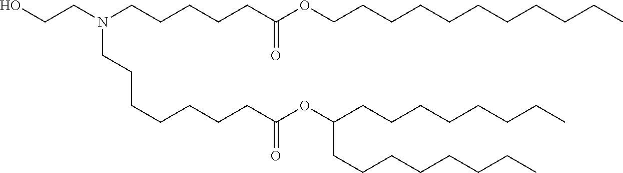 [SMILES: CCCCCCCCCCCOC(=O)CCCCCN(CCO)CCCCCCCC(=O)OC(CCCCCCCC)CCCCCCCC]

or its N-oxide, or a salt or isomer thereof.

In some embodiments, the compound of Formula (I) is:

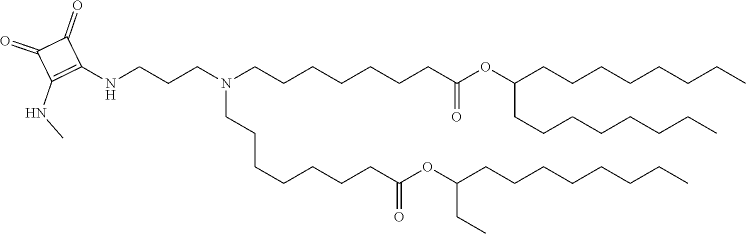 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

or its N-oxide, or a salt or isomer thereof.

In some embodiments, the compound of Formula (I) is:

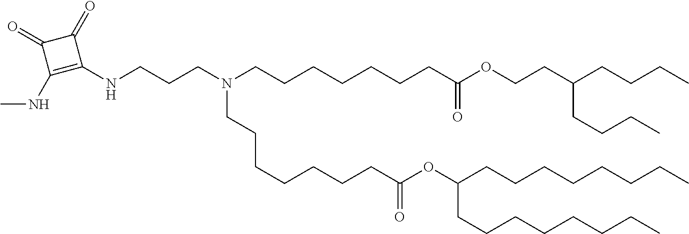 [SMILES: CCCCCCCCC(CCCCCCCC)OC(=O)CCCCCCCN(CCCCCCCC(=O)OCCC(CCCC)CCCC)CCCNc1c(NC)c(=O)c1=O]

or its N-oxide, or a salt or isomer thereof.

In some embodiments, the compound of Formula (I) is:

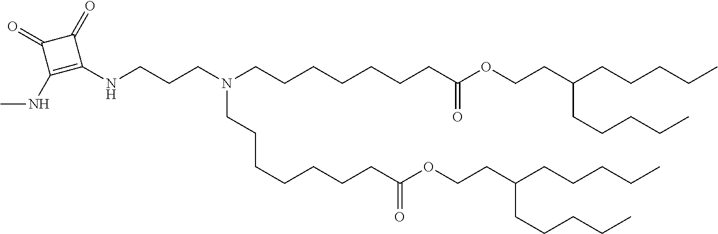 [SMILES: CCCCCC(CCCCC)CCOC(=O)CCCCCCCN(CCCCCCCC(=O)OCCC(CCCCC)CCCCC)CCCNc1c(NC)c(=O)c1=O]

or its N-oxide, or a salt or isomer thereof.

In some embodiments, the compound of Formula (I) is:

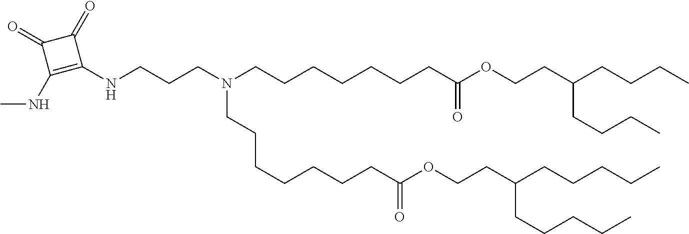 [SMILES: CCCCCC(CCCCC)CCOC(=O)CCCCCCCN(CCCCCCCC(=O)OCCC(CCCC)CCCC)CCCNc1c(NC)c(=O)c1=O]

or its N-oxide, or a salt or isomer thereof.

In some embodiments, the compound of Formula (I) is:

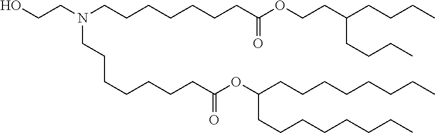 [SMILES: CCCCCCCCC(CCCCCCCC)OC(=O)CCCCCCCN(CCO)CCCCCCCC(=O)OCCC(CCCC)CCCC]

or its N-oxide, or a salt or isomer thereof.

In some embodiments, the amino lipid is

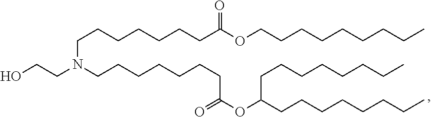 [SMILES: CCCCCCCCCOC(=O)CCCCCCCN(CCO)CCCCCCCC(=O)OC(CCCCCCCC)CCCCCCCC]

or a salt thereof.

In some embodiments, the amino lipid is

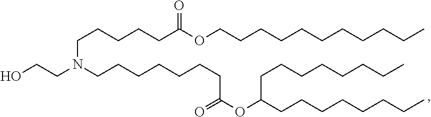 [SMILES: CCCCCCCCCCCOC(=O)CCCCCN(CCO)CCCCCCCC(=O)OC(CCCCCCCC)CCCCCCCC]

or a salt thereof.

The central amine moiety of a lipid according to Formula (I), (IA), (IB), (IC), (II), (IIa), (IIb), (IIc), (IId), (IIe), (IIf), or (IIg) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

In some aspects, the amino lipids of the present disclosure may be one or more of compounds of formula (III),

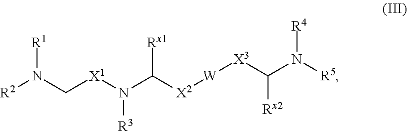

or salts or isomers thereof, wherein

W is

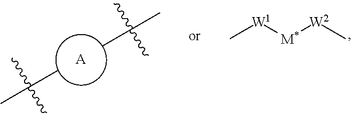

ring A is

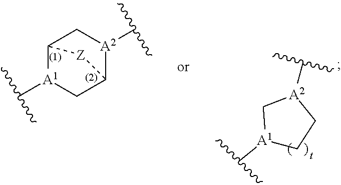

t is 1 or 2;

A¹ and A² are each independently selected from CH or N;

Z is CH₂ or absent wherein when Z is CH₂, the dashed lines (1) and (2) each represent a single bond; and when Z is absent, the dashed lines (1) and (2) are both absent;

R¹, R², R³, R⁴, and R⁵ are independently selected from the group consisting of C₅₋₂₀ alkyl, C₅₋₂₀ alkenyl, —R″MR′, —R*YR″, —YR″, and —R*OR″;

R^{X1} and R^{X2} are each independently H or C₁₋₃ alkyl;

each M is independently selected from the group consisting

of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;

M* is C₁-C₆ alkyl,

W¹ and W² are each independently selected from the group consisting of —O— and —N(R⁶)—;

each R⁶ is independently selected from the group consisting of H and C₁₋₅ alkyl;

X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;

each Y is independently a C₃₋₆ carbocycle;

each R* is independently selected from the group consisting of C₁₋₁₂ alkyl and C₂₋₁₂ alkenyl;

each R is independently selected from the group consisting of C₁₋₃ alkyl and a C₃₋₆ carbocycle;

each R′ is independently selected from the group consisting of C₁₋₁₂ alkyl, C₂₋₁₂ alkenyl, and H;

each R″ is independently selected from the group consisting of C₃₋₁₂ alkyl, C₃₋₁₂ alkenyl and —R*MR′; and

n is an integer from 1-6;

wherein when ring A is

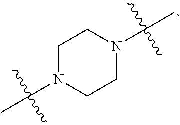 [SMILES: *N1CCN(*)CC1]

then

i) at least one of X¹, X², and X³ is not —CH₂—; and/or

ii) at least one of R¹, R², R³, R⁴, and R⁵ is —R″MR′.

In some embodiments, the compound is of any of formulae (IIIa1)-(IIIa8):

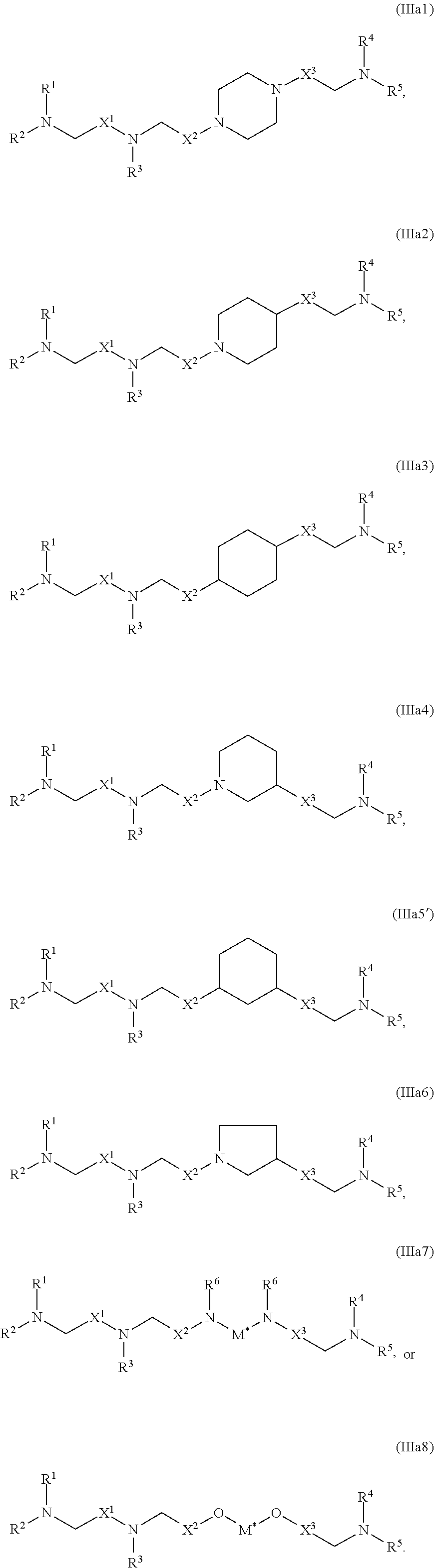

In some embodiments, the amino lipid is

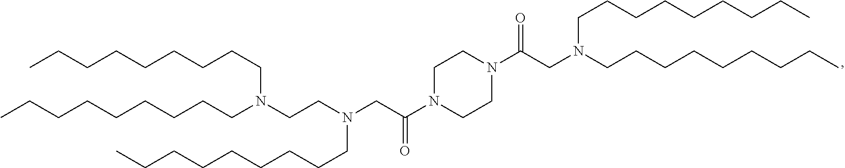 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (III), (IIIa1), (IIIa2), (IIIa3), (IIIa4), (IIIa5), (IIIa6), (IIIa7), or (IIIa8) may be protonated at a physiological pH. Thus, a lipid may have a positive or partial positive charge at physiological pH.

### Phospholipids

The lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In certain embodiments, a phospholipid useful or potentially useful in the present invention is an analog or variant of DSPC. In certain embodiments, a phospholipid useful or potentially useful in the present invention is a compound of Formula (IV):

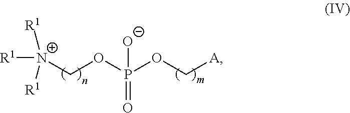

or a salt thereof, wherein:

each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;

n is 1, 2, 3, 4, 5, 6, 7, 8, 9, or 10;

m is 0, 1, 2, 3, 4, 5, 6, 7, 8, 9, or 10;

A is of the formula:

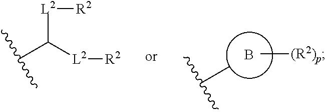

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}) C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}) NR^{N}C(═NR^{N})N(R^{N}), C(S), C(S)N(R^{N}), NR^{N}C(S), NR^{N}C(S)N(R^{N}) S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl,

optionally substituted aryl, or optionally substituted heteroaryl; and

p is 1 or 2;

provided that the compound is not of the formula:

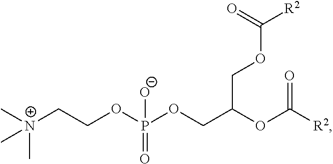

wherein each instance of R² is independently unsubstituted alkyl, unsubstituted alkenyl, or unsubstituted alkynyl.

In some embodiments, the phospholipids may be one or more of the phospholipids described in U.S. Application No. 62/520,530, or in International Application PCT/US2018/037922 filed on 15 Jun. 2018, the entire contents of each of which is hereby incorporated by reference in its entirety.

### Structural Lipids

The lipid composition of a pharmaceutical composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

### Polyethylene Glycol (PEG)-Lipids

The lipid composition of a pharmaceutical composition disclosed herein can comprise one or more polyethylene glycol (PEG) lipids.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

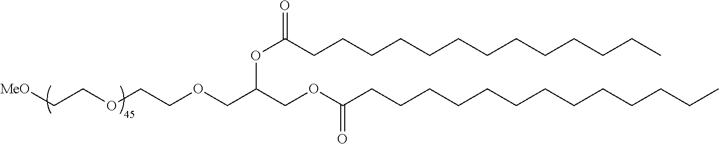

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (V). Provided herein are compounds of Formula (V):

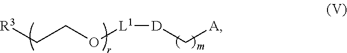

or salts thereof, wherein:

R³ is —OR^{O};

R^{O} is hydrogen, optionally substituted alkyl, or an oxygen protecting group;

r is an integer between 1 and 100, inclusive;

L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O) C(O)O, OC(O), OC(O)O, OC(O)N(R^{N}), NR^{N}C(O)O, or NR^{N}C(O)N(R^{N});

D is a moiety obtained by click chemistry or a moiety cleavable under physiological conditions;

m is 0, 1, 2, 3, 4, 5, 6, 7, 8, 9, or 10;

A is of the formula:

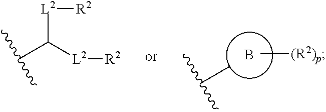

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}) C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}) NR^{N}C(═NR^{N})N(R^{N}), C(S), C(S)N(R^{N}), NR^{N}C(S), NR^{N}C(S)N(R^{N}) S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2.

In certain embodiments, the compound of Formula (V) is a PEG-OH lipid (i.e., R³ is —OR^{O}, and R^{O} is hydrogen). In certain embodiments, the compound of Formula (V) is of Formula (V-OH):

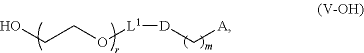

or a salt thereof.

In certain embodiments, a PEG lipid useful in the present invention is a PEGylated fatty acid. In certain embodiments, a PEG lipid useful in the present invention is a compound of Formula (VI). Provided herein are compounds of Formula (VI-A):

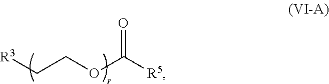

or a salt thereof, wherein:

R³ is-OR^{O};

R^{O} is hydrogen, optionally substituted alkyl or an oxygen protecting group;

r is an integer between 1 and 100, inclusive;

R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O C(O)S, SC(O), C(═NR^{N}), C(═NR^{N})N(R^{N}), NR^{N}C(═NR^{N}) NR^{N}C(═NR^{N})N(R^{N}), C(S), C(S)N(R^{N}), NR^{N}C(S), —NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O; and

each instance of R^{N} is independently hydrogen, optionally substituted alkyl, or a nitrogen protecting group.

In certain embodiments, the compound of Formula (VI) is of Formula (VI-OH):

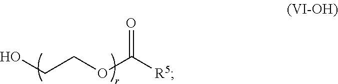

also referred to as (VI-B), or a salt thereof. In some embodiments, r is 40-50.

In yet other embodiments the compound of Formula (VI-C) is:

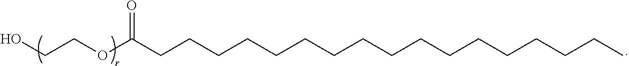

or a salt thereof.

In one embodiment, the compound of Formula (VI-D) is

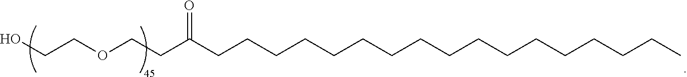

In some aspects, the lipid composition of the pharmaceutical compositions disclosed herein does not comprise a PEG-lipid.

In some embodiments, the PEG-lipids may be one or more of the PEG lipids described in U.S. Application No. U.S. Ser. No. 15/674,872.

In some embodiments, an LNP of the invention comprises an amino lipid of any of Formula I, II or III, a phospholipid comprising DSPC, a structural lipid, and a PEG lipid comprising PEG-DMG.

In some embodiments, an LNP of the invention comprises an amino lipid of any of Formula I, II or III, a phospholipid comprising DSPC, a structural lipid, and a PEG lipid comprising a compound having Formula VI.

In some embodiments, an LNP of the invention comprises an amino lipid of Formula I, II or III, a phospholipid comprising a compound having Formula IV, a structural lipid, and the PEG lipid comprising a compound having Formula V or VI.

In some embodiments, an LNP of the invention comprises an amino lipid of Formula I, II or III, a phospholipid comprising a compound having Formula IV, a structural lipid, and the PEG lipid comprising a compound having Formula V or VI.

In some embodiments, an LNP of the invention comprises an amino lipid of Formula I, II or III, a phospholipid having Formula IV, a structural lipid, and a PEG lipid comprising a compound having Formula VI.

In some embodiments, an LNP of the invention comprises an N:P ratio of from about 2:1 to about 30:1.

In some embodiments, an LNP of the invention comprises an N:P ratio of about 6:1.

In some embodiments, an LNP of the invention comprises an N:P ratio of about 3:1, 4:1, or 5:1.

In some embodiments, an LNP of the invention comprises a wt/wt ratio of the amino lipid component to the RNA of from about 10:1 to about 100:1.

In some embodiments, an LNP of the invention comprises a wt/wt ratio of the amino lipid component to the RNA of about 20:1.

In some embodiments, an LNP of the invention comprises a wt/wt ratio of the amino lipid component to the RNA of about 10:1.

In some embodiments, an LNP of the invention has a mean diameter from about 30 nm to about 150 nm.

In some embodiments, an LNP of the invention has a mean diameter from about 60 nm to about 120 nm.

### Exemplary Additional LNP Components

### Surfactants

In certain embodiments, the lipid nanoparticles of the disclosure optionally includes one or more surfactants.

In certain embodiments, the surfactant is an amphiphilic polymer. As used herein, an amphiphilic “polymer” is an amphiphilic compound that comprises an oligomer or a polymer.

For example, an amphiphilic polymer can comprise an oligomer fragment, such as two or more PEG monomer units. For example, an amphiphilic polymer described herein can be PS 20.

For example, the amphiphilic polymer is a block copolymer.

For example, the amphiphilic polymer is a lyoprotectant.

For example, amphiphilic polymer has a critical micelle concentration (CMC) of less than 2×10−4 M in water at about 30° C. and atmospheric pressure.

For example, amphiphilic polymer has a critical micelle concentration (CMC) ranging between about 0.1×10⁻⁴ M and about 1.3×10⁻⁴ M in water at about 30° C. and atmospheric pressure.

For example, the concentration of the amphiphilic polymer ranges between about its CMC and about 30 times of CMC (e.g., up to about 25 times, about 20 times, about 15 times, about 10 times, about 5 times, or about 3 times of its CMC) in the formulation, e.g., prior to freezing or lyophilization.

For example, the amphiphilic polymer is selected from poloxamers (Pluronic®), poloxamines (Tetronic®), polyoxyethylene glycol sorbitan alkyl esters (polysorbates) and polyvinyl pyrrolidones (PVPs).

For example, the amphiphilic polymer is a poloxamer. For example, the amphiphilic polymer is of the following structure:

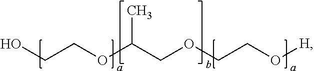

wherein a is an integer between 10 and 150 and b is an integer between 20 and 60. For example, a is about 12 and b is about 20, or a is about 80 and b is about 27, or a is about 64 and b is about 37, or a is about 141 and b is about 44, or a is about 101 and b is about 56.

For example, the amphiphilic polymer is P124, P188, P237, P338, or P407.

For example, the amphiphilic polymer is P188 (e.g., Poloxamer 188, CAS Number 9003-11-6, also known as Kolliphor P188).

For example, the amphiphilic polymer is a poloxamine, e.g., tetronic 304 or tetronic 904.

For example, the amphiphilic polymer is a polyvinylpyrrolidone (PVP), such as PVP with molecular weight of 3 kDa, 10 kDa, or 29 kDa.

For example, the amphiphilic polymer is a polysorbate, such as PS 20.

In certain embodiments, the surfactant is a non-ionic surfactant.

In some embodiments, the lipid nanoparticle comprises a surfactant. In some embodiments, the surfactant is an amphiphilic polymer. In some embodiments, the surfactant is a non-ionic surfactant.

For example, the non-ionic surfactant is selected from the group consisting of polyethylene glycol ether (Brij), poloxamer, polysorbate, sorbitan, and derivatives thereof. For example, the polyethylene glycol ether is a compound of Formula (VIII):

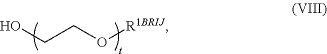

or a salt or isomer thereof, wherein:

t is an integer between 1 and 100;

R^{1BRIJ} independently is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NR^{N}C(O)N(R^{N}) —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and

each instance of R^{N} is independently hydrogen, C₁₋₆ alkyl, or a nitrogen protecting group

In some embodiment, R^{1BRIJ} is C₁₈ alkyl. For example, the polyethylene glycol ether is a compound of Formula (VIII-a):

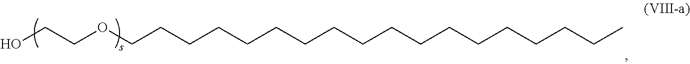

or a salt or isomer thereof.

In some embodiments, R^{1BRIJ} is C₁₈ alkenyl. For example, the polyethylene glycol ether is a compound of Formula (VIII-b):

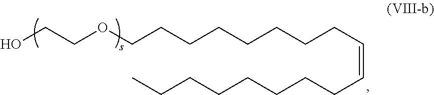

or a salt or isomer thereof.

In some embodiments, the poloxamer is selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407.

In some embodiments, the polysorbate is Tween® 20, Tween® 40, Tween®, 60, or Tween® 80.

In some embodiments, the derivative of sorbitan is Span® 20, Span® 60, Span® 65, Span® 80, or Span® 85.

In some embodiments, the concentration of the non-ionic surfactant in the lipid nanoparticle ranges from about 0.00001% w/v to about 1% w/v, e.g., from about 0.00005% w/v to about 0.5% w/v, or from about 0.0001% w/v to about 0.1% w/v.

In some embodiments, the concentration of the non-ionic surfactant in lipid nanoparticle ranges from about 0.000001 wt % to about 1 wt %, e.g., from about 0.000002 wt % to about 0.8 wt %, or from about 0.000005 wt % to about 0.5 wt %.

In some embodiments, the concentration of the PEG lipid in the lipid nanoparticle ranges from about 0.01% by molar to about 50% by molar, e.g., from about 0.05% by molar to about 20% by molar, from about 0.07% by molar to about 10% by molar, from about 0.1% by molar to about 8% by molar, from about 0.2% by molar to about 5% by molar, or from about 0.25% by molar to about 3% by molar.

### Adjuvants

In some embodiments, an LNP of the invention optionally includes one or more adjuvants, e.g., Glucopyranosyl Lipid Adjuvant (GLA), CpG oligodeoxynucleotides (e.g., Class A or B), poly(I.C), aluminum hydroxide, and Pam3CSK4.

### Other Components

An LNP of the invention may optionally include one or more components in addition to those described in the preceding sections. For example, a lipid nanoparticle may include one or more small hydrophobic molecules such as a vitamin (e.g., vitamin A or vitamin E) or a sterol. Lipid nanoparticles may also include one or more permeability enhancer molecules, carbohydrates, polymers, surface altering agents, or other components. A permeability enhancer molecule may be a molecule described by U.S. patent application publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a lipid nanoparticle. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of an LNP (e.g., by coating, adsorption, covalent linkage, or other process).

A lipid nanoparticle may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of an LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21st Edition, A. R. Gennaro; Lippincott, Williams & Wilkins, Baltimore, Md., 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC® F 68, POLOXAMER® 188, cetrimonium bromide, cetylpyridinium chloride, benzalkonium chloride, docusate sodium, and/or combinations thereof.

A binding agent may be starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN® II, NEOLONE™, KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

Additional and exemplary lipid nanoparticles and compounds are disclosed in International Application No. PCT/US2020/051609, filed Sep. 18, 2020, the entire contents of which are hereby incorporated by reference.

### Methods of Using the LNP Compositions

The present disclosure provides LNP compositions, which can be delivered to cells, e.g., target cells, e.g., in vitro or in vivo. For in vitro protein expression, the cell is contacted with the LNP by incubating the LNP and the cell ex vivo. Such cells may subsequently be introduced in vivo. For in vivo protein expression, the cell is contacted with the LNP by administering the LNP to a subject to thereby increase or induce protein expression in or on the cells within the subject. For example, in one embodiment, the LNP is administered intravenously. In another embodiment, the LNP is administered intramuscularly. In yet other embodiment, the LNP is administered by a route selected from the group consisting of subcutaneously, intranodally and intratumorally.

For in vitro delivery, in one embodiment the cell is contacted with the LNP by incubating the LNP and the target cell ex vivo. In one embodiment, the cell is a human cell. Various types of cells have been demonstrated to be transfectable by the LNP.

In another embodiment, the cell is contacted with the LNP for, e.g., at least 30 minutes, at least 1 hour, at least 2 hours, at least 3 hours, at least 4 hours, at least 5 hours, at least 6 hours, at least 12 hours or at least 24 hours.

In one embodiment, the cell is contacted with the LNP for a single treatment/transfection. In another embodiment, the cell is contacted with the LNP for multiple treatments/transfections (e.g., two, three, four or more treatments/transfections of the same cells).

In another embodiment, for in vivo delivery, the cell is contacted with the LNP by administering the LNP to a subject to thereby deliver the nucleic acid to cells within the subject. For example, in one embodiment, the LNP is administered intravenously. In another embodiment, the LNP is administered intramuscularly. In yet other embodiment, the LNP is administered by a route selected from the group consisting of subcutaneously, intranodally and intratumorally.

In an aspect, provided herein is a method of increasing expression of a therapeutic payload or prophylactic payload in a cell, comprising administering to the cell an LNP composition disclosed herein.

In a related aspect, provided herein is an LNP composition for use in a method of increasing expression of a therapeutic payload or prophylactic payload in a cell.

In another aspect, the disclosure provides a method of increasing expression of a therapeutic payload or prophylactic payload, in a subject, comprising administering to the subject an effective amount of an LNP composition disclosed herein.

In a related aspect, provided herein is an LNP composition for use in a method of increasing expression of a therapeutic payload or prophylactic payload in a subject.

In yet another aspect, provided herein is a method of delivering an LNP composition disclosed herein.

In a related aspect, provided herein is an LNP composition for use in a method of delivering the LNP composition to a cell.

In an embodiment, the method or use, comprises contacting the cell in vitro, in vivo or ex vivo with the LNP composition.

In an embodiment, the LNP compositions of the present disclosure are contacted with cells, e.g., ex vivo or in vivo and can be used to deliver a secreted polypeptide, an intracellular polypeptide or a transmembrane polypeptide to a subject.

In an aspect, the disclosure provides a method of delivering an LNP composition disclosed herein to a subject having a disease or disorder, e.g., as described herein.

In a related aspect, provided herein is an LNP composition for use in a method of delivering the LNP composition to a subject having a disease or disorder, e.g., as described herein.

In another aspect, provided herein is a method of modulating an immune response in a subject, comprising administering to the subject in need thereof an effective amount of an LNP composition disclosed herein.

In a related aspect, provided herein is an LNP composition for use in a method of modulating an immune response in a subject, comprising administering to the subject an effective amount of the LNP composition.

In another aspect, provided herein is a method of delivering a secreted polypeptide, an intracellular polypeptide or a transmembrane polypeptide to a subject.

In an aspect, provided herein is a method of treating, preventing, or preventing a symptom of, a disease or disorder comprising administering to a subject in need thereof an effective amount of an LNP composition disclosed herein.

In a related aspect, provided herein is an LNP composition for use in a method of treating, preventing, or preventing a symptom of, a disease or disorder in a subject, comprising administering to the subject in need thereof an effective amount of the LNP composition.

In some embodiments, the methods or composition for use result in an increased expression and/or level of mRNA encoding the therapeutic payload or prophylactic payload.

In some embodiments, the methods or composition for use result in sustained expression and/or level of mRNA encoding the therapeutic payload or prophylactic payload.

In some embodiments, the methods or composition for use result in increased expression and/or level of therapeutic payload or prophylactic payload.

In some embodiments, the methods or composition for use result in sustained expression and/or level of therapeutic payload or prophylactic payload.

In some embodiments, any one of the functional effects described herein is compared to a cell which:
(a) has not been contacted with the LNP composition disclosed herein; or
(b) has not been contacted with an LNP comprising a polynucleotide comprising a 5′ UTR described herein, a 3′ UTR described herein and/or a coding region comprising a stop element described herein.

### Combination Therapies

In some embodiments, the methods of treatment or compositions for use disclosed herein, comprise administering an LNP disclosed herein in combination with an additional agent. In an embodiment, the additional agent is a standard of care for the disease or disorder, e.g., autoimmune disease. In an embodiment, the additional agent is an mRNA.

In some aspects, the subject for the present methods or compositions has been treated with one or more standard of care therapies. In other aspects, the subject for the present methods or compositions has not been responsive to one or more standard of care therapies or anti-cancer therapies.

### Pharmaceutical Compositions

The present disclosure provides pharmaceutical formulations comprising any of the LNP compositions disclosed herein.

In some embodiments of the disclosure, the polynucleotide is formulated in compositions and complexes in combination with one or more pharmaceutically acceptable excipients. Pharmaceutical compositions can optionally comprise one or more additional active substances, e.g. therapeutically and/or prophylactically active substances. Pharmaceutical compositions of the present disclosure can be sterile and/or pyrogen-free. General considerations in the formulation and/or manufacture of pharmaceutical agents can be found, for example, in Remington: The Science and Practice of Pharmacy 21st ed., Lippincott Williams & Wilkins, 2005.

In some embodiments, compositions are administered to humans, human patients or subjects. For the purposes of the present disclosure, the phrase “active ingredient” generally refers to polynucleotides to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals.

In some embodiments, the polynucleotide of the present disclosure is formulated for subcutaneous, intravenous, intraperitoneal, intramuscular, intra-articular, intra-synovial, intrasternal, intrathecal, intrahepatic, intralesional, intracranial, intraventricular, oral, inhalation spray, pulmonary, topical, rectal, nasal, buccal, vaginal, or implanted reservoir intramuscular, subcutaneous, or intradermal delivery. In other embodiments, the polynucleotide is formulated for subcutaneous or intravenous delivery.

Formulations of the pharmaceutical compositions described herein can be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition can comprise between 0.1% and 100%, e.g., between 0.5% and 50%, between 1% and 30%, between 5% and 80%, or at least 80% (w/w) active ingredient.

### Formulations and Delivery

The polynucleotide comprising an mRNA of the disclosure can be formulated using one or more excipients.

The function of the one or more excipients is, e.g., to: (1) increase stability; (2) increase cell transfection; (3) permit the sustained or delayed release (e.g., from a depot formulation of the polynucleotide); (4) alter the biodistribution (e.g., target the polynucleotide to specific tissues or cell types); (5) increase the translation of encoded protein in vivo; and/or (6) alter the release profile of encoded protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, lipidoids, liposomes, lipid nanoparticles, polymers, lipoplexes, core-shell nanoparticles, peptides, proteins, cells transfected with polynucleotides (e.g., for transplantation into a subject), hyaluronidase, nanoparticle mimics and combinations thereof. Accordingly, the formulations of the disclosure can include one or more excipients, each in an amount that together increases the stability of the polynucleotide, increases cell transfection by the polynucleotide, increases the expression of polynucleotides encoded protein, and/or alters the release profile of polynucleotide encoded proteins. Further, the polynucleotides of the present disclosure can be formulated using self-assembled nucleic acid nanoparticles.

Formulations of the pharmaceutical compositions described herein can be prepared by any method known or hereafter developed in the art of pharmacology. In general, such preparatory methods include the step of associating the active ingredient with an excipient and/or one or more other accessory ingredients.

A pharmaceutical composition in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered. For example, the composition can comprise between 0.1% and 99% (w/w) of the active ingredient. By way of example, the composition can comprise between 0.1% and 100%, e.g., between 0.5 and 50%, between 1-30%, between 5-80%, at least 80% (w/w) active ingredient.

In some embodiments, the formulations described herein contain at least one polynucleotide. As a non-limiting example, the formulations contain 1, 2, 3, 4 or 5 polynucleotides.

Pharmaceutical formulations can additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006). The use of a conventional excipient medium can be contemplated within the scope of the present disclosure, except insofar as any conventional excipient medium can be incompatible with a substance or its derivatives, such as by producing any undesirable biological effect or otherwise interacting in a deleterious manner with any other component(s) of the pharmaceutical composition.

In some embodiments, the particle size of the lipid nanoparticle is increased and/or decreased. The change in particle size can be able to help counter biological reaction such as, but not limited to, inflammation or can increase the biological effect of the modified mRNA delivered to mammals.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients can optionally be included in the pharmaceutical formulations of the disclosure.

In some embodiments, the polynucleotide is administered in or with, formulated in or delivered with nanostructures that can sequester molecules such as cholesterol. Non-limiting examples of these nanostructures and methods of making these nanostructures are described in US Patent Publication No. US20130195759. Exemplary structures of these nanostructures are shown in US Patent Publication No. US20130195759, and can include a core and a shell surrounding the core.

A polynucleotide comprising an mRNA of the disclosure can be delivered to a cell using any method known in the art. For example, the polynucleotide comprising an mRNA of the disclosure can be delivered to a cell by a lipid-based delivery, e.g., transfection, or by electroporation.

### EQUIVALENTS AND SCOPE

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

Table of contents for Examples
5′ UTR

| | |
|---|---|
| Example 1 | Identification of the A1 5′ UTR |
| Example 2 | In vivo increase in protein expression with LNP formulated mRNA having |
| | the A1 5′ UTR |
| Example 3 | Increased target protein expression in primary hepatocytes with LNP |
| | formulated mRNA having the A1 5′ UTR |
| Example 4 | Increased target protein expression in vivo in mouse immune cells with |
| | LNP formulated mRNA having the A1 5′ UTR |
| Example 5 | Increased target protein expression in human PBMCs in vitro with LNP |
| | formulated mRNA having the A1 5′ UTR |
| Example, 6 | In vitro effect of mRNA having the A1 5′ UTR in a rare disease model |
| Example 7 | Protein expression from A1 variants |
| Example 8 | In vivo protein expression from A1 variants |

3′ UTR

| | |
|---|---|
| Example 9 | Discovery and application of 3′UTR sequences for extension of mRNA |
| | half-life |

Stop element

| | |
|---|---|
| Example 10 | Engineering of stop elements |
| Example 11 | Increased expression of a target protein encoded by mRNA having |
| | different stop elements |
| Example 12 | In vivo expression of a target protein encoded by mRNA having different |
| | stop elements |
| Example 13 | Expression of a reporter protein encoded by mRNAs having different stop |
| | elements |

Stabilized tails

| | |
|---|---|
| Example 14 | Stable Tail and Protein Variant Expression |

Use of LNP formulations having one or more mRNA elements described herein

| | |
|---|---|
| Example 15 | In vivo effect of LNP formulated mRNA encoding an immune checkpoint |
| | protein having mRNA elements disclosed herein |
| Example 16 | In vivo expression of an immune checkpoint protein encoded by mRNA |
| | having various mRNA elements in dendritic cells |
| Example 17 | in vivo effect of mRNA having the A1 5′ UTR and/or the B1 3′ UTR |
| Example 18 | Effect of LNP formulated mRNA having various mRNA elements in a |
| | pulmonary disease model |

Stop element

| | |
|---|---|
| Example 19 | Effect of stop codon readthrough in mRNA having different stop elements |
| Example 20 | Expression of a target protein encoded by mRNA having different stop |
| | elements |
| Example 21 | Expression of a target protein encoded by mRNA having different stop |
| | elements |
| Example 22 | In vivo expression of a target protein encoded by mRNA having different |
| | stop elements |
| Example 23 | In vivo expression of a target protein encoded by mRNA having different |
| | stop elements |
| Example 24 | Expression of a target protein encoded by mRNA having different stop |
| | elements |
| Example 25 | Expression of a target protein encoded by mRNA having different stop |
| | elements |
| Example 26 | Expression of a target protein encoded by mRNA having different stop |
| | elements |

One or more mRNA elements

| | |
|---|---|
| Example 27 | In vivo effect of LNP formulated mRNA encoding an immune checkpoint |
| | protein having different mRNA elements |
| Example 28 | In vivo effect of LNP formulated mRNA encoding an immune checkpoint |
| | protein having different mRNA elements |

Stop element

| | |
|---|---|
| Example 29 | Expression of an immune checkpoint protein encoded by mRNA having |
| | different stop elements |
| Example 30 | Expression of an immune checkpoint protein encoded by mRNA having |
| | different stop elements |

One or more mRNA elements

| | |
|---|---|
| Example 31 | In vivo effect of LNP formulated mRNA encoding a target protein having |
| | different mRNA elements |

3′ UTR

| | |
|---|---|
| Example 32 | Expression of a target protein encoded by mRNA having different 3′ UTRs |
| Example 33 | In vivo expression of a target protein encoded by mRNA having different |
| | 3′ UTRs |

5′ UTR

| | |
|---|---|
| Example 34 | Expression of a target protein encoded by mRNA having different 5′ UTRs |
| Example 35 | In vivo expression of a target protein encoded by mRNA having different |
| | 5′ UTRs |

Method of making LNP compositions

| | |
|---|---|
| Example 36 | Production of LNP composition |
| Example 37 | Synthesis of Exemplary Compound of Formula (1) |

### Example 1: Identification of the A1 5′ UTR

This Example describes the identification of the A1 5′ UTR as a candidate from a 5′ UTR screen. For the 5′ UTR screen, sequences from a broad array of sources and combinations thereof were included. Two parameters were measured for each 5′ UTR candidate: (1) expression by eGFP-degron IncuCyte measurements and (2) initiation fidelity by Wes leaky scanning assay. As a comparison, the 5′ UTR performance in both assays was compared to the A11 reference 5′ UTR. A cap comprising the sequence of GG was used in this Example.

From the screen, several 5′ UTR sequences were associated with increased expression relative to the reference 5′ UTR. Most 5′ UTRs associated with substantial increases in expression had some attribute that precluded their broad utility. A1 emerged as a robust performer across ORFs and cell types that were tested. The A1 5′ UTR comprises a 76-nucleotide 5′ UTR sequence derived from the mouse Hist1h1b gene (SEQ ID NO: 1). This sequence is modified from the natural sequence by inclusion of a T7 transcription leader sequence. The A1 sequence was included in the screen based on: high ribosome density (by ribosome footprinting; mouse embryonic fibroblasts; Reid D W et al (2014) Cell 158(6):1362-74) high enrichment of small ribosomal subunits at the first AUG (by small subunit mapping); its moderate length and the absence of AUGs in the sequence.

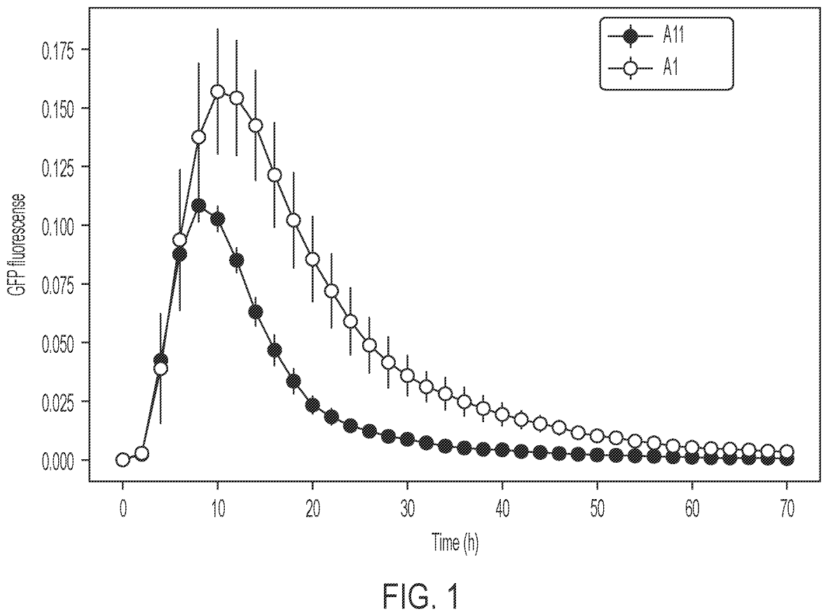

FIG. 1 shows protein expression from mRNA constructs having either a reference 5′ UTR or the A1 5′ UTR. Hela cells were transfected with mRNA constructs encoding a eGFP-degron having the specified 5′ UTRs. eGFP protein expression was evaluated by Imaging (Incucyte S3). The A1 5′ UTR resulted in about 2.4-fold increased protein expression compared to the construct having the reference 5′ UTR (Table 5). Based on the kinetics of eGFP fluorescence (as described in Mauger D M et al (2019) PNAS 116(48):24075-83) with the A1 5′ UTR, it was inferred that the corresponding mRNA had a half-life of about 6.9 hours compared to a half-life of about 1.9 hours with the construct containing the reference 5′ UTR. In summary, the A1 5′ UTR increased the expression of the protein encoded by the mRNA. In an embodiment, this effect is due to an increase in the half-life of the mRNA itself.

TABLE 5
Protein expression and mRNA half-life

| | Reference | |
|---|---|---|
| | 5′ UTR | |
| | (A11) | A1 |
| Total expression (AUC) | 1.0 | 2.4 |
| Translation efficiency | 1.0 | 1.1 |
| (inferred protein/mRNA/time) | | |
| mRNA half-life (inferred, in hours) | 1.9 | 6.9 |

### Example 2: In Vivo Increase in Protein Expression with LNP Formulated mRNA Having the A1 5′ UTR

This example describes an increase in protein expression in vivo with the A1 5′ UTR in a murine and rat animal model.

For evaluating the effect of A1 in vivo in mice, BALB/c mice were intravenously dosed with 0.5 mg/kg of LNP formulated ffLuc mRNA. The mRNA constructs either had the A11 reference 5′ UTR or the A1 5′ UTR. A cap comprising the sequence of GG was used. The animals were imaged at 6 hours and 48 hours post-dosing. There were 8 animals in each group.

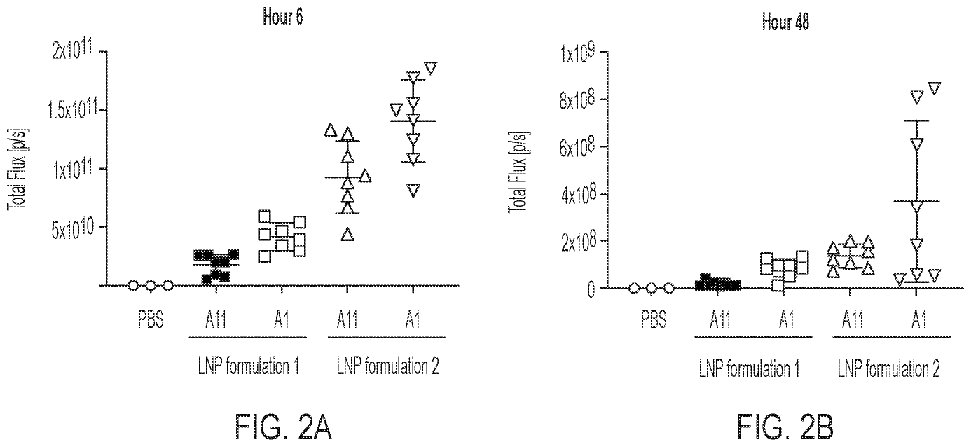

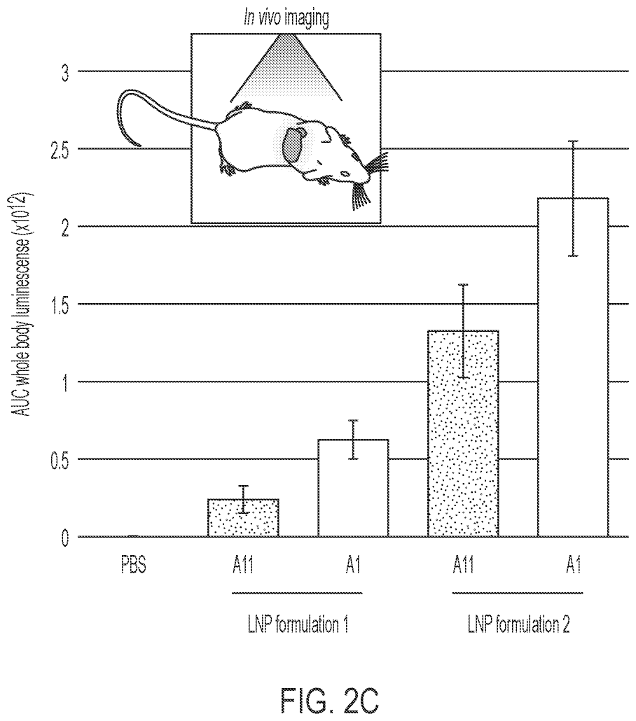

The data in FIGS. 2A-2C shows that A1 confers substantial increases (e.g., about 1.2-2-fold increase) in ffLuc activity in vivo in mice. FIG. 2A shows the total flux at 6 hours post administration and FIG. 2B shows the total flux at 48 hours post administration of the LNP. The impact was observed across both LNP formulations (each comprising an ionizable amino lipid embraced by Formula IC, a phospholipid, cholesterol and a PEG lipid) that were tested.

A similar experiment was performed to evaluate the effect of A1 in rats. Sprague-Dawley rats were intravenously dosed with 0.5 mg/kg of LNP formulated target mRNA. At the indicated time points, serum was obtained from the animals for evaluation of target protein levels. The level of target protein was assessed using ELISA. There were 11 mice in each group.

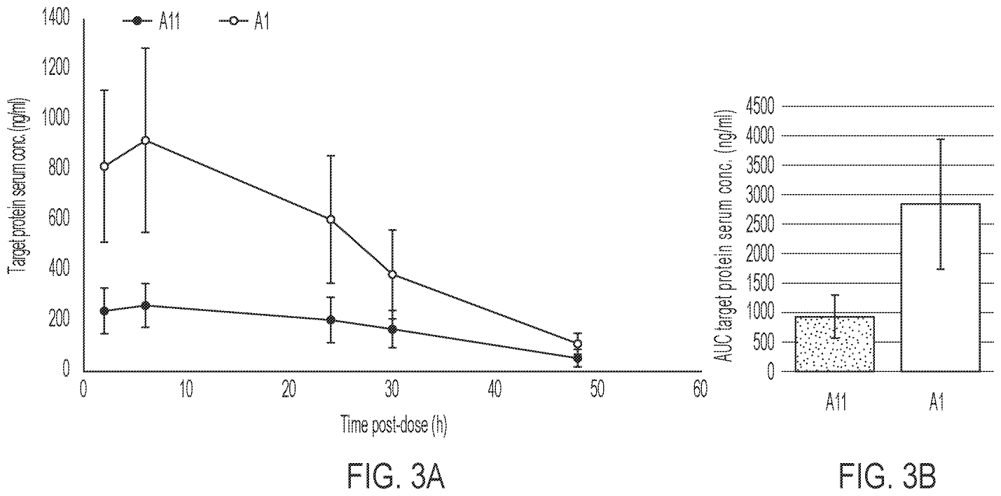

As shown in FIG. 3, the A1 5′ UTR conferred substantial increases (e.g., about 2.7-fold increase) in target expression in rat compared to the reference 5′ UTR.

### Example 3: Increased Target Protein Expression in Primary Hepatocytes with LNP Formulated mRNA Having the A1 5′ UTR

This Example describes increased expression of a target protein encoded by an mRNA having the A1 5′ UTR in rat, rhesus macaque and human primary hepatocytes.

Briefly, LNP formulated mRNA encoding a target protein was delivered HepatoPacs seeded with hepatocytes from rat, rhesus macaque, or human liver. The mRNA constructs either had the A11 reference 5′ UTR or the A1 5′ UTR. A cap comprising the sequence of GG was used. The cells were cultured in 1% matching serum and 200 ng of LNP formulated m RNA was delivered per well.

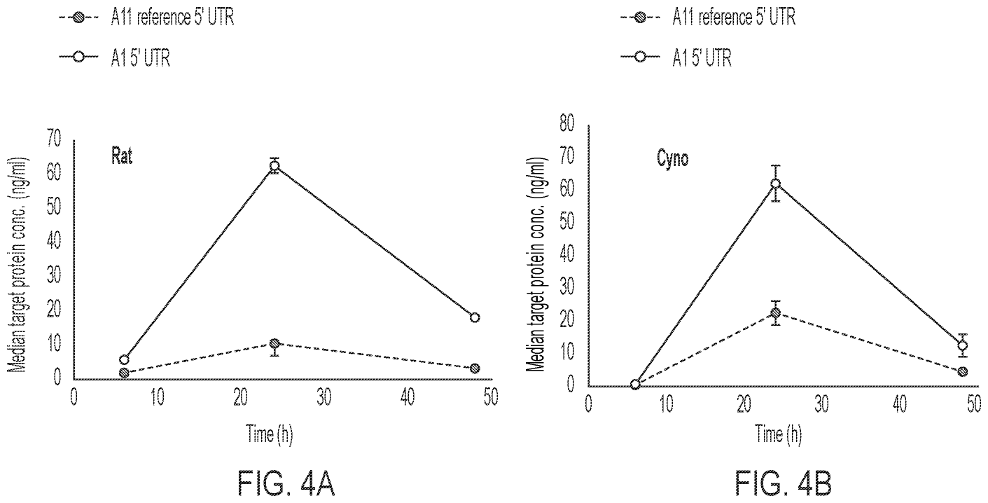

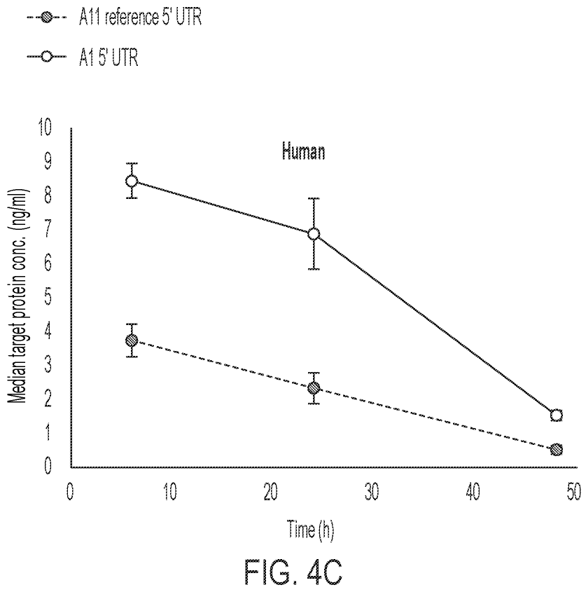

FIGS. 4A-4C show substantial increase in secreted target protein expression in cell medium in: rat (FIG. 4A), cyno (FIG. 4B) and human (FIG. 4C) primary hepatocytes with LNP formulated mRNA constructs having the A1 5′ UTR as compared to the reference 5′ UTR.

### Example 4: Increased Target Protein Expression in Mouse Immune Cells with LNP Formulated mRNA Having the A1 5′ UTR

This example describes increased expression of a target protein in various immune cell populations in vivo.

BALB/c mice were intravenously dosed with 0.5 mg/kg of LNP formulated mRNA encoding a target protein. The mRNA constructs either had the A11 reference 5′ UTR or a A1 5′ UTR. A cap comprising the sequence of GG was used. At 24 hours after administration, mice were sacrificed, and spleen and blood were evaluated by flow cytometry for target protein expression/level. There were 6 animals in each group.

The data is summarized in Tables 6-7. A1 was found to be consistently associated with increased expression of target protein expression in a broad array of mouse immune cells in spleen and blood. The magnitude of effect varied between cell types and the impacts on mean fluorescence intensity (MFI) were similar to percentage positive cells.

TABLE 6
Spleen (percent target protein+)

| | | | | | | | | Red pulp | |
|---|---|---|---|---|---|---|---|---|---|
| | | | | | | CD11b + | CD11b- | macs | CD11b + |
| | | | NKT | | Granulo- | CD11c + | CD11c + | CD11b- | F4/80 + |
| 5′UTR | B cells | T cells | cells | NK cells | cytes | cells | cells | F4/80hi | Macs |
| All | 14 | 12 | 20 | 14 | 27 | 66 | 73 | 97 | 57 |
| A1 | 19 | 15 | 28 | 25 | 40 | 73 | 77 | 99 | 75 |
| Fold | 1.4 | 1.2 | 1.4 | 1.8 | 1.5 | 1.1 | 1.1 | 1.0 | 1.3 |
| change | | | | | | | | | |

TABLE 7
Blood (percent target protein+)

| | | | | | | CD11b + | CD11b- | CD11b + |
|---|---|---|---|---|---|---|---|---|
| | | | | | Granulo- | CD11c + | CD11c + | F4/80 + |
| 5′UTR | B cells | T cells | NKT cells | NK cells | cytes | cells | cells | Macs |
| A11 | 4 | 3 | 5 | 13 | 7 | 54 | 14 | 56 |
| A1 | 6 | 5 | 8 | 19 | 20 | 64 | 25 | 72 |
| Fold | 1.5 | 1.5 | 1.6 | 1.4 | 3.0 | 1.2 | 1.8 | 1.3 |
| change | | | | | | | | |

### Example 5. Increased Target Protein Expression in Human PBMCs with LNP Formulated mRNA Having the A1 5′ UTR

This example describes increased expression of a target protein in human peripheral blood mononuclear cells (PBMC).

Human PBMCs were contacted with LNP formulated mRNA encoding a target protein. The mRNA constructs either had a reference 5′ UTR or the A1 5′ UTR. A cap comprising the sequence of GG was used. The PBMC cells were cultured in the presence of human serum. Cells from 3 donors were used in this experiment. After incubating with the LNPs for 24, 48, or 72 hours, the cells were harvested and processed for flow cytometry.

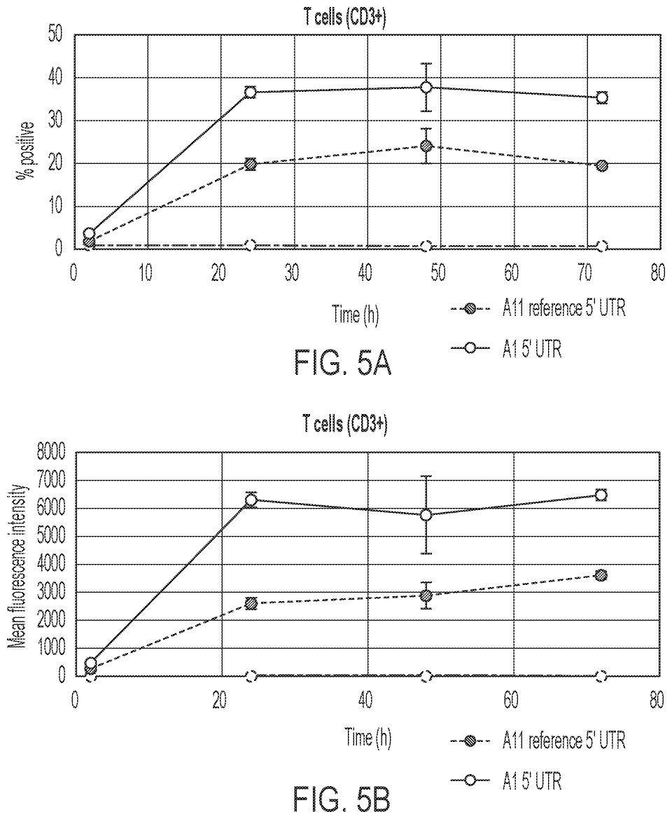

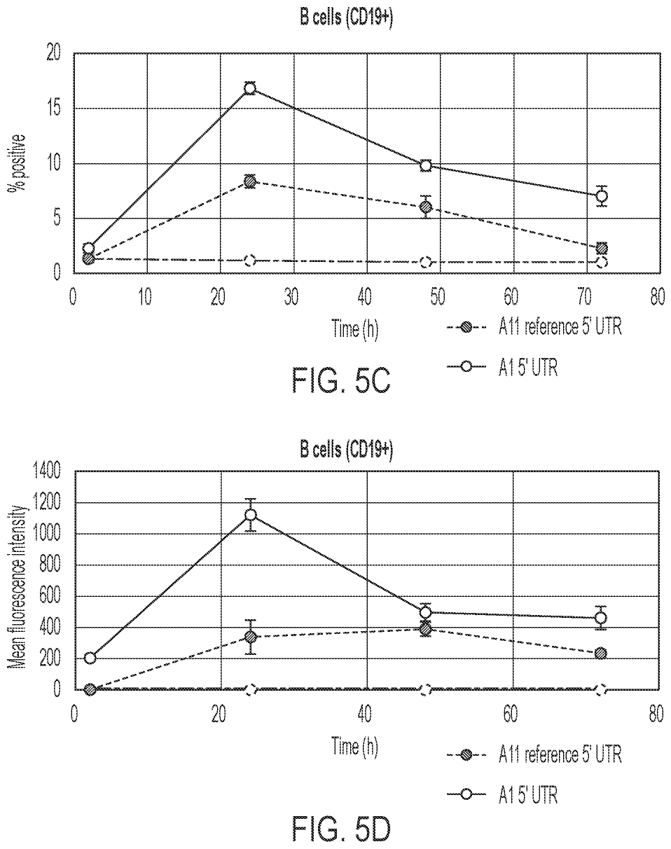

As shown in FIGS. 5A-5B, LNP formulated mRNA having A1 resulted in increased expression of the target protein in T cells as compared to the control. A similar effect was observed in B cells (FIGS. 5C-5D). This data shows that the presence of A1 in the mRNA construct results in increased expression of the encoded protein in human PBMCs.

### Example 6: In Vitro Effect of mRNA Having the A1 5′ UTR in a Rare Disease Model

This Example describes the level of a target protein associated with a rare disease in Hep3B cells. The target protein is encoded by an mRNA having the A1 5′ UTR or the A11 reference 5′ UTR.

For this experiment, Hep3B cells were transfected with mRNA constructs encoding the target protein. mRNA constructs comprising two versions of the ORF sequence encoding the target protein were used. The mRNA constructs had the A1 5′ UTR or a reference 5′ UTR. A cap comprising the sequence of GG was used. The cells were cultured in DMEM supplemented with 10% fetal bovine serum at 37° C. 24 hours post transfection, the cells were harvested and processed for Western blotting.

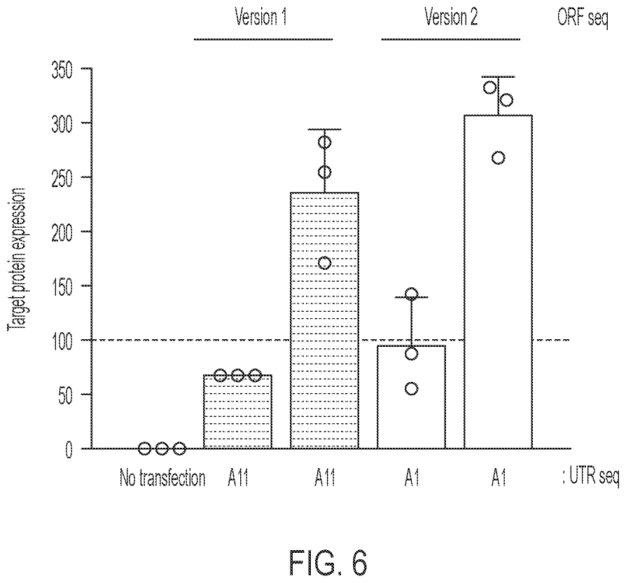

As shown in FIG. 6 mRNA constructs having either versions of the ORF sequence with the A1 5′ UTR resulted in increased expression of the encoded target protein associated with a rare disease.

### Example 7. Protein Expression from A1 Variants

This Example describes protein expression from constructs having different A1 5′ UTR sequences. mRNA sequences encoding a green fluoresce protein were generated with the following 5′ UTR sequences: A1, A2 or A3. A cap comprising the sequence of GG was used. mRNAs were transfected into Hep3B cells using Lipofectamine 2000 and fluorescence was monitored using an IncuCyte. Total fluorescent signal was summed over a time course.

The results are provided in Table 8. All mRNA sequences encoding a green fluoresce protein showed similar levels of protein expression. The data shows that the A1 5′ UTR and its two derivatives confer similar levels of protein expression.

TABLE 8

| 5′ UTR | Greencyto_Hep3b_AUC |
|---|---|
| A3 | 1.25 |
| A2 | 1.23 |
| A1 | 1.22 |

### Example 8: In Vivo Protein Expression from A1 Variants

This Example describes in vivo protein expression from a construct having a modified A1 5′ UTR sequence.

Mice were dosed with mRNA bearing a reference 5′ UTR or a modified A1 sequence bearing additional uridines and an improved Kozak sequence. A cap comprising the sequence of GG was used. Expression of the encoded protein in the liver was measured by immunoblot at 2 days and 8 days post-dosing. Expression was normalized to a loading control and to endogenous expression of the encoded protein.

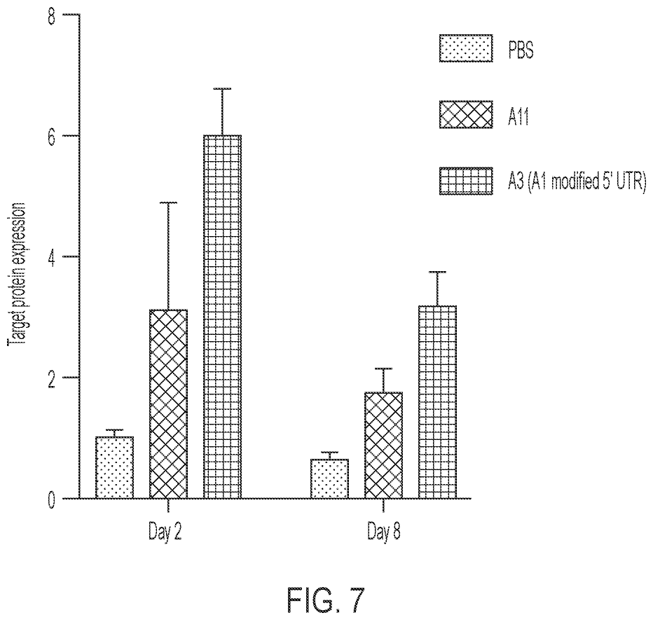

The results of this experiment are shown in FIG. 7. In vivo protein expression from the construct having the modified A1 5′ UTR was higher on days 2 and 8 post-dosing compared to the control.

### Example 9: Discovery and Application of 3′ UTR Sequences for Extension of mRNA Half-Life

This Example describes a 3′ UTR screen to identify 3′ UTRs which could confer an increase in mRNA half-life.

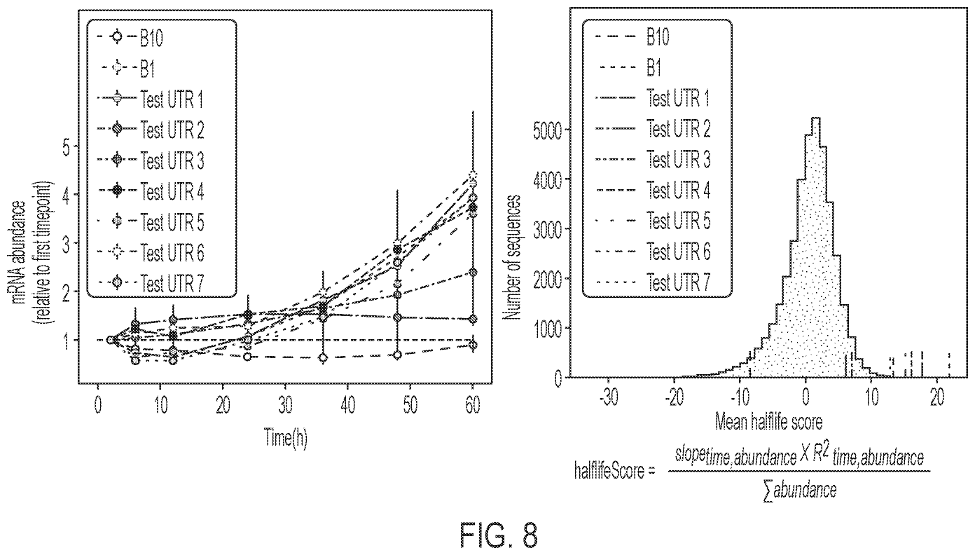

mRNA pools with 120,000 unique 3′ UTR sequences for each of 3 ORFs: GFP-degron (cytoplasmic), GLuc (secreted), and membrane protein were generated. To identify 3′ UTRs that may confer increased mRNA half-life, these pools were electroporated into Hep3b, HeLa, AML-12, and JAWSII cells, then the relative abundance of each 3′ UTR sequence was assessed by amplicon sequencing over a time course. In this experiment, an increase in the relative abundance of a 3′ UTR sequence may indicate increased mRNA half-life, and is quantified as shown (FIG. 8).

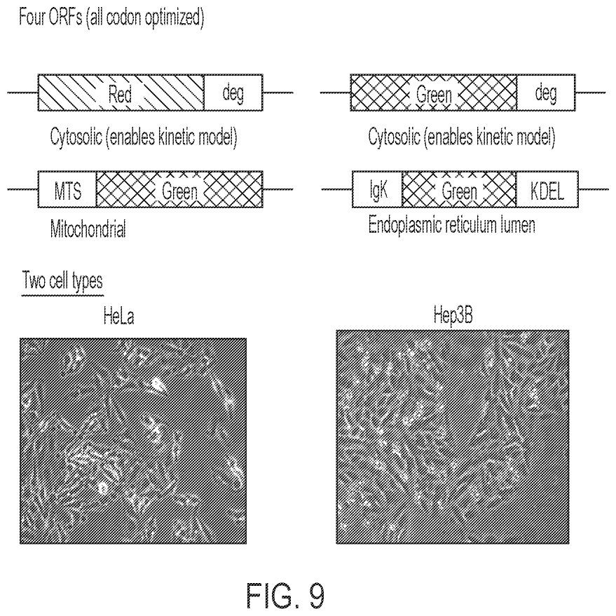

Several 3′ UTR sequences associated with high half-life scores were incorporated into a 3′ UTR screen, which assessed the performance of several classes of 3′ UTR sequences using fluorescent reporters (FIG. 9). In this screen, computational modeling (Mauger D M et al (2019) PNAS 116(48):24075-83) was used to infer translational efficiency and mRNA half-life associated with each mRNA. Performance of 3′ UTRs was fairly consistent across ORFs (R=0.4) and cell types (R=0.7).

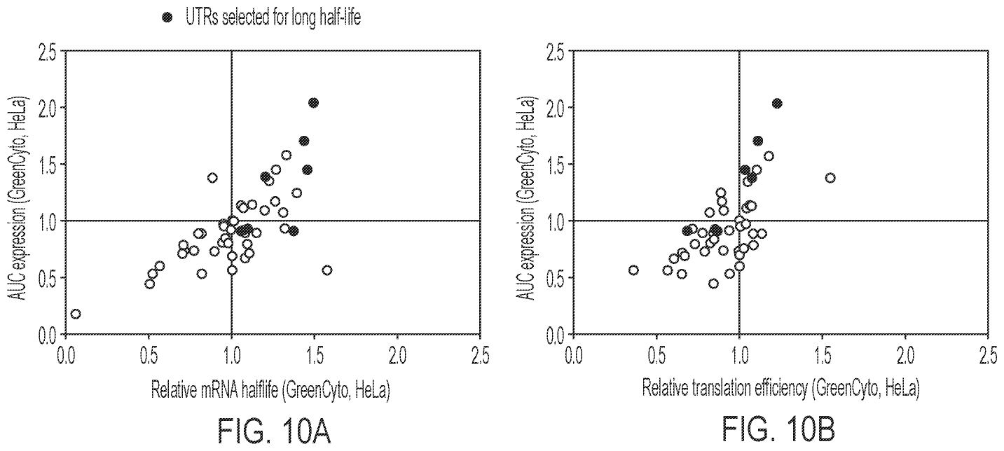

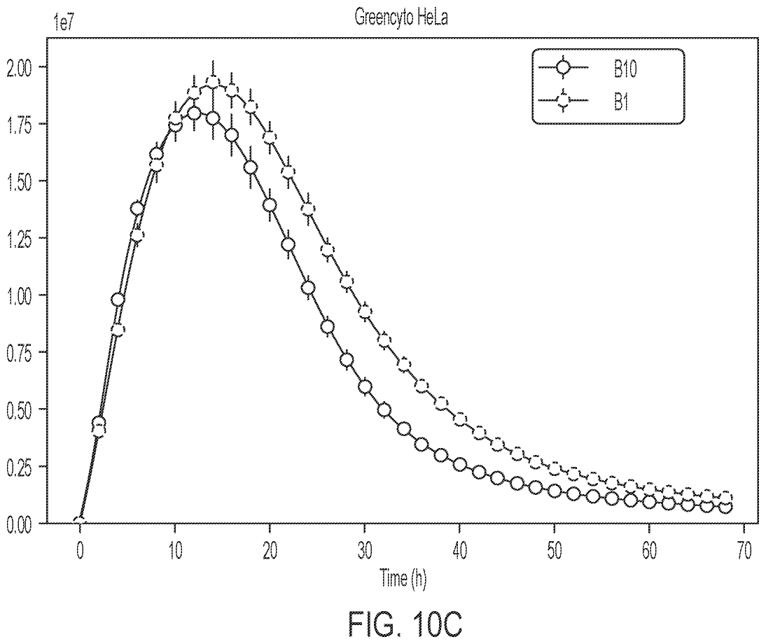

According to the computational model, each of the 3′ UTR sequences selected from the high-throughput half-life screen was associated with increased mRNA half-life relative to the reference 3′ UTR (FIGS. 10A-10C). However, the effect sizes were small—the very highest half-lives observed were only 50% increased relative to the reference (FIG. 10A). Translation efficiency also varied among these 3′ UTR sequences, with the highest overall expression conferred by mRNAs that had high inferred half-life and translation efficiency (FIG. 10A-10B).

The sequence most consistently associated with extended half-life is derived from the B1 gene; an example of the expression profile is shown (FIG. 10C). Interestingly, the natural B1 mRNA does not itself have an unusually high half-life (t½=5.5h; Tani H et al (2012) Genome Research 22(5):947-956). This sequence also had the highest half-life score in the high-throughput 3′ UTR screen. Based on these observations, the B1 3′ UTR was used in subsequent studies to further evaluate its effects on mRNA half-life and protein expression.

### Example 10: Engineering of Stop Elements

This Example describes the engineering of stop elements comprising consecutive stop codons.

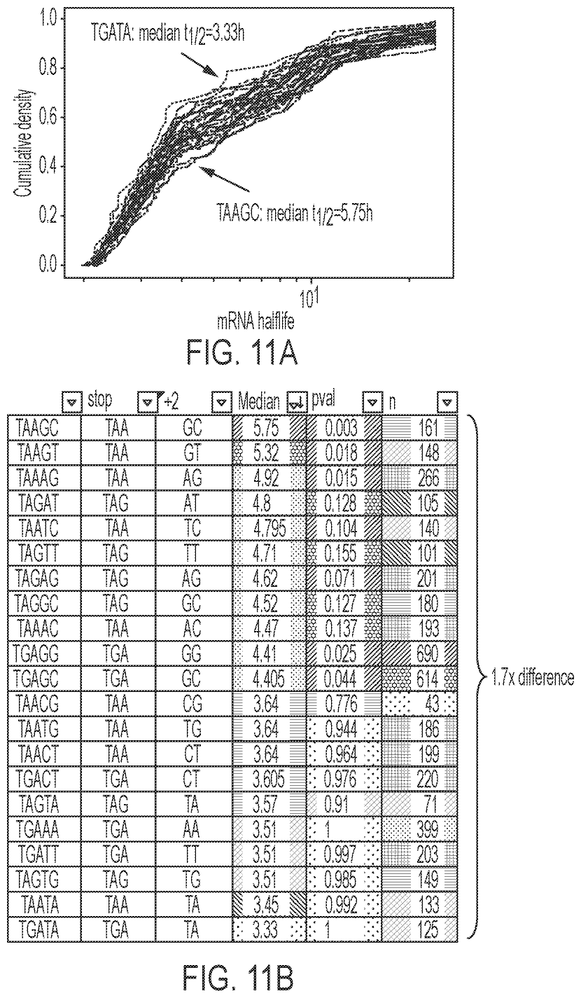

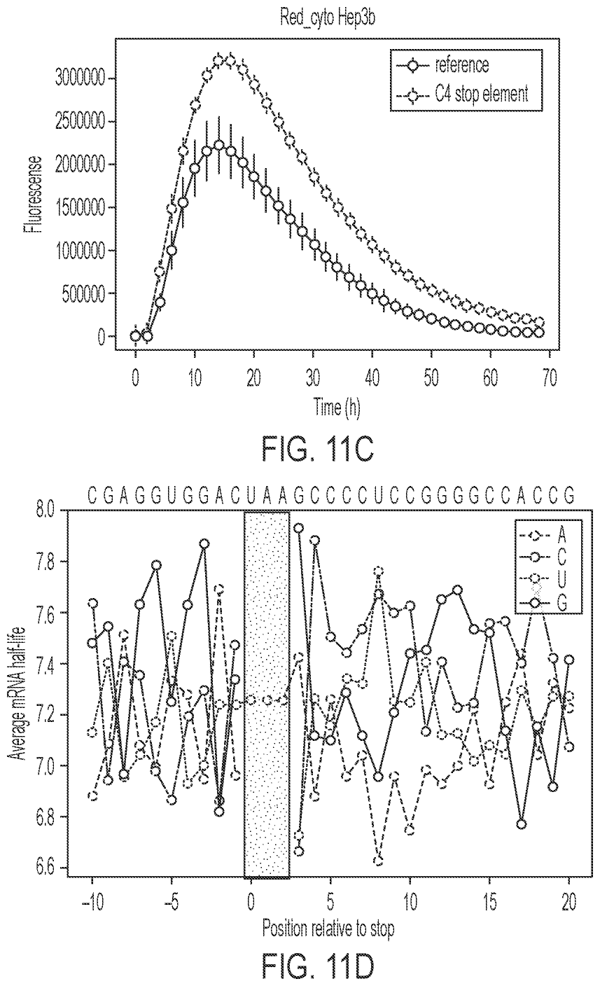

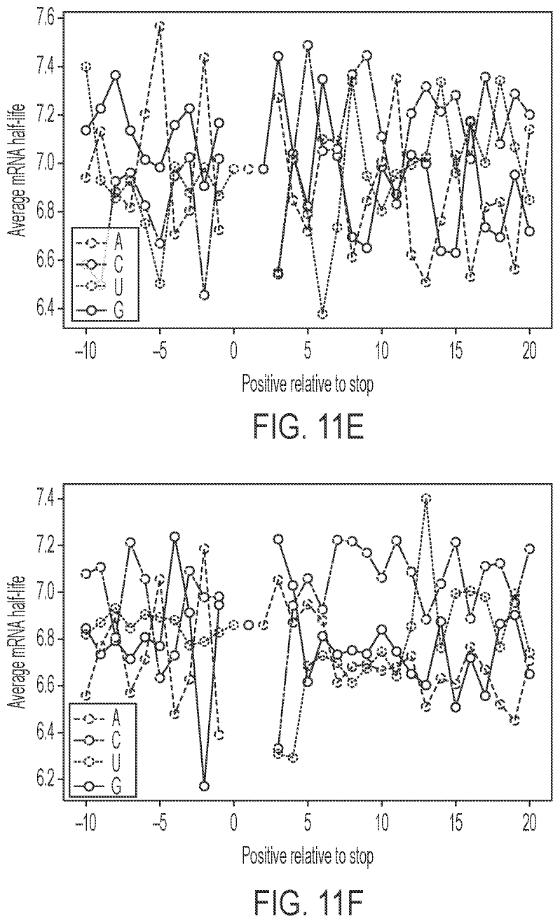

Given the substantial literature related to stop codon context and its impact on stop codon function (reviewed in Dabrowski M. et al, (2015) RNA Biol 12(9):950-958), it was hypothesized that stop codon cassettes—defined as a stop codon and its nucleotide context—may also impact mRNA half-life. To assess this possibility, the median mRNA half-life (Tani H et al (2012) Genome Research 22(5):947-956) for mRNAs bearing different stop elements was calculated. There were substantial and significant differences in median half-lives among mRNAs with different stop elements, with those related to consecutive stop codons being associated with the lowest median half-lives (FIGS. 11A-11B). Based on these data, several new stop codon elements were tested in the fluorescent reporters from the 3′ UTR screen described in Example 9.

Across several reporters and cell types, modest (˜1.5×) but consistent increased in overall expression that were driven by increases in mRNA half-life according to computational modelling was observed (example shown in FIG. 11C). These new stop elements are outlined in Table 9. Also included in this table are stop codon cassettes derived from a separate bioinformatic analysis where single nucleotides at each position relative to the stop codon were separately assessed for correlation with mRNA half-life (C7, C9; FIG. 11D-11F).

TABLE 9
Exemplary stop elements

| SEQ ID | Sequence | |
|---|---|---|
| NO | information | Sequence |
| 26 | C1 | UGAUAAUAG |
| 27 | C2 | UAAUAGUAA |
| 28 | C3 | UAAGUCUAA |
| 29 | C4 | UAAAGCUAA |
| 30 | C5 | UAAGUCUCC |
| 31 | C6 | UAAGGCUAA |
| 32 | C7 | UAAGCCCCUCCGGGG |
| 33 | C8 | UAAAGCUCCCCGGGG |
| 34 | C9 | UAAGCCCCU |
| 35 | C10 | UAAAGCUCC |
| 36 | C11 | UAGGGUUAA |

### Example 11: Increased Expression of a Target Protein Encoded by mRNA Having Different Stop Elements

This Example describes increased protein expression of a target encoded by an mRNA having different stop elements. The target protein used in this example is associated with a rare disease. The stop elements used in this example are C5, C4, C11, C3, a reference stop element (C1) and a control. All stop elements were incorporated into an mRNA with the target protein open reading frame and transfected into HepG2 cells. Target protein levels were assessed by immunoblot at the indicated time points.

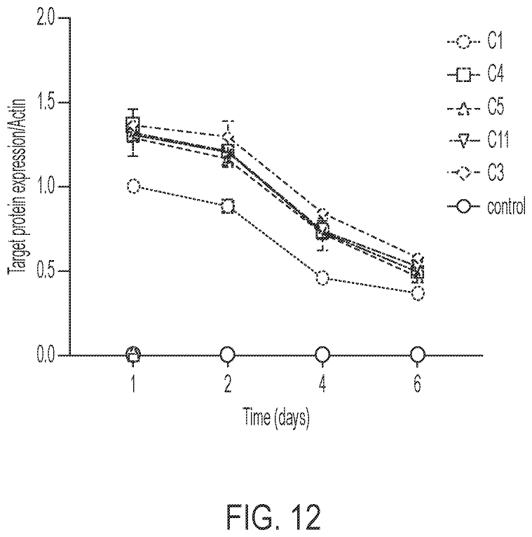

As shown in FIG. 12, in HepG2 cells each test stop element was associated with a moderate increase in protein levels in vitro relative to the reference stop element.

### Example 12: In Vivo Expression of a Target Protein Encoded by mRNA Having Different Stop Elements

This Example describes increased protein expression of a target encoded by an mRNA having different stop elements. The target protein used in this example is associated with a rare disease. Briefly, mice were dosed with mRNA bearing a triple stop element (alpha) or a modified stop element indicated in the legend. Expression of the encoded protein in liver was measured by immunoblot at 2 days and 8 days post-dose, normalized to a loading control and to endogenous expression of the encoded protein. The stop elements used in this example are C7, C10, C8, or a reference stop element.

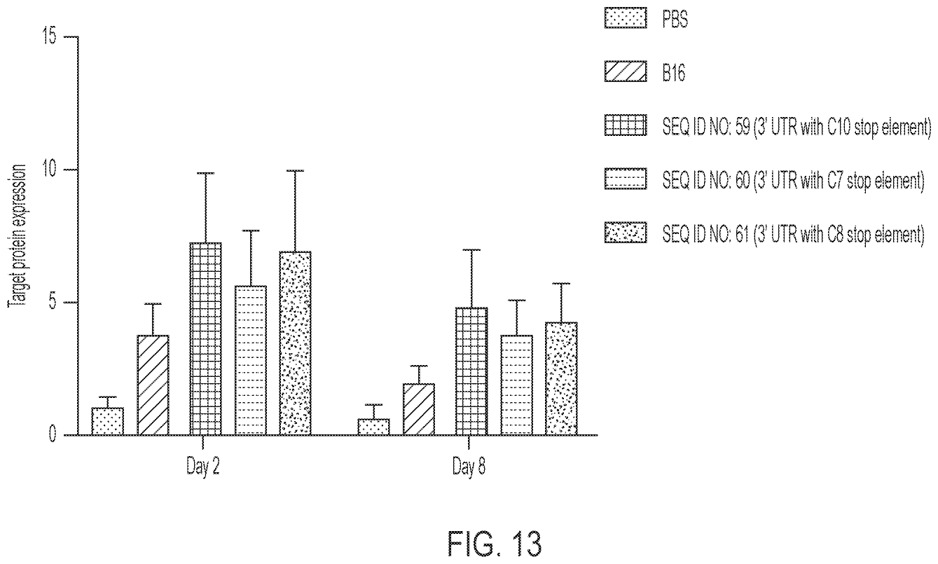

As shown in FIG. 13, in mouse liver, each test stop element was associated with an in vivo increase in protein levels on days 2 and 8 relative to the reference stop element.

### Example 13: Expression of a Reporter Protein Encoded by mRNAs Having Different Stop Elements

This example describes the expression of a green fluorescent protein encoded by mRNA constructs having a sequence encoding the green fluoresce protein and different stop elements.

Hep3B cells were transfected (by Lipofectamine 2000) with mRNA constructs having a reference stop element, a C6 stop element, a C4 stop element or a C3 stop element and fluorescence was monitored hourly by Incucyte, with the final expression value being the sum of fluorescence over all time points. Table 10 shows the results of this experiment. All values are relative to the reference stop element.

TABLE 10

| | Green expression |
|---|---|
| | (Hep3B cells) |
| reference | 1.00 |
| C6 | 1.02 |
| C4 | 1.44 |
| C3 | 1.58 |

Expression of the fluorescent reporter protein was increased with all stop elements tested, with the highest expression from mRNA constructs having the C4 or C3 stop elements.

### Example 14. Stable Tail and Protein Variant Expression

A target protein was expressed in each of full length and truncated forms and screened for expression in vitro at 24 and 96 hours. Select mRNA constructs were modified by ligation to stabilize the poly(A) tail. Ligation was performed using 0.5-1.5 mg/mL mRNA (5′ Cap1, 3′ A100), 50 mM Tris-HCl pH 7.5, 10 mM MgCl₂, 1 mM TCEP, 1000 units/mL T4 RNA Ligase 1, 1 mM ATP, 20% w/v polyethylene glycol 8000, and 5:1 molar ratio of modifying oligo to mRNA. Modifying oligo has a sequence of 5′-phosphate-AAAAAAAAAAAAAAAAAAAA-(inverted deoxythymidine (idT)) (SEQ ID NO; 128) (see below). Ligation reactions were mixed and incubated at room temperature (˜22° C.) for 4 h. Stable tail mRNA were purified by dT purification, reverse phase purification, hydroxyapatite purification, ultrafiltration into water, and sterile filtration. Ligation efficiency for each mRNA was >80% as assessed by UPLC separation of ligated and unligated mRNA. The resulting stable tail-containing mRNAs contained the following structure at the 3′ end, starting with the polyA region: A₁₀₀-UCUAGAAAAAAAAAAAAAAAAAAAA (SEQ ID NO: 124)-inverted deoxythymidine.

Modifying oligo to stabilize tail (5′-phosphate-AAAAAAAAAAAAAAAAAAAA-(inverted deoxythymidine (SEQ ID NO: 128)):

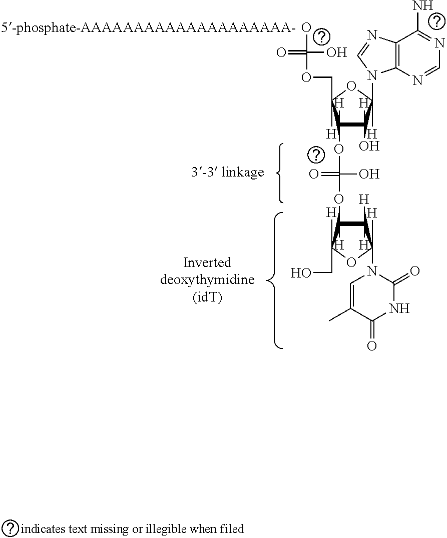

Each of the target protein encoding mRNA constructs were transfected at a concentration of 0.1 ug/mL and protein expression was examined at 24 and 96 hours post-transfection and compared to expression resulting from transfection of a control.

### Example 15: In Vivo Effect of LNP Formulated mRNA Encoding an Immune Checkpoint Protein Having mRNA Elements Disclosed Herein

This Example describes the in vivo expression of an immune checkpoint protein encoded by an mRNA construct having a 5′ UTR and/or stabilized tail disclosed herein.

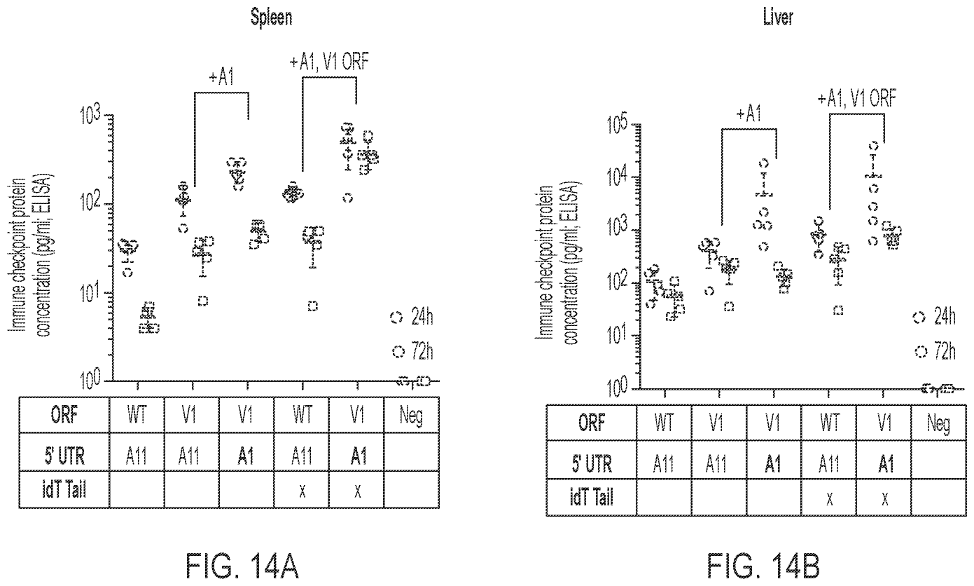

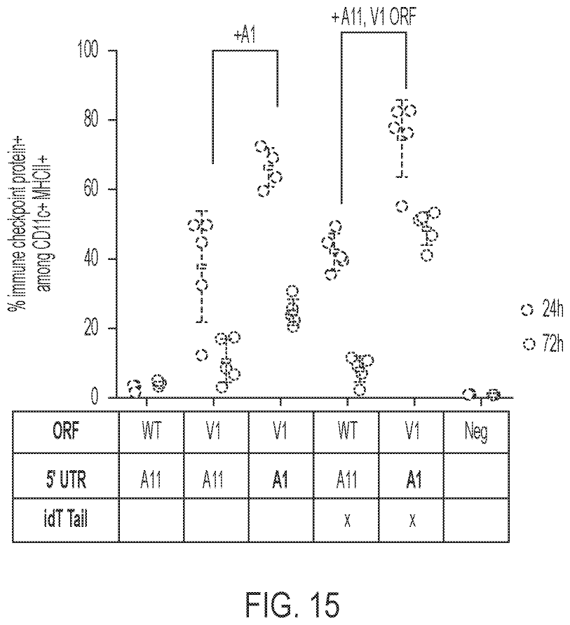

Briefly, mice were intravenously injected with 0.5 mg/kg of LNP formulated mRNAs encoding the immune checkpoint protein and having the mRNA elements specified in FIGS. 14A-14B. For this experiment, the ORF encoding the immune checkpoint protein was either the WT ORF or a variant thereof (v1). As controls, mRNAs having either a reference 5′ UTR or no stabilized tail were used. 24-hours and 72-hours post-dosing, mice were sacrificed and the spleen and livers were processed for evaluation of protein level by ELISA. A cap comprising the sequence of GG was used.

FIGS. 14A-14B depict the results of this experiment. Increased expression of the immune checkpoint protein was observed in the spleen (FIG. 14A) and liver (FIG. 14B) of mice dosed with LNP formulated mRNA having the A1 5′ UTR alone, the stabilized tail alone or both. Mice dosed with mRNA having both the A1 5′ UTR and the stabilized tail, showed a further increase in protein expression. In the spleen, the increased protein expression was observed at both time-points. In the liver, the effect was more pronounced at the 24-hour time point.

Taken together, this data shows that the A1 5′ UTR results in increased in vivo expression of the immune checkpoint protein. The data also supports a synergy between the A1 5′ UTR and stabilized tail in further increasing protein expression.

### Example 16: In Vivo Expression of an Immune Checkpoint Protein Encoded by mRNA Having Various mRNA Elements in Dendritic Cells

This Example describes the expression of an immune checkpoint protein in dendritic cells from mice which were administered LNP formulated mRNA encoding the immune checkpoint protein and having a 5′ UTR and/or stabilized tail disclosed herein.

Briefly, mice were intravenously injected with 0.5 mg/kg of LNP formulated mRNAs encoding the immune checkpoint protein and having the mRNA elements specified in FIG. 15. For this experiment, the ORF encoding the immune checkpoint protein was either the WT ORF or a variant thereof. As controls, mRNAs having either a reference 5′ UTR or no stabilized tail were used. 24-hours and 72-hours post-dosing, mice were sacrificed, and dendritic cells were isolated for flow cytometry analysis. A cap comprising the sequence of GG was used.

As shown in FIG. 15, increased expression of the immune checkpoint protein was observed at 24- and 72-hour post dosing in mice administered with LNP formulated mRNA having the A1 5′ UTR alone, the stabilized tail alone or both. Dendritic cells from mice dosed with mRNA having both the A1 5′ UTR and the stabilized tail, showed a further increase in protein expression.

In summary, this data shows that the A1 5′ UTR results in increased expression of an immune checkpoint protein in murine dendritic cells compared to the control. The data also supports a synergy between the A1 5′ UTR and stabilized tail in further increasing protein expression.

### Example 17. In Vivo Effect of mRNA Having the A1 5′ UTR (Together with a Cap Comprising the Sequence GA) and/or the B1 3′ UTR

This Example describes in vivo assessments of firefly luciferase luminescence and/or a target protein encoded by mRNAs having the A1 5′ UTR (together with a cap comprising the sequence GA), a 3′ UTR comprising B1 (“B1 3′ UTR), or both.

For this experiment, mice were co-dosed with mRNAs encoding the target protein and firefly luciferase. The mRNA constructs either had the A1 5′ UTR (together with a cap comprising the sequence GA), a B1 3′ UTR, or both or reference UTRs. At 24 hours, serum was collected and analyzed for target protein level by ELISA. Liver and spleen were subsequently dissected and analyzed for luminescence.

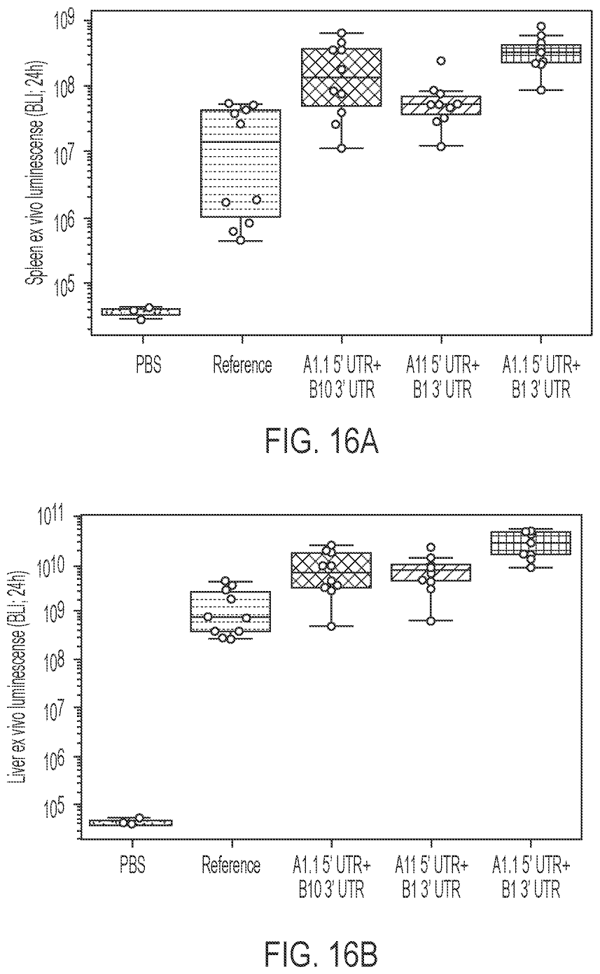

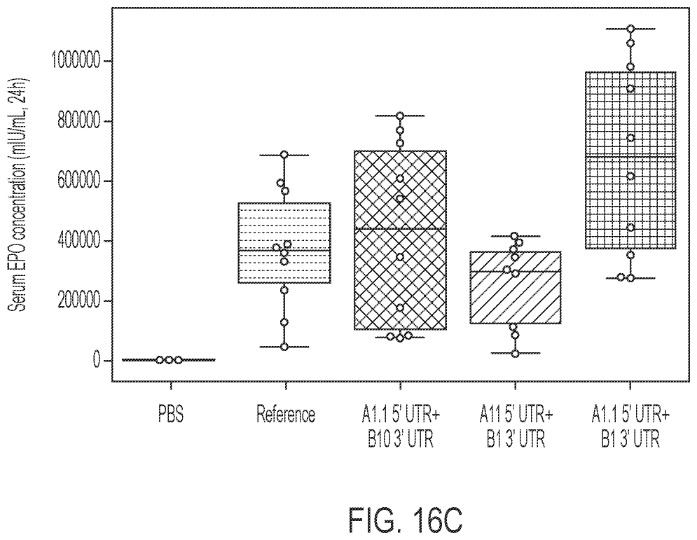

The results of this experiment are shown in FIGS. 16A-16C. The spleen and liver of mice administered mRNAs having either the A1 5′ UTR (together with a cap comprising the sequence GA), a B1 3′ UTR, or both showed increased luminescence compared to the control. The mRNA construct having both the A1 5′ UTR (together with a cap comprising the sequence GA) and the B1 3′ UTR resulted in highest luminescence in the spleen (FIG. 16A) and liver (FIG. 16B), supporting an additive effect of both elements. A similar effect was observed for target protein expression in serum (FIG. 16C).

### Example 18: Effect of LNP Formulated mRNA Having Various mRNA Elements in a Pulmonary Disease Model

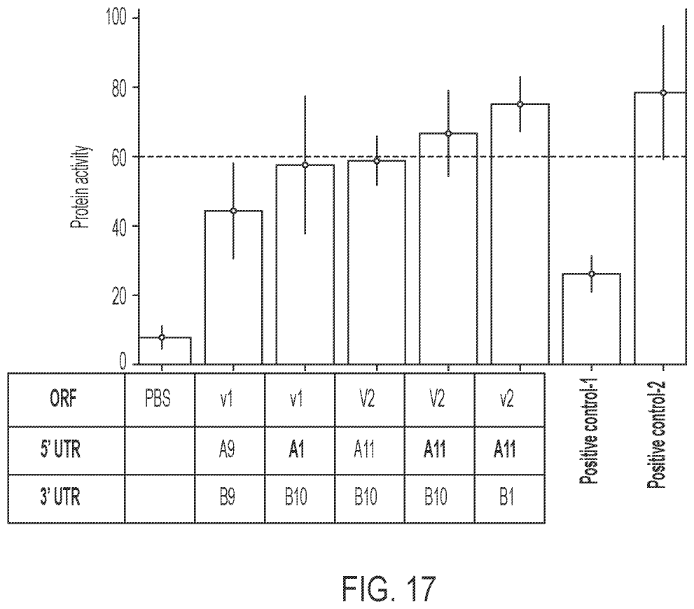

This Example describes the activity of a target protein in human bronchial epithelial cells, which target protein is encoded by an mRNA having various elements shown in FIG. 17.

Briefly, LNP formulated mRNAs encoding the target protein and having the mRNA elements specified, were dosed onto human CF bronchial epithelial cells. Two different ORFs encoding the target protein were used in this experiment: v1 and v2. Eighteen hours post-dosing, the activity of the target protein was measured. As controls, cells were treated with two standard of care drugs: positive control 1 and positive control 2. A cap comprising the sequence of GG was used in this Example.

As shown in FIG. 17, the mRNA construct having a 3′ UTR comprising B1 (“B1 3′ UTR) and A1 5′ UTR and resulted in an increase in target protein function compared to the controls. An increase in target protein function was also observed with the mRNA construct having the A1 5′ UTR. Taken together, this data demonstrates the combined effect of the A1 5′ UTR and B1 3′ UTR in increasing target protein expression.

### Example 19: Effect of Stop Codon Readthrough in mRNA Having Different Stop Elements

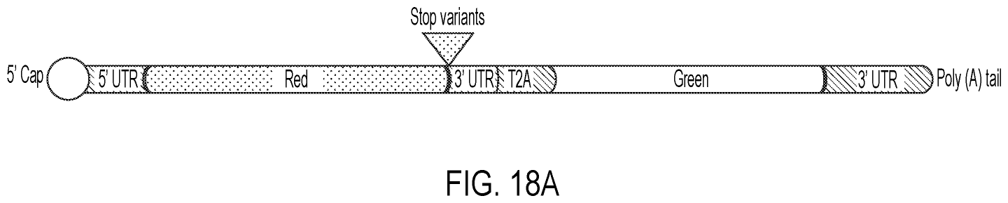

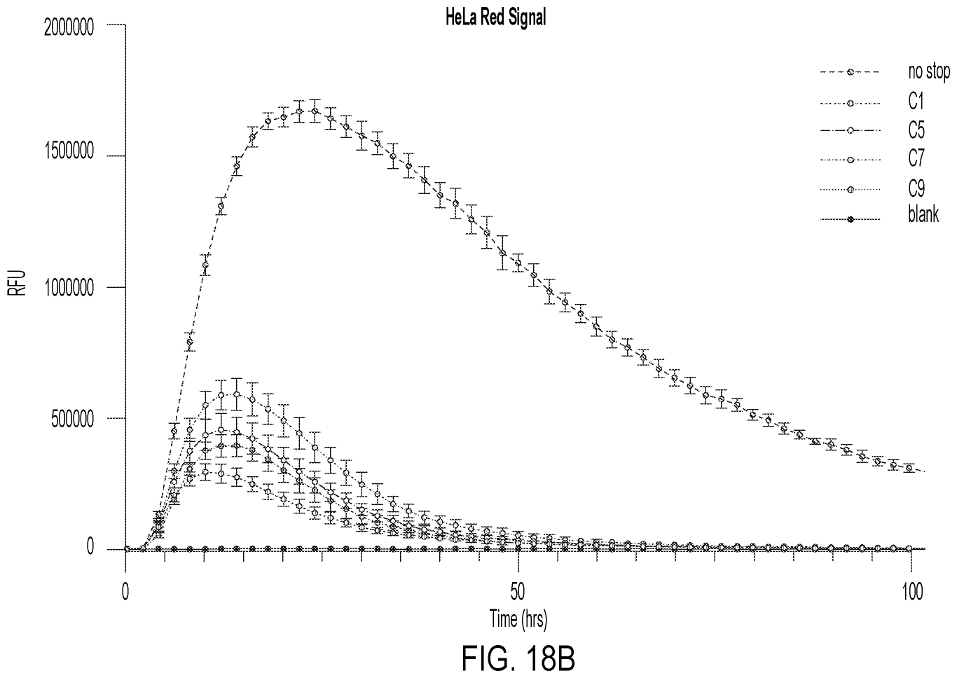

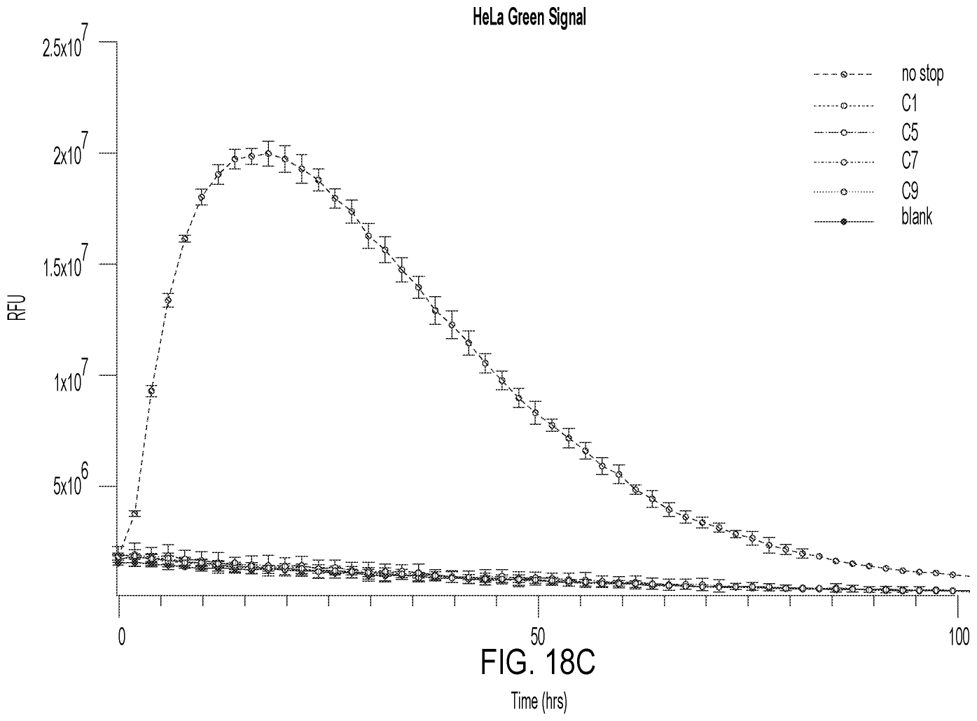

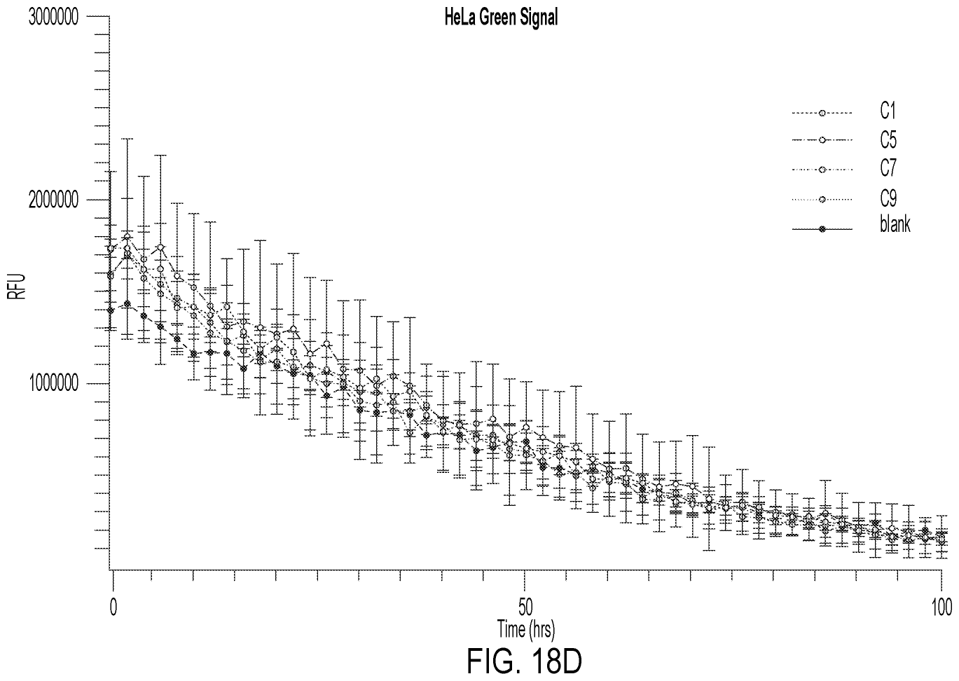

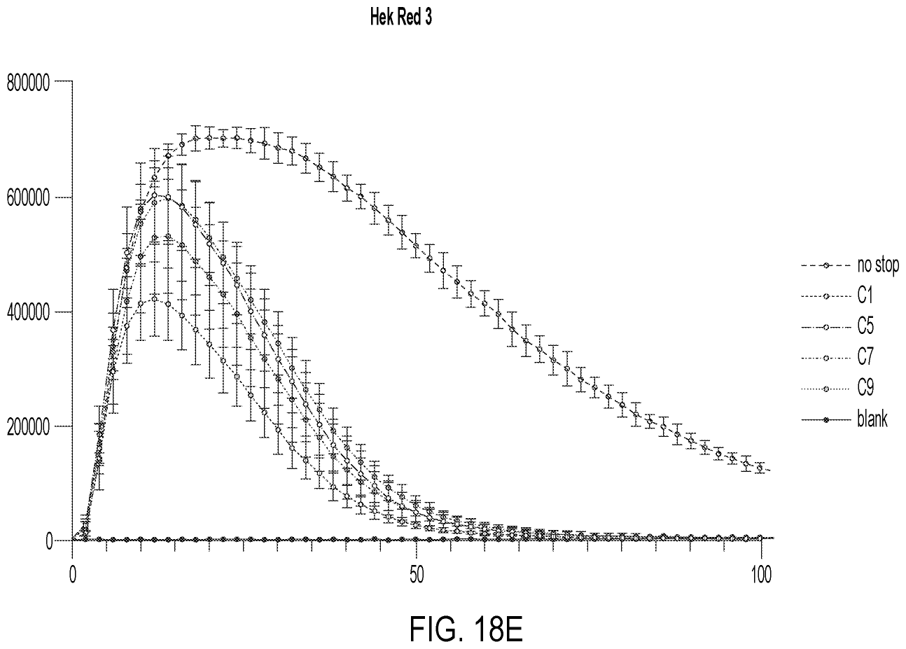

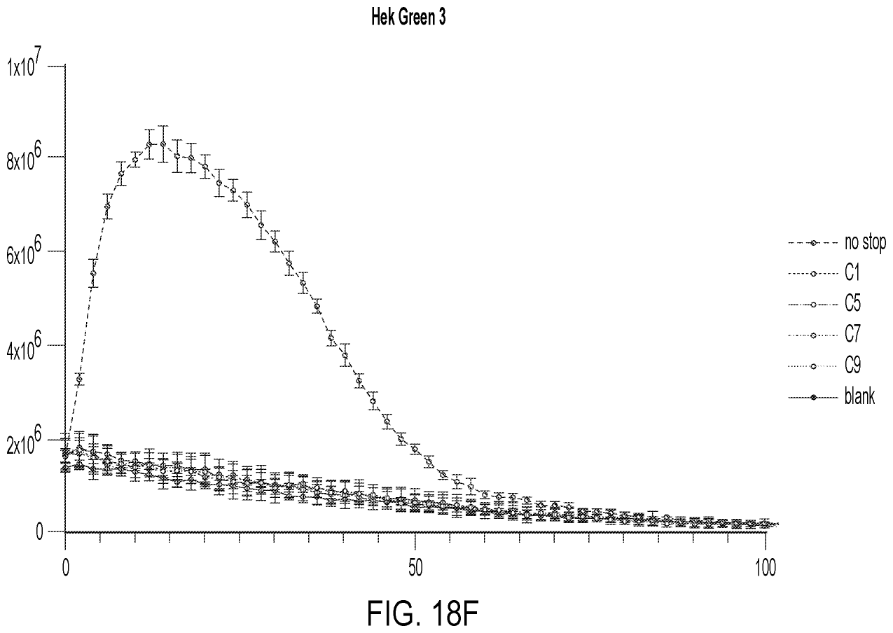

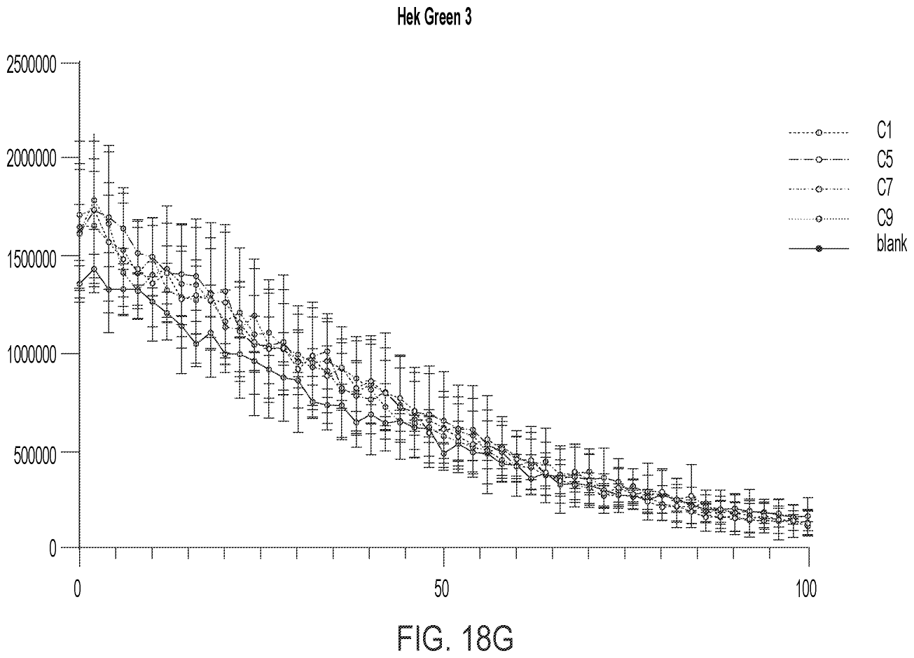

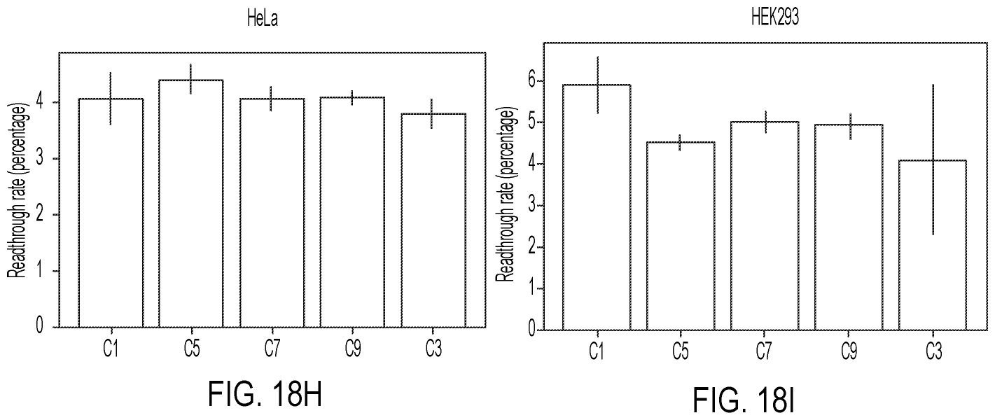

This Example describes the effect of stop codon readthrough in mRNA having different stop elements. The design of the mRNA constructs is shown in FIG. 18A. The stop elements used in this example are C1 (reference), C5, C7, C9, and C3. The mRNA constructs have a red fluorescence protein open reading frame and a green fluorescence protein open reading frame, separated by 3′ UTR and a sequence encoding T2A. All stop elements tested were incorporated into the red fluorescence protein open reading frame. An mRNA construct that does not have a stop codon incorporated into the red fluorescence protein open reading frame was used as a control. The mRNA constructs were transfected into HeLa cells and HEK293 by Lipofectamine 2000. The red and green fluorescence signals generated from the expression of the red and green fluorescence proteins, respectively, were monitored by real-time Incucyte.

As shown in FIG. 18B, the red fluorescence protein was expressed at comparable levels in Hela cells for all stop elements tested, whereas as shown in FIGS. 18C-18D, the expression of the green fluorescence protein was substantially low in Hela cells. Similarly, as shown in FIG. 18E, the red fluorescence protein was expressed at comparable levels in HEK293 cells for all stop elements tested, whereas as shown in FIGS. 18F-18G, the expression of the green fluorescence protein was substantially low in HEK293 cells. The estimates of readthrough rate in HeLa and HEK293 cells are shown in FIGS. 1811-18G, respectively. The lack of green fluorescence signal detected over background in any of the mRNA constructs having a stop element tested indicates no evidence of stop codon readthrough.

### Example 20: Expression of a Target Protein Encoded by mRNA Having Different Stop Elements

This Example describes increased protein expression of a target protein encoded by an mRNA having different stop elements. The target protein used in this example is human erythropoietin (hEPO). The stop elements used in this example are C1 (reference), C5, C10, C7, C8, and C9. All stop elements were incorporated into an mRNA with the target protein open reading frame and transfected into HeLa cells and HEK293 cells. Target protein levels were assessed by ELISA at 24- and 48-hours post-transfection.

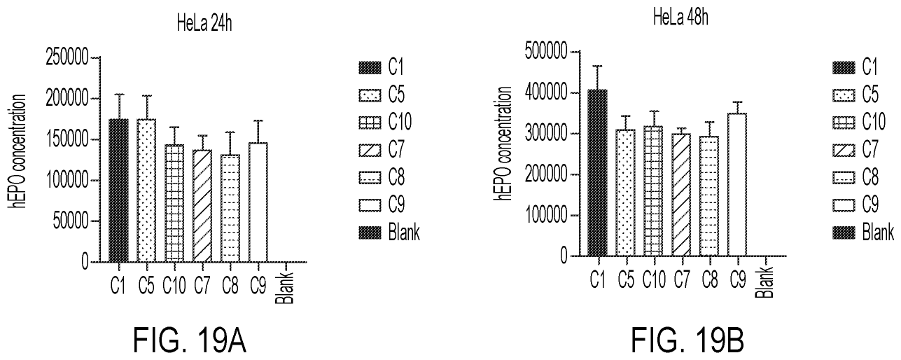

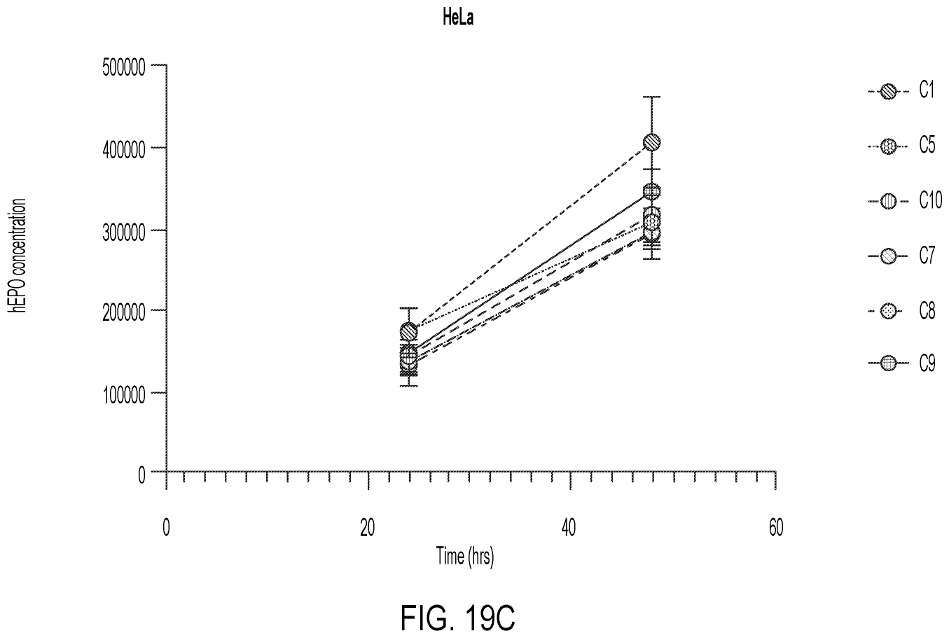

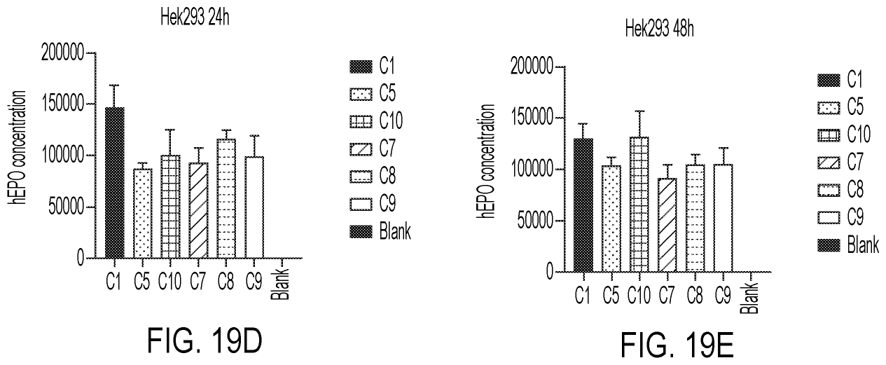

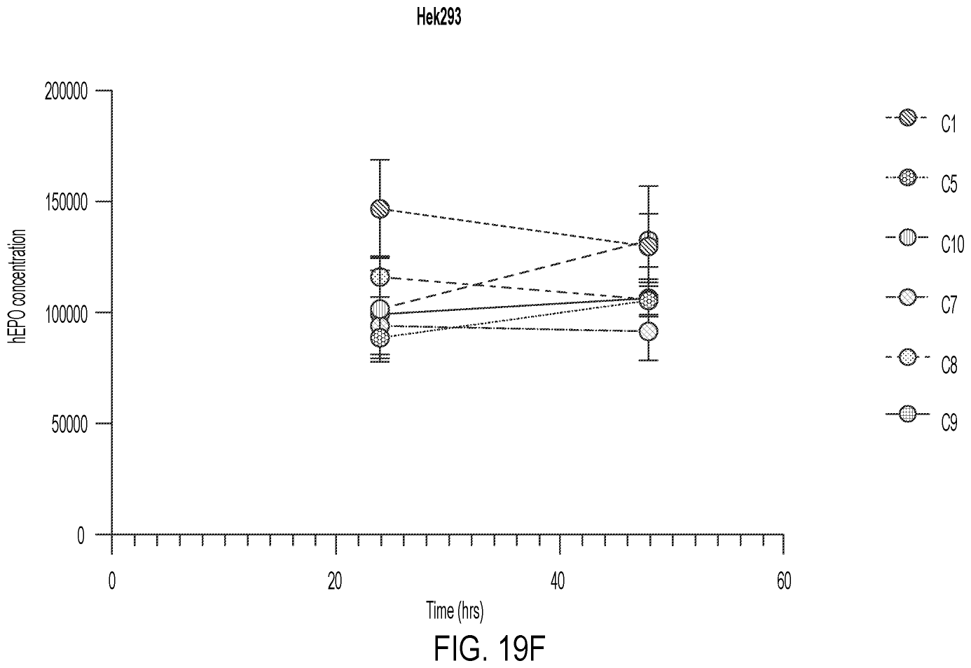

As shown in FIGS. 19A-19C, in HeLa cells each test stop element was associated with comparable protein levels in vitro relative to the reference stop element. Similarly, as shown in FIGS. 19D-19F, in HEK293 cells each test stop element was associated with comparable protein levels in vitro relative to the reference stop element.

### Example 21: Expression of a Target Protein Encoded by mRNA Having Different Stop Elements

This Example describes increased protein expression of a target protein encoded by an mRNA having different stop elements. The target protein used in this example is firefly luciferase (ffLuc). The stop elements used in this example are C1 (reference), C5, C10, C7, C8 and C9. All stop elements were incorporated into an mRNA with the target protein open reading frame and transfected into HeLa cells and Hep3b cells. Target protein levels were assessed by a luciferase assay at 24 and 48 hours post-transfection.

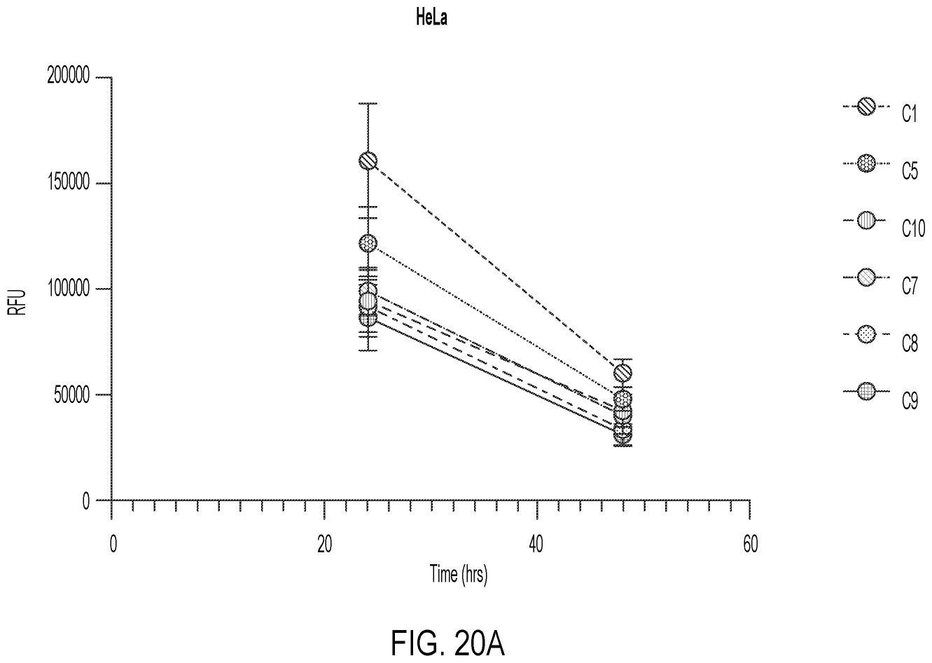

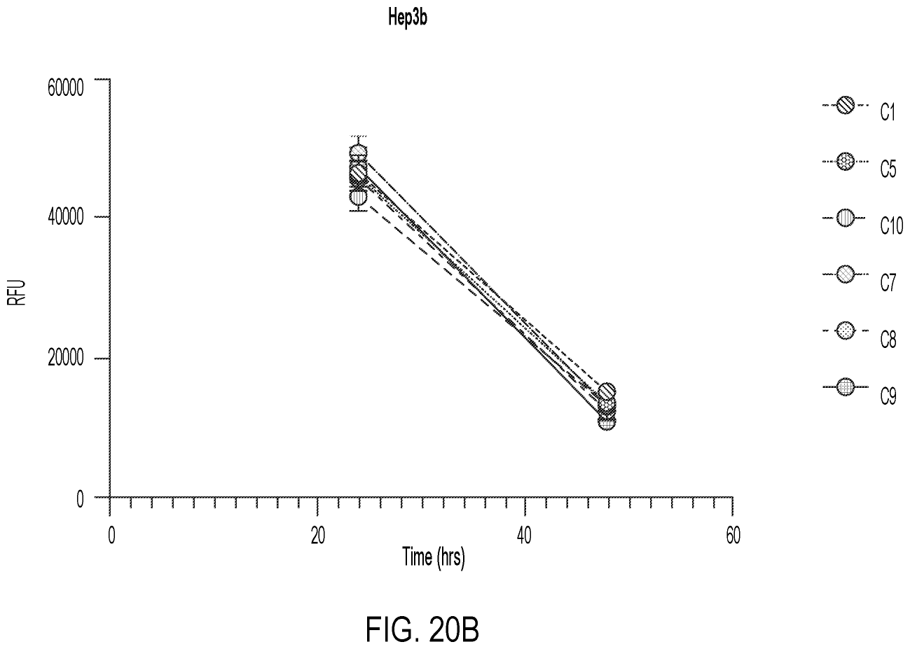

As shown in FIG. 20A, in HeLa cells each test stop element was associated with comparable protein levels in vitro relative to the reference stop element. Similarly, as shown in FIG. 20B, in Hep3b cells each test stop element was associated with comparable protein levels in vitro relative to the reference stop element.

### Example 22: In Vivo Expression of a Target Protein Encoded by mRNA Having Different Stop Elements

This Example describes increased protein expression of a target encoded by an mRNA having a stop element disclosed herein.

The target proteins used in this example are firefly luciferase (ffLuc) and human erythropoietin (hEPO). Briefly, mice were intravenously dosed with 0.5 mg/kg LNP formulated mRNA bearing a tested stop element. The stop elements used in this example are C1 (reference), C5, C10, C7, C8 and C9. All stop elements were incorporated into an mRNA with the target protein open reading frame. Expression of the encoded ffLuc protein was measured by whole-body luminescence imaging at 24 hours post-dose. Expression of the encoded hEPO protein was measured by ELISA at 1, 3, and 7 days post-dose.

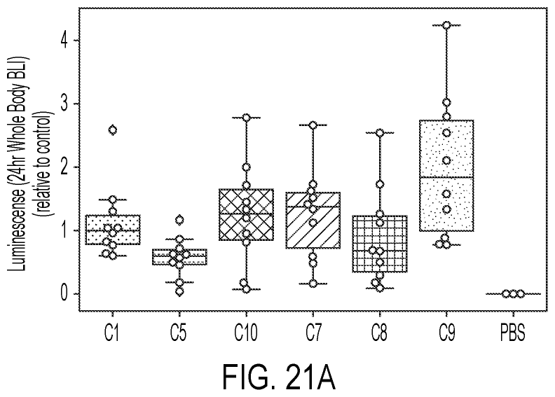

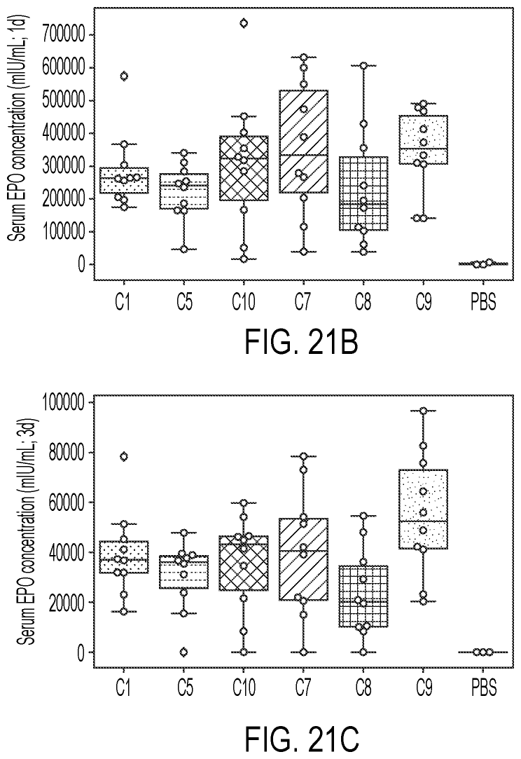

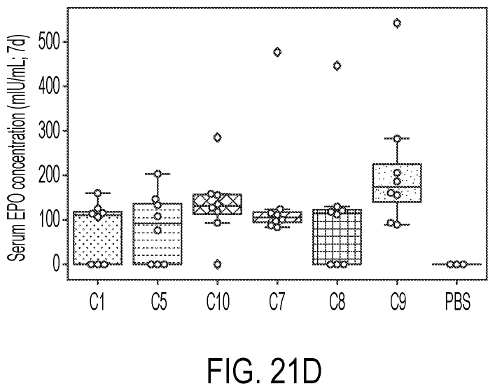

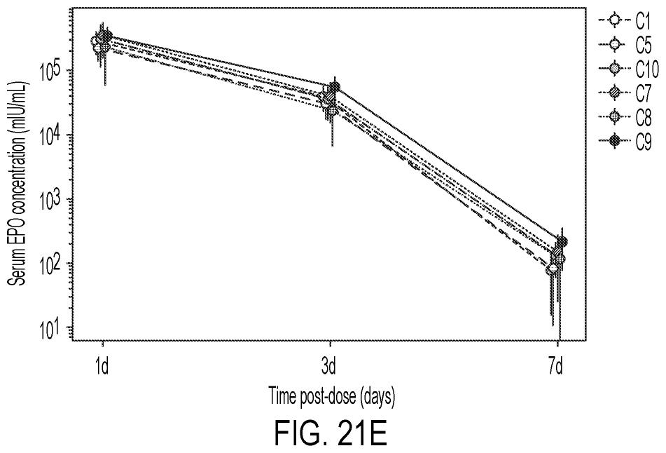

As shown in FIG. 21A, each test stop element was associated with comparable or increased ffLuc protein levels in vivo at 24 hours relative to the reference stop element. As shown in FIGS. 21B-21E, each test stop element was associated with comparable or increased serum hEPO protein levels in vivo at 1, 3, and 7 days relative to the reference stop element.

### Example 23: In Vivo Expression of a Target Protein Encoded by mRNA Having Different Stop Elements

This Example describes increased protein expression of a target encoded by an mRNA having a stop element disclosed herein.

The target proteins used in this example are firefly luciferase (ffLuc) and human erythropoietin (hEPO). The stop elements used in this example are C1 (reference), C10, C7, C8 and C9. All stop elements were incorporated into an mRNA with the target protein open reading frame. The 3′ UTR used for hEPO contains 3× mIR-142 binding sites.

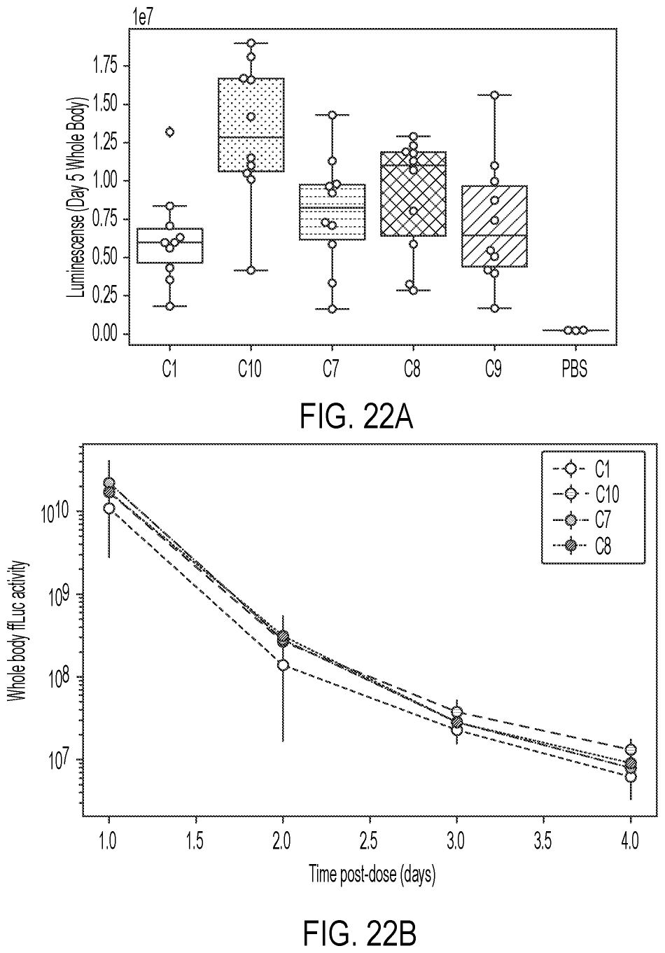

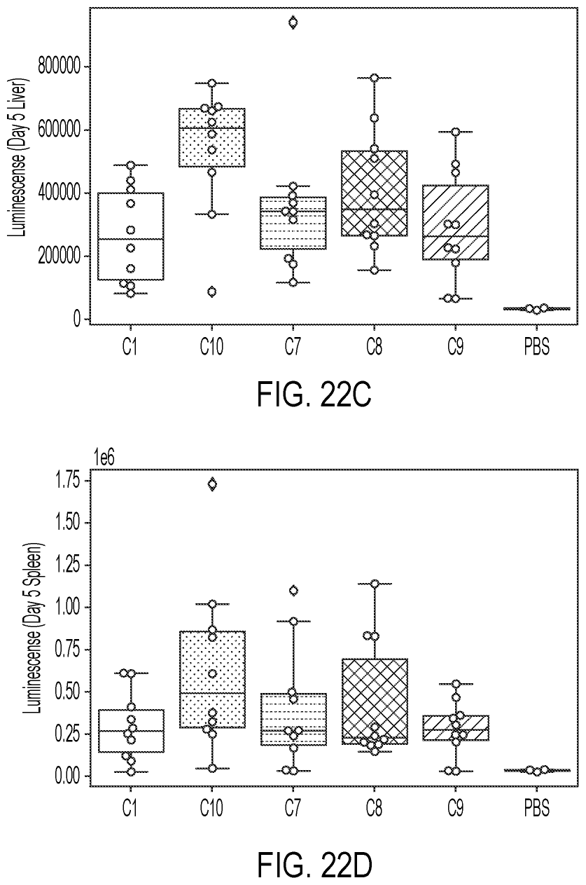

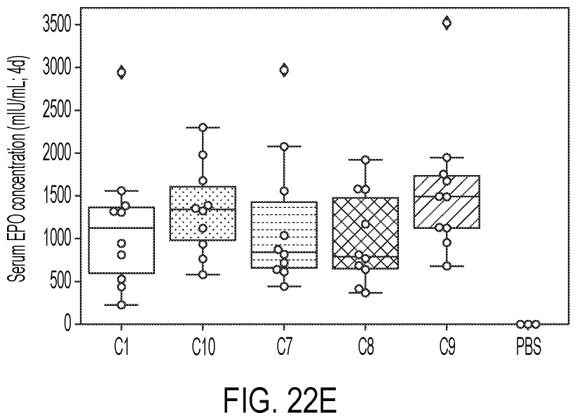

Briefly, mice were intravenously dosed with 0.25 mg/kg LNP formulated mRNA bearing a tested stop element indicated in FIGS. 22A-22E. Expression of the encoded ffLuc protein was measured by whole-body luminescence imaging at 1, 2, 3, 4, and 5 days post-dose and ex vivo (spleen and liver) luminescence imaging at 5 days post-dose. Expression of the encoded hEPO protein was measured by ELISA at 4 days post-dose.

As shown in FIGS. 22A-22B, each test stop element was associated with comparable or increased ffLuc protein levels in vivo at 1, 2, 3, 4, and 5 days post-dose relative to the reference stop element. As shown in FIGS. 22C-22D, each test stop element was associated with comparable or increased ffLuc protein levels ex vivo in liver and spleen at 5 days post-dose relative to the reference stop element. As shown in FIG. 22E, each test stop element was associated with comparable or increased serum hEPO protein levels in vivo at 4 days relative to the reference stop element.

### Example 24: Expression of a Target Protein Encoded by mRNA Having Different Stop Elements

This Example describes increased protein expression of a target protein encoded by an mRNA having different stop elements. The target protein used in this example is firefly luciferase (ffLuc). The stop elements used in this example are C1 (reference), C5, C10, C7, C8 and C9. All stop elements were incorporated into an mRNA with the target protein open reading frame. The LNP formulated mRNAs were transfected into hepatocyte islands co-cultured with 3T3 fibroblasts or 3T3 fibroblasts alone. Target protein levels were assessed by a luciferase assay at 24, 72, and 96 hours post-transfection.

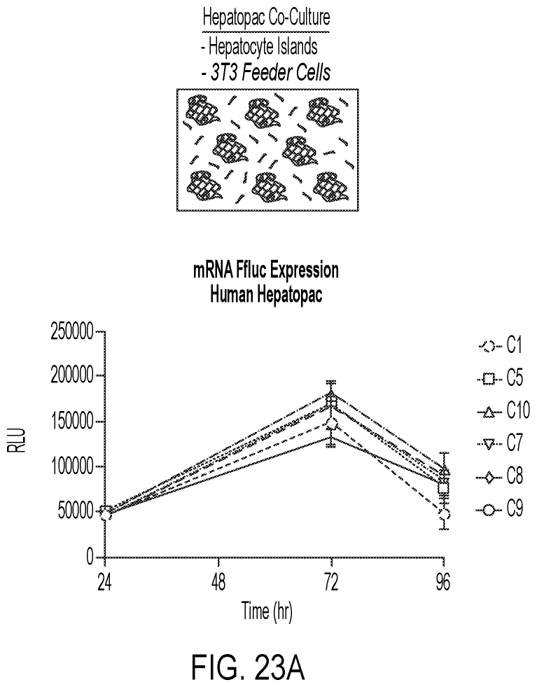

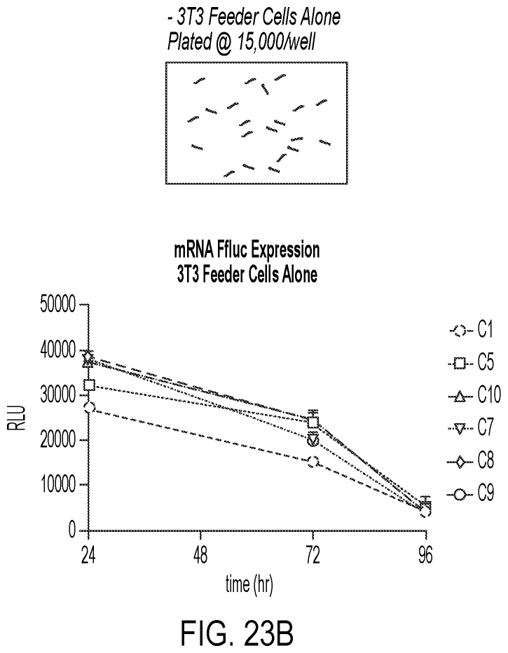

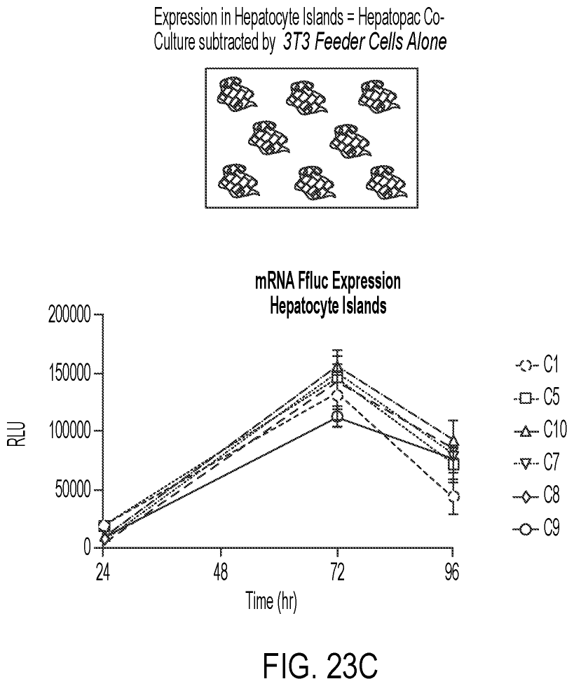

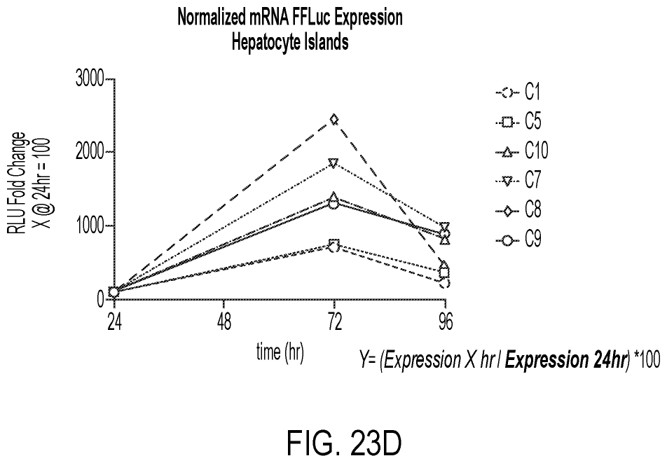

As shown in FIGS. 23A-23D, each test stop element was associated with comparable or increased protein levels in hepatocyte islands in vitro relative to the reference stop element.

### Example 25: Expression of a Target Protein Encoded by mRNA Having Different Stop Elements

This Example describes increased protein expression of a target protein encoded by an mRNA having different stop elements. The target protein used in this example is human erythropoietin (hEPO). The stop elements used in this example are C1 (reference), C5, C10, C7, C8 and C9. All stop elements were incorporated into an mRNA with the target protein open reading frame. The LNP formulated mRNAs were transfected into hepatocyte islands co-cultured with 3T3 fibroblasts or 3T3 fibroblasts alone. Target protein levels were assessed by luciferase assay at 24, 72, and 96 hours post-transfection.

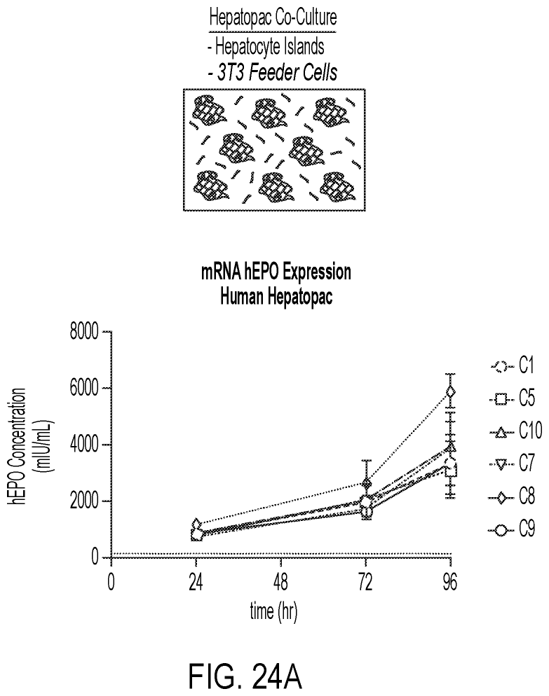

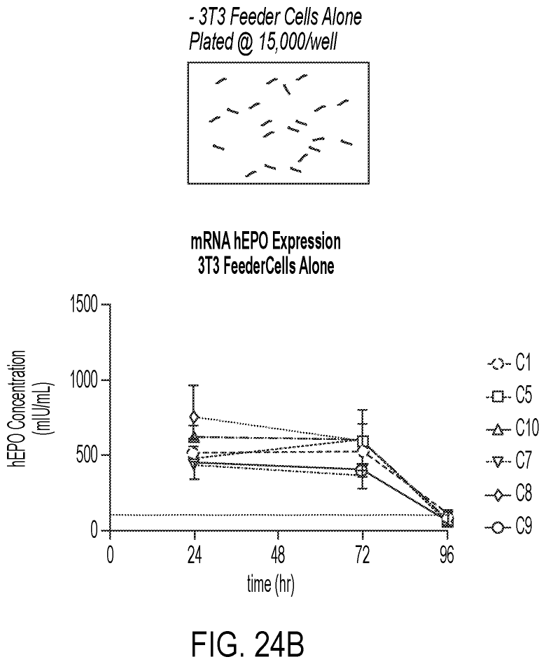

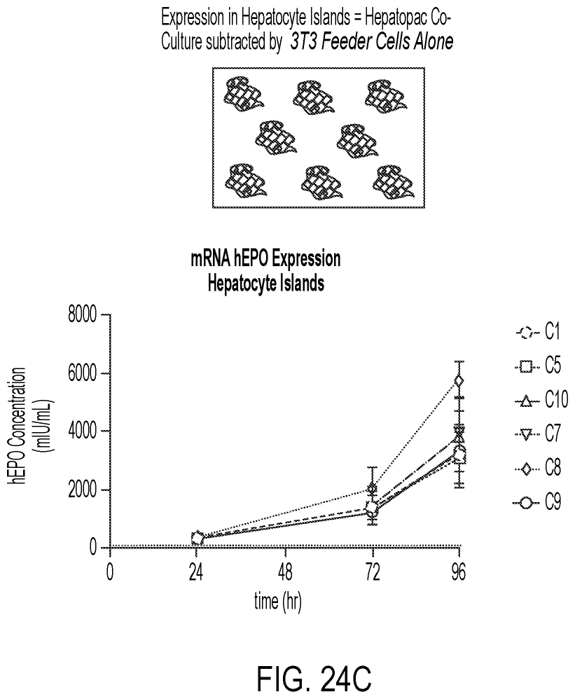

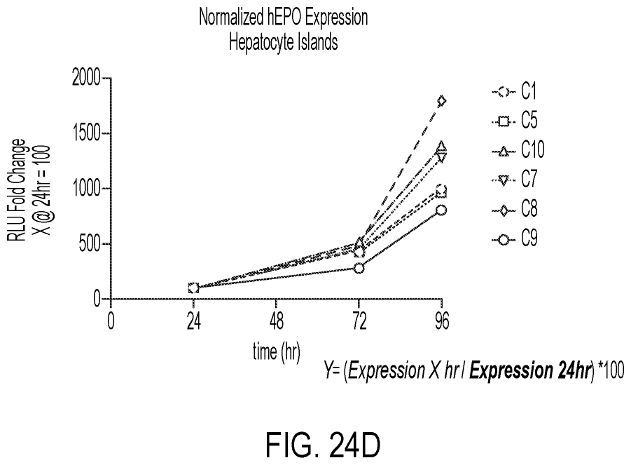

As shown in FIGS. 24A-24D, each test stop element was associated with comparable or increased protein levels in hepatocyte islands in vitro relative to the reference stop element.

### Example 26: Expression of a Target Protein Encoded by mRNA Having Different Stop Elements

This Example describes increased protein expression of a target protein encoded by an mRNA having different stop elements. The target proteins used in this example are firefly luciferase (ffLuc) and human erythropoietin (hEPO). The stop elements used in this example are C1 (reference), C5, C10, C7, C8 and C9. All stop elements were incorporated into an mRNA with the target protein open reading frame. The LNP formulated mRNAs were transfected into hepatocyte islands co-cultured with 3T3 fibroblasts or 3T3 fibroblasts alone. Expression of the encoded ffLuc was measured by a luciferase assay and the encoded EPO protein was measured by ELISA, at 24, 48, 72, and 96 hours post-transfection.

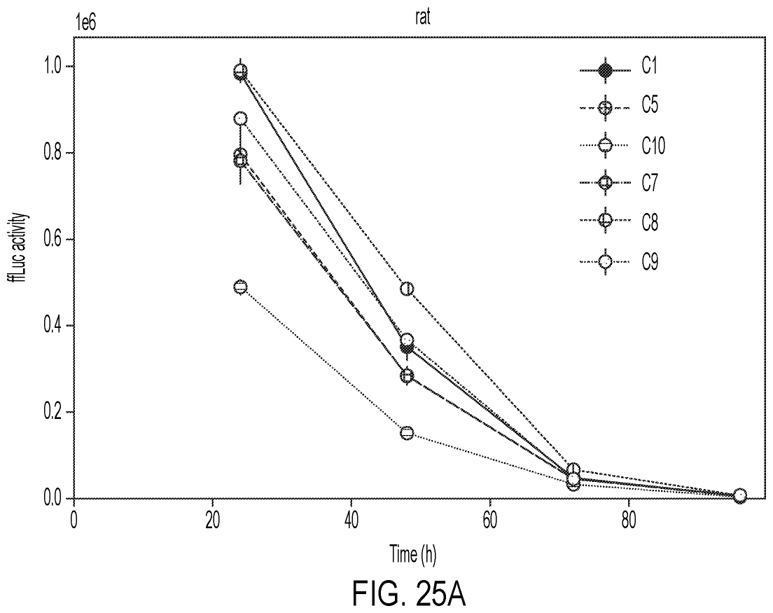

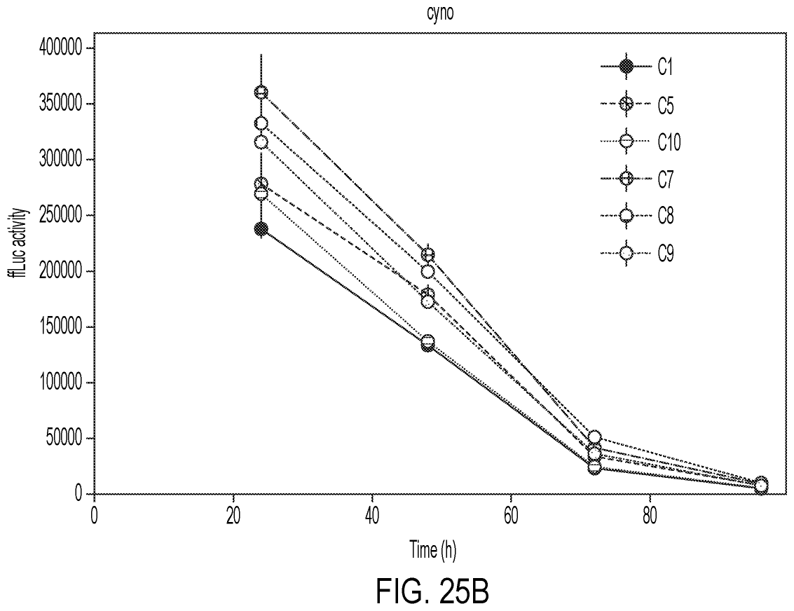

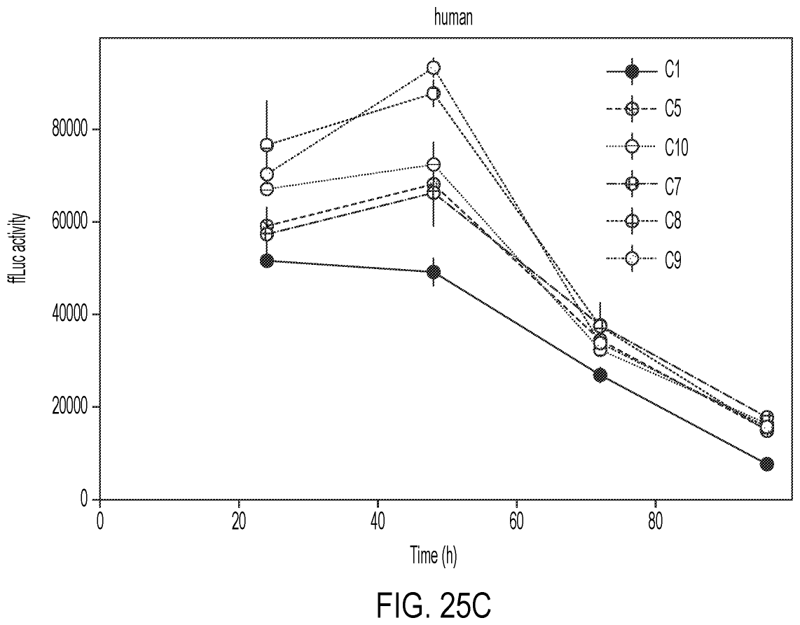

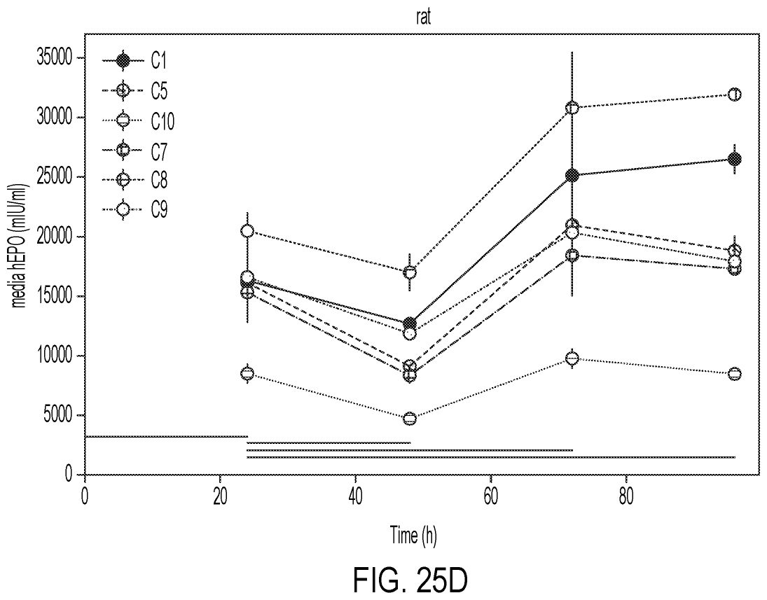

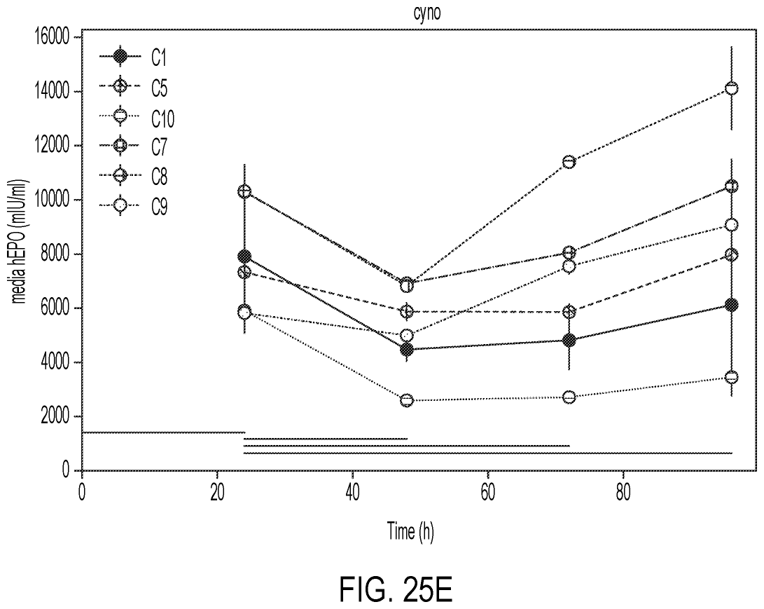

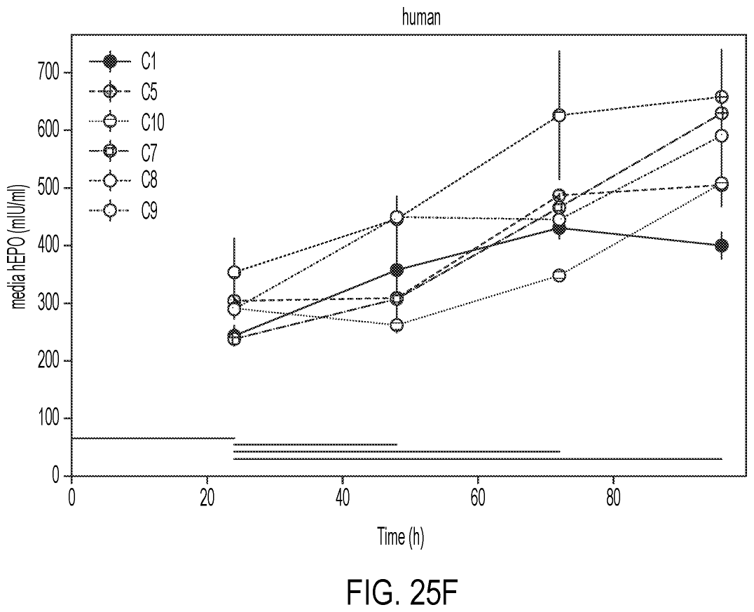

As shown in FIGS. 25A-25C, in cynomolgus and human HepatoPac each test stop element was associated with increased luciferase expression in vitro relative to the reference stop element. As shown in FIGS. 25D-25E, in cynomolgus and human HepatoPac most test stop elements were associated with increased hEPO protein expression in vitro relative to the reference stop element. In particular, C8 stop element was associated with longer half-life (ffLuc) and higher expression (hEPO) compared to the reference stop element.

### Example 27: In Vivo Effect of LNP Formulated mRNA Encoding an Immune Checkpoint Protein Having Different mRNA Elements

This Example describes the in vivo expression of an immune checkpoint protein encoded by an mRNA construct having a 5′ UTR, a stop element, and optionally a 3′ stabilizing region disclosed herein.

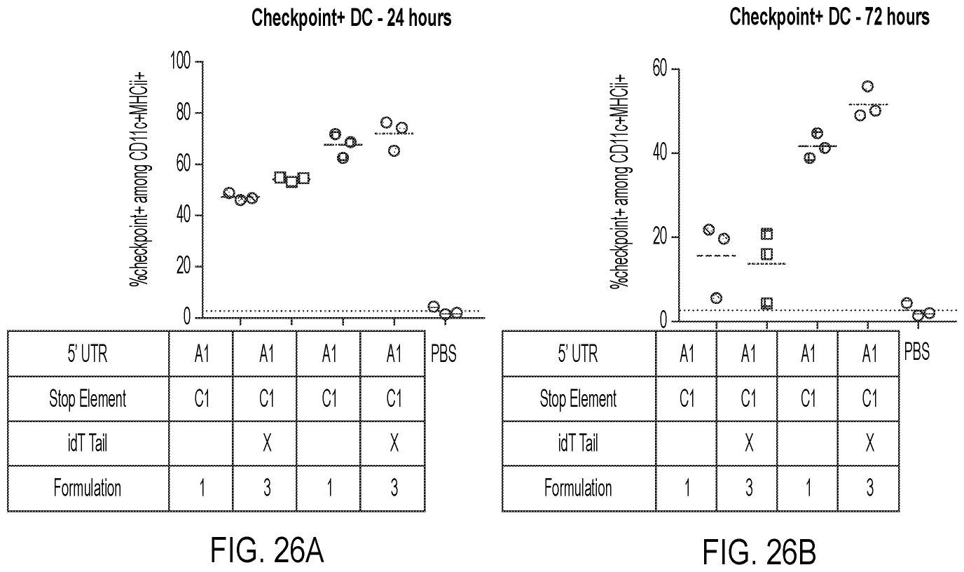

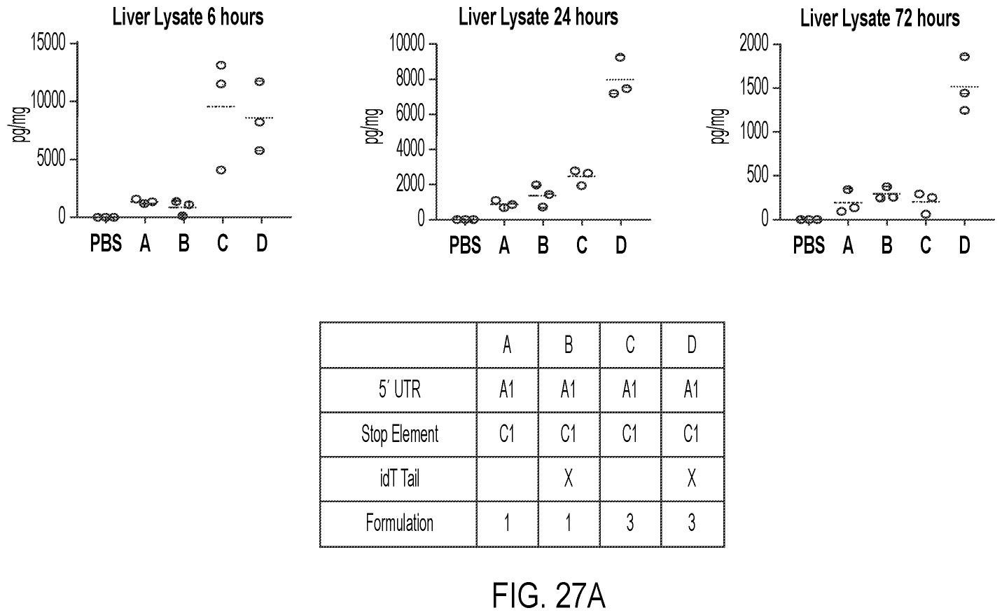

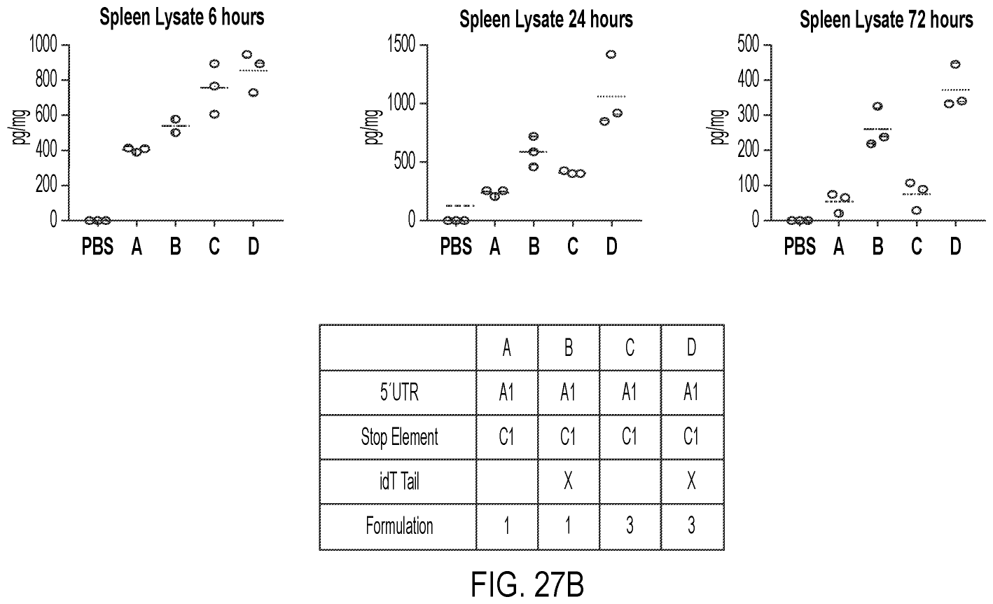

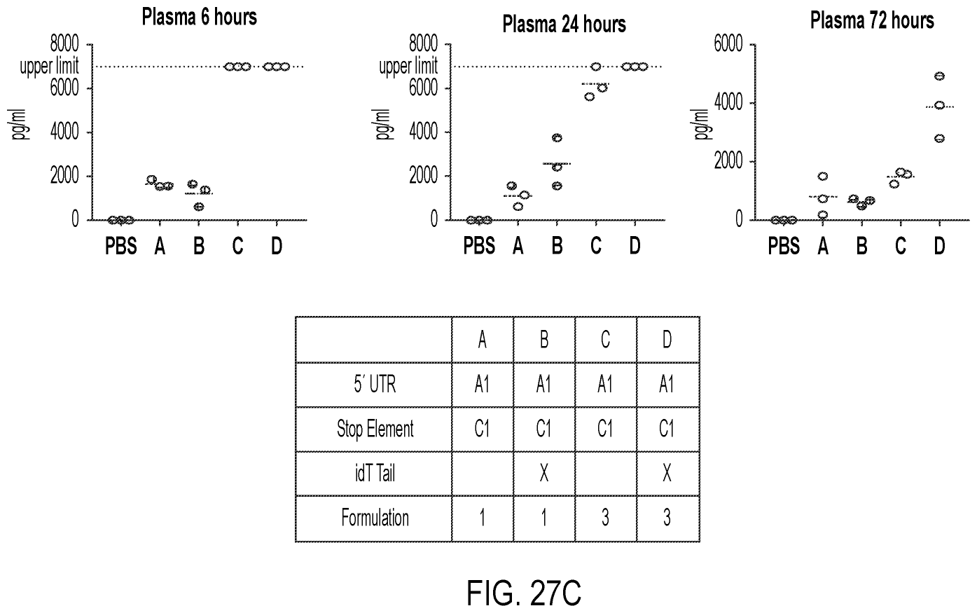

Briefly, mice were intravenously injected with 0.5 mg/kg of LNP formulated mRNAs encoding the immune checkpoint protein and having the mRNA elements specified in FIGS. 26A-27C. The 5′ UTR used in this example is A1. The stop element used in this example is C1. The stop element was incorporated into an mRNA with the target protein open reading frame. The 3′ stabilizing region used in this example includes an inverted thymidine (idT). Two LNP formulations (each comprising an ionizable amino lipid embraced by Formula IC, a phospholipid, cholesterol and a PEG lipid) were tested. 6, 24, and 72 hours post-dosing, mice were sacrificed, and dendritic cells were isolated for flow cytometry analysis and the spleen and livers were processed for evaluation of protein level by ELISA.

As shown in FIGS. 26A-26B, increased expression of the immune checkpoint protein among CD11c+MHCII+ cells (dendritic cells) was observed at 24 and 72 hours post-dosing in mice administered with LNP formulated mRNA having the 3′ stabilizing region (idT). Increased expression of the immune checkpoint protein was observed at 6, 24, and 72 hours post-dosing in the liver (FIG. 27A), spleen (FIG. 27B) and plasma (FIG. 27C) of mice dosed with LNP formulated mRNA having the 3′ stabilizing region (idT).

### Example 28: In Vivo Effect of LNP Formulated mRNA Encoding an Immune Checkpoint Protein Having Different mRNA Elements

This Example describes the in vivo expression of an immune checkpoint protein encoded by an mRNA construct having a 5′ UTR, a stop element, and optimally a 3′ stabilizing region disclosed herein.

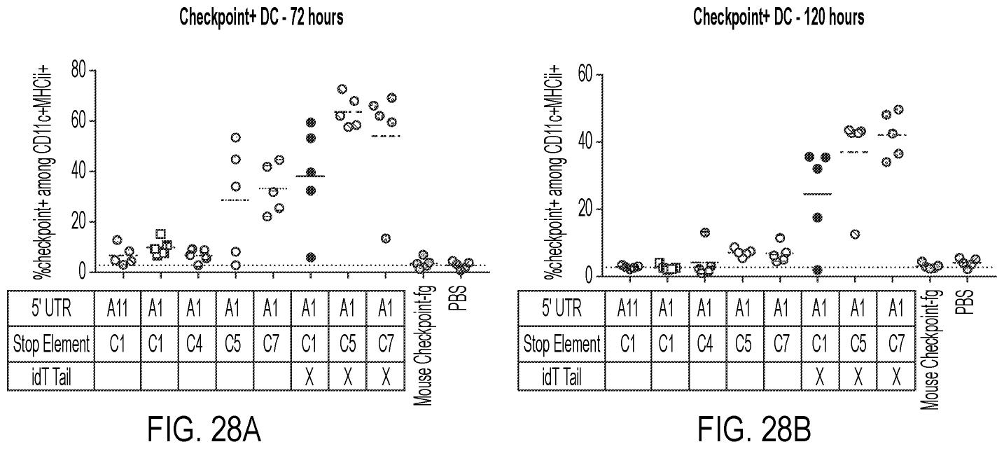

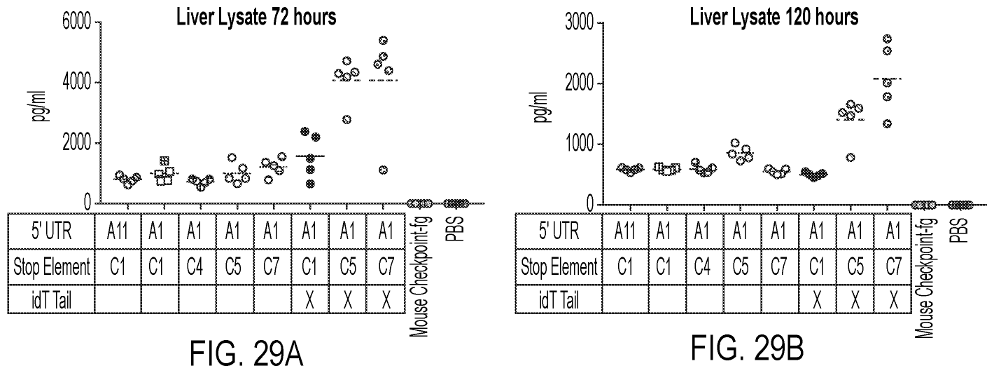

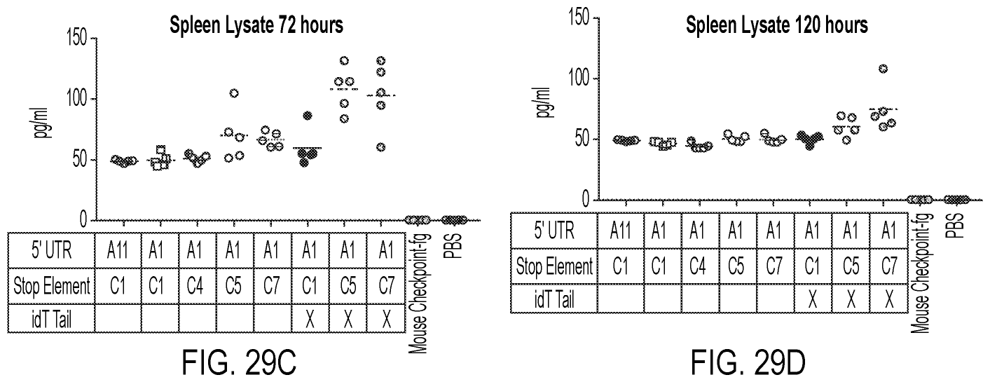

Briefly, mice were intravenously injected with 0.5 mg/kg of LNP formulated mRNAs encoding the immune checkpoint protein and having the mRNA elements specified in FIGS. 28A-29D. The 5′ UTR used in this example are A11 and A1. The stop elements used in this example are C1 (reference), C4, C5, and C7. All stop elements were incorporated into an mRNA with the immune checkpoint protein open reading frame. The 3′ stabilizing region used in this example includes an inverted thymidine (idT). 72 and 120 hours post-dosing, mice were sacrificed, and dendritic cells were isolated for flow cytometry analysis and the spleen and livers were processed for evaluation of protein level by ELISA.

As shown in FIGS. 28A-28B, C5 and C7 stop elements were associated with increased expression of the immune checkpoint protein at 72 and 120 hours post-dosing in mice. Also as shown in FIGS. 28A-28B, increased expression of the immune checkpoint protein was observed at 72 and 120 hours post-dosing in mice administered with LNP formulated mRNA having the stabilized tail.

Comparable or increased expression of the immune checkpoint protein was observed at 72 and 120 hours post-dosing in the liver (FIGS. 29A-29B) and spleen (FIGS. 29C-29D) of mice dosed with LNP formulated mRNA having C5 or C7 stop element relative to the reference stop element. Increased expression of the immune checkpoint protein was observed at 72 and 120 hours post-dosing in the liver (FIGS. 29A-29B) and spleen (FIGS. 29C-29D) of mice dosed with LNP formulated mRNA having the 3′ stabilizing region (idT).

### Example 29: Expression of an Immune Checkpoint Protein Encoded by mRNA Having Different Stop Elements

This Example describes increased protein expression of an immune checkpoint protein encoded by an mRNA having a stop element, and optionally a 3′ stabilizing region disclosed herein.

The stop elements used in this example are C1 (reference), C5 and C7. All stop elements were incorporated into an mRNA with the immune checkpoint protein open reading frame. The 3′ stabilizing region used in this example includes an inverted thymidine (idT). The LNP formulated mRNAs were transfected into hepatocyte islands co-cultured with 3T3 fibroblasts or 3T3 fibroblasts alone. Expression of the encoded immune checkpoint protein was measured by ELISA at 24, 48, 72, and 96 hours post-transfection.

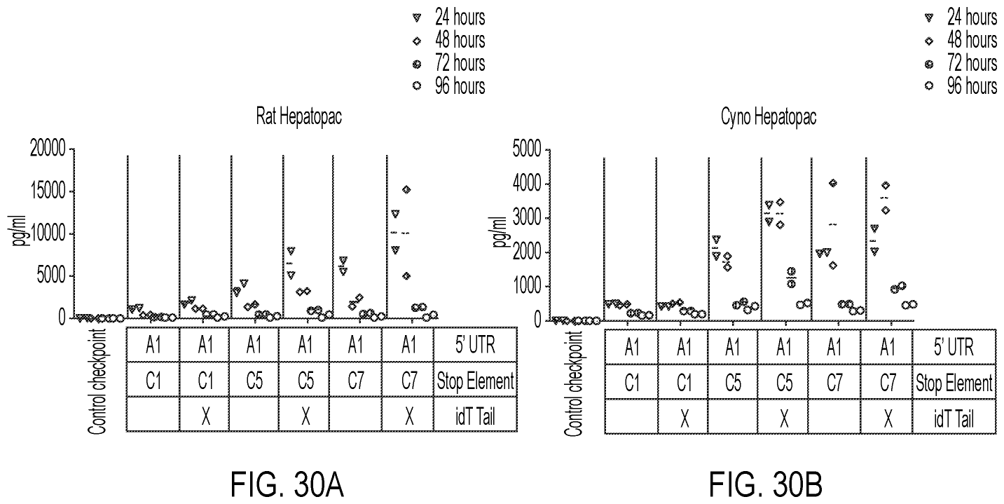

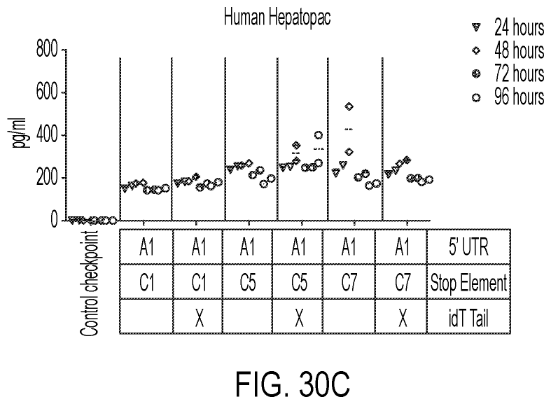

As shown in FIGS. 30A-30C, in rat, cynomolgus and human HepatoPac, C5 and C7 stop elements were associated with increased immune checkpoint protein expression in vitro relative to the reference stop element. Also as shown in FIGS. 30A-30C, mRNA having the 3′ stabilizing region showed a further increase in immune checkpoint protein expression.

### Example 30: Expression of an Immune Checkpoint Protein Encoded by mRNA Having Different Stop Elements

This Example describes the expression of an immune checkpoint protein in human peripheral blood mononuclear cells (hPBMC) encoded by an mRNA having a 5′ UTR, a stop element, and optionally a 3′ stabilizing region disclosed herein.

Freshly thawed hPBMCs from four donors were transfected with LNP formulated mRNAs encoding an immune checkpoint protein. The 5′ UTR used in this example are A11 and A1. The stop elements used in this example are C1 (reference), C5 and C7. All stop elements were incorporated into an mRNA with the immune checkpoint protein open reading frame. The 3′ stabilizing region used in this example includes an inverted thymidine (idT). Expression of the immune checkpoint protein was measured by flow cytometry at 24, 48, and 72 hours post-transfection.

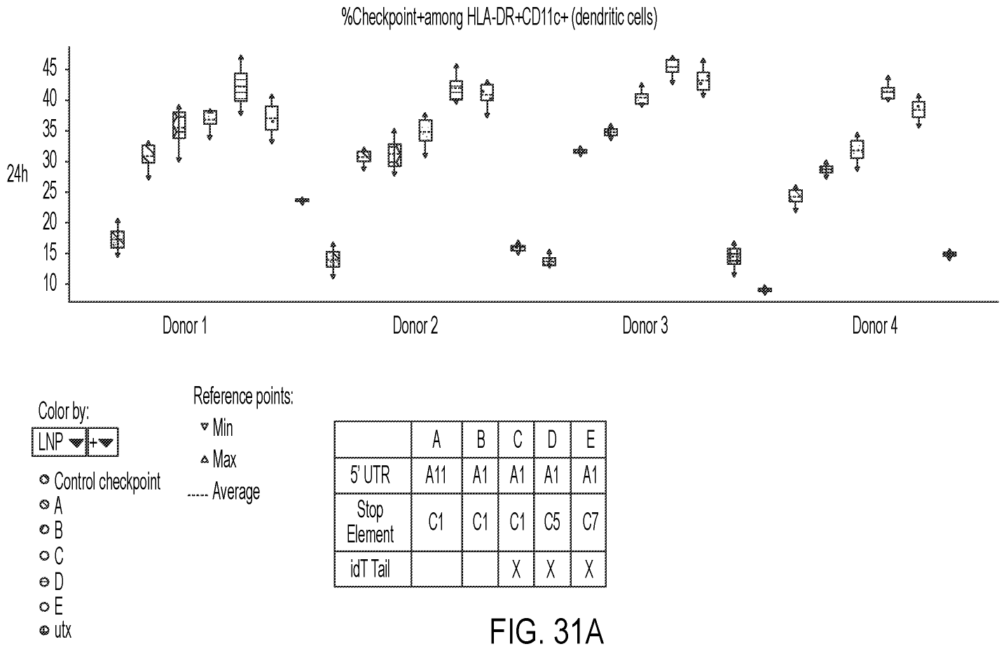

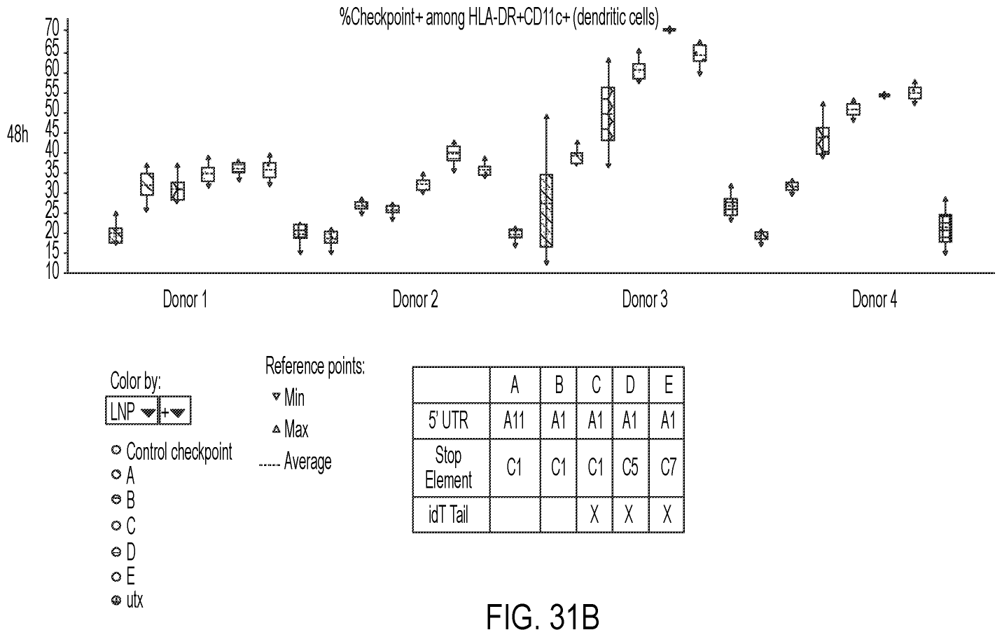

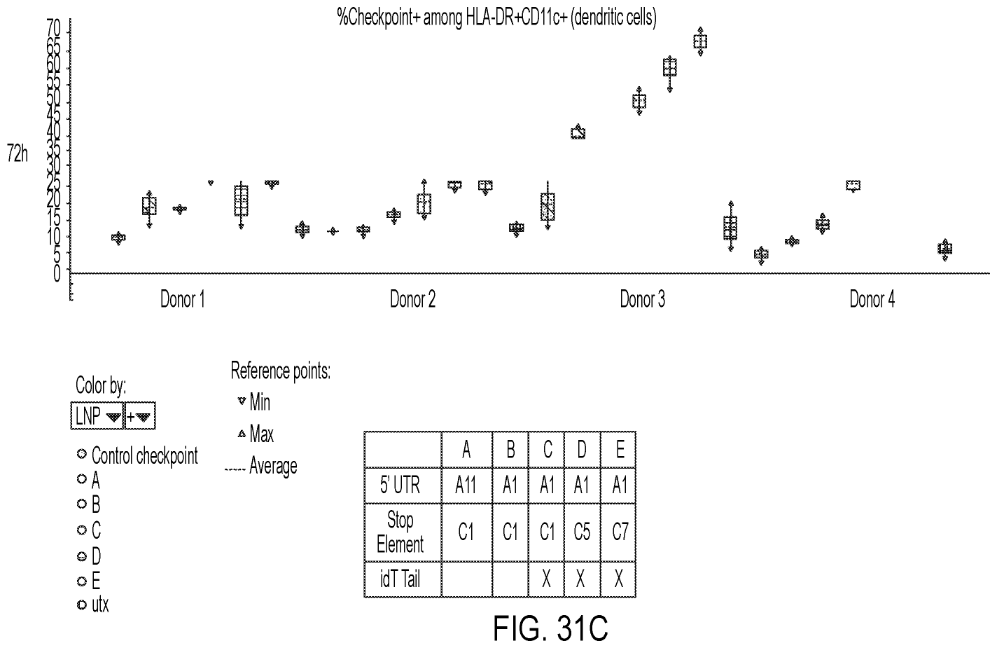

As shown in FIGS. 31A-31C, C5 and C7 stop elements were associated with increased immune checkpoint protein expression among HLA-DR+CD11c+ cells (dendritic cells) relative to the reference stop element. Also as shown in FIGS. 30A-30C, mRNA having the 3′ stabilizing region showed a further increase in immune checkpoint protein expression.

### Example 31: In Vivo Effect of LNP Formulated mRNA Encoding a Target Protein Having Different mRNA Elements

This Example describes the in vivo expression of a target protein encoded by an mRNA construct having a 5′ UTR and a stop element disclosed herein.

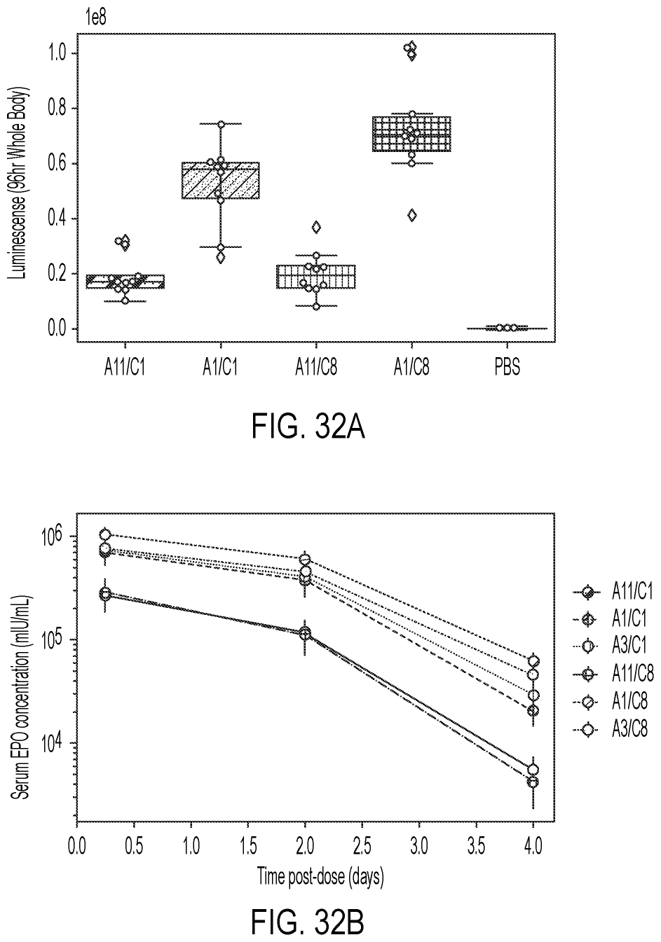

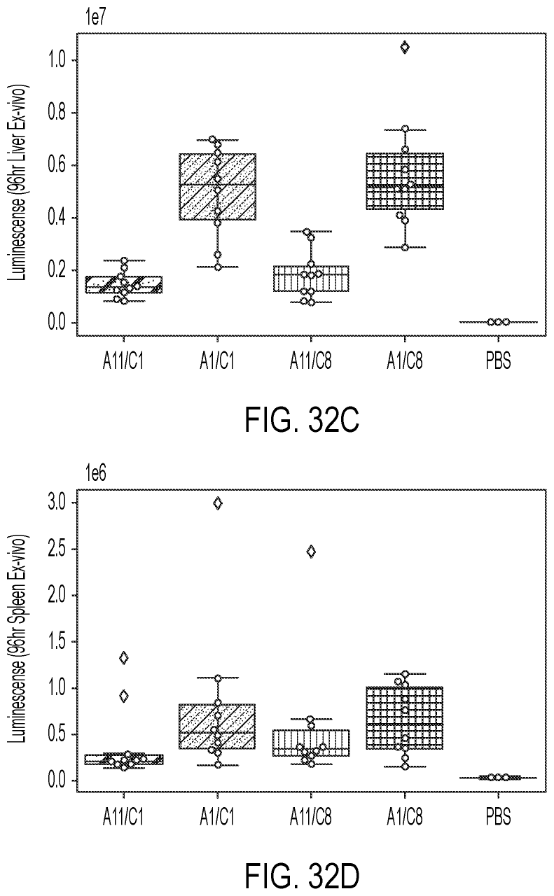

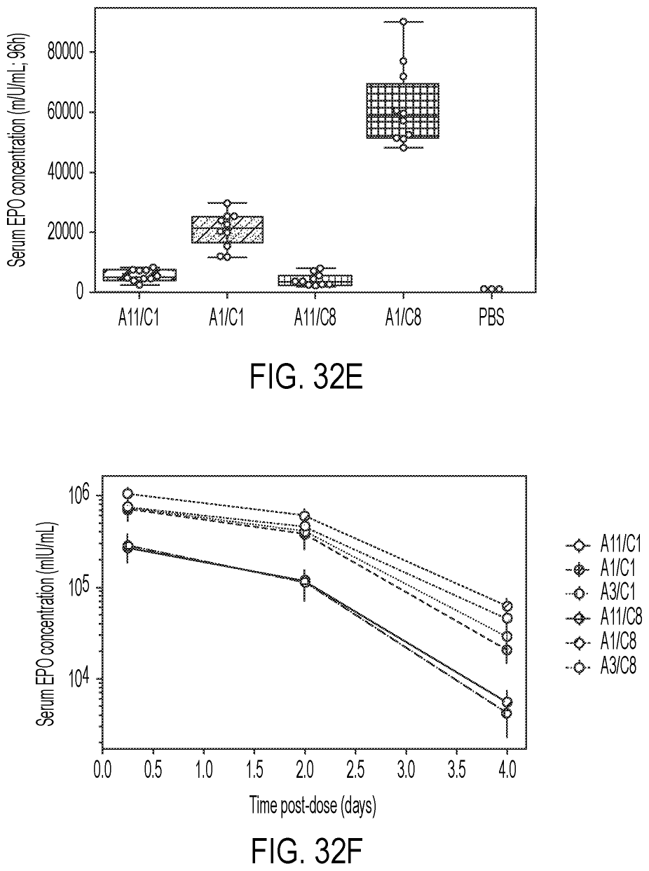

The target proteins used in this example are firefly luciferase (ffLuc) and human erythropoietin (hEPO). The 5′ UTRs used in this example are A11 (reference), A1, and A3. The stop elements used in the example are C1 (reference) and C8. All stop elements were incorporated into an mRNA with the target protein open reading frame. Briefly, mice were intravenously injected with 0.25 mg/kg of LNP formulated mRNAs encoding a target protein and having the mRNA elements specified in FIGS. 32A-32F. Expression of the encoded ffLuc protein was measured by whole-body luminescence imaging at 6 hours, 2 days, and 4 days post-dosing and ex vivo (spleen and liver) luminescence imaging at 4 days post-dosing. Expression of the encoded hEPO protein was measured by ELISA at 6 hours, 2 days and 4 days post-dosing.

As shown in FIGS. 32A-32B, the tested 5′ UTR/stop element combinations were associated with comparable or increased ffLuc protein levels in vivo at 6 hours, 2 days, and 4 days post-dosing relative to the reference 5′ UTR/stop element combination. Also as shown in FIGS. 32C-32D, the tested 5′ UTR/stop element combinations were associated with comparable or increased ffLuc protein levels ex vivo in liver and spleen at 4 days post-dosing relative to the reference 5′ UTR/stop element combination. As shown in FIGS. 32E-32F, the tested 5′ UTR/stop element combinations were associated with comparable or increased serum hEPO protein levels in vivo at 6 hours, 2 days, and 4 days post-dosing relative to the reference 5′ UTR/stop element combination.

### Example 32: Expression of a Target Protein Encoded by mRNA Having Different 3′ UTRs

This Example describes increased protein expression of a target protein encoded by an mRNA having a 3′ UTR disclosed herein.

The target proteins used in this example are two green fluoresce proteins. The 3′ UTRs used in this example are a 3′ UTR comprising B10 (“B10 3′ UTR”; reference) and a 3′ UTR comprising B18 (“B18 3′ UTR”). The 3′ UTRs were incorporated into an mRNA encoding the target protein and transfected into HeLa cells. Target protein levels were assessed in the course of 60 hours.

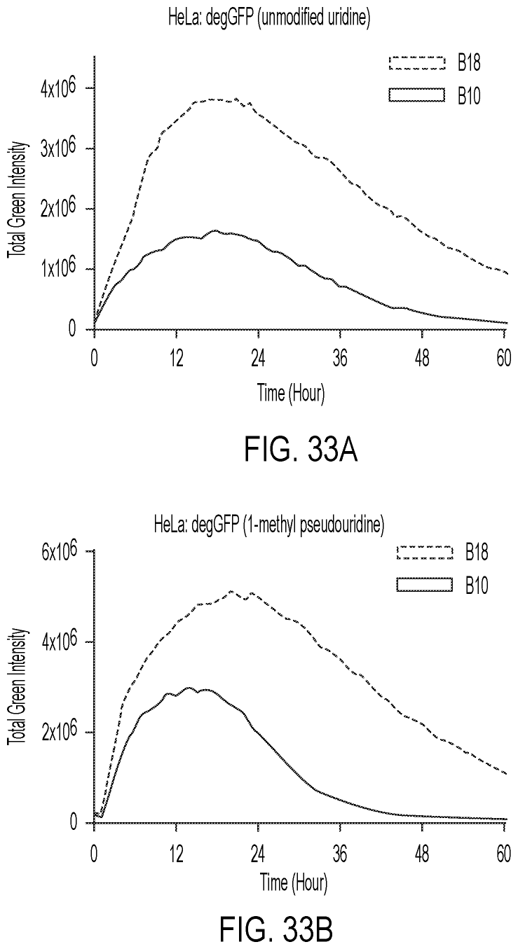

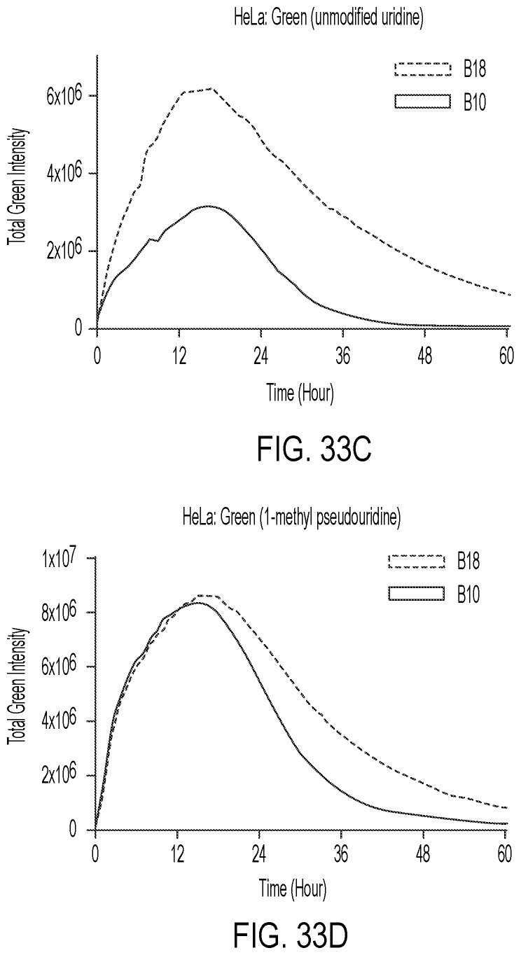

As shown in FIGS. 33A-33D, in HeLa cells the B18 3′ UTR was associated with increased protein levels in vitro relative to the reference 3′ UTR.

### Example 33: In Vivo Expression of a Target Protein Encoded by mRNA Having Different 3′ UTRs

This Example describes increased protein expression of a target protein encoded by an mRNA having a 3′ UTR disclosed herein.

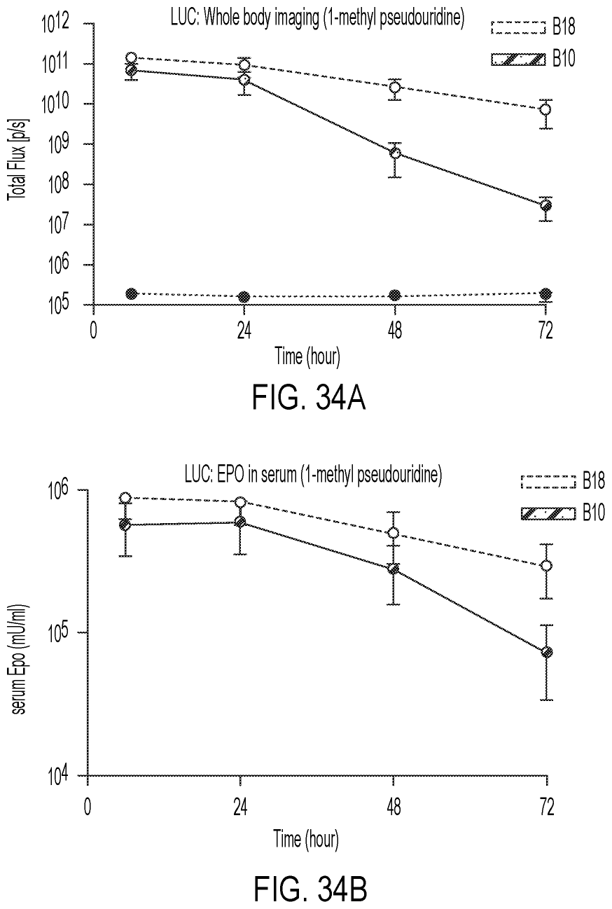

The target proteins used in this example are firefly luciferase (ffLuc) and human erythropoietin (hEPO). The 3′ UTRs used in this example are a 3′ UTR comprising B10 (“B10 3′ UTR”; reference) and a 3′ UTR comprising B18 (“B18 3′ UTR”). Mice were intravenously injected with 0.25 mg/kg of LNP formulated mRNAs having the 3′ UTRs specified in FIGS. 34A-34B. Expression of the encoded ffLuc protein was measured by whole-body luminescence imaging at 6 hours, 1, 2, and 4 days post-dosing. Expression of the encoded hEPO protein was measured by ELISA at 6 hours, 1, 2, and 4 days post-dosing.

As shown in FIG. 34A, the B18 3′ UTR was associated with increased ffLuc protein levels in vivo at 6 hours, 1, 2, and 4 days post-dosing relative to the reference 3′ UTR. Similarly, as shown in FIG. 34B, the B18 3′ UTR was associated with increased serum hEPO protein levels in vivo at 6 hours, 1, 2, and 4 days post-dosing relative to the reference 3′ UTR.

### Example 34: Expression of a Target Protein Encoded by mRNA Having Different 5′ UTR

This Example describes increased protein expression of a target protein encoded by an mRNA having different 5′ UTR elements.

The target protein used in this example is firefly luciferase (ffLuc). The 5′ UTRs used in this example are A12, A14, A15, A18, A20, A26, A27, and A11 (reference). The 5′ UTRs were incorporated into an mRNA encoding the target protein and transfected into HEK293 and HeLa cells. Target protein levels were assessed by luciferase assay at the 5, 24, 30, 48, 72, 96, and 120 hours post-administration.

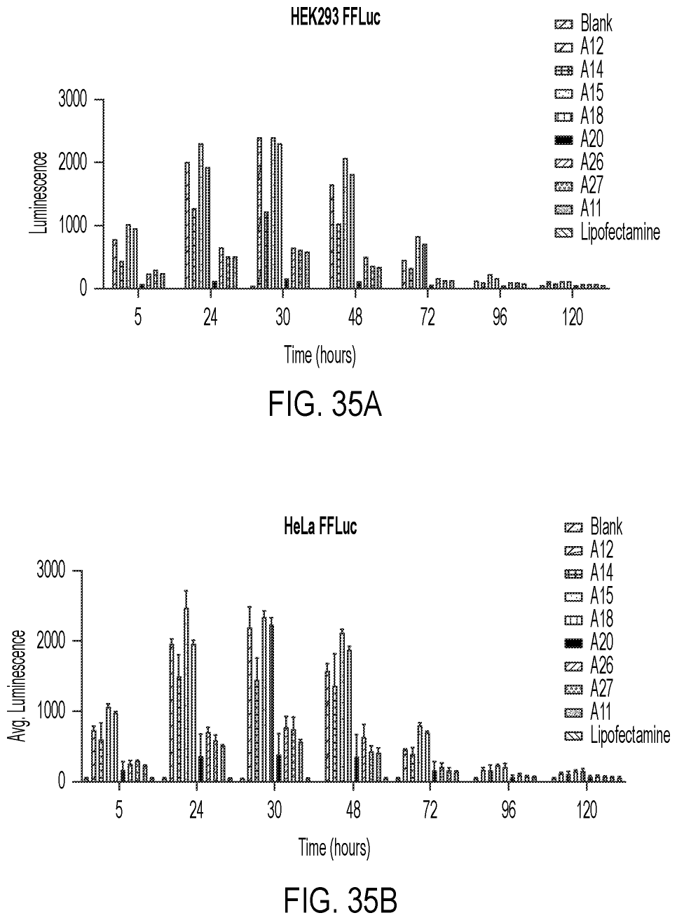

As shown in FIGS. 35A-35B, in HEK293 and HeLa cells at least A20, A26, A15, and A18 5′ UTRs were associated with increased ffLuc protein levels in vitro relative to the reference 5′ UTR.

### Example 35: In Vivo Expression of a Target Protein Encoded by mRNA Having Different 5′ UTRs

This Example describes increased protein expression of a target protein encoded by an mRNA having a 5′ UTR disclosed herein. The target protein used in this example is firefly luciferase (ffLuc). The 5′ UTRs used in this example are A12, A14, A20, A26, A27, A15, and A11 (reference).

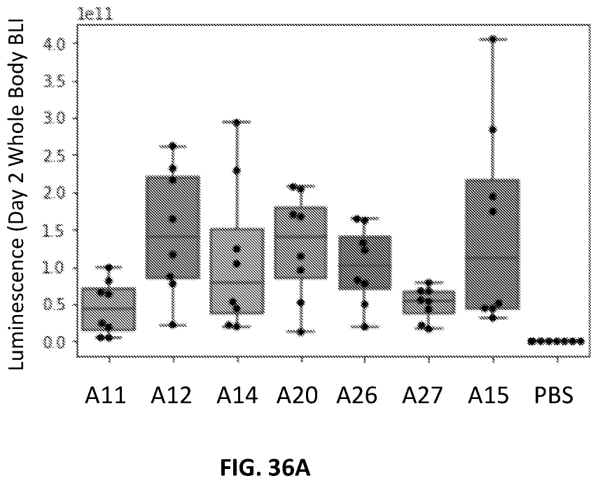

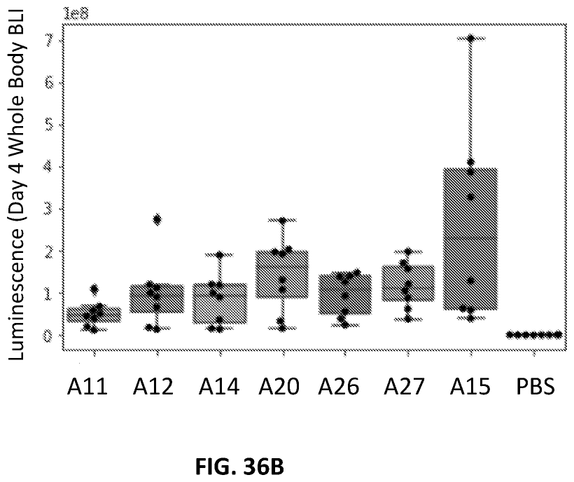

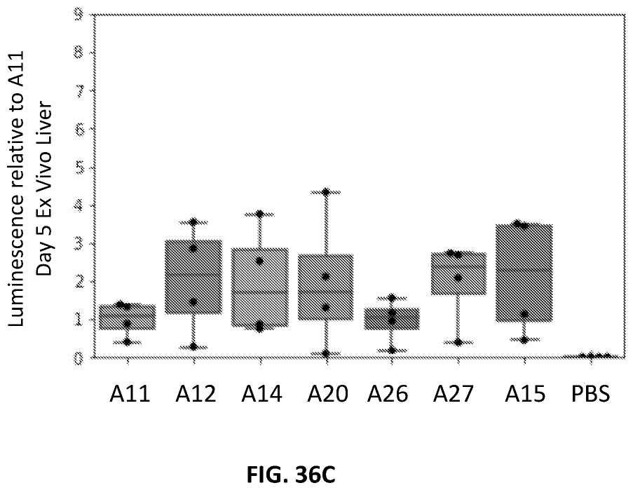

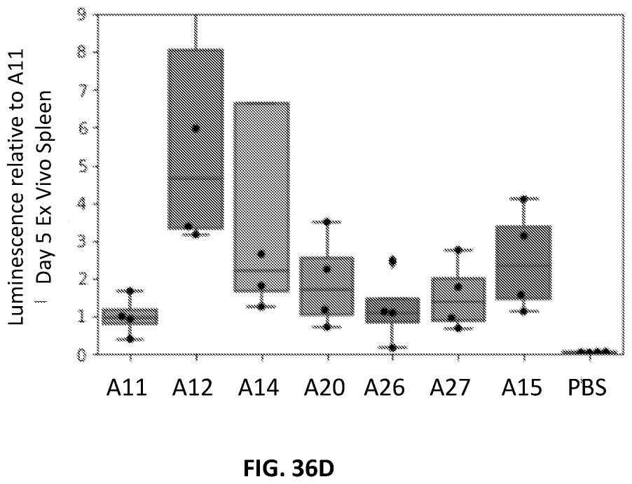

Mice were intravenously injected with 0.5 mg/kg of LNP formulated mRNAs having the 5′ UTRs specified in FIGS. 36A-36D. Expression of the encoded ffLuc protein was measured by whole-body luminescence imaging at 2 and 4 days post-dosing.

As shown in FIG. 36A, nearly all 5′ UTRs tested were associated with increased ffLuc protein levels in vivo at 2 days post-dosing relative to the reference 5′ UTR. As shown in FIG. 36B, all 5′ UTRs tested were associated with increased ffLuc protein levels in vivo at 4 days post-dosing relative to the reference 5′ UTR. At day 5, liver and spleens were harvested for ex-vivo luminescence imaging. The plot in FIG. 36C depicts the increase in expression in liver cells associated with various 5′ UTRs relative to the reference 5′ UTR. Similarly, the plot in FIG. 36D depicts the increase in expression in spleen cells associated with various 5′ UTRs relative to the reference 5′ UTR.

### Example 36: Production of LNP Compositions

### A. Production of Nanoparticle Compositions

In order to investigate safe and efficacious nanoparticle compositions for use in the delivery of therapeutic and/or prophylactics to cells, a range of formulations are prepared and tested. Specifically, the particular elements and ratios thereof in the lipid component of nanoparticle compositions are optimized.

Nanoparticles can be made with mixing processes such as microfluidics and T-junction mixing of two fluid streams, one of which contains the therapeutic and/or prophylactic and the other has the lipid components.

Lipid compositions are prepared by combining a lipid according to Formulae (I), (IA), (IB), (IC), (II), (IIa), (IIb), (IIc), (IId), (IIe), (IIf), (IIg), (III), (IIIa1), (IIIa2), (IIIa3), (IIIa4), (IIIa5), (IIIa6), (IIIa7), or (IIIa8) or a non-cationic helper lipid (such as DOPE, or DSPC obtainable from Avanti Polar Lipids, Alabaster, Ala.), a PEG lipid (such as 1,2 dimyristoyl sn glycerol methoxypolyethylene glycol, also known as PEG-DMG, obtainable from Avanti Polar Lipids, Alabaster, Ala.), and a phytosterol (optionally including a structural lipid such as cholesterol) at concentrations of about, e.g., 50 mM in a solvent, e.g., ethanol. Solutions should be refrigerated for storage at, for example, −20′ C. Lipids are combined to yield desired molar ratios (see, for example, Table 11 below) and diluted with water and ethanol to a final lipid concentration of e.g., between about 5.5 mM and about 25 mM. Phytosterol* in Table 11 refers to phytosterol or optionally a combination of phytosterol and structural lipid such as beta-phytosterol and cholesterol.

TABLE 11
Exemplary formulations of LNP compositions

| Composition | |
|---|---|
| (mol %) | Components |
| 40:20:38.5:1.5 | Ionizable lipid:Phospholipid:Phytosterol*:PEG-DMG |
| 45:15:38.5:1.5 | Ionizable lipid:Phospholipid:Phytosterol*:PEG-DMG |
| 50:10:38.5:1.5 | Ionizable lipid:Phospholipid:Phytosterol*:PEG-DMG |
| 55:5:38.5:1.5 | Ionizable lipid:Phospholipid:Phytosterol*:PEG-DMG |
| 60:5:33.5:1.5 | Ionizable lipid:Phospholipid:Phytosterol*:PEG-DMG |
| 45:20:33.5:1.5 | Ionizable lipid:Phospholipid:Phytosterol*:PEG-DMG |
| 50:20:28.5:1.5 | Ionizable lipid:Phospholipid:Phytosterol*:PEG-DMG |
| 55:20:23.5:1.5 | Ionizable lipid:Phospholipid:Phytosterol*:PEG-DMG |
| 60:20:18.5:1.5 | Ionizable lipidd:Phospholipid:Phytosterol*:PEG-DMG |
| 40:15:43.5:1.5 | Ionizable lipid:Phospholipid:Phytosterol*:PEG-DMG |
| 50:15:33.5:1.5 | Ionizable lipid:Phospholipid:Phytosterol*:PEG-DMG |
| 55:15:28.5:1.5 | Ionizable lipid:Phospholipid:Phytosterol*:PEG-DMG |
| 60:15:23.5:1.5 | Ionizable lipid:Phospholipid:Phytosterol*:PEG-DMG |
| 40:10:48.5:1.5 | Ionizable lipid:Phospholipid:Phytosterol*:PEG-DMG |
| 45:10:43.5:1.5 | Ionizable lipid:Phospholipid:Phytosterol*:PEG-DMG |
| 55:10:33.5:1.5 | Ionizable lipid:Phospholipid:Phytosterol*:PEG-DMG |
| 60:10:28.5:1.5 | Ionizable lipidd:Phospholipid:Phytosterol*:PEG-DMG |
| 40:5:53.5:1.5 | Ionizable lipid:Phospholipid:Phytosterol*:PEG-DMG |
| 45:5:48.5:1.5 | Ionizable lipid:Phospholipid:Phytosterol*:PEG-DMG |
| 50:5:43.5:1.5 | Ionizable lipid:Phospholipid:Phytosterol*:PEG-DMG |
| 40:20:40:0 | Ionizable lipid:Phospholipid:Phytosterol*:PEG-DMG |
| 45:20:35:0 | Ionizable lipid:Phospholipid:Phytosterol*:PEG-DMG |
| 50:20:30:0 | Ionizable lipidd:Phospholipid:Phytosterol*:PEG-DMG |
| 55:20:25:0 | Ionizable lipid:Phospholipid:Phytosterol*:PEG-DMG |
| 60:20:20:0 | Ionizable lipid:Phospholipid:Phytosterol*:PEG-DMG |
| 40:15:45:0 | Ionizable lipid:Phospholipid:Phytosterol*:PEG-DMG |
| 45:15:40:0 | Ionizable lipid:Phospholipid:Phytosterol*:PEG-DMG |
| 50:15:35:0 | Ionizable lipid:Phospholipid:Phytosterol*:PEG-DMG |
| 55:15:30:0 | Ionizable lipidPhospholipid:Phytosterol* PEG-DMG |
| 60:15:25:0 | Ionizable lipid:Phospholipid:Phytosterol*:PEG-DMG |
| 40:10:50:0 | Ionizable lipid:Phospholipid:Phytosterol*:PEG-DMG |
| 45:10:45:0 | Ionizable lipidPhospholipid:Phytosterol* PEG-DMG |
| 50:0:48.5:1.5 | Ionizable lipid:Phospholipid:Phytosterol*:PEG-DMG |
| 50:10:40:0 | Ionizable lipid:Phospholipid:Phytosterol*:PEG-DMG |
| 55:10:35:0 | Ionizable lipid:Phospholipid:Phytosterol*:PEG-DMG |
| 60:10:30:0 | Ionizable lipidPhospholipid:Phytosterol* PEG-DMG |

Nanoparticle compositions including a therapeutic and/or prophylactic and a lipid component are prepared by combining the lipid solution with a solution including the therapeutic and/or prophylactic at lipid component to therapeutic and/or prophylactic wt:wt ratios between about 5:1 and about 50:1. The lipid solution is rapidly injected using a NanoAssemblr microfluidic based system at flow rates between about 10 ml/min and about 18 ml/min into the therapeutic and/or prophylactic solution to produce a suspension with a water to ethanol ratio between about 1:1 and about 4:1.

For nanoparticle compositions including an RNA, solutions of the RNA at concentrations of 0.1 mg/ml in deionized water are diluted in a buffer, e.g., 50 mM sodium citrate buffer at a pH between 3 and 4 to form a stock solution.

Nanoparticle compositions can be processed by dialysis to remove ethanol and achieve buffer exchange. Formulations are dialyzed twice against phosphate buffered saline (PBS), pH 7.4, at volumes 200 times that of the primary product using Slide-A-Lyzer cassettes (Thermo Fisher Scientific Inc., Rockford, Ill.) with a molecular weight cutoff of 10 kDa. The first dialysis is carried out at room temperature for 3 hours. The formulations are then dialyzed overnight at 4° C. The resulting nanoparticle suspension is filtered through 0.2 m sterile filters (Sarstedt, Nümbrecht, Germany) into glass vials and sealed with crimp closures. Nanoparticle composition solutions of 0.01 mg/ml to 0.10 mg/ml are generally obtained.

The method described above induces nano-precipitation and particle formation. Alternative processes including, but not limited to, T-junction and direct injection, may be used to achieve the same nano-precipitation.

### B. Characterization of Nanoparticle Compositions

A Zetasizer Nano ZS (Malvern Instruments Ltd, Malvern, Worcestershire, UK) can be used to determine the particle size, the polydispersity index (PDI) and the zeta potential of the nanoparticle compositions in 1×PBS in determining particle size and 15 mM PBS in determining zeta potential.

Ultraviolet-visible spectroscopy can be used to determine the concentration of a therapeutic and/or prophylactic (e.g., RNA) in nanoparticle compositions. 100 μL of the diluted formulation in 1×PBS is added to 900 μL of a 4:1 (v/v) mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, Calif.). The concentration of therapeutic and/or prophylactic in the nanoparticle composition can be calculated based on the extinction coefficient of the therapeutic and/or prophylactic used in the composition and on the difference between the absorbance at a wavelength of, for example, 260 nm and the baseline value at a wavelength of, for example, 330 nm.

For nanoparticle compositions including an RNA, a QUANT-IT™ RIBOGREEN® RNA assay (Invitrogen Corporation Carlsbad, Calif.) can be used to evaluate the encapsulation of an RNA by the nanoparticle composition. The samples are diluted to a concentration of approximately 5 μg/mL in a TE buffer solution (10 mM Tris-HCl, 1 mM EDTA, pH 7.5). 50 μL of the diluted samples are transferred to a polystyrene 96 well plate and either 50 μL of TE buffer or 50 μL of a 2% Triton X-100 solution is added to the wells. The plate is incubated at a temperature of 37° C. for 15 minutes. The RIBOGREEN® reagent is diluted 1:100 in TE buffer, and 100 μL of this solution is added to each well. The fluorescence intensity can be measured using a fluorescence plate reader (Wallac Victor 1420 Multilablel Counter; Perkin Elmer, Waltham, Mass.) at an excitation wavelength of, for example, about 480 nm and an emission wavelength of, for example, about 520 nm. The fluorescence values of the reagent blank are subtracted from that of each of the samples and the percentage of free RNA is determined by dividing the fluorescence intensity of the intact sample (without addition of Triton X-100) by the fluorescence value of the disrupted sample (caused by the addition of Triton X-100).

### C. In Vivo Formulation Studies

In order to monitor how effectively various nanoparticle compositions deliver therapeutic and/or prophylactics to targeted cells, different nanoparticle compositions including a particular therapeutic and/or prophylactic (for example, a modified or naturally occurring RNA such as an mRNA) are prepared and administered to rodent populations. Mice are intravenously, intramuscularly, subcutaneously, intraarterially, or intratumorally administered a single dose including a nanoparticle composition with a lipid nanoparticle formulation. In some instances, mice may be made to inhale doses. Dose sizes may range from 0.001 mg/kg to 10 mg/kg, where 10 mg/kg describes a dose including 10 mg of a therapeutic and/or prophylactic in a nanoparticle composition for each 1 kg of body mass of the mouse. A control composition including PBS may also be employed.

Upon administration of nanoparticle compositions to mice, dose delivery profiles, dose responses, and toxicity of particular formulations and doses thereof can be measured by enzyme-linked immunosorbent assays (ELISA), bioluminescent imaging, or other methods. For nanoparticle compositions including mRNA, time courses of protein expression can also be evaluated. Samples collected from the rodents for evaluation may include blood, sera, and tissue (for example, muscle tissue from the site of an intramuscular injection and internal tissue); sample collection may involve sacrifice of the animals.

Nanoparticle compositions including mRNA are useful in the evaluation of the efficacy and usefulness of various formulations for the delivery of therapeutic and/or prophylactics. Higher levels of protein expression induced by administration of a composition including an mRNA will be indicative of higher mRNA translation and/or nanoparticle composition mRNA delivery efficiencies. As the non-RNA components are not thought to affect translational machineries themselves, a higher level of protein expression is likely indicative of a higher efficiency of delivery of the therapeutic and/or prophylactic by a given nanoparticle composition relative to other nanoparticle compositions or the absence thereof.

### Example 37: Synthesis of Exemplary Compounds of Formula (I)

### 3-Butylheptyl 8-bromooctanoate

### Step 1: Synthesis of ethyl 3-butylhept-2-enoate

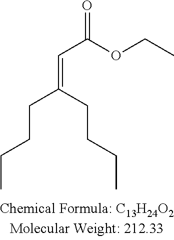

Triethyl phosphonoacetate (9.07 mL, 45.7 mmol) was added dropwise over 20 minutes to a suspension of sodium hydride (1.83 g, 45.7 mmol) in THE (14 mL) and the mixture was stirred at room temperature until gas evolution ceased (approximately 30 min). The reaction mixture was chilled to 0° C. and 5-nonanone (6.05 mL, 35.2 mmol) was added in portions. The reaction was gradually warmed to room temperature and allowed to stir under reflux for 24 h. The reaction was cooled to room temperature prior to being quenched with saturated aqueous sodium bicarbonate. The aqueous phase was extracted with diethyl ether, and the organic extracts were washed with brine, dried (MgSO₄), and concentrated to give a residue. The residue was purified by silica gel chromatography (0-20% EtOAc:hexanes) to afford ethyl 3-butylhept-2-enoate (5.27 g, 24.8 mmol, 71%) as an oil. ¹H NMR (300 MHz, CDCl₃) δ: ppm 5.62 (s, 1H); 4.14 (q, 2H, J=6.0 Hz); 2.59 (t, 2H, J=6.0 Hz); 2.14 (t, 2H, J=6.0 Hz); 1.50-1.23 (m, 11H); 0.99-0.82 (m, 6H).

### Step 2: Synthesis of ethyl 3-butylheptanoate

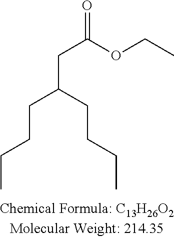

A steel Parr reactor equipped with a stir bar was charged with ethyl 3-butylhept-2-enoate (10.5 g, 49.5 mmol) in ethanol (50 mL). Palladium hydroxide on carbon (1.04 g, 7.42 mmol) was added and the vessel was sealed, evacuated, refilled with H₂ gas (3×), and the pressure was set to 200 psi. The reaction was stirred at 500 rpm, under 200 psi H₂ gas, at room temperature for 2 h. The vessel was then evacuated, refilled with N₂ gas, and opened. The crude reaction mixture was filtered through a Celite pad. The Celite pad was washed with EtOH and the crude material was concentrated to give ethyl 3-butylheptanoate (9.69 g, 45.2 mmol, 91%) as an oil. The compound was carried onto the next step without further purification. ¹H NMR (300 MHz, CDCl₃) δ: ppm 4.12 (q, 2H, J=9.0 Hz); 2.22 (d, 2H, J=6.0 Hz); 1.90-1.76 (m, 1H); 1.38-1.19 (m, 15H); 0.88 (br. t, 6H, J=6.0 Hz).

### Step 3: Synthesis of 3-butylheptan-1-ol

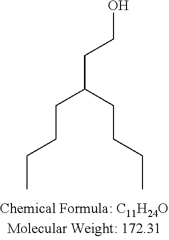

To a mixture of lithium aluminum hydride (850 mg, 22.4 mmol) in dry ether (23 mL) under N₂ at 0° C., was added dropwise ethyl 3-butylheptanoate (4.00 g, 18.7 mmol) in dry ether (15 mL). The mixture was stirred at room temperature for 2.5 h prior to being cooled to 0° C. Water (1 mL per g of LiAlH₄) was added to the solution dropwise, followed by the slow addition of 15% sodium hydroxide (1 mL per g of LiAlH₄) and water (3 mL per g of LiAlH₄). The solution was stirred for a few minutes at room temperature and filtered through a Celite pad. The Celite pad was washed with diethyl ether and the filtrate was concentrated. The crude material was purified by silica gel chromatography (0-40% EtOAc:hexanes) to afford 3-butylheptan-1-ol (3.19 g, 18.5 mmol, 99%) as an oil. ¹H NMR (300 MHz, CDCl₃) δ: ppm 3.66 (t, 2H, J=6.0 Hz); 1.53 (q, 2H, J=6.0 Hz); 1.46-1.36 (m, 1H); 1.35-1.21 (m, 12H); 1.18 (br. s, 1H); 0.89 (br. t, 6H, J=6.0 Hz).

### Step 4: Synthesis of 3-butylheptyl 8-bromooctanoate

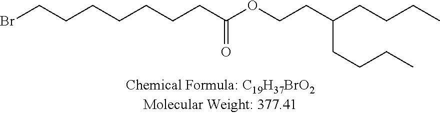 [SMILES: CCCCC(CCCC)CCOC(=O)CCCCCCCBr]

To a solution of 3-butylheptan-1-ol (3.19 g, 18.5 mmol), 8-bromooctanoic acid (4.96 g, 22.2 mmol), and DMAP (453 mg, 3.71 mmol) in methylene chloride (32 mL) at 0° C. was added EDCI (5.33 g, 27.8 mmol) and the reaction mixture stirred at room temperature overnight. The reaction mixture was then cooled to 0° C. and a solution of 10% hydrochloric acid (150 mL) was added slowly over 20 minutes. The layers were separated, and the organic layer was concentrated in vacuum to give a crude oil. The oil was dissolved in hexane (150 mL) and washed with a mixture of acetonitrile (150 mL) and 5% sodium bicarbonate (150 mL). The hexane layer was separated, dried (MgSO₄), and filtered. The solvent was removed under vacuum to give 3-butylheptyl 8-bromooctanoate (6.90 g, 18.3 mmol, 99%) as an oil. The compound was carried onto the next step without further purification. ¹H NMR (300 MHz, CDCl₃) δ: ppm 4.08 (t, 2H, J=6.0 Hz); 3.40 (t, 2H, J=6.0 Hz); 2.29 (t, 2H, J=6.0 Hz); 1.85 (pent., 2H, J=6.0 Hz); 1.69-1.52 (m, 4H); 1.49-1.20 (m, 19H); 0.89 (br. t, 6H, J=6.0 Hz).

### Heptadecan-9-yl 8-bromooctanoate

### Synthesis of heptadecan-9-yl 8-bromooctanoate

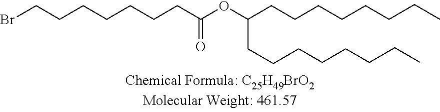 [SMILES: CCCCCCCCC(CCCCCCCC)OC(=O)CCCCCCCBr]

To a solution of heptadecan-9-ol, 8-bromooctanoic acid, and DMAP in methylene chloride was added EDC to afford heptadecan-9-yl 8-bromooctanoate.

### 3-Pentyloctyl 8-bromooctanoate

### Step 1: Synthesis of ethyl 3-pentyloct-2-enoate

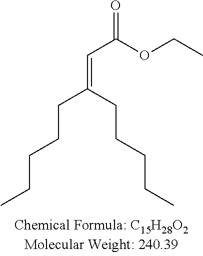 [SMILES: CCCCCC(=CC(=O)OCC)CCCCC]

Triethyl phosphonoacetate (10.6 mL, 53.4 mmol) was added dropwise over 20 minutes to a suspension of sodium hydride (2.13 g, 53.4 mmol) in THE (16 mL) and the mixture was stirred at room temperature until gas evolution ceased (approximately 30 min). The reaction mixture was chilled to 0° C. and 6-undecanone (8.42 mL, 41.1 mmol) was added in portions. The reaction was gradually warmed to room temperature and allowed to stir under reflux for 60 h. The reaction was cooled to room temperature prior to being quenched with saturated aqueous sodium bicarbonate. The aqueous phase was extracted with diethyl ether, and the organic extracts were washed with brine, dried (MgSO₄), and concentrated. The crude material was purified by silica gel chromatography (0-20% EtOAc:hexanes) to afford ethyl 3-pentyloct-2-enoate (8.76 g, 36.5 mmol, 89%) as an oil. ¹H NMR (300 MHz, CDCl₃) δ: ppm 5.61 (s, 1H); 4.14 (q, 2H, J=6.0 Hz); 2.58 (ddd, 2H, J=9.0, 9.0, 6.0 Hz); 2.13 (ddd, 2H, J=6.0, 6.0, 3.0 Hz); 1.52-1.38 (m, 3H); 1.38-1.23 (m, 12H); 0.93-0.86 (m, 6H).

### Step 2: Synthesis of ethyl 3-pentyloctanoate

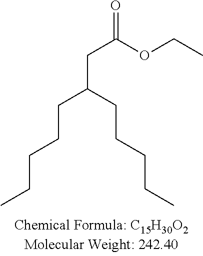 [SMILES: CCCCCC(CCCCC)CC(=O)OCC]

A steel Parr reactor equipped with a stir bar was charged with ethyl 3-pentyloct-2-enoate (8.76 g, 36.5 mmol) in ethanol (37 mL). Palladium hydroxide on carbon (768 mg, 5.47 mmol) was added and the vessel was sealed, evacuated, refilled with H₂ gas (3×), and the pressure was set to 200 psi. The reaction was stirred at 500 rpm, under 200 psi H₂ gas, at room temperature for 2 h. The vessel was then evacuated, refilled with N₂ gas, and opened. The crude reaction mixture was filtered through a Celite pad. The Celite pad was washed with EtOH and the crude material was concentrated to give ethyl 3-pentyloctanoate (8.45 g, 34.9 mmol, 96%) as an oil. The compound was carried onto the next step without further purification. ¹H NMR (300 MHz, CDCl₃) δ: ppm 4.12 (q, 2H, J=6.0 Hz); 2.22 (d, 2H, J=6.0 Hz); 1.92-1.77 (br. m, 1H); 1.37-1.19 (m, 19H); 0.88 (t, 6H, J=6.0 Hz).

### Step 3: Synthesis of 3-pentyloctan-1-ol

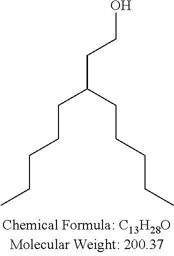

To a mixture of lithium aluminum hydride (1.59 g, 41.8 mmol) in dry ether (42 mL) under N₂ at 0° C., was added dropwise ethyl 3-pentyloctanoate (8.45 g, 34.9 mmol) in dry ether (28 mL). The mixture was stirred at room temperature for 2.5 h prior to being cooled to 0° C. Water (1 mL per g of LiAlH₄) was added to the solution dropwise, followed by the slow addition of 15% sodium hydroxide (1 mL per g of LiAlH₄) and water (3 mL per g of LiAlH₄). The solution was stirred for a few minutes at room temperature and filtered through a Celite pad. The Celite pad was washed with diethyl ether and the filtrate was concentrated. The crude material was purified by silica gel chromatography (0-40% EtOAc:hexanes) to afford 3-pentyloctan-1-ol (6.98 g, 34.9 mmol, 100%) as an oil. ¹H NMR (300 MHz, CDCl₃) δ: ppm 3.66 (t, 2H, J=6.0 Hz); 1.53 (q, 2H, J=6.0 Hz); 1.47-1.37 (br. s, 1H); 1.36-1.15 (m, 17H); 0.88 (t, 6H, J=6.0 Hz).

### Step 4: Synthesis of 3-Pentyloctyl 8-bromooctanoate

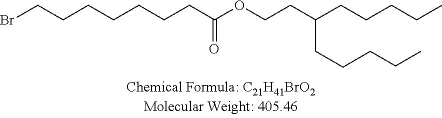 [SMILES: CCCCCC(CCCCC)CCOC(=O)CCCCCCCBr]

To a solution of 3-pentyloctan-1-ol (2.00 g, 9.98 mmol), 8-bromooctanoic acid (2.67 g, 12.0 mmol), and DMAP (244 mg, 2.00 mmol) in methylene chloride (18 mL) at 0° C. was added EDCI (2.87 g, 15.0 mmol) and the reaction mixture stirred at room temperature overnight. The reaction mixture was then cooled to 0° C. and a solution of 10% hydrochloric acid (70 mL) was added slowly over 20 minutes. The layers were separated, and the organic layer was concentrated in vacuum to give a crude oil. The oil was dissolved in hexane (70 mL) and washed with a mixture of acetonitrile (70 mL) and 5% sodium bicarbonate (70 mL). The hexane layer was separated, dried (MgSO₄), and filtered. The solvent was removed under vacuum to give 3-pentyloctyl 8-bromooctanoate (3.94 g, 9.72 mmol, 97%) as an oil. The compound was carried onto the next step without further purification. ¹H NMR (300 MHz, CDCl₃) δ: ppm 4.08 (t, 2H, J=6.0 Hz); 3.40 (t, 2H, J=6.0 Hz); 3.29 (t, 2H, J=6.0 Hz); 1.85 (pent., 2H, J=6.0 Hz); 1.68-1.52 (m, 4H); 1.49-1.19 (m, 23H); 0.88 (t, 6H, J=6.0 Hz).

### 3-Pentyloctyl 8-((3-((tert-butoxycarbonyl)amino)propyl)amino)octanoate

### Synthesis of 3-Pentyloctyl 8-((3-((tert-butoxycarbonyl)amino)propyl)amino)octanoate

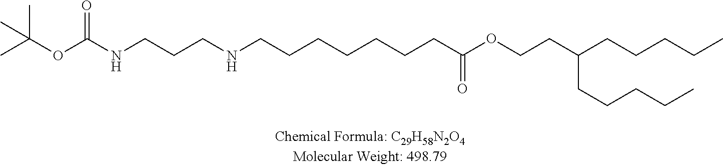 [SMILES: CCCCCC(CCCCC)CCOC(=O)CCCCCCCNCCCNC(=O)OC(C)(C)C]

To a solution of tert-butyl N-(3-aminopropyl)carbamate (15.5 g, 88.8 mmol) in EtOH (38 mL) was added 3-pentyloctyl 8-bromooctanoate (6.00 g, 14.8 mmol) in EtOH (36 mL) over the course of 20 min. The reaction was heated to 60° C. and allowed to stir at this temperature for 16 h. Upon cooling, the solvents were evaporated and the residue was diluted with ethyl acetate and washed with saturated aqueous NaHCO₃ and brine (5×) until no white precipitate was observed in the aqueous layer. The organic layer was separated, washed with brine, dried (MgSO₄), filtered, and concentrated. The residue was purified by flash chromatography (0-5-10-25-50-100% (mixture of 1% NH₄OH, 20% MeOH in dichloromethane) in dichloromethane) to give 3-pentyloctyl 8-((3-((tert-butoxycarbonyl)amino)propyl)amino)octanoate (4.23 g, 8.49 mmol, 57%) as an oil. ¹H NMR (300 MHz, CDCl₃) δ: ppm 5.17 (br. s, 1H); 4.07 (t, 2H, J=6.0 Hz); 3.19 (br. q, 2H, J=6.0 Hz); 2.66 (t, 2H, J=6.0 Hz); 2.56 (t, 2H, J=6.0 Hz); 2.28 (t, 2H, J=6.0 Hz); 1.70-1.52 (m, 6H); 1.51-1.39 (m, 3H); 1.44 (s, 9H); 1.36-1.19 (m, 22H); 0.88 (t, 6H, J=6.0 Hz).

### 3-Methoxy-4-(methylamino)cyclobut-3-ene-1,2-dione

### Synthesis of 3-methoxy-4-(methylamino)cyclobut-3-ene-1,2-dione

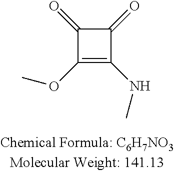

To a solution of 3,4-dimethoxy-3-cyclobutene-1,2-dione (1 g, 7 mmol) in 100 mL diethyl ether was added a 2M methylamine solution in THE (3.8 mL, 7.6 mmol) and a ppt. formed almost immediately. The mixture was stirred at room temperature for 24 hours, then filtered, the filter solids washed with diethyl ether and air-dried. The filter solids were dissolved in hot EtOAc, filtered, the filtrate allowed to cool to room temperature, then cooled to 0° C. to give a precipitate. This was isolated via filtration, washed with cold EtOAc, air-dried, then dried under vacuum to give 3-methoxy-4-(methylamino)cyclobut-3-ene-1,2-dione (0.70 g, 5 mmol, 73%) as a solid. ¹H NMR (300 MHz, DMSO-d₆) δ: ppm 8.50 (br. d, 1H, J=69 Hz); 4.27 (s, 3H); 3.02 (sdd, 3H, J=42 Hz, 4.5 Hz).

### 3-Butylheptyl 8-((8-(heptadecan-9-yloxy)-8-oxooctyl)(2-hydroxyethyl)amino)octanoate

### Step 1: Synthesis of heptadecan-9-yl 8-((2-hydroxyethyl)amino)octanoate

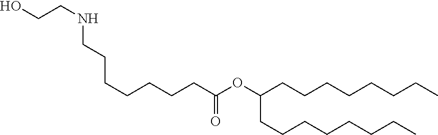 [SMILES: CCCCCCCCC(CCCCCCCC)OC(=O)CCCCCCCNCCO]

A solution of heptadecan-9-yl 8-bromooctanoate (10 g, 21.67 mmol) and ethanolamine (39.70 g, 649.96 mmol) in EtOH (5 mL) was heated to 65° C. for 16h. The reaction was cooled to rt and dissolved in ethyl acetate and extracted with water (4×). The organic layer was separated, washed with brine, dried with Na₂SO₄, filtered and evaporated under vacuum. The residue was purified by flash chromatography (ISCO) by 0-100% (a solution of 20% MeOH, 80% DCM, 1% NH₄₀H) in DCM to obtain heptadecan-9-yl 8-((2-hydroxyethyl)amino)octanoate (7.85 g, 82%). UPLC/ELSD: RT=2.06 min. MS (ES): m/z (MH⁺) 442.689 for C₂₇H₅₅NO₃. ¹H NMR (300 MHz, CDCl₃) δ: ppm 4.89 (p, 1H); 3.66 (t, 2H); 2.79 (t, 2H); 2.63 (m, 2H); 2.30 (t, 2H); 1.77-1.20 (m, 40H); 0.90 (m, 6H).

### Step 2: Compound 22: Synthesis of 3-Butylheptyl 8-((8-(heptadecan-9-yloxy)-8-oxooctyl)(2-hydroxyethyl)amino)octanoate

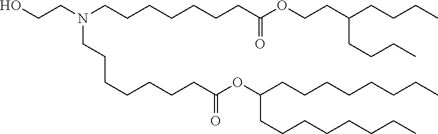 [SMILES: CCCCCCCCC(CCCCCCCC)OC(=O)CCCCCCCN(CCO)CCCCCCCC(=O)OCCC(CCCC)CCCC]

To a solution of 3-butylheptyl 8-bromooctanoate (6.15 g, 16.31 mmol) and heptadecan-9-yl 8-((2-hydroxyethyl)amino)octanoate (6.86 g, 15.53 mmol) in a mixture of CPME (15 mL) and acetonitrile (6 mL) was added potassium carbonate (8.59 g, 62.12 mmol) and potassium iodide (2.84 g, 17.08 mmol). The reaction was allowed to stir at 77° C. for 16 h. The reaction was cooled and filtered, and the volatiles were evaporated under vacuum. The residue was purified by flash chromatography (ISCO) by 0-100% (a solution of 20% MeOH, 80% DCM, 1% NH₄OH) in DCM to obtain 3-butylheptyl 8-((8-(heptadecan-9-yloxy)-8-oxooctyl)(2-hydroxyethyl)amino)octanoate (4.53 g, 37.8%). UPLC/ELSD: RT=3.04 min. MS (ES): m/z (MH⁺) 739.464 for C₄₆H₉₁NO₅. ¹H NMR (300 MHz, CDCl₃) δ: ppm 4.89 (p, 1H); 4.11 (m, 2H), 3.57 (bm, 2H); 2.73-2.39 (m, 6H); 2.30 (m, 4H); 1.72-1.17 (m, 64H); 0.92 (m, 12H).

### 3-Butylheptyl 8-((8-(heptadecan-9-yloxy)-8-oxooctyl)(3-((2-(methylamino)-3,4-dioxocyclobut-1-en-1-yl)amino)propyl)amino)octanoate

### Step 1: Synthesis of Heptadecan-9-yl 8-((3-((tert-butoxycarbonyl)amino)propyl)amino)octanoate

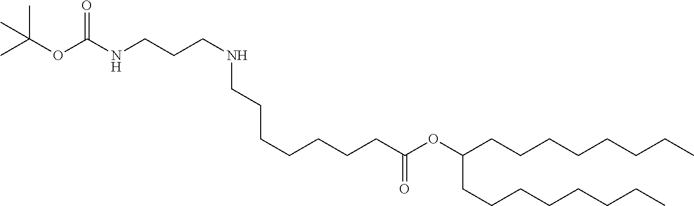 [SMILES: CCCCCCCCC(CCCCCCCC)OC(=O)CCCCCCCNCCCNC(=O)OC(C)(C)C]

A solution of tert-butyl N-(3-aminopropyl)carbamate (34.35 g, 197.15 mmol) in EtOH (200 mL) was heated to 65° C. and a solution of heptadecan-9-yl 8-bromooctanoate (26 g, 56.33 mmol) in EtOH (90 mL) was added over 3 h. The reaction was heated at 65° C. for 3h. The reaction was cooled to <50° C. and EtOH was evaporated under vacuum and azeotroped with heptane (4×). To a solution of crude product in 2-MeTHF (150 mL) 5% K₂CO₃ (150 mL) was added and the resulting mixture was stirred for 10 minutes. The two layers were allowed to form. The aqueous layer was removed and the 2-MeTHF layer was washed with 100 mL water (×3). The organic layer was separated, washed with brine, dried with Na₂SO₄, filtered and evaporated under vacuum. The residue was purified by flash chromatography (ISCO) by 0-100% (a solution of 20% MeOH, 80% DCM, 1% NH₄OH) in DCM to obtain heptadecan-9-yl 8-((3-((tert-butoxycarbonyl)amino)propyl)amino)octanoate (20 g, 63.9%). UPLC/ELSD: RT=2.34 min. MS (ES): m/z (MH⁺) 555.319 for C₃₃H₆₆N₂O₄. ¹H NMR (300 MHz, CDCl₃) δ: ppm 5.18 (bs, 1H); 4.89 (p, 1H); 3.22 (m, 2H); 2.64 (t, 2H); 2.59 (t, 2H); 2.30 (t, 2H); 1.73-1.21 (m, 50H); 0.90 (m, 6H).

### Step 2: Synthesis of 3-Butylheptyl 8-((3-((tert-butoxycarbonyl)amino)propyl)(8-(heptadecan-9-yloxy)-8-oxooctyl)amino)octanoate

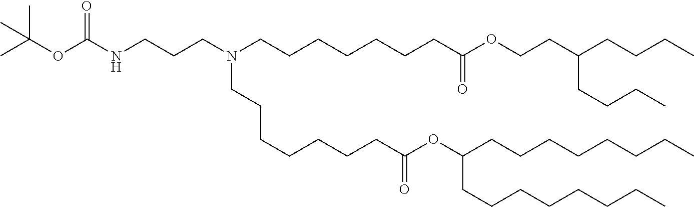 [SMILES: CCCCCCCCC(CCCCCCCC)OC(=O)CCCCCCCN(CCCCCCCC(=O)OCCC(CCCC)CCCC)CCCNC(=O)OC(C)(C)C]

To a solution of heptadecan-9-yl 8-((3-((tert-butoxycarbonyl)amino)propyl)amino)octanoate (11.76 g, 21.19 mmol) and 3-butylheptyl 8-bromooctanoate (9.2 g, 24.37 mmol) in propionitrile (52 mL) was added Potassium carbonate (4.39 g, 31.79 mmol) and Potassium iodide (0.53 g, 3.18 mmol). The reaction was heated at 80° C. for 16h. The reaction was cooled and filtered, and the volatiles were evaporated under vacuum. The residue was purified by flash chromatography (ISCO) by 0-100% (a solution of 20% MeOH, 80% DCM, 1% NH₄OH) in DCM to obtain 3-butylheptyl 8-((3-((tert-butoxycarbonyl)amino)propyl)(8-(heptadecan-9-yloxy)-8-oxooctyl)amino)octanoate (9.68 g, 53.6%). UPLC/ELSD: RT=3.07 min. MS (ES): m/z (MH⁺) 851.216 for C₅₂H₁₀₂N₂O₆. ¹H NMR (300 MHz, CDCl₃) δ: ppm 5.68 (bs, 1H); 4.90 (p, 1H); 4.11 (t, 2H); 3.20 (m, 2H); 2.52-2.24 (m, 10H); 1.76-1.20 (m, 74H); 0.90 (m, 12H).

### Step 3: Synthesis of 3-Butylheptyl 8-((3-aminopropyl)(8-(heptadecan-9-yloxy)-8-oxooctyl)amino)octanoate

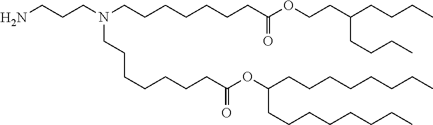 [SMILES: CCCCCCCCC(CCCCCCCC)OC(=O)CCCCCCCN(CCCN)CCCCCCCC(=O)OCCC(CCCC)CCCC]

To a solution of 3-butylheptyl 8-((3-((tert-butoxycarbonyl)amino)propyl)(8-(heptadecan-9-yloxy)-8-oxooctyl)amino)octanoate (7 g, 8.22 mmol) in DCM (25 mL) was added trifluoroacetic acid (9.4 mL, 123.32 mmol). The reaction was allowed to stir at rt for 2 h. The reaction was evaporated under vacuum. The residue was dissolved in mixture of methyl THF/heptane (1:9) and extracted with sat. sodium bicarbonate (3×). The organic layer was separated, washed with brine, dried with Na₂SO₄, filtered and evaporated under vacuum to obtain 3-Butylheptyl 8-((3-aminopropyl)(8-(heptadecan-9-yloxy)-8-oxooctyl)amino)octanoate. This was taken as a crude to the next step without further purification. UPLC/ELSD: RT=2.63 min. MS (ES): m/z (MHW) 751.305 for C₄₇H₉₄N₂O₄.

### Step 4: Compound 27. Synthesis of 3-Butylheptyl 8-((8-(heptadecan-9-yloxy)-8-oxooctyl)(3-((2-(methylamino)-3,4-dioxocyclobut-1-en-1-yl)amino)propyl)amino)octanoate

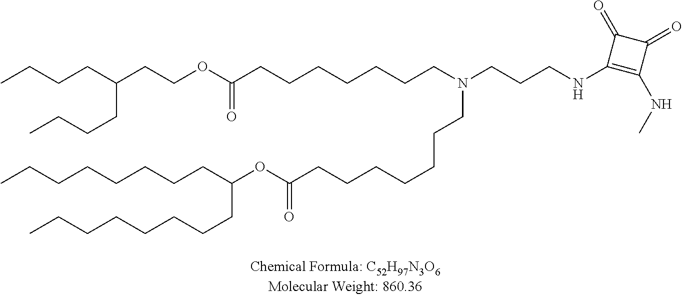 [SMILES: CCCCCCCCC(CCCCCCCC)OC(=O)CCCCCCCN(CCCCCCCC(=O)OCCC(CCCC)CCCC)CCCNc1c(NC)c(=O)c1=O]

To a solution of 3-butylheptyl 8-((3-aminopropyl)(8-(heptadecan-9-yloxy)-8-oxooctyl)amino)octanoate (7 g, 9.32 mmol) in methyl THF (31 mL) was added 3-methoxy-4-(methylamino)cyclobut-3-ene-1,2-dione (1.71 g, 12.11 mmol), and a aqueous solution of 10% Sodium bicarbonate (8.6 mL, 10.25 mmol). The reaction was allowed to stir at 50° C. for 2.5 h. The reaction was cooled to rt and diluted with heptane and extracted with water. The organic layer was separated, washed with brine, dried with Na₂SO₄, filtered and evaporated under vacuum. The residue was purified by flash chromatography (ISCO) by 0-100% (a solution of 20% MeOH, 80% DCM, 1% NH₄OH) in DCM to obtain 3-Butylheptyl 8-((8-(heptadecan-9-yloxy)-8-oxooctyl)(3-((2-(methylamino)-3,4-dioxocyclobut-1-en-1-yl)amino)propyl)amino)octanoate (5.4 g, 63%). UPLC/ELSD: RT=2.98 min. MS (ES): m/z (MH⁺) 861.714 for C₅₂H₉₇N₃O₆. ¹H NMR (300 MHz, CDCl₃) δ: ppm 4.89 (p, 1H); 4.10 (t, 2H); 3.75 (m, 2H); 3.39-3.20 (m, 5H); 3.08 (m, 4H); 2.31 (m, 4H); 2.12 (bm, 2H); 1.81-1.20 (m, 65H); 0.90 (m, 12H).

### Bis(3-pentyloctyl) 8,8′-((3-((2-(methylamino)-3,4-dioxocyclobut-1-en-1-yl)amino)propyl)azanediyl)dioctanoate

### Step 1: Synthesis of Bis(3-pentyloctyl) 8,8′-((3-((tert-butoxycarbonyl)amino)propyl) azanediyl)dioctanoate

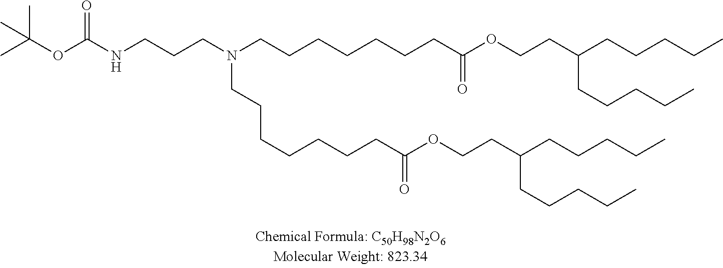 [SMILES: CCCCCC(CCCCC)CCOC(=O)CCCCCCCN(CCCCCCCC(=O)OCCC(CCCCC)CCCCC)CCCNC(=O)OC(C)(C)C]

To a solution of 3-pentyloctyl 8-bromooctanoate (5.61 g, 13.8 mmol) and 3-pentyloctyl 8-((3-((tert-butoxycarbonyl)amino)propyl)amino)octanoate (6.00 g, 12.0 mmol) in propionitrile (30 mL) was added potassium carbonate (2.49 g, 18.0 mmol) and iodopotassium (300 mg, 1.80 mmol). The reaction was allowed to stir at 80° C. for 16 h. Upon cooling to room temperature, the reaction mixture was filtered via vacuum filtration. The residue in the vessel and the filter cake on the funnel was washed twice with propionitrile. The filtrate was then concentrated in vacuo at 40° C. The crude residue was purified by silica gel chromatography (0-5-10-20-25-30-35-40-50-80-100% (mixture of 1% NH₄OH, 20% MeOH in dichloromethane) in dichloromethane) to give bis(3-pentyloctyl) 8,8′-((3-((tert-butoxycarbonyl)amino)propyl) azanediyl)dioctanoate (7.37 g, 8.95 mmol, 74%) as an oil. ¹H NMR (300 MHz, CDCl₃) δ: ppm 5.66 (br. s, 1H); 4.08 (t, 4H, J=6.0 Hz); 3.17 (br. q, 2H, J=6.0 Hz); 2.43 (t, 2H, J=6.0 Hz); 2.34 (br. t, 4H, J=6.0 Hz); 2.28 (t, 4H, J=9.0 Hz); 1.67-1.52 (m, 10H); 1.48-1.37 (m, 14H); 1.35-1.17 (m, 45H); 0.88 (t, 12H, J=6.0 Hz).

### Step 2: Synthesis of Bis(3-pentyloctyl) 8,8′-((3-aminopropyl)azanediyl)dioctanoate

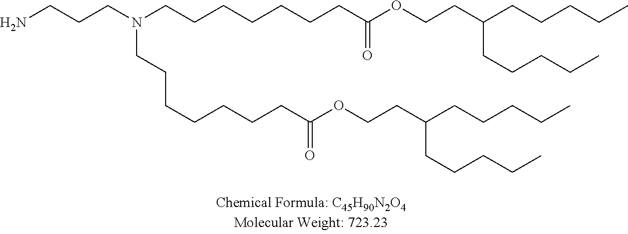 [SMILES: CCCCCC(CCCCC)CCOC(=O)CCCCCCCN(CCCN)CCCCCCCC(=O)OCCC(CCCCC)CCCCC]

To a round bottom flask equipped with a stir bar was added bis(3-pentyloctyl) 8,8′-((3-((tert-butoxycarbonyl)amino)propyl) azanediyl)dioctanoate (3.00 g, 3.64 mmol). The oil was dissolved in cyclopentyl methyl ether (8 mL) and stirred for 5 minutes. 3M HCl in cyclopentyl methyl ether (6.07 mL, 18.2 mmol) was added dropwise. After addition was complete, the reaction was heated to 40° C. for 1 hour and reaction completion was monitored by TLC/LCMS analysis. The reaction was cooled to room temperature, and then chilled to 0° C. 10% K₂CO₃ solution was then added dropwise to the reaction mixture. After addition was complete, the aqueous/cyclopentyl methyl ether emulsion was diluted with EtOAc and the resulting mixture stirred for 10 minutes. The solution was transferred to a separation funnel and the layers were separated. The organic layer was dried (MgSO₄), filtered, and concentrated. The residue was redissolved in heptane and washed twice with MeCN. The heptane layer was dried (MgSO₄), filtered, and concentrated to afford crude bis(3-pentyloctyl) 8,8′-((3-aminopropyl)azanediyl)dioctanoate (2.43 g, 3.36 mmol, 92%) as an oil. The crude material was carried onto the next step without further purification. ¹H NMR (300 MHz, CDCl₃) δ: ppm 4.08 (t, 4H, J=6.0 Hz); 2.98 (t, 2H, J=6.0 Hz); 2.71 (t, 2H, J=6.0 Hz); 2.54 (br. t, 4H, J=6.0 Hz); 2.28 (t, 6H, J=6.0 Hz); 1.76 (br. pentet, 2H, J=2.0 Hz); 1.66-1.52 (m, 9H); 1.52-1.43 (m, 4H); 1.37-1.18 (m, 45H); 0.88 (t, 12H, J=6.0 Hz).

### Step 3: Compound 30: Synthesis of Bis(3-pentyloctyl) 8,8′-((3-((2-(methylamino)-3,4-dioxocyclobut-1-en-1-yl)amino)propyl)azanediyl)dioctanoate

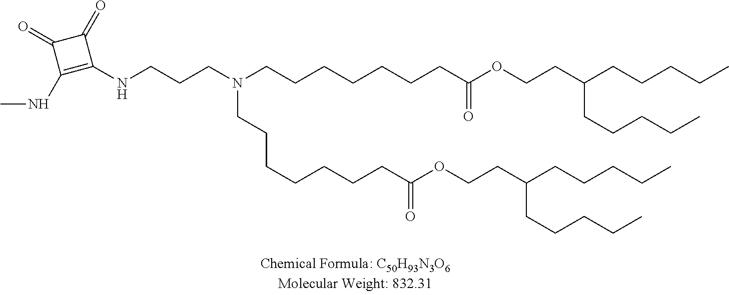 [SMILES: CCCCCC(CCCCC)CCOC(=O)CCCCCCCN(CCCCCCCC(=O)OCCC(CCCCC)CCCCC)CCCNc1c(NC)c(=O)c1=O]

To a round bottom flask equipped with a stir bar was added bis(3-pentyloctyl) 8,8′-((3-aminopropyl)azanediyl)dioctanoate (2.43 g, 3.36 mmol), 3-methoxy-4-(methylamino)cyclobut-3-ene-1,2-dione (616 mg, 4.36 mmol) and 2-Methyl THE (10 mL). 10% K₂CO₃ solution (10 mL) was added and the resulting biphasic mixture was heated to 45° C. and stirred vigorously for 3 hours. Reaction completion was monitored by TLC/LCMS analysis. Upon completion the mixture was allowed to cool to room temperature. The reaction was diluted with water, layers were separated, and the aqueous layer was extracted twice with heptane. The organics were combined, washed with water (3×), brine, and with a 1:1 acetonitrile/water mixture. The combined organics were then dried (Na₂SO₄), filtered, and concentrated. The crude residue was azeotroped and concentrated with DCM and MeOH three times to yield a pale yellow crude waxy oil. The crude residue was purified by silica gel chromatography (0-100% (mixture of 1% NH₄OH, 20% MeOH in dichloromethane) in dichloromethane) to give bis(3-pentyloctyl) 8,8′-((3-((2-(methylamino)-3,4-dioxocyclobut-1-en-1-yl)amino)propyl)azanediyl)dioctanoate (2.11 g, 2.54 mmol, 76%) as a solid. UPLC/ELSD: RT=2.79 min. MS (ES): m/z (MH⁺) 832.34 for C₅₀H₉₃N₃O₆. ¹H NMR (300 MHz, CDCl₃) δ: ppm 7.83 (br. s, 1H); 7.61 (br. s, 1H); 4.03 (t, 4H, J=9.0 Hz); 3.64 (br. s, 2H); 3.28 (br. d, 3H, J=6.0 Hz); 2.46 (t, 2H, J=9.0 Hz); 2.33 (br. t, 4H, J=6.0 Hz); 2.33 (t, 4H, J=9.0 Hz); 1.74 (br. pentet, 2H, J=6.0 Hz); 1.62-1.47 (m, 8H); 1.41-1.12 (m, 50H); 0.83 (t, 12H, J=9.0 Hz).

### 3-Butylheptyl 8-((3-((2-(methylamino)-3,4-dioxocyclobut-1-en-1-yl)amino)propyl)(8-oxo-8-((3-pentyloctyl)oxy)octyl)amino)octanoate

### Step 1: Synthesis of 3-butylheptyl 8-((3-((tert-butoxycarbonyl)amino)propyl)(8-oxo-8-((3-pentyloctyl)oxy)octyl)amino)octanoate

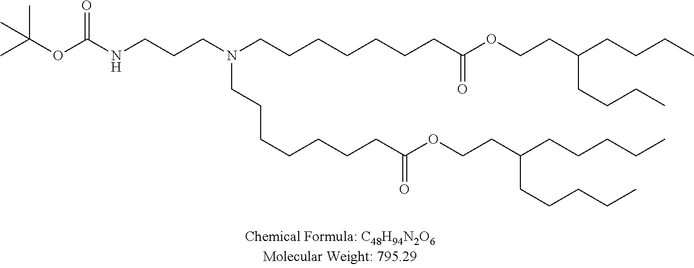 [SMILES: CCCCCC(CCCCC)CCOC(=O)CCCCCCCN(CCCCCCCC(=O)OCCC(CCCC)CCCC)CCCNC(=O)OC(C)(C)C]

To a solution of 3-butylheptyl 8-bromooctanoate (794 mg, 2.11 mmol) and 3-pentyloctyl 8-((3-((tert-butoxycarbonyl)amino)propyl)amino)octanoate (1.00 g, 2.01 mmol) in cyclopentyl methyl ether (9 mL) and actonitrile (9 mL) was added potassium carbonate (1.66 g, 12.0 mmol) and iodopotassium (366 mg, 2.21 mmol). The reaction was allowed to stir at 80° C. for 16 h. Upon cooling, the volatiles were evaporated under vacuum. The residue was diluted with dichloromethane and washed with water. The organic layer was separated, washed with brine, dried (MgSO₄), filtered, and concentrated. The crude residue was purified by silica gel chromatography (0-5-10-25-50-100% (mixture of 1% NH₄OH, 20% MeOH in dichloromethane) in dichloromethane) to give 3-butylheptyl 8-((3-((tert-butoxycarbonyl)amino)propyl)(8-oxo-8-((3-pentyloctyl)oxy)octyl)amino)octanoate (896 mg, 1.13 mmol, 56%) as an oil. UPLC/ELSD: RT=2.95 min. MS (ES): m/z (MH⁺) 795.59 for C₄₈H₉₄N₂O₆.

### Step 2: Synthesis of 3-butylheptyl 8-((3-aminopropyl)(8-oxo-8-((3-pentyloctyl)oxy)octyl)amino)octanoate

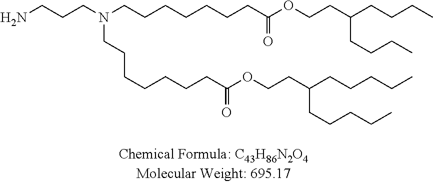 [SMILES: CCCCCC(CCCCC)CCOC(=O)CCCCCCCN(CCCN)CCCCCCCC(=O)OCCC(CCCC)CCCC]

To a solution of 3-butylheptyl 8-((3-((tert-butoxycarbonyl)amino)propyl)(8-oxo-8-((3-pentyloctyl)oxy)octyl)amino)octanoate (896 mg, 1.13 mmol) in methylene chloride (23 mL) was added trifluoroacetic acid (1.72 mL, 22.5 mmol). The reaction was allowed to stir at room temperature for 4 h. The reaction was quenched with saturated aqueous NaHCO₃ and extracted with dichloromethane. The organic layer was separated, washed with brine, dried (MgSO₄), filtered and concentrated. The crude material was purified by silica gel chromatography (0-5-10-25-50-100% (mixture of 1% NH₄OH, 20% MeOH in dichloromethane) in dichloromethane) to give 3-butylheptyl 8-((3-aminopropyl)(8-oxo-8-((3-pentyloctyl)oxy)octyl)amino)octanoate (632 mg, 0.91 mmol, 81%) as an oil. UPLC/ELSD: RT=2.47 min. MS (ES): m/z (MH⁺) 695.68 for C₄₃H₈₆N₂O₄.

### Step 3: Compound 54: Synthesis of 3-butylheptyl 8-((3-((2-(methylamino)-3,4-dioxocyclobut-1-en-1-yl)amino)propyl)(8-oxo-8-((3-pentyloctyl)oxy)octyl)amino)octanoate

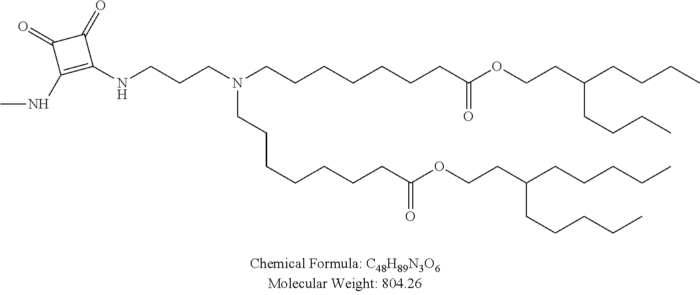 [SMILES: CCCCCC(CCCCC)CCOC(=O)CCCCCCCN(CCCCCCCC(=O)OCCC(CCCC)CCCC)CCCNc1c(NC)c(=O)c1=O]

To a solution of 3-butylheptyl 8-((3-aminopropyl)(8-oxo-8-((3-pentyloctyl)oxy)octyl)amino)octanoate (632 mg, 0.91 mmol) in ethanol (8 mL) was added 3-methoxy-4-(methylamino)cyclobut-3-ene-1,2-dione (192 mg, 1.36 mmol). The reaction was allowed to stir at 67° C. for 20 h. After 20 h, the reaction was cooled to room temperature and diluted with diethyl ether. The organics were washed with brine, dried (MgSO₄), filtered, and concentrated. The crude residue was purified by silica gel chromatography (0-5-10-25-50-100% (mixture of 1% NH₄OH, 20% MeOH in dichloromethane) in dichloromethane) to give 3-butylheptyl 8-((3-((2-(methylamino)-3,4-dioxocyclobut-1-en-1-yl)amino)propyl)(8-oxo-8-((3-pentyloctyl)oxy)octyl)amino)octanoate (240 mg, 0.30 mmol, 33%) as a solid. UPLC/ELSD: RT=2.67 min. MS (ES): m/z (MH⁺) 804.22 for C₄₈H₈₉N₃O₆. ¹H NMR (300 MHz, CDCl₃) δ: ppm 7.38 (br. s, 1H); 7.03 (br. s, 1H); 4.07 (t, 4H, J=6.0 Hz); 3.65 (br. s, 2H, J=6.0 Hz); 3.27 (d, 3H, J=6.0 Hz); 2.52 (br. t, 2H, J=6.0 Hz); 2.40 (br. t, 4H, J=6.0 Hz); 2.28 (t, 4H, J=6.0 Hz); 1.75 (br. pent., 2H, J=6.0 Hz); 1.67-1.51 (m, 8H); 1.47-1.17 (m, 46H); 0.93-0.82 (m, 12H).

### OTHER EMBODIMENTS

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims. All references described herein are incorporated by reference in their entireties.

What is claimed is:

1. A polynucleotide encoding a polypeptide (e.g., mRNA), wherein the polynucleotide comprises:
(a) a 5′-UTR comprising the sequence of SEQ ID NO: 1 or a variant or fragment thereof,
(b) a coding region comprising a stop element (e.g., as described herein); and
(c) a 3′-UTR (e.g., as described herein).

2. The polynucleotide of claim 1, wherein the 5′ UTR comprises a nucleic acid sequence of Formula A:
(SEQ ID NO: 46)
G G A A A U C G C A A A A (N₂)ₓ(N₃)ₓ
C U (N₄)ₓ(N₅)ₓ C G C G U U A G A U U
U C U U A G U U U U C U N₆ N₇ C A A C
U A G C A A G C U U U U U G U U C U
C G C C (N₈ C C)ₓ,
wherein:
(N₂)ₓ is a uracil and x is an integer from 0 to 5, e.g., wherein x=3 or 4;
(N₃)ₓ is a guanine and x is an integer from 0 to 1;
(N₄)ₓ is a cytosine and x is an integer from 0 to 1;
(N₅)ₓ is a uracil and x is an integer from 0 to 5, e.g., wherein x=2 or 3;
N₆ is a uracil or cytosine;
N₇ is a uracil or guanine; and/or
N₈ is a adenine or guanine and x is an integer from 0 to 1.

3. The polynucleotide of claim 1, wherein the variant of SEQ ID NO: 1 comprises a sequence with at least 50%, 60%, 70%, 80%, 85%, 90%, 95%, 96%, 97%, 98%, 99% or 100% identity to SEQ ID NO: 1, or a fragment thereof (e.g., a fragment that lacks the first one, two, three, four, five, six, or more nucleotides of SEQ ID NO: 1).

4. The polynucleotide of claim 1 or 3, wherein the variant of SEQ ID NO: 1 comprises a uridine content of at least 5%, 10%, 20%, 30%, 40%, 50%, 60%, 70%, or 80%.

5. The polynucleotide of any one of claims 1, or 3-4, wherein the variant of SEQ ID NO: 1 comprises at least 2, 3, 4, 5, 6, 7, 8, 9, 10, 11 or 12 consecutive uridines (e.g., a polyuridine tract).

6. The polynucleotide of claim 5, wherein the polyuridine tract in the variant of SEQ ID NO: 1 comprises at least at least 2-12, 2-11, 2-10, 2-9, 2-8, 2-7, 2-6, 2-5, 2-4, 2-3, 3-12, 4-12, 5-12, 6-12, 7-12, 8-12, 9-12, 10-12, 11-12, 2-6, or 3-5 consecutive uridines.

7. The polynucleotide of any one of claims 1 or 3-6, wherein the polyuridine tract in the variant of SEQ ID NO: 1 comprises 4 consecutive uridines.

8. The polynucleotide of any one of claims 1 or 3-7, wherein the polyuridine tract in the variant of SEQ ID NO: 1 comprises 5 consecutive uridines.

9. The polynucleotide of any one of claims 1 or 3-8, wherein the variant of SEQ ID NO: 1 comprises 1, 2, 3, 4, 5, 6, 7, 8, 9, 10, 11, 12, 13, 14, or 15 polyuridine tracts.

10. The polynucleotide of claim 9, wherein the variant of SEQ ID NO: 1 comprises 3 polyuridine tracts.

11. The polynucleotide of claim 9, wherein the variant of SEQ ID NO: 1 comprises 4 polyuridine tracts.

12. The polynucleotide of claim 9, wherein the variant of SEQ ID NO: 1 comprises 5 polyuridine tracts.

13. The polynucleotide of any one of claims 1 or 3-12, wherein one or more of the polyuridine tracts are adjacent to a different polyuridine tract.

14. The polynucleotide of any one of claims 1 or 3-13, wherein each of, e.g., all, the polyuridine tracts are adjacent to each other, e.g., all of the polyuridine tracts are contiguous.

15. The polynucleotide of any one of claims 1 or 3-14, wherein one or more of the polyuridine tracts are separated by 1, 2, 3, 4, 5, 6, 7, 8, 9, 10, 11, 2, 13, 14, 15, 16, 17, 18. 19, 20, 30, 40, 50 or 60 nucleotides.

16. The polynucleotide of any one of claims 1 or 3-15, wherein each of, e.g., all of, the polyuridine tracts are separated by 1, 2, 3, 4, 5, 6, 7, 8, 9, 10, 11, 2, 13, 14, 15, 16, 17, 18. 19, 20, 30, 40, 50 or 60 nucleotides.

17. The polynucleotide of any one of claims 1 or 3-16, wherein a first polyuridine tract and a second polyuridine tract are adjacent to each other.

18. The polynucleotide of any one of the preceding claims wherein the 5′ UTR comprises a Kozak sequence, e.g., a GCCRCC nucleotide sequence (SEQ ID NO: 43) wherein R is an adenine or guanine.

19. The polynucleotide of any one of the preceding claims, wherein the 5′ UTR comprises the sequence of SEQ ID NO: 1 or a sequence with at least 80%, 85%, 90%, 95%, 96%, 97%, 98%, 99% or 100% identity to SEQ ID NO: 1.

20. The polynucleotide of any one of the preceding claims, wherein the 5′ UTR comprises the sequence of SEQ ID NO: 41 or a sequence with at least 80%, 85%, 90%, 95%, 96%, 97%, 98%, 99% or 100% identity to SEQ ID NO: 41, or a fragment thereof (e.g., a fragment that lacks the first one, two, three, four, five, six, or more nucleotides of SEQ ID NO: 41).

21. The polynucleotide of any one of the preceding claims, wherein the 5′ UTR comprises the sequence of SEQ ID NO: 42 or a sequence with at least 80%, 85%, 90%, 95%, 96%, 97%, 98%, 99% or 100% identity to SEQ ID NO: 42, or a fragment thereof (e.g., a fragment that lacks the first one, two, three, four, five, six, or more nucleotides of SEQ ID NO: 42).

22. The polynucleotide of any one of the preceding claims, wherein the 5′ UTR results in an increased half-life of the polynucleotide, e.g., about 1.5-20-fold increase in half-life of the polynucleotide.

23. The polynucleotide of claim 22, wherein the increase in half-life of the polynucleotide is compared to an otherwise similar polynucleotide which does not have a 5′ UTR, has a different 5′ UTR, or does not have a 5′ UTR of any one of claims 1-21.

24. The polynucleotide of any one of claims 22-23, wherein the increase in half-life of the polynucleotide is measured according to an assay that measures the half-life of a polynucleotide, e.g., an assay described in any one of Examples disclosed herein.

25. The polynucleotide of any one of the preceding claims, wherein the 5′ UTR results in an increased level and/or activity, e.g., output, of the polypeptide encoded by the polynucleotide.

26. The polynucleotide of claim 25, wherein the 5′ UTR results in about 1.5-20-fold increase in level and/or activity, e.g., output, of the polypeptide encoded by the polynucleotide.

27. The polynucleotide of claim 26, wherein the increase in activity is compared to an otherwise similar polynucleotide which does not have a 5′ UTR, has a different 5′ UTR, or does not have the 5′ UTR of claim 1.

28. The polynucleotide of any one of the preceding claims, wherein the coding region of (b) comprises a stop element chosen from a stop element provided in Table 3, e.g., SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO:36, SEQ ID NO: 37, SEQ ID NO: 56, SEQ ID NO: 57, SEQ ID NO: 62, SEQ ID NO: 93 or SEQ ID NO: 96.

29. The polynucleotide of any one of the preceding claims, wherein the 3′ UTR of (c) comprises a 3′ UTR sequence provided in Table 2 or a sequence with at least 80%, 85%, 90%, 95%, 96%, 97%, 98%, 99% or 100% identity to a 3′ UTR sequence provided in Table 2, or a fragment thereof (e.g., a fragment that lacks the first one, two, three, four, five, six, or more nucleotides of the 3′ UTR sequence provided in Table 2)
optionally wherein the polynucleotide comprises a sequence with at least 80%, 85%, 90%, 95%, 96%, 97%, 98%, 99% or 100% identity to a sequence provided in Table 4.

30. The polynucleotide of claim 29, wherein the 3′ UTR comprises a sequence with at least 80%, 85%, 90%, 95%, 96%, 97%, 98%, 99% or 100% identity to SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 45, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 94 or SEQ ID NO: 95, or a fragment thereof (e.g., a fragment that lacks the first one, two, three, four, five, six, or more nucleotides of any of the aforesaid sequences), optionally, wherein the 3′ UTR comprises a micro RNA (miRNA) binding site, e.g., as described herein, e.g., a sequence of any one of SEQ ID NOs: 38-40, optionally wherein the 3′ UTR comprises a TENT recruiting sequence, e.g., as described herein, e.g., a sequence of SEQ ID NO: 91 or 92,
optionally wherein the polynucleotide comprises a sequence with at least 80%, 85%, 90%, 95%, 96%, 97%, 98%, 99% or 100% identity to any of SEQ ID NOs: 47, 48, 49, 50, 122, 52, 53, 54, 55, 59, 60, 61, 126, 127, 97, 98, 99, 100, 101, 102, 103, 104, 105, 106, 107, 108, 109, 110, 111, 112, 113, 114, 115, 116, 117, 118, 119, or 120, or a variant or fragment thereof.

31. The polynucleotide of any one of claims 1-27, wherein:
(i) the coding region of (b) comprises a stop element chosen from a stop element provided in Table 3, e.g., SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO:36, SEQ ID NO: 37, SEQ ID NO: 56, SEQ ID NO: 57, SEQ ID NO: 62, SEQ ID NO: 93 or SEQ ID NO: 96; and
(ii) the 3′ UTR of (c) comprises a 3′ UTR sequence provided in Table 2 or a sequence with at least 80%, 85%, 90%, 95%, 96%, 97%, 98%, 99% or 100% identity to a 3′ UTR sequence provided in Table 2, or a fragment thereof (e.g., a fragment that lacks the first one, two, three, four, five, six, or more nucleotides of the 3′ UTR sequence provided in Table 2)
optionally wherein the polynucleotide comprises a sequence with at least 80%, 85%, 90%, 95%, 96%, 97%, 98%, 99% or 100% identity to a sequence provided in Table 4, e.g., any of SEQ ID NOs: 47, 48, 49, 50, 122, 52, 53, 54, 55, 59, 60, 61, 126, 127, 97, 98, 99, 100, 101, 102, 103, 104, 105, 106, 107, 108, 109, 110, 111, 112, 113, 114, 115, 116, 117, 118, 119, or 120, or a variant or fragment thereof.

32. The polynucleotide of claim 31, wherein the 3′ UTR comprises a sequence with at least 80%, 85%, 90%, 95%, 96%, 97%, 98%, 99% or 100% identity to SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 45, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 94 or SEQ ID NO: 95, or a fragment thereof (e.g., a fragment that lacks the first one, two, three, four, five, six, or more nucleotides of any of the aforesaid sequences).

33. The polynucleotide of claim 32 or 33, wherein the 3′ UTR comprises a micro RNA binding site, e.g., as described herein, e.g., a sequence of any one of SEQ ID NOs: 38-40, and/or wherein the 3′ UTR comprises a TENT recruiting sequence, e.g., as described herein, e.g., a sequence of SEQ ID NO: 91 or 92.

34. A polynucleotide encoding a polypeptide, wherein the polynucleotide comprises:
(a) a 5′-UTR (e.g., as described herein);
(b) a coding region comprising a stop element chosen from a stop element provided in Table 3; and
(c) a 3′-UTR (e.g., as described herein).

35. The polynucleotide of claim 34, wherein the stop element comprises the sequence of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO:36, SEQ ID NO; 62, SEQ ID NO: 93 or SEQ ID NO: 96.

36. The polynucleotide of claim 34, wherein the coding region of (b) comprises a stop element comprising a consensus sequence of Formula B:
(SEQ ID NO: 37)
X₋₃-X₋₂-X₋₁-U-A-A-X₁-X₂-X₃-X₄-X₅-X₆-
X₇-X₈-X₉-X₁₀-X₁₁-X₁₂
wherein:
X₁ is a G or A;
X₂, X₄, X₅ X₆ or X₇ is each independently C or U;
X₃ is C or A;
X₈, X₁₀, X₁₁, X₁₂ X₋₁ or X₋₃ is each independently C or G;
X₉ is G or U; and/or
X₋₂ is A or U.

37. The polynucleotide of claim 34, wherein the coding region of (b) comprises a stop element comprising a consensus sequence of Formula C:
(SEQ ID NO: 56)
X₋₃-X₋₂-X₋₁-U-G-A-X₁-X₂-X₃-X₄-X₅-X₆-
X₇-X₈-X₉-X₁₀-X₁₁-X₁₂
wherein:
X₋₃, X₋₁, X₂, X₅, X₆, X₇, X₈, X₉, or X₁₂ is each independently G or C;
X₋₂, X₃, or X₄ is each independent A or C;
X₁ is A or G; and/or
X₁₀ or X₁₁ is each independently C or U.

38. The polynucleotide of claim 34, wherein the coding region of (b) comprises a stop element comprising a consensus sequence of Formula D
(SEQ ID NO: 57)
X₋₃-X₋₂-X₋₁-U-A-G-X₁-X₂-X₃-X₄-X₅-X₆-
X₇-X₈-X₉-X₁₀-X₁₁-X₁₂
wherein:
X₋₃, X₋₁, X₂, X₃, X₁₀ is each independently G or C;
X₋₂ or X₉ is each independently A or U;
X₁ or X₄ is each independently A or G;
X₅ or X₈ is each independently A or C; and/or
X₆, X₇, X₁₁ or X₁₂ is each independently C or U.

39. The polynucleotide of any one of claims 36-39, wherein the consensus sequence has a high GC content, e.g., GC content of about 50%, 60%, 70%, 80%, 90% or 99%.

40. The polynucleotide of any one of claims 34-39, wherein the stop element results in an increased half-life of the polynucleotide, e.g., about 1.5-20-fold increase in half-life of the polynucleotide.

41. The polynucleotide of claim 40, wherein the increase in half-life of the polynucleotide is compared to an otherwise similar polynucleotide which does not have a stop element, has a different stop element, or does not have the stop element of any one of claims 34-37.

42. The polynucleotide of claim 40 or 41, wherein the increase in half-life of the polynucleotide is measured according to an assay which measures the half-life of a polynucleotide, e.g., an assay described in any one of Examples disclosed herein.

43. The polynucleotide of any one of claims 34-42, wherein the stop element results in an increased level and/or activity, e.g., output or duration of expression, of the polypeptide encoded by the polynucleotide.

44. The polynucleotide of claim 43, wherein the increase in level and/or activity, e.g., output or duration of expression, of the polypeptide is measured according to an assay which measures the level and/or activity, e.g., output or duration of expression of a polypeptide, e.g., an assay described in any one of Examples disclosed herein.

45. The polynucleotide of claim 44, wherein the stop element results in about 1.5-20-fold increase in level and/or activity, e.g., output, of the polypeptide encoded by the polynucleotide.

46. The polynucleotide of claim 44, wherein the stop element results in about 1.5-20 fold increase in level and/or activity, e.g., detectable level or activity, of the polypeptide encoded by the polynucleotide for about 1, 2, 3, 4, 5, 6, 7, 8, 9, 10, or 14 days.

47. The polynucleotide of claim 46 wherein the stop element results in detectable level or activity of the polypeptide encoded by the polynucleotide for about 1, 2, 3, 4, 5, 6, 7, 8, 9, 10, or 14 days.

48. The polynucleotide of any one of claims 38-47, wherein the increase is compared to an otherwise similar polynucleotide which does not have a stop element, has a different stop element, or does not have the stop element of any one of claims 34-39.

49. The polynucleotide of any one of claims 34-48, wherein the 5′ UTR of (a) comprises a 5′ UTR sequence provided in Table 1 or a sequence with at least 80%, 85%, 90%, 95%, 96%, 97%, 98%, 99% or 100% identity to a 5′ UTR sequence provided in Table 1, or a variant or a fragment thereof (e.g., a fragment that lacks the first one, two, three, four, five, or six nucleotides of the 5′ UTR sequence provided in Table 1).

50. The polynucleotide of claim 49, wherein the 5′ UTR comprises a sequence with at least 80%, 85%, 90%, 95%, 96%, 97%, 98%, 99% or 100% identity to SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 41, SEQ ID NO: 42, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 88, SEQ ID NO: 89 or SEQ ID NO: 90, or a fragment thereof (e.g., a fragment that lacks the first one, two, three, four, five, or six nucleotides of any of the aforesaid sequences).

51. The polynucleotide of any one of claims 34-50, wherein the 3′ UTR of (c) comprises a 3′ UTR sequence provided in Table 2 or a sequence with at least 80%, 85%, 90%, 95%, 96%, 97%, 98%, 99% or 100% identity to a 3′ UTR sequence provided in Table 2, or a fragment thereof (e.g., a fragment that lacks the first one, two, three, four, five, six, or more nucleotides of the 3′ UTR sequence provided in Table 2), optionally wherein the polynucleotide comprises a sequence with at least 80%, 85%, 90%, 95%, 96%, 97%, 98%, 99% or 100% identity to a sequence provided in Table 4.

52. The polynucleotide of claim 51, wherein the 3′ UTR comprises a sequence with at least 80%, 85%, 90%, 95%, 96%, 97%, 98%, 99% or 100% identity to SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 45, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 94 or SEQ ID NO: 95, or a fragment thereof (e.g., a fragment that lacks the first one, two, three, four, five, six, or more nucleotides of any of the aforesaid sequences),
optionally wherein the polynucleotide comprises a sequence with at least 80%, 85%, 90%, 95%, 96%, 97%, 98%, 99% or 100% identity to any of SEQ ID NOs: 47, 48, 49, 50, 122, 52, 53, 54, 55, 59, 60, 61, 126, 127, 97, 98, 99, 100, 101, 102, 103, 104, 105, 106, 107, 108, 109, 110, 111, 112, 113, 114, 115, 116, 117, 118, 119, or 120, or a variant or fragment thereof.

53. The polynucleotide of claim 52, wherein the 3′ UTR comprises a micro RNA binding site, e.g., as described herein, e.g., a sequence of any one of SEQ ID NOs: 38-40, and/or wherein the 3′ UTR comprises a TENT recruiting sequence, e.g., as described herein, e.g., a sequence of SEQ ID NO: 91 or 92.

54. The polynucleotide of any one of claims 34-53, wherein:
(i) the 5′ UTR of (a) comprises a 5′ UTR sequence provided in Table 1 or a sequence with at least 80%, 85%, 90%, 95%, 96%, 97%, 98%, 99% or 100% identity to a 3′ UTR sequence provided in Table 1, or a fragment thereof (e.g., a fragment that lacks the first one, two, three, four, five, or six nucleotides of the 5′ UTR sequence provided in Table 1); and
(ii) the 3′ UTR of (c) comprises a 3′ UTR sequence provided in Table 2 or a sequence with at least 80%, 85%, 90%, 95%, 96%, 97%, 98%, 99% or 100% identity to a 3′ UTR sequence provided in Table 2, or a fragment thereof (e.g., a fragment that lacks the first one, two, three, four, five, six, or more nucleotides of the 3′ UTR sequence provided in Table 2)
optionally wherein the polynucleotide comprises a sequence with at least 80%, 85%, 90%, 95%, 96%, 97%, 98%, 99% or 100% identity to a sequence provided in Table 4.

55. The polynucleotide of claim 54, wherein the 5′ UTR comprises a sequence with at least 80%, 85%, 90%, 95%, 96%, 97%, 98%, 99% or 100% identity to SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 41, SEQ ID NO: 42, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 88, SEQ ID NO: 89 or SEQ ID NO: 90, or a fragment thereof (e.g., a fragment that lacks the first one, two, three, four, five, or six nucleotides of any of the aforesaid sequences),
optionally wherein the polynucleotide comprises a sequence with at least 80%, 85%, 90%, 95%, 96%, 97%, 98%, 99% or 100% identity to any of SEQ ID NOs: 47, 48, 49, 50, 122, 52, 53, 54, 55, 59, 60, 61, 126, 127, 97, 98, 99, 100, 101, 102, 103, 104, 105, 106, 107, 108, 109, 110, 111, 112, 113, 114, 115, 116, 117, 118, 119, or 120, or a variant or fragment thereof.

56. The polynucleotide of claim 54 or 55, wherein the 3′ UTR comprises a sequence with at least 80%, 85%, 90%, 95%, 96%, 97%, 98%, 99% or 100% identity to SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 45, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 94 or SEQ ID NO: 95, or a fragment thereof (e.g., a fragment that lacks the first one, two, three, four, five, six, or more nucleotides of any of the aforesaid sequences).

57. The polynucleotide of claim 56, wherein the 3′ UTR comprises a micro RNA binding site, e.g., as described herein, e.g., a sequence of any one of SEQ ID NOs: 38-40, and/or wherein the 3′ UTR comprises a TENT recruiting sequence, e.g., as described herein, e.g., a sequence of SEQ ID NO: 91 or 92.

58. A polynucleotide encoding a polypeptide, wherein the polynucleotide comprises:
(a) a 5′-UTR (e.g., as described herein);
(b) a coding region comprising a stop element (e.g., as described herein); and
(c) a 3′ UTR comprising a core sequence with at least 80%, 85%, 90%, 95%, 96%, 97%, 98%, 99% or 100% identity to SEQ ID NO: 11 or a fragment thereof.

59. The polynucleotide of claim 58, wherein the 3′ UTR core sequence is disposed immediately downstream of the stop element of (b).

60. The polynucleotide of claim 58, wherein the 3′ UTR core sequence is disposed at the C terminus end of the polynucleotide.

61. The polynucleotide of any one of claims 58-60, wherein the 3′ UTR comprising a core sequence comprises a first flanking sequence.

62. The polynucleotide of any one of claims 58-61, wherein the 3′ UTR comprising a core sequence comprises a second flanking sequence.

63. The polynucleotide of claim 61 or 62, wherein the 3′ UTR comprising a core sequence comprises a first flanking sequence and a second flanking sequence.

64. The polynucleotide of any one of claims 61-63, wherein the first flanking sequence comprises a sequence of about 5-25, about 5-20, about 5-15, about 5-10, about 10-25, about 15-25, about 20-25 nucleotides.

65. The polynucleotide of any one of claims 61-64, wherein the first flanking sequence comprises a sequence of about 5, 6, 7, 8, 9, 10, 11, 12, 1 3, 14, 15, 16, 17, 18, 19, 20, 21, 22, 23, 24, or 25 nucleotides, e.g., 11 nucleotides.

66. The polynucleotide of any one of claims 61-65, wherein the second flanking sequence comprises a sequence of about 20-80, about 20-75, about 20-70, about 20-65, about 20-60, about 20-55, about 20-50, about 20-45, bout 20-40, about 20-35, about 20-30, about 20-25, about 25-80, about 30-80, about 35-80, about 40-80, about 45-80, about 50-80, about 55-80, about 60-80, about 65-80, about 70-80 or about 75-80 nucleotides.

67. The polynucleotide of any one of claims 61-66, wherein the second flanking sequence comprises a sequence of about 20, 21, 22, 23, 2 4, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, or 80 nucleotides, e.g., 39 nucleotides.

68. The polynucleotide of any one of claims 61-67, wherein the first flanking sequence is upstream or downstream of the core sequence.

69. The polynucleotide of any one of claims 61-67, wherein the second flanking sequence is upstream or downstream of the core sequence.

70. The polynucleotide of any one of claims 58-69, wherein the 3′ UTR comprises a fragment of SEQ ID NO: 11, e.g., a 5 nucleotide (nt), 10 nt, 15 nt, 20 nt, 25 nt, 30 nt, 35 nt, 40 nt, 45 nt, 50 nt, 55 nt, 60 nt, 65 nt, or 70 nt fragment of SEQ ID NO: 11.

71. The polynucleotide of any one of claims 58-70, wherein the 3′ UTR comprises 15-25 nt fragment comprising a 60 nt fragment of SEQ ID NO: 11.

72. The polynucleotide of any one of claims 58-71, wherein the 3′ UTR comprises the sequence of SEQ ID NO: 45 or a fragment thereof (e.g., a fragment that lacks the first one, two, three, four, five, six, or more nucleotides of SEQ ID NO: 45).

73. The polynucleotide of any one of claims 58-71, wherein the 3′ UTR comprises the sequence of SEQ ID NO: 11 or a fragment thereof (e.g., a fragment that lacks the first one, two, three, four, five, six, or more nucleotides of SEQ ID NO: 11).

74. The polynucleotide of any one of claims 58-73, wherein the 3′ UTR results in an increased half-life of the polynucleotide, e.g., about 1.5-10 fold increase in half-life of the polynucleotide, e.g., as measured by an assay that measures the half-life of a polynucleotide, e.g., an assay of any one of Examples disclosed herein.

75. The polynucleotide of claim 74, wherein the 3′ UTR results in a polynucleotide with a mean half-life score of greater than 10.

76. The polynucleotide of any one of claims 58-73, wherein the 3′ UTR results in an increased level and/or activity, e.g., output, of the polypeptide encoded by the polynucleotide.

77. The polynucleotide of any one of claims 74-76, wherein the increase is compared to an otherwise similar polynucleotide which does not have a 3′ UTR, has a different 3′ UTR, or does not have the 3′ UTR of claim 76.

78. The polynucleotide of any one of claims 58-77, wherein the 3′ UTR comprises a micro RNA (miRNA) binding site, e.g., as described herein, and/or a TENT recruiting sequence, e.g., as described herein.

79. The polynucleotide of claim 78, wherein the 3′ UTR comprises a miRNA binding site of SEQ ID NO: 38, SEQ ID NO: 39, SEQ ID NO: 40 or a combination thereof, and/or a TENT recruiting sequence comprising the sequence of SEQ ID NO: 91 or 92.

80. The polynucleotide of claim 78 or 79, wherein the 3′ UTR comprises a plurality of miRNA binding sites, e.g., 2, 3, 4, 5, 6, 7 or 8 miRNA binding sites.

81. The polynucleotide of claim 80, wherein the plurality of miRNA binding sites comprises the same or different miRNA binding sites.

82. The polynucleotide of any one of claims 58-81, wherein the 5′ UTR of (a) comprises a 5′ UTR sequence provided in Table 1 or a sequence with at least 80%, 85%, 90%, 95%, 96%, 97%, 98%, 99% or 100% identity to a 5′ UTR sequence provided in Table 1, or a fragment thereof (e.g., a fragment that lacks the first one, two, three, four, five, or six nucleotides of the 5′ UTR sequence provided in Table 1).

83. The polynucleotide of claim 82, wherein the 5′ UTR comprises a sequence with at least 80%, 85%, 90%, 95%, 96%, 97%, 98%, 99% or 100% identity to SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 41, SEQ ID NO: 42, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 88, SEQ ID NO: 89 or SEQ ID NO: 90, or a fragment thereof (e.g., a fragment that lacks the first one, two, three, four, five, or six nucleotides of any of the aforesaid sequences).

84. The polynucleotide of any one of claims 58-83, wherein the coding region of (b) comprises a stop element sequence provided in Table 3.

85. The polynucleotide of claim 84, wherein the coding region of (b) comprises a stop element chosen from a stop element provided in Table 3, e.g., SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO:36 or SEQ ID NO: 37.

86. The polynucleotide of any one of claims 58-81, wherein:
(i) the 5′ UTR of (a) comprises a 5′ UTR sequence provided in Table 1 or a sequence with at least 80%, 85%, 90%, 95%, 96%, 97%, 98%, 99% or 100% identity to a 3′ UTR sequence provided in Table 1, or a fragment thereof (e.g., a fragment that lacks the first one, two, three, four, five, or six nucleotides of the 5′ UTR sequence provided in Table 1); and
(ii) the stop element of (b) comprises a stop element sequence provided in Table 3.

87. The polynucleotide of claim 86, wherein the 5′ UTR comprises a sequence with at least 80%, 85%, 90%, 95%, 96%, 97%, 98%, 99% or 100% identity to SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 41, SEQ ID NO: 42, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 88, SEQ ID NO: 89 or SEQ ID NO: 90, or a fragment thereof (e.g., a fragment that lacks the first one, two, three, four, five, or six nucleotides of any of the aforesaid sequences).

88. The polynucleotide of any one of claims 58-81 or 86, wherein the coding region of (b) comprises a stop element chosen from a stop element provided in Table 3, e.g., SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 56, SEQ ID NO: 57, SEQ ID NO; 62, SEQ ID NO: 93 or SEQ ID NO: 96.

89. The polynucleotide of any one of the preceding claims, wherein the coding region of the polynucleotide comprises a sequence encoding a therapeutic payload or a prophylactic payload.

90. The polynucleotide of claim 89, wherein the therapeutic payload or prophylactic payload comprises a secreted protein, a membrane-bound protein; or an intercellular protein.

91. The polynucleotide of claim 90, wherein the therapeutic payload or prophylactic payload is chosen from a cytokine, an antibody, a vaccine (e.g., an antigen, an immunogenic epitope), a receptor, an enzyme, a hormone, a transcription factor, a ligand, a membrane transporter, a structural protein, a nuclease, or a component, variant or fragment (e.g., a biologically active fragment) thereof.

92. The polynucleotide of any one of claims 89-91, wherein the therapeutic payload or prophylactic payload comprises a protein or peptide.

93. The polynucleotide of any one of the preceding claims, further comprising at least one 5′ cap structure, e.g., as described herein, and/or a poly A tail, e.g., as described herein, optionally wherein the poly A tail comprises one or more non-adenosine residues, e.g., one or more guanosines.

94. The polynucleotide of claim 93, wherein the 5′ cap structure comprises a sequence of GG, GA, or GGA, wherein the underlined, italicized G is an inverted G nucleotide followed by a 5′-5′-triphosphate group.

95. The polynucleotide of any one of the preceding claims, further comprising a 3′ stabilizing region, e.g., a stabilized tail e.g., as described herein.

96. The polynucleotide of claim 95, wherein the 3′ stabilizing region comprises a poly A tail, e.g., a poly A tail comprising 80-150, e.g., 120, adenine (SEQ ID NO: 123), optionally wherein the poly A tail comprises one or more non-adenosine residues, e.g., one or more guanosines.

97. The polynucleotide of claim 95 or 96, wherein the poly A tail comprises a UCUAG sequence (SEQ ID NO: 44).

98. The polynucleotide of claim 97, wherein the poly A tail comprises about 80-120, e.g., 100, adenines upstream of SEQ ID NO: 44.

99. The polynucleotide of claim 97 or 98, wherein the poly A tail comprises about 1-40, e.g., 20, adenines downstream of SEQ ID NO: 44.

100. The polynucleotide of any one of claims 95-99, wherein the 3′ stabilizing region comprises at least one alternative nucleoside, optionally wherein the alternative nucleoside is an inverted thymidine (idT).

101. The polynucleotide of any one of claims 95-100, wherein the 3′ stabilizing region comprises a structure of Formula VII: 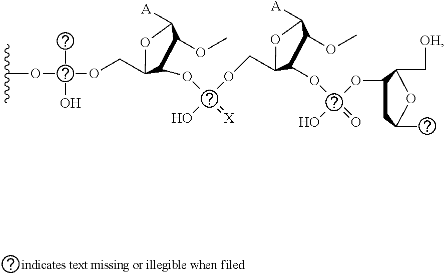
or a salt thereof, wherein each X is independently O or S, and A represents adenine and T represents thymine.

102. The polynucleotide of any one of the preceding claims, wherein the polynucleotide comprises an mRNA.

103. The polynucleotide of claim 102, wherein the mRNA comprises at least one chemical modification.

104. The polynucleotide of claim 102 or 103, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

105. A lipid nanoparticle (LNP) composition comprising a polynucleotide of any one of the preceding claims.

106. A pharmaceutical composition comprising the LNP composition of claim 105.

107. A cell comprising the LNP composition of claim 105 or 106.

108. A method of increasing expression of a payload, e.g., a therapeutic payload or a prophylactic payload in a cell, comprising administering to the cell the LNP composition of claim 105 or 106.

109. A method of delivering the LNP composition of claim 105 or 106, to a cell.

110. The method of claim 109, comprising contacting the cell in vitro, in vivo or ex vivo with the LNP composition.

111. A method of delivering an LNP composition of claim 105 or 106, to a subject having a disease or disorder, e.g., as described herein.

112. A method of modulating an immune response in a subject, comprising administering to the subject in need thereof an effective amount of an LNP composition of claim 105 or 106.

113. A method of treating, preventing, or preventing a symptom of, a disease or disorder comprising administering to a subject in need thereof an effective amount of an LNP composition of claim 105 or 106.

114. The method, or the LNP composition of any one of claims 105-113, wherein the LNP composition comprises: (i) an ionizable lipid, e.g., an amino lipid; (ii) a sterol or other structural lipid; (iii) a non-cationic helper lipid or phospholipid; and (iv) a PEG-lipid.

115. The method, or the LNP composition of claim 114, wherein the ionizable lipid comprises an amino lipid.

116. The method, or the LNP composition of claim 114 or 115, wherein the ionizable lipid comprises a compound of any of Formulae (I), (IA), (IB), (IC), (II), (IIa), (IIb), (IIc), (IId), (IIe), (IIf), (IIg), (III), (IIIa1), (IIIa2), (IIIa3), (IIIa4), (IIIa5), (IIIa6), (IIIa7), or (IIIa8).

117. The method, or the LNP composition of any one of claims 105-116, wherein the ionizable lipid comprises a compound of Formula (I), a compound of Formula (IIa), or a compound of Formula (IIe).

118. The method, or the LNP composition of any one of claims 105-117, wherein the non-cationic helper lipid or phospholipid comprises a compound selected from the group consisting of DSPC, DPPC, or DOPC.

119. The method, or the LNP composition of any one of claims 105-118, wherein the phospholipid is DSPC, e.g., a variant of DSPC, e.g., a compound of Formula (IV).

120. The method, or the LNP composition of any one of claims 105-119, wherein the structural lipid is chosen from alpha-tocopherol, β-sitosterol or cholesterol.

121. The method, or the LNP composition of any one of claims 105-120, wherein the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

122. The method, or the LNP composition of any one of claims 105-121, wherein the PEG lipid is selected from the group consisting of PEG-c-DOMG, PEG-DMG, PEG-DLPE, PEG-DMPE, PEG-DPPC and PEG-DSPE lipid.

123. The method, or the LNP composition of any one of claims 105-121, wherein the PEG lipid is a compound chosen from: Formula (V), Formula (VI-A), Formula (VI-B), Formula (VI-C) or Formula (VI-D).

124. The method, or the LNP composition of any one of claims 105-123, wherein the LNP comprises a molar ratio of about 20-60% ionizable lipid:5-25% phospholipid:25-55% cholesterol; and 0.5-15% PEG lipid.

125. The method, or the LNP composition of any one of claims 105-124, wherein the LNP is formulated for intravenous, subcutaneous, intramuscular, intranasal, intraocular, rectal, pulmonary or oral delivery.

126. The method, or the LNP composition of any one of claims 105-125 wherein the subject is a mammal, e.g., a human.

127. The method, or the LNP composition of any one of claims 105-126, wherein the subject has a disease or disorder disclosed herein.


SEQUENCE LISTING (130 sequences)

SEQ ID NO: 1 - RNA, 75 nt, Artificial Sequence
  source: /note=“Description of Artificial Sequence: Synthetic oligonucleotide”
ggaaaucgcaaaauuugcucuucgcguuagauuucuuuuaguuuucucgcaacuagcaagcuuuuuguucucgcc

SEQ ID NO: 2 - RNA, 203 nt, Artificial Sequence
  source: /note=“Description of Artificial Sequence: Synthetic polynucleotide”
ggaaauccccacaaccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 3 - RNA, 76 nt, Artificial Sequence
  source: /note=“Description of Artificial Sequence: Synthetic oligonucleotide”
ggaaauaagagagaaaagaagaguaagaagaaauauaagaucucccugagcuucagggagccccggcgccgccacc

SEQ ID NO: 4 - RNA, 89 nt, Artificial Sequence
  source: /note=“Description of Artificial Sequence: Synthetic oligonucleotide”
ggaaaccccccacccccguaagagagaaaagaagaguaagaagaaauauaagaucucccugagcuucagggagccccggcgccgccacc

SEQ ID NO: 5 - RNA, 59 nt, Artificial Sequence
  source: /note=“Description of Artificial Sequence: Synthetic oligonucleotide”
ggagaacuuccgcuuccguuggcgcaagcgcuuucauuuuuucugcuaccgugacuaag

SEQ ID NO: 6 - RNA, 46 nt, Artificial Sequence
  source: /note=“Description of Artificial Sequence: Synthetic oligonucleotide”
ggaaauaagagagaaaagaagaguaagaagaaauauaagagccacc

SEQ ID NO: 7 - skipped

SEQ ID NO: 8 - RNA, 56 nt, Artificial Sequence
  source: /note=“Description of Artificial Sequence: Synthetic oligonucleotide”
ggaaauaagagagaaaagaagaguaagaagaaauauaagaccccggcgccgccacc

SEQ ID NO: 9-10 - skipped (2 entries)

SEQ ID NO: 11 - RNA, 110 nt, Artificial Sequence
  source: /note=“Description of Artificial Sequence: Synthetic polynucleotide”
gcuggagccuccugagagaccugugugaacuauugagaagaucggaacagcuccuuacucugaggaaguugguacccccguggucuuugaauaaagucugagugggcggc

SEQ ID NO: 12 - RNA, 110 nt, Artificial Sequence
  source: /note=“Description of Artificial Sequence: Synthetic polynucleotide”
gcuggagccucacucuccucuccaucccguauccaggcugugaauuuuucaaggaauauaaagaucgggauguacccccguggucuuugaauaaagucugagugggcggc

SEQ ID NO: 13 - RNA, 110 nt, Artificial Sequence
  source: /note=“Description of Artificial Sequence: Synthetic polynucleotide”
gcuggagccucuagugacggcaacagggcuugguuuuuccuuguugugaaaucgacaucucugaagacaggguacccccguggucuuugaauaaagucugagugggcggc

SEQ ID NO: 14 - RNA, 110 nt, Artificial Sequence
  source: /note=“Description of Artificial Sequence: Synthetic polynucleotide”
gcuggagccuccuuccaucuagucacaaagacuccuucguccccaguugccgucuaggauugggccucccaguacccccguggucuuugaauaaagucugagugggcggc

SEQ ID NO: 15 - RNA, 110 nt, Artificial Sequence
  source: /note=“Description of Artificial Sequence: Synthetic polynucleotide”
gcuggagccucccauaacaugacauaucuggauuuugugcuuagaaccuuaaauuggaagcauucuuaauuguacccccguggucuuugaauaaagucugagugggcggc

SEQ ID NO: 16 - RNA, 110 nt, Artificial Sequence
  source: /note=“Description of Artificial Sequence: Synthetic polynucleotide”
gcuggagccuccggaaaacuaaaauagagauauuucaagauuuuauaauuuucaaagaccuuugaaauauuguacccccguggucuuugaauaaagucugagugggcggc

SEQ ID NO: 17 - RNA, 110 nt, Artificial Sequence
  source: /note=“Description of Artificial Sequence: Synthetic polynucleotide”
gcuggagccucuacacauugcuucuaguuggcagaaauaauugauuaaaagaccagaaacugugauaacugguacccccguggucuuuaaauaaagucuaagugggcggc

SEQ ID NO: 18 - RNA, 110 nt, Artificial Sequence
  source: /note=“Description of Artificial Sequence: Synthetic polynucleotide”
gcuggagccucgguggccaugcuucuugccccuugggccuccccccagccccuccuccccuuccugcacccguacccccguggucuuugaauaaagucugagugggcggc

SEQ ID NO: 19 - RNA, 110 nt, Artificial Sequence
  source: /note=“Description of Artificial Sequence: Synthetic polynucleotide”
gcuggagccucgguggccuagcuucuugccccuugggccuccccccagccccuccuccccuuccugcacccguacccccguggucuuugaauaaagucugagugggcggc

SEQ ID NO: 20 - RNA, 132 nt, Artificial Sequence
  source: /note=“Description of Artificial Sequence: Synthetic polynucleotide”
gcuggagccucgguggccaugcuucuugccccuugggccuccccccagccccuccuccccuuccugcacccguaccccccaaacaccauugucacacuccaguggucuuugaauaaagucugagugggcggc

SEQ ID NO: 21 - RNA, 132 nt, Artificial Sequence
  source: /note=“Description of Artificial Sequence: Synthetic polynucleotide”
gcuggagccucgguggccuagcuucuugccccuugggccuccccccagccccuccuccccuuccugcacccguaccccccaaacaccauugucacacuccaguggucuuugaauaaagucugagugggcggc

SEQ ID NO: 22 - RNA, 133 nt, Artificial Sequence
  source: /note=“Description of Artificial Sequence: Synthetic polynucleotide”
gcuggagccucgguggccuagcuucuugccccuugggccuccccccagccccuccuccccuuccugcacccguacccccuccauaaaguaggaaacacuacaguggucuuugaauaaagucugagugggcggc

SEQ ID NO: 23 - RNA, 132 nt, Artificial Sequence
  source: /note=“Description of Artificial Sequence: Synthetic polynucleotide”
gcuggagccucgguggccuagcuucuugccccuugggccuccccccagccccuccuccccuuccugcacccguaccccccgcauuauuacucacgguacgaguggucuuugaauaaagucugagugggcggc

SEQ ID NO: 24 - RNA, 155 nt, Artificial Sequence
  source: /note=“Description of Artificial Sequence: Synthetic polynucleotide”
uccauaaaguaggaaacacuacagcuggagccucgguggccaugcuucuugccccuugggccuccccccagccccuccuccccuuccugcacccguaccccccgcauuauuacucacgguacgaguggucuuugaauaaagucugagugggcggc

SEQ ID NO: 25 - RNA, 179 nt, Artificial Sequence
  source: /note=“Description of Artificial Sequence: Synthetic polynucleotide”
uccauaaaguaggaaacacuacagcuggagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 26 - RNA, 9 nt, Artificial Sequence
  source: /note=“Description of Artificial Sequence: Synthetic oligonucleotide”
ugauaauag

SEQ ID NO: 27 - RNA, 9 nt, Artificial Sequence
  source: /note=“Description of Artificial Sequence: Synthetic oligonucleotide”
uaauaguaa

SEQ ID NO: 28 - RNA, 9 nt, Artificial Sequence
  source: /note=“Description of Artificial Sequence: Synthetic oligonucleotide”
uaagucuaa

SEQ ID NO: 29 - RNA, 9 nt, Artificial Sequence
  source: /note=“Description of Artificial Sequence: Synthetic oligonucleotide”
uaaagcuaa

SEQ ID NO: 30 - RNA, 9 nt, Artificial Sequence
  source: /note=“Description of Artificial Sequence: Synthetic oligonucleotide”
uaagucucc

SEQ ID NO: 31 - RNA, 9 nt, Artificial Sequence
  source: /note=“Description of Artificial Sequence: Synthetic oligonucleotide”
uaaggcuaa

SEQ ID NO: 32 - RNA, 15 nt, Artificial Sequence
  source: /note=“Description of Artificial Sequence: Synthetic oligonucleotide”
uaagccccuccgggg

SEQ ID NO: 33 - RNA, 15 nt, Artificial Sequence
  source: /note=“Description of Artificial Sequence: Synthetic oligonucleotide”
uaaagcuccccgggg

SEQ ID NO: 34 - RNA, 9 nt, Artificial Sequence
  source: /note=“Description of Artificial Sequence: Synthetic oligonucleotide”
uaagccccu

SEQ ID NO: 35 - RNA, 9 nt, Artificial Sequence
  source: /note=“Description of Artificial Sequence: Synthetic oligonucleotide”
uaaagcucc

SEQ ID NO: 36 - RNA, 9 nt, Artificial Sequence
  source: /note=“Description of Artificial Sequence: Synthetic oligonucleotide”
uaggguuaa

SEQ ID NO: 37 - RNA, 18 nt, Artificial Sequence
  source: /note=“Description of Artificial Sequence: Synthetic oligonucleotide”
swsuaarymyyyysksss

SEQ ID NO: 38 - RNA, 22 nt, Artificial Sequence
  source: /note=“Description of Artificial Sequence: Synthetic oligonucleotide”
caaacaccauugucacacucca

SEQ ID NO: 39 - RNA, 23 nt, Artificial Sequence
  source: /note=“Description of Artificial Sequence: Synthetic oligonucleotide”
uccauaaaguaggaaacacuaca

SEQ ID NO: 40 - RNA, 22 nt, Artificial Sequence
  source: /note=“Description of Artificial Sequence: Synthetic oligonucleotide”
cgcauuauuacucacgguacga

SEQ ID NO: 41 - RNA, 81 nt, Artificial Sequence
  source: /note=“Description of Artificial Sequence: Synthetic oligonucleotide”
ggaaaucgcaaaauuugcucuucgcguuagauuucuuuuaguuuucucgcaacuagcaagcuuuuuguucucgccgccgcc

SEQ ID NO: 42 - RNA, 81 nt, Artificial Sequence
  source: /note=“Description of Artificial Sequence: Synthetic oligonucleotide”
ggaaaucgcaaaauuuucuuuucgcguuagauuucuuuuaguuuucuuucaacuagcaagcuuuuuguucucgccgccgcc

SEQ ID NO: 43 - RNA, 6 nt, Artificial Sequence
  source: /note=“Description of Artificial Sequence: Synthetic oligonucleotide”
gccrcc

SEQ ID NO: 44 - RNA, 5 nt, Artificial Sequence
  source: /note=“Description of Artificial Sequence: Synthetic oligonucleotide”
ucuag

SEQ ID NO: 45 - RNA, 60 nt, Artificial Sequence
  source: /note=“Description of Artificial Sequence: Synthetic oligonucleotide”
cugagagaccugugugaacuauugagaagaucggaacagcuccuuacucugaggaaguug

SEQ ID NO: 46 - RNA, 83 nt, Artificial Sequence
  source: /note=“Description of Artificial Sequence: Synthetic oligonucleotide”
  misc_feature (14)..(18): /note=“This region may encompass 0 to 5 nucleotides”
  misc_feature (19)..(19): /note=“May or may not be present”
  misc_feature (22)..(22): /note=“May or may not be present”
  misc_feature (23)..(27): /note=“This region may encompass 0 to 5 nucleotides”
  misc_feature (81)..(83): /note=“May or may not be present”
ggaaaucgcaaaauuuuugcucuuuuucgcguuagauuucuuuuaguuuucuykcaacuagcaagcuuuuuguucucgccrcc

SEQ ID NO: 47 - RNA, 119 nt, Artificial Sequence
  source: /note=“Description of Artificial Sequence: Synthetic polynucleotide”
uaggguuaagcuggagccucgguggccuagcuucuugccccuugggccuccccccagccccuccuccccuuccugcacccguacccccguggucuuugaauaaagucugagugggcggc

SEQ ID NO: 48 - RNA, 119 nt, Artificial Sequence
  source: /note=“Description of Artificial Sequence: Synthetic polynucleotide”
uaaagcuccgcuggagccucgguggccuagcuucuugccccuugggccuccccccagccccuccuccccuuccugcacccguacccccguggucuuugaauaaagucugagugggcggc

SEQ ID NO: 49 - RNA, 119 nt, Artificial Sequence
  source: /note=“Description of Artificial Sequence: Synthetic polynucleotide”
uaagccccugcuggagccucgguggccuagcuucuugccccuugggccuccccccagccccuccuccccuuccugcacccguacccccguggucuuugaauaaagucugagugggcggc

SEQ ID NO: 50 - RNA, 119 nt, Artificial Sequence
  source: /note=“Description of Artificial Sequence: Synthetic polynucleotide”
uaagcacccgcuggagccucgguggccuagcuucuugccccuugggccuccccccagccccuccuccccuuccugcacccguacccccguggucuuugaauaaagucugagugggcggc

SEQ ID NO: 51 - RNA, 120 nt, Artificial Sequence
  source: /note=“Description of Artificial Sequence: Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 52 - RNA, 119 nt, Artificial Sequence
  source: /note=“Description of Artificial Sequence: Synthetic polynucleotide”
uaaggcuaagcuggagccucgguggccuagcuucuugccccuugggccuccccccagccccuccuccccuuccugcacccguacccccguggucuuugaauaaagucugagugggcggc

SEQ ID NO: 53 - RNA, 119 nt, Artificial Sequence
  source: /note=“Description of Artificial Sequence: Synthetic polynucleotide”
uaagucuccgcuggagccucgguggccuagcuucuugccccuugggccuccccccagccccuccuccccuuccugcacccguacccccguggucuuugaauaaagucugagugggcggc

SEQ ID NO: 54 - RNA, 119 nt, Artificial Sequence
  source: /note=“Description of Artificial Sequence: Synthetic polynucleotide”
uaaagcuaagcuggagccucgguggccuagcuucuugccccuugggccuccccccagccccuccuccccuuccugcacccguacccccguggucuuugaauaaagucugagugggcggc

SEQ ID NO: 55 - RNA, 119 nt, Artificial Sequence
  source: /note=“Description of Artificial Sequence: Synthetic polynucleotide”
uaagucuaagcuggagccucgguggccuagcuucuugccccuugggccuccccccagccccuccuccccuuccugcacccguacccccguggucuuugaauaaagucugagugggcggc

SEQ ID NO: 56 - RNA, 18 nt, Artificial Sequence
  source: /note=“Description of Artificial Sequence: Synthetic oligonucleotide”
smsugarsmmsssssyys

SEQ ID NO: 57 - RNA, 18 nt, Artificial Sequence
  source: /note=“Description of Artificial Sequence: Synthetic oligonucleotide”
swsuagrssrmyymwsyy

SEQ ID NO: 58 - skipped

SEQ ID NO: 59 - RNA, 185 nt, Artificial Sequence
  source: /note=“Description of Artificial Sequence: Synthetic polynucleotide”
uaaagcuccauaaaguaggaaacacuacagcuggagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 60 - RNA, 188 nt, Artificial Sequence
  source: /note=“Description of Artificial Sequence: Synthetic polynucleotide”
uaagccccuccgggguccauaaaguaggaaacacuacagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 61 - RNA, 188 nt, Artificial Sequence
  source: /note=“Description of Artificial Sequence: Synthetic polynucleotide”
uaaagcuccccgggguccauaaaguaggaaacacuacagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 62 - RNA, 9 nt, Artificial Sequence
  source: /note=“Description of Artificial Sequence: Synthetic oligonucleotide”
uaagcaccc

SEQ ID NO: 63 - RNA, 79 nt, Artificial Sequence
  source: /note=“Description of Artificial Sequence: Synthetic oligonucleotide”
ggaaacuuuauuuaguguuacuuuauuuucuguuuauuuguguuucuucaguggguuuguucuaauuuccuuggccgcc

SEQ ID NO: 64 - RNA, 79 nt, Artificial Sequence
  source: /note=“Description of Artificial Sequence: Synthetic oligonucleotide”
ggaaaaucuguauuagguuggcguguucuuuggucgguuguuaguauuguuguugauucguuuguggucgguugccgcc

SEQ ID NO: 65 - RNA, 79 nt, Artificial Sequence
  source: /note=“Description of Artificial Sequence: Synthetic oligonucleotide”
ggaaaauuauuaacaucuugguauucucgauaaccauucguuggauuuuauuguauucguaguuuggguuccugccgcc

SEQ ID NO: 66 - RNA, 79 nt, Artificial Sequence
  source: /note=“Description of Artificial Sequence: Synthetic oligonucleotide”
ggaaauuauuauuauuucuagcuacaauuuaucauuguauuauuuuagcuauucaucauuauuuacuuggugaucaaca

SEQ ID NO: 67 - RNA, 79 nt, Artificial Sequence
  source: /note=“Description of Artificial Sequence: Synthetic oligonucleotide”
ggaaauagguuguuaaccaaguucaagccuaauaagcuuggauucuggugacuugcuucaccguuggcgggcaccgauc

SEQ ID NO: 68 - RNA, 74 nt, Artificial Sequence
  source: /note=“Description of Artificial Sequence: Synthetic oligonucleotide”
ggaaaucguagagagucguacuuaguacauaucgacuaucgguggacaccaucaagauuauaaaccaggccaga

SEQ ID NO: 69 - RNA, 79 nt, Artificial Sequence
  source: /note=“Description of Artificial Sequence: Synthetic oligonucleotide”
ggaaacccgcccaagcgaccccaacauaucagcaguugcccaaucccaacucccaacacaauccccaagcaacgccgcc

SEQ ID NO: 70 - RNA, 79 nt, Artificial Sequence
  source: /note=“Description of Artificial Sequence: Synthetic oligonucleotide”
ggaaagcgauugaaggcgucuuuucaacuacucgauuaagguuggguaucgucgugggacuuggaaauuuguuguuucc

SEQ ID NO: 71 - RNA, 79 nt, Artificial Sequence
  source: /note=“Description of Artificial Sequence: Synthetic oligonucleotide”
ggaaacuaaucgaaauaaaagagccccguacucuuuuauuucuauuagguuaggagccuuagcauuuguaucuuaggua

SEQ ID NO: 72 - RNA, 79 nt, Artificial Sequence
  source: /note=“Description of Artificial Sequence: Synthetic oligonucleotide”
ggaaaugugauuuccagcaacuucuuuugaauauauugaauuccuaauucaaagcgaacaaaucuacaagccauauacc

SEQ ID NO: 73 - RNA, 74 nt, Artificial Sequence
  source: /note=“Description of Artificial Sequence: Synthetic oligonucleotide”
ggaaaucguagagagucguacuuacguggucgccauugcauagcgcgcgaaagcaacaggaacaagaacgcgcc

SEQ ID NO: 74 - RNA, 74 nt, Artificial Sequence
  source: /note=“Description of Artificial Sequence: Synthetic oligonucleotide”
ggaaaucguagagagucguacuuagaauaaacagagucgggucgacuugucucugauacuacgacgucacaauc

SEQ ID NO: 75 - RNA, 79 nt, Artificial Sequence
  source: /note=“Description of Artificial Sequence: Synthetic oligonucleotide”
ggaaaauuugccuucggaguugcguauccugaacugcccagccuccugauauacaacuguuccgcuuauucgggccgcc

SEQ ID NO: 76 - RNA, 79 nt, Artificial Sequence
  source: /note=“Description of Artificial Sequence: Synthetic oligonucleotide”
ggaaaucugagcaggaauccuuugugcauugaagacuuuagauuccucucugcgguagacgugcacuuauaaguauuug

SEQ ID NO: 77 - RNA, 79 nt, Artificial Sequence
  source: /note=“Description of Artificial Sequence: Synthetic oligonucleotide”
ggaaagcgauugaaggcgucuuuucaacuacucgauuaagguuggguaucgucgugggacuuggaaauuuguugccacc

SEQ ID NO: 78 - RNA, 79 nt, Artificial Sequence
  source: /note=“Description of Artificial Sequence: Synthetic oligonucleotide”
ggaaaauuuuagccuggaacguuagauaacuguccuguugucuuuauauacuugguccccaaguaguuugucuuccaaa

SEQ ID NO: 79 - RNA, 110 nt, Artificial Sequence
  source: /note=“Description of Artificial Sequence: Synthetic polynucleotide”
gcuggagccucucacacaccucugccccuugggccucccacucccauggcucugggcgguccagaaggagcguacccccguggucuuugaauaaagucugagugggcggc

SEQ ID NO: 80 - RNA, 100 nt, Artificial Sequence
  source: /note=“Description of Artificial Sequence: Synthetic polynucleotide”
gcuggagccuccaccgcguuauccguuccucguaggcugguccuggggaacgggucggcggguacccccguggucuuugaauaaagucugagugggcggc

SEQ ID NO: 81 - RNA, 248 nt, Artificial Sequence
  source: /note=“Description of Artificial Sequence: Synthetic polynucleotide”
gcuggagccucugcccggcaacggccaggucugugccaaguguuugcugacgcaacccccacuggcuggggcuuggucaugggccaucagcgcgugcguggaaccuuuucggcuccucugccgauccauacugcggaacuccuagccgcuuguuuugcucgcagcaggucuggagcaaacauuaucgggacugauaacucuguuguccuguacccccguggucuuugaauaaagucugagugggcggc

SEQ ID NO: 82 - RNA, 210 nt, Artificial Sequence
  source: /note=“Description of Artificial Sequence: Synthetic polynucleotide”
gcuggagccucgguggccuagcuucuugccccuugggccuccccccagccccuccuccccuuccugcacccguacccuuuuuuuuuuuuuuuuuuucuucuuuucuuuuuuuucuuuuuuuuuuuucuuucuuuuuuucuuuuuuuuucuuuucuuuuuucuuuuuuuuuuuuuuuuccguggucuuugaauaaagucugagugggcggc

SEQ ID NO: 83 - RNA, 210 nt, Artificial Sequence
  source: /note=“Description of Artificial Sequence: Synthetic polynucleotide”
gcuggagccucgguggccuagcuucuugccccuugggccuccccccagccccuccuccccuuccugcacccguacccuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuccguggucuuugaauaaagucugagugggcggc

SEQ ID NO: 84 - RNA, 176 nt, Artificial Sequence
  source: /note=“Description of Artificial Sequence: Synthetic polynucleotide”
auaaaguaggaaacacuacagcuggagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 85 - RNA, 130 nt, Artificial Sequence
  source: /note=“Description of Artificial Sequence: Synthetic polynucleotide”
ggagccucgguggccuagcuucuugccccuugggccuccccccagccccuccuccccuuccugcacccguacccccuccauaaaguaggaaacacuacaguggucuuugaauaaagucugagugggcggc

SEQ ID NO: 86 - RNA, 179 nt, Artificial Sequence
  source: /note=“Description of Artificial Sequence: Synthetic polynucleotide”
uccauaaaguaggaaacacuacagcuggagccuccugagagaccugugugaacuauugagaagaucggaacagcuccuuacucugaggaaguuguccauaaaguaggaaacacuacaguacccccuccauaaaguaggaaacacuacaguggucuuugaauaaagucugagugggcggc

SEQ ID NO: 87 - RNA, 232 nt, Artificial Sequence
  source: /note=“Description of Artificial Sequence: Synthetic polynucleotide”
accucacucacggccacauugagugccaggcuccgggcugguuuauaguaguguagagcauugcagcacuuagacuggggugcuguagucuuuauuguagucuuuccacauaccugauaauucuuagauaauuucuuauuuuaauuccauaaaguaggaaacacuacauaaaucuccauaaaguaggaaacacuacauauucuuccauaaaguaggaaacacuacauaggcu

SEQ ID NO: 88 - RNA, 104 nt, Artificial Sequence
  source: /note=“Description of Artificial Sequence: Synthetic polynucleotide”
ggaaauuuuuuuuugauauuauaagaguuuuuuuuugauauuaagaaaauuuuuuuuugauauuagaagaguaagaagaaauauaagaccccggcgccgccacc

SEQ ID NO: 89 - RNA, 57 nt, Artificial Sequence
  source: /note=“Description of Artificial Sequence: Synthetic oligonucleotide”
ggaaauaagagagaaaagaagaguaagaagaaauauaagagccaaaaaaaaaaaacc

SEQ ID NO: 90 - RNA, 76 nt, Artificial Sequence
  source: /note=“Description of Artificial Sequence: Synthetic oligonucleotide”
ggaaaucucccugagcuucagggaguaagagagaaaagaagaguaagaagaaauauaagaccccggcgccgccacc

SEQ ID NO: 91 - RNA, 50 nt, Human betaherpesvirus 5
caccgcguuauccguuccucguaggcugguccuggggaacgggucggcgg

SEQ ID NO: 92 - RNA, 100 nt, Human betaherpesvirus 5
ccacccccagcgccaccaccgcugccgucgccaccgcguuauccguuccucguaggcugguccuggggaacgggucggcggccggucggcuucuguuuua

SEQ ID NO: 93 - RNA, 9 nt, Artificial Sequence
  source: /note=“Description of Artificial Sequence: Synthetic oligonucleotide”
ugauaguaa

SEQ ID NO: 94 - RNA, 94 nt, Artificial Sequence
  source: /note=“Description of Artificial Sequence: Synthetic oligonucleotide”
gccuccaccgcguuauccguuccucguaggcugguccuggggaacgggucggcggguacccccguggucuuugaauaaagucugagugggcggc

SEQ ID NO: 95 - RNA, 254 nt, Artificial Sequence
  source: /note=“Description of Artificial Sequence: Synthetic polynucleotide”
caccgcguuauccguuccucguaggcugguccuggggaacgggucggcgggccucgguggccuagcuucuugccccuugggcccaccgcguuauccguuccucguaggcugguccuggggaacgggucggcgguccccccagccccuccuccccuuccugcacccguaccccccaccgcguuauccguuccucguaggcugguccuggggaacgggucggcggguggucuuugaauaaagucugagugggcggc

SEQ ID NO: 96 - RNA, 9 nt, Artificial Sequence
  source: /note=“Description of Artificial Sequence: Synthetic oligonucleotide”
uaaagcgcu

SEQ ID NO: 97 - RNA, 119 nt, Artificial Sequence
  source: /note=“Description of Artificial Sequence: Synthetic polynucleotide”
uaauaguaagcuggagccuccugagagaccugugugaacuauugagaagaucggaacagcuccuuacucugaggaaguugguacccccguggucuuugaauaaagucugagugggcggc

SEQ ID NO: 98 - RNA, 119 nt, Artificial Sequence
  source: /note=“Description of Artificial Sequence: Synthetic polynucleotide”
uaauaguaagcuggagccucacucuccucuccaucccguauccaggcugugaauuuuucaaggaauauaaagaucgggauguacccccguggucuuugaauaaagucugagugggcggc

SEQ ID NO: 99 - RNA, 119 nt, Artificial Sequence
  source: /note=“Description of Artificial Sequence: Synthetic polynucleotide”
ugauaguaagcuggagccucuagugacggcaacagggcuugguuuuuccuuguugugaaaucgacaucucugaagacaggguacccccguggucuuugaauaaagucugagugggcggc

SEQ ID NO: 100 - RNA, 119 nt, Artificial Sequence
  source: /note=“Description of Artificial Sequence: Synthetic polynucleotide”
ugauaguaagcuggagccuccuuccaucuagucacaaagacuccuucguccccaguugccgucuaggauugggccucccaguacccccguggucuuugaauaaagucugagugggcggc

SEQ ID NO: 101 - RNA, 119 nt, Artificial Sequence
  source: /note=“Description of Artificial Sequence: Synthetic polynucleotide”
ugauaguaagcuggagccucccauaacaugacauaucuggauuuugugcuuagaaccuuaaauuggaagcauucuuaauuguacccccguggucuuugaauaaagucugagugggcggc

SEQ ID NO: 102 - RNA, 119 nt, Artificial Sequence
  source: /note=“Description of Artificial Sequence: Synthetic polynucleotide”
uaauaguaagcuggagccuccggaaaacuaaaauagagauauuucaagauuuuauaauuuucaaagaccuuugaaauauuguacccccguggucuuugaauaaagucugagugggcggc

SEQ ID NO: 103 - RNA, 119 nt, Artificial Sequence
  source: /note=“Description of Artificial Sequence: Synthetic polynucleotide”
uaauaguaagcuggagccucuacacauugcuucuaguuggcagaaauaauugauuaaaagaccagaaacugugauaacugguacccccguggucuuuaaauaaagucuaagugggcggc

SEQ ID NO: 104 - RNA, 119 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguacccccguggucuuugaauaaagucugagugggcggc

SEQ ID NO: 105 - RNA, 119 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguacccccguggucuuugaauaaagucugagugggcggc

SEQ ID NO: 106 - RNA, 141 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguaccccccaaacaccauugucacacuccaguggucuuugaauaaagucugagugggcggc

SEQ ID NO: 107 - RNA, 141 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguaccccccaaacaccauugucacacuccaguggucuuugaauaaagucugagugggcggc

SEQ ID NO: 108 - RNA, 142 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguacccccuccauaaaguaggaaacacuacaguggucuuugaauaaagucugagugggcggc

SEQ ID NO: 109 - RNA, 141 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguaccccccgcauuauuacucacgguacgaguggucuuugaauaaagucugagugggcggc

SEQ ID NO: 110 - RNA, 164 nt, Artificial Sequence
  source: /note=“Description of Artificial Sequence: Synthetic polynucleotide”
ugauaauaguccauaaaguaggaaacacuacagcuggagccucgguggccaugcuucuugccccuugggccuccccccagccccuccuccccuuccugcacccguaccccccgcauuauuacucacgguacgaguggucuuugaauaaagucugagugggcggc

SEQ ID NO: 111 - RNA, 188 nt, Artificial Sequence
  source: /note=“Description of Artificial Sequence: Synthetic polynucleotide”
ugauaauaguccauaaaguaggaaacacuacagcuggagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 112 - RNA, 119 nt, Artificial Sequence
  source: /note=“Description of Artificial Sequence: Synthetic polynucleotide”
ugauaauaggcuggagccucucacacaccucugccccuugggccucccacucccauggcucugggcgguccagaaggagcguacccccguggucuuugaauaaagucugagugggcggc

SEQ ID NO: 113 - RNA, 109 nt, Artificial Sequence
  source: /note=“Description of Artificial Sequence: Synthetic polynucleotide”
ugauaauaggcuggagccuccaccgcguuauccguuccucguaggcugguccuggggaacgggucggcggguacccccguggucuuugaauaaagucugagugggcggc

SEQ ID NO: 114 - RNA, 257 nt, Artificial Sequence
  source: /note=“Description of Artificial Sequence: Synthetic polynucleotide”
ugauaauaggcuggagccucugcccggcaacggccaggucugugccaaguguuugcugacgcaacccccacuggcuggggcuuggucaugggccaucagcgcgugcguggaaccuuuucggcuccucugccgauccauacugcggaacuccuagccgcuuguuuugcucgcagcaggucuggagcaaacauuaucgggacugauaacucuguuguccuguacccccguggucuuugaauaaagucugagugggcggc

SEQ ID NO: 115 - RNA, 219 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguacccuuuuuuuuuuuuuuuuuuucuucuuuucuuuuuuuucuuuuuuuuuuuucuuucuuuuuuucuuuuuuuuucuuuucuuuuuucuuuuuuuuuuuuuuuuccguggucuuugaauaaagucugagugggcggc

SEQ ID NO: 116 - RNA, 219 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguacccuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuccguggucuuugaauaaagucugagugggcggc

SEQ ID NO: 117 - RNA, 185 nt, Artificial Sequence
  source: /note=“Description of Artificial Sequence: Synthetic polynucleotide”
uaagucuccauaaaguaggaaacacuacagcuggagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 118 - RNA, 139 nt, Artificial Sequence
  source: /note=“Description of Artificial Sequence: Synthetic polynucleotide”
uaaagcgcuggagccucgguggccuagcuucuugccccuugggccuccccccagccccuccuccccuuccugcacccguacccccuccauaaaguaggaaacacuacaguggucuuugaauaaagucugagugggcggc

SEQ ID NO: 119 - RNA, 194 nt, Artificial Sequence
  source: /note=“Description of Artificial Sequence: Synthetic polynucleotide”
uaaagcuccccgggguccauaaaguaggaaacacuacagcuggagccuccugagagaccugugugaacuauugagaagaucggaacagcuccuuacucugaggaaguuguccauaaaguaggaaacacuacaguacccccuccauaaaguaggaaacacuacaguggucuuugaauaaagucugagugggcggc

SEQ ID NO: 120 - RNA, 241 nt, Artificial Sequence
  source: /note=“Description of Artificial Sequence: Synthetic polynucleotide”
uaauaguaaaccucacucacggccacauugagugccaggcuccgggcugguuuauaguaguguagagcauugcagcacuuagacuggggugcuguagucuuuauuguagucuuuccacauaccugauaauucuuagauaauuucuuauuuuaauuccauaaaguaggaaacacuacauaaaucuccauaaaguaggaaacacuacauauucuuccauaaaguaggaaacacuacauaggcu

SEQ ID NO: 121 - RNA, 100 nt, Artificial Sequence
  source: /note=“Description of Artificial Sequence: Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 122 - RNA, 119 nt, Artificial Sequence
  source: /note=“Description of Artificial Sequence: Synthetic polynucleotide”
uaagccccuccgggggccucgguggccuagcuucuugccccuugggccuccccccagccccuccuccccuuccugcacccguacccccguggucuuugaauaaagucugagugggcggc

SEQ ID NO: 123 - RNA, 150 nt, Artificial Sequence
  source: /note=“Description of Artificial Sequence: Synthetic polynucleotide”
  misc_feature (1)..(150): /note=“This sequence may encompass 80-150 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 124 - DNA, 126 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (126)..(126): inverted deoxythymidine
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuagaaaaaaaaaaaaaaaaaaaat

SEQ ID NO: 125 - RNA, 9 nt, Artificial Sequence
  source: /note=“Description of Artificial Sequence: Synthetic oligonucleotide”
ccrccaugg

SEQ ID NO: 126 - RNA, 109 nt, Artificial Sequence
  source: /note=“Description of Artificial Sequence: Synthetic polynucleotide”
uaaagcuccccgggggccuccaccgcguuauccguuccucguaggcugguccuggggaacgggucggcggguacccccguggucuuugaauaaagucugagugggcggc

SEQ ID NO: 127 - RNA, 269 nt, Artificial Sequence
  source: /note=“Description of Artificial Sequence: Synthetic polynucleotide”
uaaagcuccccggggcaccgcguuauccguuccucguaggcugguccuggggaacgggucggcgggccucgguggccuagcuucuugccccuugggcccaccgcguuauccguuccucguaggcugguccuggggaacgggucggcgguccccccagccccuccuccccuuccugcacccguaccccccaccgcguuauccguuccucguaggcugguccuggggaacgggucggcggguggucuuugaauaaagucugagugggcggc

SEQ ID NO: 128 - DNA, 21 bp, Artificial Sequence
  source: /note=“Description of Artificial Sequence: Synthetic oligonucleotide”
  modified_base (21)..(21): inverted deoxythymidine
aaaaaaaaaaaaaaaaaaaat

SEQ ID NO: 129 - RNA, 31 nt, Artificial Sequence
  source: /note=“Description of Artificial Sequence: Synthetic oligonucleotide”
cgagguggacuaagccccuccggggccaccg

SEQ ID NO: 130 - protein, 4 aa, Artificial Sequence
  source: /note=“Description of Artificial Sequence: Synthetic peptide”
KDEL